SEQUENCE LISTING (711 sequences)

SEQ ID NO: 1 - protein, 107 aa, Artificial Sequence
  Synthetic Construct
GQPREPQVYTLPPSRDELTKNQVSLSCAVKGFYPSDIAVEWESNGQPENCYKTTPPVLDSDGSFFLVSKLTVDKSRWQQGNVFSCSVMHEALHNHYTQKSLSLSPGK

SEQ ID NO: 2 - protein, 107 aa, Artificial Sequence
  Synthetic Construct
GQPREPQVYTLPPSRDELTKNQVSLWCLVKGFYPSDIAVEWESNGQPENNYKTTPPVLDCDGSFFLYSKLTVDKSRWQQGNVFSCSVMHEALHNHYTQKSLSLSPGK

SEQ ID NO: 3 - protein, 107 aa, Artificial Sequence
  Synthetic Construct
GQPREPQVYTLPPSRDELTKNQVSLSCAVKGFYPSDIAVEWESNGQPENNYKTTPPVLDCDGSFFLVSKLTVDKSRWQQGNVFSCSVMHEALHNHYTQKSLSLSPGK

SEQ ID NO: 4 - protein, 107 aa, Artificial Sequence
  Synthetic Construct
GQPREPQVYTLPPSRDELTKNQVSLWCLVKGFYPSDIAVEWESNGQPENCYKTTPPVLDSDGSFFLYSKLTVDKSRWQQGNVFSCSVMHEALHNHYTQKSLSLSPGK

SEQ ID NO: 5 - protein, 107 aa, Artificial Sequence
  Synthetic Construct
GQPREPQVYTLPPSRDELTKNQVSLTCVVKGFYPSDIAVEWESNGQPENCYKTTPPVLDSDGSFFLVSKLTVDKSRWQQGNVFSCSVMHEALHNHYTQKSLSLSPGK

SEQ ID NO: 6 - protein, 107 aa, Artificial Sequence; identical to SEQ ID NO: 2
  Synthetic Construct

SEQ ID NO: 7 - protein, 107 aa, Artificial Sequence
  Synthetic Construct
GQPREPQVYTLPPSRDELTKNQVSLTCVVKGFYPSDIAVEWESNGQPENNYKTTPPVLDCDGSFFLVSKLTVDKSRWQQGNVFSCSVMHEALHNHYTQKSLSLSPGK

SEQ ID NO: 8 - protein, 107 aa, Artificial Sequence; identical to SEQ ID NO: 4
  Synthetic Construct

SEQ ID NO: 9 - protein, 107 aa, Artificial Sequence
  Synthetic Construct
GQPREPQVYTLPPSRDKLTKNQVSLTCLVKGFYPSDIAVEWESNGQPENNYKTTPPVLDSDGSFFLYSKLKVDKSRWQQGNVFSCSVMHEALHNHYTQKSLSLSPGK

SEQ ID NO: 10 - protein, 107 aa, Artificial Sequence
  Synthetic Construct
GQPREPQVYTDPPSRDELTKNQVSLTCLVDGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 107 aa, Artificial Sequence
  Synthetic Construct
GQPREPQVYTLPPSRDKLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 107 aa, Artificial Sequence; identical to SEQ ID NO: 10
  Synthetic Construct

SEQ ID NO: 13 - protein, 107 aa, Artificial Sequence
  Synthetic Construct
GQPREPQVYTLPPSRKKLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 107 aa, Artificial Sequence
  Synthetic Construct
GQPREPQVYTDPPSRDELTKNQVSLTCLVDGFYPSDIAVEWESNGQPENNYKTTPPVLDSDGSFFLYSKLTVDKSRWQQGNVFSCSVMHEALHNHYTQDSLSLSPGK

SEQ ID NO: 15 - protein, 107 aa, Artificial Sequence
  Synthetic Construct
GQPREPQVYTLPPSRDKLTKNQVKLTCLVKGFYPSDIAVEWESNGQPENCYKTTPPVLDSDGSFFLYSKLTVDKSRWQQGNVFSCSVMHEALHNHYTQKSLSLSPGK

SEQ ID NO: 16 - protein, 107 aa, Artificial Sequence
  Synthetic Construct
GQPREPQVYTDPPSRDELTKNQVSLTCLVDGFYPSDIAVEWESNGQPENNYKTTPPVLDCDGSFFLYSKLTVDKSRWQQGNVFSCSVMHEALHNHYTQKSLSLSPGK

SEQ ID NO: 17 - protein, 107 aa, Artificial Sequence
  Synthetic Construct
GQPREPQVYTLPPSRDKLTKNQVKLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 18 - protein, 107 aa, Artificial Sequence
  Synthetic Construct
GQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQKSLSLSPGK

SEQ ID NO: 19 - protein, 107 aa, Artificial Sequence
  Synthetic Construct
GQPREPQVYTLPPSRKKLTKNQVKLTCLVKGFYPSDIAVEWESNGQPENCYKTTPPVLDSDGSFFLYSKLTVDKSRWQQGNVFSCSVMHEALHNHYTQKSLSLSPGK

SEQ ID NO: 20 - protein, 107 aa, Artificial Sequence
  Synthetic Construct
GQPREPQVYTDPPSRDELTKNQVSLTCLVDGFYPSDIAVEWESNGQPENNYKTTPPVLDCDGSFFLYSKLTVDKSRWQQGNVFSCSVMHEALHNHYTQDSLSLSPGK

SEQ ID NO: 21 - protein, 107 aa, Artificial Sequence
  Synthetic Construct
GQPREPQVYTLPPSRKKLTKNQVKLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 22 - protein, 107 aa, Artificial Sequence
  Synthetic Construct
GQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 23 - protein, 107 aa, Artificial Sequence
  Synthetic Construct
GQPREPQVYTLPPSRDELTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 24 - protein, 107 aa, Artificial Sequence
  Synthetic Construct
GQPREPQVYTLPPSRDELTKNQVSLTCLVKGFYPSDIAVEWESNGQPENCYKTTPPVLDSDGSFFLYSKLTVDKSRWQQGNVFSCSVMHEALHNHYTQKSLSLSPGK

SEQ ID NO: 25 - protein, 107 aa, Artificial Sequence
  Synthetic Construct
GQPREPQVYTLPPSRDELTKNQVSLTCLVKGFYPSDIAVEWESNGQPENNYCTTPPVLDSDGSFFLYSKLTVDKSRWQQGNVFSCSVMHEALHNHYTQKSLSLSPGK

SEQ ID NO: 26 - protein, 107 aa, Artificial Sequence
  Synthetic Construct
GQPREPQVYTLPPSRDELTKNQVSLTCLVKGFYPSDIAVEWESNGQPENNYKTTPPCLDSDGSFFLYSKLTVDKSRWQQGNVFSCSVMHEALHNHYTQKSLSLSPGK

SEQ ID NO: 27 - protein, 107 aa, Artificial Sequence; identical to SEQ ID NO: 25
  Synthetic Construct

SEQ ID NO: 28 - protein, 107 aa, Artificial Sequence; identical to SEQ ID NO: 23
  Synthetic Construct

SEQ ID NO: 29 - protein, 107 aa, Artificial Sequence
  Synthetic Construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 107 aa, Artificial Sequence
  Synthetic Construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 107 aa, Artificial Sequence
  Synthetic Construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 330 aa, Artificial Sequence [sequence omitted]

SEQ ID NO: 33 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 327 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 21 aa, Artificial Sequence
  Synthetic Construct
EVGSYPYDDPDCPSHDSDCDN

SEQ ID NO: 36 - protein, 24 aa, Artificial Sequence
  Synthetic Construct
ESDACDADPFDCQAGGGGSGSGGS

SEQ ID NO: 37 - protein, 10 aa, Artificial Sequence
  Synthetic Construct
YSISSGYYWG

SEQ ID NO: 38 - protein, 18 aa, Artificial Sequence
  Synthetic Construct
GIIYPSGGGTNYAQKFQG

SEQ ID NO: 39 - protein, 8 aa, Artificial Sequence
  Synthetic Construct
GGGLGFDY

SEQ ID NO: 40 - protein, 11 aa, Artificial Sequence
  Synthetic Construct
RASQSIPSFLN

SEQ ID NO: 41 - protein, 7 aa, Artificial Sequence
  Synthetic Construct
AASSLQS

SEQ ID NO: 42 - protein, 11 aa, Artificial Sequence
  Synthetic Construct
QHYISWPRQFT

SEQ ID NO: 43 - protein, 213 aa, Artificial Sequence
  Synthetic Construct
DIQLTQSPSSLSASVGDRVTITCRASSSVSYVAWYQQKPGKAPKRWIYDTSKLYSGVPSRFSGSGSGTDYTLTISSLQPEDFATYYCQQWSSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 449 aa, Artificial Sequence
  Synthetic Construct
EVQLVESGGGLVQPGGSLRLSCAASGYTFTRYTMHWVRQAPGKGLEWIGYINPSRGYTYYADSVKGRFTLSTDKSKNTAYLQMSSLRAEDTAVYYCARYYDDHYCLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGK

SEQ ID NO: 45 - protein, 475 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESDGSFALSSRLRVDKSRWQQGNVFSCSVMHEALHNHYTQKSLSLSPGK

SEQ ID NO: 46-58 - skipped (13 entries)

SEQ ID NO: 59 - protein, 116 aa, Artificial Sequence
  Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSS

SEQ ID NO: 60 - protein, 109 aa, Artificial Sequence
  Synthetic Construct
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKR

SEQ ID NO: 61 - protein, 9 aa, Artificial Sequence
  Synthetic Construct
FTFNTYAMN

SEQ ID NO: 62 - protein, 20 aa, Artificial Sequence
  Synthetic Construct
GRIRSKYNNYATYYADSVKG

SEQ ID NO: 63 - protein, 14 aa, Artificial Sequence
  Synthetic Construct
HGNFGNSYVSWFAY

SEQ ID NO: 64 - protein, 14 aa, Artificial Sequence
  Synthetic Construct
GSSTGAVTTSNYAN

SEQ ID NO: 65 - protein, 7 aa, Artificial Sequence
  Synthetic Construct
GTNKRAP

SEQ ID NO: 66 - protein, 7 aa, Artificial Sequence
  Synthetic Construct
WYSNLWV

SEQ ID NO: 67 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 68 - protein, 109 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 69 - protein, 9 aa, Artificial Sequence
  Synthetic Construct
FNIKDTYIH

SEQ ID NO: 70 - protein, 18 aa, Artificial Sequence
  Synthetic Construct
ARIYPTNGYTRYADSVKG

SEQ ID NO: 71 - protein, 11 aa, Artificial Sequence
  Synthetic Construct
WGGDGFYAMDY

SEQ ID NO: 72 - protein, 11 aa, Artificial Sequence
  Synthetic Construct
RASQDVNTAVA

SEQ ID NO: 73 - protein, 7 aa, Artificial Sequence
  Synthetic Construct
SASFLYS

SEQ ID NO: 74 - protein, 9 aa, Artificial Sequence
  Synthetic Construct
QQHYTTPPT

SEQ ID NO: 75 - protein, 120 aa, Artificial Sequence [sequence omitted]

SEQ ID NO: 76 - protein, 108 aa, Artificial Sequence
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 77 - protein, 13 aa, Artificial Sequence
  Synthetic Construct
GGGPLGLAGSGGS

SEQ ID NO: 78 - protein, 27 aa, Artificial Sequence
  Synthetic Construct
ESDACDADPFDCQAGGGPLGLAGSGGS

SEQ ID NO: 79 - protein, 255 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVLRGGGGSGGGGSGGGGSGGGGSEVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 80 - protein, 5 aa, Artificial Sequence
  Synthetic Construct
SGGGS

SEQ ID NO: 81 - protein, 9 aa, Artificial Sequence
  Synthetic Construct
GGGSGGGGS

SEQ ID NO: 82 - protein, 20 aa, Artificial Sequence
  Synthetic Construct
GGGGSGGGGSGGGGSGGGGS

SEQ ID NO: 83 - protein, 241 aa, Artificial Sequence
  Synthetic Construct
ESDACDADPFDCQAGGGPLGLAGSGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 450 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVKLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 85 - protein, 520 aa, Artificial Sequence
  Synthetic Construct
EVGSYPYDDPDCPSHESDCDQGGGPLGLAGSGGSQAVVTQEPSLTVSPGGTVTLTCGSSTGAVTTSNYPNWVQQKPGQAPRGLIGGTNKRAPGVPARFSGSLLGGKAALTLSGAQPEDEAEYYCALWYSNLWVFGGGTKLTVLGGGGSGGGGSGGGGSGGGGSEVQLVESGGGLVQPGGSLRLSCAASGFTFNTYAINWVRQAPGKGLEWVGRIRSKYNNYATYYAESVKGRFTISRDDSKNTLYLQINSLRAEDTAVYYCVRHGNFGTSYVSWFAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 86 - protein, 5 aa, Artificial Sequence
  Synthetic Construct
SYNIH

SEQ ID NO: 87 - protein, 121 aa, Artificial Sequence
  Synthetic Construct
EVQLVQSGAEVKKPGSSVKVSCKASGYTFTSYNIHWVRQAPGKGLEWIGAIYPGSGDTSYNQKFKGRVTITADKSTSTAYMELSSLRSEDTAVYYCARSTYYGGDWYFNVWGQGTLVTVSS

SEQ ID NO: 88 - protein, 121 aa, Artificial Sequence
  Synthetic Construct
EVQLVQSGAEVKKPGSSVKVSCKASGYTFTSYNMHWVRQAPGQGLEWIGAIYPGSGDTSYNQKFKGRVTITADKSISTAYMELSSLRSEDTAVYYCARSTYYGGDWYFNVWGQGTLVTVSS

SEQ ID NO: 89 - protein, 107 aa, Artificial Sequence
  Synthetic Construct
DIVLTQSPSSLSASVGDRVTITCRASSSVSYIHWYQQKPGKSPKPLIYATSNLASGVPVRFSGSGSGTDYTLTISSLQPEDFATYYCQQWTSNPPTFGQGTKVEIKR

SEQ ID NO: 90 - protein, 107 aa, Artificial Sequence
  Synthetic Construct
DIQLTQSPSSLSASVGDRVTITCRASSSVSYIHWFQQKPGKAPKLLIYATSNLASGVPSRFSGSGSGTDFTLTISSLQPEDFATYYCQQWTSNPPTFGQGTKVEIKR

SEQ ID NO: 91 - protein, 107 aa, Artificial Sequence
  Synthetic Construct
DIQLTQSPSSLSASVGDRVTITCRASSSVSYIHWYQQKPGKAPKPLIYATSNLASGVPSRFSGSGSGTDFTLTISSLQPEDFATYYCQQWTSNPPTFGQGTKVEIKR

SEQ ID NO: 92 - protein, 107 aa, Artificial Sequence
  Synthetic Construct
QIVLSQSPAILSASPGEKVTMTCRASSSVSYIHWFQQKPGKSPKPWIYATSNLASGVPVRFSGSGSGTSYSLTISRVEAEDAATYYCQQWTSNPPTFGGGTKVEIKR

SEQ ID NO: 93-111 - skipped (19 entries)

SEQ ID NO: 112 - protein, 214 aa, Artificial Sequence
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 450 aa, Artificial Sequence; identical to SEQ ID NO: 84
  Synthetic Construct

SEQ ID NO: 114 - protein, 487 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVLRGGGGSGGGGSGGGGSGGGGSEVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 115 - protein, 238 aa, Artificial Sequence
  Synthetic Construct
ESDACDADPFDCQAPLGLAGSGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 450 aa, Artificial Sequence; identical to SEQ ID NO: 84
  Synthetic Construct

SEQ ID NO: 117 - protein, 528 aa, Artificial Sequence
  Synthetic Construct
EVGSYPYDDPDCPSHDSDCDNSGRSAGGGGTPLGLAGSGGSQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVLRGGGGSGGGGSGGGGSGGGGSEVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 118-126 - skipped (9 entries)

SEQ ID NO: 127 - protein, 5 aa, Artificial Sequence
  Synthetic Construct
SGRSA

SEQ ID NO: 128 - protein, 6 aa, Artificial Sequence
  Synthetic Construct
PLGLAG

SEQ ID NO: 129 - protein, 7 aa, Artificial Sequence
  Synthetic Construct
ENLYFQG

SEQ ID NO: 130 - protein, 5 aa, Artificial Sequence
  Synthetic Construct
GGGGS

SEQ ID NO: 131 - protein, 4 aa, Artificial Sequence
  Synthetic Construct
SGGS

SEQ ID NO: 132 - protein, 4 aa, Artificial Sequence
  Synthetic Construct
GGSG

SEQ ID NO: 133 - protein, 5 aa, Artificial Sequence
  Synthetic Construct
GGSGG

SEQ ID NO: 134 - protein, 5 aa, Artificial Sequence
  Synthetic Construct
GSGSG

SEQ ID NO: 135 - protein, 5 aa, Artificial Sequence
  Synthetic Construct
GSGGG

SEQ ID NO: 136 - protein, 5 aa, Artificial Sequence
  Synthetic Construct
GGGSG

SEQ ID NO: 137 - protein, 5 aa, Artificial Sequence
  Synthetic Construct
GSSSG

SEQ ID NO: 138-365 - skipped (228 entries)

SEQ ID NO: 366 - protein, 118 aa, Artificial Sequence
  Synthetic Construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYLDYWGQGTPVTVSS

SEQ ID NO: 367 - protein, 106 aa, Artificial Sequence [sequence omitted]

SEQ ID NO: 368 - protein, 5 aa, Artificial Sequence
  Synthetic Construct
RYTMH

SEQ ID NO: 369 - protein, 17 aa, Artificial Sequence
  Synthetic Construct
YINPSRGYTNYNQKVKD

SEQ ID NO: 370 - protein, 9 aa, Artificial Sequence
  Synthetic Construct
YYDDHYLDY

SEQ ID NO: 371 - protein, 10 aa, Artificial Sequence
  Synthetic Construct
SASSSVSYMN

SEQ ID NO: 372 - protein, 7 aa, Artificial Sequence
  Synthetic Construct
DTSKLAS

SEQ ID NO: 373 - protein, 9 aa, Artificial Sequence
  Synthetic Construct
QQWSSNPFT

SEQ ID NO: 374 - protein, 125 aa, Artificial Sequence
  Synthetic Construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 375 - protein, 109 aa, Artificial Sequence
  Synthetic Construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 376 - protein, 5 aa, Artificial Sequence
  Synthetic Construct
TYAMN

SEQ ID NO: 377 - protein, 19 aa, Artificial Sequence
  Synthetic Construct
RIRSKYNNYATYYADSVKD

SEQ ID NO: 378 - protein, 14 aa, Artificial Sequence
  Synthetic Construct
HGNFGNSYVSWFAY

SEQ ID NO: 379 - protein, 14 aa, Artificial Sequence
  Synthetic Construct
RSSTGAVTTSNYAN

SEQ ID NO: 380 - protein, 7 aa, Artificial Sequence
  Synthetic Construct
GTNKRAP

SEQ ID NO: 381 - protein, 9 aa, Artificial Sequence
  Synthetic Construct
ALWYSNLWV

SEQ ID NO: 382 - protein, 5 aa, Artificial Sequence
  Synthetic Construct
  VARIANT 1, 4, 5: Xaa = D or S or T
  VARIANT 4: Xaa = I or L or M
  VARIANT 5: Xaa = N or T
XYAXX

SEQ ID NO: 383 - protein, 19 aa, Artificial Sequence
  Synthetic Construct
  VARIANT 15: Xaa =  D or E
  VARIANT 16: Xaa = S or T
  VARIANT 19: Xaa = D or G or S
RIRSKYNNYATYYAXXVKX

SEQ ID NO: 384 - protein, 14 aa, Artificial Sequence
  Synthetic Construct
  VARIANT 4: Xaa = F or Y
  VARIANT 6: Xaa = N or T
  VARIANT 11: Xaa = W or Y
  VARIANT 12: Xaa = F or W
HGNXGXSYVSXXAY

SEQ ID NO: 385 - protein, 14 aa, Artificial Sequence
  Synthetic Construct
  VARIANT 1: Xaa = A or G or R
  VARIANT 9: Xaa = S or T
  VARIANT 10: Xaa = G or S
  VARIANT 13: Xaa = A or P or V
XSSTGAVTXXNYXN

SEQ ID NO: 386 - protein, 7 aa, Artificial Sequence
  Synthetic Construct
  VARIANT 3: Xaa = K or N
  VARIANT 4: Xaa = F or K
GTXXRAP

SEQ ID NO: 387 - protein, 9 aa, Artificial Sequence
  Synthetic Construct
  VARIANT 6: Xaa = D or N or T
  VARIANT 7: Xaa = L or R
ALWYSXXWV

SEQ ID NO: 388 - protein, 125 aa, Artificial Sequence
  Synthetic Construct
  VARIANT 13: Xaa = K or Q
  VARIANT 30: Xaa = N or S
  VARIANT 31: Xaa = S or T
  VARIANT 35: Xaa = H or N
  VARIANT 49: Xaa = G or S
  VARIANT 64: Xaa = D or E
  VARIANT 68: Xaa = D or G
  VARIANT 76: Xaa = D or N
  VARIANT 85: Xaa = I or L
  VARIANT 99: Xaa = A or V
  VARIANT 104: Xaa = F or Y
  VARIANT 106: Xaa = N or T
EVQLVESGGGLVXPGGSLRLSCAASGFTFXXYAIXWVRQAPGKGLEWVXRIRSKYNNYATYYAXSVKXRFTISRDXSKNTLYLQXNSLRAEDTAVYYCXRHGNXGXSYVSWFAYWGQGTLVTVSS

SEQ ID NO: 389 - protein, 109 aa, Artificial Sequence
  Synthetic Construct
  VARIANT 1, 23, 35, 38, 54, 55, 60, 96, 97: Xaa = E or Q
  VARIANT 23: Xaa = A or G or P or R
  VARIANT 35: Xaa = A or P
  VARIANT 38: Xaa = F or V
  VARIANT 54: Xaa = K or N
  VARIANT 55: Xaa = F or K
  VARIANT 60: Xaa = A or I or T or V
  VARIANT 96: Xaa = A or D or N or T
  VARIANT 97: Xaa = H or L
XAVVTQEPSLTVSPGGTVTLTCXSSTGAVTTSNYXNWXQQKPGQAPRGLIGGTXXRAPGXPARFSGSLLGGKAALTLSGAQPEDEAEYYCALWYSXXWVFGGGTKLTVL

SEQ ID NO: 390 - protein, 5 aa, Artificial Sequence
  Synthetic Construct
TYAIN

SEQ ID NO: 391 - protein, 19 aa, Artificial Sequence
  Synthetic Construct
RIRSKYNNYATYYADSVKG

SEQ ID NO: 392 - protein, 19 aa, Artificial Sequence
  Synthetic Construct
RIRSKYNNYATYYAESVKD

SEQ ID NO: 393 - protein, 19 aa, Artificial Sequence
  Synthetic Construct
RIRSKYNNYATYYADSVKD

SEQ ID NO: 394 - protein, 19 aa, Artificial Sequence
  Synthetic Construct
RIRSKYNNYATYYAESVKG

SEQ ID NO: 395 - protein, 14 aa, Artificial Sequence
  Synthetic Construct
HGNFGTSYVSWFAY

SEQ ID NO: 396 - protein, 14 aa, Artificial Sequence
  Synthetic Construct
GSSTGAVTTSNYAN

SEQ ID NO: 397 - protein, 14 aa, Artificial Sequence
  Synthetic Construct
GSSTGAVTTSNYPN

SEQ ID NO: 398 - protein, 14 aa, Artificial Sequence
  Synthetic Construct
RSSTGAVTTSNYAN

SEQ ID NO: 399 - protein, 7 aa, Artificial Sequence
  Synthetic Construct
GTKFRAP

SEQ ID NO: 400 - protein, 9 aa, Artificial Sequence
  Synthetic Construct
ALWYSDLWV

SEQ ID NO: 401 - protein, 9 aa, Artificial Sequence
  Synthetic Construct
ALWYSTLWV

SEQ ID NO: 402 - protein, 125 aa, Artificial Sequence
  Synthetic Construct
EVQLVESGGGLVQPGGSLRLSCAASGFTFSTYAINWVRQAPGKGLEWVSRIRSKYNNYATYYAESVKDRFTISRDDSKNTLYLQINSLRAEDTAVYYCVRHGNFGTSYVSWFAYWGQGTLVTVSS

SEQ ID NO: 403 - protein, 109 aa, Artificial Sequence
  Synthetic Construct
EAVVTQEPSLTVSPGGTVTLTCGSSTGAVTTSNYPNWVQQKPGQAPRGLIGGTNKRAPGTPARFSGSLLGGKAALTLSGAQPEDEAEYYCALWYSDLWVFGGGTKLTVL

SEQ ID NO: 404 - protein, 109 aa, Artificial Sequence
  Synthetic Construct
QAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSTLWVFGGGTKLTVL

SEQ ID NO: 405 - protein, 125 aa, Artificial Sequence
  Synthetic Construct
EVQLVESGGGLVQPGGSLRLSCAASGFTFNTYAINWVRQAPGKGLEWVSRIRSKYNNYATYYADSVKDRFTISRDDSKNTLYLQINSLRAEDTAVYYCVRHGNFGTSYVSWFAYWGQGTLVTVSS

SEQ ID NO: 406 - protein, 109 aa, Artificial Sequence
  Synthetic Construct
QAVVTQEPSLTVSPGGTVTLTCGSSTGAVTTSNYPNWVQQKPGQAPRGLIGGTNKRAPGTPARFSGSLLGGKAALTLSGAQPEDEAEYYCALWYSNLWVFGGGTKLTVL

SEQ ID NO: 407 - protein, 125 aa, Artificial Sequence
  Synthetic Construct
EVQLVESGGGLVKPGGSLRLSCAASGFTFSTYAMNWVRQAPGKGLEWVSRIRSKYNNYATYYADSVKDRFTISRDDSKNTLYLQINSLRAEDTAVYYCVRHGNFGTSYVSWFAYWGQGTLVTVSS

SEQ ID NO: 408 - protein, 109 aa, Artificial Sequence
  Synthetic Construct
EAVVTQEPSLTVSPGGTVTLTCRSSTGAVTTSNYANWVQQKPGQAPRGLIGGTKFRAPGVPARFSGSLLGGKAALTLSGAQPEDEAEYYCALWYSDLWVFGGGTKLTVL

SEQ ID NO: 409 - protein, 125 aa, Artificial Sequence
  Synthetic Construct
EVQLVESGGGLVQPGGSLRLSCAASGFTFSTYAMNWVRQAPGKGLEWVSRIRSKYNNYATYYADSVKDRFTISRDDSKNTLYLQINSLRAEDTAVYYCVRHGNFGTSYVSWFAYWGQGTLVTVSS

SEQ ID NO: 410 - protein, 125 aa, Artificial Sequence
  Synthetic Construct
EVQLVESGGGLVQPGGSLRLSCAASGFTFNTYAINWVRQAPGKGLEWVGRIRSKYNNYATYYAESVKGRFTISRDDSKNTLYLQINSLRAEDTAVYYCVRHGNFGTSYVSWFAYWGQGTLVTVSS

SEQ ID NO: 411 - protein, 109 aa, Artificial Sequence
  Synthetic Construct
QAVVTQEPSLTVSPGGTVTLTCGSSTGAVTTSNYPNWVQQKPGQAPRGLIGGTNKRAPGVPARFSGSLLGGKAALTLSGAQPEDEAEYYCALWYSNLWVFGGGTKLTVL

SEQ ID NO: 412 - protein, 125 aa, Artificial Sequence
  Synthetic Construct
EVQLVESGGGLVQPGGSLRLSCAASGFTFNTYAINWVRQAPGKGLEWVGRIRSKYNNYATYYADSVKGRFTISRDDSKNTLYLQINSLRAEDTAVYYCVRHGNFGTSYVSWFAYWGQGTLVTVSS

SEQ ID NO: 413 - protein, 109 aa, Artificial Sequence
  Synthetic Construct
QAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 414 - protein, 125 aa, Artificial Sequence
  Synthetic Construct
EVQLVESGGGLVQPGGSLRLSCAASGFTFNTYAMNWVRQAPGKGLEWVSRIRSKYNNYATYYADSVKGRFTISRDDSKNTLYLQLNSLRAEDTAVYYCVRHGNFGNSYVSWFAYWGQGTLVTVSS

SEQ ID NO: 415 - protein, 125 aa, Artificial Sequence
  Synthetic Construct
EVQLVESGGGLVQPGGSLRLSCAASGFTFNTYAINWVRQAPGKGLEWVGRIRSKYNNYATYYADSVKDRFTISRDDSKNTLYLQINSLRAEDTAVYYCVRHGNFGNSYVSWFAYWGQGTLVTVSS

SEQ ID NO: 416 - protein, 125 aa, Artificial Sequence
  Synthetic Construct
EVQLVESGGGLVQPGGSLRLSCAASGFTFNTYAINWVRQAPGKGLEWVGRIRSKYNNYATYYADSVKGRFTISRDDSKNTLYLQINSLRAEDTAVYYCVRHGNFGNSYVSWFAYWGQGTLVTVSS

SEQ ID NO: 417 - protein, 21 aa, Artificial Sequence
  Synthetic Construct
EVGSYPYDDPDCPSHESDCDQ

SEQ ID NO: 418 - protein, 11 aa, Artificial Sequence
  Synthetic Construct
GGGPLGLAGGS

SEQ ID NO: 419 - protein, 14 aa, Artificial Sequence
  Synthetic Construct
ESDACDADPFDCQA

SEQ ID NO: 420 - protein, 10 aa, Artificial Sequence
  Synthetic Construct
PLGLAGSGGS

SEQ ID NO: 421 - protein, 254 aa, Artificial Sequence
  Synthetic Construct
EAVVTQEPSLTVSPGGTVTLTCGSSTGAVTTSNYPNWVQQKPGQAPRGLIGGTNKRAPGTPARFSGSLLGGKAALTLSGAQPEDEAEYYCALWYSDLWVFGGGTKLTVLGGGGSGGGGSGGGGSGGGGSEVQLVESGGGLVQPGGSLRLSCAASGFTFSTYAINWVRQAPGKGLEWVSRIRSKYNNYATYYAESVKDRFTISRDDSKNTLYLQINSLRAEDTAVYYCVRHGNFGTSYVSWFAYWGQGTLVTVSS

SEQ ID NO: 422 - protein, 254 aa, Artificial Sequence
  Synthetic Construct
QAVVTQEPSLTVSPGGTVTLTCGSSTGAVTTSNYPNWVQQKPGQAPRGLIGGTNKRAPGVPARFSGSLLGGKAALTLSGAQPEDEAEYYCALWYSNLWVFGGGTKLTVLGGGGSGGGGSGGGGSGGGGSEVQLVESGGGLVQPGGSLRLSCAASGFTFNTYAINWVRQAPGKGLEWVGRIRSKYNNYATYYAESVKGRFTISRDDSKNTLYLQINSLRAEDTAVYYCVRHGNFGTSYVSWFAYWGQGTLVTVSS

SEQ ID NO: 423 - protein, 5 aa, Artificial Sequence
  Synthetic Construct
DTYIH

SEQ ID NO: 424 - protein, 17 aa, Artificial Sequence
  Synthetic Construct
RIYPTNGYTRYADSVKG

SEQ ID NO: 425 - protein, 450 aa, Artificial Sequence; identical to SEQ ID NO: 84
  Synthetic Construct

SEQ ID NO: 426 - protein, 486 aa, Artificial Sequence
  Synthetic Construct
EAVVTQEPSLTVSPGGTVTLTCGSSTGAVTTSNYPNWVQQKPGQAPRGLIGGTNKRAPGTPARFSGSLLGGKAALTLSGAQPEDEAEYYCALWYSDLWVFGGGTKLTVLGGGGSGGGGSGGGGSGGGGSEVQLVESGGGLVQPGGSLRLSCAASGFTFSTYAINWVRQAPGKGLEWVSRIRSKYNNYATYYAESVKDRFTISRDDSKNTLYLQINSLRAEDTAVYYCVRHGNFGTSYVSWFAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 427 - protein, 450 aa, Artificial Sequence; identical to SEQ ID NO: 84
  Synthetic Construct

SEQ ID NO: 428 - protein, 486 aa, Artificial Sequence
  Synthetic Construct
QAVVTQEPSLTVSPGGTVTLTCGSSTGAVTTSNYPNWVQQKPGQAPRGLIGGTNKRAPGVPARFSGSLLGGKAALTLSGAQPEDEAEYYCALWYSNLWVFGGGTKLTVLGGGGSGGGGSGGGGSGGGGSEVQLVESGGGLVQPGGSLRLSCAASGFTFNTYAINWVRQAPGKGLEWVGRIRSKYNNYATYYAESVKGRFTISRDDSKNTLYLQINSLRAEDTAVYYCVRHGNFGTSYVSWFAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 429 - protein, 450 aa, Artificial Sequence; identical to SEQ ID NO: 84
  Synthetic Construct

SEQ ID NO: 430 - protein, 518 aa, Artificial Sequence
  Synthetic Construct
EVGSYPYDDPDCPSHESDCDQGGGPLGLAGGSEAVVTQEPSLTVSPGGTVTLTCGSSTGAVTTSNYPNWVQQKPGQAPRGLIGGTNKRAPGTPARFSGSLLGGKAALTLSGAQPEDEAEYYCALWYSDLWVFGGGTKLTVLGGGGSGGGGSGGGGSGGGGSEVQLVESGGGLVQPGGSLRLSCAASGFTFSTYAINWVRQAPGKGLEWVSRIRSKYNNYATYYAESVKDRFTISRDDSKNTLYLQINSLRAEDTAVYYCVRHGNFGTSYVSWFAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 431 - protein, 20 aa, Artificial Sequence
  Synthetic Construct
SGRSAGGGGTPLGLAGSGGS

SEQ ID NO: 432 - protein, 21 aa, Artificial Sequence
  Synthetic Construct
EVGSYHYASDACDADPFDCNA

SEQ ID NO: 433 - protein, 21 aa, Artificial Sequence
  Synthetic Construct
EVGSYAYIPPDCHADPYDCSV

SEQ ID NO: 434 - protein, 21 aa, Artificial Sequence
  Synthetic Construct
EVGSYANTNDPCTLDPYDCSH

SEQ ID NO: 435 - protein, 21 aa, Artificial Sequence
  Synthetic Construct
EVGSYTASNADCPYDPYTCYA

SEQ ID NO: 436 - protein, 21 aa, Artificial Sequence
  Synthetic Construct
EVGSYADALYDCYDADPDCYY

SEQ ID NO: 437 - protein, 21 aa, Artificial Sequence
  Synthetic Construct
EVGSYSFSDFACDTTPFDCFA

SEQ ID NO: 438 - protein, 21 aa, Artificial Sequence
  Synthetic Construct
EVGSYITPDCLFFDPFDCVDN

SEQ ID NO: 439 - protein, 21 aa, Artificial Sequence
  Synthetic Construct
EVGSYAADYCDPFDFSFCLST

SEQ ID NO: 440 - protein, 21 aa, Artificial Sequence
  Synthetic Construct
EVGSYPIAICAPHSSDDCAFT

SEQ ID NO: 441 - protein, 21 aa, Artificial Sequence
  Synthetic Construct
EVGSYHLALCLDPDDSSCNFA

SEQ ID NO: 442 - protein, 21 aa, Artificial Sequence
  Synthetic Construct
EVGSYSSTACIFIDPFDCSVA

SEQ ID NO: 443 - protein, 21 aa, Artificial Sequence
  Synthetic Construct
EVGSYDHDNYDCYDYYDNCYY

SEQ ID NO: 444 - protein, 21 aa, Artificial Sequence
  Synthetic Construct
EVGSYPFLVCDDASPFDCTLV

SEQ ID NO: 445 - protein, 21 aa, Artificial Sequence
  Synthetic Construct
EVGSYIAFYCPDAHPYDCTSL

SEQ ID NO: 446 - protein, 21 aa, Artificial Sequence
  Synthetic Construct
EVGSYPALDCATFPSAVCTAD

SEQ ID NO: 447 - protein, 23 aa, Artificial Sequence
  Synthetic Construct
EVGSYAPADFDCSVFADPFDCSS

SEQ ID NO: 448 - protein, 23 aa, Artificial Sequence
  Synthetic Construct
EVGSYNDYDHYCSAFPSDLACAN

SEQ ID NO: 449 - protein, 23 aa, Artificial Sequence
  Synthetic Construct
EVGSYDPTTDVCSYYSDPFDCYY

SEQ ID NO: 450 - protein, 23 aa, Artificial Sequence
  Synthetic Construct
EVGSYDSNDDYCAHDSDPYDCYY

SEQ ID NO: 451 - protein, 23 aa, Artificial Sequence
  Synthetic Construct
EVGSYPAPAYNCHATSDPYDCAN

SEQ ID NO: 452 - protein, 23 aa, Artificial Sequence
  Synthetic Construct
EVGSYDNDNYDCYDHHNDHDCYY

SEQ ID NO: 453 - protein, 23 aa, Artificial Sequence
  Synthetic Construct
EVGSYYDDDDDCYDNYYDHDCYY

SEQ ID NO: 454 - protein, 23 aa, Artificial Sequence
  Synthetic Construct
EVGSYFDLHDYCNTYHDFPDCAP

SEQ ID NO: 455 - protein, 23 aa, Artificial Sequence
  Synthetic Construct
EVGSYYADYTACAFANSPYDCDY

SEQ ID NO: 456 - protein, 23 aa, Artificial Sequence
  Synthetic Construct
EVGSYHADHANCVSSFDPYDCDN

SEQ ID NO: 457 - protein, 23 aa, Artificial Sequence
  Synthetic Construct
EVGSYNHPIDDCYNINDPYDCDH

SEQ ID NO: 458 - protein, 23 aa, Artificial Sequence
  Synthetic Construct
EVGSYPHDYHNCYDYYHYDHCHH

SEQ ID NO: 459 - protein, 23 aa, Artificial Sequence
  Synthetic Construct
EVGSYAHDYADCLHNSDPYDCPD

SEQ ID NO: 460 - protein, 23 aa, Artificial Sequence
  Synthetic Construct
EVGSYYNSDDDCASHYDPYTCYY

SEQ ID NO: 461 - protein, 23 aa, Artificial Sequence
  Synthetic Construct
EVGSYYDYSHDCHDLDHNHACDP

SEQ ID NO: 462 - protein, 23 aa, Artificial Sequence
  Synthetic Construct
EVGSYTHASDFCHTSIDPYDCAY

SEQ ID NO: 463 - protein, 21 aa, Artificial Sequence
  Synthetic Construct
EVGSYAALPLCFPLSFCFVPT

SEQ ID NO: 464 - protein, 21 aa, Artificial Sequence
  Synthetic Construct
EVGSYAPPNLCAVVLSCVVAP

SEQ ID NO: 465 - protein, 21 aa, Artificial Sequence
  Synthetic Construct
EVGSYTAIPFCPPAIFCDPFS

SEQ ID NO: 466 - protein, 23 aa, Artificial Sequence
  Synthetic Construct
EVGSYYDDYHDCDYYDDYDNCYD

SEQ ID NO: 467 - protein, 21 aa, Artificial Sequence
  Synthetic Construct
EVGSYSAPHSCSPFSACTPAD

SEQ ID NO: 468 - protein, 21 aa, Artificial Sequence
  Synthetic Construct
EVGSYYPDHDDCDDYHDHCDY

SEQ ID NO: 469 - protein, 21 aa, Artificial Sequence
  Synthetic Construct
EVGSYHYASDACDADPFDCQA

SEQ ID NO: 470 - protein, 21 aa, Artificial Sequence
  Synthetic Construct
EVGSYQTTSDTCSDADDTCSV

SEQ ID NO: 471 - protein, 23 aa, Artificial Sequence
  Synthetic Construct
EVGSYHYDDYDCDDDHDDYYCQY

SEQ ID NO: 472 - protein, 23 aa, Artificial Sequence
  Synthetic Construct
EVGSYHYDNYQCTNYDYDYDCAD

SEQ ID NO: 473 - protein, 21 aa, Artificial Sequence
  Synthetic Construct
EVGSYSANNLACHADPFDCTA

SEQ ID NO: 474 - protein, 16 aa, Artificial Sequence
  Synthetic Construct
EVGNLACHADPFDCTA

SEQ ID NO: 475 - protein, 14 aa, Artificial Sequence
  Synthetic Construct
ENLACHADPFDCTA

SEQ ID NO: 476 - protein, 16 aa, Artificial Sequence
  Synthetic Construct
EVGSDACDADPFDCQA

SEQ ID NO: 477 - protein, 1 aa, Artificial Sequence
  Synthetic Construct
G

SEQ ID NO: 478 - protein, 1 aa, Artificial Sequence
  Synthetic Construct
P

SEQ ID NO: 479 - protein, 2 aa, Artificial Sequence
  Synthetic Construct
PL

SEQ ID NO: 480 - protein, 3 aa, Artificial Sequence
  Synthetic Construct
PLG

SEQ ID NO: 481 - protein, 4 aa, Artificial Sequence
  Synthetic Construct
PLGL

SEQ ID NO: 482 - protein, 5 aa, Artificial Sequence
  Synthetic Construct
PLGLA

SEQ ID NO: 483 - protein, 8 aa, Artificial Sequence
  Synthetic Construct
PLGLAGGS

SEQ ID NO: 484 - protein, 11 aa, Artificial Sequence
  Synthetic Construct
GPLGLAGSGGS

SEQ ID NO: 485 - protein, 12 aa, Artificial Sequence
  Synthetic Construct
GGPLGLAGSGGS

SEQ ID NO: 486 - protein, 15 aa, Artificial Sequence
  Synthetic Construct
GGGGSPLGLAGSGGS

SEQ ID NO: 487 - protein, 20 aa, Artificial Sequence
  Synthetic Construct
GGGGSGGGGSPLGLAGSGGS

SEQ ID NO: 488 - protein, 25 aa, Artificial Sequence
  Synthetic Construct
GGGGSGGGGSGGGGSPLGLAGSGGS

SEQ ID NO: 489 - protein, 30 aa, Artificial Sequence
  Synthetic Construct
GGGGSGGGGSGGGGSPLGLAGGGGSGSGGS

SEQ ID NO: 490 - protein, 35 aa, Artificial Sequence
  Synthetic Construct
GGGGSGGGGSGGGGSPLGLAGGGGSGGGGSGSGGS

SEQ ID NO: 491 - protein, 14 aa, Artificial Sequence
  Synthetic Construct
ESEVCDADPFECQA

SEQ ID NO: 492 - protein, 14 aa, Artificial Sequence
  Synthetic Construct
ESEFCDADPFECQA

SEQ ID NO: 493 - protein, 14 aa, Artificial Sequence
  Synthetic Construct
ESEYCDADPFECQA

SEQ ID NO: 494 - protein, 14 aa, Artificial Sequence
  Synthetic Construct
ESEACDLDPFECQA

SEQ ID NO: 495 - protein, 14 aa, Artificial Sequence
  Synthetic Construct
ESEACDADPFECQF

SEQ ID NO: 496 - protein, 14 aa, Artificial Sequence
  Synthetic Construct
ESEACDADPFECQY

SEQ ID NO: 497 - protein, 14 aa, Artificial Sequence
  Synthetic Construct
ESEVCDLDPFECQF

SEQ ID NO: 498 - protein, 14 aa, Artificial Sequence
  Synthetic Construct
ESDACEADPFDCQA

SEQ ID NO: 499 - protein, 14 aa, Artificial Sequence
  Synthetic Construct
ESDVCDADPFDCQA

SEQ ID NO: 500 - protein, 14 aa, Artificial Sequence
  Synthetic Construct
ESDVCDLDPFDCQA

SEQ ID NO: 501 - protein, 14 aa, Artificial Sequence
  Synthetic Construct
ESDVCDLDPFDCQF

SEQ ID NO: 502 - protein, 14 aa, Artificial Sequence
  Synthetic Construct
ESEVCEADPFECQA

SEQ ID NO: 503 - protein, 21 aa, Artificial Sequence
  Synthetic Construct
EVGSYASAPDACDADPYECQA

SEQ ID NO: 504 - protein, 21 aa, Artificial Sequence
  Synthetic Construct
EVGSYASVTEACDADPYECSH

SEQ ID NO: 505 - protein, 21 aa, Artificial Sequence
  Synthetic Construct
EVGSYSDVPEVCEADPYECQH

SEQ ID NO: 506 - protein, 21 aa, Artificial Sequence
  Synthetic Construct
EVGSYHAASEVCDADPYECSH

SEQ ID NO: 507 - protein, 21 aa, Artificial Sequence
  Synthetic Construct
EVGSYSDASEVCEADPYECQH

SEQ ID NO: 508 - protein, 21 aa, Artificial Sequence
  Synthetic Construct
EVGSYASASEACDLDPYECQV

SEQ ID NO: 509 - protein, 21 aa, Artificial Sequence
  Synthetic Construct
EVGSYHDASEVCEADPYECSA

SEQ ID NO: 510 - protein, 21 aa, Artificial Sequence
  Synthetic Construct
EVGSYASTPEACEADPYECSV

SEQ ID NO: 511 - protein, 21 aa, Artificial Sequence
  Synthetic Construct
EVGSYADASDVCDADPYECTV

SEQ ID NO: 512 - protein, 23 aa, Artificial Sequence
  Synthetic Construct
EVGSYYNSDDDCVSHYDPYTCYY

SEQ ID NO: 513 - protein, 23 aa, Artificial Sequence
  Synthetic Construct
EVGSYYNSDDDCISHYDPYTCYY

SEQ ID NO: 514 - protein, 23 aa, Artificial Sequence
  Synthetic Construct
EVGSYYNSDDDCASRYDPYTCYY

SEQ ID NO: 515 - protein, 23 aa, Artificial Sequence
  Synthetic Construct
EVGSYYNSDDDCVSRYDPYTCYY

SEQ ID NO: 516 - protein, 9 aa, Artificial Sequence
  Synthetic Construct
SAPLGLAAP

SEQ ID NO: 517 - protein, 9 aa, Artificial Sequence
  Synthetic Construct
RPLGLAARM

SEQ ID NO: 518 - protein, 9 aa, Artificial Sequence
  Synthetic Construct
RPLGLASRL

SEQ ID NO: 519 - protein, 9 aa, Artificial Sequence
  Synthetic Construct
PPLGLASRL

SEQ ID NO: 520 - protein, 9 aa, Artificial Sequence
  Synthetic Construct
VPLGLAAWK

SEQ ID NO: 521 - protein, 9 aa, Artificial Sequence
  Synthetic Construct
KPLGLAASL

SEQ ID NO: 522 - protein, 9 aa, Artificial Sequence
  Synthetic Construct
VPLGLASRL

SEQ ID NO: 523 - protein, 9 aa, Artificial Sequence
  Synthetic Construct
APLGLAAKL

SEQ ID NO: 524 - protein, 9 aa, Artificial Sequence
  Synthetic Construct
APLGLASWW

SEQ ID NO: 525 - protein, 9 aa, Artificial Sequence
  Synthetic Construct
IPLGLAARL

SEQ ID NO: 526 - protein, 9 aa, Artificial Sequence
  Synthetic Construct
APLGLAVML

SEQ ID NO: 527 - protein, 9 aa, Artificial Sequence
  Synthetic Construct
RPLGLAARL

SEQ ID NO: 528 - protein, 9 aa, Artificial Sequence
  Synthetic Construct
RPLGLAAAL

SEQ ID NO: 529 - protein, 9 aa, Artificial Sequence
  Synthetic Construct
RPLGLAMRL

SEQ ID NO: 530 - protein, 9 aa, Artificial Sequence
  Synthetic Construct
HPLGLASWR

SEQ ID NO: 531 - protein, 9 aa, Artificial Sequence
  Synthetic Construct
APLGLASRM

SEQ ID NO: 532 - protein, 9 aa, Artificial Sequence
  Synthetic Construct
RPLGLAAKL

SEQ ID NO: 533 - protein, 9 aa, Artificial Sequence
  Synthetic Construct
GWSGRSARP

SEQ ID NO: 534 - protein, 9 aa, Artificial Sequence
  Synthetic Construct
RSSGRSAVW

SEQ ID NO: 535 - protein, 9 aa, Artificial Sequence
  Synthetic Construct
GGSGRSAKH

SEQ ID NO: 536 - protein, 9 aa, Artificial Sequence
  Synthetic Construct
PNSGRSAKW

SEQ ID NO: 537 - protein, 9 aa, Artificial Sequence
  Synthetic Construct
PRSGRSALW

SEQ ID NO: 538 - protein, 9 aa, Artificial Sequence
  Synthetic Construct
TGSGRSAKY

SEQ ID NO: 539 - protein, 9 aa, Artificial Sequence
  Synthetic Construct
GRSGRSAVW

SEQ ID NO: 540 - protein, 9 aa, Artificial Sequence
  Synthetic Construct
ARSGRSARP

SEQ ID NO: 541 - protein, 9 aa, Artificial Sequence
  Synthetic Construct
GSGRSARPS

SEQ ID NO: 542 - protein, 9 aa, Artificial Sequence
  Synthetic Construct
RRSGRSAVW

SEQ ID NO: 543 - protein, 9 aa, Artificial Sequence
  Synthetic Construct
GPSGRSAIY

SEQ ID NO: 544 - protein, 9 aa, Artificial Sequence
  Synthetic Construct
FRSGRSAVW

SEQ ID NO: 545 - protein, 9 aa, Artificial Sequence
  Synthetic Construct
HSSGRSARY

SEQ ID NO: 546 - protein, 9 aa, Artificial Sequence
  Synthetic Construct
SNSGRSARY

SEQ ID NO: 547 - protein, 9 aa, Artificial Sequence
  Synthetic Construct
TRSGRSAVW

SEQ ID NO: 548 - protein, 9 aa, Artificial Sequence
  Synthetic Construct
SRSGRSAVW

SEQ ID NO: 549 - protein, 9 aa, Artificial Sequence
  Synthetic Construct
SMSGRSARW

SEQ ID NO: 550 - protein, 9 aa, Artificial Sequence
  Synthetic Construct
RGSGRSAVW

SEQ ID NO: 551 - protein, 9 aa, Artificial Sequence
  Synthetic Construct
SRSGRSARW

SEQ ID NO: 552 - protein, 9 aa, Artificial Sequence
  Synthetic Construct
RGSGRSARH

SEQ ID NO: 553 - protein, 9 aa, Artificial Sequence
  Synthetic Construct
ARSGRSARW

SEQ ID NO: 554 - protein, 9 aa, Artificial Sequence
  Synthetic Construct
SRSGRSAVY

SEQ ID NO: 555 - protein, 9 aa, Artificial Sequence
  Synthetic Construct
RHSGRSAVY

SEQ ID NO: 556 - protein, 5 aa, Artificial Sequence
  Synthetic Construct
SYNMH

SEQ ID NO: 557 - protein, 17 aa, Artificial Sequence
  Synthetic Construct
AIYPGSGDTSYNQKFKG

SEQ ID NO: 558 - protein, 12 aa, Artificial Sequence
  Synthetic Construct
STYYGGDWYFNV

SEQ ID NO: 559 - protein, 10 aa, Artificial Sequence
  Synthetic Construct
RASSSVSYIH

SEQ ID NO: 560 - protein, 7 aa, Artificial Sequence
  Synthetic Construct
ATSNLAS

SEQ ID NO: 561 - protein, 9 aa, Artificial Sequence
  Synthetic Construct
QQWTSNPPT

SEQ ID NO: 562 - protein, 121 aa, Artificial Sequence
  Synthetic Construct
EVQLVQSGAEVKKPGSSVKVSCKASGYTFTSYNMHWVRQAPGKGLEWIGAIYPGSGDTSYNQKFKGRVTITADKSTSTAYMELSSLRSEDTAVYYCARSTYYGGDWYFNVWGQGTLVTVSS

SEQ ID NO: 563 - protein, 106 aa, Artificial Sequence
  Synthetic Construct
QIVLSQSPAILSASPGEKVTMTCRASSSVSYIHWFQQKPGKSPKPWIYATSNLASGVPVRFSGSGSGTSYSLTISRVEAEDAATYYCQQWTSNPPTFGGGTKVEIK

SEQ ID NO: 564 - protein, 213 aa, Artificial Sequence
  Synthetic Construct
QIVLSQSPAILSASPGEKVTMTCRASSSVSYIHWFQQKPGKSPKPWIYATSNLASGVPVRFSGSGSGTSYSLTISRVEAEDAATYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 565 - protein, 451 aa, Artificial Sequence
  Synthetic Construct
EVQLVQSGAEVKKPGSSVKVSCKASGYTFTSYNMHWVRQAPGKGLEWIGAIYPGSGDTSYNQKFKGRVTITADKSTSTAYMELSSLRSEDTAVYYCARSTYYGGDWYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVKLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 566 - protein, 486 aa, Artificial Sequence; identical to SEQ ID NO: 428
  Synthetic Construct

SEQ ID NO: 567 - protein, 518 aa, Artificial Sequence
  Synthetic Construct
EVGSYPYDDPDCPSHESDCDQGGGPLGLAGGSQAVVTQEPSLTVSPGGTVTLTCGSSTGAVTTSNYPNWVQQKPGQAPRGLIGGTNKRAPGVPARFSGSLLGGKAALTLSGAQPEDEAEYYCALWYSNLWVFGGGTKLTVLGGGGSGGGGSGGGGSGGGGSEVQLVESGGGLVQPGGSLRLSCAASGFTFNTYAINWVRQAPGKGLEWVGRIRSKYNNYATYYAESVKGRFTISRDDSKNTLYLQINSLRAEDTAVYYCVRHGNFGTSYVSWFAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 568 - protein, 486 aa, Artificial Sequence; identical to SEQ ID NO: 426
  Synthetic Construct

SEQ ID NO: 569 - protein, 518 aa, Artificial Sequence; identical to SEQ ID NO: 430
  Synthetic Construct

SEQ ID NO: 570 - protein, 213 aa, Artificial Sequence
  Synthetic Construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 571 - protein, 452 aa, Artificial Sequence
  Synthetic Construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 572 - protein, 219 aa, Artificial Sequence
  Synthetic Construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 573 - protein, 449 aa, Artificial Sequence
  Synthetic Construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 574 - protein, 213 aa, Artificial Sequence
  Synthetic Construct
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 575 - protein, 450 aa, Artificial Sequence
  Synthetic Construct
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 576 - protein, 485 aa, Artificial Sequence
  Synthetic Construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 577 - protein, 254 aa, Artificial Sequence
  Synthetic Construct
EVGSYYDNYNDCDNYDDDCYYSGRSAGGGGTPLGLAGSGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 578 - protein, 446 aa, Artificial Sequence
  Synthetic Construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 579 - protein, 254 aa, Artificial Sequence
  Synthetic Construct
EVGSYNADYHQCSDVPTDCLDSGRSAGGGGTPLGLAGSGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 580 - protein, 446 aa, Artificial Sequence; identical to SEQ ID NO: 578
  Synthetic Construct

SEQ ID NO: 581 - protein, 254 aa, Artificial Sequence
  Synthetic Construct
EVGSYDSYDYNCYHDHHTCHDSGRSAGGGGTPLGLAGSGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 582 - protein, 446 aa, Artificial Sequence; identical to SEQ ID NO: 578
  Synthetic Construct

SEQ ID NO: 583 - protein, 254 aa, Artificial Sequence
  Synthetic Construct
EVGSYAYHDDDCPDDDYDCASSGRSAGGGGTPLGLAGSGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 584 - protein, 446 aa, Artificial Sequence; identical to SEQ ID NO: 578
  Synthetic Construct

SEQ ID NO: 585 - protein, 21 aa, Artificial Sequence
  Synthetic Construct
EVGSYYDNYNDCDNYDDDCYY

SEQ ID NO: 586 - protein, 21 aa, Artificial Sequence
  Synthetic Construct
EVGSYNADYHQCSDVPTDCLD

SEQ ID NO: 587 - protein, 21 aa, Artificial Sequence
  Synthetic Construct
EVGSYDSYDYNCYHDHHTCHD

SEQ ID NO: 588 - protein, 21 aa, Artificial Sequence
  Synthetic Construct
EVGSYAYHDDDCPDDDYDCAS

SEQ ID NO: 589 - protein, 257 aa, Artificial Sequence
  Synthetic Construct
EVGSYDDPDFPCDPDDADCPNSGRSAGGGGTPLGLAGSGGSQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 590 - protein, 455 aa, Artificial Sequence
  Synthetic Construct
EVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 591 - protein, 257 aa, Artificial Sequence
  Synthetic Construct
EVGSYAPHDPDCPADPPSCYPSGRSAGGGGTPLGLAGSGGSQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 592 - protein, 455 aa, Artificial Sequence; identical to SEQ ID NO: 590
  Synthetic Construct

SEQ ID NO: 593 - protein, 257 aa, Artificial Sequence
  Synthetic Construct
EVGSYPYDDPDCPSHDSDCDNSGRSAGGGGTPLGLAGSGGSQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 594 - protein, 455 aa, Artificial Sequence; identical to SEQ ID NO: 590
  Synthetic Construct

SEQ ID NO: 595 - protein, 257 aa, Artificial Sequence
  Synthetic Construct
EVGSYDADDPDCPADNNHCHYSGRSAGGGGTPLGLAGSGGSQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 596 - protein, 455 aa, Artificial Sequence; identical to SEQ ID NO: 590
  Synthetic Construct

SEQ ID NO: 597 - protein, 21 aa, Artificial Sequence
  Synthetic Construct
EVGSYDDPDFPCDPDDADCPN

SEQ ID NO: 598 - protein, 21 aa, Artificial Sequence
  Synthetic Construct
EVGSYAPHDPDCPADPPSCYP

SEQ ID NO: 599 - protein, 21 aa, Artificial Sequence
  Synthetic Construct
EVGSYDADDPDCPADNNHCHY

SEQ ID NO: 600 - protein, 5 aa, Artificial Sequence
  Synthetic Construct
TYAMT

SEQ ID NO: 601 - protein, 5 aa, Artificial Sequence
  Synthetic Construct
DYAMN

SEQ ID NO: 602 - protein, 5 aa, Artificial Sequence
  Synthetic Construct
TYALN

SEQ ID NO: 603 - protein, 19 aa, Artificial Sequence
  Synthetic Construct
RIRSKYNNYATYYADTVKG

SEQ ID NO: 604 - protein, 14 aa, Artificial Sequence
  Synthetic Construct
HGNFGNSYVSYWAY

SEQ ID NO: 605 - protein, 14 aa, Artificial Sequence
  Synthetic Construct
HGNYGNSYVSWFAY

SEQ ID NO: 606 - protein, 14 aa, Artificial Sequence
  Synthetic Construct
RSSTGAVTTSNYPN

SEQ ID NO: 607 - protein, 14 aa, Artificial Sequence
  Synthetic Construct
ASSTGAVTTSNYAN

SEQ ID NO: 608 - protein, 14 aa, Artificial Sequence
  Synthetic Construct
GSSTGAVTSGNYAN

SEQ ID NO: 609 - protein, 14 aa, Artificial Sequence
  Synthetic Construct
GSSTGAVTTSNYVN

SEQ ID NO: 610 - protein, 9 aa, Artificial Sequence
  Synthetic Construct
ALWYSNRWV

SEQ ID NO: 611 - protein, 125 aa, Artificial Sequence
  Synthetic Construct
EVQLVESGGGLVKPGGSLRLSCAASGFTFSTYAMNWVRQAPGKGLEWVSRIRSKYNNYATYYADSVKGRFTISRDDSKNTLYLQINSLRAEDTAVYYCVRHGNFGTSYVSWFAYWGQGTLVTVSS

SEQ ID NO: 612 - protein, 125 aa, Artificial Sequence
  Synthetic Construct
EVQLVESGGGLVKPGGSLRLSCAASGFTFSTYAMNWVRQAPGKGLEWVSRIRSKYNNYATYYADSVKDRFTISRDDSKNTLYLQINSLRAEDTAVYYCVRHGNFGNSYVSYWAYWGQGTLVTVSS

SEQ ID NO: 613 - protein, 125 aa, Artificial Sequence
  Synthetic Construct
EVQLVESGGGLVKPGGSLRLSCAASGFTFNTYAMNWVRQAPGKGLEWVGRIRSKYNNYATYYADSVKGRFTISRDDSKNTLYLQINSLRAEDTAVYYCVRHGNFGNSYVSWFAYWGQGTLVTVSS

SEQ ID NO: 614 - protein, 125 aa, Artificial Sequence
  Synthetic Construct
EVQLVESGGGLVQPGGSLRLSCAASGFTFNSYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 615 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADSVKSRFTISRDDSKNTLYLQMNSLRAEDTAVYYCVRHGNFGNSYVSWFAYWGQGTLVTVSS

SEQ ID NO: 616 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADSVKGRFTISRDDSRSTLYLQMNSLRAEDTAVYYCVRHGNFGNSYVSWFAYWGQGTLVTVSS

SEQ ID NO: 617 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADSVKGRFTISRDDSQSTLYLQMNSLRAEDTAVYYCVRHGNFGNSYVSWFAYWGQGTLVTVSS

SEQ ID NO: 618 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADSVKGRFTISRDDSKNSIYLQMNSLRAEDTAVYYCVRHGNFGNSYVSWFAYWGQGTLVTVSS

SEQ ID NO: 619 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADSVKGRFTISRDDSKNTLYLQMNTLRAEDTAVYYCVRHGNFGNSYVSWFAYWGQGTLVTVSS

SEQ ID NO: 620 - protein, 125 aa, Artificial Sequence
  Synthetic Construct
EVQLVETGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 621 - protein, 125 aa, Artificial Sequence
  Synthetic Construct
EVQLVESGGGLIQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 622 - protein, 125 aa, Artificial Sequence
  Synthetic Construct
EVQLVESGGGLLQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 623 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSS

SEQ ID NO: 624 - protein, 125 aa, Artificial Sequence
  Synthetic Construct
EVQLVESGGGLVQPGGSLRLSCAASGFTFNTYAMTWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 625 - protein, 125 aa, Artificial Sequence
  Synthetic Construct
EVQLVESGGGLVQPGGSLRLSCAASGFTFNTYAMNWVRQAPGQGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 626 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADTVKGRFTISRDDSKNTLYLQMNSLRAEDTAVYYCVRHGNFGNSYVSWFAYWGQGTLVTVSS

SEQ ID NO: 627 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNYGNSYVSWFAYWGQGTLVTVSS

SEQ ID NO: 628 - protein, 125 aa, Artificial Sequence
  Synthetic Construct
EVQLVESGGGLVQPGGSLRLSCAASGFTFSSYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 629 - protein, 125 aa, Artificial Sequence
  Synthetic Construct
EVQLVESGGGLVQPGGSLRLSCAASGFTFSDYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 630 - protein, 125 aa, Artificial Sequence
  Synthetic Construct
EVQLVESGGGLVQPGGSLRLSCAASGFTFNTYAINWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 631 - protein, 125 aa, Artificial Sequence
  Synthetic Construct
EVQLVESGGGLVQPGGSLRLSCAASGFTFNTYALNWVRQAPGKGLEWVGRIRSKYNNYATYYADSVKGRFTISRDDSKNTLYLQMNSLRAEDTAVYYCVRHGNFGNSYVSWFAYWGQGTLVTVSS

SEQ ID NO: 632 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADSVKGRFTISRDDSKNTLYLQINSLRAEDTAVYYCVRHGNFGNSYVSWFAYWGQGTLVTVSS

SEQ ID NO: 633 - protein, 125 aa, Artificial Sequence
  Synthetic Construct
EVQLVESGGGLVQPGGSLRLSCAASGFTFNTYAMNWVRQAPGKGLEWVGRIRSKYNNYATYYADSVKGRFTISRDDSKNTLYLQLNSLRAEDTAVYYCVRHGNFGNSYVSWFAYWGQGTLVTVSS

SEQ ID NO: 634 - protein, 125 aa, Artificial Sequence
  Synthetic Construct
EVQLVESGGGLVQPGGSLRLSCAASGFTFSTYAINWVRQAPGKGLEWVSRIRSKYNNYATYYADSVKDRFTISRDDSKNTLYLQINSLRAEDTAVYYCVRHGNFGTSYVSWFAYWGQGTLVTVSS

SEQ ID NO: 635 - protein, 125 aa, Artificial Sequence
  Synthetic Construct
EVQLVESGGGLVKPGGSLRLSCAASGFTFSTYAINWVRQAPGKGLEWVSRIRSKYNNYATYYADSVKDRFTISRDDSKNTLYLQINSLRAEDTAVYYCVRHGNFGNSYVSYWAYWGQGTLVTVSS

SEQ ID NO: 636 - protein, 125 aa, Artificial Sequence
  Synthetic Construct
EVQLVESGGGLVKPGGSLRLSCAASGFTFSTYAINWVRQAPGKGLEWVSRIRSKYNNYATYYAESVKDRFTISRDDSKNTLYLQINSLRAEDTAVYYCVRHGNFGNSYVSYWAYWGQGTLVTVSS

SEQ ID NO: 637 - protein, 125 aa, Artificial Sequence
  Synthetic Construct
EVQLVESGGGLVKPGGSLRLSCAASGFTFSTYAINWVRQAPGKGLEWVSRIRSKYNNYATYYADSVKGRFTISRDDSKNTLYLQINSLRAEDTAVYYCVRHGNFGTSYVSWFAYWGQGTLVTVSS

SEQ ID NO: 638 - protein, 125 aa, Artificial Sequence
  Synthetic Construct
EVQLVESGGGLVKPGGSLRLSCAASGFTFSTYAINWVRQAPGKGLEWVSRIRSKYNNYATYYAESVKGRFTISRDDSKNTLYLQINSLRAEDTAVYYCVRHGNFGTSYVSWFAYWGQGTLVTVSS

SEQ ID NO: 639 - protein, 125 aa, Artificial Sequence
  Synthetic Construct
EVQLVESGGGLVKPGGSLRLSCAASGFTFSTYAINWVRQAPGKGLEWVSRIRSKYNNYATYYADSVKDRFTISRDDSKNTLYLQINSLRAEDTAVYYCVRHGNFGTSYVSWFAYWGQGTLVTVSS

SEQ ID NO: 640 - protein, 125 aa, Artificial Sequence
  Synthetic Construct
EVQLVESGGGLVKPGGSLRLSCAASGFTFSTYAINWVRQAPGKGLEWVSRIRSKYNNYATYYAESVKDRFTISRDDSKNTLYLQINSLRAEDTAVYYCVRHGNFGTSYVSWFAYWGQGTLVTVSS

SEQ ID NO: 641 - protein, 109 aa, Artificial Sequence
  Synthetic Construct
EAVVTQEPSLTVSPGGTVTLTCRSSTGAVTTSNYPNWVQQKPGQAPRGLIGGTKFRAPGTPARFSGSLLGGKAALTLSGAQPEDEAEYYCALWYSTLWVFGGGTKLTVL

SEQ ID NO: 642 - protein, 109 aa, Artificial Sequence
  Synthetic Construct
EAVVTQEPSLTVSPGGTVTLTCGSSTGAVTTSNYANWVQQKPGQAPRGLIGGTNKRAPGTPARFSGSLLGGKAALTLSGAQPEDEAEYYCALWYSNLWVFGGGTKLTVL

SEQ ID NO: 643 - protein, 109 aa, Artificial Sequence
  Synthetic Construct
EAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 644 - protein, 109 aa, Artificial Sequence
  Synthetic Construct
QTVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNRWVFGGGTKLTVL

SEQ ID NO: 645 - protein, 109 aa, Artificial Sequence
  Synthetic Construct
QAVVTQESALTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 646 - protein, 109 aa, Artificial Sequence
  Synthetic Construct
QAVVTQESTLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 647 - protein, 109 aa, Artificial Sequence
  Synthetic Construct
QAVVTQEPSLSTSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 648 - protein, 109 aa, Artificial Sequence
  Synthetic Construct
QAVVTQEPSLSASPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 649 - protein, 109 aa, Artificial Sequence
  Synthetic Construct
QAVVTQEPSLTVSPGVTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 650 - protein, 109 aa, Artificial Sequence
  Synthetic Construct
QAVVTQEPSLTVSPGGTVTLTCRSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 651 - protein, 109 aa, Artificial Sequence
  Synthetic Construct
QAVVTQEPSLTVSPGGTVTLTCASSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 652 - protein, 109 aa, Artificial Sequence
  Synthetic Construct
QAVVTQEPSLTVSPGGTVTLTCGSSTGAVTSGNYANWVQQKPGQAPRGLIGGTNKRAPGVPARFSGSLLGGKAALTLSGAQPEDEAEYYCALWYSNLWVFGGGTKLTVL

SEQ ID NO: 653 - protein, 109 aa, Artificial Sequence
  Synthetic Construct
QAVVTQEPSLTVSPGGTVTLTCGSSTGAVTTSNYVNWVQQKPGQAPRGLIGGTNKRAPGVPARFSGSLLGGKAALTLSGAQPEDEAEYYCALWYSNLWVFGGGTKLTVL

SEQ ID NO: 654 - protein, 109 aa, Artificial Sequence
  Synthetic Construct
QAVVTQEPSLTVSPGGTVTLTCGSSTGAVTTSNYANWVQQKPDHLFRGLIGGTNKRAPGVPARFSGSLLGGKAALTLSGAQPEDEAEYYCALWYSNLWVFGGGTKLTVL

SEQ ID NO: 655 - protein, 109 aa, Artificial Sequence
  Synthetic Construct
QAVVTQEPSLTVSPGGTVTLTCGSSTGAVTTSNYANWVQQKPGQAPRGLIGGTNKRAPWTPARFSGSLLGGKAALTLSGAQPEDEAEYYCALWYSNLWVFGGGTKLTVL

SEQ ID NO: 656 - protein, 109 aa, Artificial Sequence
  Synthetic Construct
QAVVTQEPSLTVSPGGTVTLTCGSSTGAVTTSNYANWVQQKPGQAPRGLIGGTNKRAPWSPARFSGSLLGGKAALTLSGAQPEDEAEYYCALWYSNLWVFGGGTKLTVL

SEQ ID NO: 657 - protein, 109 aa, Artificial Sequence
  Synthetic Construct
QAVVTQEPSLTVSPGGTVTLTCGSSTGAVTTSNYANWVQQKPGQAPRGLIGGTNKRAPGVPARFSGSLIGGKAALTLSGAQPEDEAEYYCALWYSNLWVFGGGTKLTVL

SEQ ID NO: 658 - protein, 109 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGDKAALTLSGAQPEDEAEYYCALWYSNLWVFGGGTKLTVL

SEQ ID NO: 659 - protein, 109 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGNKAALTLSGAQPEDEAEYYCALWYSNLWVFGGGTKLTVL

SEQ ID NO: 660 - protein, 109 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGGKAALTITGAQPEDEAEYYCALWYSNLWVFGGGTKLTVL

SEQ ID NO: 661 - protein, 109 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGGKAALTLSGVQPEDEAEYYCALWYSNLWVFGGGTKLTVL

SEQ ID NO: 662 - protein, 109 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGGKAALTLSGAQPEDESDYYCALWYSNLWVFGGGTKLTVL

SEQ ID NO: 663 - protein, 109 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGGKAALTLSGAQPEDESQYYCALWYSNLWVFGGGTKLTVL

SEQ ID NO: 664 - protein, 109 aa, Artificial Sequence
  Synthetic Construct
QAVVTQEPSLTVSPGGTVTLTCGSSTGAVTTSNYANWVQQKPGQAPRGLIGGTNKRAPGVPARFSGSIIGGKAALTLSGAQPEDEAEYYCALWYSNLWVFGGGTKLTVL

SEQ ID NO: 665 - protein, 109 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGGKAALTLSGAQADDEAEYYCALWYSNLWVFGGGTKLTVL

SEQ ID NO: 666 - protein, 109 aa, Artificial Sequence
  Synthetic Construct
QAVVTQEPSLTVSPGGTVTLTCGSSTGAVTTSNYANWVQQKPGEAPRGLIGGTNKRAPGVPARFSGSLLGGKAALTLSGAQPEDEAEYYCALWYSNLWVFGGGTKLTVL

SEQ ID NO: 667 - protein, 5 aa, Artificial Sequence
  Synthetic Construct
EVGSY

SEQ ID NO: 668 - protein, 16 aa, Artificial Sequence
  Synthetic Construct
  VARIANT 11: Xaa = D or E
  VARIANT 16: Xaa = N or Q
PYDDPDCPSHXSDCDX

SEQ ID NO: 669 - protein, 16 aa, Artificial Sequence
  Synthetic Construct
  VARIANT 1: Xaa = A or D
  VARIANT 2: Xaa = A or D or P
  VARIANT 3: Xaa = D or H or P
  VARIANT 5: Xaa = F or P
  VARIANT 6: Xaa = D or P
  VARIANT 8: Xaa = D or P
  VARIANT 9: Xaa = A or P
  VARIANT 11: Xaa = D or N or P
  VARIANT 12: Xaa =  A or N or P
  VARIANT 13: Xaa = D or H or S
  VARIANT 15: Xaa = H or P or Y
  VARIANT 16: Xaa = N or P or Y
XXXDXXCXXDXXXCXX

SEQ ID NO: 670 - protein, 14 aa, Artificial Sequence
  Synthetic Construct
  VARIANT 3, 4, 6, 7, 11, 14: Xaa = D or E
  VARIANT 4: Xaa = A or F or V or Y
  VARIANT 6: Xaa = D or E
  VARIANT 7: Xaa = A or L
  VARIANT 11: Xaa = D or E
  VARIANT 14: Xaa = A or F or Y
ESXXCXXDPFXCQX

SEQ ID NO: 671 - protein, 16 aa, Artificial Sequence
  Synthetic Construct
  VARIANT 1: Xaa = A or H or S
  VARIANT 2: Xaa = A or D or S
  VARIANT 3: Xaa = A or T or V
  VARIANT 4: Xaa = P or S or T
  VARIANT 5: Xaa = D or E
  VARIANT 6: Xaa = A or V
  VARIANT 8: Xaa = D or E
  VARIANT 9: Xaa = A or L
  VARIANT 15: Xaa = Q or S or T
  VARIANT 16: Xaa = A or H or V
XXXXXXCXXDPYECXX

SEQ ID NO: 672 - protein, 18 aa, Artificial Sequence
  Synthetic Construct
  VARIANT 8: Xaa = A or I or V
  VARIANT 10: Xaa = H or R
YNSDDDCXSXYDPYTCYY

SEQ ID NO: 673 - protein, 486 aa, Artificial Sequence
  Synthetic Construct
EAVVTQEPSLTVSPGGTVTLTCRSSTGAVTTSNYANWVQQKPGQAPRGLIGGTKFRAPGVPSRFSGSLLGGKAALTLSGAQPEDEAEYYCALWYSDHWVFGGGTKLTVLGGGGSGGGGSGGGGSGGGGSEVQLVESGGGLVQPGGSLRLSCAASGFTFSSYAINWVRQAPGKGLEWVGRIRSKYNNYATYYADSVKGRFTISRDDSKNTLYLQINSLRAEDTAVYYCARHGNFGTSYVSYWAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 674 - protein, 486 aa, Artificial Sequence
  Synthetic Construct
QAVVTQEPSLTVSPGGTVTLTCPSSTGAVTTSNYANWVQQKPGQAPRGLIGGTKFRAPGTPARFSGSLLGGKAALTLSGAQPEDEAEYYCALWYSDHWVFGGGTKLTVLGGGGSGGGGSGGGGSGGGGSEVQLVESGGGLVKPGGSLRLSCAASGFTFSSYAINWVRQAPGKGLEWVGRIRSKYNNYATYYADSVKDRFTISRDDSKNTLYLQLNSLRAEDTAVYYCVRHGNFGTSYVSYWAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 675 - protein, 486 aa, Artificial Sequence
  Synthetic Construct
QAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNHWVFGGGTKLTVLGGGGSGGGGSGGGGSGGGGSEVQLVESGGGLVQPGGSLRLSCAASGFTFSSYAINWVRQAPGKGLEWVGRIRSKYNNYATYYAESVKGRFTISRDDSKNTLYLQINSLRAEDTAVYYCVRHGNYGTSYVSYWAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 676 - protein, 486 aa, Artificial Sequence
  Synthetic Construct
EAVVTQEPSLTVSPGGTVTLTCGSSTGAVTTSNYANWVQQKPGQAPRGLIGGTKFRAPGIPARFSGSLLGGKAALTLSGAQPEDEAEYYCALWYSDHWVFGGGTKLTVLGGGGSGGGGSGGGGSGGGGSEVQLVESGGGLVQPGGSLRLSCAASGFTFSTYAINWVRQAPGKGLEWVGRIRSKYNNYATYYAESVKGRFTISRDDSKNTLYLQINSLRAEDTAVYYCARHGNFGTSYVSWFAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 677 - protein, 486 aa, Artificial Sequence
  Synthetic Construct
QAVVTQEPSLTVSPGGTVTLTCGSSTGAVTTSNYPNWVQQKPGQAPRGLIGGTNKRAPGVPARFSGSLLGGKAALTLSGAQPEDEAEYYCALWYSNLWVFGGGTKLTVLGGGGSGGGGSGGGGSGGGGSEVQLVESGGGLVKPGGSLRLSCAASGFTFSSYAINWVRQAPGKGLEWVGRIRSKYNNYATYYAESVKGRFTISRDDSKNTLYLQINSLRAEDTAVYYCVRHGNFGNSYVSYWAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 678 - protein, 486 aa, Artificial Sequence
  Synthetic Construct
EAVVTQEPSLTVSPGGTVTLTCGSSTGAVTTSNYPNWVQQKPGQAPRGLIGGTKFRAPGIPARFSGSLLGGKAALTLSGAQPEDEAEYYCALWYSNHWVFGGGTKLTVLGGGGSGGGGSGGGGSGGGGSEVQLVESGGGLVQPGGSLRLSCAASGFTFSTYAINWVRQAPGKGLEWVSRIRSKYNNYATYYAESVKGRFTISRDDSKNTLYLQINSLRAEDTAVYYCVRHGNFGTSYVSYWAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 679 - protein, 486 aa, Artificial Sequence
  Synthetic Construct
EAVVTQEPSLTVSPGGTVTLTCGSSTGAVTTSNYANWVQQKPGQAPRGLIGGTKFRAPGAPARFSGSLLGGKAALTLSGAQPEDEAEYYCALWYSNHWVFGGGTKLTVLGGGGSGGGGSGGGGSGGGGSEVQLVESGGGLVKPGGSLRLSCAASGFTFSTYAINWVRQAPGKGLEWVGRIRSKYNNYATYYADSVKGRFTISRDNSKNTLYLQLNSLRAEDTAVYYCARHGNFGNSYVSYWAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 680 - protein, 486 aa, Artificial Sequence
  Synthetic Construct
QAVVTQEPSLTVSPGGTVTLTCGSSTGAVTTSNYPNWVQQKPGQAPRGLIGGTKFRAPGAPARFSGSLLGGKAALTLSGAQPEDEAEYYCALWYSDHWVFGGGTKLTVLGGGGSGGGGSGGGGSGGGGSEVQLVESGGGLVQPGGSLRLSCAASGFTFSSYAINWVRQAPGKGLEWVGRIRSKYNNYATYYADSVKDRFTISRDNSKNTLYLQLNSLRAEDTAVYYCVRHGNFGNSYVSYWAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 681 - protein, 486 aa, Artificial Sequence
  Synthetic Construct
EAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSDLWVFGGGTKLTVLGGGGSGGGGSGGGGSGGGGSEVQLVESGGGLVKPGGSLRLSCAASGFTFSSYAINWVRQAPGKGLEWVGRIRSKYNNYATYYADSVKGRFTISRDDSKNTLYLQINSLRAEDTAVYYCVRHGNYGTSYVSYWAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 682 - protein, 486 aa, Artificial Sequence
  Synthetic Construct
EAVVTQEPSLTVSPGGTVTLTCASSTGAVTTSNYPNWVQQKPGQAPRGLIGGTKFRAPGVPARFSGSLLGGKAALTLSGAQPEDEAEYYCALWYSNHWVFGGGTKLTVLGGGGSGGGGSGGGGSGGGGSEVQLVESGGGLVQPGGSLRLSCAASGFTFSSYAIHWVRQAPGKGLEWVGRIRSKYNNYATYYAESVKDRFTISRDDSKNTLYLQLNSLRAEDTAVYYCARHGNFGNSYVSYWAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPGK

SEQ ID NO: 683 - protein, 109 aa, Artificial Sequence
  Synthetic Construct
EAVVTQEPSLTVSPGGTVTLTCRSSTGAVTTSNYANWVQQKPGQAPRGLIGGTKFRAPGVPSRFSGSLLGGKAALTLSGAQPEDEAEYYCALWYSDHWVFGGGTKLTVL

SEQ ID NO: 684 - protein, 125 aa, Artificial Sequence
  Synthetic Construct
EVQLVESGGGLVQPGGSLRLSCAASGFTFSSYAINWVRQAPGKGLEWVGRIRSKYNNYATYYADSVKGRFTISRDDSKNTLYLQINSLRAEDTAVYYCARHGNFGTSYVSYWAYWGQGTLVTVSS

SEQ ID NO: 685 - protein, 109 aa, Artificial Sequence
  Synthetic Construct
QAVVTQEPSLTVSPGGTVTLTCPSSTGAVTTSNYANWVQQKPGQAPRGLIGGTKFRAPGTPARFSGSLLGGKAALTLSGAQPEDEAEYYCALWYSDHWVFGGGTKLTVL

SEQ ID NO: 686 - protein, 125 aa, Artificial Sequence
  Synthetic Construct
EVQLVESGGGLVKPGGSLRLSCAASGFTFSSYAINWVRQAPGKGLEWVGRIRSKYNNYATYYADSVKDRFTISRDDSKNTLYLQLNSLRAEDTAVYYCVRHGNFGTSYVSYWAYWGQGTLVTVSS

SEQ ID NO: 687 - protein, 9 aa, Artificial Sequence
  Synthetic Construct
ALWYSDHWV

SEQ ID NO: 688 - protein, 5 aa, Artificial Sequence
  Synthetic Construct
SYAIN

SEQ ID NO: 689 - protein, 14 aa, Artificial Sequence
  Synthetic Construct
HGNFGTSYVSYWAY

SEQ ID NO: 690 - protein, 14 aa, Artificial Sequence
  Synthetic Construct
PSSTGAVTTSNYAN

SEQ ID NO: 691 - protein, 9 aa, Artificial Sequence
  Synthetic Construct
ALWYSNHWV

SEQ ID NO: 692 - protein, 14 aa, Artificial Sequence
  Synthetic Construct
HGNYGTSYVSYWAY

SEQ ID NO: 693 - protein, 18 aa, Artificial Sequence
  Synthetic Construct
RIRSKYNNYATYYADSVK

SEQ ID NO: 694 - protein, 14 aa, Artificial Sequence
  Synthetic Construct
ASSTGAVTTSNYPN

SEQ ID NO: 695 - protein, 5 aa, Artificial Sequence
  Synthetic Construct
SYAIH

SEQ ID NO: 696 - protein, 18 aa, Artificial Sequence
  Synthetic Construct
RIRSKYNNYATYYAESVK

SEQ ID NO: 697 - protein, 214 aa, Artificial Sequence; identical to SEQ ID NO: 112
  Synthetic Construct

SEQ ID NO: 698 - protein, 450 aa, Artificial Sequence; identical to SEQ ID NO: 84
  Synthetic Construct

SEQ ID NO: 699 - protein, 520 aa, Artificial Sequence; identical to SEQ ID NO: 85
  Synthetic Construct

SEQ ID NO: 700 - protein, 7 aa, Artificial Sequence
  Synthetic sequence
TGGVGVG

SEQ ID NO: 701 - protein, 16 aa, Artificial Sequence
  Synthetic sequence
LIDWADDKYYSPSLKS

SEQ ID NO: 702 - protein, 13 aa, Artificial Sequence
  Synthetic sequence
GGSDTVIGDWFAY

SEQ ID NO: 703 - protein, 11 aa, Artificial Sequence
  Synthetic sequence
RASQSIGSYLA

SEQ ID NO: 704 - protein, 7 aa, Artificial Sequence
  Synthetic sequence
DASNLET

SEQ ID NO: 705 - protein, 8 aa, Artificial Sequence
  Synthetic sequence
QQGYYLWT

SEQ ID NO: 706 - protein, 123 aa, Artificial Sequence
  Synthetic sequence
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSS

SEQ ID NO: 707 - protein, 107 aa, Artificial Sequence
  Synthetic sequence
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 708 - protein, 115 aa, Artificial Sequence
  Synthetic sequence
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSS

SEQ ID NO: 709 - protein, 113 aa, Artificial Sequence
  Synthetic sequence
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKR

SEQ ID NO: 710 - protein, 449 aa, Artificial Sequence
  Synthetic sequence
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 711 - protein, 213 aa, Artificial Sequence
  Synthetic sequence
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0326860 A1
(43) Pub. Date: Oct. 23, 2025

(54) ANTI-CD3 ANTIBODIES AND METHODS OF USE THEREOF

(71) Applicant: ADAGENE PTE. LTD., Singapore (SG)

(72) Inventors: Fangyong DU, Suzhou Industrial Park (CN); Peter Peizhi luo, Morgan Hill, CA (US); Yan LI, Suzhou, Jiangsu Park (CN); Guizhong LIU, Morgan HILL, CA (US)

(73) Assignee: ADAGENE PTE. LTD., Singapore (SG)

(21) Appl. No.: 18/546,246

(22) PCT Filed: Feb. 11, 2022
(86) PCT No.: PCT/CN2022/076000

§ 371 Date: Aug. 11, 2023

(30) Foreign Application Priority Data
Feb. 11, 2021 (WO) PCT/CN2021/076626
Jul. 28, 2021 (WO) PCT/CN2021/109057

(51) Int. Cl. C07K 16/32; C07K 16/28

(52) U.S. Cl. CPC C07K 16/32; C07K 16/2809; C07K 16/2887; C07K 2317/31; C07K 2317/92; C07K 2319/50

(57) ABSTRACT

The present application provides antibodies comprising an antigen-binding fragment of an anti-CD3 antibody having tailor-made affinity to CD3 in low to medium range. In some embodiments, the antibody further comprises an antigen-binding fragment that specifically binds to a target antigen, such as HER2, CD20, TROP2, BCMA, or CD19. Also provided are anti-CD3 antibodies, masked anti-CD3 antibodies (including activatable anti-CD3 antibodies), anti-CD20 antibodies, and masked anti-HER2 antibodies (including activatable anti-HER2 antibodies). The antibodies described herein are useful for treatment of cancer.

### CROSS REFERENCE TO RELATED APPLICATIONS

This application is a national stage application under 35 U.S.C. § 371 of International Patent Application No. PCT/CN2022/076000, filed Feb. 11, 2022, which claims priority benefit of International Patent Application Nos. PCT/CN2021/076626 filed Feb. 11, 2021, and PCT/CN2021/109057 filed Jul. 28, 2021, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 695402000900SUBSEQLIST.TXT, date recorded: Jan. 3, 2024, size: 432,924 bytes).

### FIELD

The present application relates to antibodies targeting CD3, including multispecific antibodies targeting CD3, masked and activatable antibodies targeting CD3, methods of preparation, and methods of use thereof.

### BACKGROUND

Bispecific T-cell engager antibodies (BiTEs or TCEs) have been explored as a means to recruit cytolytic T-cells to kill tumor cells. This is based on the simultaneous recognition of an antigen on tumor cells and binding to the CD3 epsilon chain, or CD3, within the T-cell receptor complex on T-cells that bridges malignant tumor cells directly to CD3+ T-cells. Blinatumomab, or BLINCYTO®, the first bispecific T-cell engager reactive with the B-cell antigen CD19, was approved by the FDA in 2014 for the treatment of neoplasms. While early studies showed promising clinical efficacy, bispecific T-cell engagers were hampered by severe dose-limiting toxicities primarily manifesting as cytokine release syndrome, which resulted in a prohibitively narrow therapeutic window. There is a need for activatable BiTE or TCE molecules with enhanced specificity and reduced side effects.

An activatable antibody, also known as a SAFEBODY™, is designed to mask an antigen-binding interface with a masking motif, which then prevents an antibody from binding to its target in healthy tissues. The masking motif is designed to activate, or unmask, the antibody to allow binding in the tumor microenvironment (“TME”) where certain activation conditions such as a protease is upregulated or favorable competition via highly localized antigen concentration as compared to healthy tissues, allowing the antibody to bind to its target for tumor killing. See, for example, WO2019/149282. Activatable antibodies thus provide antigen-specific binding proteins that are activated predominantly in the TME while remaining largely in an inactive state in healthy tissues.

### BRIEF SUMMARY

The present application provides multispecific antibodies targeting CD3 and another target antigen (e.g., HER2, CD20, TROP2, BCMA, or CD19), masked antibodies (including activatable antibodies such as activatable multispecific antibodies), isolated anti-CD3 antibodies, masked (e.g., activatable) antibodies targeting HER2, and methods of treatment thereof.

One aspect of the present application provides a multispecific antibody (“multispecific T cell engager”) comprising: a first antigen-binding fragment that specifically binds CD3, wherein the first antigen-binding fragment is fused to a first masking moiety (MM1); and a second antigen-binding fragment that specifically binds a target antigen; wherein the MM1 competes with CD3 to specifically bind the CD3-binding moiety; and wherein the first antigen-binding fragment binds CD3 with half-maximal binding at a concentration of antibody (EC50) that is at least 10 nM as determined by an enzyme-linked immunosorbent assay (ELISA). In some embodiments, the EC₅₀ is at least 50 nM. In some embodiments, the EC₅₀ is at least 100 nM (e.g., about 110 nM). In some embodiments, the first antigen-binding fragment is a scFv, such as an isolated anti-CD3 scFv, an isolated anti-CD3 scFv-Fc fusion protein, or an anti-CD3 scFv fragment in a multispecific (e.g., bispecific) antibody without the MM1, when used to determine the EC₅₀. In some embodiments, the EC₅₀ is determined using the ELISA assay as described in Example 5.

In some embodiments according to any one of the multispecific antibodies described above, the multispecific antibody is a not an activatable multispecific antibody. In some embodiments, the first antigen-binding fragment comprises a first immunoglobulin light chain variable domain (VL1) and a first immunoglobulin heavy chain variable domain (VH1) of an anti-CD3 antibody, and wherein the MM1 is fused to the N-terminus of the VL1 via a first non-cleavable linker (NCL1).

One aspect of the present application provides an activatable multispecific antibody (“activatable multispecific T cell engager”) comprising: a) a first antigen-binding fragment that specifically binds CD3, wherein the first antigen-binding fragment is fused to a first masking moiety (MM1) via a first cleavable moiety (CM1); and b) a second antigen-binding fragment that specifically binds a target antigen; wherein the CM1 comprises a first cleavage site; wherein the MM1 competes with CD3 to specifically bind the CD3-binding moiety; and wherein the first antigen-binding fragment binds CD3 with half-maximal binding at a concentration of antibody (EC₅₀) that is at least 10 nM as determined by an enzyme-linked immunosorbent assay (ELISA). In some embodiments, the MM1 inhibits binding of the activatable antibody to CD3 when the CM1 is not cleaved; and the activatable multispecific antibody binds to CD3 via the first antigen-binding fragment when the CM1 is cleaved. In some embodiments, the first antigen-binding fragment is fused to the MM1 via a first cleavable moiety (CM1), the CM1 comprises a first cleavage site, the MM1 inhibits binding of the multispecific antibody to CD3 when the CM1 is not cleaved, and the multispecific antibody binds CD3 via the first antigen-binding fragment with higher affinity when the CM1 is cleaved, e.g., as compared to affinity of multispecific antibody binding to CD3 via the first antigen-binding fragment when the CM1 is not cleaved. In some embodiments, the EC₅₀ is at least 50 nM. In some embodiments, the EC₅₀ is at least 100 nM (e.g., about 110 nM). In some embodiments, the first antigen-binding fragment is a scFv, such as an isolated anti-CD3 scFv, an isolated anti-CD3 scFv-Fc fusion protein, or an anti-CD3 scFv fragment in a multispecific (e.g., bispecific) antibody or an activatable multispecific antibody in an activated form (i.e., with CM1 cleaved or effective binding by highly localized antigen concentration in the TME vs normal tissues), when used to determine the EC₅₀. In some embodiments, the EC₅₀ is determined using the ELISA assay as described in Example 5.

In some embodiments according to any one of the multispecific or activatable multispecific antibodies described above, the first antigen-binding fragment binds CD3 with a dissociation constant (Kd) of at least 50 nM or at least 100 nM. In some embodiments, the first antigen-binding fragment is a scFv, such as an isolated anti-CD3 scFv, an isolated anti-CD3 scFv-Fc fusion protein, or an anti-CD3 scFv fragment in a multispecific (e.g., bispecific) antibody or an activatable multispecific antibody in an activated form (e.g., with CM1 of the multispecific antibody cleaved or effective binding by highly localized antigen concentration in the TME vs normal tissues), when used to determine the Kd. In some embodiments, binding of an antigen-binding fragment to CD3 is measured when the antigen-binding fragment is unmasked.

In some embodiments according to any one of the multispecific or activatable multispecific antibodies described above, the MM1 has a masking efficiency of at least 250 (e.g., at least 500, 1000, 2000, 3000, 5000, 10000 or higher) as determined by an ELISA assay, e.g., the ELISA assay in Example 3. In some embodiments, the MM1 has a masking efficiency of at least 50 (e.g., at least 100, 200, 300, 400, 500, 600, 800, 1000 or higher) as determined by a Jurkat NFAT reporter assay, e.g., the Jurkat NFAT assay for the antigen concentration used in Example 3.

In some embodiments according to any one of the multispecific or activatable multispecific antibodies described above, the first antigen-binding fragment comprises a first immunoglobulin light chain variable domain (VL1) and a first immunoglobulin heavy chain variable domain (VH1) of an anti-CD3 antibody. In some embodiments, the first antigen-binding fragment is selected from the group consisting of a Fab, a Fv, a scFab and a scFv. In some embodiments, the first antigen-binding fragment is a scFv. In some embodiments, the scFv comprises from N-terminus to C-terminus, VL1, a linker, and VH1. In some embodiments, the scFv comprises from N-terminus to C-terminus, VH1, a linker, and VL1. In some embodiments in which the antibody is an activatable multispecific antibody, the MM1 is fused to the N-terminus of the VL1 via the CM1. In some embodiments in which the antibody is not an activatable multispecific antibody, the MM1 is fused to the N-terminus of the VL1 via the NCL1. In some embodiments, the multispecific antibody is not an activatable multispecific antibody. In some embodiments, the multispecific antibody does not comprise a cleavable linker. In some embodiments, the masking moiety is not fused with a sequence comprising a cleavage site.

In some embodiments according to any one of the multispecific or activatable multispecific antibodies described above, the second antigen-binding fragment comprises a second immunoglobulin light chain variable domain (VL2) and a second immunoglobulin heavy chain variable domain (VH2) of an antibody that specifically binds the target antigen. In some embodiments, the second antigen-binding fragment is selected from the group consisting of a Fab, a Fv, a scFab and a scFv. In some embodiments, the second antigen-binding fragment is a Fv. In some embodiments, the second antigen-binding fragment is a Fab. In some embodiments in which the antibody is not an activatable multispecific antibody, the multispecific antibody comprises a first polypeptide, a second polypeptide, and a third polypeptide, wherein:
(i) the first polypeptide comprises a structure represented by the formula:

VH2-CH1-hinge-CH2-first CH3 (1a);

(ii) the second polypeptide comprises a structure represented by the formula:

MM1-NCL1-VL1-VH1-hinge-CH2-second CH3 (1b);

and
(iii) the third polypeptide comprises a structure represented by the formula:

VL2-CL (1c);

wherein:
   CL is an immunoglobulin light chain constant domain;
   CH1 is an immunoglobulin heavy chain constant domain 1;
   CH2 is an immunoglobulin heavy chain constant domain 2;
   first CH3 is a first immunoglobulin heavy chain constant domain 3;
   second CH3 is a second immunoglobulin heavy chain constant domain 3;
   hinge is an immunoglobulin hinge region connecting the CH1 and CH2 domains;
      wherein the VL1 and the VH1 associate to form a scFv that specifically binds CD3; and wherein the VL2 and the VH2 associate to form a Fv that specifically binds the target antigen. In some embodiments in which the antibody is an activatable multispecific antibody, the activatable multispecific antibody comprises a first polypeptide, a second polypeptide, and a third polypeptide, wherein:
(i) the first polypeptide comprises a structure represented by the formula:

VH2-CH1-hinge-CH2-first CH3 (1a);

(ii) the second polypeptide comprises a structure represented by the formula:

MM1-CM1-VL1-VH1-hinge-CH2-second CH3 (1b);

and
(iii) the third polypeptide comprises a structure represented by the formula:

VL2-CL (1c);

wherein:
   CL is an immunoglobulin light chain constant domain;
   CH1 is an immunoglobulin heavy chain constant domain 1;
   CH2 is an immunoglobulin heavy chain constant domain 2;
   first CH3 is a first immunoglobulin heavy chain constant domain 3;
   second CH3 is a second immunoglobulin heavy chain constant domain 3;
   hinge is an immunoglobulin hinge region connecting the CH1 and CH2 domains;
      wherein the VL1 and the VH1 associate to form a scFv that specifically binds CD3; and wherein the VL2 and the VH2 associate to form a Fv that specifically binds the target antigen. In some embodiments, the multispecific antibody (e.g., the second polypeptide thereof) comprising an amino acid linker between VL1 and VH1.

In some embodiments according to any one of the multispecific or activatable multispecific antibodies described above, the second antigen-binding fragment is fused to a second masking moiety (MM2), wherein the MM2 competes with the target antigen to specifically bind the second antigen-binding fragment. In some embodiments, the second antigen-binding fragment is fused to the MM2 via a second non-cleavable linker (NCL2). In some embodiments, the second antigen-binding fragment is fused to the MM2 via a second cleavable moiety (CM2), wherein the CM2 comprises a second cleavage site, wherein the MM2 inhibits binding of the multispecific antibody to the target antigen when the CM2 is not cleaved, and wherein the multispecific antibody binds the target antigen via the second antigen-binding fragment when the CM2 is cleaved. In some embodiments, wherein the second antigen-binding fragment comprises a VH2 and a VL2 of an antibody that specifically binds the target antigen, the MM2 is fused to the N-terminus of the VL2 via the CM2. In some embodiments, the multispecific or activatable multispecific antibody comprises a first polypeptide, a second polypeptide, and a third polypeptide, wherein:
(i) the first polypeptide comprises a structure represented by the formula:

VH2-CH1-hinge-CH2-first CH3 (2a);

(ii) the second polypeptide comprises a structure represented by the formula:

MM1-CM1-VL1-VH1-hinge-CH2-second CH3 (2b);

and
(iii) the third polypeptide comprises a structure represented by the formula:

MM2-CM2-VL2-CL (2c);

wherein:
   CL is an immunoglobulin light chain constant domain;
   CH1 is an immunoglobulin heavy chain constant domain 1;
   CH2 is an immunoglobulin heavy chain constant domain 2;
   first CH3 is a first immunoglobulin heavy chain constant domain 3;
   second CH3 is a second immunoglobulin heavy chain constant domain 3;
   hinge is an immunoglobulin hinge region connecting the CH1 and CH2 domains;
      wherein the VL1 and the VH1 associate to form a scFv that specifically binds CD3; and wherein the VL2 and the VH2 associate to form a Fv that specifically binds the target antigen. In some embodiments, the multispecific antibody (e.g., the second polypeptide thereof) comprises an amino acid linker between VL1 and VH1.

In some embodiments according to any one of the multispecific or activatable multispecific antibodies described above, the CD3 is human CD3. In some embodiments, the first antigen-binding fragment is cross-reactive with a CD3 polypeptide from at least one non-human species selected from the group consisting of cynomolgus monkey, mouse, rat and dog.

In some embodiments according to any one of the multispecific or activatable multispecific antibodies described above, wherein the first antigen-binding fragment comprises a VH1 and a VL1 of an anti-CD3 antibody, the VH1 comprises a heavy chain complementarity determining region (CDR-H) 1 comprising the amino acid sequence according to Formula (I): X₁YAX₂X₃(SEQ ID NO: 382), wherein X₁ is D, S, or T, X₂ is I, L, or M, and X₃ is N or T, a CDR-H2 comprising the amino acid sequence according to Formula (II): RIRSKYNNYATYYAX₁X₂VKX₃ (SEQ ID NO: 383), wherein X₁ is D or E, X₂ is S or T, and X₃ is D, G, or S, and a CDR-H3 comprising the amino acid sequence according to Formula (III): HGNX₁GX₂SYVSX₃X₄AY (SEQ ID NO: 384), wherein X₁ is F or Y, X₂ is N or T, X₃ is W or Y, and X₄ is F or W. In some embodiments, the VL1 comprises a CDR-L1 comprising the amino acid sequence according to Formula (IV): X₁SSTGAVTX₂X₃NYX₄N (SEQ ID NO: 385), wherein X₁ is A, G, or R, X₂ is S or T, X₃ is G or S, and X₄ is A, P, or V, a CDR-L2 comprising the amino acid sequence according to Formula (V): GTX₁X₂RAP (SEQ ID NO: 386), wherein X₁ is K or N, and X₂ is F or K, and a CDR-L3 comprising the amino acid sequence according to Formula (VI): ALWYSX₁X₂WV (SEQ ID NO: 387), wherein X₁ is D, N, or T, and X₂ is L or R.

In some embodiments according to any one of the multispecific or activatable multispecific antibodies described above, wherein the first antigen-binding fragment comprises a VH1 and a VL1 of an anti-CD3 antibody, the VH1 comprises a heavy chain complementarity determining region (CDR-H) 1 comprising an amino acid sequence selected from the group consisting of SEQ ID NOs: 376, 390, 601, and 602, or a variant thereof comprising up to about 3 amino acid substitutions, a CDR-H2 comprising an amino acid sequence selected from the group consisting of SEQ ID NOs: 377, 391-394, and 603, or a variant thereof comprising up to about 3 amino acid substitutions, and a CDR-H3 comprising an amino acid sequence selected from the group consisting of SEQ ID NOs: 378, 395, 604, and 605, or a variant thereof comprising up to about 3 amino acid substitutions; and the VL1 comprises a CDR-L1 comprising an amino acid sequence selected from the group consisting of SEQ ID NOs: 396-398, and 606-609, or a variant thereof comprising up to about 3 amino acid substitutions, a CDR-L2 comprising an amino acid sequence selected from the group consisting of SEQ ID NOs: 380 and 399, or a variant thereof comprising up to about 3 amino acid substitutions, and a CDR-L3 comprising an amino acid sequence selected from the group consisting of SEQ ID NOs: 381, 400-401, and 610, or a variant thereof comprising up to about 3 amino acid substitutions.

In some embodiments according to any one of the multispecific or activatable multispecific antibodies described above, wherein the first antigen-binding fragment comprises a VH1 and a VL1 of an anti-CD3 antibody, the VH1 comprises a CDR-H1 comprising an amino acid sequence selected from the group consisting of SEQ ID NOs: 376 and 390, or a variant thereof comprising up to about 3 amino acid substitutions, a CDR-H2 comprising an amino acid sequence selected from the group consisting of SEQ ID NOs: 391-394, or a variant thereof comprising up to about 3 amino acid substitutions, and a CDR-H3 comprising an amino acid sequence selected from the group consisting of SEQ ID NOs: 378 and 395, or a variant thereof comprising up to about 3 amino acid substitutions; and the VL1 comprises a CDR-L1 comprising an amino acid sequence selected from the group consisting of SEQ ID NOs: 396-398, or a variant thereof comprising up to about 3 amino acid substitutions, a CDR-L2 comprising an amino acid sequence selected from the group consisting of SEQ ID NOs: 380 and 399, or a variant thereof comprising up to about 3 amino acid substitutions, and a CDR-L3 comprising an amino acid sequence selected from the group consisting of SEQ ID NOs: 381, and 400-401, or a variant thereof comprising up to about 3 amino acid substitutions.

In some embodiments according to any one of the multispecific or activatable multispecific antibodies described above, wherein the first antigen-binding fragment comprises a VH1 and a VL1 of an anti-CD3 antibody, the VH1 comprises a heavy chain complementarity determining region (CDR-H) 1 comprising the amino acid sequence of SEQ ID NO: 382, a CDR-H2 comprising the amino acid sequence of SEQ ID NO: 383, and a CDR-H3 comprising the amino acid sequence of SEQ ID NO: 384; and the VL1 comprises a light chain complementarity determining region (CDR-L) 1 comprising the amino acid sequence of SEQ ID NO: 385, a CDR-L2 comprising the amino acid sequence of SEQ ID NO: 386, and a CDR-L3 comprising the amino acid sequence of SEQ ID NO: 387. In some embodiments, the VH1 comprises a CDR-H1 comprising an amino acid sequence selected from the group consisting of SEQ ID NOs: 376 and 390, a CDR-H2 comprising an amino acid sequence selected from the group consisting of SEQ ID NOs: 391-394, and a CDR-H3 comprising an amino acid sequence selected from the group consisting of SEQ ID NOs: 378 and 395; and the VL1 comprises a CDR-L1 comprising an amino acid sequence selected from the group consisting of SEQ ID NOs: 396-398, a CDR-L2 comprising an amino acid sequence selected from the group consisting of SEQ ID NOs: 380 and 399, and a CDR-L3 comprising an amino acid sequence selected from the group consisting of SEQ ID NOs: 381, and 400-401. In some embodiments, the VH1 comprises a CDR-H1 comprising the amino acid sequence of SEQ ID NO: 376, a CDR-H2 comprising the amino acid sequence of SEQ ID NO: 391, and a CDR-H3 comprising the amino acid sequence of SEQ ID NO: 378; and the VL1 comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH1 comprises a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and the VL1 comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH1 comprises a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and the VL1 comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 401. In some embodiments, the VH1 comprises a CDR-H1 comprising the amino acid sequence of SEQ ID NO: 390, a CDR-H2 comprising the amino acid sequence of SEQ ID NO: 393, and a CDR-H3 comprising the amino acid sequence of SEQ ID NO: 395; and the VL1 comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH1 comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL1 comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 401. In some embodiments, the VH1 comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL1 comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH1 comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL1 comprises a CDR-L1 comprising the amino acid sequence of SEQ ID NO: 398, a CDR-L2 comprising the amino acid sequence of SEQ ID NO: 399, and a CDR-L3 comprising the amino acid sequence of SEQ ID NO: 400. In some embodiments, the VH1 comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL1 comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH1 comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 395; and the VL1 comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH1 comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL1 comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH1 comprises a CDR-H1 comprising the amino acid sequence of SEQ ID NO: 376, a CDR-H2 comprising the amino acid sequence of SEQ ID NO: 391, and a CDR-H3 comprising the amino acid sequence of SEQ ID NO: 378; and the VL1 comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH1 comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL1 comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH1 comprises a CDR-H1 comprising the amino acid sequence of SEQ ID NO: 390, a CDR-H2 comprising the amino acid sequence of SEQ ID NO: 393, and a CDR-H3 comprising the amino acid sequence of SEQ ID NO: 378; and the VL1 comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH1 comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 378; and the VL1 comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH1 comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 378; and the VL1 comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH1 comprises the amino acid sequence according to Formula (VII): EVQLVESGGGLVXIPGGSLRLSCAASGFTFX₂X₃YAIX₄WVRQAPGKGLEWVX₅RIRSKY NNYATYYAX₆SVKX₇RFTISRDX₈SKNTLYLQX₉NSLRAEDTAVYYCX₁₀RHGNX₁₁GX₁₂S YVSWFAYWGQGTLVTVSS (SEQ ID NO: 388), wherein X₁ is K or Q, X₂ is N or S, X₃ is S or T, X₄ is H or N, X₅ is G or S, X₆ is D or E, X₇ is D or G, X₈ is D or N, X₉ is I or L, X₁₀ is A or V, X₁₁ is F or Y, X₁₂ is N or T; and the VL1 comprises the amino acid sequence according to Formula (VIII): X₁AVVTQEPSLTVSPGGTVTLTCX₂SSTGAVTTSNYX₃NWX₄QQKPGQAPRGLIGGTX₅X₆ RAPGX₇PARFSGSLLGGKAALTLSGAQPEDEAEYYCALWYSX₈X₉WVFGGGTKLTVL (SEQ ID NO: 389), wherein X₁ is E or Q, X₂ is A, G, P, or R, X₃ is A or P, X₄ is F or V, X₅ is K or N, X₆ is F or K, X₇ is A, I, T, or V, X₈ is A, D, N, or T, and X₉ is H or L. In some embodiments, the VH1 comprises an amino acid sequence selected from the group consisting of SEQ ID NOs: 67, 402, 405, 407, 409, 410, 412, 414-416, and 611-640, or a variant thereof having at least about 80% sequence identity to an amino acid sequence selected from the group consisting of SEQ ID NOs: 67, 402, 405, 407, 409, 410, 412, 414-416, and 611-640; and the VL1 comprises an amino acid sequence selected from the group consisting of SEQ ID NOs: 68, 403, 404, 406, 408, 411, 413, and 641-666, or a variant thereof having at least about 80% sequence identity to an amino acid sequence selected from the group consisting of SEQ ID NOs: 68, 403, 404, 406, 408, 411, 413, and 641-666. In some embodiments, the VH1 comprises an amino acid sequence selected from the group consisting of SEQ ID NOs: 67, 402, 405, 407, 409, 410, 412, 414, 415, and 416; and the VL1 comprises an amino acid sequence selected from the group consisting of SEQ ID NOs: 68, 403, 404, 406, 408, 411, and 413. In some embodiments, the VH1 comprises the amino acid sequence of SEQ ID NO: 388, and the VL1 comprises the amino acid sequence of SEQ ID NO: 389. In some embodiments, the VH1 comprises an amino acid sequence selected from the group consisting of SEQ ID NOs: 402, 405, 407, 409, 410, 412, 414, 415, and 416; and the VL1 comprises an amino acid sequence selected from the group consisting of SEQ ID NOs: 403, 404, 406, 408, 411, and 413. In some embodiments, the VH1 comprises the amino acid sequence of SEQ ID NO: 402, and the VL1 comprises the amino acid sequence of SEQ ID NO: 403. In some embodiments, the VH1 comprises the amino acid sequence of SEQ ID NO: 402, and the VL1 comprises the amino acid sequence of SEQ ID NO: 404. In some embodiments, the VH1 comprises the amino acid sequence of SEQ ID NO: 405, and the VL1 comprises the amino acid sequence of SEQ ID NO: 406. In some embodiments, the VH1 comprises the amino acid sequence of SEQ ID NO: 407, and the VL1 comprises the amino acid sequence of SEQ ID NO: 404. In some embodiments, the VH1 comprises the amino acid sequence of SEQ ID NO: 407, and the VL1 comprises the amino acid sequence of SEQ ID NO: 403. In some embodiments, the VH1 comprises the amino acid sequence of SEQ ID NO: 407, and the VL1 comprises the amino acid sequence of SEQ ID NO: 408. In some embodiments, the VH1 comprises the amino acid sequence of SEQ ID NO: 409, and the VL1 comprises the amino acid sequence of SEQ ID NO: 408. In some embodiments, the VH1 comprises the amino acid sequence of SEQ ID NO: 410, and the VL1 comprises the amino acid sequence of SEQ ID NO: 411. In some embodiments, the VH1 comprises the amino acid sequence of SEQ ID NO: 412, and the VL1 comprises the amino acid sequence of SEQ ID NO: 413. In some embodiments, the VH1 comprises the amino acid sequence of SEQ ID NO: 410, and the VL1 comprises the amino acid sequence of SEQ ID NO: 413. In some embodiments, the VH1 comprises the amino acid sequence of SEQ ID NO: 414, and the VL1 comprises the amino acid sequence of SEQ ID NO: 403. In some embodiments, the VH1 comprises the amino acid sequence of SEQ ID NO: 415, and the VL1 comprises the amino acid sequence of SEQ ID NO: 413. In some embodiments, the VH1 comprises the amino acid sequence of SEQ ID NO: 416, and the VL1 comprises the amino acid sequence of SEQ ID NO: 413. In some embodiments, the VH1 comprises the amino acid sequence of SEQ ID NO: 416, and the VL1 comprises the amino acid sequence of SEQ ID NO: 411. In some embodiments, the first antigen-binding fragment comprises the amino acid sequence of SEQ ID NO: 421. In some embodiments, the first antigen-binding fragment comprises the amino acid sequence of SEQ ID NO: 422.

In some embodiments according to any one of the multispecific or activatable multispecific antibodies described above, the MM1 comprises the amino acid sequence of EVGSY (SEQ ID NO: 667) at the N-terminus of the MM1. In some embodiments, the MM1 comprises an amino acid sequence according to Formula (IX): PYDDPDCPSHX₁SDCDX₂ (SEQ ID NO: 668), wherein X₁ is D or E, and X₂ is N or Q. In some embodiments, the MM1 comprises an amino acid sequence according to Formula (X). In some embodiments, the MM1 comprises the amino acid sequence of SEQ ID NO: 417. In some embodiments, the MM1 comprises the amino acid sequence of SEQ ID NO: 35. In some embodiments, the MM1 comprises an amino acid sequence selected from the group consisting of SEQ ID NOs: 597-599.

In some embodiments according to any one of the activatable multispecific antibodies described above, the CM1 comprises an amino acid sequence selected from the group consisting of SEQ ID NOs: 77, 127-129, 418, 420, 431 and 477-490, and 516-555. In some embodiments, the CM1 comprises the amino acid sequence of SEQ ID NO: 77 or 418.

In some embodiments according to any one of the multispecific or activatable multispecific antibodies described above, the target antigen is a tumor antigen. In some embodiments, the tumor antigen is selected from the group consisting of CD19, CD20, EpCAM, CEA, PSMA, CD33, EGFR, HER2, EphA2, MCSP, ADAM17, PSCA, 17-A1, NKG2D, TROP2, CD79B, Nectin-4, BCMA, CD22, CD38, EGFR, GD2, SLAMF7, CD30, EpCAM, MUC1, MUC16, CD123, CD37, FOLR1, MET, FLT3, GPC3, CEACAM5, CLDN18, CSF1, Integrin alpha 5, NCAM1, PTPRC, CD138, NaPi2b, MSLN, DLL3, GPRC5D, GPNMB, ICAM1, SSTR2, carcinoma associated antigen CTAA16, CA9, ENG, ACVRL1, CD80, CSPG4, EGFL7, FLT1, HAVCR1, HGF, HLA-DRB, IGF1R, TPBG, ERBB3, and STEAP2. In some embodiments, the tumor antigen is HER2. In some embodiments, the tumor antigen is CD20. In some embodiments, the tumor antigen is TROP2. In some embodiments, the tumor antigen is BCMA. In some embodiments, the tumor antigen is CD19.

In some embodiments according to any one of the multispecific or activatable multispecific antibodies described above, the target antigen is HER2. In some embodiments, wherein the second antigen-binding fragment comprises a VH2 and a VL2 of an anti-HER2 antibody, the VH2 comprises a CDR-H1 comprising the amino acid sequence of SEQ ID NO: 423, a CDR-H2 comprising the amino acid sequence of SEQ ID NO: 424, and a CDR-H3 comprising the amino acid sequence of SEQ ID NO: 71; and the VL2 comprises a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the VH2 comprises the amino acid sequence of SEQ ID NO: 75, and the VL2 comprises the amino acid sequence of SEQ ID NO: 76. In some embodiments, the second antigen-binding fragment is fused to a second masking moiety (MM2) via a second cleavable moiety (CM2), wherein a) the MM2 comprises an amino acid sequence according to Formula (XI): ESX1X₂CX₃X₄DPFX₅CQX₆ (SEQ ID NO: 670), wherein X₁ is D or E, X₂ is A, F, V, or Y, X₃ is D or E, X₄ is A or L, X₅ is D or E, and X₆ is A, F, or Y; b) the MM2 comprises an amino acid sequence according to Formula (XII): X₁X₂X₃X₄X₅X₆CX₇X₈DPYECX₉X₁₀ (SEQ ID NO: 671), wherein X₁ is A, H, or S, X₂ is A, D, or S, X₃ is A, T, or V, X₄ is P, S, or T, X₅ is D or E, X₆ is A or V, X₇ is D or E, X₈ is A or L, X₉ is Q, S, or T, and X₁₀ is A, H, or V; or c) the MM2 comprises an amino acid sequence according to Formula (XIII): YNSDDDCX₁SX₂YDPYTCYY (SEQ ID NO: 672), wherein X₁ is A, I, or V, and X₂ is H or R. In some embodiments, the second antigen-binding fragment is fused to a second masking moiety (MM2) via a second cleavable moiety (CM2), wherein the MM2 comprises an amino acid sequence selected from the group consisting of SEQ ID NOs: 36, 419, 432-476, and 491-515. In some embodiments, the MM2 comprises the amino acid sequence of SEQ ID NO: 419. In some embodiments, the CM2 comprises an amino acid sequence selected from the group consisting of SEQ ID NOs: 77, 127-129, 418, 420, 431 and 477-490, and 516-555. In some embodiments, the CM2 comprises the amino acid sequence of SEQ ID NO: 420. In some embodiments, the CM2 comprises the amino acid sequence of SEQ ID NO: 77. In some embodiments, the multispecific or activatable multispecific antibody comprises: a first polypeptide comprising an amino acid sequence having at least 80% (e.g., at least 85%, 90%, 95%, 98%, or 99%; or 100%) sequence identity with SEQ ID NO: 425, a second polypeptide comprising an amino acid sequence having at least 80% (e.g., at least 85%, 90%, 95%, 98%, or 99%; or 100%) sequence identity with SEQ ID NO: 426, and a third polypeptide comprising an amino acid sequence having at least at least 80% (e.g., at least 85%, 90%, 95%, 98%, or 99%; or 100%) sequence identity with SEQ ID NO: 112. In some embodiments, the multispecific or activatable multispecific antibody comprises: a first polypeptide comprising an amino acid sequence having at least at least 80% (e.g., at least 85%, 90%, 95%, 98%, or 99%; or 100%) sequence identity with SEQ ID NO: 427, a second polypeptide comprising an amino acid sequence having at least 80% (e.g., at least 85%, 90%, 95%, 98%, or 99%; or 100%) sequence identity with SEQ ID NO: 428, and a third polypeptide comprising an amino acid sequence having at least 80% (e.g., at least 85%, 90%, 95%, 98%, or 99%; or 100%) sequence identity with SEQ ID NO: 112. In some embodiments, the multispecific or activatable multispecific antibody comprises: a first polypeptide comprising an amino acid sequence having at least at least 80% (e.g., at least 85%, 90%, 95%, 98%, or 99%; or 100%) sequence identity with SEQ ID NO: 429, a second polypeptide comprising an amino acid sequence having at least at least 80% (e.g., at least 85%, 90%, 95%, 98%, or 99%; or 100%) sequence identity with SEQ ID NO: 430, and a third polypeptide comprising an amino acid sequence having at least at least 80% (e.g., at least 85%, 90%, 95%, 98%, or 99%; or 100%) sequence identity with SEQ ID NO: 115. In some embodiments, the multispecific or activatable multispecific antibody comprises: a first polypeptide comprising an amino acid sequence having at least at least 80% (e.g., at least 85%, 90%, 95%, 98%, or 99%; or 100%) sequence identity with SEQ ID NO: 83, a second polypeptide comprising an amino acid sequence having at least at least 80% (e.g., at least 85%, 90%, 95%, 98%, or 99%; or 100%) sequence identity with SEQ ID NO: 84, and a third polypeptide comprising an amino acid sequence having at least at least 80% (e.g., at least 85%, 90%, 95%, 98%, or 99%; or 100%) sequence identity with SEQ ID NO: 85. In some embodiments, the multispecific or activatable multispecific antibody comprises: a first polypeptide comprising an amino acid sequence having at least at least 80% (e.g., at least 85%, 90%, 95%, 98%, or 99%; or 100%) sequence identity with SEQ ID NO: 683, a second polypeptide comprising an amino acid sequence having at least at least 80% (e.g., at least 85%, 90%, 95%, 98%, or 99%; or 100%) sequence identity with SEQ ID NO: 684, and a third polypeptide comprising an amino acid sequence having at least at least 80% (e.g., at least 85%, 90%, 95%, 98%, or 99%; or 100%) sequence identity with SEQ ID NO: 685. In some embodiments, the multispecific or activatable multispecific antibody comprises: a first polypeptide comprising an amino acid sequence having at least 80% (e.g., at least 85%, 90%, 95%, 98%, or 99%; or 100%) sequence identity with SEQ ID NO: 425 optionally without the C-terminal lysine, a second polypeptide comprising an amino acid sequence having at least 80% (e.g., at least 85%, 90%, 95%, 98%, or 99%; or 100%) sequence identity with SEQ ID NO: 426 optionally without the C-terminal lysine, and a third polypeptide comprising an amino acid sequence having at least at least 80% (e.g., at least 85%, 90%, 95%, 98%, or 99%; or 100%) sequence identity with SEQ ID NO: 112. In some embodiments, the multispecific or activatable multispecific antibody comprises: a first polypeptide comprising an amino acid sequence having at least at least 80% (e.g., at least 85%, 90%, 95%, 98%, or 99%; or 100%) sequence identity with SEQ ID NO: 427 optionally without the C-terminal lysine, a second polypeptide comprising an amino acid sequence having at least 80% (e.g., at least 85%, 90%, 95%, 98%, or 99%; or 100%) sequence identity with SEQ ID NO: 428 optionally without the C-terminal lysine, and a third polypeptide comprising an amino acid sequence having at least 80% (e.g., at least 85%, 90%, 95%, 98%, or 99%; or 100%) sequence identity with SEQ ID NO: 112. In some embodiments, the multispecific or activatable multispecific antibody comprises: a first polypeptide comprising an amino acid sequence having at least at least 80% (e.g., at least 85%, 90%, 95%, 98%, or 99%; or 100%) sequence identity with SEQ ID NO: 429 optionally without the C-terminal lysine, a second polypeptide comprising an amino acid sequence having at least at least 80% (e.g., at least 85%, 90%, 95%, 98%, or 99%; or 100%) sequence identity with SEQ ID NO: 430 optionally without the C-terminal lysine, and a third polypeptide comprising an amino acid sequence having at least at least 80% (e.g., at least 85%, 90%, 95%, 98%, or 99%; or 100%) sequence identity with SEQ ID NO: 115. In some embodiments, the multispecific or activatable multispecific antibody comprises: a first polypeptide comprising an amino acid sequence having at least at least 80% (e.g., at least 85%, 90%, 95%, 98%, or 99%; or 100%) sequence identity with SEQ ID NO: 83, a second polypeptide comprising an amino acid sequence having at least at least 80% (e.g., at least 85%, 90%, 95%, 98%, or 99%; or 100%) sequence identity with SEQ ID NO: 84 optionally without the C-terminal lysine, and a third polypeptide comprising an amino acid sequence having at least at least 80% (e.g., at least 85%, 90%, 95%, 98%, or 99%; or 100%) sequence identity with SEQ ID NO: 85 optionally without the C-terminal lysine. In some embodiments, the multispecific or activatable multispecific antibody comprises: a first polypeptide comprising an amino acid sequence having at least at least 80% (e.g., at least 85%, 90%, 95%, 98%, or 99%; or 100%) sequence identity with SEQ ID NO: 683, a second polypeptide comprising an amino acid sequence having at least at least 80% (e.g., at least 85%, 90%, 95%, 98%, or 99%; or 100%) sequence identity with SEQ ID NO: 684 optionally without the C-terminal lysine, and a third polypeptide comprising an amino acid sequence having at least at least 80% (e.g., at least 85%, 90%, 95%, 98%, or 99%; or 100%) sequence identity with SEQ ID NO: 685 optionally without the C-terminal lysine. In some embodiments according to any one of the multispecific or activatable multispecific antibodies described above, the multispecific or activatable multispecific antibody comprises a mixture of heavy chain species, wherein some species comprise the C-terminal lysine, and some species lack the C-terminal lysine.

In some embodiments according to any one of the multispecific or activatable multispecific antibodies described above, the target antigen is CD20. In some embodiments, wherein the second antigen-binding fragment comprises a VH2 and a VL2 of an anti-CD20 antibody, the VH2 comprises a CDR-H1 comprising the amino acid sequence of SEQ ID NO: 556, a CDR-H2 comprising the amino acid sequence of SEQ ID NO: 557, and a CDR-H3 comprising the amino acid sequence of SEQ ID NO: 558; and the VL2 comprises a CDR-L1 comprising the amino acid sequence of SEQ ID NO: 559, a CDR-L2 comprising the amino acid sequence of SEQ ID NO: 560, and a CDR-L3 comprising the amino acid sequence of SEQ ID NO: 561. In some embodiments, wherein the second antigen-binding fragment comprises a VH2 and a VL2 of an anti-CD20 antibody, the VH2 comprises a CDR-H1 comprising the amino acid sequence of SEQ ID NO: 86, a CDR-H2 comprising the amino acid sequence of SEQ ID NO: 557, and a CDR-H3 comprising the amino acid sequence of SEQ ID NO: 558; and the VL2 comprises a CDR-L1 comprising the amino acid sequence of SEQ ID NO: 559, a CDR-L2 comprising the amino acid sequence of SEQ ID NO: 560, and a CDR-L3 comprising the amino acid sequence of SEQ ID NO: 561. In some embodiments, the VH2 comprises the amino acid sequence of SEQ ID NO: 562, and the VL2 comprises the amino acid sequence of SEQ ID NO: 563. In some embodiments, the multispecific or activatable multispecific antibody comprises: a first polypeptide comprising an amino acid sequence having at least 80% (e.g., at least 85%, 90%, 95%, 98%, or 99%; or 100%) sequence identity with SEQ ID NO: 564, a second polypeptide comprising an amino acid sequence having at least 80% (e.g., at least 85%, 90%, 95%, 98%, or 99%; or 100%) sequence identity with SEQ ID NO: 565, and a third polypeptide comprising an amino acid sequence having at least at least 80% (e.g., at least 85%, 90%, 95%, 98%, or 99%; or 100%) sequence identity with SEQ ID NO: 567. In some embodiments, the multispecific or activatable multispecific antibody comprises: a first polypeptide comprising an amino acid sequence having at least 80% (e.g., at least 85%, 90%, 95%, 98%, or 99%; or 100%) sequence identity with SEQ ID NO: 564, a second polypeptide comprising an amino acid sequence having at least 80% (e.g., at least 85%, 90%, 95%, 98%, or 99%; or 100%) sequence identity with SEQ ID NO: 565, and a third polypeptide comprising an amino acid sequence having at least at least 80% (e.g., at least 85%, 90%, 95%, 98%, or 99%; or 100%) sequence identity with SEQ ID NO: 569. In some embodiments, the multispecific or activatable multispecific antibody comprises: a first polypeptide comprising an amino acid sequence having at least 80% (e.g., at least 85%, 90%, 95%, 98%, or 99%; or 100%) sequence identity with SEQ ID NO: 564, a second polypeptide comprising an amino acid sequence having at least 80% (e.g., at least 85%, 90%, 95%, 98%, or 99%; or 100%) sequence identity with SEQ ID NO: 565 optionally without the C-terminal lysine, and a third polypeptide comprising an amino acid sequence having at least at least 80% (e.g., at least 85%, 90%, 95%, 98%, or 99%; or 100%) sequence identity with SEQ ID NO: 567 optionally without the C-terminal lysine. In some embodiments, the multispecific or activatable multispecific antibody comprises: a first polypeptide comprising an amino acid sequence having at least 80% (e.g., at least 85%, 90%, 95%, 98%, or 99%; or 100%) sequence identity with SEQ ID NO: 564, a second polypeptide comprising an amino acid sequence having at least 80% (e.g., at least 85%, 90%, 95%, 98%, or 99%; or 100%) sequence identity with SEQ ID NO: 565 optionally without the C-terminal lysine, and a third polypeptide comprising an amino acid sequence having at least at least 80% (e.g., at least 85%, 90%, 95%, 98%, or 99%; or 100%) sequence identity with SEQ ID NO: 569 optionally without the C-terminal lysine. In some embodiments according to any one of the multispecific or activatable multispecific antibodies described above, the multispecific or activatable multispecific antibody comprises a mixture of heavy chain species, wherein some species comprise the C-terminal lysine, and some species lack the C-terminal lysine.

In some embodiments according to any one of the multispecific or activatable multispecific antibodies described above, the multispecific or activatable multispecific antibody comprises an Fc region. In some embodiments, the Fc region is of the human IgG1 subclass. In some embodiments, the Fc region is of the human IgG2 subclass. In some embodiments, the Fc region is of the human IgG4 subclass. In some embodiments, the Fc region has enhanced ADCC and/or cross-linking efficiency. In some embodiments, the Fc region has reduced or no antibody-dependent cell cytotoxicity (ADCC) effect and/or reduced or no cross-linking effect. In some embodiments, the Fc region is of the human IgG1 subclass and has an N297A amino acid substitution.

In some embodiments according to any one of the multispecific or activatable multispecific antibodies described above, wherein the multispecific or activatable multispecific antibody comprises a first CH3 domain and a second CH3 domain, i) the first CH3 domain comprises a cysteine (C) residue at position 390 and the second CH3 domain comprises a cysteine residue at position 400, or the first CH3 domain comprises a cysteine residue at position 400 and the second CH3 domain comprises a cysteine residue at position 390; or ii) the first CH3 domain comprises a cysteine residue at position 392 and the second CH3 domain comprises a cysteine residue at position 397, or the first CH3 domain comprises a cysteine residue at position 397 and the second CH3 domain comprises a cysteine residue at position 392; or iii) the first CH3 domain comprises a cysteine residue at position 392 and the second CH3 domain comprises a cysteine residue at position 400, or the first CH3 domain comprises a cysteine residue at position 400 and the second CH3 domain comprises a cysteine residue at position 392; and wherein the amino acid residue numbering is based on EU numbering. In some embodiments, i) the first CH3 domain comprises N390C substitution and the second CH3 domain comprises S400C substitution, or the first CH3 domain comprises S400C substitution and the second CH3 domain comprises N390C substitution; or ii) the first CH3 domain comprises K392C substitution and the second CH3 domain comprises V397C substitution, or the first CH3 domain comprises V397C substitution and the second CH3 domain comprises K392C substitution; or iii) the first CH3 domain comprises K392C substitution and the second CH3 domain comprises S400C substitution, or the first CH3 domain comprises S400C substitution and the second CH3 domain comprises K392C substitution. In some embodiments, i) the first CH3 domain further comprises a positively charged residue at position 357 and the second CH3 domain further comprises a negatively charged residue at position 351, or the first CH3 domain further comprises a negatively charged residue at position 351 and the second CH3 domain further comprises a positively charged residue at position 357; or ii) the first CH3 domain further comprises a positively charged residue at position 411 and the second CH3 domain further comprises a negatively charged residue at position 370, or the first CH3 domain further comprises a negatively charged residue at position 370 and the second CH3 domain further comprises a positively charged residue at position 411; or iii) the first CH3 domain further comprises a positively charged residue at position 364 and the second CH3 domain further comprises a negatively charged residue at position 370, or the first CH3 domain further comprises a negatively charged residue at position 370 and the second CH3 domain further comprises a positively charged residue at position 364; or a combination of i) and ii), or a combination of i) and iii), and wherein the amino acid residue numbering is based on EU numbering. In some embodiments, the first CH3 domain further comprises a positively charged residue at position 356 and the second CH3 domain further comprises a negatively charged residue at position 439, or first CH3 domain further comprises a negatively charged residue at position 439 and the second CH3 domain further comprises a positively charged residue at position 356; and wherein the amino acid residue numbering is based on EU numbering. In some embodiments, i) the positively charged residue is a lysine (K) residue, and the negatively charged residue is an aspartic acid (D) residue; or ii) the positively charged residue is a lysine (K) residue, and the negatively charged residue is a glutamic acid (E) residue; or iii) the positively charged residue is an arginine (R) residue, and the negatively charged residue is an aspartic acid (D) residue; or iv) the positively charged residue is an arginine (R) residue, and the negatively charged residue is a glutamic acid (E) residue. In some embodiments, i) the first CH3 domain comprises E357K and T411K substitutions and the second CH3 domain comprises L351D and K370D substitutions, or the first CH3 domain comprises L351D and K370D substitutions and the second CH3 domain comprises E357K and T411K substitutions; or ii) the first CH3 domain comprises E357K and S364K substitutions and the second CH3 domain comprises L351D and K370D substitutions, or the first CH3 domain comprises L351D and K370D substitutions and the second CH3 domain comprises E357K and S364K substitutions; or iii) the first CH3 domain comprises D356K, E357K and S364K substitutions and the second CH3 domain comprises L351D, K370D and K439D substitutions, or the first CH3 domain comprises L351D, K370D and K439D substitutions and the second CH3 domain comprises D356K, E357K and S364K substitutions. In some embodiments, i) the first CH3 domain further comprises K392D and K409D substitutions and the second CH3 domain further comprises D356K, and D399K substitutions, or the first CH3 domain further comprises D356K and D399K substitutions and the second CH3 domain further comprises K392D and K409D substitutions; or ii) the first CH3 domain further comprises L368D and K370S substitutions and the second CH3 domain further comprises E357Q and S364K substitutions, or the first CH3 domain further comprises E357Q and S364K substitutions and the second CH3 domain further comprises L368D and K370S substitutions; or iii) the first CH3 domain further comprises L351K and T366K substitutions and the second CH3 domain further comprises L351D and L368E substitutions, or the first CH3 domain further comprises L351D and L368E substitutions and the second CH3 domain further comprises L351K and T366K substitutions; or (iv) the first CH3 domain further comprises P395K, P396K and V397K substitutions and the second CH3 domain comprises T394D, P395D and P396D substitutions, or the first CH3 domain further comprises T394D, P395D and P396D substitutions and the second CH3 domain further comprises P395K, P396K and V397K substitutions; or (v) the first CH3 domain further comprises F405E, Y407E and K409E substitutions and the second CH3 domain comprises F405K and Y407K substitutions, or the first CH3 domain further comprises F405K and Y407K substitutions and the second CH3 domain further comprises F405E, Y407E and K409E substitutions.

In some embodiments according to any one of the multispecific or activatable multispecific antibodies described above, wherein the multispecific or activatable multispecific antibody comprises a first CH3 domain and a second CH3 domain, the first CH3 domain comprises E357K, S364K and N390C substitutions and the second CH3 domain comprises L351D, K370D, and S400C substitutions, or the first CH3 domain comprises L351D, K370D, and S400C substitutions and the second CH3 domain comprises E357K, S364K and N390C substitutions. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions.

In some embodiments according to any one of the multispecific or activatable multispecific antibodies described above, the multispecific or activatable multispecific antibody is a bispecific antibody.

One aspect of the present application provides an isolated antibody or antigen-binding fragment thereof that specifically binds CD3 (“anti-CD3 antibody”), comprising: a VH comprising a heavy chain complementarity determining region (CDR-H) 1 comprising the amino acid sequence according to Formula (I): X₁YAX₂X₃(SEQ ID NO: 382), wherein X₁ is D, S, or T, X₂ is I, L, or M, and X₃ is N or T, a CDR-H2 comprising the amino acid sequence according to Formula (II): RIRSKYNNYATYYAX₁X₂VKX₃ (SEQ ID NO: 383), wherein X₁ is D or E, X₂ is S or T, and X₃ is D, G, or S, and a CDR-H3 comprising the amino acid sequence according to Formula (III): HGNX₁GX₂SYVSX₃X₄AY (SEQ ID NO: 384), wherein X₁ is F or Y, X₂ is N or T, X₃ is W or Y, and X₄ is F or W; and b) a VL comprising a CDR-L1 comprising the amino acid sequence according to Formula (IV): X₁SSTGAVTX₂X₃NYX₄N (SEQ ID NO: 385), wherein X₁ is A, G, or R, X₂ is S or T, X₃ is G or S, and X₄ is A, P, or V, a CDR-L2 comprising the amino acid sequence according to Formula (V): GTX₁X₂RAP (SEQ ID NO: 386), wherein X₁ is K or N, and X₂ is F or K, and a CDR-L3 comprising the amino acid sequence according to Formula (VI): ALWYSX₁X₂WV (SEQ ID NO: 387), wherein X₁ is D, N, or T, and X₂ is L or R. In some embodiments the VH comprises a heavy chain complementarity determining region (CDR-H) 1 comprising an amino acid sequence selected from the group consisting of SEQ ID NOs: 376, 390, 601, and 602, or a variant thereof comprising up to about 3 amino acid substitutions, a CDR-H2 comprising an amino acid sequence selected from the group consisting of SEQ ID NOs: 377, 391-394, and 603, or a variant thereof comprising up to about 3 amino acid substitutions, and a CDR-H3 comprising an amino acid sequence selected from the group consisting of SEQ ID NOs: 378, 395, 604, and 605, or a variant thereof comprising up to about 3 amino acid substitutions; and the VL comprises a CDR-L1 comprising an amino acid sequence selected from the group consisting of SEQ ID NOs: 396-398, and 606-609, or a variant thereof comprising up to about 3 amino acid substitutions, a CDR-L2 comprising an amino acid sequence selected from the group consisting of SEQ ID NOs: 380 and 399, or a variant thereof comprising up to about 3 amino acid substitutions, and a CDR-L3 comprising an amino acid sequence selected from the group consisting of SEQ ID NOs: 381, 400-401, and 610, or a variant thereof comprising up to about 3 amino acid substitutions. In some embodiments the VH comprises a CDR-H1 comprising an amino acid sequence selected from the group consisting of SEQ ID NOs: 376 and 390, or a variant thereof comprising up to about 3 amino acid substitutions, a CDR-H2 comprising an amino acid sequence selected from the group consisting of SEQ ID NOs: 391-394, or a variant thereof comprising up to about 3 amino acid substitutions, and a CDR-H3 comprising an amino acid sequence selected from the group consisting of SEQ ID NOs: 378 and 395, or a variant thereof comprising up to about 3 amino acid substitutions; and the VL comprises a CDR-L1 comprising an amino acid sequence selected from the group consisting of SEQ ID NOs: 396-398, or a variant thereof comprising up to about 3 amino acid substitutions, a CDR-L2 comprising an amino acid sequence selected from the group consisting of SEQ ID NOs: 380 and 399, or a variant thereof comprising up to about 3 amino acid substitutions, and a CDR-L3 comprising an amino acid sequence selected from the group consisting of SEQ ID NOs: 381, and 400-401, or a variant thereof comprising up to about 3 amino acid substitutions. In some embodiments, the VH comprises a CDR-H1 comprising the amino acid sequence of SEQ ID NO: 382, a CDR-H2 comprising the amino acid sequence of SEQ ID NO: 383, and a CDR-H3 comprising the amino acid sequence of SEQ ID NO: 384; and a VL comprising a CDR-L1 comprising the amino acid sequence of SEQ ID NO: 385, a CDR-L2 comprising the amino acid sequence of SEQ ID NO: 386, and a CDR-L3 comprising the amino acid sequence of SEQ ID NO: 387. In some embodiments, the VH comprises a CDR-H1 comprising an amino acid sequence selected from the group consisting of SEQ ID NOs: 376 and 390, a CDR-H2 comprising an amino acid sequence selected from the group consisting of SEQ ID NOs: 391-394, and a CDR-H3 comprising an amino acid sequence selected from the group consisting of SEQ ID NOs: 378 and 395; and the VL comprises a CDR-L1 comprising an amino acid sequence selected from the group consisting of SEQ ID NOs: 396-398, a CDR-L2 comprising an amino acid sequence selected from the group consisting of SEQ ID NOs: 380 and 399, and a CDR-L3 comprising an amino acid sequence selected from the group consisting of SEQ ID NOs: 381, and 400-401. In some embodiments, the VH comprises a CDR-H1 comprising the amino acid sequence of SEQ ID NO: 376, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH comprises a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 401. In some embodiments, the VH comprises a CDR-H1 comprising the amino acid sequence of SEQ ID NO: 390, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 401. In some embodiments, the VH comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 398, a CDR-L2 comprising the amino acid sequence of SEQ ID NO: 399, and a CDR-L3 comprising the amino acid sequence of SEQ ID NO: 400. In some embodiments, the VH comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 376, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 393, and a CDR-H3 comprising the amino acid sequence of SEQ ID NO: 378; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH comprises the amino acid sequence according to Formula (VII): EVQLVESGGGLVXIPGGSLRLSCAASGFTFX₂X₃YAIX₄WVRQAPGKGLEWVX₅RIRSKY NNYATYYAX₆SVKX₇RFTISRDX₈SKNTLYLQX₉NSLRAEDTAVYYCX₁₀RHGNX₁₁GX₁₂S YVSWFAYWGQGTLVTVSS (SEQ ID NO: 388), wherein X₁ is K or Q, X₂ is N or S, X₃ is S or T, X₄ is H or N, X₅ is G or S, X₆ is D or E, X₇ is D or G, X₈ is D or N, X₉ is I or L, X₁₀ is A or V, X₁₁ is F or Y, X₁₂ is N or T; and the VL comprises the amino acid sequence according to Formula (VIII): X₁AVVTQEPSLTVSPGGTVTLTCX₂SSTGAVTTSNYX₃NWX₄QQKPGQAPRGLIGGTX₅X₆ RAPGX₇PARFSGSLLGGKAALTLSGAQPEDEAEYYCALWYSX₈X₉WVFGGGTKLTVL (SEQ ID NO: 389), wherein X₁ is E or Q, X₂ is A, G, P, or R, X₃ is A or P, X₄ is F or V, X₅ is K or N, X₆ is F or K, X₇ is A, I, T, or V, X₈ is A, D, N, or T, and X₉ is H or L. In some embodiments, the VH comprises an amino acid sequence selected from the group consisting of SEQ ID NOs: 67, 402, 405, 407, 409, 410, 412, 414-416, and 611-640, or a variant thereof having at least about 80% sequence identity to an amino acid sequence selected from the group consisting of SEQ ID NOs: 67, 402, 405, 407, 409, 410, 412, 414-416, and 611-640; and the VL comprises an amino acid sequence selected from the group consisting of SEQ ID NOs: 68, 403, 404, 406, 408, 411, 413, and 641-666, or a variant thereof having at least about 80% sequence identity to an amino acid sequence selected from the group consisting of SEQ ID NOs: 68, 403, 404, 406, 408, 411, 413, and 641-666. In some embodiments, the VH comprises an amino acid sequence selected from the group consisting of SEQ ID NOs: 67, 402, 405, 407, 409, 410, 412, 414, 415, and 416; and the VL comprises an amino acid sequence selected from the group consisting of SEQ ID NOs: 68, 403, 404, 406, 408, 411, and 413. In some embodiments according to any one of the isolated anti-CD3 antibodies or antigen-binding fragments thereof, the VH comprises an amino acid sequence selected from the group consisting of SEQ ID NOs: 402, 405, 407, 409, 410, 412, 414, 415, and 416; and the VL comprises an amino acid sequence selected from the group consisting of SEQ ID NOs: 403, 404, 406, 408, 411, and 413. In some embodiments, the VH comprises the amino acid sequence of SEQ ID NO: 388, and the VL comprises the amino acid sequence of SEQ ID NO: 389. In some embodiments, the VH comprises the amino acid sequence of SEQ ID NO: 402, and the VL comprises the amino acid sequence of SEQ ID NO: 403. In some embodiments, the VH comprises the amino acid sequence of SEQ ID NO: 402, and the VL comprises the amino acid sequence of SEQ ID NO: 404. In some embodiments, the VH comprises the amino acid sequence of SEQ ID NO: 405, and the VL comprises the amino acid sequence of SEQ ID NO: 406. In some embodiments, the VH comprises the amino acid sequence of SEQ ID NO: 407, and the VL comprises the amino acid sequence of SEQ ID NO: 404. In some embodiments, the VH comprises the amino acid sequence of SEQ ID NO: 407, and the VL comprises the amino acid sequence of SEQ ID NO: 403. In some embodiments, the VH comprises the amino acid sequence of SEQ ID NO: 407, and the VL comprises the amino acid sequence of SEQ ID NO: 408. In some embodiments, the VH comprises the amino acid sequence of SEQ ID NO: 409, and the VL comprises the amino acid sequence of SEQ ID NO: 408. In some embodiments, the VH comprises the amino acid sequence of SEQ ID NO: 410, and the VL comprises the amino acid sequence of SEQ ID NO: 411. In some embodiments, the VH comprises the amino acid sequence of SEQ ID NO: 412, and the VL comprises the amino acid sequence of SEQ ID NO: 413. In some embodiments, the VH comprises the amino acid sequence of SEQ ID NO: 410, and the VL comprises the amino acid sequence of SEQ ID NO: 413. In some embodiments, the VH comprises the amino acid sequence of SEQ ID NO: 414, and the VL comprises the amino acid sequence of SEQ ID NO: 403. In some embodiments, the VH comprises the amino acid sequence of SEQ ID NO: 415, and the VL comprises the amino acid sequence of SEQ ID NO: 413. In some embodiments, the VH comprises the amino acid sequence of SEQ ID NO: 416, and the VL comprises the amino acid sequence of SEQ ID NO: 413. In some embodiments, the VH comprises the amino acid sequence of SEQ ID NO: 416, and the VL comprises the amino acid sequence of SEQ ID NO: 411.

In some embodiments according to any one of the isolated anti-CD3 antibodies or antigen-binding fragments thereof, the anti-CD3 antibody further comprises a second antigen-binding fragment that specifically binds a target antigen. In some embodiments, the target antigen is a tumor antigen. In some embodiments, the tumor antigen is selected from the group consisting of CD19, CD20, EpCAM, CEA, PSMA, CD33, EGFR, HER2, EphA2, MCSP, ADAM17, PSCA, 17-A1, NKG2D, TROP2, CD79B, Nectin-4, BCMA, CD22, CD38, EGFR, GD2, SLAMF7, CD30, EpCAM, MUC1, MUC16, CD123, CD37, FOLR1, MET, FLT3, GPC3, CEACAM5, CLDN18, CSF1, Integrin alpha 5, NCAM1, PTPRC, CD138, NaPi2b, MSLN, DLL3, GPRC5D, GPNMB, ICAM1, SSTR2, carcinoma associated antigen CTAA16, CA9, ENG, ACVRL1, CD80, CSPG4, EGFL7, FLT1, HAVCR1, HGF, HLA-DRB, IGF1R, TPBG, ERBB3, and STEAP2. In some embodiments, the tumor antigen is HER2. In some embodiments, the tumor antigen is CD20. In some embodiments, the tumor antigen is TROP2. In some embodiments, the tumor antigen is BCMA. In some embodiments, the tumor antigen is CD19.

One aspect of the present application provides an activatable antibody (“activatable anti-CD3 antibody”), comprising, from N-terminus to C-terminus, a masking moiety (MM), a cleavable moiety (CM), and a CD3-binding moiety, wherein: a) the CD3-binding moiety comprises a VL and the activatable antibody further comprises a second polypeptide comprising a VH; b) the CD3-binding moiety comprises a VH and the activatable antibody further comprises a second polypeptide comprising a VL; c) the CD3-binding moiety comprises from the N-terminus to the C-terminus, a VL and a VH; or d) the CD3-binding moiety comprise from the N-terminus to the C-terminus, a VH and a VL; wherein the CM comprises a cleavage site; wherein the MM inhibits binding of the activatable antibody to CD3 when the CM is not cleaved; wherein the activatable antibody binds CD3 via the VH and the VL when the CM is cleaved; and wherein the activatable antibody binds CD3 with half-maximal binding at a concentration of antibody (EC₅₀) that is at least 10 nM (e.g., at least 50 nM, or at least 100 nM, or about 110 nM) as determined by an enzyme-linked immunosorbent assay (ELISA). In some embodiments, the first antigen-binding fragment is a scFv, such as an isolated anti-CD3 scFv, an isolated anti-CD3 scFv-Fc fusion protein, or an anti-CD3 scFv fragment in a multispecific (e.g., bispecific) antibody or an activatable multispecific antibody in an activated form (i.e., with CM1 cleaved), when used to determine the EC₅₀. In some embodiments, the EC₅₀ is determined using the ELISA assay as described in Example 5.

In some embodiments according to any one of the activatable anti-CD3 antibodies described above, the first antigen-binding fragment binds CD3 with a dissociation constant (Kd) of at least 50 nM. In some embodiments, the first antigen-binding fragment is a scFv, such as an isolated anti-CD3 scFv, an isolated anti-CD3 scFv-Fc fusion protein, or an anti-CD3 scFv fragment in a multispecific (e.g., bispecific) antibody or an activatable multispecific antibody in an activated form (i.e., with CM1 cleaved), when used to determine the Kd.

In some embodiments according to any one of the activatable anti-CD3 antibodies described above, the MM comprises the amino acid sequence of EVGSY (SEQ ID NO: 667) at the N-terminus of the MM. In some embodiments, the MM comprises an amino acid sequence according to Formula (IX): PYDDPDCPSHX₁SDCDX₂ (SEQ ID NO: 668), wherein X₁ is D or E, and X₂ is N or Q. In some embodiments, the MM comprises an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y. In some embodiments, the MM comprises the amino acid sequence of SEQ ID NO: 35. In some embodiments, the MM comprises the amino acid sequence of SEQ ID NO: 417. In some embodiments, the MM comprises an amino acid sequence selected from the group consisting of SEQ ID NOs: 585-588 and 597-591. In some embodiments, the CD3 is human CD3.

One aspect of the present application provides an activatable antibody (“activatable anti-CD3 antibody”), comprising, from N-terminus to C-terminus, a masking moiety (MM), a cleavable moiety (CM), and a CD3-binding moiety, wherein: a) the CD3-binding moiety comprises a VL and the activatable antibody further comprises a second polypeptide comprising a VH; b) the CD3-binding moiety comprises a VH and the activatable antibody further comprises a second polypeptide comprising a VL; c) the CD3-binding moiety comprises from the N-terminus to the C-terminus, a VL and a VH; or d) the CD3-binding moiety comprise from the N-terminus to the C-terminus, a VH and a VL; wherein the CM comprises a cleavage site; wherein the MM inhibits binding of the activatable antibody to CD3 when the CM is not cleaved; wherein the activatable antibody binds CD3 via the VH and the VL when the CM is cleaved; and wherein a) the MM comprises the amino acid sequence of EVGSY (SEQ ID NO: 667) at the N-terminus of the MM; b) the MM comprises an amino acid sequence according to Formula (IX): PYDDPDCPSHX₁SDCDX₂ (SEQ ID NO: 668), wherein X₁ is D or E, and X₂ is N or Q; or c) the MM comprises an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y. In some embodiments, the MM comprises the amino acid sequence of SEQ ID NOs: 35, 417, 585-588, and 597-599. In some embodiments, the CD3 is human CD3.

In some embodiments according to any one of the activatable anti-CD3 antibodies described above, the activatable anti-CD3 antibody comprises an anti-CD3 antigen-binding fragment selected from the group consisting of a Fab, a Fv, a scFab and a scFv. In some embodiments, the anti-CD3 antigen-binding fragment is a scFv. In some embodiments, the scFv comprises from the N-terminus to the C-terminus, the VL, a linker and the VH.

In some embodiments according to any one of the activatable anti-CD3 antibodies described above, the VH comprising a heavy chain complementarity determining region (CDR-H) 1 comprising the amino acid sequence according to Formula (I): X₁YAX₂X₃(SEQ ID NO: 382), wherein X₁ is D, S, or T, X₂ is I, L, or M, and X₃ is N or T, a CDR-H2 comprising the amino acid sequence according to Formula (II): RIRSKYNNYATYYAX₁X₂VKX₃ (SEQ ID NO: 383), wherein X₁ is D or E, X₂ is S or T, and X₃ is D, G, or S, and a CDR-H3 comprising the amino acid sequence according to Formula (III): HGNX₁GX₂SYVSX₃X₄AY (SEQ ID NO: 384), wherein X₁ is F or Y, X₂ is N or T, X₃ is W or Y, and X₄ is F or W; and b) the VL comprising a CDR-L1 comprising the amino acid sequence according to Formula (IV): X₁SSTGAVTX₂X₃NYX₄N (SEQ ID NO: 385), wherein X₁ is A, G, or R, X₂ is S or T, X₃ is G or S, and X₄ is A, P, or V, a CDR-L2 comprising the amino acid sequence according to Formula (V): GTX₁X₂RAP (SEQ ID NO: 386), wherein X₁ is K or N, and X₂ is F or K, and a CDR-L3 comprising the amino acid sequence according to Formula (VI): ALWYSX₁X₂WV (SEQ ID NO: 387), wherein X₁ is D, N, or T, and X₂ is L or R. In some embodiments the VH comprises a heavy chain complementarity determining region (CDR-H) 1 comprising an amino acid sequence selected from the group consisting of SEQ ID NOs: 376, 390, 601, and 602, or a variant thereof comprising up to about 3 amino acid substitutions, a CDR-H2 comprising an amino acid sequence selected from the group consisting of SEQ ID NOs: 377, 391-394, and 603, or a variant thereof comprising up to about 3 amino acid substitutions, and a CDR-H3 comprising an amino acid sequence selected from the group consisting of SEQ ID NOs: 378, 395, 604, and 605, or a variant thereof comprising up to about 3 amino acid substitutions; and the VL comprises a CDR-L1 comprising an amino acid sequence selected from the group consisting of SEQ ID NOs: 396-398, and 606-609, or a variant thereof comprising up to about 3 amino acid substitutions, a CDR-L2 comprising an amino acid sequence selected from the group consisting of SEQ ID NOs: 380 and 399, or a variant thereof comprising up to about 3 amino acid substitutions, and a CDR-L3 comprising an amino acid sequence selected from the group consisting of SEQ ID NOs: 381, 400-401, and 610, or a variant thereof comprising up to about 3 amino acid substitutions. In some embodiments the VH comprises a CDR-H1 comprising an amino acid sequence selected from the group consisting of SEQ ID NOs: 376 and 390, or a variant thereof comprising up to about 3 amino acid substitutions, a CDR-H2 comprising an amino acid sequence selected from the group consisting of SEQ ID NOs: 391-394, or a variant thereof comprising up to about 3 amino acid substitutions, and a CDR-H3 comprising an amino acid sequence selected from the group consisting of SEQ ID NOs: 378 and 395, or a variant thereof comprising up to about 3 amino acid substitutions; and the VL comprises a CDR-L1 comprising an amino acid sequence selected from the group consisting of SEQ ID NOs: 396-398, or a variant thereof comprising up to about 3 amino acid substitutions, a CDR-L2 comprising an amino acid sequence selected from the group consisting of SEQ ID NOs: 380 and 399, or a variant thereof comprising up to about 3 amino acid substitutions, and a CDR-L3 comprising an amino acid sequence selected from the group consisting of SEQ ID NOs: 381, and 400-401, or a variant thereof comprising up to about 3 amino acid substitutions. In some embodiments, the VH comprises a CDR-H1 comprising the amino acid sequence of SEQ ID NO: 382, a CDR-H2 comprising the amino acid sequence of SEQ ID NO: 383, and a CDR-H3 comprising the amino acid sequence of SEQ ID NO: 384; and the VL comprises a CDR-L1 comprising the amino acid sequence of SEQ ID NO: 385, a CDR-L2 comprising the amino acid sequence of SEQ ID NO: 386, and a CDR-L3 comprising the amino acid sequence of SEQ ID NO: 387. In some embodiments, the VH comprises a CDR-H1 comprising an amino acid sequence selected from the group consisting of SEQ ID NOs: 376 and 390, a CDR-H2 comprising an amino acid sequence selected from the group consisting of SEQ ID NOs: 391-394, and a CDR-H3 comprising an amino acid sequence selected from the group consisting of SEQ ID NOs: 378 and 395; and the VL comprises a CDR-L1 comprising an amino acid sequence selected from the group consisting of SEQ ID NOs: 396-398, a CDR-L2 comprising an amino acid sequence selected from the group consisting of SEQ ID NOs: 380 and 399, and a CDR-L3 comprising an amino acid sequence selected from the group consisting of SEQ ID NOs: 381, and 400-401. In some embodiments, the VH comprises a CDR-H1 comprising the amino acid sequence of SEQ ID NO: 376, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH comprises a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 401. In some embodiments, the VH comprises a CDR-H1 comprising the amino acid sequence of SEQ ID NO: 390, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 401. In some embodiments, the VH comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 398, a CDR-L2 comprising the amino acid sequence of SEQ ID NO: 399, and a CDR-L3 comprising the amino acid sequence of SEQ ID NO: 400. In some embodiments, the VH comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 376, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 393, and a CDR-H3 comprising the amino acid sequence of SEQ ID NO: 378; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH comprises the amino acid sequence of SEQ ID NO: 388, and the VL comprises the amino acid sequence of SEQ ID NO: 389.

In some embodiments according to any one of the activatable anti-CD3 antibodies described above, the VH comprises the amino acid sequence according to Formula (VII): EVQLVESGGGLVXIPGGSLRLSCAASGFTFX₂X₃YAIX₄WVRQAPGKGLEWVX₅RIRSKY NNYATYYAX₆SVKX₇RFTISRDX₈SKNTLYLQX₉NSLRAEDTAVYYCX₁₀RHGNX₁₁GX₁₂S YVSWFAYWGQGTLVTVSS (SEQ ID NO: 388), wherein X₁ is K or Q, X₂ is N or S, X₃ is S or T, X₄ is H or N, X₅ is G or S, X₆ is D or E, X₇ is D or G, X₈ is D or N, X₉ is I or L, X₁₀ is A or V, X₁₁ is F or Y, X₁₂ is N or T; and the VL comprises the amino acid sequence according to Formula (VIII): XIAVVTQEPSLTVSPGGTVTLTCX₂SSTGAVTTSNYX₃NWX₄QQKPGQAPRGLIGGTX₅X₆ RAPGX₇PARFSGSLLGGKAALTLSGAQPEDEAEYYCALWYSX₈X₉WVFGGGTKLTVL (SEQ ID NO: 389), wherein X₁ is E or Q, X₂ is A, G, P, or R, X₃ is A or P, X₄ is F or V, X₅ is K or N, X₆ is F or K, X₇ is A, I, T, or V, X₈ is A, D, N, or T, and X₉ is H or L. In some embodiments, the VH comprises an amino acid sequence selected from the group consisting of SEQ ID NOs: 67, 402, 405, 407, 409, 410, 412, 414-416, and 611-640, or a variant thereof having at least about 80% sequence identity to an amino acid sequence selected from the group consisting of SEQ ID NOs: 67, 402, 405, 407, 409, 410, 412, 414-416, and 611-640; and the VL comprises an amino acid sequence selected from the group consisting of SEQ ID NOs: 68, 403, 404, 406, 408, 411, 413, and 641-666, or a variant thereof having at least about 80% sequence identity to an amino acid sequence selected from the group consisting of SEQ ID NOs: 68, 403, 404, 406, 408, 411, 413, and 641-666. In some embodiments, the VH comprises an amino acid sequence selected from the group consisting of SEQ ID NOs: 67, 402, 405, 407, 409, 410, 412, 414, 415, and 416; and the VL comprises an amino acid sequence selected from the group consisting of SEQ ID NOs: 68, 403, 404, 406, 408, 411, and 413.

In some embodiments according to any one of the activatable anti-CD3 antibodies described above, the VH comprises an amino acid sequence selected from the group consisting of SEQ ID NOs: 402, 405, 407, 409, 410, 412, 414, 415, and 416; and the VL comprises an amino acid sequence selected from the group consisting of SEQ ID NOs: 403, 404, 406, 408, 411, and 413. In some embodiments, the VH comprises the amino acid sequence of SEQ ID NO: 402, and the VL comprises the amino acid sequence of SEQ ID NO: 403. In some embodiments, the VH comprises the amino acid sequence of SEQ ID NO: 402, and the VL comprises the amino acid sequence of SEQ ID NO: 404. In some embodiments, the VH comprises the amino acid sequence of SEQ ID NO: 405, and the VL comprises the amino acid sequence of SEQ ID NO: 406. In some embodiments, the VH comprises the amino acid sequence of SEQ ID NO: 407, and the VL comprises the amino acid sequence of SEQ ID NO: 404. In some embodiments, the VH comprises the amino acid sequence of SEQ ID NO: 407, and the VL comprises the amino acid sequence of SEQ ID NO: 403. In some embodiments, the VH comprises the amino acid sequence of SEQ ID NO: 407, and the VL comprises the amino acid sequence of SEQ ID NO: 408. In some embodiments, the VH comprises the amino acid sequence of SEQ ID NO: 409, and the VL comprises the amino acid sequence of SEQ ID NO: 408. In some embodiments, the VH comprises the amino acid sequence of SEQ ID NO: 410, and the VL comprises the amino acid sequence of SEQ ID NO: 411. In some embodiments, the VH comprises the amino acid sequence of SEQ ID NO: 412, and the VL comprises the amino acid sequence of SEQ ID NO: 413. In some embodiments, the VH comprises the amino acid sequence of SEQ ID NO: 410, and the VL comprises the amino acid sequence of SEQ ID NO: 413. In some embodiments, the VH comprises the amino acid sequence of SEQ ID NO: 414, and the VL comprises the amino acid sequence of SEQ ID NO: 403. In some embodiments, the VH comprises the amino acid sequence of SEQ ID NO: 415, and the VL comprises the amino acid sequence of SEQ ID NO: 413. In some embodiments, the VH comprises the amino acid sequence of SEQ ID NO: 416, and the VL comprises the amino acid sequence of SEQ ID NO: 413. In some embodiments, the VH comprises the amino acid sequence of SEQ ID NO: 416, and the VL comprises the amino acid sequence of SEQ ID NO: 411. In some embodiments, the CD3-binding moiety comprises the amino acid sequence of SEQ ID NO: 421 or SEQ ID NO: 422.

In some embodiments according to any one of the activatable anti-CD3 antibodies described above, the CM comprises an amino acid sequence selected from the group consisting of SEQ ID NOs: 77, 127-129, 418, 420, 431 and 477-490, and 516-555. In some embodiments, the CM comprises the amino acid sequence of SEQ ID NO: 77 or 418.

One aspect of the present application provides a masked antibody (“masked anti-CD3 antibody”), comprising, from N-terminus to C-terminus, a masking moiety (MM), and a CD3-binding moiety, wherein: a) the CD3-binding moiety comprises a VL and the activatable antibody further comprises a second polypeptide comprising a VH; b) the CD3-binding moiety comprises a VH and the activatable antibody further comprises a second polypeptide comprising a VL; c) the CD3-binding moiety comprises from the N-terminus to the C-terminus, a VL and a VH; or d) the CD3-binding moiety comprise from the N-terminus to the C-terminus, a VH and a VL; wherein the MM competes with CD3 to specifically bind the CD3-binding moiety; wherein the activatable antibody binds CD3 via the VH and the VL; and wherein the masked antibody binds CD3 with half-maximal binding at a concentration of antibody (EC₅₀) that is at least 10 nM (e.g., at least 50 nM, or at least 100 nM, or about 110 nM) as determined by an enzyme-linked immunosorbent assay (ELISA). In some embodiments, the masked anti-CD3 antibody is an activatable antibody. In some embodiments, the masked anti-CD3 antibody comprises, from N-terminus to C-terminus, the masking moiety (MM), a cleavable moiety (CM), and the CD3-binding moiety. In some embodiments, the masked anti-CD3 antibody is a not an activatable antibody. In some embodiments, the masked anti-CD3 antibody comprises, from N-terminus to C-terminus, the masking moiety (MM), a non-cleavable linker (NCL), and the CD3-binding moiety. One aspect of the present application provides a masked antibody (“masked anti-CD3 antibody”), comprising a masking moiety (MM) and an antibody or antigen-binding fragment that binds CD3, wherein the antibody or antigen-binding fragment comprises a VH and a VL; wherein the masked antibody comprises a single polypeptide chain and the VH and the VL of the antibody or antigen-binding fragment are part of the single polypeptide chain, or the masked antibody comprises two polypeptide chains, and the VH and the VL of the antibody or antigen-binding fragment are part of different polypeptide chains of the masked antibody; wherein the C-terminus of the MM is fused to the N-terminus of the VH or the VL of the antibody or antigen-binding fragment; wherein the MM competes with CD3 to specifically bind the antibody or antigen-binding fragment; and wherein the antibody or antigen-binding fragment binds CD3 with half-maximal binding at a concentration of antibody (EC₅₀) that is at least 10 nM (e.g., at least 50 nM, or at least 100 nM, or about 110 nM) as determined by an enzyme-linked immunosorbent assay (ELISA). In some embodiments, the masked antibody comprises an amino acid linker between the C-terminus of the MM and the N-terminus of the VH or the VL of the antibody or antigen-binding fragment. In some embodiments, the masked antibody further comprises a cleavable linker, e.g., between the C-terminus of the MM and the N-terminus of the VH or the VL of the antibody or antigen-binding fragment. In some embodiments, the masked antibody does not comprise a cleavable linker (e.g., fused to the MM, or between the C-terminus of the MM and the N-terminus of the antibody or fragment).

One aspect of the present application provides a masked antibody (“masked anti-CD3 antibody”), comprising, from N-terminus to C-terminus, a masking moiety (MM), a non-cleavable linker (NCL), and a CD3-binding moiety, wherein: a) the CD3-binding moiety comprises a VL and the activatable antibody further comprises a second polypeptide comprising a VH; b) the CD3-binding moiety comprises a VH and the activatable antibody further comprises a second polypeptide comprising a VL; c) the CD3-binding moiety comprises from the N-terminus to the C-terminus, a VL and a VH; or d) the CD3-binding moiety comprise from the N-terminus to the C-terminus, a VH and a VL; wherein the MM competes with CD3 to specifically bind the CD3-binding moiety; wherein the activatable antibody binds CD3 via the VH and the VL; and wherein the masked antibody binds CD3 with half-maximal binding at a concentration of antibody (EC₅₀) that is at least 10 nM (e.g., at least 50 nM, or at least 100 nM, or about 110 nM) as determined by an enzyme-linked immunosorbent assay (ELISA). In some embodiments, the first antigen-binding fragment is a scFv, such as an isolated anti-CD3 scFv, an isolated anti-CD3 scFv-Fc fusion protein, or an anti-CD3 scFv fragment in a multispecific (e.g., bispecific) antibody or multispecific antibody in an un-masked form (i.e., without the MM), when used to determine the EC₅₀. In some embodiments, the EC₅₀ is determined using the ELISA assay as described in Example 5.

In some embodiments according to any one of the masked anti-CD3 antibodies described above, the first antigen-binding fragment binds CD3 with a dissociation constant (Kd) of at least 50 nM. In some embodiments, the first antigen-binding fragment is a scFv, such as an isolated anti-CD3 scFv, an isolated anti-CD3 scFv-Fc fusion protein, or an anti-CD3 scFv fragment in a multispecific (e.g., bispecific) antibody or a multispecific antibody in an un-masked form (i.e., without the MM), when used to determine the Kd.

In some embodiments according to any one of the masked anti-CD3 antibodies described above, the MM comprises the amino acid sequence of EVGSY (SEQ ID NO: 667) at the N-terminus of the MM. In some embodiments, the MM comprises an amino acid sequence according to Formula (IX): PYDDPDCPSHX₁SDCDX₂ (SEQ ID NO: 668), wherein X₁ is D or E, and X₂ is N or Q. In some embodiments, the MM comprises an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y. In some embodiments, the MM comprises the amino acid sequence of SEQ ID NO: 35. In some embodiments, the MM comprises the amino acid sequence of SEQ ID NO: 417. In some embodiments, the MM comprises an amino acid sequence selected from the group consisting of SEQ ID NOs: 585-588 and 597-591. In some embodiments, the CD3 is human CD3.

One aspect of the present application provides a masked antibody (“masked anti-CD3 antibody”), comprising, from N-terminus to C-terminus, a masking moiety (MM), and a CD3-binding moiety, wherein: a) the CD3-binding moiety comprises a VL and the activatable antibody further comprises a second polypeptide comprising a VH; b) the CD3-binding moiety comprises a VH and the activatable antibody further comprises a second polypeptide comprising a VL; c) the CD3-binding moiety comprises from the N-terminus to the C-terminus, a VL and a VH; or d) the CD3-binding moiety comprise from the N-terminus to the C-terminus, a VH and a VL; wherein the MM competes with CD3 to specifically bind the CD3-binding moiety; wherein the activatable antibody binds CD3 via the VH and the VL; and wherein a) the MM comprises the amino acid sequence of EVGSY (SEQ ID NO: 667) at the N-terminus of the MM; b) the MM comprises an amino acid sequence according to Formula (IX): PYDDPDCPSHX₁SDCDX₂ (SEQ ID NO: 668), wherein X₁ is D or E, and X₂ is N or Q; or c) the MM comprises an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y. In some embodiments, the masked anti-CD3 antibody is an activatable antibody. In some embodiments, the masked anti-CD3 antibody comprises, from N-terminus to C-terminus, the masking moiety (MM), a cleavable moiety (CM), and the CD3-binding moiety. In some embodiments, the masked anti-CD3 antibody is a not an activatable antibody. In some embodiments, the masked anti-CD3 antibody comprises, from N-terminus to C-terminus, the masking moiety (MM), a non-cleavable linker (NCL), and the CD3-binding moiety.

One aspect of the present application provides a masked antibody (“masked anti-CD3 antibody”), comprising, from N-terminus to C-terminus, a masking moiety (MM), a non-cleavable linker (NCL), and a CD3-binding moiety, wherein: a) the CD3-binding moiety comprises a VL and the activatable antibody further comprises a second polypeptide comprising a VH; b) the CD3-binding moiety comprises a VH and the activatable antibody further comprises a second polypeptide comprising a VL; c) the CD3-binding moiety comprises from the N-terminus to the C-terminus, a VL and a VH; or d) the CD3-binding moiety comprise from the N-terminus to the C-terminus, a VH and a VL; wherein the MM competes with CD3 to specifically bind the CD3-binding moiety; wherein the activatable antibody binds CD3 via the VH and the VL; and wherein a) the MM comprises the amino acid sequence of EVGSY (SEQ ID NO: 667) at the N-terminus of the MM; b) the MM comprises an amino acid sequence according to Formula (IX): PYDDPDCPSHX₁SDCDX₂ (SEQ ID NO: 668), wherein X₁ is D or E, and X₂ is N or Q; or c) the MM comprises an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y. In some embodiments, the MM comprises the amino acid sequence of SEQ ID NOs: 35, 417, 585-588, and 597-599. In some embodiments, the CD3 is human CD3.

In some embodiments according to any one of the masked anti-CD3 antibodies described above, the activatable anti-CD3 antibody comprises an anti-CD3 antigen-binding fragment selected from the group consisting of a Fab, a Fv, a scFab and a scFv. In some embodiments, the anti-CD3 antigen-binding fragment is a scFv. In some embodiments, the scFv comprises from the N-terminus to the C-terminus, the VL, a linker and the VH.

In some embodiments according to any one of the masked anti-CD3 antibodies described above, the VH comprising a heavy chain complementarity determining region (CDR-H) 1 comprising the amino acid sequence according to Formula (I): X₁YAX₂X₃(SEQ ID NO: 382), wherein X₁ is D, S, or T, X₂ is I, L, or M, and X₃ is N or T, a CDR-H2 comprising the amino acid sequence according to Formula (II): RIRSKYNNYATYYAX₁X₂VKX₃ (SEQ ID NO: 383), wherein X₁ is D or E, X₂ is S or T, and X₃ is D, G, or S, and a CDR-H3 comprising the amino acid sequence according to Formula (III): HGNX₁GX₂SYVSX₃X₄AY (SEQ ID NO: 384), wherein X₁ is F or Y, X₂ is N or T, X₃ is W or Y, and X₄ is F or W; and b) the VL comprising a CDR-L1 comprising the amino acid sequence according to Formula (IV): X₁SSTGAVTX₂X₃NYX₄N (SEQ ID NO: 385), wherein X₁ is A, G, or R, X₂ is S or T, X₃ is G or S, and X₄ is A, P, or V, a CDR-L2 comprising the amino acid sequence according to Formula (V): GTX₁X₂RAP (SEQ ID NO: 386), wherein X₁ is K or N, and X₂ is F or K, and a CDR-L3 comprising the amino acid sequence according to Formula (VI): ALWYSX₁X₂WV (SEQ ID NO: 387), wherein X₁ is D, N, or T, and X₂ is L or R. In some embodiments the VH comprises a heavy chain complementarity determining region (CDR-H) 1 comprising an amino acid sequence selected from the group consisting of SEQ ID NOs: 376, 390, 601, and 602, or a variant thereof comprising up to about 3 amino acid substitutions, a CDR-H2 comprising an amino acid sequence selected from the group consisting of SEQ ID NOs: 377, 391-394, and 603, or a variant thereof comprising up to about 3 amino acid substitutions, and a CDR-H3 comprising an amino acid sequence selected from the group consisting of SEQ ID NOs: 378, 395, 604, and 605, or a variant thereof comprising up to about 3 amino acid substitutions; and the VL comprises a CDR-L1 comprising an amino acid sequence selected from the group consisting of SEQ ID NOs: 396-398, and 606-609, or a variant thereof comprising up to about 3 amino acid substitutions, a CDR-L2 comprising an amino acid sequence selected from the group consisting of SEQ ID NOs: 380 and 399, or a variant thereof comprising up to about 3 amino acid substitutions, and a CDR-L3 comprising an amino acid sequence selected from the group consisting of SEQ ID NOs: 381, 400-401, and 610, or a variant thereof comprising up to about 3 amino acid substitutions. In some embodiments the VH comprises a CDR-H1 comprising an amino acid sequence selected from the group consisting of SEQ ID NOs: 376 and 390, or a variant thereof comprising up to about 3 amino acid substitutions, a CDR-H2 comprising an amino acid sequence selected from the group consisting of SEQ ID NOs: 391-394, or a variant thereof comprising up to about 3 amino acid substitutions, and a CDR-H3 comprising an amino acid sequence selected from the group consisting of SEQ ID NOs: 378 and 395, or a variant thereof comprising up to about 3 amino acid substitutions; and the VL comprises a CDR-L1 comprising an amino acid sequence selected from the group consisting of SEQ ID NOs: 396-398, or a variant thereof comprising up to about 3 amino acid substitutions, a CDR-L2 comprising an amino acid sequence selected from the group consisting of SEQ ID NOs: 380 and 399, or a variant thereof comprising up to about 3 amino acid substitutions, and a CDR-L3 comprising an amino acid sequence selected from the group consisting of SEQ ID NOs: 381, and 400-401, or a variant thereof comprising up to about 3 amino acid substitutions. In some embodiments, the VH comprises a CDR-H1 comprising the amino acid sequence of SEQ ID NO: 382, a CDR-H2 comprising the amino acid sequence of SEQ ID NO: 383, and a CDR-H3 comprising the amino acid sequence of SEQ ID NO: 384; and the VL comprises a CDR-L1 comprising the amino acid sequence of SEQ ID NO: 385, a CDR-L2 comprising the amino acid sequence of SEQ ID NO: 386, and a CDR-L3 comprising the amino acid sequence of SEQ ID NO: 387. In some embodiments, the VH comprises a CDR-H1 comprising an amino acid sequence selected from the group consisting of SEQ ID NOs: 376 and 390, a CDR-H2 comprising an amino acid sequence selected from the group consisting of SEQ ID NOs: 391-394, and a CDR-H3 comprising an amino acid sequence selected from the group consisting of SEQ ID NOs: 378 and 395; and the VL comprises a CDR-L1 comprising an amino acid sequence selected from the group consisting of SEQ ID NOs: 396-398, a CDR-L2 comprising an amino acid sequence selected from the group consisting of SEQ ID NOs: 380 and 399, and a CDR-L3 comprising an amino acid sequence selected from the group consisting of SEQ ID NOs: 381, and 400-401. In some embodiments, the VH comprises a CDR-H1 comprising the amino acid sequence of SEQ ID NO: 376, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH comprises a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 401. In some embodiments, the VH comprises a CDR-H1 comprising the amino acid sequence of SEQ ID NO: 390, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 401. In some embodiments, the VH comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH comprises a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395; and the VL comprises a CDR-L1 comprising the amino acid sequence of SEQ ID NO: 398, a CDR-L2 comprising the amino acid sequence of SEQ ID NO: 399, and a CDR-L3 comprising the amino acid sequence of SEQ ID NO: 400. In some embodiments, the VH comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 376, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400. In some embodiments, the VH comprises a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 393, and a CDR-H3 comprising the amino acid sequence of SEQ ID NO: 378; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381. In some embodiments, the VH comprises a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 378; and the VL comprises a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the VH comprises the amino acid sequence of SEQ ID NO: 388, and the VL comprises the amino acid sequence of SEQ ID NO: 389.

In some embodiments according to any one of the masked anti-CD3 antibodies described above, the VH comprises the amino acid sequence according to Formula (VII): EVQLVESGGGLVXIPGGSLRLSCAASGFTFX₂X₃YAIX₄WVRQAPGKGLEWVX₅RIRSKY NNYATYYAX₆SVKX₇RFTISRDX₈SKNTLYLQX₉NSLRAEDTAVYYCX₁₀RHGNX₁₁GX₁₂S YVSWFAYWGQGTLVTVSS (SEQ ID NO: 388), wherein X₁ is K or Q, X₂ is N or S, X₃ is S or T, X₄ is H or N, X₅ is G or S, X₆ is D or E, X₇ is D or G, X₈ is D or N, X₉ is I or L, X₁₀ is A or V, X₁₁ is F or Y, X₁₂ is N or T; and the VL comprises the amino acid sequence according to Formula (VIII): XIAVVTQEPSLTVSPGGTVTLTCX₂SSTGAVTTSNYX₃NWX₄QQKPGQAPRGLIGGTX₅X₆ RAPGX₇PARFSGSLLGGKAALTLSGAQPEDEAEYYCALWYSX₈X₉WVFGGGTKLTVL (SEQ ID NO: 389), wherein X₁ is E or Q, X₂ is A, G, P, or R, X₃ is A or P, X₄ is F or V, X₅ is K or N, X₆ is F or K, X₇ is A, I, T, or V, X₈ is A, D, N, or T, and X₉ is H or L. In some embodiments, the VH comprises an amino acid sequence selected from the group consisting of SEQ ID NOs: 67, 402, 405, 407, 409, 410, 412, 414-416, and 611-640, or a variant thereof having at least about 80% sequence identity to an amino acid sequence selected from the group consisting of SEQ ID NOs: 67, 402, 405, 407, 409, 410, 412, 414-416, and 611-640; and the VL comprises an amino acid sequence selected from the group consisting of SEQ ID NOs: 68, 403, 404, 406, 408, 411, 413, and 641-666, or a variant thereof having at least about 80% sequence identity to an amino acid sequence selected from the group consisting of SEQ ID NOs: 68, 403, 404, 406, 408, 411, 413, and 641-666. In some embodiments, the VH comprises an amino acid sequence selected from the group consisting of SEQ ID NOs: 67, 402, 405, 407, 409, 410, 412, 414, 415, and 416; and the VL comprises an amino acid sequence selected from the group consisting of SEQ ID NOs: 68, 403, 404, 406, 408, 411, and 413.

In some embodiments according to any one of the masked anti-CD3 antibodies described above, the VH comprises an amino acid sequence selected from the group consisting of SEQ ID NOs: 402, 405, 407, 409, 410, 412, 414, 415, and 416; and the VL comprises an amino acid sequence selected from the group consisting of SEQ ID NOs: 403, 404, 406, 408, 411, and 413. In some embodiments, the VH comprises the amino acid sequence of SEQ ID NO: 402, and the VL comprises the amino acid sequence of SEQ ID NO: 403. In some embodiments, the VH comprises the amino acid sequence of SEQ ID NO: 402, and the VL comprises the amino acid sequence of SEQ ID NO: 404. In some embodiments, the VH comprises the amino acid sequence of SEQ ID NO: 405, and the VL comprises the amino acid sequence of SEQ ID NO: 406. In some embodiments, the VH comprises the amino acid sequence of SEQ ID NO: 407, and the VL comprises the amino acid sequence of SEQ ID NO: 404. In some embodiments, the VH comprises the amino acid sequence of SEQ ID NO: 407, and the VL comprises the amino acid sequence of SEQ ID NO: 403. In some embodiments, the VH comprises the amino acid sequence of SEQ ID NO: 407, and the VL comprises the amino acid sequence of SEQ ID NO: 408. In some embodiments, the VH comprises the amino acid sequence of SEQ ID NO: 409, and the VL comprises the amino acid sequence of SEQ ID NO: 408. In some embodiments, the VH comprises the amino acid sequence of SEQ ID NO: 410, and the VL comprises the amino acid sequence of SEQ ID NO: 411. In some embodiments, the VH comprises the amino acid sequence of SEQ ID NO: 412, and the VL comprises the amino acid sequence of SEQ ID NO: 413. In some embodiments, the VH comprises the amino acid sequence of SEQ ID NO: 410, and the VL comprises the amino acid sequence of SEQ ID NO: 413. In some embodiments, the VH comprises the amino acid sequence of SEQ ID NO: 414, and the VL comprises the amino acid sequence of SEQ ID NO: 403. In some embodiments, the VH comprises the amino acid sequence of SEQ ID NO: 415, and the VL comprises the amino acid sequence of SEQ ID NO: 413. In some embodiments, the VH comprises the amino acid sequence of SEQ ID NO: 416, and the VL comprises the amino acid sequence of SEQ ID NO: 413. In some embodiments, the VH comprises the amino acid sequence of SEQ ID NO: 416, and the VL comprises the amino acid sequence of SEQ ID NO: 411. In some embodiments, the CD3-binding moiety comprises the amino acid sequence of SEQ ID NO: 421 or SEQ ID NO: 422.

One aspect of the present application provides an activatable antibody (“activatable anti-HER2 antibody”) comprising, from N-terminus to C-terminus, a masking moiety (MM), a cleavable moiety (CM), and a HER2-binding moiety, wherein: a) the HER2-binding moiety comprises a VL and the activatable antibody further comprises a second polypeptide comprising a VH; b) the HER2-binding moiety comprises a VH and the activatable antibody further comprises a second polypeptide comprising a VL; c) the HER2-binding moiety comprises from the N-terminus to the C-terminus, a VL and a VH; or d) the HER2-binding moiety comprise from the N-terminus to the C-terminus, a VH and a VL; and wherein the CM comprises a cleavage site; wherein the MM inhibits binding of the activatable antibody to HER2 when the CM is not cleaved; and wherein the activatable antibody binds HER2 via the VH and VL when the CM is cleaved, and wherein the MM comprises: a) an amino acid sequence according to Formula (XI): ESX1X₂CX₃X₄DPFX₅CQX₆ (SEQ ID NO: 670), wherein X₁ is D or E, X₂ is A, F, V, or Y, X₃ is D or E, X₄ is A or L, X₅ is D or E, and X₆ is A, F, or Y; b) an amino acid sequence according to Formula (XII): X₁X₂X₃X₄X₅X₆CX₇X₈DPYECX₉X₁₀ (SEQ ID NO: 671), wherein X₁ is A, H, or S, X₂ is A, D, or S, X₃ is A, T, or V, X₄ is P, S, or T, X₅ is D or E, X₆ is A or V, X₇ is D or E, X₈ is A or L, X₉ is Q, S, or T, and X₁₀ is A, H, or V; or c) an amino acid sequence according to Formula (XIII): YNSDDDCX₁SX₂YDPYTCYY (SEQ ID NO: 672), wherein X₁ is A, I, or V, and X₂ is H or R. In some embodiments, the MM comprises an amino acid sequence selected from the group consisting of SEQ ID NOs: 36, 419, 432-476, and 491-515. In some embodiments, the CM comprises an amino acid sequence selected from the group consisting of SEQ ID NOs: 77, 127-129, 418, 420, 431 and 477-490, and 516-555.

In some embodiments according to any one of the activatable anti-HER2 antibodies described above, the VH comprises a CDR-H1 comprising the amino acid sequence of SEQ ID NO: 423, a CDR-H2 comprising the amino acid sequence of SEQ ID NO: 424, and a CDR-H3 comprising the amino acid sequence of SEQ ID NO: 71, and the VL comprises a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the VH comprises the amino acid sequence of SEQ ID NO: 75, and the VL comprises the amino acid sequence of SEQ ID NO: 76.

One aspect of the present application provides a masked antibody (“masked anti-HER2 antibody”) comprising, from N-terminus to C-terminus, a masking moiety (MM), and a HER2-binding moiety, wherein: a) the HER2-binding moiety comprises a VL and the activatable antibody further comprises a second polypeptide comprising a VH; b) the HER2-binding moiety comprises a VH and the activatable antibody further comprises a second polypeptide comprising a VL; c) the HER2-binding moiety comprises from the N-terminus to the C-terminus, a VL and a VH; or d) the HER2-binding moiety comprise from the N-terminus to the C-terminus, a VH and a VL; wherein the MM competes with HER2 to specifically bind the HER2-binding moiety; and wherein the activatable antibody binds HER2 via the VH and VL, and wherein the MM comprises: a) an amino acid sequence according to Formula (XI): ESX1X₂CX₃X₄DPFX₅CQX₆ (SEQ ID NO: 670), wherein X₁ is D or E, X₂ is A, F, V, or Y, X₃ is D or E, X₄ is A or L, X₅ is D or E, and X₆ is A, F, or Y; b) an amino acid sequence according to Formula (XII): X₁X₂X₃X₄X₅X₆CX₇X₈DPYECX₉X₁₀ (SEQ ID NO: 671), wherein X₁ is A, H, or S, X₂ is A, D, or S, X₃ is A, T, or V, X₄ is P, S, or T, X₅ is D or E, X₆ is A or V, X₇ is D or E, X₈ is A or L, X₉ is Q, S, or T, and X₁₀ is A, H, or V; or c) an amino acid sequence according to Formula (XIII): YNSDDDCX₁SX₂YDPYTCYY (SEQ ID NO: 672), wherein X₁ is A, I, or V, and X₂ is H or R.

In some embodiments, the masked anti-HER2 antibody is an activatable antibody. In some embodiments, the masked anti-HER2 antibody comprises, from N-terminus to C-terminus, the masking moiety (MM), a cleavable moiety (CM), and the HER2-binding moiety. In some embodiments, the masked anti-HER2 antibody is a not an activatable antibody. In some embodiments, the masked anti-HER2 antibody comprises, from N-terminus to C-terminus, the masking moiety (MM), a non-cleavable linker (NCL), and the HER2-binding moiety. One aspect of the present application provides a masked antibody (“masked anti-HER2 antibody”) comprising a masking moiety (MM) and an antibody or antigen-binding fragment that binds HER2, wherein the antibody or antigen-binding fragment comprises a VH and a VL; wherein the masked antibody comprises a single polypeptide chain and the VH and the VL of the antibody or antigen-binding fragment are part of the single polypeptide chain, or the masked antibody comprises two polypeptide chains, and the VH and the VL of the antibody or antigen-binding fragment are part of different polypeptide chains of the masked antibody; wherein the C-terminus of the MM is fused to the N-terminus of the VH or the VL of the antibody or antigen-binding fragment; wherein the MM competes with HER2 to specifically bind the antibody or antigen-binding fragment; and wherein the MM comprises: a) an amino acid sequence according to Formula (XI): ESX1X₂CX₃X₄DPFX₅CQX₆ (SEQ ID NO: 670), wherein X₁ is D or E, X₂ is A, F, V, or Y, X₃ is D or E, X₄ is A or L, X₅ is D or E, and X₆ is A, F, or Y; b) an amino acid sequence according to Formula (XII): X₁X₂X₃X₄X₅X₆CX₇X₈DPYECX₉X₁₀ (SEQ ID NO: 671), wherein X₁ is A, H, or S, X₂ is A, D, or S, X₃ is A, T, or V, X₄ is P, S, or T, X₅ is D or E, X₆ is A or V, X₇ is D or E, X₈ is A or L, X₉ is Q, S, or T, and X₁₀ is A, H, or V; or c) an amino acid sequence according to Formula (XIII): YNSDDDCX₁SX₂YDPYTCYY (SEQ ID NO: 672), wherein X₁ is A, I, or V, and X₂ is H or R. In some embodiments, the masked antibody comprises an amino acid linker between the C-terminus of the MM and the N-terminus of the VH or the VL of the antibody or antigen-binding fragment. In some embodiments, the masked antibody further comprises a cleavable linker, e.g., between the C-terminus of the MM and the N-terminus of the VH or the VL of the antibody or antigen-binding fragment. In some embodiments, the masked antibody does not comprise a cleavable linker (e.g., between the C-terminus of the MM and the N-terminus of the antibody or fragment).

One aspect of the present application provides a masked antibody (“masked anti-HER2 antibody”) comprising, from N-terminus to C-terminus, a masking moiety (MM), a non-cleavable linker (NCL), and a HER2-binding moiety, wherein: a) the HER2-binding moiety comprises a VL and the activatable antibody further comprises a second polypeptide comprising a VH; b) the HER2-binding moiety comprises a VH and the activatable antibody further comprises a second polypeptide comprising a VL; c) the HER2-binding moiety comprises from the N-terminus to the C-terminus, a VL and a VH; or d) the HER2-binding moiety comprise from the N-terminus to the C-terminus, a VH and a VL; wherein the MM competes with HER2 to specifically bind the HER2-binding moiety; and wherein the activatable antibody binds HER2 via the VH and VL, and wherein the MM comprises: a) an amino acid sequence according to Formula (XI): ESX1X₂CX₃X₄DPFX₅CQX₆ (SEQ ID NO: 670), wherein X₁ is D or E, X₂ is A, F, V, or Y, X₃ is D or E, X₄ is A or L, X₅ is D or E, and X₆ is A, F, or Y; b) an amino acid sequence according to Formula (XII): X₁X₂X₃X₄X₅X₆CX₇X₈DPYECX₉X₁₀ (SEQ ID NO: 671), wherein X₁ is A, H, or S, X₂ is A, D, or S, X₃ is A, T, or V, X₄ is P, S, or T, X₅ is D or E, X₆ is A or V, X₇ is D or E, X₈ is A or L, X₉ is Q, S, or T, and X₁₀ is A, H, or V; or c) an amino acid sequence according to Formula (XIII): YNSDDDCX₁SX₂YDPYTCYY (SEQ ID NO: 672), wherein X₁ is A, I, or V, and X₂ is H or R. In some embodiments, the MM comprises an amino acid sequence selected from the group consisting of SEQ ID NOs: 36, 419, 432-476, and 491-515.

In some embodiments according to any one of the masked anti-HER2 antibodies described above, the VH comprises a CDR-H1 comprising the amino acid sequence of SEQ ID NO: 423, a CDR-H2 comprising the amino acid sequence of SEQ ID NO: 424, and a CDR-H3 comprising the amino acid sequence of SEQ ID NO: 71, and the VL comprises a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the VH comprises the amino acid sequence of SEQ ID NO: 75, and the VL comprises the amino acid sequence of SEQ ID NO: 76.

In other aspects, the present disclosure provides an anti-HER2 antibody comprising the 6 CDRs and/or VH and VL sequences of any anti-HER2 binding domain provided herein. In some embodiments, an anti-HER2 antibody comprises a VH that comprises a CDR-H1 comprising the amino acid sequence of SEQ ID NO: 423, a CDR-H2 comprising the amino acid sequence of SEQ ID NO: 424, and a CDR-H3 comprising the amino acid sequence of SEQ ID NO: 71, and a VL that comprises a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the VH comprises the amino acid sequence of SEQ ID NO: 75, and the VL comprises the amino acid sequence of SEQ ID NO: 76. In some embodiments, an anti-HER2 antibody comprises a VH that comprises a CDR-H1 comprising the amino acid sequence of SEQ ID NO: 69, a CDR-H2 comprising the amino acid sequence of SEQ ID NO: 70, and a CDR-H3 comprising the amino acid sequence of SEQ ID NO: 71, and a VL that comprises a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74.

In other aspects, the present disclosure provides an anti-CD20 antibody comprising the 6 CDRs and/or VH and VL sequences of any anti-CD20 binding domain provided herein. In some embodiments, an anti-CD20 antibody comprises a VH that comprises a CDR-H1 comprising the amino acid sequence of SEQ ID NO: 556, a CDR-H2 comprising the amino acid sequence of SEQ ID NO: 557, and a CDR-H3 comprising the amino acid sequence of SEQ ID NO: 558, and a VL that comprises a CDR-L1 comprising the amino acid sequence of SEQ ID NO: 559, a CDR-L2 comprising the amino acid sequence of SEQ ID NO: 560, and a CDR-L3 comprising the amino acid sequence of SEQ ID NO: 561. In some embodiments, an anti-CD20 antibody comprises a VH that comprises a CDR-H1 comprising the amino acid sequence of SEQ ID NO: 86, a CDR-H2 comprising the amino acid sequence of SEQ ID NO: 557, and a CDR-H3 comprising the amino acid sequence of SEQ ID NO: 558, and a VL that comprises a CDR-L1 comprising the amino acid sequence of SEQ ID NO: 559, a CDR-L2 comprising the amino acid sequence of SEQ ID NO: 560, and a CDR-L3 comprising the amino acid sequence of SEQ ID NO: 561. In some embodiments, the VH comprises the amino acid sequence of SEQ ID NO: 562, and the VL comprises the amino acid sequence of SEQ ID NO: 563.

One aspect of the present application provides one or more isolated nucleic acids encoding any one of the antibodies, multispecific antibodies, masked antibodies, activatable multispecific antibodies, isolated anti-CD3 antibodies or antigen-binding fragments thereof, masked anti-CD3 antibodies, activatable anti-CD3 antibodies, masked anti-HER2 antibodies or activatable anti-HER2 antibodies described above. In some embodiments, there is provided a vector comprising the one or more nucleic acids according to any one of the nucleic acids described above. In some embodiments, there is provided a host cell comprising the one or more nucleic acids according to any one of the nucleic acids described above or any one of the vectors described above. In some embodiments, there is provided a method for preparing a masked antibody, a multispecific antibody, an activatable multispecific antibody, an isolated anti-CD3 antibody or antigen-binding fragment thereof, a masked anti-CD3 antibody, an activatable anti-CD3 antibody, a masked anti-HER2 antibody or an activatable anti-HER2 antibody, comprising: a) culturing any one of the host cells under conditions that allow expression of the one or more nucleic acids or vector; and b) recovering the multispecific antibody, the activatable multispecific antibody, the anti-CD3 antibody or antigen-binding fragment thereof, the masked anti-CD3 antibody, the activatable anti-CD3 antibody, the masked anti-Her2 antibody, or the activatable antibody from the host cell culture.

Also provided are pharmaceutical compositions comprising any one of the antibodies, multispecific antibodies, masked antibodies, activatable multispecific antibodies, isolated anti-CD3 antibodies or antigen-binding fragments thereof, masked anti-CD3 antibodies, activatable anti-CD3 antibodies, masked anti-HER2 antibodies, or activatable anti-HER2 antibodies described above, and a pharmaceutically acceptable carrier.

Another aspect of the present application provides a method for treating a disease or condition in a subject in need thereof, comprising administering to the subject an effective amount of any one of the pharmaceutical compositions described above. In some embodiments, wherein the pharmaceutical composition comprises an activatable multispecific antibody, wherein the CM1 and the CM2 are cleaved at a diseased site, thereby unblocking binding of the multispecific activatable antibody to CD3 and the target antigen at the diseased site. In some embodiments, the disease or condition is cancer such as liquid cancer and solid cancer. In some embodiments, wherein the target antigen is HER2, the cancer is selected from the group consisting of breast cancer, ovarian cancer, and lung cancer. In some embodiments, wherein the target antigen is CD20, the cancer is lymphoma or leukemia. In some embodiments, the target antigen is TROP2, and wherein the cancer is breast cancer or lymphoma. In some embodiments, the pharmaceutical composition is administered such that the multispecific antibody, isolated antibody or antigen-binding fragment thereof, or masked antibody is provided to the subject at a dose of 0.02 mg/kg, 0.2 mg/kg, 2 mg/kg, 10 mg/kg, 30 mg/kg, or 60 mg/kg. In some embodiments, the multispecific antibody, isolated antibody or antigen-binding fragment thereof, or masked antibody comprises: a first polypeptide comprising an amino acid sequence having at least 90% sequence identity with SEQ ID NO: 427, a second polypeptide comprising an amino acid sequence having at least 90% sequence identity with SEQ ID NO: 428, and a third polypeptide comprising an amino acid sequence having at least 90% sequence identity with SEQ ID NO: 112; a first polypeptide comprising an amino acid sequence having at least 90% sequence identity with SEQ ID NO: 83, a second polypeptide comprising an amino acid sequence having at least 90% sequence identity with SEQ ID NO: 84, and a third polypeptide comprising an amino acid sequence having at least 90% sequence identity with SEQ ID NO: 85; a first polypeptide comprising an amino acid sequence having at least 90% sequence identity with SEQ ID NO: 683, a second polypeptide comprising an amino acid sequence having at least 90% sequence identity with SEQ ID NO: 684, and a third polypeptide comprising an amino acid sequence having at least 90% sequence identity with SEQ ID NO: 685; a first polypeptide comprising the amino acid sequence of SEQ ID NO: 427, a second polypeptide comprising the amino acid sequence of SEQ ID NO: 428, and a third polypeptide comprising the amino acid sequence of SEQ ID NO: 112; a first polypeptide comprising the amino acid sequence of SEQ ID NO: 83, a second polypeptide comprising the amino acid sequence of SEQ ID NO: 84, and a third polypeptide comprising the amino acid sequence of SEQ ID NO: 85; a first polypeptide comprising the amino acid sequence of SEQ ID NO: 683, a second polypeptide comprising the amino acid sequence of SEQ ID NO: 684, and a third polypeptide comprising the amino acid sequence of SEQ ID NO: 685; a first polypeptide comprising the amino acid sequence of SEQ ID NO: 427 without the C-terminal lysine, a second polypeptide comprising the amino acid sequence of SEQ ID NO: 428 without the C-terminal lysine, and a third polypeptide comprising the amino acid sequence of SEQ ID NO: 112; a first polypeptide comprising the amino acid sequence of SEQ ID NO: 83, a second polypeptide comprising the amino acid sequence of SEQ ID NO: 84 without the C-terminal lysine, and a third polypeptide comprising the amino acid sequence of SEQ ID NO: 85 without the C-terminal lysine; or a first polypeptide comprising the amino acid sequence of SEQ ID NO: 683, a second polypeptide comprising the amino acid sequence of SEQ ID NO: 684 without the C-terminal lysine, and a third polypeptide comprising the amino acid sequence of SEQ ID NO: 685 without the C-terminal lysine. In some embodiments, the methods further comprise administering to the subject an anti-PD-1 or anti-PD-L1 antibody. In some embodiments, the methods further comprise administering to the subject a CD137 agonist or antibody. In some embodiments, the CD137 agonist or antibody comprises a heavy chain variable region and a light chain variable region, wherein the heavy chain variable region comprises a CDR-H1 comprising the amino acid sequence of TGGVGVG (SEQ ID NO:700), a CDR-H2 comprising the amino acid sequence of LIDWADDKYYSPSLKS (SEQ ID NO:701), and a CDR-H3 comprising the amino acid sequence of GGSDTVIGDWFAY (SEQ ID NO:702); and/or wherein the light chain variable region comprises a CDR-L1 comprising the amino acid sequence of RASQSIGSYLA (SEQ ID NO:703), a CDR-L2 comprising the amino acid sequence of DASNLET (SEQ ID NO:704), and a CDR-L3 comprising the amino acid sequence of QQGYYLWT (SEQ ID NO:705). In some embodiments, the heavy chain variable region comprises the amino acid sequence of SEQ ID NO:706, and/or wherein the light chain variable region comprises the amino acid sequence of SEQ ID NO:707. In some embodiments, the heavy chain comprises the amino acid sequence of SEQ ID NO:710, and/or the light chain comprises the amino acid sequence of SEQ ID NO:711.

Also provided are compositions, kits and articles of manufacture comprising any one of the multispecific antibodies, masked antibodies, activatable multispecific antibodies, isolated anti-CD3 antibodies or antigen-binding fragments thereof, masked anti-CD3 antibodies, activatable anti-CD3 antibodies, masked anti-HER2 antibodies, or activatable anti-HER2 antibodies described above.

### BRIEF DESCRIPTION OF THE DRAWINGS

FIGS. 1-5 provide schematic diagrams of exemplary antibody designs of the present application. The antibodies may be converted to activatable antibodies by fusing one or more antigen-binding sites to masking peptide(s).

FIG. 1 shows a Fab-Fc/Fc one-armed scaffold schematic.

FIG. 2 shows a schematic of a common light chain scaffold, in which the bispecific antibody has a first antibody heavy chain, a second antibody heavy chain and two copies of a commong light chain. The first antibody heavy chain and a first common light chain form a first antigen-binding site, and the second antibody heavy chain and a second common light chain form a second antigen-binding site. The first antigen-binding site and the second antigen-binding site can bind to different targets.

FIG. 3 shows a schematic of a Morrison format multispecific antibody scaffold, in which the antibody has a first heavy chain fused to a first scFv, a second heavy chain fused to a second scFv and two copies of a common light chain. The first antibody heavy chain and a first common light chain form a first antigen-binding site, and the second antibody heavy chain and a second common light chain form a second antigen-binding site. The first antigen-binding site and the second antigen-binding site can bind to the same target or different targets. The first scFv and the second scFv may bind to the same target or different targets.

FIG. 4 shows ScFv bispecific scaffold schematics. For example, a HER2xCD3 bispecific antibody in this format may have the Fab arm on the left bind to HER2, and the scFv arm on the right bind to CD3.

FIGS. 5A-5B show activatable scaffold schematics. For example, the activatable antibody may be an activatable antibody targeting HER2 and CD3 (HER2xCD3 activatable antibody or SAFEbody) or an antivatable antibody targeting CD20 and CD3 (CD20xCD3 activatable antibody or SAFEbody). The masking peptide (represented as a ball) can be fused to the antigen-binding fragment via a cleavable linker.

FIG. 6 provides a characterization of bispecific antibodies through SDS-PAGE electrophoresis. The left gel is a 12% SDS-PAGE gel under reducing conditions, and the right gel is a 4-15% SDS-PAGE gel under non-reducing conditions. The MW lane shows molecular weight markers, which are labeled in kilodaltons to the left of each gel. In both gels, lane 1 shows antibody TY24051, lane 2 shows antibody TY24052, and lane 3 shows antibody TY24053.

FIG. 7 provides size-exclusion high-performance liquid chromatography analyses of bispecific antibodies. The upper plot shows antibody TY24051, the middle plot shows antibody TY24105, and the lower plot shows antibody TY24106. In each plot, time is on the x-axis, and relative protein abundance is on the y-axis. Peaks corresponding to heterodimeric proteins (major peak), homodimeric proteins, and aggregates are indicated.

FIGS. 8A-8B provide enzyme-linked immunosorbent assay (ELISA) analyses of antibodies TY24051 and TY24052. FIG. 8A shows binding of HER2 by TY24051 (squares), TY24052 (triangles pointing up), and TY24052 after activation (triangles pointing down). FIG. 8B shows binding of CD3 by TY24051 (squares), TY24052 (triangles pointing up), and TY24052 after activation (triangles pointing down). In both FIG. 8A and FIG. 8B, the concentration of antibody is on the x-axis in M, and the absorbance at 450 nm is on the y-axis.

FIG. 9 shows an assay of T-cell mediated cytotoxic killing upon treatment with bispecific antibodies. The concentration of antibody (ng/ml) is shown on the x-axis, and the percentage of cell lysis is shown on the y-axis. Target cells were incubated with T cells for 24 hours with TY24051 (circles), TY24052 (squares), an isotype control (triangles pointing up), or without an antibody (triangles pointing down).

FIGS. 10A-10B show activation of a nuclear factor of activated T-cells (NFAT) response element reporter in Jurkat cells in response to treatment with bispecific antibodies TY24051 (black circles), TY24111 (squares), TY24052 (white circles), and TY24110 (triangles). The log-transformed concentration of antibody in μg/ml is indicated on the x-axis, and relative light units (RLU) of the reporter are indicated on the y-axis.

In FIG. 10A, NFAT reporter activity was measured in the absence of target (SK-OV-3) cells.

In FIG. 10B, NFAT reporter activity was measured in the presence of target cells.

FIG. 11 shows secreted IFNγ levels following administration of parental (TAC2245) or activatable (TY23104) anti-CD3 antibodies in a humanized peripheral blood mononuclear cell (PBMC) mouse model (huPBMC-NSG). The identity of the antibody and the time of sampling are indicated on the x-axis, including, from left to right, a blank, TAC2245 sampled after 0 hours of treatment, TAC2245 sampled after 3 hours of treatment, TAC2245 sampled after 24 hours of treatment, TY23104 sampled after 0 hours of treatment, TY23104 sampled after 3 hours of treatment, and TY23104 sampled after 24 hours of treatment. The y-axis shows the concentration of IFNγ in picograms/ml.

FIG. 12 shows secreted IFNγ levels following administration of parental (TAC2245) or activatable (TY23115 and TY23118) cross-reactive anti-CD3 antibodies in a huPBMC-NSG mouse model. The identity of the antibody and the time of sampling are indicated on the x-axis, including, from left to right, a blank, TAC2245 sampled after 0 hours of treatment, TAC2245 sampled after 3 hours of treatment, TAC2245 sampled after 24 hours of treatment, TY23115 sampled after 0 hours of treatment, TY23115 sampled after 3 hours of treatment, TY23115 sampled after 24 hours of treatment, TY23118 sampled after 0 hours of treatment, TY23118 sampled after 3 hours of treatment, and TY23118 sampled after 24 hours of treatment. The y-axis shows the concentration of IFNγ in picograms/ml.

FIG. 13 shows the level of Jurkat cell binding by parental anti-CD3 antibody TAC2245 (circles) and activatable anti-CD3 antibody TY23104 (squares). The log-transformed concentration of anti-CD3 antibody in nM is indicated on the x-axis, and mean fluorescence intensity (MFI) of the binding of a secondary anti-human IgG antibody is indicated on the y-axis.

FIG. 14 shows activation of a NFAT response element reporter in Jurkat cells in response to treatment with parental (TAC2225, circles) or activatable (TY23115, squares; and TY23118, triangles) cross-reactive anti-CD3 antibodies. The log-transformed concentration of antibody in nM is indicated on the x-axis, and the relative light units (RLU) of the reporter is indicated on the y-axis.

FIG. 15 shows activation of a NFAT response element reporter in Jurkat cells in response to treatment with parental (TAC2245, circles), or activatable (TY23100, black squares; TY23101, triangles pointing up; TY23102, triangles pointing down; and TY23104, white squares) anti-CD3 antibodies. The log-transformed concentration of antibody in μg/mL is indicated on the x-axis, and the relative light units (RLU) of the reporter is indicated on the y-axis. The assay was performed without FcRIIb crosslinking.

FIGS. 16A-16B show analyses of the masking efficiencies of parental and activatable anti-CD3 antibodies. FIG. 16A shows binding of parental (TAC2225, black circles) and activatable anti-CD3 antibodies (TY23110, squares; TY23115, triangles pointing up; and TY23118, triangles pointing down) to recombinant human CD3&8 as determined by an ELISA.

FIG. 16B shows activation of a NFAT response element reporter in Jurkat cells in response to treatment with parental (TAC2225, black circles) and activatable anti-CD3 antibodies (TY23105, white circles; TY23110, squares; TY23115, triangles pointing up; and TY23118, triangles pointing down). The log-transformed concentration of antibody in μg/mL is indicated on the x-axis, and the relative light units (RLU) of the reporter is indicated on the y-axis.

FIG. 17 shows activation of a NFAT response element reporter in Jurkat cells in response to treatment with parental (TAC2225, white circles), or activatable anti-CD3 antibodies. In each graph of FIG. 17, the log-transformed concentration of antibody in μg/mL is indicated on the x-axis, the relative light units (RLU) of the reporter is indicated on the y-axis, and the identities of the activatable anti-CD3 antibodies are indicated by the shape of the data points, as shown in each legend. Assays that were performed without FcRIIb crosslinking are indicated.

FIG. 18 shows the level of Jurkat cell binding by parental anti-CD3 antibody TAC2245 (TAC2225, circles) and activatable anti-CD3 antibodies. In each graph of FIG. 18, the log-transformed concentration of anti-CD3 antibody in nM is indicated on the x-axis, mean fluorescence intensity (MFI) of the binding of a secondary anti-human IgG antibody is indicated on the y-axis, and the identities of the activatable anti-CD3 antibodies are indicated by the shape of the data points, as shown in each legend.

FIG. 19 shows binding of parental and activatable anti-CD3 antibodies to recombinant human CD3&8 as determined by an ELISA. The log-transformed concentration of antibody in M is indicated on the x-axis, the absorbance at a wavelength of 450 nm is indicated on the y-axis, and the identities of the anti-CD3 antibodies are indicated by the shape of the data points, as shown in the legend.

FIGS. 20A-20B show analyses of the masking efficiencies of parental and activatable SP34 variant anti-CD3/HER2 bispecific antibodies.

FIG. 20A shows binding of parental (TY25023, black circles) and activatable (TY25026, white circles) antibodies with low-anti-CD3 affinity, and comparison parental (TY24051, black squares) and activatable (TY24052, white squares) antibodies to recombinant human CD368, as determined by ELISA. The log-transformed concentration of antibody in M is indicated on the x-axis, and the absorbance at a wavelength of 450 nm is indicated on the y-axis.

FIG. 20B shows the level of Jurkat cell binding by anti-CD3 antibodies TY24051 (black circles), TY24052 (white circles), and TY25023 (black squares). The log-transformed concentration of antibody in nM is indicated on the x-axis, and mean fluorescence intensity (MFI) of the binding of a secondary anti-human IgG antibody is indicated on the y-axis.

FIGS. 21A-21C show analyses of the masking efficiencies and functions of parental and activatable SP34 variant anti-CD3/HER2 bispecific antibodies.

FIG. 21A shows activation of a NFAT response element reporter in Jurkat cells in response to treatment with bispecific antibodies TY24051 (black circles), TY24052 (white circles), TY25023 (black squares), and TY25026 (white squares). The log-transformed concentration of antibody in μg/ml is indicated on the x-axis, and relative light units (RLU) of the reporter are indicated on the y-axis. In FIG. 21A, NFAT reporter activity was measured in the presence of target (SK-OV-3) cells.

FIG. 21B shows the level of SK-OV3 tumor cell lysis in response to treatment with bispecific antibodies TY24051 (dark gray circles), TY24052 (dark gray squares), TY25023 (light gray triangles), TY25026 (light gray squares), and a reference CD3× isotype control (dark gray triangles). The log-transformed concentration of antibody in ng/mL is indicated on the x-axis, and % cytotoxicity is indicated on the y-axis. The EC₅₀ of cytotoxicity is indicated for each antibody in ng/mL in the table below the plot.

FIG. 21C shows secreted IFNγ levels in an activated CD8+ T cell assay in response to treatment with bispecific antibodies TY24051 (dark gray squares), TY24052 (dark gray circles), TY25023 (light gray squares), and TY25026 (light gray circles). The log-transformed concentration of antibody in nM is indicated on the x-axis, and concentration of IFNγ in picograms/mL is indicated on the y-axis.

FIGS. 22A-22B show cytokine release in cynomolgus monkeys treated with parental or activatable bispecific antibodies.

FIG. 22A shows the level of cytokines IFNγ, IL-2, IL-6, TNFα, IL-5, and IL-4 released in response to treatment with bispecific antibodies TY24051 (dark gray squares), TY24052 (dark gray circles), TY25023 (light gray squares), and TY25026 (light gray circles). The x-axis shows the time following administration in hours, and the y-axis shows the concentration of the cytokine in picograms/mL. The points in time at which 0.2, 0.5, and 0.9 mg/kg (“mpk”) doses of antibody were administered are indicated above each plot with arrows. The level of IL-6 release is also provided in FIG. 24F, with a log-transformed y-axis.

FIG. 22B shows the level of cytokines IFNγ, IL-2, IL-6, TNFα, IL-5, and IL-4 released in response to treatment with bispecific antibodies TY24051, TY24052, TY25023, and TY25026. The x-axis shows the time following administration in hours, and the y-axis shows the log-transformed concentration of the cytokine in picograms/mL. The points in time at which 0.2, 0.5, and 0.9 mg/kg doses of antibody were administered are indicated above each plot with arrows. The level of IL-6 release is also provided in FIG. 24F, with a log-transformed y-axis.

FIG. 23 shows the level of CD4+ (left plot) and CD8+ (right plot) T cell activation in response to treatment with bispecific antibodies TY24051 (dark gray squares), TY24052 (dark gray circles), TY25023 (light gray squares), and TY25026 (light gray circles). The x-axis shows the time following administration in hours, and the y-axis shows the percentage of CD69+ T cells. The points in time at which 0.2, 0.5, and 0.9 mg/kg (“mpk”) doses of antibody were administered are indicated above each plot with arrows.

FIGS. 24A-24F show results from a study of cynomolgus monkeys treated with parental or activatable bispecific antibodies.

FIG. 24A shows the level of T cells per μL for total T cells (top), CD4+ T cells (bottom, left), and CD8+ T cells (bottom, right) in monkeys in response to treatment with bispecific antibodies TY24051 (dark gray squares), TY24052 (dark gray circles), TY25023 (light gray squares), and TY25026 (light gray circles). The x-axis shows the time following administration in hours, and the y-axis shows the number of cells per μL. The points in time at which 0.2, 0.5, and 0.9 mg/kg (“mpk”) doses of antibody were administered are indicated above each plot with arrows.

FIG. 24B shows the level of B cells (left) and NK cells (right) per μL in monkeys in response to treatment with bispecific antibodies TY24051 (circles), TY24052 (squares), TY25023 (triangles pointing up), and TY25026 (triangles pointing down). The x-axis shows the time following administration in hours, and the y-axis shows the number of cells per μL. The points in time at which 0.2, 0.5, and 0.9 mg/kg (“mpk”) doses of antibody were administered are indicated above each plot with arrows.

FIG. 24C shows the level of bispecific antibodies TY24051 (circles), TY24052 (squares), TY25023 (triangles pointing up), and TY25026 (triangles pointing down) in cynomolgus monkeys. The x-axis shows the time following administration in hours, and the y-axis shows the log-transformed concentration of antibody in μg/mL. The points in time at which 0.2, 0.5, and 0.9 mg/kg (“mpk”) doses of antibody were administered are indicated above each plot with arrows.

FIG. 24D shows plasma concentrations of bispecific antibodies and pharmacokinetics parameters in monkeys treated with bispecific antibodies.

FIG. 24E shows IL-6 release in monkeys treated with bispecific antibodies. The parental bispecific antibody is shown in squares, and the activatable bispecific antibody is shown in circles.

FIG. 24F shows absolute lymphocyte count in monkeys treated with bispecific antibodies. The parental bispecific antibody is shown in squares, and the activatable bispecific antibody is shown in circles.

FIGS. 25A-25B provide a flow cytometry analysis of yeast cell surface display of anti-HER2 antibodies. In each scatterplot of FIGS. 25A-25B, the x-axis shows the level of Fab or scFv displayed on the yeast cell (detected by the binding of an antibody to the affinity tag fused to the anti-HER2 antibody), and the y-axis indicates the level of HER2-binding (detected by the binding of PE conjugated streptavidin to biotinylated human HER2-Fc).

FIG. 25A shows the binding of Fabs to HER2.

FIG. 25B shows the binding of scFvs to HER2.

FIG. 26 shows the results of four rounds (R1, R2, R3, and R4) of FACS to screen a CPL yeast library for masking peptides to mask binding to 10 nM of biotinylated HER2-Fc. In each scatterplot of FIG. 26, the x-axis indicates the level of myc-tagged anti-HER2 antibody, and the y-axis indicates the level of indicates the level of HER2-binding.

FIGS. 27A-27B show FACS analyses of binding of the selected trastuzumab-derived activatable anti-HER2 antibodies. In each scatterplot of FIGS. 27A-27B, samples were treated with the buffer PBSA (left) or TEV protease (right), the x-axis shows the level of Fab or scFv displayed on the yeast cell (detected by the binding of an antibody to the affinity tag fused to the anti-HER2 antibody), and the y-axis indicates the level of HER2-binding (detected by the binding of PE conjugated streptavidin to biotinylated human HER2-Fc).

In FIG. 27A, the anti-HER2 antibody (B14126) is in the scFv format.

In FIG. 27B, the anti-HER2 antibody (B14132) is in the Fab format.

FIG. 28 shows a Biolayer Interferometry analysis of binding of parental (trastuzumab) and activatable anti-HER2 antibodies (TY22841, TY22842, TY22839, TY22838, and TY22837) to His-tagged HER2, as a measurement of the masking efficiency of the activatable antibodies.

The x-axis indicates time in seconds, and the y-axis indicates the level of binding.

FIGS. 29A-29C show binding of parental (trastuzumab, black circles) and activatable anti-HER2 antibodies to recombinant HER2-Fc, as determined by an ELISA. The log-transformed concentration of antibody in M is indicated on the x-axis, and the absorbance at a wavelength of 450 nm is indicated on the y-axis.

FIG. 29A shows results for TY22836, TY2237, TY2238, TY2239, TY2240, TY2241, TY2242, TY2243, and trastuzumab.

FIG. 29B shows results for TY22846, TY2247, TY2250, TY2251, TY2252, TY2253, TY2254, and trastuzumab.

FIG. 29C shows results for TY23523, TY23525, TY23526, TY23533, TY23536, TY23537, and trastuzumab.

FIG. 30 provides reduced a SDS-PAGE showing TY22837 alone (lane 1) or in the presence of the protease MMP-9 (lane 2).

FIG. 31 shows binding of parental anti-HER2 antibody (trastuzumab, black circles) and TY22837 to recombinant HER2-Fc, as determined by an ELISA. TY22837 binding is shown for TY22837 alone (triangles pointing down) or in the presence of the protease MMP-9 (triangles pointing up). The log-transformed concentration of antibody in M is indicated on the x-axis, and the absorbance at a wavelength of 450 nm is indicated on the y-axis.

FIG. 32 shows the level of SK-OV-3 cell binding by parental (trastuzumab, black circles) and activatable (TY22837, white circles; TY23536, squares) anti-HER2 antibodies. The log-transformed concentration of antibody in nM is indicated on the x-axis, and mean fluorescence intensity (MFI) of the binding of a secondary anti-human IgG antibody is indicated on the y-axis.

FIGS. 33A-33C show the results of three stress tests of activatable anti-HER2 antibodies TY22837 (left column) and TY22838 (right column). In each of FIGS. 33A-33C, the x-axis shows time in minutes, and the y-axis shows the level of antibody aggregation, as indicated by absorbance units at 214 nm.

FIG. 33A shows results after the activatable antibodies underwent three or six freeze-thaw cycles.

FIG. 33B shows results after incubation of the activatable antibodies at 50° C. for 7 days.

FIG. 33C shows results after incubation of the activatable antibodies at 40° C. for 28 days.

FIGS. 34A-34B show binding of parental (trastuzumab) and activatable anti-HER2 antibodies to recombinant HER2-Fc, as determined by an ELISA. The length of the masking peptides of the activatable antibodies was modified, as shown in Table 19. In each of FIGS. 34A-34B, the log-transformed concentration of antibody in M is indicated on the x-axis, and the absorbance at a wavelength of 450 nm is indicated on the y-axis.

FIG. 34A shows the results for trastuzumab (circles), TY23171 (triangles pointing up), TY23172 (triangles pointing down), and TY22836 (squares).

FIG. 34B shows the results for trastuzumab (circles), TY23173 (squares), TY23174 (triangles pointing down), and TY22837 (triangles pointing down).

FIGS. 35A-35C show lymphocyte counts, T cell activation, and pharmacokinetic parameters in cynomolgus monkeys treated with the CD3 masked only bispecific antibody TY25362.

FIG. 35A shows the level of cells per μL for total T cells (top, left), CD4+ T cells (top, center), CD8+ T cells (top, right), B cells (bottom, left), and NK cells (bottom, right) in monkeys in response to treatment with the CD3 masked only bispecific antibody TY25362. The x-axis shows the time following administration in hours, and the y-axis shows the number of cells per μL. The points in time at which 1, 10, and 30 mg/kg (“mpk”) doses of antibody were administered are indicated above each plot with arrows.

FIG. 35B shows the level of CD4+(left plot) and CD8+(right plot) T cell activation in response to treatment with bispecific antibody TY25362. The x-axis shows the time following administration in hours, and the y-axis shows the percentage of CD69+ T cells. The points in time at which 1, 10, and 30 mg/kg (“mpk”) doses of antibody were administered are indicated above each plot with arrows.

FIG. 35C shows the level of TY25362 in cynomolgus monkeys. The x-axis shows the time following administration in hours, and the y-axis shows the log-transformed concentration of antibody in μg/mL. The points in time at which 1, 10, and 30 mg/kg (“mpk”) doses of antibody were administered are indicated above each plot with arrows.

FIGS. 36A-36E show binding affinity measurements of TY25023 and TY24051 to CD3.

FIG. 36A shows the EC₅₀ and Kd of TY25023 and TY24051 binding to human or monkey CD3δε as determined by an ELISA or Biacore interferometry, respectively.

FIG. 36B shows binding of TY25023 and TY24051 to human CD3δε as determined by ELISA. The EC₅₀ of binding human CD3δε is indicated for each antibody in nM in the table to the right of the plot.

FIG. 36C shows binding of TY25023 and TY24051 to monkey CD3δε as determined by ELISA. The EC₅₀ of binding monkey CD3δε is indicated for each antibody in nM in the table to the right of the plot.

FIG. 36D shows binding of TY25023 and TY24051 to human CD3δε as determined using Biacore interferometry.

FIG. 36E shows binding of TY25023 and TY24051 to monkey CD3δε as determined using Biacore interferometry.

FIGS. 37A-37D show the results of cytokine release assays in cynomolgus monkeys treated with parental or activatable anti-CD3 and anti-CD20 bispecific antibodies, as measured by ELISA.

FIG. 37A shows the level of IL-2 in cynomolgus monkey serum over time. The x-axis shows the time following administration in hours, and the y-axis shows the level of IL-2 in pg/mL. The point in time at which the 0.3 mg/kg dose of antibody was administered is indicated with an arrow. TY25455 is shown as circles, TY25606 is shown as squares, TY25715 is shown as triangles pointing up, and TY25816 is shown as triangles pointing down.

FIG. 37B shows the peak level of IL-2 in cynomolgus monkey serum. The x-axis indicates the identity of the antibody, and the y-axis shows the peak level of IL-2 in pg/mL.

FIG. 37C shows the level of IFN-γ in cynomolgus monkey serum over time. The x-axis shows the time following administration in hours, and the y-axis shows the level of IFN-γ in pg/mL. The point in time at which the 0.3 mg/kg dose of antibody was administered is indicated with an arrow. TY25455 is shown as circles, TY25606 is shown as squares, TY25715 is shown as triangles pointing up, and TY25816 is shown as triangles pointing down.

FIG. 37D shows the peak level of IFN-γ cynomolgus monkey serum. The x-axis indicates the identity of the antibody, and the y-axis shows the peak level of IFN-γ in pg/mL.

FIGS. 38A-38C show measurements of pharmacodynamics markers in cynomolgus monkeys treated with parental or activatable anti-CD3 and anti-CD20 bispecific antibodies, measured using FACS.

FIG. 38A shows lymphocyte (top left), CD3+ T cell (top right), and CD19+ B cell (bottom left) counts over the first 24 hours following antibody administration. In each plot, the x-axis shows the time following administration in hours, and the y-axis shows the cell count in ×10⁹ cells/L. The points in time at which the 0.3 mg/kg dose of antibody was administered is indicated with an arrow. TY25455 is shown as circles, TY25606 is shown as squares, TY25715 is shown as triangles pointing up, and TY25816 is shown as triangles pointing down.

FIG. 38B shows lymphocyte (top left), CD3+ T cell (top right), and CD19+ B cell (bottom left) counts over 14 days following antibody administration. In each plot, the x-axis shows the time following administration in hours, and the y-axis shows the cell count in ×10⁹ cells/L. The points in time at which the 0.3 mg/kg dose of antibody was administered is indicated with an arrow. TY25455 is shown as circles, TY25606 is shown as squares, TY25715 is shown as triangles pointing up, and TY25816 is shown as triangles pointing down.

FIG. 38C shows CD3+CD8+ T cell (top left), CD3+CD4+ T cell (top right), CD8+CD69+ T cell (bottom left), and CD4+CD69+ T cell (bottom right) counts over 14 days following antibody administration. In each plot, the x-axis shows the time following administration in hours, and the y-axis shows the percentage of cells vs. the level of lymphocytes. The points in time at which the 0.3 mg/kg dose of antibody was administered is indicated with an arrow. TY25455 is shown as circles, TY25606 is shown as squares, TY25715 is shown as triangles pointing up, and TY25816 is shown as triangles pointing down.

FIGS. 39A-39B show measurements of pharmacodynamics markers in cynomolgus monkeys treated with the activatable anti-CD3 and anti-CD20 bispecific antibody TY25606, measuring using FACS.

FIG. 39A shows lymphocyte (top left), CD3+ T cell (top right), and CD19+ B cell (bottom left) counts over 50 days following antibody administration. In each plot, the x-axis shows the time following administration in hours, and the y-axis shows the cell count in x10⁹ cells/L. The points in time at which the 0.3 and 1 mg/kg doses of antibody were administered are indicated with arrows.

FIG. 39B shows CD3+CD8+ T cell (top left), CD3+CD4+ T cell (top right), CD8+CD69+ T cell (bottom left), and CD4+CD69+ T cell (bottom right) counts over 50 days following antibody administration. In each plot, the x-axis shows the time following administration in hours, and the y-axis shows the percentage of cells vs. the level of lymphocytes. The points in time at which the 0.3 and 1 mg/kg doses of antibody were administered are indicated with arrows.

FIG. 40 shows the level of total human IgG in cynomolgus monkeys treated with the activatable anti-CD3 and anti-CD20 bispecific antibody TY25606, measured using FACS. The x-axis shows the time following administration in hours, and the y-axis shows the log-transformed level of total human IgG in μg/mL. The points in time at which the 0.3 and 1 mg/kg doses of antibody were administered are indicated with arrows.

FIGS. 41A-41B show the effect of parental or activatable anti-CD3 and anti-CD20 bispecific antibodies on a reporter assay with or without Raji tumor cells.

FIG. 41A shows the reporter assay with Raji tumor cells. The x-axis shows the log-transformed concentration of antibody in nM, and the y-axis shows the relative luminescence units (“RLU”) of the reporter. The gray area represents the calculated peak concentration in cynomolgus serum at the 0.3 mg/kg dosage. TAC2392 is shown as black circles, TAC2415 is shown as white circles, TY25455 is shown as black squares, TY25606 is shown as white squares, TY25715 is shown as triangles pointing up, TY25816 is shown as triangles pointing down, and an isotype control is shown as diamonds.

FIG. 41B shows the reporter assay without Raji tumor cells. The x-axis shows the log-transformed concentration of antibody in nM, and the y-axis shows the relative luminescence units (“RLU”) of the reporter. TAC2392 is shown as black circles, TAC2415 is shown as white circles, TY25455 is shown as black squares, TY25606 is shown as white squares, TY25715 is shown as triangles pointing up, TY25816 is shown as triangles pointing down, and an isotype control is shown as diamonds.

FIGS. 42A-42B show the effect of parental or activatable anti-CD3 and anti-CD20 bispecific antibodies on a reporter assay with or without SU-DHL-4 tumor cells.

FIG. 42A shows the reporter assay with SU-DHL-4 tumor cells. The x-axis shows the log-transformed concentration of antibody in nM, and the y-axis shows the relative luminescence units (“RLU”) of the reporter. TAC2392 is shown as black circles, TAC2415 is shown as white circles, TY25455 is shown as black squares, TY25606 is shown as white squares, TY25715 is shown as triangles pointing up, TY25816 is shown as triangles pointing down, and an isotype control is shown as diamonds. The gray area represents the calculated peak concentration in cyno serum at the 0.3 mg/kg dosage.

FIG. 42B shows the reporter assay without SU-DHL-4 tumor cells. The x-axis shows the log-transformed concentration of antibody in nM, and the y-axis shows the relative luminescence units (“RLU”) of the reporter. TAC2392 is shown as black circles, TAC2415 is shown as white circles, TY25455 is shown as black squares, TY25606 is shown as white squares, TY25715 is shown as triangles pointing up, TY25816 is shown as triangles pointing down, and an isotype control is shown as diamonds.

FIGS. 43A-43B show the effect of parental or activatable anti-CD3 and anti-CD20 bispecific antibodies on an in vitro B cell killing assay, using PBMCs.

FIG. 43A shows the level of endo B cell killing. The x-axis shows the log-transformed concentration of antibody in nM, and the y-axis shows the percentage of human endo B cell killing. AC1281 is shown as black circles, TAC2415 is shown as white circles, TY25455 is shown as black squares, TY25606 is shown as white squares, TY25715 is shown as triangles pointing up, TY25816 is shown as triangles pointing down, and an isotype control is shown as diamonds. Below the plot, the EC₅₀ of B cell killing for each antibody is shown in nM.

FIG. 43B shows the level of CD8+ T cell activation. The x-axis shows the log-transformed concentration of antibody in nM, and the y-axis shows the percentage of CD69+ cells in the CD8+ T cell population. TAC2392 is shown as black circles, TAC2415 is shown as white circles, TY25455 is shown as black squares, TY25606 is shown as white squares, TY25715 is shown as triangles pointing up, TY25816 is shown as triangles pointing down, and an isotype control is shown as diamonds. Below the plot, the EC₅₀ of T cell activation for each antibody is shown in nM.

FIG. 44 shows the level of T and B cell binding to antibodies TAC2392 (black circles), TY2455 (black triangles pointing down), and an isotype control (white circles) as measured using FACS, using PBMCs. In each plot, the x-axis shows the log-transformed concentration of antibody in nM, and the y-axis shows the level of binding, as mean fluorescence intensity (“MFI”). Binding to human CD4+ T cells is shown on the upper left, binding to human CD8+ T cells is shown in the upper center, binding to human B cells is shown on the upper right, binding to monkey CD4+ T cells is shown on the lower left, binding to monkey CD8+ T cells is shown on the lower center, and binding to monkey B cells is shown on the lower right. Below the plots, the EC₅₀ of TAC2392 and TY2455 binding to each cell type is shown in nM.

FIG. 45 shows tumor volume over time in female M-NSG immunodeficient mice with human PBMCs and EMT6 mouse breast cancer cells stably transfected with HER2. The mice were administered 5 mg/kg of the antibodies TY24051 (black circles), TY25023 (triangles pointing up), TY25026 (squares), TY25362 (triangles pointing down), and an isotype control (white circles). The x-axis indicates the number of days post inoculation, with the points in time at which doses of antibody were administered indicated with arrows, and the y-axis shows tumor volume in mm³.

FIG. 46 shows a schematic diagram of a proposed SAFEbody mechanism of action. As shown at left, when a SAFEbody is in proximity to normal tissues (e.g., tissues lacking an epitope bound by the SAFEbody), the SAFEbody remains masked. Without wishing to be bound by theory, two paths are envisioned for the mechanism by which a SAFEbody binds a target site. In path 1, a cleavable moiety is cleaved by a protease in proximity to the tumor tissue, thereby removing the masking moiety and unmasking the SAFEbody so that it can bind the target. In path 2, the cleavable moiety is not necessarily cleaved, and binding of the SAFEbody for the target is in competition for binding of the SAFEbody to the masking moiety. Upon binding of the SAFEbody to the target site, the cleavable moiety can be cleaved by a protease, thereby unmasking the SAFEbody.

FIG. 47 shows induction of luciferase expression in Jurkat/NFAT-Luc reporter line by the CD20xCD3 bispecific antibodies in the presence of target Raji cells used to screen additional CD20xCD3 bispecific antibodies.

FIG. 48 shows tumor growth curves of different treatment groups (N=6) of female M-NSG mice bearing Raji-established tumors.

FIGS. 49A-49B show PK study of TY25455 and TY25606 on tumor-bearing mice. FIG. 49A shows the concentration of TY25455 in tumor-bearing mice at different time points with different dosing strategies. FIG. 49B shows the concentration of TY25606 in tumor-bearing mice at different time points with different dosing strategies.

FIGS. 50A-50D show cynomolgus monkey toxicity and pharmacology studies of single dose injection of the CD20xCD3 bispecific or SAFEbody/bispecific antibodies. FIG. 50A shows a plot of normalized CD19⁺B cell percentage over time in blood samples from cynomolgus monkeys treated with a single dose of drugs. FIG. 50B shows a plot of normalized CD3+ T cell percentage over time in blood samples from cynomolgus monkeys treated with a single dose of drugs. FIG. 50C shows the pre-dose and post-dose levels (pg/mL) of IFN-γ for cynomolgus monkeys treated with a single dose of drugs. FIG. 50D shows the pre-dose and post-dose levels (pg/mL) of IL-2 for cynomolgus monkeys treated with a single dose of drugs.

FIGS. 51A-51C show the binding affinities of the HER2xCD3 bispecific antibodies to CD3 and HER2 as determined by enzyme-linked immunosorbent assays (ELISAs). FIG. 51A shows the CD3δε ELISA binding curves of bispecific antibodies TY24051, TY25238 and TY25023. FIG. 51B shows the CD3 δε ELISA binding curves of bispecific antibody TY25238 and activatable antibodies TY27151 and TY27008. FIG. 51C shows the HER2 ELISA binding curves of trastuzumab, bispecific antibody TY25238 and activatable antibodies TY27151 and TY27008.

FIGS. 52A-52C show results of killing assays of SKOV3 (FIG. 52A), MCF7 (FIG. 52B) and A549 cells (FIG. 52C) by CD8+ T cells in the presence of bispecific antibodies TY25023, TY24051, and TY25238.

FIGS. 53A-53B show cleavage efficiencies of the masking moieties on the anti-CD3 (FIG. 53A) and anti-HER2 (FIG. 53B) antibody moieties in various HER2xCD3 bispecific antibodies.

FIGS. 54A-54B show in vivo anti-tumor efficacy of the HER2xCD3 antibodies and negative control in HER2 expressing tumors (SK-OV3) in a xenogeneic in vivo tumor model. Data points represent group mean; error bars represent SEM.

FIGS. 55A-55C show PK data in cynomolgus monkeys treated with HER2xCD3 bispecific antibodies. FIG. 55C shows systemic cytokine release (IL-6, IFN-γ, IL-2, and TNF-α) in cynomolgus monkeys.

FIGS. 56A-56B show in vitro cytokine release, including IFN-γ (FIG. 56A) or IL-2 (FIG. 56B) by human PBMCs in the presence of MCF7.

FIGS. 57A-57B show in vivo anti-tumor efficacy of the HER2xCD3 antibodies and negative control in HER2 expressing tumors (SK-OV3) in a xenogeneic in vivo tumor model. Data points represent group mean; error bars represent SEM.

FIG. 58 shows lymphocytes margination induced by TY25023, TY25026 and TY25362.

FIG. 59 shows cytokine release levels in cynomolgus monkeys administered with TY25023, TY25026 and TY25362 as determined by ELISA.

FIG. 60 shows PK curves in cynomolgus monkeys administered with TY25023, TY25026 and TY25362.

FIG. 61 shows the results of a luciferase-based CD3 gene reporter assay characterizing effect of anti-HER2xCD3 activatable/bispecific antibodies on activation of CD3 signaling.

FIG. 62 shows in vivo anti-tumor efficacy of anti-HER2xCD3 bispecific parental antibody TY25238 and activatable antibodies TY27008 and TY27151 in PBMC-engrafted HT55 xenograft model. Data points represent group mean; error bars represent SEM. Antibody dosing is denoted by arrows.

FIG. 63 shows in vivo anti-tumor efficacy of anti-HER2xCD3 bispecific activatable antibody TY27151 in PBMC-engrafted HT55 xenograft model, as compared to trastuzumab, DS-8201 ADC, or vehicle. Data points represent group mean; error bars represent SEM. Antibody dosing is denoted by arrows.

FIGS. 64A & 64B show synergistic anti-tumor efficacy of anti-HER2xCD3 bispecific activatable antibody TY27151 in combination with anti-CD137 mAb in an MC38-hHER2 murine colon cancer syngeneic model. FIG. 64A shows in vivo anti-tumor efficacy of anti-HER2xCD3 bispecific activatable TY27151 in the MC38-hHER2 murine colon cancer syngeneic model. Antibody dosing is denoted by arrows. FIG. 64B shows results of MC38-hHER2 tumor rechallenge without further antibody treatment. Arrow indicates tumor re-challenge. In both figures, data points represent group mean; error bars represent SEM.

FIG. 65 shows in vivo anti-tumor efficacy of anti-HER2xCD3 bispecific activatable antibody TY27151 administered as a monotherapy or in combination with the anti-PD-1 mAb 2E5 in PBMC-engrafted SK-OV3 xenograft model. Data points represent group mean; error bars represent SEM. Antibody dosing is denoted by arrows.

### DETAILED DESCRIPTION

The present application provides masked multispecific antibodies comprising a first antigen-binding fragment that specifically binds CD3 with weak affinity and a second antigen-binding fragment that specifically binds a target antigen, wherein the first antigen-binding fragment is fused to a first masking moiety. The masking moiety may be fused to the first antigen-binding fragment via a cleavable linker or a non-cleavable linker. Without wishing to be bound by theory, it is believed that a multispecific antibody comprising a first masking moiety can be in a state of dynamic equilibrium between a masked state in which the antigen-binding fragment that specifically binds CD3 is bound to the masking moiety, and a CD3-bound state in which the antigen-binding fragment that specifically binds CD3 is bound to CD3. Accordingly, the relative binding affinities of the masking moiety for the antigen-binding fragment and the antigen-binding fragment for CD3 determine the extent to which the antibody actually engages CD3. Due to the weak affinity of the first antigen-binding fragment and the high masking efficiency of the first masking moiety, the multispecific antibodies described herein provide a wide therapeutic window and reduce side effects associated with non-specific binding. The multispecific antibodies described herein provide a safe and effective therapeutic approach for treatment of various diseases and conditions, including liquid and solid cancer that is associated with the target antigen.

Accordingly, one aspect of the present application provides a multispecific antibody comprising: a) a first antigen-binding fragment that specifically binds CD3, wherein the first antigen-binding fragment is fused to a first masking moiety (MM1); and b) a second antigen-binding fragment that specifically binds a target antigen; wherein the MM1 competes with CD3 to specifically bind the first antigen-binding fragment; and wherein the first antigen-binding fragment binds CD3 with half-maximal binding at a concentration of antibody (EC₅₀) that is at least 10 nM (e.g., at least 100 nM) as determined by an enzyme-linked immunosorbent assay (ELISA, such as the ELISA assay of Example 3). In some embodiments, the MM1 comprises an amino acid sequence of SEQ ID NO: 35 or 417. In some embodiments, the target antigen is HER2. In some embodiments, the target antigen is CD20.

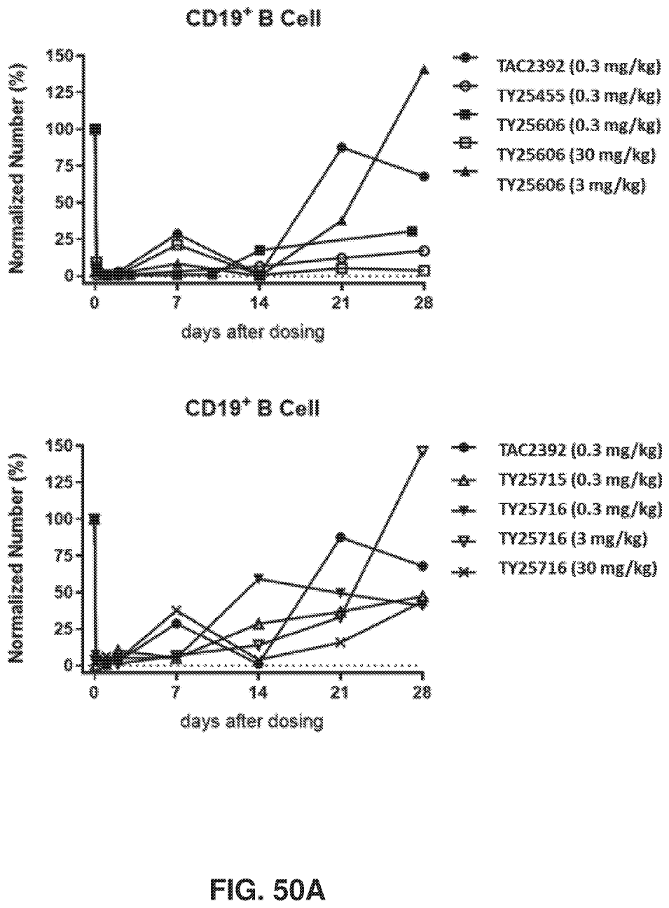

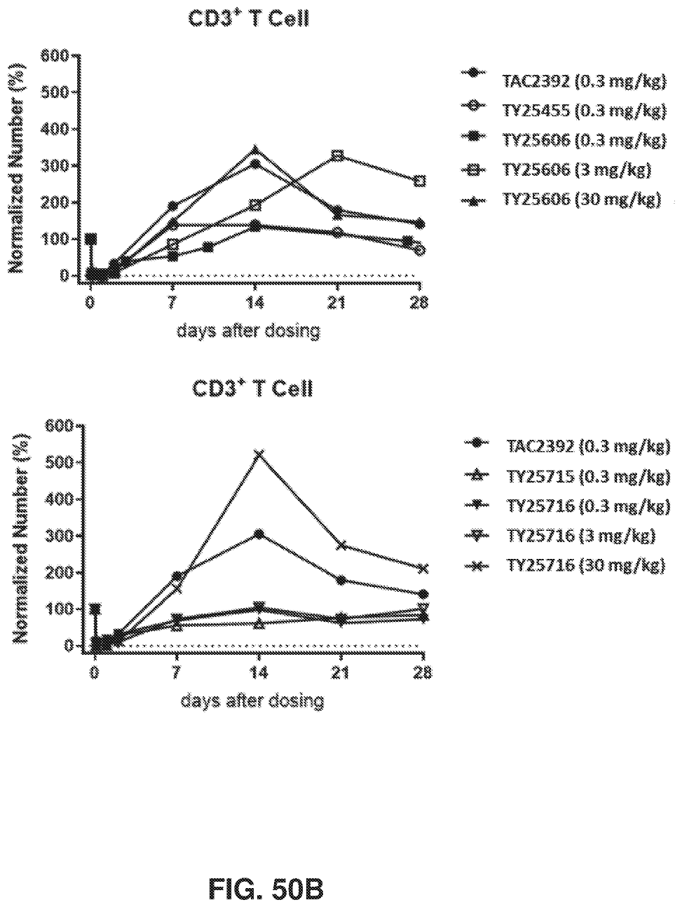

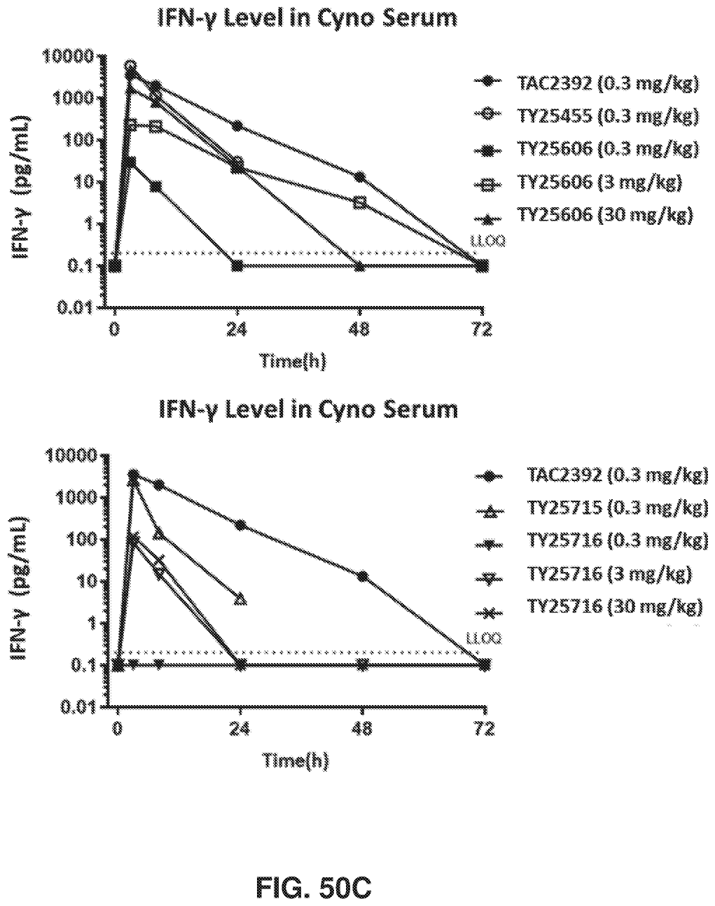

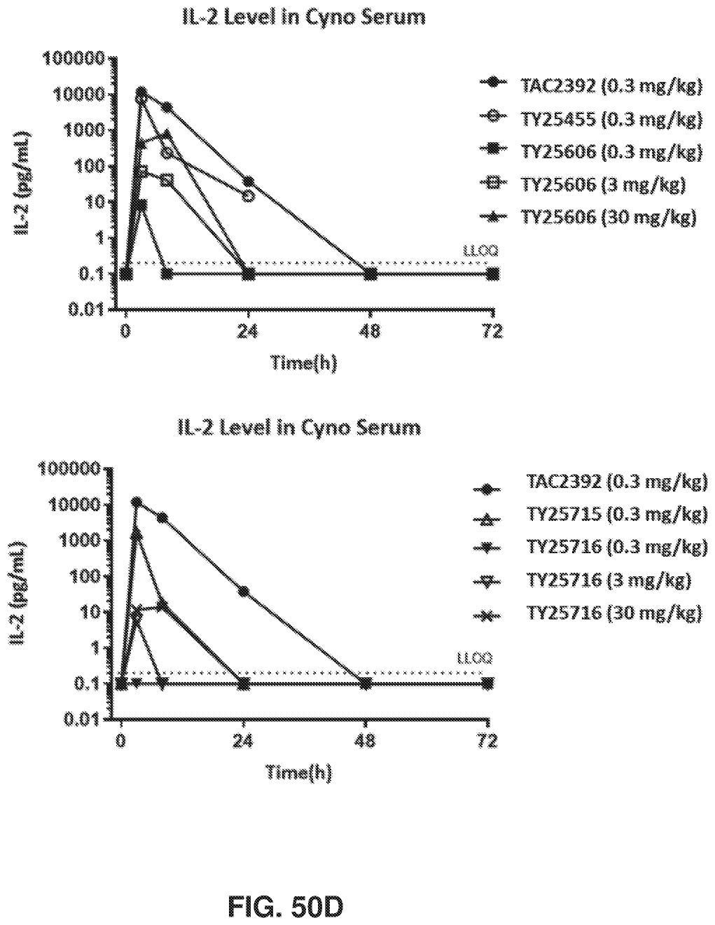

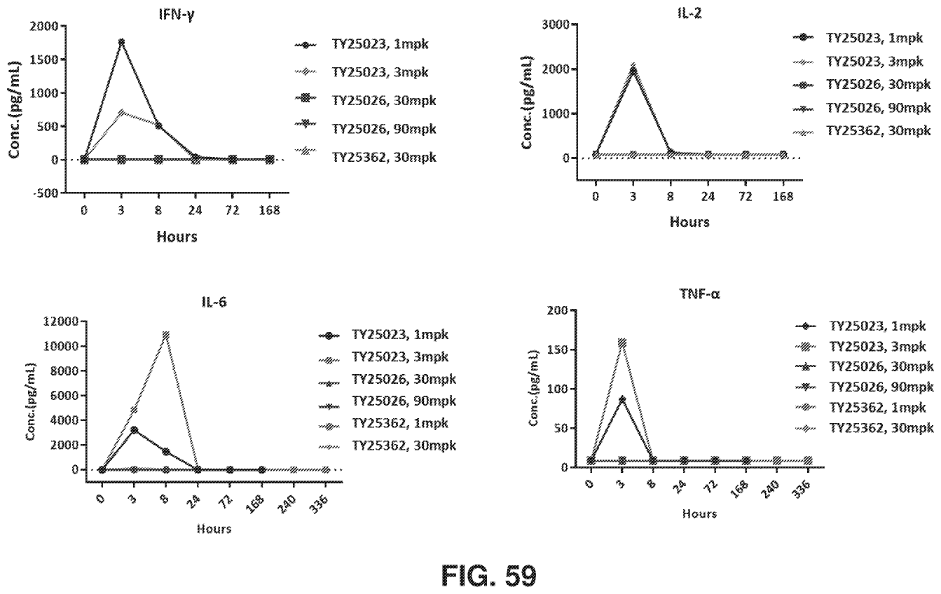

In some embodiments, the present application provides activatable multispecific antibodies (also referred to as “activatable multispecific T-cell engager” or “SAFEbody multispecific T-cell engager”) comprising a first antigen-binding fragment that specifically binds CD3 with weak affinity and a second antigen-binding fragment that specifically binds a target antigen, wherein the first antigen-binding fragment is fused to a first masking moiety via a first cleavable moiety. In some embodiments, the second antigen-binding fragment is fused to a second masking moiety via a second cleavable moiety. An exemplary type of activatable multispecific antibodies is a TAAxCD3 SAFEbody bispecific T-cell engager (“SAFE-bsAb”). A TAAxCD3 SAFE-bsAb molecule comprises an antigen-binding fragment of an antibody that specifically binds to a tumor-associate antigen (“TAA”), which may be masked or unmasked, and a masked anti-CD3 antigen-binding fragment. Exemplary SAFE-bsAbs described herein include HER2xCD3 SAFEbody (e.g., see Examples 1-2, 5-8 and 13) and CD20xCD3 SAFEbody (e.g., see Example 9-12). In circulation or healthy tissues, the activatable antibody is inactive because the masking moieties can block antigen binding. However, upon cleavage of the cleavable moieties at a target site (e.g., a disease site), the activatable antibody is activated to bind to both CD3 and the target antigen (e.g., TAA). Due to the weak affinity of the first antigen-binding fragment and the high masking efficiency of the first masking moiety, the activatable multispecific antibodies described herein provide a wide therapeutic window and reduce side effects associated with non-specific binding. For example, the exemplary TAAxCD3 SAFE-bsAbs in their activated forms have been observed to potently stimulate T-cell activation and TAA+ tumor cell killing. Additionally, no visible cytokine release syndrome and other adverse events were observed in exploratory toxicity studies of TAAxCD3 SAFE-bsAbs in cynomolgus monkeys, even at high dosage levels (see, for example, FIGS. 50C-50D and 59). Further, the activatable multispecific antibodies described herein exhibit improved stability and more robust expression levels relative to parental antibodies. The activatable multispecific antibodies described herein provide a safe and effective therapeutic approach for treatment of various diseases and conditions, including liquid and solid cancer that is associated with the target antigen.

Accordingly, one aspect of the present application provides an activatable multispecific antibody comprising: a) a first antigen-binding fragment that specifically binds CD3, wherein the first antigen-binding fragment is fused to a first masking moiety (MM1) via a first cleavable moiety (CM1); and b) a second antigen-binding fragment that specifically binds a target antigen; wherein the CM1 comprises a first cleavage site; wherein the MM1 inhibits binding of the activatable antibody to CD3 when the CM1 is not cleaved; wherein the activatable multispecific antibody binds to CD3 via the first antigen-binding fragment when the CM1 is cleaved; and wherein the first antigen-binding fragment binds CD3 with half-maximal binding at a concentration of antibody (EC₅₀) that is at least 10 nM (e.g., at least 100 nM) as determined by an enzyme-linked immunosorbent assay (ELISA, such as the ELISA assay of Example 3). In some embodiments, the MM1 comprises an amino acid sequence of SEQ ID NO: 35 or 417. In some embodiments, the CM1 comprises an amino acid sequence of SEQ ID NO: 77 or 418. In some embodiments, the target antigen is HER2. In some embodiments, the target antigen is CD20.

Also provided are isolated anti-CD3 antibodies, masked anti-CD3 antibodies (including activatable anti-CD3 antibodies), masked anti-HER2 antibodies (including activatable anti-HER2 antibodies), compositions, methods of preparation and methods of use.

### I. Definitions

Terms are used herein as generally used in the art, unless otherwise defined as follows.

The term “antibody” herein is used in the broadest sense and encompasses various antibody structures, including but not limited to (including full length monoclonal antibodies), multispecific antibodies (e.g., bispecific antibodies), and antibody fragments so long as they exhibit the desired biological activity.

The term “antigen-binding fragment” refers to one or more portions of an antibody that retain the ability to bind to the antigen of the antibody. Examples of “antigen-binding fragment” of an antibody include, but are not limited to, (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fv fragment consisting of the V_{L} and V_{H} domains of a single arm of an antibody, (v) a single chain Fv fragment comprising the VH and VL domains of an antibody, and the VH and VL domains are fused to each other; and (vi) a single chain Fab fragment comprising a single polypeptide comprising the V_{L}, V_{H}, C_{L} and C_{H1} domains.

The term “antibody” includes, but is not limited to, fragments that are capable of binding antigen, such as Fv, Fab, Fab′, and (Fab′)₂. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “hypervariable region” or “HVR,” as used herein, refers to each of the regions of an antibody variable domain, which are hypervariable in sequence. HVRs may form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), CDRs being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991)) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008)). Unless otherwise indicated, HVR residues and other residues in the variable domain (e.g., FR residues) are numbered herein according to Kabat et al., supra.

Table I below provides exemplary CDR definitions according to various algorithms known in the art.

TABLE I
CDR DEFINITIONS

| | Kabat¹ | Chothia² | MacCallum³ | IMGT⁴ | AHo⁵ |
|---|---|---|---|---|---|
| VH CDR1 | 31-35 | 26-32 | 30-35 | 27-38 | 25-40 |
| VH CDR2 | 50-65 | 53-55 | 47-58 | 56-65 | 58-77 |
| VH CDR3 | 95-102 | 96-101 | 93-101 | 105-117 | 109-137 |
| VL CDR1 | 24-34 | 26-32 | 30-36 | 27-38 | 25-40 |
| VL CDR2 | 50-56 | 50-52 | 46-55 | 56-65 | 58-77 |
| VL CDR3 | 89-97 | 91-96 | 89-96 | 105-117 | 109-137 |

¹Residue numbering follows the nomenclature of Kabat et al., J. Biol. Chem. 252: 6609-6616 (1977); Kabat et al., U.S. Dept. of Health and Human Services, “Sequences of proteins of immunological interest” (1991).
²Residue numbering follows the nomenclature of Chothia et al., J. Mol. Biol. 196: 901-917 (1987); Al-Lazikani B. et al., J. Mol. Biol., 273: 927-948 (1997).
³Residue numbering follows the nomenclature of MacCallum et al., J. Mol. Biol. 262: 732-745 (1996); Abhinandan and Martin, Mol. Immunol., 45: 3832-3839 (2008).
⁴Residue numbering follows the nomenclature of Lefranc M. P. et al., Dev. Comp. Immunol., 27: 55-77 (2003); and Honegger and Plückthun, J. Mol. Biol., 309: 657-670 (2001).
⁵Residue numbering follows the nomenclature of Honegger and Plückthun, J. Mol. Biol., 309: 657-670 (2001).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four framework regions (FRs) and three hypervariable regions (HVRs), arranged from amino-terminus to carboxy-terminus in the following order: FR1, HVR1, FR2, HVR2, FR3, HVR3, FR4. (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “EU numbering” or “amino acid position numbering based on EU numbering,” and variations thereof, refers to the numbering system used for heavy chain constant domains of the compilation of antibodies in Edelman, G. M. et al., Proc. Natl. Acad. USA, 63, 78-85 (1969). The EU numbering of residues may be determined for a given antibody by alignment at regions of homology of the sequence of the antibody with a “standard” EU numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

With respect to a heterodimeric protein (e.g., an activatable multispecific antibody) having two CH3 domains, a given amino acid position of a first CH3 domain is referred to as X, and the corresponding amino acid position of the second CH3 domain is referred to as X′. For example, N390C-S400′C refers to a heterodimeric protein (e.g., an activatable multispecific antibody) having a first CH3 domain having a N390C mutation and a second CH3 domain having a S400C mutation. All mutations or substitutions in the heterodimeric proteins (e.g., an activatable multispecific antibody) described herein are referred herein with respect to a wildtype, naturally occurring CH3 domain.

Unless indicated otherwise, all formula of polypeptide chains described herein list the components of the polypeptide in the order from the N-terminus to the C-terminus. For example, the formula VH2-CH1-hinge-CH2-first CH3 indicates that the polypeptide comprises, from the N-terminus to the C-terminus, the following structural components: VH2, CH1, hinge, CH2, and first CH3.

The term “heavy chain constant region” as used herein refers to a region comprising at least three heavy chain constant domains, CH1, CH2, and CH3, and a hinge region between CH1 and CH2. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a constant region is an IgM antibody, and an antibody comprising an F constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “CH2 domain” of a human IgG Fc region usually extends from about residues 231 to about 340 of the IgG according to the EU numbering system. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985).

The term “CH3 domain” comprises the stretch of residues C-terminal to a CH2 domain in an Fc region (i.e., from about amino acid residue 341 to about amino acid residue 447 of an IgG according to the EU numbering system).

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain constant region” as used herein refers to a region comprising a light chain constant domain, CL. Non-limiting exemplary light chain constant regions include λ and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

“Affinity” refers to the strength of the sum total of noncovalent interactions between a binding site of a molecule (e.g., an antibody) and its binding partner (e.g., an antigen). The affinity of a molecule X for its partner Y can generally be represented by the dissociation constant (K_{d}). Affinity can be measured by common methods known in the art, including those described herein. In the context of a multispecific antibody (e.g., a bispecific or trispecific antibody), affinity of the antibody with each binding specificity (i.e. target) can be measured.

The term “binds”, “specifically binds” or is “specific for” refers to measurable and reproducible interactions such as binding between a target and an antibody, which is determinative of the presence of the target in the presence of a heterogeneous population of molecules including biological molecules. For example, an antibody that binds or specifically binds a target (which can be an epitope) is an antibody that binds this target with greater affinity, avidity, more readily, and/or with greater duration than it binds other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (Kd) of ≤1 μM, ≤100 nM, ≤10 nM, ≤1 nM, or ≤0.1 nM. In some embodiments, an antibody specifically binds an epitope on a protein that is conserved among the protein from different species. In some embodiments, specific binding can include, but does not require exclusive binding.

The term “multispecific” as used in conjunction with an antibody refers to an antibody having polyepitopic specificity (i.e., is capable of specifically binding to two, three, or more, different epitopes on one biological molecule or is capable of specifically binding to epitopes on two, three, or more, different biological molecules).

An “affinity matured” antibody refers to an antibody with one or more alterations in one or more hypervariable regions (HVRs) compared to a parent antibody, which does not possess such alterations, such alterations resulting in an improvement in the affinity of the antibody for antigen. In some examples, an affinity-matured antibody refers to an antibody with one or more alterations in one or more complementarity determining regions (CDRs) compared to a parent antibody, which does not possess such alterations, such alterations resulting in an improvement in the affinity of the antibody for antigen.

A “chimeric antibody” as used herein refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, a (Fab′)₂, etc.

An “HVR-grafted antibody” as used herein refers to a humanized antibody in which one or more hypervariable regions (HVRs) of a first (non-human) species have been grafted onto the framework regions (FRs) of a second (human) species. In some examples, a “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XENOMOUSE®, and antibodies selected using in vitro methods, such as phage display, wherein the antibody repertoire is based on a human immunoglobulin sequence.

“Antibody-dependent cell-mediated cytotoxicity” or “ADCC” refers to a form of cytotoxicity in which secreted Ig bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, e.g., in U.S. Pat. Nos. 7,923,538, and 7,994,290.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1, U.S. Pat. Nos. 7,923,538, 7,994,290 and WO 1999/51642. See also, e.g., Idusogie et al., J. Immunol. 164: 4178-4184 (2000).

A polypeptide variant with “altered” FcR binding affinity or ADCC activity is one which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. Such variants which display decreased binding to an FcR may possess little or no appreciable binding to an FcR, e.g., 0-20% binding to the FcR compared to a native sequence IgG Fc region.

The terms “nucleic acid molecule”, “nucleic acid” and “polynucleotide” may be used interchangeably, and refer to a polymer of nucleotides. Such polymers of nucleotides may contain natural and/or unnatural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “peptide” are used interchangeably to refer to a polymer of amino acid residues, and are not limited to a minimum length. Such polymers of amino acid residues may contain natural or unnatural amino acid residues. Both full-length proteins and fragments thereof are encompassed by the definition. The terms also include post-expression modifications of the polypeptide, for example, glycosylation, sialylation, acetylation, phosphorylation, and the like. Furthermore, a “polypeptide” includes modifications, such as deletions, additions, and substitutions (generally conservative in nature), to the native sequence, as long as the polypeptide maintains the desired activity. These modifications may be deliberate, as through site-directed mutagenesis, or may be accidental, such as through mutations of hosts, which produce the proteins or errors due to PCR amplification.

A polypeptide “variant” means a biologically active polypeptide having at least 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least 80% amino acid sequence identity. In some embodiments, a variant will have at least 90% amino acid sequence identity. In some embodiments, a variant will have at least 95% amino acid sequence identity with the native sequence polypeptide.

As used herein, “Percent (%) amino acid sequence identity” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table A. Amino acid substitutions may be introduced into an antibody of interest and the products screened for a desired activity, e.g., retained/improved antigen binding, decreased immunogenicity, improved or reduced ADCC or CDC, or reduced crosslinking effects.

TABLE A
Exemplary Amino Acid Substitutions.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.
   Non-conservative substitutions will entail exchanging a member of one of these classes for another class.

The term “vector” is used to describe a polynucleotide that may be engineered to contain a cloned polynucleotide or polynucleotides that may be propagated in a host cell. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that may be used in colorimetric assays, e.g., (3-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Non-limiting exemplary mammalian cells include, but are not limited to, NSO cells, PER.C6® cells (Crucell), and 293 and CHO cells, and their derivatives, such as 293-6E and DG44 cells, respectively. The term “cell” includes the primary subject cell and its progeny.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated”.

The terms “individual” or “subject” are used interchangeably herein to refer to a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the invention contemplate any one or more of these aspects of treatment.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of cancer means to defer, hinder, slow, retard, stabilize, and/or postpone development of the disease. This delay can be of varying lengths of time, depending on the history of the disease and/or individual being treated. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The term “effective amount” used herein refers to an amount of an agent or a combination of agents, sufficient to treat a specified disorder, condition or disease such as to ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other undesired cell proliferation. In some embodiments, an effective amount is an amount sufficient to delay disease development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a”, “an”, and “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Antibodies

Certain aspects of the present application relate to multispecific antibodies, masked antibodies such as activatable antibodies (including activatable multispecific antibodies such as activatable bispecific T cell engager molecules), antigen-binding fragments thereof, or derivatives of such antibodies.

One aspect of the present application provides multispecific antibodies that are capable of binding to both T cells and target cells such as tumor cells. In some embodiments, the multispecific antibody is bispecific. In some embodiments, the multispecific antibody is trispecific. In some embodiments, the multispecific antibody binds to CD3 on the surface of T cells. Because of their on-target off-tumor effects, traditional BiTE molecules are associated with high cytotoxicity, including toxicity to the central nervous system (CNS) and cytokine storms. Therefore, there is a need for antibodies capable of binding a T cell and a target cell such as a tumor cell with enhanced specificity and reduced side effects.

In some embodiments, there is provided a multispecific antibody comprising: a) a first antigen-binding fragment that specifically binds CD3, wherein the first antigen-binding fragment is fused to a first masking moiety (MM1); and b) a second antigen-binding fragment that specifically binds a target antigen (e.g., a tumor antigen, such as HER2, CD20, TROP2, BCMA, or CD19); wherein the MM1 competes with CD3 to specifically bind the CD3-binding moiety; wherein the multispecific antibody binds to CD3 via the first antigen-binding fragment; and wherein the first antigen-binding fragment binds CD3 with half-maximal binding at a concentration of antibody (EC₅₀) that is at least 10 nM (e.g., at least 100 nM) as determined by an ELISA assay (e.g., as described in Example 5). In some embodiments, the first antigen-binding fragment binds CD3 with a dissociation constant (Kd) of at least 50 nM.

In some embodiments, there is provided a multispecific antibody comprising: a) a first antigen-binding fragment that specifically binds CD3, wherein the first antigen-binding fragment is fused to a first masking moiety (MM1); and b) a second antigen-binding fragment that specifically binds a target antigen (e.g., a tumor antigen, such as HER2, CD20, TROP2, BCMA, or CD19); wherein the MM1 competes with CD3 to specifically bind the CD3-binding moiety; wherein the multispecific antibody binds to CD3 via the first antigen-binding fragment; wherein the first antigen-binding fragment binds CD3 with half-maximal binding at a concentration of antibody (EC₅₀) that is at least 10 nM (e.g., at least 100 nM) as determined by an ELISA assay (e.g., as described in Example 5), and wherein the MM1 has a masking efficiency of at least 50 as determined by a Jurkat NFAT reporter assay (e.g., the assay in Example 3).

### A. Activatable Multispecific T Cell Engagers

One aspect of the present application provides activatable multispecific antibodies that are capable of binding to both T cells and target cells such as tumor cells. In some embodiments, the activatable antibody is bispecific. In some embodiments, the activatable antibody is trispecific. For example, in some embodiments, the activatable multispecific antibodies are activatable bispecific T cell engagers (“BiTE”). In some embodiments, the activatable multispecific antibody binds to CD3 on the surface of T cells. Because of their on-target off-tumor effects, traditional BiTE molecules are associated with high cytotoxicity, including toxicity to the central nervous system (CNS) and cytokine storms. Therefore, there is a need for activatable BiTE molecules with enhanced specificity and reduced side effects.

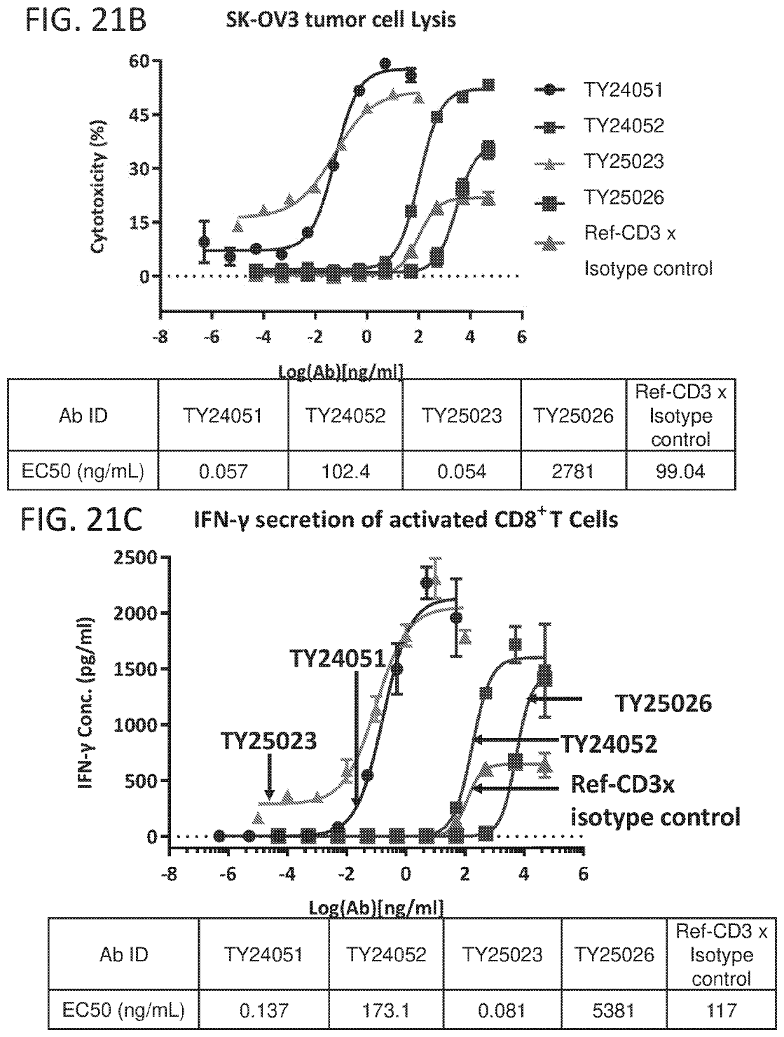

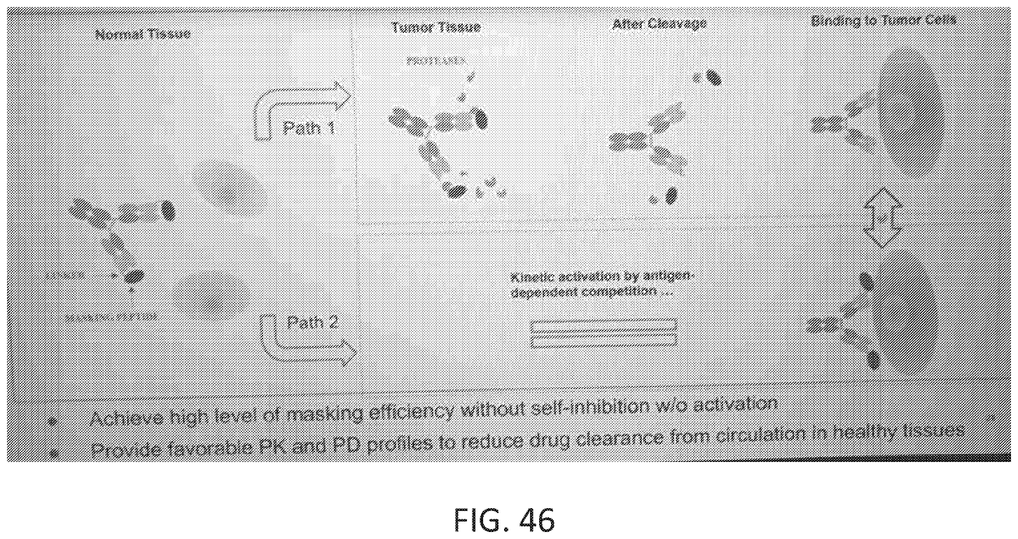

The present disclosure is based in part on the discovery of anti-CD3 BiTE molecules that bind CD3 with a relatively weak binding affinity (see, for example, FIGS. 21A-21C and Table 6), as well as masking moieties that reduce binding of the anti-CD3 antibodies with high efficiency (see, for example, Tables 4-5). FIG. 46 illustrates potential mechanisms of action of activatable BiTE molecules. Without wishing to be bound by theory, it is believed that activatable BiTE molecules with relatively weak affinities for CD3 and/or high masking efficiency for blocking CD3 binding have less severe side effects than traditional BiTE molecules. Due to this reduction in the severity of side effects, it is believed that the activatable BiTE molecules described herein allow for a greater therapeutic window. That is, activatable BiTE molecules described herein may be administered to treat disease effectively without producing toxic effects such as cytokine storm commonly associated with traditional BiTE molecules, e.g., BiTE molecules having stronger CD3 binding affinities. Accordingly, the present application provides antibodies or antigen-binding fragments thereof, activatable antibodies, activatable multispecific antibodies, activatable antibody fragments, and polypeptides that bind specifically to human CD3 with a relatively weak binding affinity.

In some embodiments, there is provided an activatable multispecific antibody comprising: a) a first antigen-binding fragment that specifically binds CD3, wherein the first antigen-binding fragment is fused to a first masking moiety (MM1) via a first cleavable moiety (CM1); and b) a second antigen-binding fragment that specifically binds a target antigen (e.g., a tumor antigen, such as HER2, CD20, TROP2, BCMA, or CD19); wherein the CM1 comprises a first cleavage site; wherein the MM1 inhibits binding of the activatable antibody to CD3 when the CM1 is not cleaved; wherein the activatable multispecific antibody binds to CD3 via the first antigen-binding fragment when the CM1 is cleaved; and wherein the first antigen-binding fragment binds CD3 with half-maximal binding at a concentration of antibody (EC₅₀) that is at least 10 nM (e.g., at least 100 nM) as determined by an ELISA assay (e.g., as described in Example 5). In some embodiments, the first antigen-binding fragment binds CD3 with a dissociation constant (Kd) of at least 50 nM.

In some embodiments, there is provided an activatable multispecific antibody comprising: a) a first antigen-binding fragment that specifically binds CD3, wherein the first antigen-binding fragment is fused to a first masking moiety (MM1) via a first cleavable moiety (CM1); and b) a second antigen-binding fragment that specifically binds a target antigen (e.g., a tumor antigen, such as HER2, CD20, TROP2, BCMA, or CD19); wherein the CM1 comprises a first cleavage site; wherein the MM1 inhibits binding of the activatable antibody to CD3 when the CM1 is not cleaved; wherein the activatable multispecific antibody binds to CD3 via the first antigen-binding fragment when the CM1 is cleaved; wherein the first antigen-binding fragment binds CD3 with half-maximal binding at a concentration of antibody (EC₅₀) that is at least 10 nM (e.g., at least 100 nM) as determined by an ELISA assay (e.g., as described in Example 5), and wherein the MM1 has a masking efficiency of at least 50 as determined by a Jurkat NFAT reporter assay (e.g., the assay in Example 3).

In some embodiments, there is provided an activatable multispecific antibody comprising: a) a first antigen-binding fragment comprising a VH1 and a VL1 of an anti-CD3 antibody that specifically binds CD3, wherein the first antigen-binding fragment is fused to a first masking moiety (MM1) via a first cleavable moiety (CM1); and b) a second antigen-binding fragment comprising a VH2 and a VL2 of an antibody that specifically binds a target antigen (e.g., a tumor antigen, such as HER2, CD20, TROP2, BCMA, or CD19); wherein the MM1 is fused to the N-terminus of the VL1 via the CM1, wherein the CM1 comprises a first cleavage site; wherein the MM1 inhibits binding of the activatable antibody to CD3 when the CM1 is not cleaved; wherein the activatable multispecific antibody binds to CD3 via the first antigen-binding fragment when the CM1 is cleaved; and wherein the first antigen-binding fragment binds CD3 with half-maximal binding at a concentration of antibody (EC₅₀) that is at least 10 nM (e.g., at least 100 nM) as determined by an ELISA assay (e.g., as described in Example 5). In some embodiments, the MM1 has a masking efficiency of at least 50 as determined by a Jurkat NFAT reporter assay (e.g., the assay in Example 3). In some embodiments, the first antigen-binding fragment is selected from the group consisting of a Fab, a Fv, a scFab and a scFv. In some embodiments, the first antigen-binding fragment is a scFv comprising, from N-terminus to C-terminus, VL1, an optional linker, and VH1.

In some embodiments, there is provided an activatable multispecific antibody comprising a first polypeptide, a second polypeptide, and a third polypeptide, wherein:
(i) the first polypeptide comprises a structure represented by the formula:

VH2-CH1-hinge-CH2-first CH3 (1a);

(ii) the second polypeptide comprises a structure represented by the formula:

MM1-CM1-VL1-VH1-hinge-CH2-second CH3 (1b);

and
(iii) the third polypeptide comprises a structure represented by the formula:

VL2-CL (1c);

wherein:
   VL1 is a first immunoglobulin light chain variable domain;
   VH1 is a first immunoglobulin heavy chain variable domain;
   VL2 is a second immunoglobulin light chain variable domain;
   VH2 is a second immunoglobulin heavy chain variable domain;
   CL is an immunoglobulin light chain constant domain;
   CH1 is an immunoglobulin heavy chain constant domain 1;
   CH2 is an immunoglobulin heavy chain constant domain 2;
   first CH3 is a first immunoglobulin heavy chain constant domain 3;
   second CH3 is a second immunoglobulin heavy chain constant domain 3;
   hinge is an immunoglobulin hinge region connecting the CH1 and CH2 domains;
   MM1 is a masking moiety; and
   CM1 is a cleavable moiety comprising a cleavage site;
      wherein VH1 and VL1 associate to form a scFv that specifically binds CD3 with half-maximal binding at a concentration of antibody (EC₅₀) that is at least 10 nM (e.g., at least 100 nM) as determined by an ELISA assay (e.g., as described in Example 5), wherein VH2 and VL2 associate to form a Fv that specifically binds a target antigen (e.g., a tumor antigen, such as HER2, CD20, TROP2, BCMA, or CD19), wherein the MM1 inhibits binding of the activatable antibody to CD3 when the CM1 is not cleaved; and wherein the activatable multispecific antibody binds to CD3 via the first antigen-binding fragment when the CM1 is cleaved. In some embodiments, the MM1 has a masking efficiency of at least 50 as determined by a Jurkat NFAT reporter assay (e.g., the assay in Example 3). In some embodiments, the multispecific antibody (e.g., the second polypeptide thereof) comprises an amino acid linker between VL1 and VH1.

In some embodiments, there is provided an activatable multispecific antibody comprising a first polypeptide, a second polypeptide, a third polypeptide, and a fourth polypeptide, wherein:
(i) the first polypeptide comprises a structure represented by the formula:

VH1-CH1-hinge-CH2-first CH3 (3a);

(ii) the second polypeptide comprises a structure represented by the formula:

VH2-CH1-hinge-CH2-second CH3 (3b);

(iii) the third polypeptide comprises a structure represented by the formula:

MM1-CL1-VL1-CL (3c);

and
(iv) the fourth polypeptide comprises a structure represented by the formula:

VL2-CL (3d);

wherein:
VL1 is a first immunoglobulin light chain variable domain;
VH1 is a first immunoglobulin heavy chain variable domain;
VL2 is a second immunoglobulin light chain variable domain;
VH2 is a second immunoglobulin heavy chain variable domain;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a masking moiety; and
CM1 is a cleavable moiety comprising a cleavage site;
   wherein VH1 and VL1 associate to form a first Fv that specifically binds CD3 with half-maximal binding at a concentration of antibody (EC₅₀) that is at least 10 nM (e.g., at least 100 nM) as determined by an ELISA assay (e.g., as described in Example 5); wherein VL2 and VH2 associate to form a second Fv that specifically binds a target antigen (e.g., a tumor antigen, such as HER2, CD20, TROP2, BCMA, or CD19), wherein the MM1 inhibits binding of the activatable antibody to CD3 when the CM1 is not cleaved; and wherein the activatable multispecific antibody binds to CD3 via the first antigen-binding fragment when the CM1 is cleaved. In some embodiments, the MM1 has a masking efficiency of at least 50 as determined by a Jurkat NFAT reporter assay (e.g., the assay in Example 3).

In some embodiments, there is provided an activatable multispecific antibody comprising: a) a first antigen-binding fragment that specifically binds CD3, wherein the first antigen-binding fragment is fused to a first masking moiety (MM1) via a first cleavable moiety (CM1); and b) a second antigen-binding fragment that specifically binds a target antigen (e.g., a tumor antigen, such as HER2, CD20, TROP2, BCMA, or CD19), wherein the second antigen-binding fragment is fused to a second masking moiety (MM2) via a second cleavable moiety (CM2); wherein the CM1 comprises a first cleavage site; wherein the MM1 inhibits binding of the activatable antibody to CD3 when the CM1 is not cleaved; wherein the activatable multispecific antibody binds to CD3 via the first antigen-binding fragment when the CM1 is cleaved; wherein the CM2 comprises a second cleavage site; wherein the MM2 inhibits binding of the activatable antibody to the target antigen when the CM2 is not cleaved; wherein the activatable multispecific antibody binds the target antigen via the second antigen-binding fragment when the CM2 is cleaved; and wherein the first antigen-binding fragment binds CD3 with half-maximal binding at a concentration of antibody (EC₅₀) that is at least 10 nM (e.g., at least 100 nM) as determined by an ELISA assay (e.g., as described in Example 5). In some embodiments, the MM1 has a masking efficiency of at least 50 as determined by a Jurkat NFAT reporter assay (e.g., the assay in Example 3).

In some embodiments, there is provided an activatable multispecific antibody comprises a first polypeptide, a second polypeptide, and a third polypeptide, wherein:
(i) the first polypeptide comprises a structure represented by the formula:

VH2-CH1-hinge-CH2-first CH3 (2a);

(ii) the second polypeptide comprises a structure represented by the formula:

MM1-CM1-VL1-VH1-hinge-CH2-second CH3 (2b);

and
(iii) the third polypeptide comprises a structure represented by the formula:

MM2-CM2-VL2-CL (2c);

wherein:
   VL1 is a first immunoglobulin light chain variable domain;
   VH1 is a first immunoglobulin heavy chain variable domain;
   VL2 is a second immunoglobulin light chain variable domain;
   VH2 is a second immunoglobulin heavy chain variable domain;
   CL is an immunoglobulin light chain constant domain;
   CH1 is an immunoglobulin heavy chain constant domain 1;
   CH2 is an immunoglobulin heavy chain constant domain 2;
   first CH3 is a first immunoglobulin heavy chain constant domain 3;
   second CH3 is a second immunoglobulin heavy chain constant domain 3;
   hinge is an immunoglobulin hinge region connecting the CH1 and CH2 domains;
   MM1 is a first masking moiety;
   CM1 is a first cleavable moiety comprising a first cleavage site;
   MM2 is a second masking moiety;
   CM2 is a second cleavable moiety comprising a second cleavage site;
      wherein VH1 and VL1 associate to form a scFv that specifically binds CD3 with half-maximal binding at a concentration of antibody (EC₅₀) that is at least 10 nM (e.g., at least 100 nM) as determined by an ELISA assay (e.g., as described in Example 5), wherein VH2 and VL2 associate to form a Fv that specifically binds a target antigen (e.g., a tumor antigen, such as HER2, CD20, TROP2, BCMA, or CD19), wherein the MM1 inhibits binding of the activatable antibody to CD3 when the CM1 is not cleaved; wherein the activatable multispecific antibody binds to CD3 via the first antigen-binding fragment when the CM1 is cleaved; wherein the MM2 inhibits binding of the activatable antibody to the target antigen when the CM2 is not cleaved; and wherein the activatable multispecific antibody binds the target antigen via the second antigen-binding fragment when the CM2 is cleaved. In some embodiments, the MM1 has a masking efficiency of at least 50 as determined by a Jurkat NFAT reporter assay (e.g., the assay in Example 3). In some embodiments, the multispecific antibody (e.g., the second polypeptide thereof) comprises an amino acid linker between VL1 and VH1.

In some embodiments, there is provided an activatable multispecific antibody comprising a first polypeptide, a second polypeptide, a third polypeptide, and a fourth polypeptide, wherein:
(i) the first polypeptide comprises a structure represented by the formula:

VH1-CH1-hinge-CH2-first CH3 (4a);

(ii) the second polypeptide comprises a structure represented by the formula:

VH2-CH1-hinge-CH2-second CH3 (4b);

(iii) the third polypeptide comprises a structure represented by the formula:

MM1-CL1-VL1-CL (4c);

and
(iv) the fourth polypeptide comprises a structure represented by the formula:

MM2-CL2-VL2-CL (4d);

wherein:
VL1 is a first immunoglobulin light chain variable domain;
VH1 is a first immunoglobulin heavy chain variable domain;
VL2 is a second immunoglobulin light chain variable domain;
VH2 is a second immunoglobulin heavy chain variable domain;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a first masking moiety;
CM1 is a first cleavable moiety comprising a first cleavage site;
MM2 is a second masking moiety;
CM2 is a second cleavable moiety comprising a second cleavage site;
   wherein VH1 and VL1 associate to form a first Fv that specifically binds CD3 with half-maximal binding at a concentration of antibody (EC₅₀) that is at least 10 nM (e.g., at least 100 nM) as determined by an ELISA assay (e.g., as described in Example 5); wherein VL2 and VH2 associate to form a second Fv that specifically binds a target antigen (e.g., a tumor antigen, such as HER2, CD20, TROP2, BCMA, or CD19), wherein the MM1 inhibits binding of the activatable antibody to CD3 when the CM1 is not cleaved; wherein the activatable multispecific antibody binds to CD3 via the first antigen-binding fragment when the CM1 is cleaved; wherein the MM2 inhibits binding of the activatable antibody to the target antigen when the CM2 is not cleaved; and wherein the activatable multispecific antibody binds the target antigen via the second antigen-binding fragment when the CM2 is cleaved. In some embodiments, the MM1 has a masking efficiency of at least 50 as determined by a Jurkat NFAT reporter assay (e.g., the assay in Example 3).

In some embodiments, the first antigen-binding fragment binds CD3 (e.g., human CD3) with weak binding affinity. In some embodiments, the first antigen-binding fragment binds CD3 with a relatively weak binding affinity relative to the K_{D} of a reference antibody for CD3. In some embodiments, the first antigen-binding fragment binds CD3 with a higher dissociation constant than the reference antibody for CD3. In some embodiments, the first antigen-binding fragment binds CD3 with a lower association constant than the reference antibody for CD3. In some embodiments, the reference antibody is SP34. In some embodiments, the binding affinity of the first antigen-binding fragment to CD3 is measured when the first antigen-binding fragment is present as an isolated antigen-binding fragment or as part of a monospecific antibody. In some embodiments, the binding affinity of the first antigen-binding fragment to CD3 is measured when the first antigen-binding fragment is present in a multispecific antibody, or in an activated form of the activatable multispecific antibody, i.e., with CM1 cleaved and MM1 unbound to the first antigen-binding fragment.

In some embodiments, the first antigen-binding fragment binds CD3 (e.g., human CD3) with half-maximal binding at a concentration of antibody (EC₅₀) that is at least about any one of 9, 10, 11, 12, 13, 14, 15, 20, 30, 40, 50, 60, 70, 75, 80, 85, 90, 95, 100, 101, 102, 103, 104, 105, 106, 107, 108, 109, 110, 111, 112, 113, 114, 115, 116, 117, 118, 119, 120, 125, 130, 135, 140, 145, 150, 160, 175, 200, 250 or more nM as determined by an enzyme-linked immunosorbent assay (ELISA), including any value or range in between these values. In some embodiments, the first antigen-binding domain binds human CD3 with an EC₅₀ of about any one of 10-50, 50-100, 100-150, 150-200, 10-100, 10-110, 9-111, 10-115, 75-150, 100-150, 10-150, 10-200, 50-125, 10-20, 20-50, 50-75, 75-125, 90-120, 100-120, 100-110, 110-120, 50-150, 50-200, or 10-250 nM, as determined by an enzyme-linked immunosorbent assay (ELISA). In some embodiments, the EC₅₀ is determined by an ELISA measuring binding of an unmasked multispecific antibody to CD3 (e.g., human CD3 or human CD3δε). In some embodiments, the first antigen binding fragment is a scFv, and the EC₅₀ is determined by an ELISA measuring binding of an unmasked multispecific antibody to CD3 (e.g., human CD3 or human CD3δε). In some embodiments, the EC₅₀ is determined by an ELISA measuring binding of a parental multispecific antibody that lacks a CM and an MM to CD3 (e.g., human CD3 or human CD3δε). In some embodiments, the first antigen binding fragment is a scFv, and the EC₅₀ is determined by an ELISA measuring binding of a parental multispecific antibody that lacks a CM and an MM to CD3 (e.g., human CD3 or human CD3δε). In some embodiments, the EC₅₀ is determined by an ELISA measuring binding of an antigen-binding fragment that binds CD3 (e.g., an isolated anti-CD3 scFv or scFv-Fc fusion protein) to CD3 (e.g., human CD3 or human CD3δε).

In some embodiments, the first antigen-binding fragment binds CD3 (e.g., human CD3) with an EC₅₀ that is at least about any one of 2, 3, 4, 5, 6, 7, 8, 9, 10, 15, 20, 25, 30, 35, 40, 45, 50, 55, 60, 70, 75, 80, 85, 90, 95, 100, 110, 120, 130, 140, 150, 200, 300, 400, 500 times or more higher than the EC₅₀ of a reference antibody (e.g., SP34), including any value or range in between these values. In some embodiments, the first antigen-binding fragment binds CD3 (e.g., human CD3) with an EC₅₀ that is about any one of 2-10, 10-20, 20-30, 30-40, 40-50, 50-60, 60-75, 75-100, 100-200, 200-500, 2-5, 5-10, 5-20, 5-30, 5-40, 5-50, 5-55, 5-60, 10-20, 10-30, 10-40, 10-50, 10-60, 20-40, 20-55, 30-60, 10-30, or 5-100 times the EC₅₀ of a reference antibody (e.g., SP34). In some embodiments, the EC₅₀ of the first antigen-binding fragment and the reference antibody are measured under the same experimental conditions. In some embodiments, the EC₅₀ of the first antigen-binding fragment and the reference antibody are measured in the same antibody format. In some embodiments, the EC₅₀ is determined by measuring binding of an unmasked multispecific antibody and an unmasked multispecific reference antibody to CD3 (e.g., human CD3 or human CD3δε). In some embodiments, the unmasked multispecific reference antibody comprises a CD3-binding moiety corresponding to SP34 (e.g., comprising the six CDRs of SP34). In some embodiments, the EC₅₀ is determined by measuring binding of a parental multispecific antibody that lacks a CM and an MM and a reference parental multispecific antibody that lacks a CM and an MM to CD3 (e.g., human CD3 or human CD3δε). In some embodiments, the reference parental multispecific antibody that lacks a CM and an MM comprises a CD3-binding moiety corresponding to SP34 (e.g., comprising the six CDRs of SP34). In some embodiments, the Kd of the first antigen-binding fragment binds CD3 and the EC₅₀ of the reference antibody are determined by an ELISA, such as the ELISA as described in Example 3. In some embodiments, the Kd of the first antigen-binding fragment binds CD3 and the EC₅₀ of the reference antibody are determined by a cell-based assay, such as a Jurkat NFAT reporter assay as described in Example 3.

In some embodiments, the first antigen-binding fragment binds CD3 (e.g., human CD3) with a relatively weak dissociation constant (Kd) compared to a reference antibody (e.g., SP34), such as at least about any one of2, 3, 4, 5, 6, 7, 8, 9, 10, 15, 20, 25, 30, 35, 40, 45, 50, 55, 60, 70, 75, 80, 85, 90, 95, 100, 110, 120, 130, 140, 150, 200, 300, 400, 500 times or more weaker than the Kd of the reference antibody, including any value or range in between these values. In some embodiments, the first antigen-binding fragment binds CD3 (e.g., human CD3) with a dissociation constant (Kd) that is about any one of 2-10, 10-20, 20-30, 30-40, 40-50, 50-60, 60-75, 75-100, 100-200, 200-500, 2-5, 5-10, 5-20, 5-30, 5-40, 5-50, 5-55, 5-60, 10-20, 10-30, 10-40, 10-50, 10-60, 20-40, 20-55, 30-60, 10-30, or 5-100 times weaker than the Kd of a reference antibody (e.g., SP34). In some embodiments, the Kd of the first antigen-binding fragment and the reference antibody are measured under the same experimental conditions. In some embodiments, the Kd of the first antigen-binding fragment and the reference antibody are measured in the same antibody format. In some embodiments, the Kd is determined by measuring binding of an unmasked multispecific antibody and an unmasked multispecific reference antibody to CD3 (e.g., human CD3 or human CD3δε). In some embodiments, the unmasked multispecific reference antibody comprises a CD3-binding moiety corresponding to SP34 (e.g., comprising the six CDRs of SP34). In some embodiments, the Kdis determined by measuring binding of a parental multispecific antibody that lacks a CM and an MM and a reference parental multispecific antibody that lacks a CM and an MM to CD3 (e.g., human CD3 or human CD3δε). In some embodiments, the reference parental multispecific antibody that lacks a CM and an MM comprises a CD3-binding moiety corresponding to SP34 (e.g., comprising the six CDRs of SP34). In some embodiments, the Kd of the first antigen-binding fragment binds CD3 and the Kd of the reference antibody are determined by an ELISA.

In some embodiments, the first antigen-binding fragment binds CD3 (e.g., human CD3) with a dissociation constant (Kd) of at least about any one of 10, 20, 30, 40, 50, 60, 70, 80, 90, 100, 125, 150, 175, 200, 250, 300, 400, 500 or more nM, including any value or range in between these values. In some embodiments, the first antigen-binding fragment binds CD3 (e.g., human CD3) with a dissociation constant (Kd) of at least about any one of 1, 10, or 100 PM, including any value or range in between these values (when in the activated form). In some embodiments, the first antigen-binding fragment binds CD3 (e.g., human CD3) with a dissociation constant (Kd) of about any one of 10-50, 50-100, 100-200, 200-500, 500-1000, 10-100, 100-500, 100-1000, 50-200, 50-250, 50-500 or 10-1000 nM.

In some embodiments, the first antigen-binding fragment binds CD3 (e.g., human CD3) with a relatively fast off-rate (k_{off}) compared to a reference antibody (e.g., SP34), such as at least about any one of 2, 5, 10, 20, 50, 100, 200 times or more faster than the k_{off} of the reference antibody, including any value or range in between these values.

In some embodiments, the first antigen-binding fragment binds CD3 (e.g., human CD3) with a relatively slow on-rate (kₒₙ) compared to a reference antibody (e.g., SP34), such as at least about any one of 2, 5, 10, 20, 50, 100, 200 times or more slower than the kₒₙ of the reference antibody, including any value or range in between these values.

In some embodiments, the first antigen-binding fragment binds CD3 (e.g., human CD3) with a relatively small dissociation constant (k_{d}) compared to a reference antibody (e.g., SP34), such as at least about any one of 2, 5, 10, 20, 50, 100, 200 times or more smaller than the k_{d} of the reference antibody, including any value or range in between these values.

In some embodiments, the first antigen-binding fragment binds CD3 (e.g., human CD3) with a relatively large association constant (kₐ) compared to a reference antibody (e.g., SP34), such as at least about any one of 2, 5, 10, 20, 50, 100, 200 times or more larger than the kₐ of the reference antibody.

Methods of measuring the ability of an antibody (e.g., an activatable multispecific antibody) to bind an antigen are known in the art, including, without limitation, via BIAcore analysis, surface plasmon resonance, ELISAs, flow cytometry, and cell-based assays (e.g., measuring binding to Jurkat cells) (See e.g., Example 5 and Table 6). The EC₅₀, dissociation constant (k_{d}), affinity constant (kₐ), off-rate (k_{off}), and/or on-rate (kₒₙ) of binding to CD3 (e.g., human CD3) may be measured in various contexts. In some embodiments, binding to CD3 (e.g., human CD3) is measured using an antigen-binding fragment that binds CD3 (e.g., a scFv or scFv-Fc fusion protein). In some embodiments, binding to CD3 (e.g., human CD3) is measured using an unmasked multispecific antibody. In some embodiments, binding to CD3 (e.g., human CD3) is measured using an activatable antibody (e.g., activatable multispecific antibody) wherein the cleavable moiety associated with the anti-CD3 antigen-binding fragment is cleaved. In some embodiments, binding to human CD3δε is measured. In some embodiments, binding to human CD3δε fused with an Fc fragment is measured. In some embodiments, binding to Jurkat cells is measured.

In some embodiments, an ELISA is performed using human CD3 (ε and δ chain heterodimer) fused with human Fc fragment as a binding substrate. An exemplary ELISA method is as follows:
1. 2 μg/mL of human CD3 (ε and δ chain heterodimer) fused with human Fc fragment is prepared and used to coat AN ELISA plate at 2-8° C. overnight.
2. After washing and blocking, 50 μL serial diluted IgG (e.g., a first antigen-binding fragment, an unmasked multispecific antibody, or an activatable antibody (e.g., activatable multispecific antibody) wherein the cleavable moiety associated with the anti-CD3 antigen-binding fragment is cleaved) is added and incubated at 37° C. for 1 hour.
3. Plates are washed three times and then incubated with 50 μL/well TMB substrate at room temperature for about 20 minutes.
4. The reaction is stopped.
5. Absorbance at 450 nm is measured.
6. The concentration of each antibody that produced half-maximal binding to CD3εδ is determined as the EC₅₀ in nM.

The first antigen-binding fragment and/or the second antigen-binding fragment may be of any suitable format, including, but are not limited to, a Fab, a Fv, a scFab and a scFv. The antigen-binding fragment may have a single polypeptide chain, or two or more polypeptide chains. The masking moiety (e.g., MM1 or MM2) may be fused to the N-terminus of any one of the polypeptide chain of an antigen-binding fragment that has multiple polypeptide chains. In some embodiments, the masking moiety (e.g., MM1 or MM2) is fused to the N-terminus of a VL (e.g., VL1 or VL2) of the antigen-binding fragment. In some embodiments, the masking moiety (e.g., MM1 or MM2) is fused to the N-terminus of a VH (e.g., VH1 or VH2) of the antigen-binding fragment.

The first antigen-binding fragment may be derived from any one of the anti-CD3 antibodies described herein, which have an EC₅₀ of at least 10 nM (e.g., at least 100 nM) as determined by an ELISA assay (e.g., as described in Example 5). Any one of the anti-CD3 antibodies and antigen-binding fragments described in Section i) “Anti-CD3 antibody” and Tables 5B-5H may be used.

In some embodiments, the first antigen-binding fragment comprises one, two, three, four, five, or six CDRs of an antibody as shown in Table 7. In some embodiments, the first antigen-binding fragment of the multispecific antibody comprises one, two, three, four, five, or six CDRs of the anti-CD3 antibody TY24051, TY25236, TY25023, TY25024, TY25237, TY25228, TY25227, TY25230, TY25229, TY25238, TY25239, TY25243, TY25231, TY25244, TY25241, or TY25240, as shown in Table 7. In some embodiments, the first antigen-binding fragment comprises a VH1 and/or a VL1 as shown in Table 8. In some embodiments, the first antigen-binding fragment comprises a VH1 and/or a VL1 of the anti-CD3 antibody TY24051, TY25236, TY25023, TY25024, TY25237, TY25228, TY25227, TY25230, TY25229, TY25238, TY25239, TY25243, TY25231, TY25244, TY25241, or TY25240, as shown in Table 8.

In some embodiments, the first antigen-binding fragment comprises one, two, three, four, five, or six CDRs of antibody TY25023 as shown in Table 7. In some embodiments, the first antigen-binding fragment comprises a VH and/or a VL of antibody TY25023 as shown in Table 8. In some embodiments, the first antigen-binding fragment comprises a scFv of antibody TY25023 as shown in Table 9. In some embodiments, the first antigen-binding fragment comprises a heavy chain of antibody TY25023 as shown in Table 12.

In some embodiments, the first antigen-binding fragment comprises a VH1 sequence having at least 90%, 91%, 92%, 93%, 94%, 95%, 96%, 97%, 98%, 99%, or 100% sequence identity to the amino acid sequence of SEQ ID NO: 402. In certain embodiments, a VH1 sequence contains substitutions (e.g., conservative substitutions), insertions, or deletions relative to the amino acid sequence of SEQ ID NO:402, but retains the same ability to bind CD3 as the antibody comprising SEQ ID NO: 402. In certain embodiments, a total of 1 to 13 amino acids have been substituted, inserted and/or deleted in SEQ ID NO: 402. In certain embodiments, substitutions, insertions, or deletions occur in regions outside the CDRs (i.e., in the FRs). In a particular embodiment, the VH1 comprises one, two or three CDRs selected from the group consisting of: (a) a CDR-H1 comprising the amino acid sequence of SEQ ID NO:390, (b) a CDR-H2 comprising the amino acid sequence of SEQ ID NO:392, and (c) a CDR-H3 comprising the amino acid sequence of SEQ ID NO:395.

In some embodiments, the first antigen-binding fragment comprises a VL1 having at least 90%, 91%, 92%, 93%, 94%, 95%, 96%, 97%, 98%, 99%, or 100% sequence identity to the amino acid sequence of SEQ ID NO:403. In certain embodiments, a VL1 sequence contains substitutions (e.g., conservative substitutions), insertions, or deletions relative to the amino acid sequence of SEQ ID NO:403, but retains the same ability to bind CD3 as the antibody comprising SEQ ID NO:403. In certain embodiments, a total of 1 to 11 amino acids have been substituted, inserted and/or deleted in SEQ ID NO: 403. In certain embodiments, the substitutions, insertions, or deletions occur in regions outside the CDRs (i.e., in the FRs). In a particular embodiment, the VL1 comprises one, two or three CDRs selected from the group consisting of (a) a CDR-L1 comprising the amino acid sequence of SEQ ID NO:397; (b) a CDR-L2 comprising the amino acid sequence of SEQ ID NO:380; and (c) a CDR-L3 comprising the amino acid sequence of SEQ ID NO:400.

In some embodiments, the first antigen-binding fragment comprises a VH1 comprising a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395, and a VL1 comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400.

In some embodiments, the first antigen-binding fragment comprises a VH1 comprising the amino acid sequence of SEQ ID NO: 402, and a VL1 comprising the amino acid sequence of SEQ ID NO: 403.

In some embodiments, the first antigen-binding fragment comprises a VH1 comprising the CDR-H1, CDR-H2, and CDR-H3 of a VH having the sequence set forth in SEQ ID NO:402; and a VL1 comprising the CDR-L1, CDR-L2, and CDR-L3 of a VL having the sequence set forth in SEQ ID NO:403.

In some embodiments, the first antigen-binding fragment comprises one, two, three, four, five, or six CDRs of antibody TY25238 as shown in Table 7. In some embodiments, the first antigen-binding fragment comprises a VH1 and/or a VL1 of antibody TY25238 as shown in Table 8. In some embodiments, the first antigen-binding fragment comprises a scFv of antibody TY25238 as shown in Table 9. In some embodiments, the first antigen-binding fragment comprises a heavy chain of antibody TY25238 as shown in Table 12.

In some embodiments, the first antigen-binding fragment comprises a VH1 sequence having at least 90%, 91%, 92%, 93%, 94%, 95%, 96%, 97%, 98%, 99%, or 100% sequence identity to the amino acid sequence of SEQ ID NO: 410. In certain embodiments, a VH1 sequence contains substitutions (e.g., conservative substitutions), insertions, or deletions relative to the amino acid sequence of SEQ ID NO: 410, but retains the same ability to bind human CD3 as the antibody comprising SEQ ID NO: 410. In certain embodiments, a total of 1 to 13 amino acids have been substituted, inserted and/or deleted in SEQ ID NO: 410. In certain embodiments, substitutions, insertions, or deletions occur in regions outside the CDRs (i.e., in the FRs). In a particular embodiment, the VH1 comprises one, two or three CDRs selected from the group consisting of: (a) a CDR-H1 comprising the amino acid sequence of SEQ ID NO:390, (b) a CDR-H2 comprising the amino acid sequence of SEQ ID NO:394, and (c) a CDR-H3 comprising the amino acid sequence of SEQ ID NO:395.

In some embodiments, the first antigen-binding fragment comprises a VL1 having at least 90%, 91%, 92%, 93%, 94%, 95%, 96%, 97%, 98%, 99%, or 100% sequence identity to the amino acid sequence of SEQ ID NO:411. In certain embodiments, a VL1 sequence contains substitutions (e.g., conservative substitutions), insertions, or deletions relative to the amino acid sequence of SEQ ID NO: 411, but retains the same ability to bind human CD3 as the antibody comprising SEQ ID NO: 411. In certain embodiments, a total of 1 to 11 amino acids have been substituted, inserted and/or deleted in SEQ ID NO: 411. In certain embodiments, the substitutions, insertions, or deletions occur in regions outside the CDRs (i.e., in the FRs). In a particular embodiment, the VL1 comprises one, two or three CDRs selected from the group consisting of (a) a CDR-L1 comprising the amino acid sequence of SEQ ID NO:397; (b) a CDR-L2 comprising the amino acid sequence of SEQ ID NO:380; and (c) a CDR-L3 comprising the amino acid sequence of SEQ ID NO:381.

In some embodiments, the first antigen-binding fragment comprises a VH1 comprising a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395, and a VL1 comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381.

In some embodiments, the first antigen-binding fragment comprises a VH1 comprising the amino acid sequence of SEQ ID NO: 410, and a VL1 comprising the amino acid sequence of SEQ ID NO: 411.

In some embodiments, the first antigen-binding fragment comprises a VH1 comprising the CDR-H1, CDR-H2, and CDR-H3 of a VH having the sequence set forth in SEQ ID NO:410; and a VL1 comprising the CDR-L1, CDR-L2, and CDR-L3 of a VL having the sequence set forth in SEQ ID NO:411.

In some embodiments, the first antigen-binding fragment comprises the amino acid sequence of SEQ ID NO: 421 or SEQ ID NO: 422.

Any one of the masking moieties for anti-CD3 antibodies described herein may be used, including, for example, the masking moieties of section F. “Masking Moiety (MM)”, Tables B, 18-22, 13A and 40. In some embodiments, the first masking moiety (MM1) comprises an amino acid sequence according to Formula (IX): PYDDPDCPSHX₁SDCDX₂ (SEQ ID NO: 668), wherein X₁ is D or E, and X₂ is N or Q. In some embodiments, the first masking moiety (MM1) comprises an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y. In some embodiments, the first masking moiety (MM1) comprises the amino acid sequence of EVGSY (SEQ ID NO: 667) at the N-terminus of the MM1. In some embodiments, the first masking moiety (MM1) comprises the amino acid sequence of SEQ ID NO: 417 (EVGSYPYDDPDCPSHESDCDQ). In some embodiments, the first masking moiety (MM1) comprises the amino acid sequence of SEQ ID NO: 35. In some embodiments, the first masking moiety (MM1) comprises an amino acid sequence selected from the group consisting of SEQ ID NOs: 585-588. In some embodiments, the first masking moiety (MM1) comprises an amino acid sequence selected from the group consisting of SEQ ID NOs: 597-599.

In some embodiments, the masking efficiency of the MM1 is at least about any one of 2, 2.5, 5, 10, 20, 30, 40, 50, 60, 70, 80, 90, 100, 150, 200, 250, 300, 350, 400, 450, 490, 500, 510, 550, 600, 700, 800, 900, 1000, 1500, 2000, 2500, 3000, 4000, 5000, 10000, or more. In some embodiments, the masking efficiency of the MM1 is about any one of 2-10, 10-20, 20-50, 50-100, 40-510, 50-500, 100-200, 100-500, 200-500, 300-500, 400-500, 400-600, 500-1000, 1000-5000, 5000-10000, 10-100, 100-500, 100-1000, 1000-10000, 10-1000, or 100-10000. In some embodiments, the masking efficiency of the MM1 is at least 50. In some embodiments, the masking efficiency of the MM1 is at least about any one of 40, 41, 42, 43, 45, 46, 47, 48, 49, 50, 51, 52, 53, 54, 55, 56, 57, 58, 59, or 60. In some embodiments, the masking efficiency of the MM1 is 50-500. In some embodiments, the masking efficiency of the MM1 is 500. In some embodiments, masking efficiency is measured as the difference in affinity of an activatable antibody comprising the first masking moiety for binding its target (e.g., human CD3) before activation relative to the affinity of the affinity of a corresponding unmasked antibody (“parental antibody”) lacking the first masking moiety or the activatable antibody after activation for binding its target (e.g., human CD3). In some embodiments, masking efficiency is measured as the difference in activity (e.g., activation of NFAT promoter) of an activatable antibody comprising the first masking moiety for binding its target (e.g., human CD3) before activation relative to the activity of the parental antibody or the activatable antibody after activation. In some embodiments, masking efficiency is measured as the difference in the level of binding a cell expressing its target (e.g., a cell expressing human CD3) for an activatable antibody comprising the first masking moiety before activation relative to the activity of the parental antibody or the activatable antibody after activation. In some embodiments, the masking efficiency is measured by dividing the EC₅₀ of an activatable antibody comprising the first masking moiety before activation by the EC₅₀ of the parental antibody. The EC₅₀ values may be measured in an ELISA assay or a Jurkat NFAT reporter assay, see e.g., the methods of Example 3. In some embodiments, the masking efficiency is measured by dividing the k_{d} of an activatable antibody comprising the first masking moiety before activation by the k_{d} of the parental antibody.

Any one of the cleavage moieties described herein may be used, including, for example, the cleavable moieties of section G. “Cleavable Moiety (CM)” and Tables 13A, 18-22 and 40. In some embodiments, the first cleavable moiety (CM1) comprises an amino acid sequence selected from the group consisting of SEQ ID NOs: 77, 418, 420, 431 and 477-490, and 516-555. In some embodiments, the first cleavable moiety (CM1) comprises the amino acid sequence of SEQ ID NO: 418 (GGGPLGLAGGS). In some embodiments, the first cleavable moiety (CM1) comprises the amino acid sequence of SEQ ID NO: 77 (GGGPLGLAGSGGS).

The second antigen-binding fragment may specifically bind a target antigen, such as a tumor antigen. In some embodiments, the target antigen is a tumor antigen. In some embodiments, the target antigen is a tumor-associated antigen (TAA). In some embodiments, the target antigen is selected from the group consisting of CD19, CD20, EpCAM, CEA, PSMA, CD33, EGFR, HER2, EphA2, MCSP, ADAM17, PSCA, 17-A1, NKG2D, TROP2, CD79B, Nectin-4, BCMA, CD22, CD38, EGFR, GD2, SLAMF7, CD30, EpCAM, MUC1, MUC16, CD123, CD37, FOLR1, MET, FLT3, GPC3, CEACAM5, CLDN18, CSF1, Integrin alpha 5, NCAM1, PTPRC, CD138, NaPi2b, MSLN, DLL3, GPRC5D, GPNMB, ICAM1, SSTR2, carcinoma associated antigen CTAA16, CA9, ENG, ACVRL1, CD80, CSPG4, EGFL7, FLT1, HAVCR1, HGF, HLA-DRB, IGF1R, TPBG, ERBB3, and STEAP2. In some embodiments, the target antigen is HER2. In some embodiments, the target antigen is CD20. In some embodiments, the target antigen is TROP2. The second antigen-binding fragment may be derived from any one of the non-CD3 antibodies (e.g., anti-HER2 antibodies and anti-CD20 antibodies) described in section H. “Target binding moiety (TBM).”

In some embodiments, the second antigen-binding fragment is fused to a second masking moiety (MM2) via a second cleavable moiety (CM2). In some embodiments, the second antigen-binding fragment is not masked. In some embodiments, the second antigen-binding fragment is not fused to a second masking moiety. Any suitable masking moieties may be used, for example, anti-HER2 masking moieties described in Section F, “Masking Moiety (MM).” Any suitable cleavable moieties may be used, for example, cleavable moieties described in Section G, “Cleavable Moiety (CM).”

In some embodiments, the activatable multispecific antibody comprises a second antigen-binding fragment comprising a second immunoglobulin light chain variable domain (VL2) and a second immunoglobulin heavy chain variable domain (VH2) of an antibody that specifically binds HER2. In some embodiments, the second antigen-binding fragment comprises 1, 2, 3, 4, 5, or 6 CDRs of trastuzumab. In some embodiments, the second antigen-binding fragment comprises 1, 2, 3, 4, 5, or 6 CDRs as shown in Table 10. In some embodiments, the VH2 comprises a CDR-H1 comprising the amino acid sequence of SEQ ID NO: 423, a CDR-H2 comprising the amino acid sequence of SEQ ID NO: 424, and a CDR-H3 comprising the amino acid sequence of SEQ ID NO: 71, and the VL2 comprises a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the VH2 comprises the amino acid sequence of SEQ ID NO: 75, and the VL2 comprises the amino acid sequence of SEQ ID NO: 76. In some embodiments, the second antigen-binding fragment is fused to a second masking moiety (MM2) via a second cleavable moiety (CM2). In some embodiments, the MM2 comprises an amino acid sequence according to Formula (XI): ESX1X₂CX₃X₄DPFX₅CQX₆ (SEQ ID NO: 670), wherein X₁ is D or E, X₂ is A, F, V, or Y, X₃ is D or E, X₄ is A or L, X₅ is D or E, and X₆ is A, F, or Y. In some embodiments, the MM2 comprises an amino acid sequence according to Formula (XII): X₁X₂X₃X₄X₅X₆CX₇X₈DPYECX₉X₁₀ (SEQ ID NO: 671), wherein X₁ is A, H, or S, X₂ is A, D, or S, X₃ is A, T, or V, X₄ is P, S, or T, X₅ is D or E, X₆ is A or V, X₇ is D or E, X₈ is A or L, X₉ is Q, S, or T, and X₁₀ is A, H, or V. In some embodiments, the MM2 comprises an amino acid sequence according to Formula (XIII): YNSDDDCX₁SX₂YDPYTCYY (SEQ ID NO: 672), wherein X₁ is A, I, or V, and X₂ is H or R. In some embodiments, the MM2 comprises an amino acid sequence selected from the group consisting of SEQ ID NOs: 39, 419, 432-476, and 491-515. In some embodiments, the MM2 comprises the amino acid sequence of SEQ ID NO: 419 (ESDACDADPFDCQA). In some embodiments, the MM2 comprises the amino acid sequence of SEQ ID NO: 36. In some embodiments, the CM2 comprises an amino acid sequence selected from the group consisting of SEQ ID NOs: 77, 418, 420, 431 and 477-490, and 516-555. In some embodiments, the CM2 comprises the amino acid sequence of SEQ ID NO: 420. In some embodiments, the CM2 comprises the amino acid sequence of SEQ ID NO: 77.

In some embodiments, the activatable multispecific antibody comprises a second antigen-binding fragment comprising a second immunoglobulin light chain variable domain (VL2) and a second immunoglobulin heavy chain variable domain (VH2) of an antibody that specifically binds CD20. In some embodiments, the second antigen-binding fragment comprises 1, 2, 3, 4, 5, or 6 CDRs as shown in Table C. In some embodiments, the VH2 comprises a CDR-H1 comprising the amino acid sequence of SEQ ID NO: 556, a CDR-H2 comprising the amino acid sequence of SEQ ID NO: 557, and a CDR-H3 comprising the amino acid sequence of SEQ ID NO: 558, and the VL2 comprises a CDR-L1 comprising the amino acid sequence of SEQ ID NO: 559, a CDR-L2 comprising the amino acid sequence of SEQ ID NO: 560, and a CDR-L3 comprising the amino acid sequence of SEQ ID NO: 561. In some embodiments, the VH2 comprises the amino acid sequence of SEQ ID NO: 562, and the VL2 comprises the amino acid sequence of SEQ ID NO: 563.

In some embodiments, the activatable multispecific antibody comprises an Fc region. In some embodiments, the Fc region is of the human IgG1 subclass. In some embodiments, the Fc region is of the human IgG4 subclass. In some embodiments, the activatable multispecific antibody comprises any one of the Fc regions as described in Section J, “Fc regions and CH3 domains.” In some embodiments, the activatable multispecific antibody comprises any one of the CH3 domain mutations as described in Section J, “Fc regions and CH3 domains,” including mutations as described in Tables D-F.

In some embodiments, the activatable multispecific antibody comprises a first CH3 domain and a second CH3 domain, wherein the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions.

In some embodiments, the activatable multispecific antibody is a bispecific antibody. In some embodiments, the activatable multispecific antibody is an activatable BiTE molecule. Exemplary activatable BiTE molecules are shown, for example, in Tables 2 and 3A.

In some embodiments, the activatable multispecific antibody is an activatable BiTE targeting human CD3 and HER2.

In some embodiments, there is provided an activatable HER2xCD3 BiTE comprising a first polypeptide comprising the amino acid sequence of SEQ ID NO: 115, a second polypeptide comprising the amino acid sequence of SEQ ID NO: 116, and a third polypeptide comprising the amino acid sequence of SEQ ID NO: 117.

In some embodiments, there is provided an activatable HER2xCD3 BiTE comprising: a first polypeptide comprising the amino acid sequence of SEQ ID NO: 425, a second polypeptide comprising the amino acid sequence of SEQ ID NO: 426, and a third polypeptide comprising the amino acid sequence of SEQ ID NO: 112.

In some embodiments, there is provided an activatable HER2xCD3 BiTE comprising: a first polypeptide comprising the amino acid sequence of SEQ ID NO: 427, a second polypeptide comprising the amino acid sequence of SEQ ID NO: 428, and a third polypeptide comprising the amino acid sequence of SEQ ID NO: 112.

In some embodiments, there is provided an activatable HER2xCD3 BiTE comprising: a first polypeptide comprising the amino acid sequence of SEQ ID NO: 429, a second polypeptide comprising the amino acid sequence of SEQ ID NO: 430, and a third polypeptide comprising the amino acid sequence of SEQ ID NO: 115.

In some embodiments, there is provided an activatable HER2xCD3 BiTE comprising: a first polypeptide comprising the amino acid sequence of SEQ ID NO: 83, a second polypeptide comprising the amino acid sequence of SEQ ID NO: 84, and a third polypeptide comprising the amino acid sequence of SEQ ID NO: 85.

In some embodiments, there is provided an activatable HER2xCD3 BiTE comprising: a first polypeptide comprising the amino acid sequence of SEQ ID NO: 683, a second polypeptide comprising the amino acid sequence of SEQ ID NO: 684, and a third polypeptide comprising the amino acid sequence of SEQ ID NO: 685.

In some embodiments, the activatable multispecific antibody is an activatable BiTE targeting human CD3 and CD20.

In some embodiments, there is provided an activatable CD20xCD3 BiTE comprising: a first polypeptide comprising the amino acid sequence of SEQ ID NO: 564, a second polypeptide comprising the amino acid sequence of SEQ ID NO: 565, and a third polypeptide comprising the amino acid sequence of SEQ ID NO: 567.

In some embodiments, there is provided an activatable CD20xCD3 BiTE comprising: a first polypeptide comprising the amino acid sequence of SEQ ID NO: 564, a second polypeptide comprising the amino acid sequence of SEQ ID NO: 565, and a third polypeptide comprising the amino acid sequence of SEQ ID NO: 569.

In some embodiments, the activatable multispecific antibody is an activatable BiTE targeting human CD3 and TROP2.

In some embodiments, the activatable multispecific antibody is an activatable BiTE targeting human CD3 and BCMA.

In some embodiments, the activatable multispecific antibody is an activatable BiTE targeting human CD3 and CD19.

In some embodiments, the activatable multispecific antibody is cross-reactive with a CD3 polypeptide from at least one non-human species selected from the group consisting of cynomolgus monkey, mouse, rat and dog.

### B. Masked Multispecific Anti-CD3 Antibodies

As described above, the present disclosure is based in part on the discovery of anti-CD3 antibodies that bind CD3 with a relatively weak binding affinity (see, for example, FIGS. 21A-21C and Table 6), as well as masking moieties that block binding of the anti-CD3 antibodies with high efficiency (see, for example, Tables 4-5). In general, a masked antibody comprises a masking moiety that binds to the target-binding moiety of the antibody, thus reducing binding of the antibody to the target when the masking moiety is bound to the target-binding moiety. A masked antibody may contain a cleavable or a non-cleavable linker between the masking moiety and the antigen-binding fragment. Without wishing to be bound by theory, when the masked antibody contains a non-cleavable linker, it is believed that a masked antibody is in a state of dynamic equilibrium between a masked state in which the target-binding moiety is bound to the masking moiety, and a target-bound state in which the target-binding moiety is bound to the target. Accordingly, the relative binding affinities of the masking moiety for the target-binding moiety and the target-binding moiety for the target, as well as the local concentrations of the target and the masked antibody, determine the extent to which the antibody actually engages the target. Without wishing to be bound by theory, it is believed that masked multispecific antibodies with relatively weak affinities for CD3 and/or high masking efficiency for blocking CD3 binding have less severe side effects than traditional BiTE molecules. Due to this reduction in the severity of side effects, it is believed that the masked multispecific antibodies described herein allow for a greater therapeutic window. That is, masked multispecific antibodies described herein may be administered to treat disease effectively without producing toxic effects such as cytokine storm commonly associated with traditional BiTE molecules, e.g., BiTE molecules having stronger CD3 binding affinities.

In some embodiments, there is provided a multispecific antibody comprising: a) a first antigen-binding fragment comprising a VH1 and a VL1 of an anti-CD3 antibody that specifically binds CD3, wherein the first antigen-binding fragment is fused to a first masking moiety (MM1); and b) a second antigen-binding fragment comprising a VH2 and a VL2 of an antibody that specifically binds a target antigen (e.g., a tumor antigen, such as HER2, CD20, TROP2, BCMA, or CD19); wherein the MM1 is fused to the N-terminus of the VL1; wherein the MM1 competes with CD3 to specifically bind the CD3-binding moiety; wherein the multispecific antibody binds to CD3 via the first antigen-binding fragment; and wherein the first antigen-binding fragment binds CD3 with half-maximal binding at a concentration of antibody (EC₅₀) that is at least 10 nM (e.g., at least 100 nM) as determined by an ELISA assay (e.g., as described in Example 5). In some embodiments, the MM1 has a masking efficiency of at least 50 as determined by a Jurkat NFAT reporter assay (e.g., the assay in Example 3). In some embodiments, the first antigen-binding fragment is selected from the group consisting of a Fab, a Fv, a scFab and a scFv. In some embodiments, the first antigen-binding fragment is a scFv comprising, from N-terminus to C-terminus, VL1, an optional linker, and VH1.

In some embodiments, the masked multispecific antibody is an activatable antibody. In some embodiments, the multispecific antibody comprise a cleavable moiety. See, for example, activatable multispecific T cell engagers.

In some embodiments, the multispecific antibody is a not an activatable multispecific antibody. In some embodiments, the multispecific antibody does not comprise a cleavable moiety. In some embodiments, the first antigen-binding fragment comprises a first immunoglobulin light chain variable domain (VL1) and a first immunoglobulin heavy chain variable domain (VH1) of an anti-CD3 antibody, and wherein the MM1 is fused to the N-terminus of the VL1 via a first non-cleavable linker (NCL1). In some embodiments, the NCL1 is any one of the non-cleavable linkers known in the art. In some embodiments, the NCL1 is any one of the non-cleavable linkers described herein in Section I. Linker.

In some embodiments, there is provided a multispecific antibody comprising a first polypeptide, a second polypeptide, and a third polypeptide, wherein:
(i) the first polypeptide comprises a structure represented by the formula:

VH2-CH1-hinge-CH2-first CH3 (1a);

(ii) the second polypeptide comprises a structure represented by the formula:

MM1-NCL1-VL1-VH1-hinge-CH2-second CH3 (1b);

and
(iii) the third polypeptide comprises a structure represented by the formula:

VL2-CL (1c);

wherein:
   VL1 is a first immunoglobulin light chain variable domain;
   VH1 is a first immunoglobulin heavy chain variable domain;
   VL2 is a second immunoglobulin light chain variable domain;
   VH2 is a second immunoglobulin heavy chain variable domain;
   CL is an immunoglobulin light chain constant domain;
   CH1 is an immunoglobulin heavy chain constant domain 1;
   CH2 is an immunoglobulin heavy chain constant domain 2;
   first CH3 is a first immunoglobulin heavy chain constant domain 3;
   second CH3 is a second immunoglobulin heavy chain constant domain 3;
   hinge is an immunoglobulin hinge region connecting the CH1 and CH2 domains;
   MM1 is a masking moiety; and
   NCL1 is a non-cleavable linker;
      wherein VH1 and VL1 associate to form a scFv that specifically binds CD3 with half-maximal binding at a concentration of antibody (EC₅₀) that is at least 10 nM (e.g., at least 100 nM) as determined by an ELISA assay (e.g., as described in Example 5), wherein VH2 and VL2 associate to form a Fv that specifically binds a target antigen (e.g., a tumor antigen, such as HER2, CD20, TROP2, BCMA, or CD19), wherein the MM1 competes with CD3 to specifically bind the CD3-binding moiety; and wherein the multispecific antibody binds to CD3 via the first antigen-binding fragment. In some embodiments, the MM1 has a masking efficiency of at least 50 as determined by a Jurkat NFAT reporter assay (e.g., the assay in Example 3). In some embodiments, the multispecific antibody (e.g., the second polypeptide thereof) comprises an amino acid linker between VL1 and VH1.

In some embodiments, there is provided a multispecific antibody comprising a first polypeptide, a second polypeptide, a third polypeptide, and a fourth polypeptide, wherein:
(i) the first polypeptide comprises a structure represented by the formula:

VH1-CH1-hinge-CH2-first CH3 (3a);

(ii) the second polypeptide comprises a structure represented by the formula:

VH2-CH1-hinge-CH2-second CH3 (3b);

(iii) the third polypeptide comprises a structure represented by the formula:

MM1-NCL1-VL1-CL (3c);

and
(iv) the fourth polypeptide comprises a structure represented by the formula:

VL2-CL (3d);

wherein:
VL1 is a first immunoglobulin light chain variable domain;
VH1 is a first immunoglobulin heavy chain variable domain;
VL2 is a second immunoglobulin light chain variable domain;
VH2 is a second immunoglobulin heavy chain variable domain;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains; and
MM1 is a masking moiety;
NCL1 is a non-cleavable linker;
   wherein VH1 and VL1 associate to form a first Fv that specifically binds CD3 with half-maximal binding at a concentration of antibody (EC₅₀) that is at least 10 nM (e.g., at least 100 nM) as determined by an ELISA assay (e.g., as described in Example 5); wherein VL2 and VH2 associate to form a second Fv that specifically binds a target antigen (e.g., a tumor antigen, such as HER2, CD20, TROP2, BCMA, or CD19), wherein the MM1 competes with CD3 to specifically bind the CD3-binding moiety; and wherein the multispecific antibody binds to CD3 via the first antigen-binding fragment. In some embodiments, the MM1 has a masking efficiency of at least 50 as determined by a Jurkat NFAT reporter assay (e.g., the assay in Example 3).

In some embodiments, there is provided a multispecific antibody comprising: a) a first antigen-binding fragment that specifically binds CD3, wherein the first antigen-binding fragment is fused to a first masking moiety (MM1); and b) a second antigen-binding fragment that specifically binds a target antigen (e.g., a tumor antigen, such as HER2, CD20, TROP2, BCMA, or CD19), wherein the second antigen-binding fragment is fused to a second masking moiety (MM2) via a cleavable moiety (CM); wherein the MM1 competes with CD3 to specifically bind the CD3-binding moiety; wherein the CM comprises a cleavage site; wherein the MM2 inhibits binding of the multispecific antibody to the target antigen when the CM is not cleaved; wherein the multispecific antibody binds the target antigen via the second antigen-binding fragment when the CM is cleaved; and wherein the first antigen-binding fragment binds CD3 with half-maximal binding at a concentration of antibody (EC₅₀) that is at least 10 nM (e.g., at least 100 nM) as determined by an ELISA assay (e.g., as described in Example 5). In some embodiments, the MM1 has a masking efficiency of at least 50 as determined by a Jurkat NFAT reporter assay (e.g., the assay in Example 3).

In some embodiments, there is provided a multispecific antibody comprises a first polypeptide, a second polypeptide, and a third polypeptide, wherein:
(i) the first polypeptide comprises a structure represented by the formula:

VH2-CH1-hinge-CH2-first CH3 (2a);

(ii) the second polypeptide comprises a structure represented by the formula:

MM1-NCL1-VL1-VH1-hinge-CH2-second CH3 (2b);

and
(iii) the third polypeptide comprises a structure represented by the formula:

MM2-NCL2-VL2-CL (2c);

wherein:
   VL1 is a first immunoglobulin light chain variable domain;
   VH1 is a first immunoglobulin heavy chain variable domain;
   VL2 is a second immunoglobulin light chain variable domain;
   VH2 is a second immunoglobulin heavy chain variable domain;
   CL is an immunoglobulin light chain constant domain;
   CH1 is an immunoglobulin heavy chain constant domain 1;
   CH2 is an immunoglobulin heavy chain constant domain 2;
   first CH3 is a first immunoglobulin heavy chain constant domain 3;
   second CH3 is a second immunoglobulin heavy chain constant domain 3;
   hinge is an immunoglobulin hinge region connecting the CH1 and CH2 domains;
   MM1 is a first masking moiety;
   NCL1 is a first non-cleavable linker;
   MM2 is a second masking moiety; and
   NCL2 is a second non-cleavable linker;

wherein VH1 and VL1 associate to form a scFv that specifically binds CD3 with half-maximal binding at a concentration of antibody (EC₅₀) that is at least 10 nM (e.g., at least 100 nM) as determined by an ELISA assay (e.g., as described in Example 5), wherein VH2 and VL2 associate to form a Fv that specifically binds a target antigen (e.g., a tumor antigen, such as HER2, CD20, TROP2, BCMA, or CD19), wherein the MM1 competes with CD3 to specifically bind the CD3-binding moiety; wherein the multispecific antibody binds to CD3 via the first antigen-binding fragment; wherein the MM2 inhibits binding of the multispecific antibody to the target antigen; and wherein the multispecific antibody binds the target antigen via the second antigen-binding fragment. In some embodiments, there is provided a multispecific antibody comprises a first polypeptide, a second polypeptide, and a third polypeptide, wherein:
(i) the first polypeptide comprises a structure represented by the formula:

VH2-CH1-hinge-CH2-first CH3 (2a);

(ii) the second polypeptide comprises a structure represented by the formula:

MM1-NCL1-VL1-VH1-hinge-CH2-second CH3 (2b);

and
(iii) the third polypeptide comprises a structure represented by the formula:

MM2-CM-VL2-CL (2c);

wherein:
   VL1 is a first immunoglobulin light chain variable domain;
   VH1 is a first immunoglobulin heavy chain variable domain;
   VL2 is a second immunoglobulin light chain variable domain;
   VH2 is a second immunoglobulin heavy chain variable domain;
   CL is an immunoglobulin light chain constant domain;
   CH1 is an immunoglobulin heavy chain constant domain 1;
   CH2 is an immunoglobulin heavy chain constant domain 2;
   first CH3 is a first immunoglobulin heavy chain constant domain 3;
   second CH3 is a second immunoglobulin heavy chain constant domain 3;
   hinge is an immunoglobulin hinge region connecting the CH1 and CH2 domains;
   MM1 is a first masking moiety;
   NCL1 is a non-cleavable linker;
   MM2 is a second masking moiety;
   CM is a cleavable moiety comprising a cleavage site;
      wherein VH1 and VL1 associate to form a scFv that specifically binds CD3 with half-maximal binding at a concentration of antibody (EC₅₀) that is at least 10 nM (e.g., at least 100 nM) as determined by an ELISA assay (e.g., as described in Example 5), wherein VH2 and VL2 associate to form a Fv that specifically binds a target antigen (e.g., a tumor antigen, such as HER2, CD20, TROP2, BCMA, or CD19), wherein the MM1 competes with CD3 to specifically bind the CD3-binding moiety; wherein the multispecific antibody binds to CD3 via the first antigen-binding fragment; wherein the MM2 inhibits binding of the multispecific antibody to the target antigen when the CM is not cleaved; and wherein the multispecific antibody binds the target antigen via the second antigen-binding fragment when the CM is cleaved. In some embodiments, the MM1 has a masking efficiency of at least 50 as determined by a Jurkat NFAT reporter assay (e.g., the assay in Example 3). In some embodiments, the multispecific antibody (e.g., the second polypeptide thereof) comprises an amino acid linker between VL1 and VH1

In some embodiments, there is provided a multispecific antibody comprising a first polypeptide, a second polypeptide, a third polypeptide, and a fourth polypeptide, wherein:
(i) the first polypeptide comprises a structure represented by the formula:

VH1-CH1-hinge-CH2-first CH3 (4a);

(ii) the second polypeptide comprises a structure represented by the formula:

VH2-CH1-hinge-CH2-second CH3 (4b);

(iii) the third polypeptide comprises a structure represented by the formula:

MM1-NCL1-VL1-CL (4c);

and
(iv) the fourth polypeptide comprises a structure represented by the formula:

MM2-NCL2-VL2-CL (4d);

wherein:
VL1 is a first immunoglobulin light chain variable domain;
VH1 is a first immunoglobulin heavy chain variable domain;
VL2 is a second immunoglobulin light chain variable domain;
VH2 is a second immunoglobulin heavy chain variable domain;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a first masking moiety;
NCL1 is a first non-cleavable linker;
MM2 is a second masking moiety; and
NCL2 is a first non-cleavable linker;
   wherein VH1 and VL1 associate to form a first Fv that specifically binds CD3 with half-maximal binding at a concentration of antibody (EC₅₀) that is at least 10 nM (e.g., at least 100 nM) as determined by an ELISA assay (e.g., as described in Example 5); wherein VL2 and VH2 associate to form a second Fv that specifically binds a target antigen (e.g., a tumor antigen, such as HER2, CD20, TROP2, BCMA, or CD19), wherein the MM1 competes with CD3 to specifically bind the CD3-binding moiety; wherein the multispecific antibody binds to CD3 via the first antigen-binding fragment; wherein the MM2 inhibits binding of the multispecific antibody to the target antigen when the CM is not cleaved; and wherein the multispecific antibody binds the target antigen via the second antigen-binding fragment when the CM is cleaved. In some embodiments, there is provided a multispecific antibody comprising a first polypeptide, a second polypeptide, a third polypeptide, and a fourth polypeptide, wherein:
(i) the first polypeptide comprises a structure represented by the formula:

VH1-CH1-hinge-CH2-first CH3 (4a);

(ii) the second polypeptide comprises a structure represented by the formula:

VH2-CH1-hinge-CH2-second CH3 (4b);

(iii) the third polypeptide comprises a structure represented by the formula:

MM1-NCL1-VL1-CL (4c);

and
(iv) the fourth polypeptide comprises a structure represented by the formula:

MM2-CM-VL2-CL (4d);

wherein:
VL1 is a first immunoglobulin light chain variable domain;
VH1 is a first immunoglobulin heavy chain variable domain;
VL2 is a second immunoglobulin light chain variable domain;
VH2 is a second immunoglobulin heavy chain variable domain;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a first masking moiety;
NCL1 is a non-cleavable linker;
MM2 is a second masking moiety;
CM is a cleavable moiety comprising a cleavage site;
   wherein VH1 and VL1 associate to form a first Fv that specifically binds CD3 with half-maximal binding at a concentration of antibody (EC₅₀) that is at least 10 nM (e.g., at least 100 nM) as determined by an ELISA assay (e.g., as described in Example 5); wherein VL2 and VH2 associate to form a second Fv that specifically binds a target antigen (e.g., a tumor antigen, such as HER2, CD20, TROP2, BCMA, or CD19), wherein the MM1 competes with CD3 to specifically bind the CD3-binding moiety; wherein the multispecific antibody binds to CD3 via the first antigen-binding fragment; wherein the MM2 inhibits binding of the multispecific antibody to the target antigen when the CM is not cleaved; and wherein the multispecific antibody binds the target antigen via the second antigen-binding fragment when the CM is cleaved. In some embodiments, the MM1 has a masking efficiency of at least 50 as determined by a Jurkat NFAT reporter assay (e.g., the assay in Example 3).

In some embodiments, the first antigen-binding fragment binds CD3 (e.g., human CD3) with weak binding affinity. In some embodiments, the first antigen-binding fragment binds CD3 with a relatively weak binding affinity relative to the K_{D} of a reference antibody for CD3. In some embodiments, the first antigen-binding fragment binds CD3 with a higher dissociation constant than the reference antibody for CD3. In some embodiments, the first antigen-binding fragment binds CD3 with a lower association constant than the reference antibody for CD3. In some embodiments, the reference antibody is SP34. In some embodiments, the binding affinity of the first antigen-binding fragment to CD3 is measured when the first antigen-binding fragment is present as an isolated antigen-binding fragment or as part of a monospecific antibody. In some embodiments, the binding affinity of the first antigen-binding fragment to CD3 is measured when the first antigen-binding fragment is present in a multispecific antibody.

In some embodiments, the first antigen-binding fragment binds CD3 (e.g., human CD3) with half-maximal binding at a concentration of antibody (EC₅₀) that is at least about any one of 9, 10, 11, 12, 13, 14, 15, 20, 30, 40, 50, 60, 70, 75, 80, 85, 90, 95, 100, 101, 102, 103, 104, 105, 106, 107, 108, 109, 110, 111, 112, 113, 114, 115, 116, 117, 118, 119, 120, 125, 130, 135, 140, 145, 150, 160, 175, 200, 250 or more nM as determined by an enzyme-linked immunosorbent assay (ELISA), including any value or range in between these values. In some embodiments, the first antigen-binding domain binds human CD3 with an EC₅₀ of about any one of 10-50, 50-100, 100-150, 150-200, 10-100, 10-110, 9-111, 10-115, 75-150, 100-150, 10-150, 10-200, 50-125, 10-20, 20-50, 50-75, 75-125, 90-120, 100-120, 100-110, 110-120, 50-150, 50-200, or 10-250 nM, as determined by an enzyme-linked immunosorbent assay (ELISA). In some embodiments, the EC₅₀ is determined by an ELISA measuring binding of an unmasked multispecific antibody to CD3 (e.g., human CD3 or human CD3δε). In some embodiments, the first antigen binding fragment is a scFv, and the EC₅₀ is determined by an ELISA measuring binding of an unmasked multispecific antibody to CD3 (e.g., human CD3 or human CD3δε). In some embodiments, the EC₅₀ is determined by an ELISA measuring binding of a parental multispecific antibody that lacks an MM to CD3 (e.g., human CD3 or human CD3δε). In some embodiments, the first antigen binding fragment is a scFv, and the EC₅₀ is determined by an ELISA measuring binding of a parental multispecific antibody that lacks an MM to CD3 (e.g., human CD3 or human CD3δε). In some embodiments, the EC₅₀ is determined by an ELISA measuring binding of an antigen-binding fragment that binds CD3 (e.g., an isolated anti-CD3 scFv or scFv-Fc fusion protein) to CD3 (e.g., human CD3 or human CD3δε).

In some embodiments, the first antigen-binding fragment binds CD3 (e.g., human CD3) with an EC₅₀ that is at least about any one of 2, 3, 4, 5, 6, 7, 8, 9, 10, 15, 20, 25, 30, 35, 40, 45, 50, 55, 60, 70, 75, 80, 85, 90, 95, 100, 110, 120, 130, 140, 150, 200, 300, 400, 500 times or more higher than the EC₅₀ of a reference antibody (e.g., SP34), including any value or range in between these values. In some embodiments, the first antigen-binding fragment binds CD3 (e.g., human CD3) with an EC₅₀ that is about any one of 2-10, 10-20, 20-30, 30-40, 40-50, 50-60, 60-75, 75-100, 100-200, 200-500, 2-5, 5-10, 5-20, 5-30, 5-40, 5-50, 5-55, 5-60, 10-20, 10-30, 10-40, 10-50, 10-60, 20-40, 20-55, 30-60, 10-30, or 5-100 times the EC₅₀ of a reference antibody (e.g., SP34). In some embodiments, the EC₅₀ of the first antigen-binding fragment and the reference antibody are measured under the same experimental conditions. In some embodiments, the EC₅₀ of the first antigen-binding fragment and the reference antibody are measured in the same antibody format. In some embodiments, the EC₅₀ is determined by measuring binding of an unmasked multispecific antibody and an unmasked multispecific reference antibody to CD3 (e.g., human CD3 or human CD3δε). In some embodiments, the unmasked multispecific reference antibody comprises a CD3-binding moiety corresponding to SP34 (e.g., comprising the six CDRs of SP34). In some embodiments, the EC₅₀ is determined by measuring binding of a parental multispecific antibody that lacks an MM and a reference parental multispecific antibody that lacks an MM to CD3 (e.g., human CD3 or human CD3δε). In some embodiments, the reference parental multispecific antibody that an MM comprises a CD3-binding moiety corresponding to SP34 (e.g., comprising the six CDRs of SP34). In some embodiments, the Kd of the first antigen-binding fragment binds CD3 and the EC₅₀ of the reference antibody are determined by an ELISA, such as the ELISA as described in Example 3. In some embodiments, the Kd of the first antigen-binding fragment binds CD3 and the EC₅₀ of the reference antibody are determined by a cell-based assay, such as a Jurkat NFAT reporter assay as described in Example 3.

In some embodiments, the first antigen-binding fragment binds CD3 (e.g., human CD3) with a relatively weak dissociation constant (Kd) compared to a reference antibody (e.g., SP34), such as at least about any one of2, 3, 4, 5, 6, 7, 8, 9, 10, 15, 20, 25, 30, 35, 40, 45, 50, 55, 60, 70, 75, 80, 85, 90, 95, 100, 110, 120, 130, 140, 150, 200, 300, 400, 500 times or more weaker than the Kd of the reference antibody, including any value or range in between these values. In some embodiments, the first antigen-binding fragment binds CD3 (e.g., human CD3) with a dissociation constant (Kd) that is about any one of 2-10, 10-20, 20-30, 30-40, 40-50, 50-60, 60-75, 75-100, 100-200, 200-500, 2-5, 5-10, 5-20, 5-30, 5-40, 5-50, 5-55, 5-60, 10-20, 10-30, 10-40, 10-50, 10-60, 20-40, 20-55, 30-60, 10-30, or 5-100 times weaker than the Kd of a reference antibody (e.g., SP34). In some embodiments, the Kd of the first antigen-binding fragment and the reference antibody are measured under the same experimental conditions. In some embodiments, the Kd of the first antigen-binding fragment and the reference antibody are measured in the same antibody format. In some embodiments, the Kd is determined by measuring binding of an unmasked multispecific antibody and an unmasked multispecific reference antibody to CD3 (e.g., human CD3 or human CD3δε). In some embodiments, the unmasked multispecific reference antibody comprises a CD3-binding moiety corresponding to SP34 (e.g., comprising the six CDRs of SP34). In some embodiments, the Kd is determined by measuring binding of a parental multispecific antibody that lacks an MM and a reference parental multispecific antibody that lacks an MM to CD3 (e.g., human CD3 or human CD3δε). In some embodiments, the reference parental multispecific antibody that lacks an MM comprises a CD3-binding moiety corresponding to SP34 (e.g., comprising the six CDRs of SP34). In some embodiments, the Kd of the first antigen-binding fragment binds CD3 and the Kd of the reference antibody are determined by an ELISA.

In some embodiments, the first antigen-binding fragment binds CD3 (e.g., human CD3) with a dissociation constant (Kd) of at least about any one of 10, 20, 30, 40, 50, 60, 70, 80, 90, 100, 125, 150, 175, 200, 250, 300, 400, 500 or more nM, including any value or range in between these values. In some embodiments, the first antigen-binding fragment binds CD3 (e.g., human CD3) with a dissociation constant (Kd) of at least about any one of 1, 10, or 100 PM, including any value or range in between these values (when in the activated form). In some embodiments, the first antigen-binding fragment binds CD3 (e.g., human CD3) with a dissociation constant (Kd) of about any one of 10-50, 50-100, 100-200, 200-500, 500-1000, 10-100, 100-500, 100-1000, 50-200, 50-250, 50-500 or 10-1000 nM.

In some embodiments, the first antigen-binding fragment binds CD3 (e.g., human CD3) with a relatively fast off-rate (k_{off}) compared to a reference antibody (e.g., SP34), such as at least about any one of 2, 5, 10, 20, 50, 100, 200 times or more faster than the k_{off} of the reference antibody, including any value or range in between these values.

In some embodiments, the first antigen-binding fragment binds CD3 (e.g., human CD3) with a relatively slow on-rate (kₒₙ) compared to a reference antibody (e.g., SP34), such as at least about any one of 2, 5, 10, 20, 50, 100, 200 times or more slower than the kₒₙ of the reference antibody, including any value or range in between these values.

In some embodiments, the first antigen-binding fragment binds CD3 (e.g., human CD3) with a relatively small dissociation constant (kd) compared to a reference antibody (e.g., SP34), such as at least about any one of 2, 5, 10, 20, 50, 100, 200 times or more smaller than the kd of the reference antibody, including any value or range in between these values.

In some embodiments, the first antigen-binding fragment binds CD3 (e.g., human CD3) with a relatively large association constant (kₐ) compared to a reference antibody (e.g., SP34), such as at least about any one of 2, 5, 10, 20, 50, 100, 200 times or more larger than the kₐ of the reference antibody.

Methods of measuring the ability of an antibody (e.g., a masked multispecific antibody) to bind an antigen are known in the art, including, without limitation, via BIAcore analysis, surface plasmon resonance, ELISAs, flow cytometry, and cell-based assays (e.g., measuring binding to Jurkat cells) (See e.g., Example 5 and Table 6). The EC₅₀, dissociation constant (k_{d}), affinity constant (kₐ), off-rate (k_{off}), and/or on-rate (kₒₙ) of binding to CD3 (e.g., human CD3) may be measured in various contexts. In some embodiments, binding to CD3 (e.g., human CD3) is measured using an antigen-binding fragment that binds CD3 (e.g., a scFv or scFv-Fc fusion protein). In some embodiments, binding to CD3 (e.g., human CD3) is measured using an unmasked multispecific antibody. In some embodiments, binding to CD3 (e.g., human CD3) is measured using an activatable antibody (e.g., activatable multispecific antibody) wherein the cleavable moiety associated with the anti-CD3 antigen-binding fragment is cleaved. In some embodiments, binding to human CD3δε is measured. In some embodiments, binding to human CD3δε fused with an Fc fragment is measured. In some embodiments, binding to Jurkat cells is measured.

In some embodiments, an ELISA is performed using human CD3 (ε and δ chain heterodimer) fused with human Fc fragment as a binding substrate. An exemplary ELISA method is as follows:
1. 2 μg/mL of human CD3 (ε and δ chain heterodimer) fused with human Fc fragment is prepared and used to coat AN ELISA plate at 2-8° C. overnight.
2. After washing and blocking, 50 μL serial diluted IgG (e.g., a first antigen-binding fragment, an unmasked multispecific antibody, or an activatable antibody (e.g., activatable multispecific antibody) wherein the cleavable moiety associated with the anti-CD3 antigen-binding fragment is cleaved) is added and incubated at 37° C. for 1 hour.
3. Plates are washed three times and then incubated with 50 μL/well TMB substrate at room temperature for about 20 minutes.
4. The reaction is stopped.
5. Absorbance at 450 nm is measured.
6. The concentration of each antibody that produced half-maximal binding to CD3εδ is determined as the EC₅₀ in nM.

The first antigen-binding fragment and/or the second antigen-binding fragment may be of any suitable format, including, but are not limited to, a Fab, a Fv, a scFab and a scFv. The antigen-binding fragment may have a single polypeptide chain, or two or more polypeptide chains. The masking moiety (e.g., MM1 or MM2) may be fused to the N-terminus of any one of the polypeptide chain of an antigen-binding fragment that has multiple polypeptide chains. In some embodiments, the masking moiety (e.g., MM1 or MM2) is fused to the N-terminus of a VL (e.g., VL1 or VL2) of the antigen-binding fragment. In some embodiments, the masking moiety (e.g., MM1 or MM2) is fused to the N-terminus of a VH (e.g., VH1 or VH2) of the antigen-binding fragment.

The first antigen-binding fragment may be derived from any one of the anti-CD3 antibodies described herein, which have an EC₅₀ of at least 10 nM (e.g., at least 100 nM) as determined by an ELISA assay (e.g., as described in Example 5). Any one of the anti-CD3 antibodies and antigen-binding fragments described in Section i) “Anti-CD3 antibody” and Tables 5B-5H may be used.

In some embodiments, the first antigen-binding fragment comprises one, two, three, four, five, or six CDRs of an antibody as shown in Table 7. In some embodiments, the first antigen-binding fragment of the multispecific antibody comprises one, two, three, four, five, or six CDRs of the anti-CD3 antibody TY24051, TY25236, TY25023, TY25024, TY25237, TY25228, TY25227, TY25230, TY25229, TY25238, TY25239, TY25243, TY25231, TY25244, TY25241, or TY25240, as shown in Table 7. In some embodiments, the first antigen-binding fragment comprises a VH1 and/or a VL1 as shown in Table 8. In some embodiments, the first antigen-binding fragment comprises a VH1 and/or a VL1 of the anti-CD3 antibody TY24051, TY25236, TY25023, TY25024, TY25237, TY25228, TY25227, TY25230, TY25229, TY25238, TY25239, TY25243, TY25231, TY25244, TY25241, or TY25240, as shown in Table 8.

In some embodiments, the first antigen-binding fragment comprises one, two, three, four, five, or six CDRs of antibody TY25023 as shown in Table 7. In some embodiments, the first antigen-binding fragment comprises a VH and/or a VL of antibody TY25023 as shown in Table 8. In some embodiments, the first antigen-binding fragment comprises a scFv of antibody TY25023 as shown in Table 9. In some embodiments, the first antigen-binding fragment comprises a heavy chain of antibody TY25023 as shown in Table 12.

In some embodiments, the first antigen-binding fragment comprises a VH1 sequence having at least 90%, 91%, 92%, 93%, 94%, 95%, 96%, 97%, 98%, 99%, or 100% sequence identity to the amino acid sequence of SEQ ID NO: 402. In certain embodiments, a VH1 sequence contains substitutions (e.g., conservative substitutions), insertions, or deletions relative to the amino acid sequence of SEQ ID NO:402, but retains the same ability to bind CD3 as the antibody comprising SEQ ID NO: 402. In certain embodiments, a total of 1 to 13 amino acids have been substituted, inserted and/or deleted in SEQ ID NO: 402. In certain embodiments, substitutions, insertions, or deletions occur in regions outside the CDRs (i.e., in the FRs). In a particular embodiment, the VH1 comprises one, two or three CDRs selected from the group consisting of: (a) a CDR-H1 comprising the amino acid sequence of SEQ ID NO:390, (b) a CDR-H2 comprising the amino acid sequence of SEQ ID NO:392, and (c) a CDR-H3 comprising the amino acid sequence of SEQ ID NO:395.

In some embodiments, the first antigen-binding fragment comprises a VL1 having at least 90%, 91%, 92%, 93%, 94%, 95%, 96%, 97%, 98%, 99%, or 100% sequence identity to the amino acid sequence of SEQ ID NO:403. In certain embodiments, a VL1 sequence contains substitutions (e.g., conservative substitutions), insertions, or deletions relative to the amino acid sequence of SEQ ID NO:403, but retains the same ability to bind CD3 as the antibody comprising SEQ ID NO:403. In certain embodiments, a total of 1 to 11 amino acids have been substituted, inserted and/or deleted in SEQ ID NO: 403. In certain embodiments, the substitutions, insertions, or deletions occur in regions outside the CDRs (i.e., in the FRs). In a particular embodiment, the VL1 comprises one, two or three CDRs selected from the group consisting of (a) a CDR-L1 comprising the amino acid sequence of SEQ ID NO:397; (b) a CDR-L2 comprising the amino acid sequence of SEQ ID NO:380; and (c) a CDR-L3 comprising the amino acid sequence of SEQ ID NO:400.

In some embodiments, the first antigen-binding fragment comprises a VH1 comprising a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395, and a VL1 comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400.

In some embodiments, the first antigen-binding fragment comprises a VH1 comprising the amino acid sequence of SEQ ID NO: 402, and a VL1 comprising the amino acid sequence of SEQ ID NO: 403.

In some embodiments, the first antigen-binding fragment comprises a VH1 comprising the CDR-H1, CDR-H2, and CDR-H3 of a VH having the sequence set forth in SEQ ID NO:402; and a VL1 comprising the CDR-L1, CDR-L2, and CDR-L3 of a VL having the sequence set forth in SEQ ID NO:403.

In some embodiments, the first antigen-binding fragment comprises one, two, three, four, five, or six CDRs of antibody TY25238 as shown in Table 7. In some embodiments, the first antigen-binding fragment comprises a VH1 and/or a VL1 of antibody TY25238 as shown in Table 8. In some embodiments, the first antigen-binding fragment comprises a scFv of antibody TY25238 as shown in Table 9. In some embodiments, the first antigen-binding fragment comprises a heavy chain of antibody TY25238 as shown in Table 12.

In some embodiments, the first antigen-binding fragment comprises a VH1 sequence having at least 90%, 91%, 92%, 93%, 94%, 95%, 96%, 97%, 98%, 99%, or 100% sequence identity to the amino acid sequence of SEQ ID NO: 410. In certain embodiments, a VH1 sequence contains substitutions (e.g., conservative substitutions), insertions, or deletions relative to the amino acid sequence of SEQ ID NO: 410, but retains the same ability to bind human CD3 as the antibody comprising SEQ ID NO: 410. In certain embodiments, a total of 1 to 13 amino acids have been substituted, inserted and/or deleted in SEQ ID NO: 410. In certain embodiments, substitutions, insertions, or deletions occur in regions outside the CDRs (i.e., in the FRs). In a particular embodiment, the VH1 comprises one, two or three CDRs selected from the group consisting of: (a) a CDR-H1 comprising the amino acid sequence of SEQ ID NO:390, (b) a CDR-H2 comprising the amino acid sequence of SEQ ID NO:394, and (c) a CDR-H3 comprising the amino acid sequence of SEQ ID NO:395.

In some embodiments, the first antigen-binding fragment comprises a VL1 having at least 90%, 91%, 92%, 93%, 94%, 95%, 96%, 97%, 98%, 99%, or 100% sequence identity to the amino acid sequence of SEQ ID NO:411. In certain embodiments, a VL1 sequence contains substitutions (e.g., conservative substitutions), insertions, or deletions relative to the amino acid sequence of SEQ ID NO: 411, but retains the same ability to bind human CD3 as the antibody comprising SEQ ID NO: 411. In certain embodiments, a total of 1 to 11 amino acids have been substituted, inserted and/or deleted in SEQ ID NO: 411. In certain embodiments, the substitutions, insertions, or deletions occur in regions outside the CDRs (i.e., in the FRs). In a particular embodiment, the VL1 comprises one, two or three CDRs selected from the group consisting of (a) a CDR-L1 comprising the amino acid sequence of SEQ ID NO:397; (b) a CDR-L2 comprising the amino acid sequence of SEQ ID NO:380; and (c) a CDR-L3 comprising the amino acid sequence of SEQ ID NO:381.

In some embodiments, the first antigen-binding fragment comprises a VH1 comprising a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395, and a VL1 comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381.

In some embodiments, the first antigen-binding fragment comprises a VH1 comprising the amino acid sequence of SEQ ID NO: 410, and a VL1 comprising the amino acid sequence of SEQ ID NO: 411.

In some embodiments, the first antigen-binding fragment comprises a VH1 comprising the CDR-H1, CDR-H2, and CDR-H3 of a VH having the sequence set forth in SEQ ID NO:410; and a VL1 comprising the CDR-L1, CDR-L2, and CDR-L3 of a VL having the sequence set forth in SEQ ID NO:411.

In some embodiments, the first antigen-binding fragment comprises the amino acid sequence of SEQ ID NO: 421 or SEQ ID NO: 422.

Any one of the masking moieties for anti-CD3 antibodies described herein may be used, including, for example, the masking moieties of section F. “Masking Moiety (MM)”, Table B, 13A, and 40. In some embodiments, the first masking moiety (MM1) comprises an amino acid sequence according to Formula (IX): PYDDPDCPSHX₁SDCDX₂ (SEQ ID NO: 668), wherein X₁ is D or E, and X₂ is N or Q. In some embodiments, the first masking moiety (MM1) comprises an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y. In some embodiments, the first masking moiety (MM1) comprises the amino acid sequence of EVGSY (SEQ ID NO: 667) at the N-terminus of the MM1. In some embodiments, the first masking moiety (MM1) comprises the amino acid sequence of SEQ ID NO: 417 (EVGSYPYDDPDCPSHESDCDQ). In some embodiments, the first masking moiety (MM1) comprises the amino acid sequence of SEQ ID NO: 35. In some embodiments, the first masking moiety (MM1) comprises an amino acid sequence selected from the group consisting of SEQ ID NOs: 585-588. In some embodiments, the first masking moiety (MM1) comprises an amino acid sequence selected from the group consisting of SEQ ID NOs: 597-599.

In some embodiments, the masking efficiency of the MM1 is at least about any one of 2, 2.5, 5, 10, 20, 30, 40, 50, 60, 70, 80, 90, 100, 150, 200, 250, 300, 350, 400, 450, 490, 500, 510, 550, 600, 700, 800, 900, 1000, 1500, 2000, 2500, 3000, 4000, 5000, 10000, or more. In some embodiments, the masking efficiency of the MM1 is about any one of 2-10, 10-20, 20-50, 50-100, 40-510, 50-500, 100-200, 100-500, 200-500, 300-500, 400-500, 400-600, 500-1000, 1000-5000, 5000-10000, 10-100, 100-500, 100-1000, 1000-10000, 10-1000, or 100-10000. In some embodiments, the masking efficiency of the MM1 is at least 50. In some embodiments, the masking efficiency of the MM1 is at least about any one of 40, 41, 42, 43, 45, 46, 47, 48, 49, 50, 51, 52, 53, 54, 55, 56, 57, 58, 59, or 60. In some embodiments, the masking efficiency of the MM1 is 50-500. In some embodiments, the masking efficiency of the MM1 is 500. In some embodiments, masking efficiency is measured as the difference in affinity of a masked antibody comprising the first masking moiety for binding its target (e.g., human CD3) relative to the affinity of the affinity of a corresponding unmasked antibody (“parental antibody”) lacking the first masking moiety for binding its target (e.g., human CD3). In some embodiments, masking efficiency is measured as the difference in activity (e.g., activation of NFAT promoter) of a masked antibody comprising the first masking moiety for binding its target (e.g., human CD3) relative to the activity of the parental antibody. In some embodiments, masking efficiency is measured as the difference in the level of binding a cell expressing its target (e.g., a cell expressing human CD3) for a masked antibody comprising the first masking moiety relative to the activity of the parental antibody. In some embodiments, the masking efficiency is measured by dividing the EC₅₀ of a masked antibody comprising the first masking moiety by the EC₅₀ of the parental antibody. The EC₅₀ values may be measured in an ELISA assay or a Jurkat NFAT reporter assay, see e.g., the methods of Example 3. In some embodiments, the masking efficiency is measured by dividing the k_{d} of a masked antibody comprising the first masking moiety before activation by the k_{d} of the parental antibody.

The second antigen-binding fragment may specifically bind a target antigen, such as a tumor antigen. In some embodiments, the target antigen is a tumor antigen. In some embodiments, the target antigen is a tumor-associated antigen (TAA). In some embodiments, the target antigen is selected from the group consisting of CD19, CD20, EpCAM, CEA, PSMA, CD33, EGFR, HER2, EphA2, MCSP, ADAM17, PSCA, 17-A1, NKG2D, TROP2, CD79B, Nectin-4, BCMA, CD22, CD38, EGFR, GD2, SLAMF7, CD30, EpCAM, MUC1, MUC16, CD123, CD37, FOLR1, MET, FLT3, GPC3, CEACAM5, CLDN18, CSF1, Integrin alpha 5, NCAM1, PTPRC, CD138, NaPi2b, MSLN, DLL3, GPRC5D, GPNMB, ICAM1, SSTR2, carcinoma associated antigen CTAA16, CA9, ENG, ACVRL1, CD80, CSPG4, EGFL7, FLT1, HAVCR1, HGF, HLA-DRB, IGF1R, TPBG, ERBB3, and STEAP2. In some embodiments, the target antigen is HER2. In some embodiments, the target antigen is CD20. In some embodiments, the target antigen is TROP2. The second antigen-binding fragment may be derived from any one of the non-CD3 antibodies (e.g., anti-HER2 antibodies and anti-CD20 antibodies) described in section H. “Target binding moiety (TBM).”

In some embodiments, the second antigen-binding fragment is not masked. In some embodiments, the second antigen-binding fragment is not fused to a second masking moiety. Any suitable masking moieties may be used, for example, anti-HER2 masking moieties described in Section F, “Masking Moiety (MM).” In some embodiments, the second antigen-binding fragment is fused to a second masking moiety (MM2) via a second cleavable moiety (CM2). Any suitable cleavable moieties may be used, for example, cleavable moieties described in Section G, “Cleavable Moiety (CM).” In some embodiments, the second antigen-binding fragment is fused to a second masking moiety (MM2) via a second non-cleavable linker (NCL2). Any suitable non-cleavable linker may be used, for example, non-cleavable linkers described in Section I, “Linker.”

In some embodiments, the second antigen-binding fragment is fused to a second masking moiety (MM2) via a second cleavable moiety (CM2). In some embodiments, the second antigen-binding fragment is not masked. In some embodiments, the second antigen-binding fragment is not fused to a second masking moiety. Any suitable masking moieties may be used, for example, anti-HER2 masking moieties described in Section F, “Masking Moiety (MM).” Any suitable cleavable moieties may be used, for example, cleavable moieties described in Section G, “Cleavable Moiety (CM).”

In some embodiments, the multispecific antibody comprises a second antigen-binding fragment comprising a second immunoglobulin light chain variable domain (VL2) and a second immunoglobulin heavy chain variable domain (VH2) of an antibody that specifically binds HER2. In some embodiments, the second antigen-binding fragment comprises 1, 2, 3, 4, 5, or 6 CDRs of trastuzumab. In some embodiments, the second antigen-binding fragment comprises 1, 2, 3, 4, 5, or 6 CDRs as shown in Table 10. In some embodiments, the VH2 comprises a CDR-H1 comprising the amino acid sequence of SEQ ID NO: 423, a CDR-H2 comprising the amino acid sequence of SEQ ID NO: 424, and a CDR-H3 comprising the amino acid sequence of SEQ ID NO: 71, and the VL2 comprises a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the VH2 comprises the amino acid sequence of SEQ ID NO: 75, and the VL2 comprises the amino acid sequence of SEQ ID NO: 76. In some embodiments, the second antigen-binding fragment is fused to a second masking moiety (MM2) via a second cleavable moiety (CM2). In some embodiments, the MM2 comprises an amino acid sequence according to Formula (XI): ESX1X₂CX₃X₄DPFX₅CQX₆ (SEQ ID NO: 670), wherein X₁ is D or E, X₂ is A, F, V, or Y, X₃ is D or E, X₄ is A or L, X₅ is D or E, and X₆ is A, F, or Y. In some embodiments, the MM2 comprises an amino acid sequence according to Formula (XII): X₁X₂X₃X₄X₅X₆CX₇X₈DPYECX₉X₁₀ (SEQ ID NO: 671), wherein X₁ is A, H, or S, X₂ is A, D, or S, X₃ is A, T, or V, X₄ is P, S, or T, X₅ is D or E, X₆ is A or V, X₇ is D or E, X₈ is A or L, X₉ is Q, S, or T, and X₁₀ is A, H, or V. In some embodiments, the MM2 comprises an amino acid sequence according to Formula (XIII): YNSDDDCX₁SX₂YDPYTCYY (SEQ ID NO: 672), wherein X₁ is A, I, or V, and X₂ is H or R. In some embodiments, the MM2 comprises an amino acid sequence selected from the group consisting of SEQ ID NOs: 39, 419, 432-476, and 491-515. In some embodiments, the MM2 comprises the amino acid sequence of SEQ ID NO: 419 (ESDACDADPFDCQA). In some embodiments, the MM2 comprises the amino acid sequence of SEQ ID NO: 36. In some embodiments, the CM2 comprises an amino acid sequence selected from the group consisting of SEQ ID NOs: 77, 418, 420, 431 and 477-490, and 516-555. In some embodiments, the CM2 comprises the amino acid sequence of SEQ ID NO: 420. In some embodiments, the CM2 comprises the amino acid sequence of SEQ ID NO: 77.

In some embodiments, the multispecific antibody comprises a second antigen-binding fragment comprising a second immunoglobulin light chain variable domain (VL2) and a second immunoglobulin heavy chain variable domain (VH2) of an antibody that specifically binds CD20. In some embodiments, the second antigen-binding fragment comprises 1, 2, 3, 4, 5, or 6 CDRs as shown in Table C. In some embodiments, the VH2 comprises a CDR-H1 comprising the amino acid sequence of SEQ ID NO: 556, a CDR-H2 comprising the amino acid sequence of SEQ ID NO: 557, and a CDR-H3 comprising the amino acid sequence of SEQ ID NO: 558, and the VL2 comprises a CDR-L1 comprising the amino acid sequence of SEQ ID NO: 559, a CDR-L2 comprising the amino acid sequence of SEQ ID NO: 560, and a CDR-L3 comprising the amino acid sequence of SEQ ID NO: 561. In some embodiments, the VH2 comprises the amino acid sequence of SEQ ID NO: 562, and the VL2 comprises the amino acid sequence of SEQ ID NO: 563.

In some embodiments, the multispecific antibody comprises an Fc region. In some embodiments, the Fc region is of the human IgG1 subclass. In some embodiments, the Fc region is of the human IgG4 subclass. In some embodiments, the multispecific antibody comprises any one of the Fc regions as described in Section J, “Fc regions and CH3 domains.” In some embodiments, the multispecific antibody comprises any one of the CH3 domain mutations as described in Section J, “Fc regions and CH3 domains,” including mutations as described in Tables D-F.

In some embodiments, the multispecific antibody comprises a first CH3 domain and a second CH3 domain, wherein the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions.

In some embodiments, the multispecific antibody is a bispecific antibody. In some embodiments, the multispecific antibody binds human CD3 and HER2. In some embodiments, the multispecific antibody binds human CD3 and CD20. In some embodiments, the multispecific antibody binds human CD3 and TROP2. In some embodiments, the multispecific antibody binds human CD3 and BCMA. In some embodiments, the multispecific antibody binds human CD3 and CD19.

In some embodiments, the multispecific antibody is cross-reactive with a CD3 polypeptide from at least one non-human species selected from the group consisting of cynomolgus monkey, mouse, rat and dog.

### C. Activatable Anti-CD3 Antibodies

Also provided herein are activatable antibodies (“activatable anti-CD3 antibodies”) that target CD3 (e.g., human CD3). The activatable antibodies may be derived from any anti-CD3 antibodies known in the art, including, but not limited to, SP34, OKT3, as well as variants, mutants and derivatives thereof.

The present application provides activatable antibodies, activatable antibody fragments, and polypeptides that target CD3, comprising a masking moiety (MM) comprising an amino acid sequence selected from the group consisting of SEQ ID NOs: 35, 417, 585-588, and 597-599.

The present application also provides activatable antibodies, activatable antibody fragments, and polypeptides that target CD3, comprising a masking moiety (MM) comprising the amino acid sequence of EVGSY (SEQ ID NO: 667) at the N-terminus of the MM. Further, the present application provides activatable antibodies, activatable antibody fragments, and polypeptides that target CD3, comprising a masking moiety (MM) comprising an amino acid sequence according to Formula (IX): PYDDPDCPSHX₁SDCDX₂ (SEQ ID NO: 668), wherein X₁ is D or E, and X₂ is N or Q. The present application also provides activatable antibodies, activatable antibody fragments, and polypeptides that target CD3, comprising a masking moiety (MM) comprising an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y.

i) Activatable Anti-CD3 Antibodies Derived from SP34 and Low-Affinity Mutants

The present application provides activatable antibodies, activatable antibody fragments, and polypeptides that target CD3, comprising a masking moiety (MM). In some embodiments, the activatable antibody comprises a MM comprising the amino acid sequence of EVGSY (SEQ ID NO: 667) at the N-terminus of the MM. In some embodiments, the activatable antibody comprises a MM comprising an amino acid sequence according to Formula (IX): PYDDPDCPSHX₁SDCDX₂ (SEQ ID NO: 668), wherein X₁ is D or E, and X₂ is N or Q. In some embodiments, the MM comprise an amino acid sequence selected from the group consisting of SEQ ID NOs: 35, 417 and 597-599. In some embodiments, the MM comprises the amino acid sequence of SEQ ID NO: 35. In some embodiments, the MM comprises the amino acid sequence of SEQ ID NO: 35 or 417.

In some embodiments, there is provided an antibody light chain comprising a polypeptide comprising, from N-terminus to C-terminus, a MM, a cleavable moiety (CM), and a target binding moiety (TBM), wherein the MM comprises an amino acid sequence selected from the group consisting of SEQ ID NOs: 35, 417, and 597-599; wherein the CM comprises at least a first cleavage site; and wherein the TBM comprises a VL of an anti-CD3 antibody.

In some embodiments, there is provided an antibody heavy chain comprising a polypeptide comprising, from N-terminus to C-terminus, a MM, a CM, and a TBM, wherein the MM comprises an amino acid sequence selected from the group consisting of SEQ ID NOs: 35, 417, and 597-599; wherein the CM comprises at least a first cleavage site; and wherein the TBM comprises a VH of an anti-CD3 antibody.

In some embodiments, there is provided an activatable antibody targeting CD3 comprising a first polypeptide comprising, from N-terminus to C-terminus, a MM, a CM, and a TBM, wherein the MM comprises an amino acid sequence selected from the group consisting of SEQ ID NOs: 35, 417, and 597-599, wherein the MM inhibits binding of the activatable antibody to CD3 when the CM is not cleaved; wherein the CM comprises at least a first cleavage site; wherein the TBM comprises a VL, and the activatable antibody further comprises a second polypeptide comprising a VH; and wherein the activatable antibody binds CD3 via the VH and VL when the CM is cleaved.

In some embodiments, there is provided an activatable antibody targeting CD3 comprising a first polypeptide comprising, from N-terminus to C-terminus, a MM, a CM, and a TBM, wherein the MM comprises an amino acid sequence selected from the group consisting of SEQ ID NOs: 35, 417, and 597-599, wherein the MM inhibits binding of the activatable antibody to CD3 when the CM is not cleaved; wherein the CM comprises at least a first cleavage site; wherein the TBM comprises a VH, and the activatable antibody further comprises a second polypeptide comprising a VL; and wherein the activatable antibody binds CD3 via the VH and VL when the CM is cleaved.

In some embodiments, there is provided an activatable antibody targeting CD3 comprising a first polypeptide comprising, from N-terminus to C-terminus, a MM, a CM, and a scFv of an anti-CD3 antibody, wherein the MM comprises an amino acid sequence selected from the group consisting of SEQ ID NOs: 35, 417, and 597-599, wherein the MM inhibits binding of the activatable antibody to CD3 when the CM is not cleaved; wherein the CM comprises at least a first cleavage site; and wherein the activatable antibody binds CD3 via the scFv when the CM is cleaved.

In some embodiments, there is provided an activatable antibody targeting CD3 comprising, from N-terminus to C-terminus, a masking moiety (MM), a cleavable moiety (CM), and a CD3-binding moiety, wherein: a) the CD3-binding moiety comprises a VL and the activatable antibody further comprises a second polypeptide comprising a VH; b) the CD3-binding moiety comprises a VH and the activatable antibody further comprises a second polypeptide comprising a VL; c) the CD3-binding moiety comprises from the N-terminus to the C-terminus, a VL and a VH; or d) the CD3-binding moiety comprise from the N-terminus to the C-terminus, a VH and a VL; wherein the CM comprises a cleavage site; wherein the MM inhibits binding of the activatable antibody to CD3 when the CM is not cleaved; wherein the activatable antibody binds CD3 via the VH and the VL when the CM is cleaved; and wherein the activatable antibody binds CD3 with half-maximal binding at a concentration of antibody (EC₅₀) that is at least 10 nM (e.g., at least 50 nM, or at least 100 nM) as determined by an enzyme-linked immunosorbent assay (ELISA). In some embodiments, the first antigen-binding fragment binds CD3 with a dissociation constant (Kd) of at least 50 nM. In some embodiments, the MM comprises an amino acid sequence selected from the group consisting of SEQ ID NOs: 35, 417, and 597-599.

Any one of the anti-CD3 antibodies and antigen-binding fragments that competitively bind to the same epitope as SP34, including anti-CD3 antibodies described in Section i) “Anti-CD3 antibody” and Tables 5B-5H may be used.

In some embodiments, the TBM (i.e., CD3 binding moiety) comprises a VH comprising a CDR-H1 comprising the amino acid sequence of SEQ ID NO: 61, a CDR-H2 comprising the amino acid sequence of SEQ ID NO: 62, and a CDR-H3 comprising the amino acid sequence of SEQ ID NO: 63; and/or a VL comprising a CDR-L1 comprising the amino acid sequence of SEQ ID NO: 64, a CDR-L2 comprising the amino acid sequence of SEQ ID NO: 65, and a CDR-L3 comprising the amino acid sequence of SEQ ID NO: 66. In some embodiments, the CD3 binding moiety comprises a VH comprising the amino acid sequence of SEQ ID NO: 67, and/or a VL comprising the amino acid sequence of SEQ ID NO: 68. In some embodiments, the CD3 binding moiety is a scFv comprising the amino acid sequence of SEQ ID NO: 79. In some embodiments, the MM comprises an amino acid sequence selected from the group consisting of SEQ ID NOs: 35, 417 and 597-599.

In some embodiments, the TBM (i.e., CD3 binding moiety) comprises a VH comprising a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and/or a VL comprising a CDR-L1 comprising the amino acid sequence of SEQ ID NO: 397, a CDR-L2 comprising the amino acid sequence of SEQ ID NO: 398, and a CDR-L3 comprising the amino acid sequence of SEQ ID NO: 400. In some embodiments, the CD3 binding moiety comprises a VH comprising the amino acid sequence of SEQ ID NO: 402, and/or a VL comprising the amino acid sequence of SEQ ID NO: 403. In some embodiments, the CD3 binding moiety is a scFv comprising the amino acid sequence of SEQ ID NO: 421. In some embodiments, the MM comprises an amino acid sequence selected from the group consisting of SEQ ID NOs: 35, 417 and 597-599.

In some embodiments, the TBM (i.e., CD3 binding moiety) comprises a VH comprising a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and/or a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the CD3 binding moiety comprises a VH comprising the amino acid sequence of SEQ ID NO: 410, and/or a VL comprising the amino acid sequence of SEQ ID NO: 411. In some embodiments, the CD3 binding moiety is a scFv comprising the amino acid sequence of SEQ ID NO: 422. In some embodiments, the MM comprises an amino acid sequence selected from the group consisting of SEQ ID NOs: 35, 417 and 597-599.

Any one of the masking moieties for anti-CD3 antibodies described herein may be used, including, for example, the masking moieties of section F, “Masking Moiety (MM)” and Table B, Table 13A and Table 40. In some embodiments, the MM comprises the amino acid sequence of SEQ ID NO: 417. In some embodiments, the MM comprises the amino acid sequence of SEQ ID NO: 35. In some embodiments, the MM comprises the amino acid sequence of SEQ ID NO: 597. In some embodiments, the MM comprises the amino acid sequence of SEQ ID NO: 598. In some embodiments, the MM comprises the amino acid sequence of SEQ ID NO: 599.

Any one of the cleavage moieties described herein may be used, including, for example, the cleavable moieties of section G, “Cleavable Moiety (CM)” and Tables 13A, 18-22 and 40. In some embodiments, the CM comprises an amino acid sequence selected from the group consisting of SEQ ID NOs: 77, 418, 420, 431 and 477-490, and 516-555. In some embodiments, the CM comprises the amino acid sequence of SEQ ID NO: 418. In some embodiments, the CM comprises the amino acid sequence of SEQ ID NO: 77.

In some embodiments, the activatable antibody targeting CD3 is a multispecific antibody, such as a bispecific antibody. In some embodiments, the activatable antibody targeting CD3 is a bispecific T cell engager (BiTE) molecule, which also targets a tumor antigen, such as HER2 or CD3.

In some embodiments, the activatable antibody comprises a light chain comprising an amino acid sequence of TY23105, TY23110, TY23115, or TY23118, as shown in Table 3D. In some embodiments, the activatable antibody comprises a light chain comprising an amino acid sequence selected from the group consisting of SEQ ID NOs: 589, 591, 593, and 595. In some embodiments, the activatable antibody comprises a heavy chain comprising an amino acid sequence of TY23105, TY23110, TY23115, or TY23118, as shown in Table 3D. In some embodiments, the activatable antibody comprises a heavy chain comprising an amino acid sequence selected from the group consisting of SEQ ID NOs: 590, 592, 594, and 596.

ii) Activatable Anti-CD3 Antibodies Derived from OKT3

Also provided herein are activatable antibodies, activatable antibody fragments, and polypeptides that target CD3, comprising a masking moiety (MM) comprising an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y. In some embodiments, the activatable antibody comprises a MM comprising an amino acid sequence selected from the group consisting of SEQ ID NO: 585-588. In some embodiments, the MM comprises the amino acid of SEQ ID NO: 585. In some embodiments, the MM comprises the amino acid of SEQ ID NO: 586. In some embodiments, the MM comprises the amino acid of SEQ ID NO: 587. In some embodiments, the MM comprises the amino acid of SEQ ID NO: 588.

In some embodiments, there is provided an antibody light chain comprising a polypeptide comprising, from N-terminus to C-terminus, a MM, a cleavable moiety (CM), and a target binding moiety (TBM), wherein the MM comprises an amino acid sequence selected from the group consisting of SEQ ID NO: 585-588; wherein the CM comprises at least a first cleavage site; and wherein the TBM comprises a VL of an anti-CD3 antibody.

In some embodiments, there is provided an antibody heavy chain comprising a polypeptide comprising, from N-terminus to C-terminus, a MM, a CM, and a TBM, wherein the MM comprises an amino acid sequence selected from the group consisting of SEQ ID NO: 585-588; wherein the CM comprises at least a first cleavage site; and wherein the TBM comprises a VH of an anti-CD3 antibody.

In some embodiments, there is provided an activatable antibody targeting CD3 comprising a first polypeptide comprising, from N-terminus to C-terminus, a MM, a CM, and a TBM, wherein the MM comprises an amino acid sequence selected from the group consisting of SEQ ID NO: 585-588, wherein the MM inhibits binding of the activatable antibody to CD3 when the CM is not cleaved; wherein the CM comprises at least a first cleavage site; wherein the TBM comprises a VL, and the activatable antibody further comprises a second polypeptide comprising a VH; and wherein the activatable antibody binds CD3 via the VH and VL when the CM is cleaved.

Any one of the anti-CD3 antibodies and antigen-binding fragments that competitively bind to the same epitope as OKT3, including anti-CD3 antibodies described in Table 3B may be used.

In some embodiments, there is provided an activatable antibody targeting CD3 comprising a first polypeptide comprising, from N-terminus to C-terminus, a MM, a CM, and a TBM, wherein the MM comprises an amino acid sequence selected from the group consisting of SEQ ID NO: 585-588, wherein the MM inhibits binding of the activatable antibody to CD3 when the CM is not cleaved; wherein the CM comprises at least a first cleavage site; wherein the TBM comprises a VH, and the activatable antibody further comprises a second polypeptide comprising a VL; and wherein the activatable antibody binds CD3 via the VH and VL when the CM is cleaved.

In some embodiments, there is provided an activatable antibody targeting CD3 comprising a first polypeptide comprising, from N-terminus to C-terminus, a MM, a CM, and a scFv of an anti-CD3 antibody, wherein the MM comprises an amino acid sequence selected from the group consisting of SEQ ID NO: 585-588, wherein the MM inhibits binding of the activatable antibody to CD3 when the CM is not cleaved; wherein the CM comprises at least a first cleavage site; and wherein the activatable antibody binds CD3 via the scFv when the CM is cleaved.

In some embodiments, an activatable antibody is provided comprising, from N-terminus to C-terminus, a masking moiety (MM), a cleavable moiety (CM), and a CD3-binding moiety, wherein: a) the CD3-binding moiety comprises a VL and the activatable antibody further comprises a second polypeptide comprising a VH; b) the CD3-binding moiety comprises a VH and the activatable antibody further comprises a second polypeptide comprising a VL; c) the CD3-binding moiety comprises from the N-terminus to the C-terminus, a VL and a VH; or d) the CD3-binding moiety comprises from the N-terminus to the C-terminus, a VH and a VL; and wherein the CM comprises a cleavage site; wherein the MM inhibits binding of the activatable antibody to CD3 when the CM is not cleaved; wherein the activatable antibody binds CD3 via the VH and the VL when the CM is cleaved; wherein the MM comprises an amino acid sequence selected from the group consisting of SEQ ID NO: 585-588.

In some embodiments, the anti-CD3 antigen-binding fragment is selected from the group consisting of a Fab, a Fv, a scFab and a scFv.

In some embodiments, the TBM (i.e., CD3 binding moiety) comprises a VH comprising a CDR-H1 comprising the amino acid sequence of SEQ ID NO: 368, a CDR-H2 comprising the amino acid sequence of SEQ ID NO: 369, and a CDR-H3 comprising the amino acid sequence of SEQ ID NO: 370; and/or a VL comprising a CDR-L1 comprising the amino acid sequence of SEQ ID NO: 371, a CDR-L2 comprising the amino acid sequence of SEQ ID NO: 372, and a CDR-L3 comprising the amino acid sequence of SEQ ID NO: 373. In some embodiments, the CD3 binding moiety comprises a VH comprising the amino acid sequence of SEQ ID NO: 366, and/or a VL comprising the amino acid sequence of SEQ ID NO: 367. In some embodiments, the MM comprises an amino acid sequence selected from the group consisting of SEQ ID NOs: 585-588.

Any one of the cleavage moieties described herein may be used, including, for example, the cleavable moieties of section G, “Cleavable Moiety (CM)” and Tables 13A, 18-22 and 40. In some embodiments, the CM comprises an amino acid sequence selected from the group consisting of SEQ ID NOs: 77, 418, 420, 431 and 477-490, and 516-555. In some embodiments, the CM comprises the amino acid sequence of SEQ ID NO: 431.

In some embodiments, the activatable antibody comprises a light chain comprising an amino acid sequence of TY23100, TY23101, TY23102, or TY23104, as shown in Table 3C. In some embodiments, the activatable antibody comprises a light chain comprising an amino acid sequence selected from the group consisting of SEQ ID NOs: 577, 579, 581, and 583. In some embodiments, the activatable antibody comprises a heavy chain comprising an amino acid sequence of TY23100, TY23101, TY23102, or TY23104, as shown in Table 3C. In some embodiments, the activatable antibody comprises a heavy chain comprising an amino acid sequence selected from the group consisting of SEQ ID NOs: 578, 580, 582, and 584.

### D. Activatable Anti-HER2 Antibodies

The present application provides activatable antibodies, activatable antibody fragments, and polypeptides that target HER2, comprising a masking moiety (MM) comprising an amino acid sequence selected from the group consisting of SEQ ID NOs: 36, 419, 432-476, and 491-515. In some embodiments, the MM comprises the amino acid sequence of SEQ ID NO: 36 or 419.

In some embodiments, there is provided an antibody light chain comprising a polypeptide comprising, from N-terminus to C-terminus, a MM, a cleavable moiety (CM), and a target binding moiety (TBM), wherein the MM comprises an amino acid sequence selected from the group consisting of SEQ ID NOs: 36, 419, 432-476, and 491-515; wherein the CM comprises at least a first cleavage site; and wherein the TBM comprises a VL of an anti-HER2 antibody. In some embodiments, the MM comprises the amino acid sequence of SEQ ID NO: 36 or 419.

In some embodiments, there is provided an antibody heavy chain comprising a polypeptide comprising, from N-terminus to C-terminus, a MM, a CM, and a TBM, wherein the MM comprises an amino acid sequence selected from the group consisting of SEQ ID NOs: 36, 419, 432-476, and 491-515; wherein the CM comprises at least a first cleavage site; and wherein the TBM comprises a VH of an anti-HER2 antibody. In some embodiments, the MM comprises the amino acid sequence of SEQ ID NO: 36 or 419.

In some embodiments, there is provided an activatable antibody targeting HER2 comprising a first polypeptide comprising, from N-terminus to C-terminus, a MM, a CM, and a TBM, wherein the MM comprises an amino acid sequence selected from the group consisting of SEQ ID NOs: 36, 419, 432-476, and 491-515, wherein the MM inhibits binding of the activatable antibody to HER2 when the CM is not cleaved; wherein the CM comprises at least a first cleavage site; wherein the TBM comprises a VL, and the activatable antibody further comprises a second polypeptide comprising a VH; and wherein the activatable antibody binds HER2 via the VH and VL when the CM is cleaved. In some embodiments, the MM comprises the amino acid sequence of SEQ ID NO: 36 or 419.

In some embodiments, there is provided an activatable antibody targeting HER2 comprising a first polypeptide comprising, from N-terminus to C-terminus, a MM, a CM, and a TBM, wherein the MM comprises an amino acid sequence selected from the group consisting of SEQ ID NOs: 36, 419, 432-476, and 491-515, wherein the MM inhibits binding of the activatable antibody to HER2 when the CM is not cleaved; wherein the CM comprises at least a first cleavage site; wherein the TBM comprises a VH, and the activatable antibody further comprises a second polypeptide comprising a VL; and wherein the activatable antibody binds HER2 via the VH and VL when the CM is cleaved. In some embodiments, the MM comprises the amino acid sequence of SEQ ID NO: 36 or 419.

In some embodiments, there is provided an activatable antibody targeting HER2 comprising a first polypeptide comprising, from N-terminus to C-terminus, a MM, a CM, and a scFv and an anti-HER2 antibody, wherein the MM comprises an amino acid sequence selected from the group consisting of SEQ ID NOs: 36, 419, 432-476, and 491-515, wherein the MM inhibits binding of the activatable antibody to HER2 when the CM is not cleaved; wherein the CM comprises at least a first cleavage site; and wherein the activatable antibody binds HER2 via the scFv when the CM is cleaved. In some embodiments, the MM comprises the amino acid sequence of SEQ ID NO: 36 or 419.

In some embodiments, the TBM (i.e., HER2-binding moiety) comprises a VH comprising an CDR-H1 comprising the amino acid sequence of SEQ ID NO: 69, a CDR-H2 comprising the amino acid sequence of SEQ ID NO: 70, and a CDR-H3 comprising the amino acid sequence of SEQ ID NO: 71; and/or a VL comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74.

In some embodiments, the TBM (i.e., HER2-binding moiety) comprises a VH comprising an CDR-H1 comprising the amino acid sequence of SEQ ID NO: 69 or a variant thereof comprising up to about 3 (such as about any of 1, 2, or 3) amino acid substitutions, a CDR-H2 comprising the amino acid sequence of SEQ ID NO: 70 or a variant thereof comprising up to about 3 (such as about any of 1, 2, or 3) amino acid substitutions, and a CDR-H3 comprising the amino acid sequence of SEQ ID NO: 71 or a variant thereof comprising up to about 3 (such as about any of 1, 2, or 3) amino acid substitutions; and/or a VL comprising a CDR-L1 comprising the amino acid sequence of SEQ ID NO: 72 or a variant thereof comprising up to about 3 (such as about any of 1, 2, or 3) amino acid substitutions, a CDR-L2 comprising the amino acid sequence of SEQ ID NO: 73 or a variant thereof comprising up to about 3 (such as about any of 1, 2, or 3) amino acid substitutions, and a CDR-L3 comprising the amino acid sequence of SEQ ID NO: 74 or a variant thereof comprising up to about 3 (such as about any of 1, 2, or 3) amino acid substitutions.

In some embodiments, the TBM (i.e., HER2-binding moiety) comprises a VH comprising an CDR-H1 comprising the amino acid sequence of SEQ ID NO: 423, a CDR-H2 comprising the amino acid sequence of SEQ ID NO: 424, and a CDR-H3 comprising the amino acid sequence of SEQ ID NO: 71; and/or a VL comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74.

In some embodiments, the TBM (i.e., HER2-binding moiety) comprises a VH comprising an CDR-H1 comprising the amino acid sequence of SEQ ID NO: 423 or a variant thereof comprising up to about 3 (such as about any of 1, 2, or 3) amino acid substitutions, a CDR-H2 comprising the amino acid sequence of SEQ ID NO: 424 or a variant thereof comprising up to about 3 (such as about any of 1, 2, or 3) amino acid substitutions, and a CDR-H3 comprising the amino acid sequence of SEQ ID NO: 71 or a variant thereof comprising up to about 3 (such as about any of 1, 2, or 3) amino acid substitutions; and/or a VL comprising a CDR-L1 comprising the amino acid sequence of SEQ ID NO: 72 or a variant thereof comprising up to about 3 (such as about any of 1, 2, or 3) amino acid substitutions, a CDR-L2 comprising the amino acid sequence of SEQ ID NO: 73 or a variant thereof comprising up to about 3 (such as about any of 1, 2, or 3) amino acid substitutions, and a CDR-L3 comprising the amino acid sequence of SEQ ID NO: 74 or a variant thereof comprising up to about 3 (such as about any of 1, 2, or 3) amino acid substitutions.

In some embodiments, the TBM (i.e., HER2-binding moiety) comprises a VH comprising the amino acid sequence of SEQ ID NO: 75 and/or a VL comprising the amino acid sequence of SEQ ID NO: 76.

In some embodiments, the TBM (i.e., HER2-binding moiety) comprises a VH comprising an amino acid sequence comprising at least 80% (e.g., at least any one of 85%, 87%, 90%, 91%, 92%, 93%, 94%, 95%, 96%, 97%, 98%, 99%, or more) sequence identity to the amino acid sequence of SEQ ID NO: 75 and/or a VL comprising an amino acid sequence comprising at least 80% (e.g., at least any one of 85%, 87%, 90%, 91%, 92%, 93%, 94%, 95%, 96%, 97%, 98%, 99%, or more) sequence identity to the amino acid sequence of SEQ ID NO: 76.

In some embodiments, the activatable antibody targeting HER2 is a multispecific antibody, such as a bispecific antibody. In some embodiments, the activatable antibody targeting HER2 is a bispecific T cell engager (BiTE) molecule, which also targets CD3.

### E. General Properties of Activatable Antibodies

The activatable antibodies, including activatable multispecific antibodies, activatable anti-CD3 antibodies and activatable anti-HER2 antibodies described herein may have one or more of the general properties described in this Section E.

In some embodiments, the activatable antibody comprises a polypeptide comprising a target-binding moiety (TBM), a cleavable moiety (CM), and a masking moiety (MM). In some embodiments, the TBM comprises an amino acid sequence that binds a target such as CD3, HER2, CD20, TROP2, BCMA, or CD19. In some embodiments, the TBM comprises an antigen-binding fragment (ABD) of an antibody. In some embodiments, the TBM is an antigen-binding fragment. In some embodiments, the TBM comprises an antibody light chain variable region (VL) and an antibody heavy chain variable region (VH), wherein the VH and VL forms a binding domain that binds the target in the absence of the MM. In some embodiments, the VH and VL are covalently linked, e.g., in a scFv. In some embodiments, the VH and VL form an Fv fragment. In some embodiments, the VH is linked to an antibody heavy chain constant region, and the VL is linked to an antibody light chain constant region. In some embodiments, the activatable antibody comprises an Fc region comprising any one or combination of the engineered disulfide bonds or salt bridges described herein. In some embodiments, the activatable antibody comprises an Fc region that does not comprise any one or combination of the engineered disulfide bonds or salt bridges described herein.

In some embodiments, the activatable antibody comprises a polypeptide comprising the structure, from N-terminus to C-terminus, of: masking moiety (MM)-cleavable moiety (CM)-VL, and the activatable antibody further comprises a second polypeptide comprising a VH (e.g., a Fab fragment). In some embodiments, the activatable antibody comprises a polypeptide comprising the structure, from N-terminus to C-terminus, of: masking moiety (MM)-cleavable moiety (CM)-VL-VH (e.g., a scFv). In some embodiments, the activatable antibody comprises a polypeptide comprising the structure, from N-terminus to C-terminus, of: masking moiety (MM)-cleavable moiety (CM)-VH, and the activatable antibody further comprises a second polypeptide comprising a VL (e.g., a Fab fragment). In some embodiments, the activatable antibody comprises a polypeptide comprising the structure, from N-terminus to C-terminus, of: masking moiety (MM)-cleavable moiety (CM)-VH-VL (e.g., a scFv).

In some embodiments, the activatable antibody comprises a polypeptide comprising the structure, from N-terminus to C-terminus, of: masking moiety (MM)-L1-cleavable moiety (CM)-L2-VL, and the activatable antibody further comprises a second polypeptide comprising a VH (e.g., a Fab fragment). In some embodiments, the activatable antibody comprises a polypeptide comprising the structure, from N-terminus to C-terminus, of: masking moiety (MM)-L1-cleavable moiety (CM)-L2-VL-L3-VH (e.g., a scFv). In some embodiments, the activatable antibody comprises a polypeptide comprising the structure, from N-terminus to C-terminus, of: masking moiety (MM)-cleavable moiety (CM)-L1-VH, and the activatable antibody further comprises a second polypeptide comprising a VL (e.g., a Fab fragment). In some embodiments, the activatable antibody comprises a polypeptide comprising the structure, from N-terminus to C-terminus, of: masking moiety (MM)-L1-cleavable moiety (CM)-L2-VH-L3-VL (e.g., a scFv). In some embodiments, L1, L2, and/or L3 are linkers. In some embodiments, each of L1, L2, and L3 is a linker that can independently be either a bond or a peptide linker having an independently selected length of 1 or more, 2 or more, 3 or more, 4 or more, 5 or more, 6 or more, 7 or more, 8 or more, 9 or more, or 10 or more amino acids.

In some embodiments, there is provided an activatable antibody comprising a first polypeptide comprising a first CH3 domain, a second polypeptide comprising a second CH3 domain, and a third polypeptide, wherein:
(i) the first polypeptide comprises a structure represented by the formula:

VH-CH1-hinge-CH2-first CH3;

(ii) the second polypeptide comprises a structure represented by the formula:

MM1-CM1-scFv-hinge-CH2-second CH3;

and
(iii) the third polypeptide comprises a structure represented by the formula:

MM2-CM2-VL-CL;

wherein:
VL is an immunoglobulin light chain variable domain;
VH is an immunoglobulin heavy chain variable domain;
scFv is a single-chain variable fragment;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
   wherein VL and VH associate to form a first Fv that specifically binds a first target; wherein the scFv specifically binds a second target; wherein MM1 inhibits the binding of the scFv to the first target when CM1 is not cleaved; and wherein MM2 inhibits the binding of the first Fv to the second target when CM2 is not cleaved. In some embodiments, the first CH3 domain and the second CH3 domain do not comprise any one or combination of the engineered disulfide bonds or salt bridges described herein. In some embodiments, the first CH3 domain and the second CH3 domain comprise any one or combination of the engineered disulfide bonds or salt bridges described herein. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution. In some embodiments, the first target is a tumor antigen (e.g., HER2, CD20, TROP2, BCMA, or CD19) and the second target is CD3 (e.g., CD3e). In some embodiments, the first target is CD3 (e.g., CD3e) and the second target is a tumor antigen (e.g., HER2, CD20, TROP2, BCMA, or CD19).

In some embodiments, there is provided an activatable antibody comprising a first polypeptide comprising a first CH3 domain, a second polypeptide comprising a second CH3 domain, and a third polypeptide, wherein:
(i) the first polypeptide comprises a structure represented by the formula:

MM1-CM1-VH-CH1-hinge-CH2-first CH3;

(ii) the second polypeptide comprises a structure represented by the formula:

MM2-CM2-scFv-hinge-CH2-second CH3;

and
(iii) the third polypeptide comprises a structure represented by the formula:

VL-CL;

wherein:
VL is an immunoglobulin light chain variable domain;
VH is an immunoglobulin heavy chain variable domain;
scFv is a single-chain variable fragment;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
   wherein VL and VH associate to form a first Fv that specifically binds a first target; wherein the scFv specifically binds a second target; wherein MM1 inhibits the binding of the first Fv to the first target when CM1 is not cleaved; and wherein MM2 inhibits the binding of the scFv to the second target when CM2 is not cleaved. In some embodiments, the first CH3 domain and the second CH3 domain do not comprise any one or combination of the engineered disulfide bonds or salt bridges described herein. In some embodiments, the first CH3 domain and the second CH3 domain comprise any one or combination of the engineered disulfide bonds or salt bridges described herein. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution. In some embodiments, the first target is a tumor antigen (e.g., HER2, CD20, TROP2, BCMA, or CD19) and the second target is CD3 (e.g., CD3e). In some embodiments, the first target is CD3 (e.g., CD3e) and the second target is a tumor antigen (e.g., HER2, CD20, TROP2, BCMA, or CD19).

In some embodiments, there is provided an activatable antibody comprising a first polypeptide comprising a first CH3 domain, a second polypeptide comprising a second CH3 domain, a third polypeptide, and a fourth polypeptide, wherein:
(i) the first polypeptide comprises a structure represented by the formula:

MM1-CM1-VH1-CH1-hinge-CH2-first CH3;

(ii) the second polypeptide comprises a structure represented by the formula:

MM2-CM2-VH2-CH1-hinge-CH2-second CH3;

(iii) the third polypeptide comprises a structure represented by the formula:

VL1-CL;

and
(iv) the fourth polypeptide comprises a structure represented by the formula:

VL2-CL;

wherein:
VL1 is a first immunoglobulin light chain variable domain;
VH1 is a first immunoglobulin heavy chain variable domain;
VL2 is a second immunoglobulin light chain variable domain;
VH2 is a second immunoglobulin heavy chain variable domain;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
   wherein VL1 and VH1 associate to form a first Fv that specifically binds a first target; wherein VL2 and VH2 associate to form a second Fv that specifically binds a second target; wherein MM1 inhibits the binding of the first Fv to the first target when CM1 is not cleaved; and wherein MM2 inhibits the binding of the second Fv to the second target when CM2 is not cleaved. In some embodiments, the first CH3 domain and the second CH3 domain do not comprise any one or combination of the engineered disulfide bonds or salt bridges described herein. In some embodiments, the first CH3 domain and the second CH3 domain comprise any one or combination of the engineered disulfide bonds or salt bridges described herein. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution. In some embodiments, the first target is a tumor antigen (e.g., HER2, CD20, TROP2, BCMA, or CD19) and the second target is CD3 (e.g., CD3e). In some embodiments, the first target is CD3 (e.g., CD3e) and the second target is a tumor antigen (e.g., HER2, CD20, TROP2, BCMA, or CD19).

In some embodiments, there is provided an activatable antibody comprising a first polypeptide comprising a first CH3 domain, a second polypeptide comprising a second CH3 domain, a third polypeptide, and a fourth polypeptide, wherein:
(i) the first polypeptide comprises a structure represented by the formula:

MM1-CM1-VH1-CH1-hinge-CH2-first CH3;

(ii) the second polypeptide comprises a structure represented by the formula:

VH2-CH1-hinge-CH2-second CH3;

(iii) the third polypeptide comprises a structure represented by the formula:

VL1-CL;

and
(iv) the fourth polypeptide comprises a structure represented by the formula:

MM2-CM2-VL2-CL;

wherein:
VL1 is a first immunoglobulin light chain variable domain;
VH1 is a first immunoglobulin heavy chain variable domain;
VL2 is a second immunoglobulin light chain variable domain;
VH2 is a second immunoglobulin heavy chain variable domain;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
   wherein VL1 and VH1 associate to form a first Fv that specifically binds a first target; wherein VL2 and VH2 associate to form a second Fv that specifically binds a second target; wherein MM1 inhibits the binding of the first Fv to the first target when CM1 is not cleaved; and wherein MM2 inhibits the binding of the second Fv to the second target when CM2 is not cleaved. In some embodiments, the first CH3 domain and the second CH3 domain do not comprise any one or combination of the engineered disulfide bonds or salt bridges described herein. In some embodiments, the first CH3 domain and the second CH3 domain comprise any one or combination of the engineered disulfide bonds or salt bridges described herein. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution. In some embodiments, the first target is a tumor antigen (e.g., HER2, CD20, TROP2, BCMA, or CD19) and the second target is CD3 (e.g., CD3e). In some embodiments, the first target is CD3 (e.g., CD3e) and the second target is a tumor antigen (e.g., HER2, CD20, TROP2, BCMA, or CD19).

In some embodiments, there is provided an activatable antibody comprising a first polypeptide comprising a first CH3 domain, a second polypeptide comprising a second CH3 domain, a third polypeptide, and a fourth polypeptide, wherein:
(i) the first polypeptide comprises a structure represented by the formula:

VH1-CH1-hinge-CH2-first CH3;

(ii) the second polypeptide comprises a structure represented by the formula:

VH2-CH1-hinge-CH2-second CH3;

(iii) the third polypeptide comprises a structure represented by the formula:

MM1-CM1-VL1-CL;

and
(iv) the fourth polypeptide comprises a structure represented by the formula:

MM2-CM2-VL2-CL;

wherein:
VL1 is a first immunoglobulin light chain variable domain;
VH1 is a first immunoglobulin heavy chain variable domain;
VL2 is a second immunoglobulin light chain variable domain;
VH2 is a second immunoglobulin heavy chain variable domain;
CL is an immunoglobulin light chain constant domain;
CH1 is an immunoglobulin heavy chain constant domain 1;
CH2 is an immunoglobulin heavy chain constant domain 2;
hinge is an immunoglobulin hinge region connecting the CH1 and CH2 domains;
MM1 is a first masking peptide;
MM2 is a second masking peptide;
CM1 is a first cleavable peptide; and
CM2 is a second cleavable peptide;
   wherein VL1 and VH1 associate to form a first Fv that specifically binds a first target; wherein VL2 and VH2 associate to form a second Fv that specifically binds a second target; wherein MM1 inhibits the binding of the first Fv to the first target when CM1 is not cleaved; and wherein MM2 inhibits the binding of the second Fv to the second target when CM2 is not cleaved. In some embodiments, the first CH3 domain and the second CH3 domain do not comprise any one or combination of the engineered disulfide bonds or salt bridges described herein. In some embodiments, the first CH3 domain and the second CH3 domain comprise any one or combination of the engineered disulfide bonds or salt bridges described herein. In some embodiments, the first CH3 domain comprises N390C substitution and the second CH3 domain comprises S400C substitution, or the first CH3 domain comprises S400C substitution and the second CH3 domain comprises N390C substitution. In some embodiments, the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions. In some embodiments, the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions. In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the activatable antibody comprises an IgG1 Fc region, such as an IgG1 Fc having an N297A substitution. In some embodiments, the first target is a tumor antigen (e.g., HER2, CD20, TROP2, BCMA, or CD19) and the second target is CD3 (e.g., CD3e). In some embodiments, the first target is CD3 (e.g., CD3e) and the second target is a tumor antigen (e.g., HER2, CD20, TROP2, BCMA, or CD19).

In some embodiments, the activatable antibody is designed based on any one of the multispecific antibodies described herein, e.g., by fusing a masking moiety (MM) to a target-binding moiety (TBM) in the multispecific antibody via a cleavable moiety (CM), wherein the MM inhibits binding of the TBM to its target when the CM is not cleaved. Activatable antibodies have been described, for example, in WO2019/149282, the contents of which are incorporated herein by reference in its entirety. The activatable antibody may comprise any one of the TBMs described in Section H, “Target binding moiety (TBM).” The activatable antibodies described herein may comprise one or more linkers described in Section I, “Linker”, e.g., disposed between MM and CM, CM and TBM, or TBM and hinge region of an Fc.

The MM refers to an amino acid sequence that, when the CM of the activatable antibody is intact (e.g., uncleaved by a corresponding enzyme, and/or containing an unreduced cysteine-cysteine disulfide bond), the MM interferes with or inhibits binding of the TBM to its target. In some embodiments, the MM interferes with or inhibits binding of the TBM to its target so efficiently that binding of the TBM to its target is extremely low and/or below the limit of detection (e.g., binding cannot be detected in an ELISA or flow cytometry assay). The amino acid sequence of the CM may overlap with or be included within the MM. It should be noted that for sake of convenience “activatable antibody” are used herein to refer to an activatable antibody or activatable antibody in both their uncleaved (or “native”) state, as well as in their cleaved state. It will be apparent to the ordinarily skilled artisan that in some embodiments a cleaved activatable antibody may lack an MM due to cleavage of the CM, e.g., by a protease, resulting in release of at least the MM (e.g., where the MM is not joined to the activatable antibody by a covalent bond (e.g., a disulfide bond between cysteine residues)).

The CM generally includes an amino acid sequence that is cleavable, for example, serves as the substrate for an enzyme and/or a cysteine-cysteine pair capable of forming a reducible disulfide bond. As such, when the terms “cleavage,” “cleavable,” “cleaved” and the like are used in connection with a CM, the terms encompass enzymatic cleavage, e.g., by a protease, as well as disruption of a disulfide bond between a cysteine-cysteine pair via reduction of the disulfide bond that can result from exposure to a reducing agent.

In some embodiments, the activatable antibodies do not induce ADCC effects. Methods of measuring ADCC effects are known in the art. In some embodiments, the activatable antibodies (when in active form or inactive form) do not ADCC effects by more than about 10% (do not induce ADCC by more than about 10%, more than about 5%, more than about 1%, more than about 0.1%, more than about 0.01%) relative to a control. In some embodiments, the activatable antibodies comprise an Fc region having reduced or no ADCC effects and/or reduced or no crosslinking effects. In some embodiments, the activatable antibodies comprise an Fc region having enhanced ADCC and/or crosslinking effects.

In some embodiments, the activatable antibodies (e.g., activatable BiTE molecules) are capable of inhibiting tumor cell growth and/or proliferation. In some embodiments, the tumor cell growth and/or proliferation is inhibited by at least 5% (e.g., at least 5%, at least 10%, at least 20%, at least 30%, at least 40%, at least 50%, at least 60%, at least 70%, at least 80%, at least 90%, or at least 99%) when contacted with the activatable antibodies and T cells relative to corresponding tumor cells not contacted with the activatable antibodies (or relative to corresponding tumor cells contacted with an isotype control antibody and T cells). In some embodiments, the activatable antibodies are capable of reducing tumor volume in a subject when the subject is administered the activatable antibodies. In some embodiments, the activatable antibodies (e.g., activatable BiTE molecules) are capable of reducing tumor volume in a subject by at least 5% (e.g., at least 5%, at least 10%, at least 20%, at least 30%, at least 40%, at least 50%, at least 60%, at least 70%, at least 80%, at least 90%, or at least 99%) relative to the initial tumor volume in the subject (e.g., prior to administration of the activatable antibodies; as compared to a corresponding tumor in a subject administered an isotype control antibody). Methods of monitoring tumor cell growth/proliferation, tumor volume, and/or tumor inhibition are known in the art.

In some embodiments, the activatable antibodies have therapeutic effect on a cancer. In some embodiments, the activatable antibodies reduce one or more signs or symptoms of a cancer. In some embodiments, a subject suffering from a cancer goes into partial or complete remission when administered the activatable antibodies.

### F. Masking Moiety (MM)

The masked antibodies, multispecific antibodies (e.g., masked multispecific antibodies) and activatable antibodies (e.g., activatable multispecific antibodies, activatable anti-CD3 antibodies, and activatable anti-HER2 antibodies) described herein comprise one, two or more masking moieties. Sequences of exemplary masking moieties are shown in Table B below. Masking moieties can be isolated from phage display libraries, for example, as described in WO2019/149282, which is incorporated herein by reference in its entirety. Sequences of exemplary masking moieties are also shown in Tables B, 13A, 18-22 and 40.

In some embodiments, the masked, multispecific and/or activatable antibody comprises a MM comprising the amino acid sequence of EVGSY (SEQ ID NO: 667) at the N-terminus of the MM.

In some embodiments, the masked, multispecific and/or activatable antibody comprises a MM comprising an amino acid sequence according to Formula (IX): PYDDPDCPSHX₁SDCDX₂ (SEQ ID NO: 668), wherein X₁ is D or E, and X₂ is N or Q. In some embodiments, the masked, multispecific and/or activatable antibody comprises a MM comprising an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y.

In some embodiments, the masked, multispecific and/or activatable antibody comprises a MM comprising an amino acid sequence according to Formula (XI): ESX1X₂CX₃X₄DPFX₅CQX₆ (SEQ ID NO: 670), wherein X₁ is D or E, X₂ is A, F, V, or Y, X₃ is D or E, X₄ is A or L, X₅ is D or E, and X₆ is A, F, or Y. In some embodiments, the masked, multispecific and/or activatable antibody comprises a MM comprising an amino acid sequence according to Formula (XII): X₁X₂X₃X₄X₅X₆CX₇X₈DPYECX₉X₁₀ (SEQ ID NO: 671), wherein X₁ is A, H, or S, X₂ is A, D, or S, X₃ is A, T, or V, X₄ is P, S, or T, X₅ is D or E, X₆ is A or V, X₇ is D or E, X₈ is A or L, X₉ is Q, S, or T, and X₁₀ is A, H, or V. In some embodiments, the masked, multispecific and/or activatable antibody comprises a MM comprising an amino acid sequence according to Formula (XIII): YNSDDDCX₁SX₂YDPYTCYY (SEQ ID NO: 672), wherein X₁ is A, I, or V, and X₂ is H or R.

In some embodiments, the masked, multispecific and/or activatable antibody comprises a MM comprising the amino acid sequence of SEQ ID NO: 417. In some embodiments, the masked, multispecific and/or activatable antibody comprises a MM comprising the amino acid sequence of SEQ ID NO: 35.

In some embodiments, the masked, multispecific and/or activatable antibody comprises a masking moiety comprising an amino acid sequence selected from the group consisting of SEQ ID NOs: 36, 419, 432-476, and 491-515. In some embodiments, the masked, multispecific and/or activatable antibody comprises a MM comprising the amino acid sequence of SEQ ID NO: 36. In some embodiments, the multispecific and/or activatable antibody comprises a MM comprising the amino acid sequence of SEQ ID NO: 419.

In some embodiments, the masked, multispecific and/or activatable antibody comprises a first masking moiety comprising the amino acid sequence of SEQ ID NO: 35 or 417, and a second masking moiety comprising an amino acid sequence selected from the group consisting of SEQ ID NOs: 36 or 419, 432-476, and 491-515. In some embodiments, the masked, multispecific and/or activatable antibody comprises a first MM comprising the amino acid sequence of SEQ ID NO: 35, and a second MM comprising the amino acid sequence of SEQ ID NO: 36. In some embodiments, the masked, multispecific and/or activatable antibody comprises comprising the amino acid sequence of SEQ ID NO: 419.

TABLE B
Masking Moieties.

| | Masking | |
|---|---|---|
| SEQ ID NO | moiety | Sequence |
| 668 | CD3 MM | PYDDPDCPSHX₁SDCDX₂ |
| | | X₁: D E |
| | | X₂: N Q |
| 669 | CD3 MM | X₁X₂X₃DX₄X₅CX₆X₇DX₈X₉X₁₀CX₁₁X₁₂ |
| | | X₁: A D |
| | | X₂: A D P |
| | | X₃: D H P |
| | | X₄: F P |
| | | X₅: D P |
| | | X₆: D P |
| | | X₇: A P |
| | | X₈: D N P |
| | | X₉: A N P |
| | | X₁₀: D H S |
| | | X₁₁: H P Y |
| | | X₁₂: N P Y |
| 670 | HER2 MM | ESX₁X₂CX₃X₄DPFX₅CQX₆ |
| | | X₁: D E |
| | | X₂: A F V Y |
| | | X₃: D E |
| | | X₄: A L |
| | | X₅: D E |
| | | X₆: A F Y |
| 671 | HER2 MM | X₁X₂X₃X₄X₅X₆CX₇X₈DPYECX₉X₁₀ |
| | | X₁: A H S |
| | | X₂: A D S |
| | | X₃: A T V |
| | | X₄: P S T |
| | | X₅: D E |
| | | X₆: A V |
| | | X₇: D E |
| | | X₈: A L |
| | | X₉: Q S T |
| | | X₁₀: A H V |
| 672 | HER2 MM | YNSDDDCX₁SX₂YDPYTCYY |
| | | X₁: A I V |
| | | X₂: H R |
| 35 | CD3 MM | EVGSYPYDDPDCPSHDSDCDN |
| 419 | HER2 MM | ESDACDADPFDCQA |
| 417 | CD3 MM | EVGSYPYDDPDCPSHESDCDQ |
| 585 | CD3 MM | EVGSYYDNYNDCDNYDDDCYY |
| 586 | CD3 MM | EVGSYNADYHQCSDVPTDCLD |
| 587 | CD3 MM | EVGSYDSYDYNCYHDHHTCHD |
| 588 | CD3 MM | EVGSYAYHDDDCPDDDYDCAS |
| 597 | CD3 MM | EVGSYDDPDFPCDPDDADCPN |
| 598 | CD3 MM | EVGSYAPHDPDCPADPPSCYP |
| 599 | CD3 MM | EVGSYDADDPDCPADNNHCHY |

In some embodiments, the masking peptide (MM) interferes with, obstructs, reduces the ability of, prevents, inhibits, or competes with the corresponding target binding moiety for binding to its target (e.g., an “inactive activatable antibody). In some embodiments, the masking peptide (MM) interferes with, obstructs, reduces, prevents, inhibits, or competes with the target binding moiety for binding to its target only when the antibody has not been activated (e.g., activated by a change in pH (increased or decreased), activated by a temperature shift (increased or decreased), activated after being contacted with a second molecule (such as a small molecule or a protein ligand), etc.). In some embodiments, activation induces cleavage of the cleavable moiety. In some embodiments, activation induces conformation changes in the polypeptide(s) (e.g., displacement of the MM), leading to the MM no longer preventing the activatable antibody from binding to its target. In some embodiments, the MM interferes with, obstructs, reduces the ability of, prevents, inhibits, or competes with the target binding moiety for binding to its target only when the cleavable moiety (CM) has not been cleaved by one or more proteases that cleave within the cleavable moiety (CM). In some embodiments, the MM has a masking efficiency of at least 2.0 (e.g., at least 2.0, at least 3.0, at least 4.0, at least 5.0, at least 6.0, at least 7.0, at least 8.0, at least 9.0, at least 10, at least 25, at least 50, at least 75, at least 100, at least 150, at least 200, at least 300, at least 400, at least 500, at least 600, at least 700, at least 800, at least 900, at least 1000, at least 1500, at least 2000, at least 2500, at least 3000, at least 4000, at least 5000, etc.) prior to activation. In some embodiments, masking efficiency is measured as the difference in affinity of an activatable antibody comprising the MM for binding its target (before activation) relative to the affinity of a polypeptide lacking the MM for binding its target (e.g., the difference in affinity for a target antigen (such as CD3 or HER2) of an activatable antibody comprising a MM (before activation) relative to a parental antibody lacking the MM, or the difference in affinity for a target antigen (such as CD3 or HER2) of an activatable antibody comprising a MM (before activation) relative to the affinity for the target antigen of the activatable antibody after activation). In some embodiments, the masking efficiency is measured by dividing the EC50 for binding of an activatable antibody comprising a MM (before activation) by the EC50 of the parental antibody (e.g., by measuring EC50 by ELISA). In some embodiments, masking efficiency is measured as the difference in affinity of an activatable antibody comprising the MM for binding its target before activation relative to the affinity of the activatable antibody comprising the MM for binding its target after activation (e.g., the difference in affinity for a target antigen (such as CD3 or HER2) of an activatable antibody before activation relative to the activatable antibody after activation). In some embodiments, the MM binds the target binding moiety (TBM), and prevents the activatable antibody from binding to its target (e.g., an “inactive” activatable antibody). In some embodiments, the MM has a dissociation constant for binding to the target binding moiety (TBM) that is greater than the dissociation constant of the target binding moiety (TBM) for its target. Dissociation constants can be measured, e.g., by techniques such as ELISA, surface plasmon resonance or Bio-Layer Interferometry (BLI), or flow cytometry.

In some embodiments, the MM does not interfere with, obstruct, reduce the ability of, prevent, inhibit, or compete with the target binding moiety (TBM) for binding to its target after the polypeptide has been activated (e.g., activated by treatment with one or more proteases that cleave within the cleavable moiety (CM), activated by a change in pH (increased or decreased), activated by a temperature shift (increased or decreased), activated after being contacted with a second molecule (such as an enzyme), etc.). In some embodiments, the MM does not interfere with, obstruct, reduce the ability of, prevent, inhibit, or compete with the target binding moiety (TBM) for binding to its target after the cleavable moiety (CM) has been cleaved by one or more proteases that cleave within the cleavable moiety (CM). In some embodiments, the MM has a masking efficiency of at most about 1.75 (e.g., at most about 1.75, at most about 1.5, at most about 1.4, at most about 1.3, at most about 1.2, at most about 1.1, at most about 1.0, at most about 0.9, at most about 0.8, at most about 0.7, at most about 0.6, or at most about 0.5, etc.) after activation (e.g., the relative affinity of the activatable antibody after activation as compared to the affinity of a parental antibody).

In some embodiments, any of the MMs described herein may further comprise one or more additional amino acid sequences (e.g., one or more polypeptide tags). Examples of suitable additional amino acid sequence may include, without limitation, purification tags (such as his-tags, flag-tags, maltose binding protein and glutathione-S-transferase tags), detection tags (such as tags that may be detected photometrically (e.g., red or green fluorescent protein, etc.)), tags that have a detectable enzymatic activity (e.g., alkaline phosphatase, etc.), tags containing secretory sequences, leader sequences, and/or stabilizing sequences, protease cleavage sites (e.g., furin cleavage sites, TEV cleavage sites, Thrombin cleavage sites), and the like. In some embodiments, the one or more additional amino acid sequences are at the N-terminus of the MM.

### G. Cleavable Moiety (CM)

The activatable antibodies (e.g., activatable multispecific antibodies, activatable anti-CD3 antibodies, and activatable anti-HER2 antibodies) comprises one or more CMs, each of which is disposed between a MM and a TBM.

In some embodiments, the CM comprises at least a first cleavage site (CS1) (e.g., a first protease cleavage site). In some embodiments, the first cleavage site is a first protease cleavage site. Any suitable protease cleavage site recognized and/or cleaved by any protease (e.g., a protease that is known to be co-localized with a target of a polypeptide comprising the CM) known in the art may be used, including, for example, a protease cleavage site recognized and/or cleaved by urokinase-type plasminogen activator (uPA); matrix metalloproteinases (e.g., MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-23, MMP-24, MMP-26, and/or MMP-27); Tobacco Etch Virus (TEV) protease; plasmin; Thrombin; PSA; PSMA; ADAMS/ADAMTS (e.g., ADAM 8, ADAM 9, ADAMIO, ADAM12, ADAMIS, ADAM17/TACE, ADAMDECI, ADAMTSI, ADAMTS4, and/or ADAMTS5); caspases (e.g., Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and/or Caspase-14); aspartate proteases (e.g., RACE and/or Renin); aspartic cathepsins (e.g., Cathepsin D and/or Cathepsin E); cysteine cathepsins (e.g., Cathepsin B, Cathepsin C, Cathepsin K, Cathepsin L, Cathepsin S, Cathepsin V/L2, and/or Cathepsin X/Z/P); cysteine proteinases (e.g., Cruzipain, Legumain, and/or Otubain-2); KLKs (e.g., KLK4, KLK5, KLK6, KLK7, KLK8, KLK10, KLK11, KLK13, and/or KLK14); metallo proteainases (e.g., Meprin, Neprilysin, PSMA, and/or BMP-1); serine proteases (e.g., activated protein C, Cathepsin A, Cathepsin G, Chymase, and/or coagulation factor proteases (such as FVIIa, FIXa, FXa, FXla, FXIIa)); elastase; granzyme B; guanidinobenzoatase; HtrAl; human neutrophil elastase; lactoferrin; marapsin; NS3/4A; PACE4; tPA; tryptase; type II transmembrane serine proteases (TTSPs) (e.g., DESC1, DPP-4, FAP, Hepsin, Matriptase-2, MT-SP1/Matriptase, TMPRSS2, TMPRSS3 and/or TMPRSS4); etc. In some embodiments, the first protease cleavage site is a cleavage site for a protease selected from uPA, MMP-1, MMP-2, MMP-3, MMP-8, MMP-9, MMP-14, TEV protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. In some embodiments, the first protease cleavage site is a cleavage site for a protease selected from uPA, MMP-2, MMP-9, and/or TEV protease. In some embodiments, the protease cleavage comprises an amino acid sequence selected from SGRSA (SEQ ID NO: 127), PLGLAG (SEQ ID NO: 128), and ENLYFQG (SEQ ID NO: 129).

In some embodiments, the cleavable moiety (CM) further comprises at least a second cleavage site (e.g., at least a second, at least a third, at least a fourth, at least a fifth, etc.). In some embodiments, the cleavable moiety (CM) further comprises a second cleavage site (CS2). In some embodiments, the second cleavage site is a second protease cleavage site. The second protease cleavage site may be any suitable protease cleavage site recognized and/or cleaved by any of the proteases described above. In some embodiments, the first (CS1) and second (CS2) cleavage sites are protease cleavage sites recognized and/or cleaved by the same protease. In some embodiments, the first (CS1) and second (CS2) cleavage sites are protease cleavage sites recognized and/or cleaved by different proteases (e.g., the first protease cleavage site is recognized and/or cleaved by uPA, and the second protease cleavage site is recognized and/or cleaved by MMP-2; the first protease cleavage site is recognized and/or cleaved by uPA, and the second protease cleavage site is recognized and/or cleaved by MMP-9; the first protease cleavage site is recognized and/or cleaved by uPA, and the second protease cleavage site is recognized and/or cleaved by TEV protease; etc.). In some embodiments, the at least second cleavage site (CS2) is C-terminal to the first linker (L1). In some embodiments, the cleavable moiety (CM) comprises a structure, from N-terminus to C-terminus, of: (CS1)-L1-(CS2).

In some embodiments, the cleavable moiety (CM) further comprises at least a second linker (e.g., at least a second, at least a third, at least a fourth, at least a fifth, etc.). In some embodiments, the cleavable moiety (CM) further comprises a second linker (L2). The second linker (L2) may be any suitable linker described above. In some embodiments, the first (L1) and second (L2) linkers are the same. In some embodiments, the first (L1) and second (L2) linkers are different. In some embodiments, the at least second linker (L2) is C-terminal to the second cleavage site (CS2). In some embodiments, the cleavable moiety (CM) comprises a structure, from N-terminus to C-terminus, of: (CS1)-L1-(CS2)-L2.

Exemplary cleavable moieties are shown in Tables 13A, 18-22 and 40. In some embodiments, the cleavable moiety comprises an amino acid sequence selected from the group consisting of SEQ ID NOs: 77, 127-129, 418, 420, 431 and 477-490, and 516-555. In some embodiments, the cleavable moiety comprises the amino acid sequence of SEQ ID NO: 418. In some embodiments, the cleavable moiety comprises the amino acid sequence of SEQ ID NO: 77. In some embodiments, the cleavable moiety comprises the amino acid sequence of SEQ ID NO: 420.

### H. Target Binding Moiety (TBM)

The antibodies (e.g., masked antibodies, multispecific antibodies, activatable multispecific antibodies, activatable anti-CD3 antibodies, and activatable anti-HER2 antibodies) described herein comprises one or more target binding moieties (TBM), such as a CD3-binding moiety, a HER2-binding moiety, a CD20-binding moiety, and a TROP2-binding moiety. In some embodiments, the TBM comprises an antibody light chain variable region (VL) and/or an antibody heavy chain variable region (VH). In some embodiments, the TBM comprises a VL. In some embodiments, the TBM comprises a VH. In some embodiments, a TBM comprises a VL and/or a VH specificity for any target of interest, including, for example, CD3, CD19, CD20, EpCAM, CEA, PSMA, CD33, EGFR, HER2, EphA2, MCSP, ADAM17, PSCA, 17-A1, NKG2D, TROP2, CD79B, Nectin-4, BCMA, CD22, CD38, EGFR, GD2, SLAMF7, CD30, EpCAM, MUC1, MUC16, CD123, CD37, FOLR1, MET, FLT3, GPC3, CEACAM5, CLDN18, CSF1, Integrin alpha 5, NCAM1, PTPRC, CD138, NaPi2b, MSLN, DLL3, GPRC5D, GPNMB, ICAM1, SSTR2, carcinoma associated antigen CTAA16, CA9, ENG, ACVRL1, CD80, CSPG4, EGFL7, FLT1, HAVCR1, HGF, HLA-DRB, IGF1R, TPBG, ERBB3, and STEAP2. In some embodiments, the TBM is an antigen-binding fragment.

In some embodiments, the TBM is a scFv comprising from the N-terminus to the C-terminus: VL-L1-VH, wherein L1 is a peptide linker. In some embodiments, the TBM is a scFv comprising from the N-terminus to the C-terminus: VH-L1-VL, wherein L1 is a peptide linker. In some embodiment, L1 comprises the amino acid sequence of SEQ ID NO: 82. In some embodiments, the TBM is a scFv comprising an engineered disulfide bond between VH and VL, such as between C44 of VH and C100 of VL, wherein the numbering is based on Kabat numbering. In some embodiments, the scFv comprises a first cysteine residue at position 44 in the VH and a second cysteine residue at position 100 in the VL, wherein the first cysteine residue and the second cysteine residue form a disulfide bond, and wherein the numbering is based on Kabat numbering.

In some embodiments, the TBM comprises a full-length antibody light chain and/or a full-length antibody heavy chain. The antibody light chain may be a kappa or lambda light chain. The antibody heavy chain may be in any class, such as IgG, IgM, IgE, IgA, or IgD. In some embodiments, the antibody heavy chain is in the IgG class, such as IgG1, IgG2, IgG3, or IgG4 subclass. An antibody heavy chain described herein may be converted from one class or subclass to another class or subclass using methods known in the art.

In some embodiments, the TBM specifically binds a cell surface antigen. In some embodiments, the cell surface antigen is an antigen on immune effector cells, such as T cells (e.g., helper T cells, cytotoxic T cells, memory T cells, etc.), B cells, macrophages, and Natural Killer (NK) cells. In some embodiments, the cell surface antigen is a T cell surface antigen, such as CD3.

In some embodiments, the cell surface antigen is a tumor antigen. Tumor antigens are proteins that are produced by tumor cells that can elicit an immune response, particularly T-cell mediated immune responses. In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, pl5; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

The activatable antibodies described herein may comprise TBMs derived from any suitable antibodies targeting antigens of interest. Table C below shows antibody CDRs, VH, VL, scFv sequences of exemplary TBMs described herein.

TABLE C
Exemplary antibody sequences

| SEQ | | |
|---|---|---|
| ID | | |
| NO | Antibody sequence | Amino acid sequence (underlined are CDR sequences) |
| 61 | CD3 CDR-H1 | FTFNTYAMN |
| 62 | CD3 CDR-H2 | GRIRSKYNNYATYYADSVKG |
| 63 | CD3 CDR-H3 | HGNFGNSYVSWFAY |
| 64 | CD3 CDR-L1 | GSSTGAVTTSNYAN |
| 65 | CD3 CDR-L2 | GTNKRAP |
| 66 | CD3 CDR-L3 | WYSNLWV |
| 67 | CD3 VH | EVQLVESGGGLVQPGGSLRLSCAASGFTFNTYAMNWVRQA |
| | | PGKGLEWVGRIRSKYNNYATYYADSVKGRFTISRDDSKNTLY |
| | | LQMNSLRAEDTAVYYCVRHGNFGNSYVSWFAYWGQGTLVT |
| | | VSS |
| 68 | CD3 VL | QAVVTQEPSLTVSPGGTVTLTCGSSTGAVTTSNYANWVQQKP |
| | | GQAPRGLIGGTNKRAPGVPARFSGSLLGGKAALTLSGAQPED |
| | | EAEYYCALWYSNLWVFGGGTKLTVL |
| 69 | HER2 CDR-H1 | FNIKDTYIH |
| 70 | HER2 CDR-H2 | ARIYPTNGYTRYADSVKG |
| 71 | HER2 CDR-H3 | WGGDGFYAMDY |
| 72 | HER2 CDR-L1 | RASQDVNTAVA |
| 73 | HER2 CDR-L2 | SASFLYS |
| 74 | HER2 CDR-L3 | QQHYTTPPT |
| 75 | HER2 VH | EVQLVESGGGLVQPGGSLRLSCAASGFNIKDTYIHWVRQAP |
| | | GKGLEWVARIYPTNGYTRYADSVKGRFTISADTSKNTAYLQ |
| | | MNSLRAEDTAVYYCSRWGGDGFYAMDYWGQGTLVTVSS |
| 76 | HER2 VL | DIQMTQSPSSLSASVGDRVTITCRASQDVNTAVAWYQQKPG |
| | | KAPKLLIYSASFLYSGVPSRFSGSRSGTDFTLTISSLQPEDFAT |
| | | YYCQQHYTTPPTFGQGTKVEIKR |
| 79 | anti-CD3 scFv | QAVVTQEPSLTVSPGGTVTLTCGSSTGAVTTSNYANWVQQK |
| | | PGQAPRGLIGGTNKRAPGVPARFSGSLLGGKAALTLSGAQPE |
| | | DEAEYYCALWYSNLWVFGGGTKLTVLRGGGGSGGGGSGG |
| | | GGSGGGGSEVQLVESGGGLVQPGGSLRLSCAASGFTFNTYA |
| | | MNWVRQAPGKGLEWVGRIRSKYNNYATYYADSVKGRFTIS |
| | | RDDSKNTLYLQMNSLRAEDTAVYYCVRHGNFGNSYVSWFA |
| | | YWGQGTLVTVSS |
| 83 | anti-CTLA4 scFv | EVQLVESGGGLVQPGGSLRLSCAASGYSISSGYHWSWIRQAP |
| | | GKGLEWLARIDWDDDKYYSTSLKSRLTISRDNSKNTLYLQL |
| | | NSLRAEDTAVYYCARSYVYFDYWGQGTLVTVSSGGGGSGG |
| | | GGSGGGGSGGGGSDIQLTQSPSSLSASVGDRVTITCRASQSV |
| | | RGRFLAWYQQKPGKAPKLLIYDASNRATGIPSRFSGSGSGTD |
| | | FTLTISSLQPEDFATYYCQQSSSWPPTFGQGTKVEIKR |
| 556 | Anti-CD20 CDR-H1 | SYNMH |
| 557 | Anti-CD20 CDR-H2 | AIYPGSGDTSYNQKFKG |
| 558 | Anti-CD20 CDR-H3 | STYYGGDWYFNV |
| 559 | Anti-CD20 CDR-L1 | RASSSVSYIH |
| 560 | Anti-CD20 CDR-L2 | ATSNLAS |
| 561 | Anti-CD20 CDR-L3 | QQWTSNPPT |
| 562 | Anti-CD20 VH | EVQLVQSGAEVKKPGSSVKVSCKASGYTFTSYNMHWVRQA |
| | | PGKGLEWIGAIYPGSGDTSYNQKFKGRVTITADKSTSTAYME |
| | | LSSLRSEDTAVYYCARSTYYGGDWYFNVWGQGTLVTVSS |
| 563 | Anti-CD20 VL | QIVLSQSPAILSASPGEKVTMTCRASSSVSYIHWFQQKPGKSP |
| | | KPWIYATSNLASGVPVRFSGSGSGTSYSLTISRVEAEDAATYY |
| | | CQQWTSNPPTFGGGTKVEIK |

In some embodiments, the TBM is an anti-CD3 antibody or antigen-binding fragment thereof, including, e.g., a VH, VL, scFv, light chain, or heavy chain (such as IgG1, IgG2, IgG4). Any of the known anti-CD3 antibodies may be used in the present invention, including but not limited to, the Cris-7 monoclonal antibody (Reinherz, E. L. et al. (eds.), Leukocyte typing II, Springer Verlag, New York, (1986)), BC3 monoclonal antibody (Anasetti et al. (1990) J. Exp. Med. 172:1691), OKT3 (Ortho multicenter Transplant Study Group (1985) N. Engl. J. Med. 313:337) and derivatives thereof such as OKT3 ala-ala (Herold et al. (2003) J. Clin. Invest. 11:409), visilizumab (Carpenter et al. (2002) Blood 99:2712), and 145-2C11 monoclonal antibody (Hirsch et al. (1988) J. Immunol. 140: 3766), Otelixizumab and Foralumab. Further CD3 binding molecules contemplated herein include UCHT-1 (Beverley, P C and Callard, R. E. (1981) Eur. J. Immunol. 11: 329-334, SP34 (Silvana et. al. (1985) The EMBO Journal. 4:337-344) and CD3 binding molecules described in WO2004/106380; WO2010/037838; WO2008/119567; WO2007/042261; WO2010/0150918; WO2018/052503; WO2016/204966. Additional anti-CD3 antibodies are described in section i) “Anti-CD3 antibody” below.

In some embodiments, the TBM comprises a VH comprising an CDR-H1 comprising the amino acid sequence of SEQ ID NO: 61, a CDR-H2 comprising the amino acid sequence of SEQ ID NO: 62, and/or a CDR-H3 comprising the amino acid sequence of SEQ ID NO: 63. In some embodiments, the TBM comprises a VL comprising a CDR-L1 comprising the amino acid sequence of SEQ ID NO: 64, a CDR-L2 comprising the amino acid sequence of SEQ ID NO: 65, and/or a CDR-L3 comprising the amino acid sequence of SEQ ID NO: 66. In some embodiments, the TBM comprises a VH comprising the amino acid sequence of SEQ ID NO: 67. In some embodiments, the TBM comprises a VL comprising the amino acid sequence of SEQ ID NO: 68. In some embodiments, the TBM comprises a scFv comprising the amino acid sequence of SEQ ID NO: 79.

In some embodiments, the TBM is an anti-HER2 antibody or antigen-binding fragment thereof, including, e.g., a VH, VL, scFv, light chain, or heavy chain (such as IgG1, IgG2, IgG4). Any of the known anti-HER2 antibodies may be used in the present invention, including but not limited to, trastuzumab (HERCEPTIN®) (1998, Cancer Res 58 (13):2825-2831), MDXH210 (Schwaab et al., 2001, Journal of Immunotherapy, 24(1):79-87), disitamab (Toxicol Lett. 2019. S0378-4274(19)₃₀₄₂₁-7), and pertuzumab (Agus D B, Gordon M S, Taylor C, et al. J Clin Oncol. 2005; 23(11):2534-2543). In some embodiments, the TBM is derived from trastuzumab or a biosimilar thereof.

In some embodiments, the TBM comprises a VH comprising an CDR-H1 comprising the amino acid sequence of SEQ ID NO: 69, a CDR-H2 comprising the amino acid sequence of SEQ ID NO: 70, and/or a CDR-H3 comprising the amino acid sequence of SEQ ID NO: 71. In some embodiments, the TBM comprises a VL comprising a CDR-L1 comprising the amino acid sequence of SEQ ID NO: 72, a CDR-L2 comprising the amino acid sequence of SEQ ID NO: 73, and/or a CDR-L3 comprising the amino acid sequence of SEQ ID NO: 74. In some embodiments, the TBM comprises a VH comprising the amino acid sequence of SEQ ID NO: 75. In some embodiments, the TBM comprises a VL comprising the amino acid sequence of SEQ ID NO: 76.

In some embodiments, the TBM is an anti-CD20 antibody or antigen-binding fragment thereof, including, e.g., a VH, VL, scFv, light chain, or heavy chain (such as IgG1, IgG2, IgG4). Any of the known anti-CD20 antibodies may be used in the present invention, including but not limited to, rituximab, ocrelizumab, obinutuzumab, ofatumumab, tositumomab, ublituximab, B-Lyl, 11B8, AT80, HI47, 2C6, 2F2, 2H7 and GA101, biosimilars thereof, and derivatives thereof. In some embodiments, the anti-CD20 antibody is a type I anti-CD20 antibody. In some embodiments, the anti-CD20 antibody is a type II anti-CD20 antibody. Anti-CD20 antibodies have been described, for example, in U.S. Pat. No. 7,879,984, WO2005/044859, WO2004/035607, WO2005/103081, WO2004/056312, WO2007/031875, and WO2015/095410. The teachings of each of the aforementioned publications are hereby incorporated by reference. In some embodiments, the TBM is derived from rituximab or a biosimilar thereof.

In some embodiments, the TBM comprises a VH comprising an CDR-H1 comprising the amino acid sequence of SEQ ID NO: 556, a CDR-H2 comprising the amino acid sequence of SEQ ID NO: 557, and a CDR-H3 comprising the amino acid sequence of SEQ ID NO: 558; and/or a VL comprising a CDR-L1 comprising the amino acid sequence of SEQ ID NO: 559, a CDR-L2 comprising the amino acid sequence of SEQ ID NO: 560, and a CDR-L3 comprising the amino acid sequence of SEQ ID NO: 561. In some embodiments, the TBM comprises a VH comprising the amino acid sequence of SEQ ID NO: 562 and/or a VL comprising the amino acid sequence of SEQ ID NO: 563.

In some embodiments, the TBM is an anti-TROP2 antibody or antigen-binding fragment thereof, including, e.g., a VH, VL, scFv, light chain, or heavy chain (such as IgG1, IgG2, IgG4). Any of the known anti-TROP2 antibodies may be used in the present invention, including, but not limited to, Datopotamab (Daiichi Sankyo Inc.), GA733, hRS7, BR110, IDAC Accession No. 141205-03, biosimilars thereof, and derivatives thereof. Anti-TROP2 antibodies have been described, for example, in U.S. Pat. Nos. 6,653,104, 5,840,854, 7,420,040, 7,420,041, and 9,670,287.

In some embodiments, the TBM is an anti-BCMA antibody or antigen-binding fragment thereof, including, e.g., a VH, VL, scFv, light chain, or heavy chain (such as IgG1, IgG2, IgG4). Any of the known anti-BCMA antibodies may be used in the present invention, including, but not limited to, belantamab mafodotin (GSK2857916), MEDI2228, CC-99712, 4C8A, the anti-BCMA regions of teclistamab, pavurutamab, pacanalotama, and balnuctamab, biosimilars thereof, and derivatives thereof. Anti-BCMA antibodies have been described, for example, in International Publication Nos. WO2001024811A1, WO2002066516A2, WO2010104949A2, U.S. Pat. No. 7,083,785 and Gras, M. P. et al. Int Immunol. 1995 July; 7(7):1093-106).

In some embodiments, the TBM is an anti-CD19 antibody or antigen-binding fragment thereof, including, e.g., a VH, VL, scFv, light chain, or heavy chain (such as IgG1, IgG2, IgG4). Any of the known anti-CD19 antibodies may be used in the present invention, including, but not limited to, SAR3419, huBU12, loncastuximab, obexelimab, tafasitamab, taplitumomab, FMC63, SGN-19A, MDX-1342, SJ25C1, HD37, inebilizumab, GBR 401, B43, the anti-CD19 region of duvortuxizumab, biosimilars thereof, and derivatives thereof. Anti-CD19 antibodies have been described, for example, in U.S. Pat. No. 9,605,071, and International Publication Nos. WO2011/147834A1 and WO2017055328A1.

The term “CD3” is known in the art as a multi-protein complex of six chains (see, Abbas and Lichtman, 2003; Janeway et al., p172 and 178, 1999). In mammals, the complex comprises a CD3 gamma chain, a CD3 delta chain, two CD3 epsilon chains, and a homodimer of CD3 zeta chains. The CD3 gamma, CD3 delta, and CD3 epsilon chains are highly related cell surface proteins of the immunoglobulin superfamily containing a single immunoglobulin domain. The transmembrane regions of the CD3 gamma, CD3 delta, and CD3 epsilon chains are negatively charged, which is a characteristic that allows these chains to associate with the positively charged T cell receptor chains. The intracellular tails of the CD3 gamma, CD3 delta, and CD3 epsilon chains each contain a single conserved motif known as an immunoreceptor tyrosine-based activation motif or ITAM, whereas each CD3 zeta chain has three. Without being bound by theory, it is believed the ITAMs are important for the signaling capacity of a TCR complex. CD3 as used herein may be from various animal species, including human, primate, mouse, rat, or other mammals. For example, CD3 as used herein includes human CD3e (i.e., CD3 epsilon; e.g., UniProt accession number P07766), as well as variants, isoforms, and species homologs thereof (e.g., mouse CD3e (e.g., UniProt accession number P22646), rat CD3e (e.g., UniProt accession number A0A0G2K986), dog CD3e (e.g., UniProt accession number P27597), and cynomolgus monkey CD3e (e.g., UniProt accession number Q95LI5)).

The term “HER2” as used in the present application includes human HER2 (e.g., UniProt accession number P04626), as well as variants, isoforms, and species homologs thereof (e.g., mouse HER2 (UniProt accession number P70424), rat HER2 (UniProt accession number P06494), dog HER2, and cynomolgus monkey HER2). HER2 is also known as ERBB2.

The term “CD20” as used in the present application includes human CD20 (e.g., UniProt accession number P11836), as well as variants, isoforms, and species homologs thereof (e.g., mouse CD20 (e.g., UniProt accession number P19437), rat CD20, dog CD20, and cynomolgus monkey CD20). CD20 is also known as MS4A1.

The term “TROP2” as used in the present application includes human TROP2 (e.g., UniProt accession number P09758), as well as variants, isoforms, and species homologs thereof (e.g., mouse TROP2 (e.g., UniProt accession number Q8BGV3), rat TROP2 (e.g., UniProt accession number Q6P9Z6), dog TROP2, and cynomolgus monkey TROP2 (e.g., UniProt accession number A0A2K5UE71)). TROP2 is a transmembrane glycoprotein that transduces an intracellular calcium signal and acts as a cell surface receptor, and the upregulation of TROP2 is associated with cancer (see Zaman, S., et al., Onco Targets Ther. 2019; 12: 1781-1790; Goldenberg, D. M. et al., Oncotarget. 2018 Jun. 22; 9(48): 28989-29006). TROP2 is variously referred to as trophoblast cell-surface antigen 2 (TROP2), tumor associated calcium signal transducer 2, (TACSTD2), epithelial glycoprotein-1 (EGP1), GP50, membrane component surface marker-1 (M1S1), and gastrointestinal antigen 733-1 (GA7331).

The term “BCMA” as used in the present application includes human BCMA (e.g., UniProt accession number Q02223), as well as variants, isoforms, and species homologs thereof (e.g., mouse BCMA (e.g., UniProt accession number 088472), rat BCMA, dog BCMA, and cynomolgus monkey BCMA). BCMA is a cell surface receptor of the TNF receptor superfamily which recognizes B-cell activating factor (BAFF). BCMA is variously referred to as B-cell maturation antigen, BCM, tumor necrosis factor receptor superfamily member 17, TNFRSF17, CD269, and TNFRSF13A.

The term “CD19” as used in the present application includes human CD19 (e.g., UniProt accession number P15391), as well as variants, isoforms, and species homologs thereof (e.g., mouse CD19 (e.g., UniProt accession number P25918), rat CD19 (e.g., UniProt accession number F1LNH2), dog CD19 (e.g., UniProt accession number F1PJI6), and cynomolgus monkey CD19 (e.g., Uniprot accession number A0A2K5W8L9). CD19 is a B-lymphocyte antigen that is variously referred to as Cluster of Differentiation 19, B-Lymphocyte Surface Antigen B4, T-Cell Surface Antigen Leu-12, and CVID3.

The TBMs described herein may bind a human target (e.g., CD3, CD20, HER2 TROP2, BCMA, or CD19). In some cases, a TBM may be completely specific for the human target and may not exhibit species or other types of cross-reactivity. In other cases, a TBM also binds targets from species other than human. In some embodiments, the TBM is cross-reactive with the target molecule from at least one non-human species selected from the group consisting of cynomolgus monkey, mouse, rat and dog.

### i) Anti-CD3 Antibody

Also provided herein are isolated antibodies or antigen-binding fragments thereof that specifically bind to CD3 (e.g., human CD3). The anti-CD3 antibodies described herein may be used as in any one of the masked anti-CD3 antibodies, multispecific anti-CD3 antibodies, activatable anti-CD3 antibodies, and activatable multispecific T-cell engagers described herein.

In some embodiments, there is provided an isolated anti-CD3 antibody or antigen-binding fragment thereof comprising a) a VH comprising a CDR-H1 comprising the amino acid sequence according to Formula (I): X₁YAX₂X₃(SEQ ID NO: 382), wherein X₁ is D, S, or T, X₂ is I, L, or M, and X₃ is N or T, or a variant thereof comprising up to about 3 amino acid substitutions, a CDR-H2 comprising the amino acid sequence according to Formula (II): RIRSKYNNYATYYAX₁X₂VKX₃ (SEQ ID NO: 383), wherein X₁ is D or E, X₂ is S or T, and X₃ is D, G, or S, or a variant thereof comprising up to about 3 (5 (such as about any of 1, 2, or 3) amino acid substitutions, and a CDR-H3 comprising the amino acid sequence according to Formula (III): HGNX₁GX₂SYVSX₃X₄AY (SEQ ID NO: 384), wherein X₁ is F or Y, X₂ is N or T, X₃ is W or Y, and X₄ is F or W or a variant thereof comprising up to about 3 (such as about any of 1, 2, or 3) amino acid substitutions; and b) a VL comprising a CDR-L1 comprising the amino acid sequence according to Formula (IV): X₁SSTGAVTX₂X₃NYX₄N (SEQ ID NO: 385), wherein X₁ is A, G, or R, X₂ is S or T, X₃ is G or S, and X₄ is A, P, or V, or a variant thereof comprising up to about 3 (such as about any of 1, 2, or 3) amino acid substitutions, a CDR-L2 comprising the amino acid sequence according to Formula (V): GTX₁X₂RAP (SEQ ID NO: 386), wherein X₁ is K or N, and X₂ is F or K, or a variant thereof comprising up to about 3 (such as about any of 1, 2, or 3) amino acid substitutions, and a CDR-L3 comprising the amino acid sequence according to Formula (VI): ALWYSX₁X₂WV (SEQ ID NO: 387), wherein X₁ is D, N, or T, and X₂ is L or R, or a variant thereof comprising up to about 3 (such as about any of 1, 2, or 3) amino acid substitutions. In some embodiments, the amino acid substitutions are conservative amino acid substitutions.

In some embodiments, there is provided an isolated anti-CD3 antibody or antigen-binding fragment thereof, comprising a VH comprising a CDR-H1 comprising an amino acid sequence according to Formula (I): X₁YAX₂X₃(SEQ ID NO: 382), wherein X₁ is D, S, or T, X₂ is I, L, or M, and X₃ is N or T, a CDR-H2 comprising an amino acid sequence according to Formula (II): RIRSKYNNYATYYAX₁X₂VKX₃ (SEQ ID NO: 383), wherein X₁ is D or E, X₂ is S or T, and X₃ is D, G, or S, and a CDR-H3 comprising an amino acid sequence according to Formula (III): HGNX₁GX₂SYVSX₃X₄AY (SEQ ID NO: 384), wherein X₁ is F or Y, X₂ is N or T, X₃ is W or Y, and X₄ is F or W; and a VL comprising a CDR-L1 comprising an amino acid sequence according to Formula (IV): X₁SSTGAVTX₂X₃NYX₄N (SEQ ID NO: 385), wherein X₁ is A, G, or R, X₂ is S or T, X₃ is G or S, and X₄ is A, P, or V, a CDR-L2 comprising an amino acid sequence according to Formula (V): GTX₁X₂RAP (SEQ ID NO: 386), wherein X₁ is K or N, and X₂ is F or K, and a CDR-L3 comprising an amino acid sequence according to Formula (VI): ALWYSX₁X₂WV (SEQ ID NO: 387), wherein X₁ is D, N, or T, and X₂ is L or R.

In some embodiments, the anti-CD3 antibody or antigen-binding fragment thereof comprises a VH comprising a CDR-H1 comprising an amino acid sequence selected from the group consisting of SEQ ID NOs: 376 and 390, a CDR-H2 comprising an amino acid sequence selected from the group consisting of SEQ ID NOs: 391-394, and a CDR-H3 comprising an amino acid sequence selected from the group consisting of SEQ ID NOs: 378 and 395; and a VL comprising a CDR-L1 comprising an amino acid sequence selected from the group consisting of SEQ ID NOs: 396-398, a CDR-L2 comprising an amino acid sequence selected from the group consisting of SEQ ID NOs: 380 and 399, and a CDR-L3 comprising an amino acid sequence selected from the group consisting of SEQ ID NOs: 381, and 400-401.

In some embodiments, there is provided an isolated anti-CD3 antibody or antigen-binding fragment thereof comprising a VH comprising a CDR-H1 comprising an amino acid sequence selected from the group consisting of SEQ ID NOs: 376 and 390, or a variant thereof comprising up to about 3 (such as about any of 1, 2, or 3) amino acid substitutions; a CDR-H2 comprising an amino acid sequence selected from the group consisting of SEQ ID NOs: 391-394, or a variant thereof comprising up to about 3 (such as about any of 1, 2, or 3) amino acid substitutions, and a CDR-H3 comprising an amino acid sequence selected from the group consisting of SEQ ID NOs: 378 and 395, or a variant thereof comprising up to about 3 (such as about any of 1, 2, or 3) amino acid substitutions; and a VL comprising a CDR-L1 comprising an amino acid sequence selected from the group consisting of SEQ ID NOs: 396-398, or a variant thereof comprising up to about 3 (such as about any of 1, 2, or 3) amino acid substitutions, a CDR-L2 comprising an amino acid sequence selected from the group consisting of SEQ ID NOs: 380 and 399, or a variant thereof comprising up to about 3 (such as about any of 1, 2, or 3) amino acid substitutions, and a CDR-L3 comprising an amino acid sequence selected from the group consisting of SEQ ID NOs: 381, and 400-401, or a variant thereof comprising up to about 3 (such as about any of 1, 2, or 3) amino acid substitutions. In some embodiments, the amino acid substitution is a conservative amino acid substitution.

In some embodiments, there is provided an isolated anti-CD3 antibody or antigen-binding fragment thereof comprising a VH comprising a CDR-H1 comprising an amino acid sequence selected from the group consisting of SEQ ID NOs: 376 and 390, a CDR-H2 comprising an amino acid sequence selected from the group consisting of SEQ ID NOs: 391-394, and a CDR-H3 comprising an amino acid sequence selected from the group consisting of SEQ ID NOs: 378 and 395, or a variant thereof comprising up to about 3 (such as about any of 1, 2, or 3) amino acid substitutions in CDR-H1, CDR-H2 and/or CDR-H3; and a VL comprising a CDR-L1 comprising an amino acid sequence selected from the group consisting of SEQ ID NOs: 396-398, a CDR-L2 comprising an amino acid sequence selected from the group consisting of SEQ ID NOs: 380 and 399, and a CDR-L3 comprising an amino acid sequence selected from the group consisting of SEQ ID NOs: 381, and 400-401, or a variant thereof comprising up to about 3 (such as about any of 1, 2, or 3) amino acid substitutions in CDR-L1, CDR-L2 and/or CDR-L3. In some embodiments, the amino acid substitution is a conservative amino acid substitution.

In some embodiments, the anti-CD3 antibody or antigen-binding fragment thereof comprises 1, 2, 3, 4, 5, or 6 CDRs of an antibody as shown in Table 5D, Table 5E, and/or Table 5H. In some embodiments, the anti-CD3 antibody or antigen-binding fragment thereof comprises the VH of an antibody as shown in Table 5F. In some embodiments, the anti-CD3 antibody or antigen-binding fragment thereof comprises the VL of an antibody as shown in Table 5G. In some embodiments, the anti-CD3 antibody or antigen-binding fragment thereof comprises the VH and/or the VL of an antibody as shown in Table 5H. In some embodiments, the anti-CD3 antibody or antigen-binding fragment thereof comprises a VH comprising a CDR-H1 comprising an amino acid sequence selected from the group consisting of SEQ ID NOs: 376, 390, 601, and 602, a CDR-H2 comprising an amino acid sequence selected from the group consisting of SEQ ID NOs: 377, 391-394, and 603, and a CDR-H3 comprising an amino acid sequence selected from the group consisting of SEQ ID NOs: 378, 395, 604, and 605. In some embodiments, the anti-CD3 antibody or antigen-binding fragment thereof comprises a VL comprising a CDR-L1 comprising an amino acid sequence selected from the group consisting of SEQ ID NOs: 396-398, and 606-609, a CDR-L2 comprising an amino acid sequence selected from the group consisting of SEQ ID NOs: 380 and 399, and a CDR-L3 comprising an amino acid sequence selected from the group consisting of SEQ ID NOs: 381, 400-401, and 610. In some embodiments, the anti-CD3 antibody or antigen-binding fragment thereof comprises a VH comprising a CDR-H1 comprising an amino acid sequence selected from the group consisting of SEQ ID NOs: 376, 390, 601, and 602 or a variant thereof comprising up to about 3 (such as about any of 1, 2, or 3) amino acid substitutions, a CDR-H2 comprising an amino acid sequence selected from the group consisting of SEQ ID NOs: 377, 391-394, and 603 or a variant thereof comprising up to about 3 (such as about any of 1, 2, or 3) amino acid substitutions, and a CDR-H3 comprising an amino acid sequence selected from the group consisting of SEQ ID NOs: 378, 395, 604, and 605 or a variant thereof comprising up to about 3 (such as about any of 1, 2, or 3) amino acid substitutions. In some embodiments, the anti-CD3 antibody or antigen-binding fragment thereof comprises a VL comprising a CDR-L1 comprising an amino acid sequence selected from the group consisting of SEQ ID NOs: 396-398, and 606-609 or a variant thereof comprising up to about 3 (such as about any of 1, 2, or 3) amino acid substitutions, a CDR-L2 comprising an amino acid sequence selected from the group consisting of SEQ ID NOs: 380 and 399 or a variant thereof comprising up to about 3 (such as about any of 1, 2, or 3) amino acid substitutions, and a CDR-L3 comprising an amino acid sequence selected from the group consisting of SEQ ID NOs: 381, 400-401, and 610 or a variant thereof comprising up to about 3 (such as about any of 1, 2, or 3) amino acid substitutions.

In some embodiments, the anti-CD3 antibody or antigen-binding fragment thereof comprises a VH comprising an amino acid sequence selected from the group consisting of SEQ ID NOs: 67, 402, 405, 407, 409, 410, 412, 414-416, and 611-640. In some embodiments, the anti-CD3 antibody or antigen-binding fragment thereof comprises a VL comprising an amino acid sequence selected from the group consisting of SEQ ID NOs: 68, 403, 404, 406, 408, 411, 413, and 641-666.

In some embodiments, the anti-CD3 antibody or antigen-binding fragment thereof comprises a VH comprising an amino acid sequence comprising at least 80% (e.g., at least any one of 85%, 87%, 90%, 91%, 92%, 93%, 94%, 95%, 96%, 97%, 98%, 99%, or more) sequence identity to the group consisting of SEQ ID NOs: 67, 402, 405, 407, 409, 410, 412, 414-416, and 611-640. In some embodiments, the anti-CD3 antibody or antigen-binding fragment thereof comprises a VL comprising an amino acid sequence comprising at least 80% (e.g., at least any one of 85%, 87%, 90%, 91%, 92%, 93%, 94%, 95%, 96%, 97%, 98%, 99%, or more) sequence identity selected from the group consisting of SEQ ID NOs: 68, 403, 404, 406, 408, 411, 413, and 641-666.

In some embodiments, the anti-CD3 antibody or antigen-binding fragment thereof comprises a VH comprising a CDR-H1 comprising the amino acid sequence of SEQ ID NO: 376, a CDR-H2 comprising the amino acid sequence of SEQ ID NO: 391, and a CDR-H3 comprising the amino acid sequence of SEQ ID NO: 378, and a VL comprising a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381.

In some embodiments, the anti-CD3 antibody or antigen-binding fragment thereof comprises a VH comprising a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395, and a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400.

In some embodiments, the anti-CD3 antibody or antigen-binding fragment thereof comprises a VH comprising a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395, and a VL comprising a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 401.

In some embodiments, the anti-CD3 antibody or antigen-binding fragment thereof comprises a VH comprising a CDR-H1 comprising the amino acid sequence of SEQ ID NO: 390, a CDR-H2 comprising the amino acid sequence of SEQ ID NO: 393, and a CDR-H3 comprising the amino acid sequence of SEQ ID NO: 395, and a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381.

In some embodiments, the anti-CD3 antibody or antigen-binding fragment thereof comprises a VH comprising a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395, and a VL comprising a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 401.

In some embodiments, the anti-CD3 antibody or antigen-binding fragment thereof comprises a VH comprising a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395, and a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400.

In some embodiments, the anti-CD3 antibody or antigen-binding fragment thereof comprises a VH comprising a CDR-H1 comprising the amino acid sequence of SEQ ID NO: 376, a CDR-H2 comprising the amino acid sequence of SEQ ID NO: 393, and a CDR-H3 comprising the amino acid sequence of SEQ ID NO: 395, and a VL comprising a CDR-L1 comprising the amino acid sequence of SEQ ID NO: 398, a CDR-L2 comprising the amino acid sequence of SEQ ID NO: 399, and a CDR-L3 comprising the amino acid sequence of SEQ ID NO: 400.

In some embodiments, the anti-CD3 antibody or antigen-binding fragment thereof comprises a VH comprising a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395, and a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381.

In some embodiments, the anti-CD3 antibody or antigen-binding fragment thereof comprises a VH comprising a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 395, and a VL comprising a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381.

In some embodiments, the anti-CD3 antibody or antigen-binding fragment thereof comprises a VH comprising a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395, and a VL comprising a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381.

In some embodiments, the anti-CD3 antibody or antigen-binding fragment thereof comprises a VH comprising a CDR-H1 comprising the amino acid sequence of SEQ ID NO: 376, a CDR-H2 comprising the amino acid sequence of SEQ ID NO: 391, and a CDR-H3 comprising the amino acid sequence of SEQ ID NO: 378, and a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400.

In some embodiments, the anti-CD3 antibody or antigen-binding fragment thereof comprises a VH comprising a CDR-H1 comprising the amino acid sequence of SEQ ID NO: 390, a CDR-H2 comprising the amino acid sequence of SEQ ID NO: 393, and a CDR-H3 comprising the amino acid sequence of SEQ ID NO: 378, and a VL comprising a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381.

In some embodiments, the anti-CD3 antibody or antigen-binding fragment thereof comprises a VH comprising a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 378, and a VL comprising a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 381.

In some embodiments, the anti-CD3 antibody or antigen-binding fragment thereof comprises a VH comprising a CDR-H1 comprising the amino acid sequence of SEQ ID NO: 390, a CDR-H2 comprising the amino acid sequence of SEQ ID NO: 391, and a CDR-H3 comprising the amino acid sequence of SEQ ID NO: 378, and a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381.

In some embodiments, the anti-CD3 antibody or antigen-binding fragment thereof comprises a VH comprising a CDR-H1 comprising the amino acid sequence of SEQ ID NO: 688, a CDR-H2 comprising the amino acid sequence of SEQ ID NO: 391, and a CDR-H3 comprising the amino acid sequence of SEQ ID NO: 689, and a VL comprising a CDR-L1 comprising the amino acid sequence of SEQ ID NO: 398, a CDR-L2 comprising the amino acid sequence of SEQ ID NO: 399, and a CDR-L3 comprising the amino acid sequence of SEQ ID NO: 687.

In some embodiments, the anti-CD3 antibody or antigen-binding fragment thereof comprises a VH comprising a CDR-H1 comprising the amino acid sequence of SEQ ID NO: 688, a CDR-H2 comprising the amino acid sequence of SEQ ID NO: 377, and a CDR-H3 comprising the amino acid sequence of SEQ ID NO: 689, and a VL comprising a CDR-L1 comprising the amino acid sequence of SEQ ID NO: 690, a CDR-L2 comprising the amino acid sequence of SEQ ID NO: 399, and a CDR-L3 comprising the amino acid sequence of SEQ ID NO: 687.

In some embodiments, the anti-CD3 antibody or antigen-binding fragment thereof comprises a VH comprising a CDR-H1 comprising the amino acid sequence of SEQ ID NO: 688, a CDR-H2 comprising the amino acid sequence of SEQ ID NO: 394, and a CDR-H3 comprising the amino acid sequence of SEQ ID NO: 692, and a VL comprising a CDR-L1 comprising the amino acid sequence of SEQ ID NO: 396, a CDR-L2 comprising the amino acid sequence of SEQ ID NO: 380, and a CDR-L3 comprising the amino acid sequence of SEQ ID NO: 691.

In some embodiments, the anti-CD3 antibody or antigen-binding fragment thereof comprises a VH comprising a CDR-H1 comprising the amino acid sequence of SEQ ID NO: 396, a CDR-H2 comprising the amino acid sequence of SEQ ID NO: 399, and a CDR-H3 comprising the amino acid sequence of SEQ ID NO: 687, and a VL comprising a CDR-L1 comprising the amino acid sequence of SEQ ID NO: 390, a CDR-L2 comprising the amino acid sequence of SEQ ID NO: 394, and a CDR-L3 comprising the amino acid sequence of SEQ ID NO: 395.

In some embodiments, the anti-CD3 antibody or antigen-binding fragment thereof comprises a VH comprising a CDR-H1 comprising the amino acid sequence of SEQ ID NO: 397, a CDR-H2 comprising the amino acid sequence of SEQ ID NO: 380, and a CDR-H3 comprising the amino acid sequence of SEQ ID NO: 381, and a VL comprising a CDR-L1 comprising the amino acid sequence of SEQ ID NO: 688, a CDR-L2 comprising the amino acid sequence of SEQ ID NO: 394, and a CDR-L3 comprising the amino acid sequence of SEQ ID NO: 604.

In some embodiments, the anti-CD3 antibody or antigen-binding fragment thereof comprises a VH comprising a CDR-H1 comprising the amino acid sequence of SEQ ID NO: 397, a CDR-H2 comprising the amino acid sequence of SEQ ID NO: 399, and a CDR-H3 comprising the amino acid sequence of SEQ ID NO: 691, and a VL comprising a CDR-L1 comprising the amino acid sequence of SEQ ID NO: 390, a CDR-L2 comprising the amino acid sequence of SEQ ID NO: 394, and a CDR-L3 comprising the amino acid sequence of SEQ ID NO: 689.

In some embodiments, the anti-CD3 antibody or antigen-binding fragment thereof comprises a VH comprising a CDR-H1 comprising the amino acid sequence of SEQ ID NO: 396, a CDR-H2 comprising the amino acid sequence of SEQ ID NO: 399, and a CDR-H3 comprising the amino acid sequence of SEQ ID NO: 691, and a VL comprising a CDR-L1 comprising the amino acid sequence of SEQ ID NO: 390, a CDR-L2 comprising the amino acid sequence of SEQ ID NO: 391, and a CDR-L3 comprising the amino acid sequence of SEQ ID NO: 604.

In some embodiments, the anti-CD3 antibody or antigen-binding fragment thereof comprises a VH comprising a CDR-H1 comprising the amino acid sequence of SEQ ID NO: 397, a CDR-H2 comprising the amino acid sequence of SEQ ID NO: 399, and a CDR-H3 comprising the amino acid sequence of SEQ ID NO: 687, and a VL comprising a CDR-L1 comprising the amino acid sequence of SEQ ID NO: 688, a CDR-L2 comprising the amino acid sequence of SEQ ID NO: 393, and a CDR-L3 comprising the amino acid sequence of SEQ ID NO: 604.

In some embodiments, the anti-CD3 antibody or antigen-binding fragment thereof comprises a VH comprising a CDR-H1 comprising the amino acid sequence of SEQ ID NO: 396, a CDR-H2 comprising the amino acid sequence of SEQ ID NO: 380, and a CDR-H3 comprising the amino acid sequence of SEQ ID NO: 400, and a VL comprising a CDR-L1 comprising the amino acid sequence of SEQ ID NO: 688, a CDR-L2 comprising the amino acid sequence of SEQ ID NO: 693, and a CDR-L3 comprising the amino acid sequence of SEQ ID NO: 692.

In some embodiments, the anti-CD3 antibody or antigen-binding fragment thereof comprises a VH comprising a CDR-H1 comprising the amino acid sequence of SEQ ID NO: 694, a CDR-H2 comprising the amino acid sequence of SEQ ID NO: 399, and a CDR-H3 comprising the amino acid sequence of SEQ ID NO: 691, and a VL comprising a CDR-L1 comprising the amino acid sequence of SEQ ID NO: 695, a CDR-L2 comprising the amino acid sequence of SEQ ID NO: 696, and a CDR-L3 comprising the amino acid sequence of SEQ ID NO: 604.

In some embodiments, the anti-CD3 antibody or antigen-binding fragment thereof comprises a VH comprising an amino acid sequence according to Formula (VII): EVQLVESGGGLVX₁PGGSLRLSCAASGFTFX₂X₃YAIX₄WVRQAPGKGLEWVX₅RIRSKYN NYATYYAX₆SVKX₇RFTISRDX₈SKNTLYLQX₉NSLRAEDTAVYYCX₁₀RHGNX₁₁GX₁₂SYVS WFAYWGQGTLVTVSS (SEQ ID NO: 388), wherein X₁ is K or Q, X₂ is N or S, X₃ is S or T, X₄ is H or N, X₅ is G or S, X₆ is D or E, X₇ is D or G, X₈ is D or N, X₉ is I or L, X₁₀ is A or V, X₁₁ is F or Y, X₁₂ is N or T; and a VL comprising an amino acid sequence according to Formula (VIII): X₁AVVTQEPSLTVSPGGTVTLTCX₂SSTGAVTTSNYX₃NWX₄QQKPGQAPRGLIGGTX₅X₆R APGX₇PARFSGSLLGGKAALTLSGAQPEDEAEYYCALWYSX₈X₉WVFGGGTKLTVL (SEQ ID NO: 389), wherein X₁ is E or Q, X₂ is A, G, P, or R, X₃ is A or P, X₄ is F or V, X₅ is K or N, X₆ is F or K, X₇ is A, I, T, or V, X₈ is A, D, N, or T, and X₉ is H or L.

In some embodiments, the anti-CD3 antibody or antigen-binding fragment thereof comprises a VH comprising an amino acid sequence selected from the group consisting of SEQ ID NOs: 67, 402, 405, 407, 409, 410, 412, 414, 415, and 416; and a VL comprising an amino acid sequence selected from the group consisting of SEQ ID NOs: 69, 403, 404, 406, 408, 411, and 413.

In some embodiments, the anti-CD3 antibody or antigen-binding fragment thereof comprises a VH comprising the amino acid sequence of SEQ ID NO: 67, and a VL comprising the amino acid sequence of SEQ ID NO: 68.

In some embodiments, the anti-CD3 antibody or antigen-binding fragment thereof comprises a VH comprising the amino acid sequence of SEQ ID NO: 402, and a VL comprising the amino acid sequence of SEQ ID NO: 403.

In some embodiments, the anti-CD3 antibody or antigen-binding fragment thereof comprises a VH comprising the amino acid sequence of SEQ ID NO: 402, and a VL comprising the amino acid sequence of SEQ ID NO: 403.

In some embodiments, the anti-CD3 antibody or antigen-binding fragment thereof comprises a VH comprising the amino acid sequence of SEQ ID NO: 402, and a VL comprising the amino acid sequence of SEQ ID NO: 404.

In some embodiments, the anti-CD3 antibody or antigen-binding fragment thereof comprises a VH comprising the amino acid sequence of SEQ ID NO: 405, and a VL comprising the amino acid sequence of SEQ ID NO: 406.

In some embodiments, the anti-CD3 antibody or antigen-binding fragment thereof comprises a VH comprising the amino acid sequence of SEQ ID NO: 407, and a VL comprising the amino acid sequence of SEQ ID NO: 404.

In some embodiments, the anti-CD3 antibody or antigen-binding fragment thereof comprises a VH comprising the amino acid sequence of SEQ ID NO: 407, and a VL comprising the amino acid sequence of SEQ ID NO: 403.

In some embodiments, the anti-CD3 antibody or antigen-binding fragment thereof comprises a VH comprising the amino acid sequence of SEQ ID NO: 407, and a VL comprising the amino acid sequence of SEQ ID NO: 408.

In some embodiments, the anti-CD3 antibody or antigen-binding fragment thereof comprises a VH comprising the amino acid sequence of SEQ ID NO: 409, and a VL comprising the amino acid sequence of SEQ ID NO: 408.

In some embodiments, the anti-CD3 antibody or antigen-binding fragment thereof comprises a VH comprising the amino acid sequence of SEQ ID NO: 410, and a VL comprising the amino acid sequence of SEQ ID NO: 411.

In some embodiments, the anti-CD3 antibody or antigen-binding fragment thereof comprises a VH comprising the amino acid sequence of SEQ ID NO: 412, and a VL comprising the amino acid sequence of SEQ ID NO: 413.

In some embodiments, the anti-CD3 antibody or antigen-binding fragment thereof comprises a VH comprising the amino acid sequence of SEQ ID NO: 410, and a VL comprising the amino acid sequence of SEQ ID NO: 413.

In some embodiments, the anti-CD3 antibody or antigen-binding fragment thereof comprises a VH comprising the amino acid sequence of SEQ ID NO: 414, and a VL comprising the amino acid sequence of SEQ ID NO: 403.

In some embodiments, the anti-CD3 antibody or antigen-binding fragment thereof comprises a VH comprising the amino acid sequence of SEQ ID NO: 415, and a VL comprising the amino acid sequence of SEQ ID NO: 413.

In some embodiments, the anti-CD3 antibody or antigen-binding fragment thereof comprises a VH comprising the amino acid sequence of SEQ ID NO: 416, and a VL comprising the amino acid sequence of SEQ ID NO: 413.

In some embodiments, the anti-CD3 antibody or antigen-binding fragment thereof comprises a VH comprising the amino acid sequence of SEQ ID NO: 416, and a VL comprising the amino acid sequence of SEQ ID NO: 411.

In some embodiments, the isolated antibody or antigen-binding fragment thereof that specifically binds CD3 comprises one, two, three, four, five, or six CDRs of an antibody as shown in Table 7. In some embodiments, the isolated antibody or antigen-binding fragment thereof that specifically binds CD3 comprises one, two, three, four, five, or six CDRs of the anti-CD3 antibody TY24051, TY25236, TY25023, TY25024, TY25237, TY25228, TY25227, TY25230, TY25229, TY25238, TY25239, TY25243, TY25231, TY25244, TY25241, or TY25240, as shown in Table 7. In some embodiments, the isolated antibody or antigen-binding fragment thereof that specifically binds CD3 comprises a VH and/or a VL as shown in Table 8. In some embodiments, the isolated antibody or antigen-binding fragment thereof that specifically binds CD3 comprises a VH and/or a VL of the anti-CD3 antibody TY24051, TY25236, TY25023, TY25024, TY25237, TY25228, TY25227, TY25230, TY25229, TY25238, TY25239, TY25243, TY25231, TY25244, TY25241, or TY25240, as shown in Table 8.

In some embodiments, the isolated antibody or antigen-binding fragment thereof that specifically binds CD3 comprises one, two, three, four, five, or six CDRs of antibody TY25023 as shown in Table 7. In some embodiments, the antibody or antigen-binding fragment thereof comprises a VH and/or a VL of antibody TY25023 as shown in Table 8. In some embodiments, the antibody or antigen-binding fragment thereof comprises a scFv of antibody TY25023 as shown in Table 9. In some embodiments, the antibody or antigen-binding fragment thereof comprises a heavy chain of antibody TY25023 as shown in Table 12.

In some embodiments, the isolated antibody or antigen-binding fragment thereof that specifically binds CD3 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:402. In certain embodiments, a VH sequence contains substitutions (e.g., conservative substitutions), insertions, or deletions relative to the amino acid sequence of SEQ ID NO:402, but retains the same ability to bind CD3 as the antibody comprising SEQ ID NO: 402. In certain embodiments, a total of 1 to 13 amino acids have been substituted, inserted and/or deleted in SEQ ID NO: 402. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO:390, (b) a CDR-H2 comprising the amino acid sequence of SEQ ID NO:392, and (c) a CDR-H3 comprising the amino acid sequence of SEQ ID NO:395.

In another aspect, an isolated antibody or antigen-binding fragment thereof that specifically binds CD3 is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO:403. In certain embodiments, a VL sequence contains substitutions (e.g., conservative substitutions), insertions, or deletions relative to the amino acid sequence of SEQ ID NO:403, but retains the same ability to bind CD3 as the antibody comprising SEQ ID NO:403. In certain embodiments, a total of 1 to 11 amino acids have been substituted, inserted and/or deleted in SEQ ID NO: 403. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO:397; (b) a CDR-L2 comprising the amino acid sequence of SEQ ID NO:380; and (c) a CDR-L3 comprising the amino acid sequence of SEQ ID NO:400.

In some embodiments, the isolated antibody or antigen-binding fragment thereof that specifically binds CD3 comprises a VH comprising a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395, and a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 400.

In some embodiments, the isolated antibody or antigen-binding fragment thereof that specifically binds CD3 comprises a VH comprising the amino acid sequence of SEQ ID NO: 402, and the VL comprising the amino acid sequence of SEQ ID NO: 403.

In some embodiments, the isolated antibody or antigen-binding fragment thereof that specifically binds CD3 comprises a VH CDR1, a VH CDR2, and a VH CDR3, respectively comprising the amino acid sequences of a CDR1, a CDR2, and a CDR3 of a VH having the sequence set forth in SEQ ID NO:402; and a VL CDR1, a VL CDR2, and a VL CDR3, respectively comprising the amino acid sequences of a CDR1, a CDR2, and a CDR3 of a VL having the sequence set forth in SEQ ID NO:403.

In some embodiments, the isolated antibody or antigen-binding fragment thereof that specifically binds CD3 comprises one, two, three, four, five, or six CDRs of antibody TY25238 as shown in Table 7. In some embodiments, the antibody or antigen-binding fragment thereof comprises a VH and/or a VL of antibody TY25238 as shown in Table 8. In some embodiments, the antibody or antigen-binding fragment thereof comprises a scFv of antibody TY25238 as shown in Table 9. In some embodiments, the antibody or antigen-binding fragment thereof comprises a heavy chain of antibody TY25238 as shown in Table 12.

In some embodiments, the isolated antibody or antigen-binding fragment thereof that specifically binds CD3 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:410. In certain embodiments, a VH sequence contains substitutions (e.g., conservative substitutions), insertions, or deletions relative to the amino acid sequence of SEQ ID NO: 410, but retains the same ability to bind CD3 as the antibody comprising SEQ ID NO: 410. In certain embodiments, a total of 1 to 13 amino acids have been substituted, inserted and/or deleted in SEQ ID NO: 410. In certain embodiments, substitutions, insertions, or deletions occur in regions outside the CDRs (i.e., in the FRs). In a particular embodiment, the VH comprises one, two or three CDRs selected from the group consisting of: (a) a CDR-H1 comprising the amino acid sequence of SEQ ID NO:390, (b) a CDR-H2 comprising the amino acid sequence of SEQ ID NO:394, and (c) a CDR-H3 comprising the amino acid sequence of SEQ ID NO:395.

In another aspect, an isolated antibody or antigen-binding fragment thereof that specifically binds CD3 is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO:411. In certain embodiments, a VL sequence contains substitutions (e.g., conservative substitutions), insertions, or deletions relative to the amino acid sequence of SEQ ID NO: 411, but retains the same ability to bind CD3 as the antibody comprising SEQ ID NO: 411. In certain embodiments, a total of 1 to 11 amino acids have been substituted, inserted and/or deleted in SEQ ID NO: 411. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO:397; (b) a CDR-L2 comprising the amino acid sequence of SEQ ID NO:380; and (c) a CDR-L3 comprising the amino acid sequence of SEQ ID NO:381.

In some embodiments, the isolated antibody or antigen-binding fragment thereof that specifically binds CD3 comprises a VH comprising a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395, and a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381.

In some embodiments, the isolated antibody or antigen-binding fragment thereof that specifically binds CD3 comprises a VH comprising the amino acid sequence of SEQ ID NO: 410, and a VL comprising the amino acid sequence of SEQ ID NO: 411.

In some embodiments, the isolated antibody or antigen-binding fragment thereof that specifically binds CD3 comprises a VH CDR1, a VH CDR2, and a VH CDR3, respectively comprising the amino acid sequences of a CDR1, a CDR2, and a CDR3 of a VH having the sequence set forth in SEQ ID NO:410; and a VL CDR1, a VL CDR2, and a VL CDR3, respectively comprising the amino acid sequences of a CDR1, a CDR2, and a CDR3 of a VL having the sequence set forth in SEQ ID NO:411.

In some embodiments, the isolated antibody or antigen-binding fragment thereof that specifically binds CD3 is a Fab, a Fv, or a scFv. In some embodiments, the isolated antibody or antigen-binding fragment thereof that specifically binds CD3 is a scFv comprising from the N-terminus to the C-terminus: VL-L1-VH, wherein L1 is a peptide linker. In some embodiments, the TBM is a scFv comprising from the N-terminus to the C-terminus: VH-L1-VL, wherein L1 is a peptide linker. In some embodiment, L1 comprises the amino acid sequence of SEQ ID NO: 82.

In some embodiments, the isolated antibody or antigen-binding fragment thereof that specifically binds CD3 comprises 1, 2, 3, 4, 5, or 6 CDRs as shown in Table 5B, Table 5D, Table 5E, and/or Table 5H. In some embodiments, the isolated antibody or antigen-binding fragment thereof that specifically binds CD3 comprises a VH and/or VL as shown in Table 5C, Table 5F, Table 5G, and/or Table 5H. In some embodiments, the isolated antibody or antigen-binding fragment thereof that specifically binds CD3 comprises a VH and/or a VL of the anti-CD3 antibody TY25520, TY25521, TY25523, TY25524, TY25525, TY25526, TY25527, TY25528, TY25529, or TY25531, as shown in Table 5H.

In some embodiments, the isolated antibody or antigen-binding fragment thereof that specifically binds CD3 binds human CD3. In some embodiments, the isolated antibody, or antigen-binding fragment thereof, is cross-reactive with a CD3 polypeptide from at least one non-human species selected from the group consisting of cynomolgus monkey, mouse, rat and dog.

In some embodiments, the isolated anti-CD3 antibody is a monospecific antibody, e.g., a full-length antibody, a scFv, or a scFv-Fc. In some embodiments, the isolated anti-CD3 antibody is a multispecific (e.g., bispecific) T-cell engager. In some embodiments, the isolated anti-CD3 antibody is an activatable antibody. In some embodiments, the isolated anti-CD3 antibody is an activatable multispecific antibody, such as an activatable multispecific (e.g., bispecific) T-cell engager.

### ii) Anti-CD20 Antibody

Also provided herein are isolated antibodies or antigen-binding fragments thereof that specifically bind to CD20 (e.g., human CD20). The anti-CD20 antibodies described herein may be used as in any one of the multispecific anti-CD20 antibodies, activatable anti-CD20 antibodies, and activatable multispecific T-cell engagers described herein.

In some embodiments, the isolated antibody or antigen-binding fragment thereof that specifically binds CD20 comprises 1, 2, 3, 4, 5, or 6 CDRs as shown in Tables C, 29 and 31. In some embodiments, the isolated antibody or antigen-binding fragment thereof that specifically binds CD20 comprises a VH and/or VL as shown in Tables C, 29 and 31.

In some embodiments, the anti-CD20 antibody or antigen-binding fragment thereof comprises a VH comprising a CDR-H1 comprising the amino acid sequence of SEQ ID NO: 556, a CDR-H2 comprising the amino acid sequence of SEQ ID NO: 557, and a CDR-H3 comprising the amino acid sequence of SEQ ID NO: 558, and a VL comprising a CDR-L1 comprising the amino acid sequence of SEQ ID NO: 559, a CDR-L2 comprising the amino acid sequence of SEQ ID NO: 560, and a CDR-L3 comprising the amino acid sequence of SEQ ID NO: 561.

In some embodiments, the anti-CD20 antibody or antigen-binding fragment thereof comprises a VH comprising a CDR-H1 comprising the amino acid sequence of SEQ ID NO: 86, a CDR-H2 comprising the amino acid sequence of SEQ ID NO: 557, and a CDR-H3 comprising the amino acid sequence of SEQ ID NO: 558, and a VL comprising a CDR-L1 comprising the amino acid sequence of SEQ ID NO: 559, a CDR-L2 comprising the amino acid sequence of SEQ ID NO: 560, and a CDR-L3 comprising the amino acid sequence of SEQ ID NO: 561.

In some embodiments, the anti-CD20 antibody or antigen-binding fragment thereof comprises a VH comprising a CDR-H1 comprising the amino acid sequence of SEQ ID NO: 556 or 86 or a variant thereof comprising up to about 3 (such as about any of 1, 2, or 3) amino acid substitutions, a CDR-H2 comprising the amino acid sequence of SEQ ID NO: 557 or a variant thereof comprising up to about 3 (such as about any of 1, 2, or 3) amino acid substitutions, and a CDR-H3 comprising the amino acid sequence of SEQ ID NO: 558 or a variant thereof comprising up to about 3 (such as about any of 1, 2, or 3) amino acid substitutions, and a VL comprising a CDR-L1 comprising the amino acid sequence of SEQ ID NO: 559 or a variant thereof comprising up to about 3 (such as about any of 1, 2, or 3) amino acid substitutions, a CDR-L2 comprising the amino acid sequence of SEQ ID NO: 560 or a variant thereof comprising up to about 3 (such as about any of 1, 2, or 3) amino acid substitutions, and a CDR-L3 comprising the amino acid sequence of SEQ ID NO: 561 or a variant thereof comprising up to about 3 (such as about any of 1, 2, or 3) amino acid substitutions. In some embodiments, the amino acid substitution is a conservative amino acid substitution.

In some embodiments, the isolated antibody or antigen-binding fragment thereof that specifically binds human CD20 comprises a VH CDR1, a VH CDR2, and a VH CDR3, respectively comprising the amino acid sequences of a CDR1, a CDR2, and a CDR3 of a VH having the sequence set forth in SEQ ID NO:562; and a VL CDR1, a VL CDR2, and a VL CDR3, respectively comprising the amino acid sequences of a CDR1, a CDR2, and a CDR3 of a VL having the sequence set forth in SEQ ID NO:563. In some embodiments, the VH CDR1, VH CDR2, VH CDR3, VL CDR1, VL CDR2, and/or VL CDR3 comprises up to about 3 (such as about any of 1, 2, or 3) amino acid substitutions. In some embodiments, the amino acid substitution is a conservative amino acid substitution.

In some embodiments, the isolated antibody or antigen-binding fragment thereof that specifically binds CD20 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:562. In some embodiments, the VH comprises an amino acid sequence comprising at least 80% (e.g., at least any one of 85%, 87%, 90%, 91%, 92%, 93%, 94%, 95%, 96%, 97%, 98%, 99%, or more) sequence identity to the amino acid sequence of SEQ ID NO:562. In certain embodiments, a VH sequence contains substitutions (e.g., conservative substitutions), insertions, or deletions relative to the amino acid sequence of SEQ ID NO: 562, but retains the same ability to bind CD20 as the antibody comprising SEQ ID NO: 562. In certain embodiments, a total of 1 to 13 amino acids have been substituted, inserted and/or deleted in SEQ ID NO: 562. In certain embodiments, substitutions, insertions, or deletions occur in regions outside the CDRs (i.e., in the FRs). In some embodiments, the VH comprises one, two or three CDRs selected from the group consisting of: (a) a CDR-H1 comprising the amino acid sequence of SEQ ID NO:556, (b) a CDR-H2 comprising the amino acid sequence of SEQ ID NO:557, and (c) a CDR-H3 comprising the amino acid sequence of SEQ ID NO:558. In some embodiments, the VH comprises one, two or three CDRs selected from the group consisting of: (a) a CDR-H1 comprising the amino acid sequence of SEQ ID NO:86, (b) a CDR-H2 comprising the amino acid sequence of SEQ ID NO:557, and (c) a CDR-H3 comprising the amino acid sequence of SEQ ID NO:558.

In another aspect, an isolated antibody or antigen-binding fragment thereof that specifically binds CD3 is provided, wherein the antibody or antigen-binding fragment thereof comprises a light chain variable domain (VL) having at least 90%, 91%, 92%, 93%, 94%, 95%, 96%, 97%, 98%, 99%, or 100% sequence identity to the amino acid sequence of SEQ ID NO:563. In some embodiments, the VL comprises an amino acid sequence comprising at least 80% (e.g., at least any one of 85%, 87%, 90%, 91%, 92%, 93%, 94%, 95%, 96%, 97%, 98%, 99%, or more) sequence identity to the amino acid sequence of SEQ ID NO:563. In certain embodiments, a VL sequence contains substitutions (e.g., conservative substitutions), insertions, or deletions relative to the amino acid sequence of SEQ ID NO: 563, but retains the same ability to bind CD20 as the antibody comprising SEQ ID NO: 563. In certain embodiments, a total of 1 to 11 amino acids have been substituted, inserted and/or deleted in SEQ ID NO: 563. In certain embodiments, the substitutions, insertions, or deletions occur in regions outside the CDRs (i.e., in the FRs). In a particular embodiment, the VL comprises one, two or three CDRs selected from the group consisting of (a) a CDR-L1 comprising the amino acid sequence of SEQ ID NO:559; (b) a CDR-L2 comprising the amino acid sequence of SEQ ID NO:560; and (c) a CDR-L3 comprising the amino acid sequence of SEQ ID NO:561.

In some embodiments, the anti-CD20 antibody or antigen-binding fragment thereof comprises a VH comprising the amino acid sequence of SEQ ID NO: 562, and a VL comprising the amino acid sequence of SEQ ID NO: 563.

In some embodiments, the anti-CD20 antibody or antigen-binding fragment thereof comprises a VH comprising the amino acid sequence of SEQ ID NO: 87, and a VL comprising the amino acid sequence of SEQ ID NO: 89.

In some embodiments, the anti-CD20 antibody or antigen-binding fragment thereof comprises a VH comprising the amino acid sequence of SEQ ID NO: 87, and a VL comprising the amino acid sequence of SEQ ID NO: 90.

In some embodiments, the anti-CD20 antibody or antigen-binding fragment thereof comprises a VH comprising the amino acid sequence of SEQ ID NO: 87, and a VL comprising the amino acid sequence of SEQ ID NO: 91.

In some embodiments, the isolated antibody or antigen-binding fragment thereof that specifically binds CD20 is a Fab, a Fv, or a scFv.

In some embodiments, the isolated antibody or antigen-binding fragment thereof that specifically binds CD20 comprises a light chain (e.g., an LC1) of antibody TY25455, TY25023, or TY25238, as shown in Table 23. In some embodiments, the isolated antibody or antigen-binding fragment thereof that specifically binds CD20 comprises a light chain comprising the amino acid sequence of SEQ ID NO: 564.

In some embodiments, the isolated antibody or antigen-binding fragment thereof that specifically binds CD20 comprises a heavy chain (e.g., an HC1) of antibody TY25455, TY25023, or TY25238, as shown in Table 23. In some embodiments, the isolated antibody or antigen-binding fragment thereof that specifically binds CD20 comprises a light chain comprising the amino acid sequence of SEQ ID NO: 565.

In some embodiments, the isolated antibody or antigen-binding fragment thereof that specifically binds CD20 is more highly expressed than a reference antibody (e.g., rituximab). In some embodiments, the isolated antibody or antigen-binding fragment thereof that specifically binds CD20 is produced to a higher abundance of protein than a reference antibody (e.g., rituximab). In some embodiments, the isolated antibody or antigen-binding fragment thereof that specifically binds CD20 is more likely to be properly folded than a reference antibody (e.g., rituximab). In some embodiments, the expression, protein abundance, or level of proper folding compared to a reference antibody (e.g., rituximab) is measured under controlled experimental conditions. In some embodiments, the expression, protein abundance, or level of proper folding compared to a reference antibody (e.g., rituximab) is measured with the isolated antibody or antigen-binding fragment thereof that specifically binds CD20 and the reference antibody in an activatable and/or multispecific format, as described herein.

In some embodiments, the isolated anti-CD20 antibody is a monospecific antibody, e.g., a full-length antibody, a scFv, or a scFv-Fc. In some embodiments, the isolated anti-CD20 antibody is a multispecific (e.g., bispecific) T-cell engager. In some embodiments, the isolated anti-CD20 antibody is an activatable antibody. In some embodiments, the isolated anti-CD20 antibody is an activatable multispecific antibody, such as an activatable multispecific (e.g., bispecific) T-cell engager.

### I. Linker

The antibodies (e.g., multispecific antibodies, activatable multispecific antibodies, activatable anti-CD3 antibodies, masked anti-CD3 antibodies, and activatable anti-HER2 antibodies) described herein may comprise one or more linkers (e.g., L1, L2, L3, etc.) disposed between the various regions in the polypeptides.

Any suitable linker (e.g., a flexible linker) known in the art may be used, including, for example: glycine polymers (G)n, where n is an integer of at least 1 (e.g., at least one, at least 2, at least 3, at least 4, at least 5, at least 6, at least 7, at least 8, at least 9, at least 10, etc.); glycine-serine polymers (GS)n, where n is an integer of at least 1 (e.g., at least one, at least 2, at least 3, at least 4, at least 5, at least 6, at least 7, at least 8, at least 9, at least 10, etc.) such as SGGGS (SEQ ID NO: 80), GGGSGGGGS (SEQ ID NO: 81), (G₄S)₄ (SEQ ID NO: 82), GGGGS (SEQ ID NO: 130), SGGS (SEQ ID NO: 131), GGSG (SEQ ID NO: 132), GGSGG (SEQ ID NO: 133), GSGSG (SEQ ID NO: 134), GSGGG (SEQ ID NO: 135), GGGSG (SEQ ID NO: 136), and/or GSSSG (SEQ ID NO: 137)); glycine-alanine polymers; alanine-serine polymers; and the like. Linker sequences may be of any length, such as from about 1 amino acid (e.g., glycine or serine) to about 20 amino acids (e.g., 20 amino acid glycine polymers or glycine-serine polymers), about 1 amino acid to about 15 amino acids, about 3 amino acids to about 12 amino acids, about 4 amino acids to about 10 amino acids, about 5 amino acids to about 9 amino acids, about 6 amino acids to about 8 amino acids, etc. In some embodiments, the linker is any of about 1, 2, 3, 4, 5, 6, 7, 8, 9, 10, 11, 12, 13, 14, 15, 16, 17, 18, 19, or 20 amino acids in length.

### J. Fc Regions and CH3 Domains

In some embodiments, the antibody (e.g., multispecific antibody, activatable multispecific antibody, activatable anti-CD3 antibody, masked anti-CD3 antibody, activatable anti-HER2 antibody, or activatable anti-CD20 antibody) described herein comprises one or more antibody constant regions, such as human heavy chain constant regions and/or human light chain constant regions. In some embodiments, the human heavy chain constant region is of an isotype selected from IgA, IgG, and IgD. In some embodiments, the human light chain constant region is of an isotype selected from κ and λ. In some embodiments, the antibody comprises a human IgG constant region. In some embodiments, the antibody comprises a human IgG4 heavy chain constant region. In some embodiments, the antibody comprises a human IgG1 heavy chain constant region. In some such embodiments, the antibody comprises an S228P mutation in the human IgG4 constant region.

Whether or not effector function is desirable may depend on the particular method of treatment intended for an antibody. In some embodiments, when effector function is desirable, an antibody comprising a human IgG1 heavy chain constant region or a human IgG3 heavy chain constant region is selected. In some embodiments, when effector function is not desirable, an antibody comprising a human IgG4 or IgG2 heavy chain constant region is selected. In some embodiments, the antibody comprises a human IgG1 heavy chain constant region comprising one or more mutations that reduces effector function. In some embodiments, the antibody comprises an IgG1 heavy chain constant region comprising an N297A substitution.

The multispecific antibodies (including the activatable multispecific antibodies) described herein may comprise CH3 domains having one or more engineered disulfide bonds, one or more engineered (e.g., rearranged or inversed) salt bridges, or a combination thereof. Unless stated otherwise, all amino acid residue numbering herein is based on EU numbering, and the amino acid substitutions are relative to the wildtype (or naturally occurring) sequence at the corresponding amino acid positions in a wild type (or naturally occurring) CH3 domain sequence. It is appreciated that the mutations or substitutions described herein are applicable to all IgG subclasses and allotypes. IgG allotypes have been described, for example, in Jefferis R. and Lefranc M. mAbs 1:4, 1-7 (2009), which is incorporated herein by reference in its entirety. In some embodiments, the amino acid mutations or substitutions described herein are relative to a wildtype CH3 domain sequence of an IgG1, such as IgG1 allotype G1m, 1(a), 2(x), 3(f) or 17(z). In some embodiments, the amino acid mutations or substitutions described herein are relative to a wildtype CH3 domain sequence of an IgG4. For example, a D356K substitution relative to a wildtype CH3 domain of one human IgG1 allotype (Uniprot ID P01857; SEQ ID NO: 29) is equivalent to an E356K substitution relative to a wildtype CH3 domain of a second human IgG1 allotype (SEQ ID NO: 30), or a wildtype CH3 domain of a human IgG4 (SEQ ID NO: 31). Exemplary CH3 domain mutations are shown in Tables D and E. In some embodiments, the amino acid mutations or substitutions described herein are relative to a wildtype Fc region sequence, e.g., an IgG1 Fc region (SEQ ID NO: 32 or 33) or an IgG4 Fc region (SEQ ID NO: 34).

TABLE D
Fc mutations

| | Mutations (first CH3 | CH3 SEQ |
|---|---|---|
| Designs | domain-second CH3 domain) | ID NOs |
| Disulfide | N390C-S400′C | 24, 23 |
| bond | S400C-N390′C | 23, 24 |
| | K392C-V397′C | 25, 26 |
| | V397C-K392′C | 26, 25 |
| | K392C-S400′C | 27, 28 |
| | S400C-K392′C | 28, 27 |
| Charge | E357K:T411K-L351′D:K370′D | 9, 10 |
| designs | E357K:S364K-L351′D:K370′D | 11, 12 |
| | D356K:E357K:S364K-L351′D:K370′D:K439′D | 13, 14 |
| Charge + | E357K:S364K:N390C-L351′D:K370′D:S400′C | 15, 16 |
| disulfide | E357K:S364K:S400C-L351′D:K370′D:N390′C | 17, 18 |
| bond | D356K:E357K:S364K:N390C- | 19, 20 |
| designs | L351′D:K370′D:S400′C:K439′D | |
| | D356K:E357K:S364K:S400C- | 21, 22 |
| | L351′D:K370′D:N390′C:K439′D | |

TABLE E
Fc mutations ID.

| | CH3 | |
|---|---|---|
| Mutations | SEQ | |
| ID | ID NOs | Mutations (first CH3 domain-second CH3 domain) |
| TRF01 | — | T366S, L368A, Y407V, Y349C-T366′W, S354′C |
| TRF02 | — | T350V, L351Y, F405A, Y407V-T350′V, T366′L, K392′L, T394′W |
| TRF03 | — | K196Q, S228P, F296Y, E356K, R409K, H435R, L445P-K196′Q, |
| | | S228′P, F296′Y, R409′K, K439′E, L445′P |
| TYM01 | 1, 2 | T366S, L368A, Y407V, N390C-T366′W, S400′C |
| TYM02 | 3, 4 | T366S, L368A, Y407V, S400C-T366′W, N390′C |
| TYM03 | — | Y349C, L368V, Y407V-S354′C, T366′W |
| TYM04 | — | L368V, Y407V-T366′W |
| TYM05 | 5, 6 | L368V, Y407V, N390C-T366′W, S400′C |
| TYM06 | 7, 8 | L368V, Y407V, S400C-T366′W, N390′C |
| TYM07 | 9, 10 | E357K:T411K-L351′D:K370′D |
| TYM08 | 11, 12 | E357K:S364K-L351′D:K370′D |
| TYM09 | 13, 14 | D or E356K:E357K:S364K-L351′D:K370′D:K439′D |
| TYM10 | 15, 16 | E357K:S364K:N390C-L351′D:K370′D:S400′C |
| TYM11 | 17, 18 | E357K:S364K:S400C-L351′D:K370′D:N390′C |
| TYM12 | 19, 20 | D or E356K:E357K:S364K:N390C-L351′D:K370′D:S400′C:K439′D |
| TYM13 | 21, 22 | D or E356K:E357K:S364K:S400C-L351′D:K370′D:N390′C:K439′D |

In some embodiments, the multispecific antibody (e.g., the activatable multispecific antibody) comprises an engineered disulfide bond between C390 in a first CH3 domain and C400 in a second CH3 domain, between C392 in a first CH3 domain and C397 in a second CH3 domain, or between C392 in a first CH3 domain and C400 in a second CH3 domain. In some embodiments, the multispecific antibody (e.g., the activatable multispecific antibody) comprises a rearranged salt-bridge network as compared to wildtype CH3 domains, e.g., among positions 357 and 411 in a first CH3 domain and positions 351 and 370 in a second CH3 domain (e.g., E357K:T411K-L351′D:K370′D), or among positions 357 and 364 in a first CH3 domain and positions 351 and 370 in a second CH3 domain (e.g., E357K:S364K-L351′D:K370′D). In some embodiments, the multispecific antibody comprises an inversed salt bridge as compared to wildtype CH3 domains between position 356 in a first CH3 domain and position 439 in a second CH3 domain (e.g., D356-K439′). The multispecific antibodies having CH3 mutations may have high yield, superior stability (e.g., resistance to aggregation and precipitation at high temperature or due to freeze-thaw cycles), and potent activity.

In some embodiments, the multispecific antibody (e.g., the activatable multispecific antibody) comprises CH3 domains having any one or combination of engineered residues, which promote heterodimer formation as described herein. Heteromultimers comprising multiple heterodimers formed by a first polypeptide comprising a first engineered CH3 domain and a second polypeptide comprising a second engineered CH3 domain are also contemplated herein.

In some embodiments, the multispecific antibody (e.g., the activatable multispecific antibody) comprises: i) a first CH3 domain comprising a cysteine (C) residue at position 390 and a second CH3 domain comprising a cysteine residue at position 400, or a first CH3 domain comprising a cysteine residue at position 400 and a second CH3 domain comprising a cysteine residue at position 390; or ii) a first CH3 domain comprising a cysteine residue at position 392 and a second CH3 domain comprising a cysteine residue at position 397, or a first CH3 domain comprising a cysteine residue at position 397 and a second CH3 domain comprising a cysteine residue at position 392; or iii) a first CH3 domain comprising a cysteine residue at position 392 and a second CH3 domain comprising a cysteine residue at position 400, or a first CH3 domain comprising a cysteine residue at position 400 and a second CH3 domain comprising a cysteine residue at position 392; and wherein the amino acid residue numbering is based on EU numbering.

In some embodiments, the multispecific antibody (e.g., the activatable multispecific antibody) comprises: a first polypeptide comprising a first CH3 domain and a second polypeptide comprising a second CH3 domain, wherein: i) the first CH3 domain further comprises a positively charged residue at position 357 and the second CH3 domain further comprises a negatively charged residue at position 351, or the first CH3 domain further comprises a negatively charged residue at position 351 and the second CH3 domain further comprises a positively charged residue at position 357; or ii) the first CH3 domain further comprises a positively charged residue at position 411 and the second CH3 domain further comprises a negatively charged residue at position 370, or the first CH3 domain further comprises a negatively charged residue at position 370 and the second CH3 domain further comprises a positively charged residue at position 411; or iii) the first CH3 domain further comprises a positively charged residue at position 364 and the second CH3 domain further comprises a negatively charged residue at position 370, or the first CH3 domain further comprises a negatively charged residue at position 370 and the second CH3 domain further comprises a positively charged residue at position 364; or a combination of i) and ii), or a combination or i) and iii), wherein the amino acid residue numbering is based on EU numbering. In some embodiments, the first CH3 domain further comprises a positively charged residue at position 356 and the second CH3 domain further comprises a negatively charged residue at position 439, or first CH3 domain further comprises a negatively charged residue at position 439 and the second CH3 domain further comprises a positively charged residue at position 356, and wherein the amino acid residue numbering is based on EU numbering.

In some embodiments, the multispecific antibody (e.g., the activatable multispecific antibody) comprises: a first polypeptide comprising a first CH3 domain and a second polypeptide comprising a second CH3 domain, wherein: i) the first CH3 domain comprises a cysteine (C) residue at position 390 and the second CH3 domain comprises a cysteine residue at position 400, or the first CH3 domain comprises a cysteine residue at position 400 and the second CH3 domain comprises a cysteine residue at position 390; or ii) the first CH3 domain comprises a cysteine residue at position 392 and the second CH3 domain comprises a cysteine residue at position 397, or the first CH3 domain comprises a cysteine residue at position 397 and the second CH3 domain comprises a cysteine residue at position 392; or iii) the first CH3 domain comprises a cysteine residue at position 392 and the second CH3 domain comprises a cysteine residue at position 400, or the first CH3 domain comprises a cysteine residue at position 400 and the second CH3 domain comprises a cysteine residue at position 392; and wherein: a) the first CH3 domain further comprises a positively charged residue at position 357 and the second CH3 domain further comprises a negatively charged residue at position 351, or the first CH3 domain further comprises a negatively charged residue at position 351 and the second CH3 domain further comprises a positively charged residue at position 357; or b) the first CH3 domain further comprises a positively charged residue at position 411 and the second CH3 domain further comprises a negatively charged residue at position 370, or the first CH3 domain further comprises a negatively charged residue at position 370 and the second CH3 domain further comprises a positively charged residue at position 411; or c) the first CH3 domain further comprises a positively charged residue at position 364 and the second CH3 domain further comprises a negatively charged residue at position 370, or the first CH3 domain further comprises a negatively charged residue at position 370 and the second CH3 domain further comprises a positively charged residue at position 364; or a combination of a) and b), or a combination of a) and c); wherein the amino acid residue numbering is based on EU numbering. In some embodiments, the first CH3 domain further comprises a positively charged residue at position 356 and the second CH3 domain further comprises a negatively charged residue at position 439, or first CH3 domain further comprises a negatively charged residue at position 439 and the second CH3 domain further comprises a positively charged residue at position 356, and wherein the amino acid residue numbering is based on EU numbering.

The CH3 domains may be derived from any naturally occurring immunoglobulin molecules. In some embodiments, the CH3 domains are derived from an IgG1 molecule, an IgG2 molecule, an IgG3 molecule, or an IgG4 molecule. In some embodiments, the CH3 domains are human CH3 domains. In some embodiments, the CH3 domains are derived from human IgG1 molecules.

In some embodiments, the multispecific antibody (e.g., the activatable multispecific antibody) comprises: a first polypeptide comprising a first CH3 domain and a second polypeptide comprising a second CH3 domain, wherein: i) the first CH3 domain comprises N390C substitution and the second CH3 domain comprises S400C substitution, or the first CH3 domain comprises S400C substitution and the second CH3 domain comprises N390C substitution; or ii) the first CH3 domain comprises K392C substitution and the second CH3 domain comprises V397C substitution, or the first CH3 domain comprises V397C substitution and the second CH3 domain comprises K392C substitution; or iii) the first CH3 domain comprises K392C substitution and the second CH3 domain comprises S400C substitution, or the first CH3 domain comprises S400C substitution and the second CH3 domain comprises K392C substitution.

In some embodiments, the multispecific antibody (e.g., the activatable multispecific antibody) comprises: a first polypeptide comprising a first CH3 domain and a second polypeptide comprising a second CH3 domain, wherein: i) the first CH3 domain comprises E357K and T411K substitutions and the second CH3 domain comprises L351D and K370D substitutions, or the first CH3 domain comprises L351D and K370D substitutions and the second CH3 domain comprises E357K and T411K substitutions; or ii) the first CH3 domain comprises E357K and S364K substitutions and the second CH3 domain comprises L351D and K370D substitutions, or the first CH3 domain comprises L351D and K370D substitutions and the second CH3 domain comprises E357K and S364K substitutions; or iii) the first CH3 domain comprises D356K, E357K and S364K substitutions and the second CH3 domain comprises L351D, K370D and K439D substitutions, or the first CH3 domain comprises L351D, K370D and K439D substitutions and the second CH3 domain comprises D356K, E357K and S364K substitutions.

In some embodiments, the multispecific antibody (e.g., the activatable multispecific antibody) comprises: a first polypeptide comprising a first CH3 domain and a second polypeptide comprising a second CH3 domain, wherein the first CH3 domain comprises E357K, S364K and N390C substitutions and the second CH3 domain comprises L351D, K370D, and S400C substitutions, or the first CH3 domain comprises L351D, K370D, and S400C substitutions and the second CH3 domain comprises E357K, S364K and N390C substitutions.

In some embodiments, the multispecific antibody (e.g., the activatable multispecific antibody) comprises: a first polypeptide comprising a first CH3 domain and a second polypeptide comprising a second CH3 domain, wherein the first CH3 domain comprises E357K, S364K and S400C substitutions and the second CH3 domain comprises L351D, K370D, and N390C substitutions, or the first CH3 domain comprises L351D, K370D, and N390C substitutions and the second CH3 domain comprises E357K, S364K and S400C substitutions.

In some embodiments, the multispecific antibody (e.g., the activatable multispecific antibody) comprises: a first polypeptide comprising a first CH3 domain and a second polypeptide comprising a second CH3 domain, wherein the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions.

In some embodiments, the multispecific antibody (e.g., the activatable multispecific antibody) comprises: a first polypeptide comprising a first CH3 domain and a second polypeptide comprising a second CH3 domain, wherein the first CH3 domain comprises D356K, E357K, S364K and N390C substitutions and the second CH3 domain comprises L351D, K370D, K439D and S400C substitutions, or the first CH3 domain comprises L351D, K370D, K439D and S400C substitutions and the second CH3 domain comprises D356K, E357K, S364K and N390C substitutions.

In some embodiments, the multispecific antibody (e.g., the activatable multispecific antibody) comprises an IgG Fc region that comprises the engineered CH3 domains. The Fc region may be derived from any suitable Fc subclasses, including, but not limited to, IgG1, IgG2, IgG3, and IgG4 subclasses.

Exemplary polypeptide sequences of CH3 domains (or Fc regions) comprising the engineered disulfide bond(s) and/or salt bridge(s) described herein include SEQ ID NOs: 1-28.

In some embodiments, the multispecific antibody (e.g., the activatable multispecific antibody) comprises: a first polypeptide comprising the amino acid sequence of SEQ ID NO: 1, and a second polypeptide comprising the amino acid sequence of SEQ ID NO: 2. In some embodiments, the multispecific antibody comprises: a first polypeptide comprising the amino acid sequence of SEQ ID NO: 3, and a second polypeptide comprising the amino acid sequence of SEQ ID NO: 4. In some embodiments, the multispecific antibody comprises: a first polypeptide comprising the amino acid sequence of SEQ ID NO: 5, and a second polypeptide comprising the amino acid sequence of SEQ ID NO: 6. In some embodiments, the multispecific antibody comprises: a first polypeptide comprising the amino acid sequence of SEQ ID NO: 7, and a second polypeptide comprising the amino acid sequence of SEQ ID NO: 8. In some embodiments, the multispecific antibody comprises: a first polypeptide comprising the amino acid sequence of SEQ ID NO: 9, and a second polypeptide comprising the amino acid sequence of SEQ ID NO: 10. In some embodiments, the multispecific antibody comprises: a first polypeptide comprising the amino acid sequence of SEQ ID NO: 11, and a second polypeptide comprising the amino acid sequence of SEQ ID NO: 12. In some embodiments, the multispecific antibody comprises: a first polypeptide comprising the amino acid sequence of SEQ ID NO: 13, and a second polypeptide comprising the amino acid sequence of SEQ ID NO: 14. In some embodiments, the multispecific antibody comprises: a first polypeptide comprising the amino acid sequence of SEQ ID NO: 15, and a second polypeptide comprising the amino acid sequence of SEQ ID NO: 16. In some embodiments, the multispecific antibody comprises: a first polypeptide comprising the amino acid sequence of SEQ ID NO: 17, and a second polypeptide comprising the amino acid sequence of SEQ ID NO: 18. In some embodiments, the multispecific antibody comprises: a first polypeptide comprising the amino acid sequence of SEQ ID NO: 19, and a second polypeptide comprising the amino acid sequence of SEQ ID NO: 20. In some embodiments, the multispecific antibody comprises: a first polypeptide comprising the amino acid sequence of SEQ ID NO: 21, and a second polypeptide comprising the amino acid sequence of SEQ ID NO: 22. In some embodiments, the multispecific antibody comprises: a first polypeptide comprising the amino acid sequence of SEQ ID NO: 23, and a second polypeptide comprising the amino acid sequence of SEQ ID NO: 24.

### Cysteine Mutations

In some embodiments, the multispecific antibodies described herein (e.g., the activatable multispecific antibodies described herein) comprise a first polypeptide comprising a first CH3 domain and a second polypeptide comprising a second CH3 domain, wherein the first CH3 domain comprises a first engineered cysteine residue and the second CH3 domain comprises a second engineered cysteine residue, wherein the first engineered cysteine residue and the second cysteine residue form a disulfide bond.

In some embodiments, the first CH3 domain comprises a C at position 390 and the second CH3 domain comprises a C at position 400, or the first CH3 domain comprises a C at position 400 and the second CH3 domain comprises a C at position 390. In some embodiments, the first CH3 domain comprises N390C substitution and the second CH3 domain comprises S400C substitution, or the first CH3 domain comprises S400C substitution and the second CH3 domain comprises N390C substitution.

In some embodiments, the first CH3 domain comprises a C at position 392 and the second CH3 domain comprises a C at position 397, or the first CH3 domain comprises a C at position 397 and the second CH3 domain comprises a C at position 392. In some embodiments, the first CH3 domain comprises K392C substitution and the second CH3 domain comprises V397C substitution, or the first CH3 domain comprises V397C substitution and the second CH3 domain comprises K392C substitution.

In some embodiments, the first CH3 domain comprises a C at position 392 and the second CH3 domain comprises a C at position 400, or the first CH3 domain comprises a C at position 400 and the second CH3 domain comprises a C at position 392. In some embodiments, the first CH3 domain comprises K392C substitution and the second CH3 domain comprises S400C substitution, or the first CH3 domain comprises S400C substitution and the second CH3 domain comprises K392C substitution.

### Salt Bridge Mutations

In some embodiments, the multispecific antibodies described herein (e.g., the activatable multispecific antibodies described herein) comprise a first polypeptide comprising a first CH3 domain and a second polypeptide comprising a second CH3 domain, wherein the first CH3 domain comprises an engineered positively charged residue and the second CH3 domain comprises an engineered negatively charged residue, wherein the engineered positively charged residue and the engineered negatively charged residue form a salt bridge. The engineered salt bridge may introduce new salt bridges between the CH3 domains, rearrange a salt-bridge network among two or more amino acid residues, or reverse the charges on the amino acid residues forming the salt bridge (i.e., “inverse” a salt bridge) with respect to wildtype CH3 domains. In some embodiments, the engineered positively charged residue substitutes a negatively charged residue in a wildtype CH3 domain with a positively charged residue. In some embodiments, the engineered negatively charged residue substitutes a positively charged residue in a wildtype CH3 domain with a negatively charged residue. The rearranged and inversed salt bridges may result in changes in the isoelectric points (PI) of the heterodimer and the homodimer comprising the engineered CH3 domains, thereby allowing better separation of the heterodimer from the homodimer in a purification process.

In some embodiments, the first CH3 domain comprises a positively charged residue at position 357 and the second CH3 domain comprises a negatively charged residue at position 351, or the first CH3 domain comprises a negatively charged residue at position 351 and the second CH3 domain comprises a positively charged residue at position 357. In some embodiments, first CH3 domain comprises a K at position 357 and the second CH3 domain comprises a D at position 351, or the first CH3 domain comprises a D at position 351 and the second CH3 domain comprises a K at position 357. In some embodiments, first CH3 domain comprises a K at position 357 and the second CH3 domain comprises an E at position 351, or the first CH3 domain comprises an E at position 351 and the second CH3 domain comprises a K at position 357. In some embodiments, first CH3 domain comprises an R at position 357 and the second CH3 domain comprises a D at position 351, or the first CH3 domain comprises a D at position 351 and the second CH3 domain comprises an R at position 357. In some embodiments, first CH3 domain comprises an R at position 357 and the second CH3 domain comprises an E at position 351, or the first CH3 domain comprises an E at position 351 and the second CH3 domain comprises an R at position 357. In some embodiments, the first CH3 domain comprises E357K substitution and the second CH3 domain comprises L351D substitution, or the first CH3 domain comprises L351D substitution and the second CH3 domain comprises E357K substitution.

In some embodiments, the first CH3 domain comprises a positively charged residue at position 411 and the second CH3 domain comprises a negatively charged residue at position 370, or the first CH3 domain comprises a negatively charged residue at position 370 and the second CH3 domain comprises a positively charged residue at position 411. In some embodiments, first CH3 domain comprises a K at position 411 and the second CH3 domain comprises a D at position 370, or the first CH3 domain comprises a D at position 370 and the second CH3 domain comprises a K at position 411. In some embodiments, first CH3 domain comprises a K at position 411 and the second CH3 domain comprises an E at position 370, or the first CH3 domain comprises an E at position 370 and the second CH3 domain comprises a K at position 411. In some embodiments, first CH3 domain comprises an R at position 411 and the second CH3 domain comprises a D at position 370, or the first CH3 domain comprises a D at position 370 and the second CH3 domain comprises an R at position 411. In some embodiments, first CH3 domain comprises an R at position 411 and the second CH3 domain comprises an E at position 370, or the first CH3 domain comprises an E at position 370 and the second CH3 domain comprises an R at position 411. In some embodiments, the first CH3 domain comprises T411K substitution and the second CH3 domain comprises K370D substitution, or the first CH3 domain comprises K370D substitution and the second CH3 domain comprises T411K substitution.

In some embodiments, the first CH3 domain comprises a positively charged residue at position 364 and the second CH3 domain comprises a negatively charged residue at position 370, or the first CH3 domain comprises a negatively charged residue at position 370 and the second CH3 domain comprises a positively charged residue at position 364. In some embodiments, first CH3 domain comprises a K at position 364 and the second CH3 domain comprises a D at position 370, or the first CH3 domain comprises a D at position 370 and the second CH3 domain comprises a K at position 364. In some embodiments, first CH3 domain comprises a K at position 364 and the second CH3 domain comprises an E at position 370, or the first CH3 domain comprises an E at position 370 and the second CH3 domain comprises a K at position 364. In some embodiments, first CH3 domain comprises an R at position 364 and the second CH3 domain comprises a D at position 370, or the first CH3 domain comprises a D at position 370 and the second CH3 domain comprises an R at position 364. In some embodiments, first CH3 domain comprises an R at position 364 and the second CH3 domain comprises an E at position 370, or the first CH3 domain comprises an E at position 370 and the second CH3 domain comprises an R at position 364. In some embodiments, the first CH3 domain comprises S364K substitution and the second CH3 domain comprises K370D substitution, or the first CH3 domain comprises K370D substitution and the second CH3 domain comprises S364K substitution.

In some embodiments, the first CH3 domain comprises a positively charged residue at position 356 and the second CH3 domain comprises a negatively charged residue at position 439, or the first CH3 domain comprises a negatively charged residue at position 439 and the second CH3 domain comprises a positively charged residue at position 356. In some embodiments, first CH3 domain comprises a K at position 356 and the second CH3 domain comprises a D at position 439, or the first CH3 domain comprises a D at position 439 and the second CH3 domain comprises a K at position 356. In some embodiments, first CH3 domain comprises a K at position 356 and the second CH3 domain comprises an E at position 439, or the first CH3 domain comprises an E at position 439 and the second CH3 domain comprises a K at position 356. In some embodiments, first CH3 domain comprises an R at position 356 and the second CH3 domain comprises a D at position 439, or the first CH3 domain comprises a D at position 439 and the second CH3 domain comprises an R at position 356. In some embodiments, first CH3 domain comprises an R at position 356 and the second CH3 domain comprises an E at position 439, or the first CH3 domain comprises an E at position 439 and the second CH3 domain comprises an R at position 356. In some embodiments, the first CH3 domain comprises D356K substitution and the second CH3 domain comprises K439D substitution, or the first CH3 domain comprises K439D substitution and the second CH3 domain comprises D356K substitution.

Any of the engineered salt bridges described herein may be combined with each other. In some embodiments, the first CH3 domain comprises a positively charged residue at position 357 and a positively charged residue at position 411 and the second CH3 domain comprises a negatively charged residue at position 351 and a negatively charged residue at position 370, or the first CH3 domain comprises a negatively charged residue at position 351 and a negatively charged residue at position 370 and the second CH3 domain comprises a positively charged residue at position 357 and a positively charged residue at position 411. In some embodiments, the first CH3 domain comprises E357K and T411K substitutions and the second CH3 domain comprises L351D and K370D substitutions, or the first CH3 domain comprises L351D and K370D substitutions and the second CH3 domain comprises E357K and T411K substitutions.

In some embodiments, the first CH3 domain comprises a positively charged residue at position 357 and a positively charged residue at position 364 and the second CH3 domain comprises a negatively charged residue at position 351 and a negatively charged residue at position 370, or the first CH3 domain comprises a negatively charged residue at position 351 and a negatively charged residue at position 370 and the second CH3 domain comprises a positively charged residue at position 357 and a positively charged residue at position 364. In some embodiments, the first CH3 domain comprises E357K and S364K substitutions and the second CH3 domain comprises L351D and K370D substitutions, or the first CH3 domain comprises L351D and K370D substitutions and the second CH3 domain comprises E357K and S364K substitutions.

In some embodiments, the first CH3 domain comprises a positively charged residue at position 356, a positively charged residue at position 357, and a positively charged residue at position 364 and the second CH3 domain comprises a negatively charged residue at position 351, a negatively charged residue at position 370, and a negatively charged residue at position 439, or the first CH3 domain comprises a negatively charged residue at position 351, a negatively charged residue at position 370, and a negatively charged residue at position 439 and the second CH3 domain comprises a positively charged residue at position 356, a positively charged residue at position 357, and a positively charged residue at position 364. In some embodiments, the first CH3 domain comprises D356K, E357K and S364K substitutions and the second CH3 domain comprises L351D, K370D and K439D substitutions, or the first CH3 domain comprises L351D, K370D and K439D substitutions and the second CH3 domain comprises D356K, E357K and S364K substitutions.

### Other Mutations

The CH3 domains or the Fc regions described herein may further comprise engineered disulfide bonds and/or salt bridges listed in Table F below.

TABLE F
Exemplary Fc mutations.

| Mutation(s) in first polypeptide chain | Mutation(s) in second polypeptide chain |
|---|---|
| F405L | K409R |
| S364H, F405A | Y349T, T394F |
| S364K, E357Q | L368D, K370S |
| T366W | T366S, L368A, Y407V |
| S354C, T366W | Y349C, T366S, L368A, Y407V |
| T350, L351, F405, Y407 | T350, T366, K392, T394 |
| T350 is T350V, T350I, T350L or T350M | T350 is T350V, T350I, T350L or T350M |
| L351 is L351Y | T366 is T366L, T366I, T366V or T366M |
| F450 is F450A, F450V, F450T or F450S | K392 is K392F, K392L or K392M |
| Y407 is Y407V, Y407A or Y407I | T394 is T394W |
| D399K, E356K | K409D, K392D |
| D221E, P228E, L368E | D221R, P228R, K409R |
| C223E, E225E, P228E, L368E | C223R, E225R, P228R, K409R |
| H435R | None |
| K196Q, S228P, F296Y, E356K, R409K, | K196Q, S228P, F296Y, R409K, K439E, L445P |
| H435R, L445P | |
| K409W | D399V/F405T |
| K360E | Q347R |
| Y349C/K360E/K409W | Q347R/S354C/D399V/F405T |
| K360E/K409W | Q347R/D399V/F405T |
| Y349S/K409W | E357W/D399V/F405T |
| Y349S/S354C/K409W | Y349C/E357W/D399V/F405T |
| T366K | L351D |
| Y349E or D and L368E | L351D |
| Y349C/T366W | D356C/T366S/L368A/Y407V/F405K |
| Y349C/T366W/F405K | D356C/T366S/L368A/Y407V |
| Y349C/T366W/K409E | D356C/T366S/L368A/Y407V/F405K |
| Y349C/T366W/K409A | D356C/T366S/L368A/Y407V/F405K |
| S364K | L368D |
| S364K | K370S |
| F405K | K409F |
| F405R | K409F |
| S364K/K409F | L368D/F405R |
| S364K/K409F | K370S/F405R |
| S364K/K409W | K370S/F405R |
| K370E or E356K and K409R | E357K and K409R or K370E |
| 354/364/407 | 347/349/350/351/366/368/370/407 |
| 349 | 351/354/357/360/364/366/368/407 |
| P395K/P396K/V397K | T394D/P395D/P396D |
| F405E/Y407E/K409E | F405K/Y407K |
| M428S/N434S/Y436H | H435R |

In some embodiments, the first CH3 domain further comprises a C at position 392 and the second CH3 domain comprises a C at position 399, or the first CH3 domain comprises a C at position 399 and the second CH3 domain comprises a C at position 392. In some embodiments, the first CH3 domain further comprises K392C substitution and the second CH3 domain further comprises D399C substitution, or the first CH3 domain further comprises D399C substitution and the second CH3 domain further comprises K392C substitution.

In some embodiments, the first CH3 domain further comprises a C at position 394 and the second CH3 domain comprises a C at position 354, or the first CH3 domain comprises a C at position 354 and the second CH3 domain comprises a C at position 394. In some embodiments, the first CH3 domain further comprises Y394C substitution and the second CH3 domain further comprises S354C substitution, or the first CH3 domain further comprises S354C substitution and the second CH3 domain further comprises Y394C substitution.

In some embodiments, the first CH3 domain further comprises a C at position 356 and the second CH3 domain comprises a C at position 349, or the first CH3 domain comprises a C at position 349 and the second CH3 domain comprises a C at position 356. In some embodiments, the first CH3 domain further comprises D356C substitution and the second CH3 domain further comprises Y349C substitution, or the first CH3 domain further comprises Y349C substitution and the second CH3 domain further comprises D356C substitution.

In some embodiments, the first CH3 domain further comprises K392D and K409D substitutions and the second CH3 domain further comprises D356K and D399K substitutions, or the first CH3 domain further comprises D356K and D399K substitutions and the second CH3 domain further comprises K392D and K409D substitutions.

In some embodiments, the first CH3 domain further comprises L368D and K370S substitutions and the second CH3 domain further comprises E357Q and S364K substitutions, or the first CH3 domain further comprises E357Q and S364K substitutions and the second CH3 domain further comprises L368D and K370S substitutions.

In some embodiments, the first CH3 domain further comprises L351K and T366K substitutions and the second CH3 domain further comprises L351D and L368E substitutions, or the first CH3 domain further comprises L351D and L368E substitutions and the second CH3 domain further comprises L351K and T366K substitutions.

In some embodiments, the first CH3 domain further comprises P395K, P396K and V397K substitutions and the second CH3 domain comprises T394D, P395D and P396D substitutions, or the first CH3 domain further comprises T394D, P395D and P396D substitutions and the second CH3 domain further comprises P395K, P396K and V397K substitutions.

In some embodiments, the first CH3 domain further comprises F405E, Y407E and K409E substitutions and the second CH3 domain comprises F405K and Y407K substitutions, or the first CH3 domain further comprises F405K and Y407K substitutions and the second CH3 domain further comprises F405E, Y407E and K409E substitutions.

The multispecific antibodies (e.g., the activatable multispecific antibodies) comprising engineered CH3 domains disulfide bonds and/or salt bridges described herein may further comprise one or more knob-into-hole residues. “Knob-into-hole” or “KIH” refers to an approach known in the art for making bispecific antibodies also known as the “protuberance-into-cavity” approach (see, e.g., U.S. Pat. No. 5,731,168). In this approach, two immunoglobulin polypeptides (e.g., heavy chain polypeptides) each comprise an interface. An interface of one immunoglobulin polypeptide interacts with a corresponding interface on the other immunoglobulin polypeptide, thereby allowing the two immunoglobulin polypeptides to associate. These interfaces may be engineered such that a “knob” or “protuberance” (these terms may be used interchangeably herein) located in the interface of one immunoglobulin polypeptide corresponds with a “hole” or “cavity” (these terms may be used interchangeably herein) located in the interface of the other immunoglobulin polypeptide. In some embodiments, the hole is of identical or similar size to the knob and suitably positioned such that when the two interfaces interact, the knob of one interface is positionable in the corresponding hole of the other interface. Without wishing to be bound to theory, this is thought to stabilize the heteromultimer and favor formation of the heteromultimer over other species, for example homomultimers. In some embodiments, the KIH approach is used in combination with the engineered disulfide bonds and/or salt bridges described herein to promote the heteromultimerization of two different immunoglobulin polypeptides, creating a bispecific antibody comprising two immunoglobulin polypeptides with binding specificities for different epitopes. In some embodiments, the CH3 domains of the activatable multispecific antibody described herein do not comprise KIH residues.

In some embodiments, the first CH3 domain further comprises T336S, L368A and Y407V substitutions and the second CH3 domain further comprises T366W substitution, or the first CH3 domain further comprises T366W substitution and the second CH3 domain further comprises T336S, L368A and Y407V substitutions.

In some embodiments, the first CH3 domain comprises L368V and Y407V substitutions and the second CH3 domain comprises T366W substitution, or the first CH3 domain comprises T366W substitution and the second CH3 domain comprises L368V and Y407V substitutions.

### K. Multispecific Antibodies

Also provided are multispecific antibodies that correspond to the activatable multispecific antibodies or masked multispecific antibodies described herein. In some embodiments, the multispecific antibody is a bispecific antibody or a trispecific antibody. In some embodiments, the multispecific antibody is a BiTE molecule. In some embodiments, the multispecific antibody is a HER2xCD3 bispecific antibody that specifically binds to HER2 and CD3. In some embodiments, the multispecific antibody is a CD20xCD3 bispecific antibody that specifically binds to CD20 and CD3. In some embodiments, the multispecific antibody specifically binds CD3 with a weak affinity, e.g., an EC₅₀ of at least 10 nM (e.g., at least 100 nM) as determined by an ELISA assay (e.g., as described in Example 5), and/or a Kd of at least 50 nM. In some embodiments, the multispecific antibody does not comprise any masking moiety or cleavable moiety. In some embodiments, the multispecific antibody is obtained upon cleavage of the cleavable moiety or cleavable moieties.

In some embodiments, there is provided a multispecific antibody comprising: a) a first antigen-binding fragment comprising a VH1 and a VL1 of an anti-CD3 antibody that specifically binds CD3; and b) a second antigen-binding fragment comprising a VH2 and a VL2 of an antibody that specifically binds a target antigen (e.g., a tumor antigen, such as HER2, CD20, TROP2, BCMA, or CD19). In some embodiments, the first antigen-binding fragment is selected from the group consisting of a Fab, a Fv, a scFab and a scFv. In some embodiments, the second antigen-binding fragment is selected from the group consisting of a Fab, a Fv, a scFab and a scFv. In some embodiments, the first antigen-binding fragment is a Fab and the second antigen-binding fragment is a Fab. In some embodiments, the first antigen-binding fragment is a Fab and the second antigen-binding fragment is a scFv.

In some embodiments, there is provided a bispecific T cell engager (BiTE) molecule targeting CD3 and a tumor antigen (e.g., HER2, CD20, TROP2, BCMA, or CD19), comprising a first polypeptide, a second polypeptide and a third polypeptide, wherein:
(i) the first polypeptide comprises a structure represented by the formula:

VH1-CH1-hinge-CH2-first CH3 (5a);

(ii) the second polypeptide comprises a structure represented by the formula:

scFv-hinge-CH2-second CH3 (5b);

and
(iii) the third polypeptide comprises a structure represented by the formula:

VL1-CL (5c);

wherein:
VL1 is a first immunoglobulin light chain variable domain;
VH2 is a first immunoglobulin heavy chain variable domain;
scFv is a single-chain variable fragment comprising a second immunoglobulin light chain
variable domain (VL2) and a second immunoglobulin heavy chain variable domain (VH2);
CL is an immunoglobulin light chain constant domain;
CH1 is an immunoglobulin heavy chain constant domain 1;
CH2 is an immunoglobulin heavy chain constant domain 2; and
hinge is an immunoglobulin hinge region connecting the CH1 and CH2 domains; wherein VL and VH associate to form a first Fv that specifically binds the tumor antigen (e.g., HER2, CD20, TROP2, BCMA, or CD19); and wherein the scFv specifically binds CD3. In some embodiments, the scFv binds CD3 with half-maximal binding at a concentration of antibody (EC₅₀) that is at least 10 nM (e.g., at least 100 nM) as determined by an ELISA assay (e.g., as described in Example 5). In some embodiments, the scFv binds CD3 with a dissociation constant (Kd) of at least 50 nM.

In some embodiments, there is provided a bispecific T cell engager (BiTE) molecule targeting CD3 and a tumor antigen (e.g., HER2, CD20, TROP2, BCMA, or CD19), comprising a first polypeptide, a second polypeptide, a third polypeptide and a fourth polypeptide, wherein:
(i) the first polypeptide comprises a structure represented by the formula:

VH1-CH1-hinge-CH2-first CH3 (6a);

(ii) the second polypeptide comprises a structure represented by the formula:

VH2-CH1-hinge-CH2-second CH3 (6b);

(iii) the third polypeptide comprises a structure represented by the formula:

VL1-CL (6c);

and
(iv) the fourth polypeptide comprises a structure represented by the formula:

VL2-CL (6d);

wherein:
VL1 is a first immunoglobulin light chain variable domain;
VH1 is a first immunoglobulin heavy chain variable domain;
VL2 is a second immunoglobulin light chain variable domain;
VH2 is a second immunoglobulin heavy chain variable domain;
CL is an immunoglobulin light chain constant domain;
CH1 is an immunoglobulin heavy chain constant domain 1;
CH2 is an immunoglobulin heavy chain constant domain 2; and
hinge is an immunoglobulin hinge region connecting the CH1 and CH2 domains; wherein VL1 and VH1 associate to form a first Fv that specifically binds the tumor antigen (e.g., HER2, CD20, TROP2, BCMA, or CD19); and wherein VL2 and VH2 associate to form a second Fv that specifically binds CD3. In some embodiments, the second Fv binds CD3 with half-maximal binding at a concentration of antibody (EC₅₀) that is at least 10 nM (e.g., at least 100 nM) as determined by an ELISA assay (e.g., as described in Example 5). In some embodiments, the second Fv binds CD3 with a dissociation constant (K_{d}) of at least 50 nM.

In some embodiments, the scFv or the second Fv comprises a VH2 comprising a CDR-H1 comprising the amino acid sequence of SEQ ID NO: 61, a CDR-H2 comprising the amino acid sequence of SEQ ID NO: 62, and a CDR-H3 comprising the amino acid sequence of SEQ ID NO: 63; and/or a VL2 comprising a CDR-L1 comprising the amino acid sequence of SEQ ID NO: 64, a CDR-L2 comprising the amino acid sequence of SEQ ID NO: 65, and a CDR-L3 comprising the amino acid sequence of SEQ ID NO: 66. In some embodiments, the scFv or the second Fv comprises a VH2 comprising the amino acid sequence of SEQ ID NO: 67, and/or a VL2 comprising the amino acid sequence of SEQ ID NO: 68. In some embodiments, the scFv comprises the amino acid sequence of SEQ ID NO: 79.

In some embodiments, the scFv or the second Fv comprises a VH2 comprising a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and/or a VL2 comprising a CDR-L1 comprising the amino acid sequence of SEQ ID NO: 397, a CDR-L2 comprising the amino acid sequence of SEQ ID NO: 398, and a CDR-L3 comprising the amino acid sequence of SEQ ID NO: 400. In some embodiments, the scFv or the second Fv comprises a VH2 comprising the amino acid sequence of SEQ ID NO: 402, and/or a VL2 comprising the amino acid sequence of SEQ ID NO: 403. In some embodiments, the scFv comprises the amino acid sequence of SEQ ID NO: 421.

In some embodiments, the scFv or the second Fv comprises a VH2 comprising a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and/or a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the scFv or the second Fv comprises a VH2 comprising the amino acid sequence of SEQ ID NO: 410, and/or a VL2 comprising the amino acid sequence of SEQ ID NO: 411. In some embodiments, the scFv comprises the amino acid sequence of SEQ ID NO: 422.

In some embodiments, the first Fv specifically binds HER2. In some embodiments, the first Fv comprises a VH1 comprising an CDR-H1 comprising the amino acid sequence of SEQ ID NO: 69, a CDR-H2 comprising the amino acid sequence of SEQ ID NO: 70, and a CDR-H3 comprising the amino acid sequence of SEQ ID NO: 71; and/or a VL1 comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the first Fv comprises a VH1 comprising an CDR-H1 comprising the amino acid sequence of SEQ ID NO: 423, a CDR-H2 comprising the amino acid sequence of SEQ ID NO: 424, and a CDR-H3 comprising the amino acid sequence of SEQ ID NO: 71; and/or a VL1 comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74. In some embodiments, the first Fv comprises a VH1 comprising the amino acid sequence of SEQ ID NO: 75 and/or a VL1 comprising the amino acid sequence of SEQ ID NO: 76.

In some embodiments, the first Fv specifically binds CD20. In some embodiments, the first Fv comprises a VH1 comprising an CDR-H1 comprising the amino acid sequence of SEQ ID NO: 556, a CDR-H2 comprising the amino acid sequence of SEQ ID NO: 557, and a CDR-H3 comprising the amino acid sequence of SEQ ID NO: 558; and/or a VL1 comprising a CDR-L1 comprising the amino acid sequence of SEQ ID NO: 559, a CDR-L2 comprising the amino acid sequence of SEQ ID NO: 560, and a CDR-L3 comprising the amino acid sequence of SEQ ID NO: 561. In some embodiments, the first Fv comprises a VH1 comprising the amino acid sequence of SEQ ID NO: 562 and/or a VL1 comprising the amino acid sequence of SEQ ID NO: 563.

In some embodiments, the first CH3 domain comprises D356K, E357K, S364K and S400C substitutions and the second CH3 domain comprises L351D, K370D, N390C and K439D substitutions, or the first CH3 domain comprises L351D, K370D, N390C and K439D substitutions and the second CH3 domain comprises D356K, E357K, S364K and S400C substitutions. In some embodiments, the bispecific T cell engager (BiTE) molecule comprises an IgG1 Fc region, such as an IgG1 Fc having an N297A substitution.

In some embodiments, there is provided a bispecific T cell engager molecule comprising a first polypeptide comprising the amino acid sequence of SEQ ID NO: 112, a second polypeptide comprising the amino acid sequence of SEQ ID NO: 113, and a third polypeptide comprising the amino acid sequence of SEQ ID NO: 114.

In some embodiments, there is provided a bispecific T cell engager molecule comprising a first polypeptide comprising the amino acid sequence of SEQ ID NO: 564, a second polypeptide comprising the amino acid sequence of SEQ ID NO: 565, and a third polypeptide comprising the amino acid sequence of SEQ ID NO: 566.

In some embodiments, there is provided a bispecific T cell engager molecule comprising a first polypeptide comprising the amino acid sequence of SEQ ID NO: 564, a second polypeptide comprising the amino acid sequence of SEQ ID NO: 565, and a third polypeptide comprising the amino acid sequence of SEQ ID NO: 568.

In some embodiments, the multispecific antibody (e.g., the CD20xCD3 BiTE, HER2xCD3 BiTE, TROP2xCD3 BiTE, BCMAxCD3 BiTE, or CD19xCD3 BiTE) is more highly expressed than a reference antibody (e.g., an anti-CD20, anti-CD3, anti-HER2, anti-TROP2, anti-BCMA, or anti-CD19 antibody that is not in a multispecific format). In some embodiments, the multispecific antibody (e.g., the CD20xCD3 BiTE, HER2xCD3 BiTE, TROP2xCD3 BiTE, BCMAxCD3 BiTE, or CD19xCD3 BiTE) is produced to a higher abundance of protein than a reference antibody (e.g., an anti-CD20, antiCD3, anti-HER2, anti-TROP2, anti-BCMA, or anti-CD19 antibody that is not in a multispecific format). In some embodiments, the multispecific antibody (e.g., the CD20xCD3 BiTE, HER2xCD3 BiTE, TROP2xCD3 BiTE, BCMAxCD3 BiTE, or CD19xCD3 BiTE) is more likely to be properly folded than a reference antibody (e.g., an anti-CD20, antiCD3, anti-HER2, anti-TROP2, anti-BCMA, or anti-CD19 antibody that is not in a multispecific format). In some embodiments, the expression, protein abundance, or level of proper folding compared to a reference antibody is measured under controlled experimental conditions. In some embodiments, the multispecific antibody is an activatable multispecific antibody. In some embodiments, the multispecific antibody is an unmasked multispecific antibody.

### L. Masked Antibodies

Also provided are masked antibodies. In some embodiments, a masked antibody comprises any one of the target binding moieties described in Section H. Target binding moiety (TBM), and any one of the masking moieties described in Section F. Masking Moiety (MM). In some embodiments, a masked antibody further comprises a cleavable moiety. In some embodiments, the masked antibody is an activatable antibody. See, Section A. Activatable multispecific T cell engagers, Section C. Activatable anti-CD3 antibodies and Section D. Activatable anti-HER2 antibodies. In some embodiments, a masked antibody further comprises a non-cleavable linker.

### i) Masked Anti-CD3 Antibodies

Also provided herein are masked antibodies (“masked anti-CD3 antibodies”) that target CD3 (e.g., human CD3). The masked antibodies may be derived from any anti-CD3 antibodies known in the art, including, but not limited to, SP34, OKT3, as well as variants, mutants and derivatives thereof.

The present application provides masked antibodies, masked antibody fragments, and polypeptides that target CD3, comprising a masking moiety (MM) comprising an amino acid sequence selected from the group consisting of SEQ ID NOs: 35, 417, 585-588, and 597-599.

The present application also provides masked antibodies, masked antibody fragments, and polypeptides that target CD3, comprising a masking moiety (MM) comprising the amino acid sequence of EVGSY (SEQ ID NO: 667) at the N-terminus of the MM. Further, the present application provides masked antibodies, masked antibody fragments, and polypeptides that target CD3, comprising a masking moiety (MM) comprising an amino acid sequence according to Formula (IX): PYDDPDCPSHX₁SDCDX₂ (SEQ ID NO: 668), wherein X₁ is D or E, and X₂ is N or Q. The present application also provides masked antibodies, masked antibody fragments, and polypeptides that target CD3, comprising a masking moiety (MM) comprising an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y.

a) Masked Anti-CD3 Antibodies Derived from SP34 and Low-Affinity Mutants

The present application provides masked antibodies, masked antibody fragments, and polypeptides that target CD3, comprising a masking moiety (MM). In some embodiments, the masked antibody comprises a MM comprising the amino acid sequence of EVGSY (SEQ ID NO: 667) at the N-terminus of the MM. In some embodiments, the masked antibody comprises a MM comprising an amino acid sequence according to Formula (IX): PYDDPDCPSHX₁SDCDX₂ (SEQ ID NO: 668), wherein X₁ is D or E, and X₂ is N or Q. In some embodiments, the MM comprise an amino acid sequence selected from the group consisting of SEQ ID NOs: 35, 417 and 597-599. In some embodiments, the MM comprises the amino acid sequence of SEQ ID NO: 35. In some embodiments, the MM comprises the amino acid sequence of SEQ ID NO: 35 or 417.

In some embodiments, there is provided an antibody light chain comprising a polypeptide comprising, from N-terminus to C-terminus, a MM, a non-cleavable linked (NCL), and a target binding moiety (TBM), wherein the MM comprises an amino acid sequence selected from the group consisting of SEQ ID NOs: 35, 417, and 597-599; and wherein the TBM comprises a VL of an anti-CD3 antibody.

In some embodiments, there is provided an antibody heavy chain comprising a polypeptide comprising, from N-terminus to C-terminus, a MM, a NCL, and a TBM, wherein the MM comprises an amino acid sequence selected from the group consisting of SEQ ID NOs: 35, 417, and 597-599; and wherein the TBM comprises a VH of an anti-CD3 antibody.

In some embodiments, there is provided a masked antibody targeting CD3 comprising a first polypeptide comprising, from N-terminus to C-terminus, a MM, a NCL, and a TBM, wherein the MM comprises an amino acid sequence selected from the group consisting of SEQ ID NOs: 35, 417, and 597-599, wherein the MM competes with CD3 to specifically bind the CD3-binding moiety; wherein the TBM comprises a VL, and the masked antibody further comprises a second polypeptide comprising a VH; and wherein the masked antibody binds CD3 via the VH and VL.

In some embodiments, there is provided a masked antibody targeting CD3 comprising a first polypeptide comprising, from N-terminus to C-terminus, a MM, a NCL, and a TBM, wherein the MM comprises an amino acid sequence selected from the group consisting of SEQ ID NOs: 35, 417, and 597-599, wherein the MM competes with CD3 to specifically bind the CD3-binding moiety; wherein the TBM comprises a VH, and the masked antibody further comprises a second polypeptide comprising a VL; and wherein the masked antibody binds CD3 via the VH and VL.

In some embodiments, there is provided a masked antibody targeting CD3 comprising a first polypeptide comprising, from N-terminus to C-terminus, a MM, a NCL, and a scFv of an anti-CD3 antibody, wherein the MM comprises an amino acid sequence selected from the group consisting of SEQ ID NOs: 35, 417, and 597-599, wherein the MM competes with CD3 to specifically bind the CD3-binding moiety; and wherein the masked antibody binds CD3 via the scFv.

In some embodiments, there is provided a masked antibody targeting CD3 comprising, from N-terminus to C-terminus, a masking moiety (MM), a NCL, and a CD3-binding moiety, wherein: a) the CD3-binding moiety comprises a VL and the masked antibody further comprises a second polypeptide comprising a VH; b) the CD3-binding moiety comprises a VH and the masked antibody further comprises a second polypeptide comprising a VL; c) the CD3-binding moiety comprises from the N-terminus to the C-terminus, a VL and a VH; or d) the CD3-binding moiety comprise from the N-terminus to the C-terminus, a VH and a VL; wherein the MM competes with CD3 to specifically bind the CD3-binding moiety; wherein the masked antibody binds CD3 via the VH and the VL; and wherein the masked antibody binds CD3 with half-maximal binding at a concentration of antibody (EC₅₀) that is at least 10 nM (e.g., at least 50 nM, or at least 100 nM) as determined by an enzyme-linked immunosorbent assay (ELISA). In some embodiments, the first antigen-binding fragment binds CD3 with a dissociation constant (Kd) of at least 50 nM. In some embodiments, the MM comprises an amino acid sequence selected from the group consisting of SEQ ID NOs: 35, 417, and 597-599.

Any one of the anti-CD3 antibodies and antigen-binding fragments that competitively bind to the same epitope as SP34, including anti-CD3 antibodies described in Section i) “Anti-CD3 antibody” and Tables 5B-5H may be used.

In some embodiments, the TBM (i.e., CD3 binding moiety) comprises a VH comprising a CDR-H1 comprising the amino acid sequence of SEQ ID NO: 61, a CDR-H2 comprising the amino acid sequence of SEQ ID NO: 62, and a CDR-H3 comprising the amino acid sequence of SEQ ID NO: 63; and/or a VL comprising a CDR-L1 comprising the amino acid sequence of SEQ ID NO: 64, a CDR-L2 comprising the amino acid sequence of SEQ ID NO: 65, and a CDR-L3 comprising the amino acid sequence of SEQ ID NO: 66. In some embodiments, the CD3 binding moiety comprises a VH comprising the amino acid sequence of SEQ ID NO: 67, and/or a VL comprising the amino acid sequence of SEQ ID NO: 68. In some embodiments, the CD3 binding moiety is a scFv comprising the amino acid sequence of SEQ ID NO: 79. In some embodiments, the MM comprises an amino acid sequence selected from the group consisting of SEQ ID NOs: 35, 417 and 597-599.

In some embodiments, the TBM (i.e., CD3 binding moiety) comprises a VH comprising a CDR-H1 comprising the amino acid sequence of SEQ ID NO: 390, a CDR-H2 comprising the amino acid sequence of SEQ ID NO: 392, and a CDR-H3 comprising the amino acid sequence of SEQ ID NO: 395; and/or a VL comprising a CDR-L1 comprising the amino acid sequence of SEQ ID NO: 397, a CDR-L2 comprising the amino acid sequence of SEQ ID NO: 398, and a CDR-L3 comprising the amino acid sequence of SEQ ID NO: 400. In some embodiments, the CD3 binding moiety comprises a VH comprising the amino acid sequence of SEQ ID NO: 402, and/or a VL comprising the amino acid sequence of SEQ ID NO: 403. In some embodiments, the CD3 binding moiety is a scFv comprising the amino acid sequence of SEQ ID NO: 421. In some embodiments, the MM comprises an amino acid sequence selected from the group consisting of SEQ ID NOs: 35, 417 and 597-599.

In some embodiments, the TBM (i.e., CD3 binding moiety) comprises a VH comprising a CDR-H1 comprising the amino acid sequence of SEQ ID NO: 390, a CDR-H2 comprising the amino acid sequence of SEQ ID NO: 394, and a CDR-H3 comprising the amino acid sequence of SEQ ID NO: 395; and/or a VL comprising a CDR-L1 comprising the amino acid sequence of SEQ ID NO: 397, a CDR-L2 comprising the amino acid sequence of SEQ ID NO: 380, and a CDR-L3 comprising the amino acid sequence of SEQ ID NO: 381. In some embodiments, the CD3 binding moiety comprises a VH comprising the amino acid sequence of SEQ ID NO: 410, and/or a VL comprising the amino acid sequence of SEQ ID NO: 411. In some embodiments, the CD3 binding moiety is a scFv comprising the amino acid sequence of SEQ ID NO: 422. In some embodiments, the MM comprises an amino acid sequence selected from the group consisting of SEQ ID NOs: 35, 417 and 597-599.

Any one of the masking moieties for anti-CD3 antibodies described herein may be used, including, for example, the masking moieties of section F, “Masking Moiety (MM)” and Tables B, 18-22, 13A and 40. In some embodiments, the MM comprises the amino acid sequence of SEQ ID NO: 417. In some embodiments, the MM comprises the amino acid sequence of SEQ ID NO: 35. In some embodiments, the MM comprises the amino acid sequence of SEQ ID NO: 597. In some embodiments, the MM comprises the amino acid sequence of SEQ ID NO: 598. In some embodiments, the MM comprises the amino acid sequence of SEQ ID NO: 599.

Any one of the non-cleavable linkers (NCLs) described herein may be used, including, for example, the cleavable moieties of section I, “Linker.”

In some embodiments, the masked antibody targeting CD3 is a multispecific antibody, such as a bispecific antibody. In some embodiments, the masked antibody targeting CD3 is a bispecific T cell engager (BiTE) molecule, which also targets a tumor antigen, such as HER2 or CD3.

In some embodiments, the masked antibody comprises a light chain comprising an amino acid sequence of TY23105, TY23110, TY23115, or TY23118, as shown in Table 3D. In some embodiments, the masked antibody comprises a light chain comprising an amino acid sequence selected from the group consisting of SEQ ID NOs: 589, 591, 593, and 595. In some embodiments, the masked antibody comprises a heavy chain comprising an amino acid sequence of TY23105, TY23110, TY23115, or TY23118, as shown in Table 3D. In some embodiments, the masked antibody comprises a heavy chain comprising an amino acid sequence selected from the group consisting of SEQ ID NOs: 590, 592, 594, and 596.

b) Masked Anti-CD3 AntiBodies Derived from OKT3

Also provided herein are masked antibodies, masked antibody fragments, and polypeptides that target CD3, comprising a masking moiety (MM) comprising an amino acid sequence according to Formula (X): X₁X₂X₃DX₄X₅CX₆X₇DX₈X₉X₁₀CX₁₁X₁₂ (SEQ ID NO: 669), wherein X₁ is A or D, X₂ is A, D, or P, X₃ is D, H, or P, X₄ is F or P, X₅ is D or P, X₆ is D or P, X₇ is A or P, X₈ is D, N, or P, X₉ is A, N, or P, X₁₀ is D, H, or S, X₁₁ is H, P, or Y, and X₁₂ is N, P, or Y. In some embodiments, the masked antibody comprises a MM comprising an amino acid sequence selected from the group consisting of SEQ ID NO: 585-588. In some embodiments, the MM comprises the amino acid of SEQ ID NO: 585. In some embodiments, the MM comprises the amino acid of SEQ ID NO: 586. In some embodiments, the MM comprises the amino acid of SEQ ID NO: 587. In some embodiments, the MM comprises the amino acid of SEQ ID NO: 588.

In some embodiments, there is provided an antibody light chain comprising a polypeptide comprising, from N-terminus to C-terminus, a MM, a non-cleavable linker (NCL), and a target binding moiety (TBM), wherein the MM comprises an amino acid sequence selected from the group consisting of SEQ ID NO: 585-588; and wherein the TBM comprises a VL of an anti-CD3 antibody.

In some embodiments, there is provided an antibody heavy chain comprising a polypeptide comprising, from N-terminus to C-terminus, a MM, a NCL, and a TBM, wherein the MM comprises an amino acid sequence selected from the group consisting of SEQ ID NO: 585-588; and wherein the TBM comprises a VH of an anti-CD3 antibody.

In some embodiments, there is provided a masked antibody targeting CD3 comprising a first polypeptide comprising, from N-terminus to C-terminus, a MM, a NCL, and a TBM, wherein the MM comprises an amino acid sequence selected from the group consisting of SEQ ID NO: 585-588, wherein the MM competes with CD3 to specifically bind the CD3-binding moiety; wherein the TBM comprises a VL, and the masked antibody further comprises a second polypeptide comprising a VH; and wherein the masked antibody binds CD3 via the VH and VL.

Any one of the anti-CD3 antibodies and antigen-binding fragments that competitively bind to the same epitope as OKT3, including anti-CD3 antibodies described in Table 3B may be used.

In some embodiments, there is provided a masked antibody targeting CD3 comprising a first polypeptide comprising, from N-terminus to C-terminus, a MM, a NCL, and a TBM, wherein the MM comprises an amino acid sequence selected from the group consisting of SEQ ID NO: 585-588, wherein the MM competes with CD3 to specifically bind the CD3-binding moiety; wherein the TBM comprises a VH, and the masked antibody further comprises a second polypeptide comprising a VL; and wherein the masked antibody binds CD3 via the VH and VL.

In some embodiments, there is provided a masked antibody targeting CD3 comprising a first polypeptide comprising, from N-terminus to C-terminus, a MM, a NCL, and a scFv of an anti-CD3 antibody, wherein the MM comprises an amino acid sequence selected from the group consisting of SEQ ID NO: 585-588, wherein the MM competes with CD3 to specifically bind the CD3-binding moiety; and wherein the masked antibody binds CD3 via the scFv.

In some embodiments, a masked antibody is provided comprising, from N-terminus to C-terminus, a masking moiety (MM), a NCL, and a CD3-binding moiety, wherein: a) the CD3-binding moiety comprises a VL and the masked antibody further comprises a second polypeptide comprising a VH; b) the CD3-binding moiety comprises a VH and the masked antibody further comprises a second polypeptide comprising a VL; c) the CD3-binding moiety comprises from the N-terminus to the C-terminus, a VL and a VH; or d) the CD3-binding moiety comprises from the N-terminus to the C-terminus, a VH and a VL; and wherein the MM competes with CD3 to specifically bind the CD3-binding moiety; wherein the masked antibody binds CD3 via the VH and the VL; wherein the MM comprises an amino acid sequence selected from the group consisting of SEQ ID NO: 585-588.

In some embodiments, the anti-CD3 antigen-binding fragment is selected from the group consisting of a Fab, a Fv, a scFab and a scFv.

In some embodiments, the TBM (i.e., CD3 binding moiety) comprises a VH comprising a CDR-H1 comprising the amino acid sequence of SEQ ID NO: 368, a CDR-H2 comprising the amino acid sequence of SEQ ID NO: 369, and a CDR-H3 comprising the amino acid sequence of SEQ ID NO: 370; and/or a VL comprising a CDR-L1 comprising the amino acid sequence of SEQ ID NO: 371, a CDR-L2 comprising the amino acid sequence of SEQ ID NO: 372, and a CDR-L3 comprising the amino acid sequence of SEQ ID NO: 373. In some embodiments, the CD3 binding moiety comprises a VH comprising the amino acid sequence of SEQ ID NO: 366, and/or a VL comprising the amino acid sequence of SEQ ID NO: 367. In some embodiments, the MM comprises an amino acid sequence selected from the group consisting of SEQ ID NOs: 585-588.

Any one of the non-cleavable linkers (NCLs) described herein may be used, including, for example, the cleavable moieties of section I, “Linker.”

In some embodiments, the masked antibody comprises a light chain comprising an amino acid sequence of TY23100, TY23101, TY23102, or TY23104, as shown in Table 3C. In some embodiments, the masked antibody comprises a light chain comprising an amino acid sequence selected from the group consisting of SEQ ID NOs: 577, 579, 581, and 583. In some embodiments, the masked antibody comprises a heavy chain comprising an amino acid sequence of TY23100, TY23101, TY23102, or TY23104, as shown in Table 3C. In some embodiments, the masked antibody comprises a heavy chain comprising an amino acid sequence selected from the group consisting of SEQ ID NOs: 578, 580, 582, and 584.

ii. Masked Anti-HER2 Antibodies

The present application provides masked antibodies, masked antibody fragments, and polypeptides that target HER2, comprising a masking moiety (MM) comprising an amino acid sequence selected from the group consisting of SEQ ID NOs: 36, 419, 432-476, and 491-515. In some embodiments, the MM comprises the amino acid sequence of SEQ ID NO: 36 or 419.

In some embodiments, there is provided an antibody light chain comprising a polypeptide comprising, from N-terminus to C-terminus, a MM, a non-cleavable linker (NCL), and a target binding moiety (TBM), wherein the MM comprises an amino acid sequence selected from the group consisting of SEQ ID NOs: 36, 419, 432-476, and 491-515; and wherein the TBM comprises a VL of an anti-HER2 antibody. In some embodiments, the MM comprises the amino acid sequence of SEQ ID NO: 36 or 419.

In some embodiments, there is provided an antibody heavy chain comprising a polypeptide comprising, from N-terminus to C-terminus, a MM, a NCL, and a TBM, wherein the MM comprises an amino acid sequence selected from the group consisting of SEQ ID NOs: 36, 419, 432-476, and 491-515; and wherein the TBM comprises a VH of an anti-HER2 antibody. In some embodiments, the MM comprises the amino acid sequence of SEQ ID NO: 36 or 419.

In some embodiments, there is provided a masked antibody targeting HER2 comprising a first polypeptide comprising, from N-terminus to C-terminus, a MM, a NCL, and a TBM, wherein the MM comprises an amino acid sequence selected from the group consisting of SEQ ID NOs: 36, 419, 432-476, and 491-515, wherein the MM competes with HER2 to specifically bind the HER2-binding moiety; wherein the TBM comprises a VL, and the masked antibody further comprises a second polypeptide comprising a VH; and wherein the masked antibody binds HER2 via the VH and VL. In some embodiments, the MM comprises the amino acid sequence of SEQ ID NO: 36 or 419.

In some embodiments, there is provided a masked antibody targeting HER2 comprising a first polypeptide comprising, from N-terminus to C-terminus, a MM, a NCL, and a TBM, wherein the MM comprises an amino acid sequence selected from the group consisting of SEQ ID NOs: 36, 419, 432-476, and 491-515, wherein the MM competes with HER2 to specifically bind the HER2-binding moiety; wherein the TBM comprises a VH, and the masked antibody further comprises a second polypeptide comprising a VL; and wherein the masked antibody binds HER2 via the VH and VL. In some embodiments, the MM comprises the amino acid sequence of SEQ ID NO: 36 or 419.

In some embodiments, there is provided a masked antibody targeting HER2 comprising a first polypeptide comprising, from N-terminus to C-terminus, a MM, a NCL, and a scFv and an anti-HER2 antibody, wherein the MM comprises an amino acid sequence selected from the group consisting of SEQ ID NOs: 36, 419, 432-476, and 491-515, wherein the MM competes with HER2 to specifically bind the HER2-binding moiety; and wherein the masked antibody binds HER2 via the scFv. In some embodiments, the MM comprises the amino acid sequence of SEQ ID NO: 36 or 419.

Any one of the non-cleavable linkers (NCLs) described herein may be used, including, for example, the cleavable moieties of section I, “Linker.”

In some embodiments, the TBM (i.e., HER2-binding moiety) comprises a VH comprising an CDR-H1 comprising the amino acid sequence of SEQ ID NO: 69, a CDR-H2 comprising the amino acid sequence of SEQ ID NO: 70, and a CDR-H3 comprising the amino acid sequence of SEQ ID NO: 71; and/or a VL comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74.

In some embodiments, the TBM (i.e., HER2-binding moiety) comprises a VH comprising an CDR-H1 comprising the amino acid sequence of SEQ ID NO: 69 or a variant thereof comprising up to about 3 (such as about any of 1, 2, or 3) amino acid substitutions, a CDR-H2 comprising the amino acid sequence of SEQ ID NO: 70 or a variant thereof comprising up to about 3 (such as about any of 1, 2, or 3) amino acid substitutions, and a CDR-H3 comprising the amino acid sequence of SEQ ID NO: 71 or a variant thereof comprising up to about 3 (such as about any of 1, 2, or 3) amino acid substitutions; and/or a VL comprising a CDR-L1 comprising the amino acid sequence of SEQ ID NO: 72 or a variant thereof comprising up to about 3 (such as about any of 1, 2, or 3) amino acid substitutions, a CDR-L2 comprising the amino acid sequence of SEQ ID NO: 73 or a variant thereof comprising up to about 3 (such as about any of 1, 2, or 3) amino acid substitutions, and a CDR-L3 comprising the amino acid sequence of SEQ ID NO: 74 or a variant thereof comprising up to about 3 (such as about any of 1, 2, or 3) amino acid substitutions.

In some embodiments, the TBM (i.e., HER2-binding moiety) comprises a VH comprising an CDR-H1 comprising the amino acid sequence of SEQ ID NO: 423, a CDR-H2 comprising the amino acid sequence of SEQ ID NO: 424, and a CDR-H3 comprising the amino acid sequence of SEQ ID NO: 71; and/or a VL comprising a CDR-L1 comprising the amino acid sequence of SEQ ID NO: 72, a CDR-L2 comprising the amino acid sequence of SEQ ID NO: 73, and a CDR-L3 comprising the amino acid sequence of SEQ ID NO: 74.

In some embodiments, the TBM (i.e., HER2-binding moiety) comprises a VH comprising an CDR-H1 comprising the amino acid sequence of SEQ ID NO: 423 or a variant thereof comprising up to about 3 (such as about any of 1, 2, or 3) amino acid substitutions, a CDR-H2 comprising the amino acid sequence of SEQ ID NO: 424 or a variant thereof comprising up to about 3 (such as about any of 1, 2, or 3) amino acid substitutions, and a CDR-H3 comprising the amino acid sequence of SEQ ID NO: 71 or a variant thereof comprising up to about 3 (such as about any of 1, 2, or 3) amino acid substitutions; and/or a VL comprising a CDR-L1 comprising the amino acid sequence of SEQ ID NO: 72 or a variant thereof comprising up to about 3 (such as about any of 1, 2, or 3) amino acid substitutions, a CDR-L2 comprising the amino acid sequence of SEQ ID NO: 73 or a variant thereof comprising up to about 3 (such as about any of 1, 2, or 3) amino acid substitutions, and a CDR-L3 comprising the amino acid sequence of SEQ ID NO: 74 or a variant thereof comprising up to about 3 (such as about any of 1, 2, or 3) amino acid substitutions.

In some embodiments, the TBM (i.e., HER2-binding moiety) comprises a VH comprising the amino acid sequence of SEQ ID NO: 75 and/or a VL comprising the amino acid sequence of SEQ ID NO: 76.

In some embodiments, the TBM (i.e., HER2-binding moiety) comprises a VH comprising an amino acid sequence comprising at least 80% (e.g., at least any one of 85%, 87%, 90%, 91%, 92%, 93%, 94%, 95%, 96%, 97%, 98%, 99%, or more) sequence identity to the amino acid sequence of SEQ ID NO: 75 and/or a VL comprising an amino acid sequence comprising at least 80% (e.g., at least any one of 85%, 87%, 90%, 91%, 92%, 93%, 94%, 95%, 96%, 97%, 98%, 99%, or more) sequence identity to the amino acid sequence of SEQ ID NO: 76.

In some embodiments, the masked antibody targeting HER2 is a multispecific antibody, such as a bispecific antibody. In some embodiments, the masked antibody targeting HER2 is a bispecific T cell engager (BiTE) molecule, which also targets CD3.

### III. Variants and Derivatives

Also contemplated herein are variants and derivatives of any one of the multispecific antibodies, masked antibodies, activatable antibodies, isolated anti-CD3 antibodies or antigen-binding fragments thereof, and activatable multispecific antibodies described herein.

In some embodiments, the antibody (e.g., the multispecific and/or activatable antibody) derivative is derived from modifications of the amino acid sequences of the parent antibody while conserving the overall molecular structure of the parent antibody. Amino acid sequences of any regions of the parent antibody chains may be modified, such as framework regions, CDR regions, or constant regions. Types of modifications include substitutions, insertions, deletions, or combinations thereof, of one or more amino acids of the parent antibody.

In some embodiments, the antibody (e.g., the multispecific and/or activatable antibody) derivative comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence of an antibody described herein. In some embodiments, the antibody (e.g., activatable antibody) derivative comprises 1, 2, or 3 conservative or non-conservative substitutions in any one of CDR-H1, CDR-H2, CDR-H3, CDR-L1, CDR-L2 and/or CDR-L3 of an antibody described herein. In some embodiments, the antibody (e.g., the multispecific and/or activatable antibody) derivative comprises 1, 2, 3, 4, 5, 6, 7, 8, 9, 10, 11, 12, 13, 14, or 15 conservative or non-conservative substitutions, additions and/or deletions to the VH and/or VL of an antibody described herein. In some embodiments, the antibody (e.g., the multispecific and/or activatable antibody) derivative comprises a sequence having at least 80% (e.g., at least any one of 85%, 87%, 90%, 91%, 92%, 93%, 94%, 95%, 96%, 97%, 98%, 99%, or more) identity to a VH and/or VL sequence of an antibody described herein.

Amino acid substitutions encompass both conservative substitutions and non-conservative substitutions. The term “conservative amino acid substitution” means a replacement of one amino acid with another amino acid where the two amino acids have similarity in certain physico-chemical properties such as polarity, charge, solubility, hydrophobicity, hydrophilicity, and/or the amphipathic nature of the residues involved. For example, substitutions typically may be made within each of the following groups: (a) nonpolar (hydrophobic) amino acids, such as alanine, leucine, isoleucine, valine, proline, phenylalanine, tryptophan, and methionine; (b) polar neutral amino acids, such as glycine, serine, threonine, cysteine, tyrosine, asparagine, and glutamine; (c) positively charged (basic) amino acids, such as arginine, lysine, and histidine; and (d) negatively charged (acidic) amino acids, such as aspartic acid and glutamic acid.

The modifications may be made in any positions of the amino acid sequences of an antibody, including the CDRs, framework regions, or constant regions. In some embodiments, the present application provides an antibody derivative that contains the VH and VL CDR sequences of an illustrative antibody described herein, yet contains framework sequences different from those of the illustrative antibody. Such framework sequences can be obtained from public DNA databases or published references that include germline antibody gene sequences. For example, germline DNA sequences for human heavy and light chain variable region genes can be found in the Genbank database or in the “VBase” human germline sequence database (Kabat et al., Sequences of Proteins of Immunological Interest, Fifth Edition, U.S. Department of Health and Human Services, NIH Publication No. 91-3242 (1991); Tomlinson et al., J. Mal. Biol. 227:776-798 (1992); and Cox et al., Eur. J. Immunol. 24:827-836 (1994)). Framework sequences that may be used in constructing an antibody derivative include those that are structurally similar to the framework sequences used by illustrative antibodies of the application For example, the CDR-H1, CDR-H2, and CDR-H3 sequences, and the CDR-L1, CDR-L2, and CDR-L3 sequences of an illustrative antibody can be grafted onto framework regions that have the identical sequence as that found in the germline immunoglobulin gene from which the framework sequence derive, or the CDR sequences can be grafted onto framework regions that contain one or more mutations as compared to the germline sequences.

In some embodiments, the antibody derivative is a chimeric antibody, which comprises an amino acid sequence of an illustrative antibody described herein. In one example, one or more CDRs from one or more illustrative antibodies are combined with CDRs from an antibody from a non-human animal, such as mouse or rat. In another example, all of the CDRs of the chimeric antibody are derived from one or more illustrative antibodies. In some particular embodiments, the chimeric antibody comprises one, two, or three CDRs from the heavy chain variable region and/or one, two, or three CDRs from the light chain variable region of an illustrative antibody. Chimeric antibodies can be generated using conventional methods known in the art.

Another type of modification is to mutate amino acid residues within the CDR regions of the VH and/or VL chain. Site-directed mutagenesis or PCR-mediated mutagenesis can be performed to introduce the mutation(s) and the effect on antibody binding, or other functional property of interest, can be evaluated in in vitro or in vivo assays known in the art. Typically, conservative substitutions are introduced. The mutations may be amino acid additions and/or deletions. Moreover, typically no more than one, two, three, four or five residues within a CDR region are altered. In some embodiments, the antibody derivative comprises 1, 2, 3, or 4 amino acid substitutions in the heavy chain CDRs and/or in the light chain CDRs. In another embodiment, the amino acid substitution is to change one or more cysteines in an antibody to another residue, such as, without limitation, alanine or serine. The cysteine may be a canonical or non-canonical cysteine. In some embodiments, the antibody derivative has 1, 2, 3, or 4 conservative amino acid substitutions in the heavy chain CDR regions relative to the amino acid sequences of an illustrative antibody.

Modifications may also be made to the framework residues within the VH and/or VL regions. Typically, such framework variants are made to decrease the immunogenicity of the antibody. One approach is to “back mutate” one or more framework residues to the corresponding germline sequence. An antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back mutated” to the germline sequence by, for example, site-directed mutagenesis or PCR-mediated mutagenesis.

In addition, modifications may also be made within the Fc region of an illustrative antibody, typically to alter one or more functional properties of the antibody, such as serum half-life, complement fixation, Fc receptor binding, and/or antigen-dependent cellular cytotoxicity. In one example, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody. In another case, the Fc hinge region of an antibody is mutated to decrease the biological half-life of the antibody.

In some embodiments, the Fc region of the antibody (e.g., the multispecific and/or activatable antibody) described herein has at least one (e.g., at least one, two or three or more) amino acid substitution in addition to the amino acid substitutions that form engineered disulfide bonds or salt bridges as described herein compared to the Fc region of a wild-type IgG or a wild-type antibody. In some embodiments, the Fc region has at least 80%, at least 85%, at least 90%, at least 95% or more homology with a native sequence Fc region and/or with an Fc region of a parent polypeptide.

Furthermore, the Fc region may be modified to alter its potential glycosylation site or pattern in accordance with routine experimentation known in the art. In another aspect, the present application provides a derivative of an antibody (e.g., the multispecific and/or activatable antibody) described herein that contains at least one mutation in a variable region of a light chain or heavy chain that changes the pattern of glycosylation in the variable region. Such an antibody derivative may have an increased affinity and/or a modified specificity for binding an antigen. The mutations may add a novel glycosylation site in the V region, change the location of one or more V region glycosylation site(s), or remove a pre-existing V region glycosylation site. In some embodiments, the present application provides a derivative of an antibody described herein having a potential N-linked glycosylation site at asparagine in the heavy chain variable region, wherein the potential N-linked glycosylation site in one heavy chain variable region is removed. In some embodiments, the present application provides a derivative of an antibody described herein having a potential N-linked glycosylation site at asparagine in the heavy chain variable region, wherein the potential N-linked glycosylation site in both heavy chain variable regions is removed. Method of altering the glycosylation pattern of an antibody is known in the art, such as those described in U.S. Pat. No. 6,933,368, the application of which incorporated herein by reference.

In some embodiments, the antibodies described herein (e.g., multispecific antibodies and/or activatable antibodies) may be in any class, such as IgG, IgM, IgE, IgA, or IgD. In some embodiments, the activatable antibodies described herein (e.g., a CD3 and/or HER2 antibody) are in the IgG class, such as IgG1, IgG2, IgG3, or IgG4 subclass. An antibody described herein antibody can be converted from one class or subclass to another class or subclass using methods known in the art. An exemplary method for producing an antibody in a desired class or subclass comprises the steps of isolating a nucleic acid encoding a heavy chain of an antibody described herein (e.g., multispecific and/or activatable antibody) and a nucleic acid encoding a light chain of an antibody described herein (e.g., multispecific and/or activatable antibody), isolating the sequence encoding the VH region, ligating the VH sequence to a sequence encoding a heavy chain constant region of the desired class or subclass, expressing the light chain gene and the heavy chain construct in a cell, and collecting the antibody.

Antibody variants are also provided with amino-terminal leader extensions. For example, one or more amino acid residues of the amino-terminal leader sequence are present at the amino-terminus of any one or more heavy or light chains of an antibody.

The antibodies (e.g., multispecific and/or activatable antibodies) described herein may be further modified. In some embodiments, the antibody is linked to an additional molecular entity. Examples of additional molecular entities include pharmaceutical agents, peptides or proteins, detection agent or labels, and antibodies.

In some embodiments, an antibody (e.g., a multispecific and/or activatable antibody) of the present application is linked to a pharmaceutical agent. Examples of pharmaceutical agents include cytotoxic agents or other cancer therapeutic agents, and radioactive isotopes. Specific examples of cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Examples of radioactive isotopes that can be conjugated to antibodies for use diagnostically or therapeutically include, but are not limited to, iodine131, indiumrn, yttrium90 and lutetium 177. Methods for linking a polypeptide to a pharmaceutical agent are known in the art, such as using various linker technologies. Examples of linker types include hydrazones, thioethers, esters, disulfides and peptide-containing linkers. For further discussion of linkers and methods for linking therapeutic agents to antibodies see e.g., Saito et al., Adv. Drug De/iv. Rev. 55:199-215 (2003); Trail, et al., Cancer Immunol. Immunother. 52:328-337 (2003); Payne, Cancer Cell 3:207-212 (2003); Allen, Nat. Rev. Cancer 2:750-763 (2002); Pastan and Kreitman, Curr. Opin. Investig. Drugs 3: 1089-1091 (2002); Senter and Springer (2001) Adv. Drug De/iv. Rev. 53:247-264.

In some embodiments, an antibody (e.g., a multispecific and/or activatable antibody) of the present application is conjugated to a label and/or a cytotoxic agent. As used herein, a label is a moiety that facilitates detection of the antibody and/or facilitates detection of a molecule to which the antibody binds. Nonlimiting exemplary labels include, but are not limited to, radioisotopes, fluorescent groups, enzymatic groups, chemiluminescent groups, biotin, epitope tags, metal-binding tags, etc. One skilled in the art can select a suitable label according to the intended application.

As used herein, a cytotoxic agent is a moiety that reduces the proliferative capacity of one or more cells. A cell has reduced proliferative capacity when the cell becomes less able to proliferate, for example, because the cell undergoes apoptosis or otherwise dies, the cell fails to proceed through the cell cycle and/or fails to divide, the cell differentiates, etc. Nonlimiting exemplary cytotoxic agents include, but are not limited to, radioisotopes, toxins, and chemotherapeutic agents. One skilled in the art can select a suitable cytotoxic according to the intended application.

In some embodiments, a label and/or a cytotoxic agent is conjugated to an antibody using chemical methods in vitro. Nonlimiting exemplary chemical methods of conjugation are known in the art, and include services, methods and/or reagents commercially available from, e.g., Thermo Scientific Life Science Research Produces (formerly Pierce; Rockford, Ill.), Prozyme (Hayward, Calif.), SACRI Antibody Services (Calgary, Canada), AbD Serotec (Raleigh, N.C.), etc. In some embodiments, when a label and/or cytotoxic agent is a polypeptide, the label and/or cytotoxic agent can be expressed from the same expression vector with at least one antibody chain to produce a polypeptide comprising the label and/or cytotoxic agent fused to an antibody chain. One skilled in the art can select a suitable method for conjugating a label and/or cytotoxic agent to an antibody according to the intended application.

### IV. Methods of Preparation

In one aspect, the present application provides methods for preparing the multispecific antibodies, masked antibodies, activatable antibodies, isolated anti-CD3 antibodies or antigen-binding fragments thereof, and activatable multispecific antibodies described herein. For example, methods for preparing a multispecific antibody, masked antibody, activatable antibody, isolated anti-CD3 antibody or antigen-binding fragment thereof, or activatable multispecific antibody comprises culturing a host cell comprising one or more nucleic acid(s) or vector(s) that encode the multispecific antibody, masked antibody, activatable antibody, anti-CD3 antibody or antigen-binding fragment thereof, or activatable multispecific antibody under conditions that allow expression of the nucleic acid(s) or vector, and recovering the multispecific antibody, masked antibody, activatable antibody, anti-CD3 antibody or antigen-binding fragment thereof, or activatable multispecific antibody from the host cell culture are provided.

Polypeptides (e.g., any of the multispecific antibodies, masked antibodies, activatable antibodies, isolated anti-CD3 antibodies or antigen-binding fragments thereof, and activatable multispecific antibodies described herein) of the present application may be produced using recombinant methods and compositions, e.g., as described in U.S. Pat. No. 4,816,567. In some embodiments, isolated nucleic acids encoding any or the polypeptides (e.g., any of the multispecific antibodies, masked antibodies, activatable antibodies, isolated anti-CD3 antibodies or antigen-binding fragments thereof, and activatable multispecific antibodies described herein) are provided. In some embodiments, there is provided one or more nucleic acids encoding an isolated antibody, or antigen-binding fragment thereof, that binds human CD3. In some embodiments, there is provided one or more nucleic acids encoding an amino acid sequence comprising the VL(s) and/or an amino acid sequence comprising the VH(s) of the multispecific antibodies or activatable antibodies (e.g., the light and/or heavy chains of the antibodies). In some embodiments, one or more vectors (e.g., expression vectors) comprising such nucleic acids are provided herein. In some embodiments, a host cell comprising (e.g., has been transformed with) one or more vectors comprising nucleic acid(s) encoding the multispecific antibody, masked antibody, activatable antibody, anti-CD3 antibody or antigen-binding fragment thereof, or activatable multispecific antibody described herein. In some embodiments, the host cell is eukaryotic, e.g. a yeast cell, an insect cell, a Chinese Hamster Ovary (CHO) cell or lymphoid cell (e.g., YO, NS0, Sp20 cell).

For recombinant production of polypeptides (e.g., any of the multispecific antibodies, masked antibodies, activatable antibodies, isolated anti-CD3 antibodies or antigen-binding fragments thereof, and activatable multispecific antibodies described herein) of the present application, nucleic acid encoding a polypeptide (e.g., a multispecific antibody, masked antibody, activatable antibody, anti-CD3 antibody or antigen-binding fragment thereof, or activatable multispecific antibody described herein), e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the polypeptide(s)).

Suitable host cells for cloning or expression of polypeptide-encoding vectors include prokaryotic or eukaryotic cells. For example, polypeptides may be produced in bacteria, in particular when glycosylation and Fc effector function are not needed (see, e.g., U.S. Pat. Nos. 5,648,237, 5,789,199, and 5,840,523; See also Charlton, Methods in Molecular Biology, Vol. 248 (B. K. C. Lo, ed., Humana Press, Totowa, NJ, 2003), pp. 245-254, describing expression of antibody fragments in E. coli.). After expression, the polypeptide may be isolated from the bacterial cell paste in a soluble fraction and may be further purified.

In addition to prokaryotes, eukaryotic microbes such as filamentous fungi or yeast are suitable cloning or expression hosts for polypeptide-encoding vectors, including fungi and yeast strains whose glycosylation pathways have been “humanized,” resulting in the production of a polypeptide with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

In order for some secreted proteins to express and secrete in large quantities, a leader sequence from a heterologous protein may be desirable. In some embodiments, employing heterologous leader sequences may be advantageous in that a resulting mature polypeptide may remain unaltered as the leader sequence is removed in the ER during the secretion process. The addition of a heterologous leader sequence may be required to express and secrete some proteins.

Certain exemplary leader sequence sequences are described, e.g., in the online Leader sequence Database maintained by the Department of Biochemistry, National University of Singapore. See Choo et al., BMC Bioinformatics, 6: 249 (2005); and PCT Publication No. WO 2006/081430.

### V. Compositions and Kits

In some embodiments, the present application provides pharmaceutical compositions comprising any one of the multispecific antibodies (e.g., masked multispecific antibodies, including activatable multispecific antibodies), activatable antibodies (e.g., activatable anti-CD3 antibodies or activatable anti-HER2 antibodies), isolated anti-CD3 antibodies or antigen-binding fragments thereof, and activatable multispecific antibodies (e.g., activatable HER2xCD3 antibodies, activatable CD20xCD3 antibodies, or activatable TROP2xCD3 antibodies) described herein, and a pharmaceutically acceptable carrier. The compositions can be prepared by conventional methods known in the art.

The term “pharmaceutically acceptable carrier” refers to any inactive substance that is suitable for use in a formulation for the delivery of a polypeptide (e.g., an activatable antibody, isolated anti-CD3 antibody or antigen-binding fragment thereof, or activatable and/or multispecific antibody). A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride.

The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering a polypeptide (e.g., an activatable antibody, isolated anti-CD3 antibody or antigen-binding fragment thereof, or activatable multispecific antibody) is a sterile liquid, such as a solution, suspension, or dispersion, for injection or infusion. Sterile solutions can be prepared by incorporating the polypeptide (e.g., an activatable antibody, isolated anti-CD3 antibody or antigen-binding fragment thereof, or activatable multispecific antibody) in the required amount in an appropriate carrier, followed by sterilization microfiltration. Dispersions may be prepared by incorporating the polypeptide into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

The relative amount of a polypeptide (e.g., an activatable antibody, isolated anti-CD3 antibody or antigen-binding fragment thereof, or activatable and/or multispecific antibody) included in the composition will vary depending upon a number of factors, such as the specific polypeptide and carriers used, dosage form, and desired release and pharmacodynamic characteristics. The amount of a (e.g., an activatable antibody, isolated anti-CD3 antibody or antigen-binding fragment thereof, or activatable and/or multispecific antibody) in a single dosage form will generally be that amount which produces a therapeutic effect, but may also be a lesser amount. Generally, this amount will range from about 0.01 percent to about 99 percent, from about 0.1 percent to about 70 percent, or from about 1 percent to about 30 percent relative to the total weight of the dosage form.

In addition to the polypeptide (e.g., an activatable antibody, isolated anti-CD3 antibody or antigen-binding fragment thereof, or activatable and/or multispecific antibody), one or more additional therapeutic agents may be included in the composition. The suitable amount of the additional therapeutic agent to be included in the composition can be readily selected by a person skilled in the art, and will vary depending on a number of factors, such as the particular agent and carriers used, dosage form, and desired release and pharmacodynamic characteristics. The amount of the additional therapeutic agent included in a single dosage form will generally be that amount of the agent, which produces a therapeutic effect, but may be a lesser amount as well.

Any of the polypeptides (e.g., an activatable antibody, isolated anti-CD3 antibody or antigen-binding fragment thereof, or activatable and/or multispecific antibody) and/or compositions (e.g., pharmaceutical compositions) described herein may be used in the preparation of a medicament (e.g., a medicament for use in treating or delaying progression of cancer in a subject in need thereof).

In some embodiments, provided herein is a kit comprising any one of the activatable antibodies, isolated anti-CD3 antibodies or antigen-binding fragments thereof, activatable and/or multispecific antibodies and/or compositions described herein. In some embodiments, the kit further comprises a package insert comprising instructions for use of the activatable antibodies, isolated anti-CD3 antibodies or antigen-binding fragments thereof, activatable and/or multispecific antibodies and/or compositions. The package insert may contain information about the indications, usage, dosage, administration, combination therapy, contraindications and/or warnings concerning the use of a therapeutic product. In some embodiments, the kit further comprises one or more buffers, e.g., for storing, transferring, administering, or otherwise using the activatable antibodies, isolated anti-CD3 antibodies or antigen-binding fragments thereof, activatable and/or multispecific antibodies and/or compositions. In some embodiments, the kit further comprises one or more containers for storing or administering (e.g., syringes, etc.) the activatable antibodies, isolated anti-CD3 antibodies or antigen-binding fragments thereof, activatable and/or multispecific antibodies and/or compositions. Also provided are articles of manufacture comprising any one of the multispecific antibodies, activatable antibodies, isolated anti-CD3 antibodies or antigen-binding fragments thereof, activatable multispecific antibodies and/or compositions described herein.

### VI. Methods of Treatment

The multispecific antibodies (e.g., masked multispecific antibodies, including activatable multispecific antibodies), activatable antibodies (e.g., activatable anti-CD3 antibodies, activatable anti-HER2 antibodies, or activatable TROP2xCD3 antibodies), isolated anti-CD3 antibodies or antigen-binding fragments thereof, activatable multispecific antibodies (e.g., activatable HER2xCD3 antibodies, activatable CD20xCD3 antibodies, or activatable TROP2xCD3 antibodies), and pharmaceutical compositions described herein are useful for therapeutic, diagnostic, or other purposes, such as modulating an immune response, treating cancer (e.g., solid or liquid cancer), enhancing efficacy of other cancer therapy, enhancing vaccine efficacy, or treating autoimmune diseases.

In some embodiments, there is provided a method for treating a disease or condition in a subject in need thereof comprising administering to the subject an effective amount of a pharmaceutical composition comprising any one of the multispecific antibodies (e.g., masked multispecific antibodies including activatable multispecific antibodies), activatable antibodies (e.g., activatable anti-CD3 antibodies, activatable anti-HER2 antibodies, or activatable TROP2xCD3 antibodies), isolated anti-CD3 antibodies or antigen-binding fragments thereof, and activatable multispecific antibodies (e.g., activatable HER2xCD3 antibodies, activatable CD20xCD3 antibodies, or activatable TROP2xCD3 antibodies) described herein. In some embodiments, the disease or condition is cancer. A variety of cancers may be treated or prevented with a method, use, or pharmaceutical composition provided by the present application.

In some embodiments, there is provided a method for treating a cancer in a subject in need thereof comprising administering to the subject an effective amount of a pharmaceutical composition comprising any one of the BiTE or activatable BiTE molecules (e.g., any one of the HER2xCD3 antibodies, activatable HER2xCD3 antibodies, CD20xCD3 antibodies, activatable CD20xCD3 antibodies, TROP2xCD3 antibodies, or activatable TROP2xCD3 antibodies) described herein.

In some embodiments, wherein the BiTE or the activatable BiTE target is HER2, the cancer is HER2-positive cancer. In some embodiments, the cancer is breast cancer. In some embodiments, the cancer is ovarian cancer. In some embodiments, the cancer is lung cancer.

In some embodiments, wherein the BiTE or the activatable BiTE target is CD20, the cancer is CD20-positive cancer. In some embodiments, the cancer is lymphoma. In some embodiments, the cancer is leukemia.

In some embodiments, wherein the BiTE or the activatable BiTE target is TROP2, the cancer is TROP2-positive cancer. In some embodiments, the cancer is breast cancer. In some embodiments, the cancer is lymphoma. In some embodiments, the cancer is selected from the group consisting of oral cavity SCC, carcinoma, non-small-cell lung, refractory, colorectal cancer, gastric adenocarcinoma, esophageal cancer, hepatocellular carcinoma, non-small-cell lung cancer, small-cell lung cancer, ovarian epithelial cancer, carcinoma breast stage IV, hormone-refractory prostate cancer, pancreatic ductal adenocarcinoma, head and neck cancers, squamous cell renal cell cancer, urinary bladder neoplasms, cervical cancer, endometrial cancer, follicular thyroid cancer, glioblastoma multiforme, and triple-negative breast cancer.

In some embodiments, wherein the BiTE or the activatable BiTE target is BCMA, the cancer is BCMA-positive cancer. In some embodiments, the cancer is lymphoma. In some embodiments, the cancer is leukemia.

In some embodiments, wherein the BiTE or the activatable BiTE target is CD19, the cancer is CD19-positive cancer. In some embodiments, the cancer is lymphoma. In some embodiments, the cancer is leukemia.

In some embodiments, there is provided a method for treating a cancer in a subject in need thereof comprising administering to the subject an effective amount of a pharmaceutical composition comprising an activatable multispecific antibody of the present disclosure (e.g., an activatable multispecific antibody that binds human CD3 and a target antigen such as HER2, CD20, TROP2, BCMA, or CD19). In some embodiments, the first cleavable moiety and the second cleavable moiety are cleaved at a diseased site, thereby unblocking binding of the multispecific activatable antibody to human CD3 and the target antigen at the diseased site.

In some embodiments, there is provided a method of enhancing an immune response in a mammal, which comprises administering to the mammal an effective amount of a pharmaceutical composition comprising any one of the multispecific antibodies, activatable antibodies, isolated anti-CD3 antibodies or antigen-binding fragments thereof, or activatable multispecific antibodies described herein. The term “enhancing immune response” or its grammatical variations, means stimulating, evoking, increasing, improving, or augmenting any response of a subject's immune system. The immune response may be a cellular response (i.e. cell-mediated, such as cytotoxic T lymphocyte mediated) or a humoral response (i.e. antibody-mediated response), and may be a primary or secondary immune response. Examples of enhancement of immune response include activation of PBMCs and/or T cells (including increasing secretion of one or more cytokines such as IL-2 and/or IFNγ). The enhancement of immune response can be assessed using a number of in vitro or in vivo measurements known to those skilled in the art, including, but not limited to, cytotoxic T lymphocyte assays, release of cytokines, regression of tumors, survival of tumor bearing animals, antibody production, immune cell proliferation, expression of cell surface markers, and cytotoxicity. Typically, methods of the present application enhance the immune response by a mammal when compared to the immune response by an untreated mammal or a mammal not treated using the recited methods.

Also provided herein is a method of reducing the level of cytokine release and/or the severity of one or more side effects, wherein the method comprises administering to the mammal an effective amount of a pharmaceutical composition comprising any one of the multispecific antibodies, activatable antibodies, isolated anti-CD3 antibodies or antigen-binding fragments thereof, or activatable multispecific antibodies described herein. In some embodiments, the level of cytokine release and/or the severity of one or more side effects are reduced relative to the level of cytokine release and/or the severity of one or more side effects in response to administration of a reference antibody (e.g., an activatable BiTE molecule with a stronger CD3 binding affinity, an activatable BiTE molecule with a weaker masking efficiency, or a masked BiTE molecule having no cleavable moieties). In some embodiments, the level of cytokine release is 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, or 500-fold reduced relative to the level of cytokine release in response to administration of the reference antibody. Methods of measuring cytokine release are described herein and known in the art (see, e.g., the methods of Example 7). In some embodiments, the level of the cytokines IFNγ, IL-2, IL-6, TNFα, IL-5, and/or IL-4 released is measured. In some embodiments, the side effect is selected from the group consisting of fever, inflammation, severe fatigue, and nausea. In some embodiments, the side effect is a side effect associated with BiTE administration. In some embodiments, hypercytokinemia is prevented. In some embodiments, a cytokine storm is prevented. In some embodiments, cytokine release is prevented.

In practicing the therapeutic methods, the multispecific antibodies, activatable antibodies, isolated anti-CD3 antibodies or antigen-binding fragments thereof, and activatable multispecific antibodies described herein may be administered alone as monotherapy, or administered in combination with one or more additional therapeutic agents or therapies. Thus, in another aspect, the present application provides a combination therapy, which comprises a multispecific antibodies, activatable antibody, isolated anti-CD3 antibody or antigen-binding fragment thereof, or activatable multispecific antibody described herein in combination with one or more additional therapies or therapeutic agents for separate, sequential or simultaneous administration. The term “additional therapeutic agent” may refer to any therapeutic agent other than an activatable antibody, isolated anti-CD3 antibody or antigen-binding fragment thereof, or activatable multispecific antibody provided by the application.

A wide variety of cancer therapeutic agents may be used in combination with an activatable antibody, isolated anti-CD3 antibody or antigen-binding fragment thereof, or activatable multispecific antibody provided by the present application. One of ordinary skill in the art will recognize the presence and development of other cancer therapies, which can be used in combination with the methods and multispecific antibodies, activatable antibodies, isolated anti-CD3 antibodies or antigen-binding fragments thereof, or activatable multispecific antibodies of the present application, and will not be restricted to those forms of therapy set forth herein. Examples of categories of additional therapeutic agents that may be used in the combination therapy for treating cancer include (1) chemotherapeutic agents, (2) immunotherapeutic agents, and (3) hormone therapeutic agents. In some embodiments, the additional therapeutic is a viral gene therapy, an immune checkpoint inhibitor, a target therapy, a radiation therapies, and/or a chemotherapeutic. In some embodiments, the combination therapy comprises surgery to remove a tumor.

In some embodiments, an activatable antibody, isolated anti-CD3 antibody or antigen-binding fragment thereof, or activatable multispecific antibody provided by the present application is administered in combination with an anti-PD-1 or anti-PD-L1 antibody. In some embodiments, the anti-PD-1 antibody comprises a heavy chain variable domain comprising the amino acid sequence of QVQLVQSGAEVKKPGSSVKVSCKASGFTFTTYYISWVRQAPGQGLEYLGYINMGSGGTNYNEKFKGRVTITADK STSTAYMELSSLRSEDTAVYYCAIIGYFDYWGQGTMVTVSS (SEQ ID NO:708) and/or a light chain variable domain comprising the amino acid sequence of DVVMTQSPLSLPVTLGQPASISCRSSQSLLDSDGGTYLYWFQQRPGQSPRRLIYLVSTLGSGVPDRFSGSGSGT DFTLKISRVEAEDVGVYYCMQLTHWPYTFGQGTKLEIKR (SEQ ID NO:709). In some embodiments, the anti-PD-1 antibody is toripalimab or pembrolizumab. In some embodiments, the anti-PD-L1 antibody is atezolizumab.

In some embodiments, an activatable antibody, isolated anti-CD3 antibody or antigen-binding fragment thereof, or activatable multispecific antibody provided by the present application is administered in combination with an anti-CD137 agonist or antibody. In some embodiments, the anti-CD137 agonist or antibody comprises a heavy chain variable region and a light chain variable region, wherein the heavy chain variable region comprises a CDR-H1 comprising the amino acid sequence of TGGVGVG (SEQ ID NO:700), a CDR-H2 comprising the amino acid sequence of LIDWADDKYYSPSLKS (SEQ ID NO:701), and a CDR-H3 comprising the amino acid sequence of GGSDTVIGDWFAY (SEQ ID NO:702); and/or wherein the light chain variable region comprises a CDR-L1 comprising the amino acid sequence of RASQSIGSYLA (SEQ ID NO:703), a CDR-L2 comprising the amino acid sequence of DASNLET (SEQ ID NO:704), and a CDR-L3 comprising the amino acid sequence of QQGYYLWT (SEQ ID NO:705). In some embodiments, the heavy chain variable region comprises the amino acid sequence of EVQLVESGGGLVQPGGSLRLSCAASGFSLSTGGVGVGWIRQAPGKGLEWLALIDWADD KYYSPSLKSRLTISRDNSKNTLYLQLNSLRAEDTAVYYCARGGSDTVIGDWFAYWGQG TLVTVSS (SEQ ID NO:706), and/or the light chain variable region comprises the amino acid sequence of DIQLTQSPSSLSASVGDRVTITCRASQSIGSYLAWYQQKPGKAPKLLIYDASNLETGVPSR FSGSGSGTDFTLTISSLQPEDFATYYCQQGYYLWTFGQGTKVEIKR (SEQ ID NO:707). In some embodiments, the heavy chain comprises the amino acid sequence of EVQLVESGGGLVQPGGSLRLSCAASGFSLSTGGVGVGWIRQAPGKGLEWLALIDWADDKYYSPS LKSRLTISRDNSKNTLYLQLNSLRAEDTAVYYCARGGSDTVIGDWFAYWGQGTLVTVSSASTKG PSVFPLAPCSRSTSESTAALGCLVKDYFPEPVTVSWNSGALTSGVHTFPAVLQSSGLYSLSSVV TVPSSSLGTKTYTCNVDHKPSNTKVDKRVESKYGPPCPPCPAPEFLGGPSVFLFPPKPKDTLMI SRTPEVTCVVVDVSQEDPEVQFNWYVDGVEVHNAKTKPREEQFNSTYRVVSVLTVLHQDWLNGK EYKCKVSNKGLPSSIEKTISKAKGQPREPQVYTLPPSQEEMTKNQVSLTCLVKGFYPSDIAVEW ESNGQPENNYKTTPPVLDSDGSFFLYSRLTVDKSRWQEGNVFSCSVMHEALHNHYTQKSLSLSL G (SEQ ID NO:710), and/or the light chain comprises the amino acid sequence of DIQLTQSPSSLSASVGDRVTITCRASQSIGSYLAWYQQKPGKAPKLLIYDASNLETGVPSRFSG SGSGTDFTLTISSLQPEDFATYYCQQGYYLWTFGQGTKVEIKRTVAAPSVFIFPPSDEQLKSGT ASVVCLLNNFYPREAKVQWKVDNALQSGNSQESVTEQDSKDSTYSLSSTLTLSKADYEKHKVYA CEVTHQGLSSPVTKSFNRGEC (SEQ ID NO:711). In some embodiments, the anti-CD137 agonist or antibody is an anti-CD137 antibody described WO2019036855.

The dosage, dosing frequency, route of administration for the therapeutic methods described herein depend on a number of factors, such as the type, and severity of the disorder to be treated, the particular activatable antibody, isolated anti-CD3 antibody or antigen-binding fragment thereof, or activatable multispecific antibody administered, the time of administration, the duration of the treatment, the particular additional therapy administered, the age, sex, weight, condition, general health and prior medical history of the patient being treated, and like factors known in the medical arts.

In some embodiments, a pharmaceutical composition, activatable antibody, isolated anti-CD3 antibody or antigen-binding fragment thereof, or activatable multispecific antibody provided by the present application is administered at a dose of 0.02 mg/kg, 0.2 mg/kg, 2 mg/kg, 10 mg/kg, 30 mg/kg, or 60 mg/kg. In some embodiments, the multispecific antibody, isolated antibody or antigen-binding fragment thereof, or masked antibody comprises: a first polypeptide comprising an amino acid sequence having at least 90% sequence identity with SEQ ID NO: 427, a second polypeptide comprising an amino acid sequence having at least 90% sequence identity with SEQ ID NO: 428, and a third polypeptide comprising an amino acid sequence having at least 90% sequence identity with SEQ ID NO: 112; a first polypeptide comprising an amino acid sequence having at least 90% sequence identity with SEQ ID NO: 83, a second polypeptide comprising an amino acid sequence having at least 90% sequence identity with SEQ ID NO: 84, and a third polypeptide comprising an amino acid sequence having at least 90% sequence identity with SEQ ID NO: 85; a first polypeptide comprising an amino acid sequence having at least 90% sequence identity with SEQ ID NO: 683, a second polypeptide comprising an amino acid sequence having at least 90% sequence identity with SEQ ID NO: 684, and a third polypeptide comprising an amino acid sequence having at least 90% sequence identity with SEQ ID NO: 685; a first polypeptide comprising the amino acid sequence of SEQ ID NO: 427, a second polypeptide comprising the amino acid sequence of SEQ ID NO: 428, and a third polypeptide comprising the amino acid sequence of SEQ ID NO: 112; a first polypeptide comprising the amino acid sequence of SEQ ID NO: 83, a second polypeptide comprising the amino acid sequence of SEQ ID NO: 84, and a third polypeptide comprising the amino acid sequence of SEQ ID NO: 85; a first polypeptide comprising the amino acid sequence of SEQ ID NO: 683, a second polypeptide comprising the amino acid sequence of SEQ ID NO: 684, and a third polypeptide comprising the amino acid sequence of SEQ ID NO: 685; a first polypeptide comprising the amino acid sequence of SEQ ID NO: 427 without the C-terminal lysine, a second polypeptide comprising the amino acid sequence of SEQ ID NO: 428 without the C-terminal lysine, and a third polypeptide comprising the amino acid sequence of SEQ ID NO: 112; a first polypeptide comprising the amino acid sequence of SEQ ID NO: 83, a second polypeptide comprising the amino acid sequence of SEQ ID NO: 84 without the C-terminal lysine, and a third polypeptide comprising the amino acid sequence of SEQ ID NO: 85 without the C-terminal lysine; or a first polypeptide comprising the amino acid sequence of SEQ ID NO: 683, a second polypeptide comprising the amino acid sequence of SEQ ID NO: 684 without the C-terminal lysine, and a third polypeptide comprising the amino acid sequence of SEQ ID NO: 685 without the C-terminal lysine.

Cancer treatments can be evaluated by, e.g., tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### EXAMPLES

The examples below are intended to be purely exemplary of the invention and should therefore not be considered to limit the invention in any way. The following examples and detailed description are offered by way of illustration and not by way of limitation.

### Example 1. Biophysical Characterization of Heterodimeric HER2xCD3 T-Cell-Engaging Bispecific Antibody

A heterodimeric bispecific scaffold was designed using the TYM13 Fc mutant (D or E356K:E357K:S364K:S400C-L351′D:K370′D:N390′C:K439′D; see CH3 SEQ ID NOs: 1-2). A light chain-heavy chain half antibody and a scFv-Fc chain were combined to form a bispecific antibody, with TYM13 mutations in hetero-Fc domain (see FIG. 4). A HER2xCD3 bispecific T-cell-engaging antibody (TY24051) was constructed using this scaffold. For comparison, corresponding antibodies having knobs-into-holes mutations Y394C, T366S, L368A, Y407V-S354′C T366′W and “Xencor mutations” E357Q, S364K-L368′D, K370'S were also constructed.

Plasmids encoding the heavy chain, light chain, and scFv-Fc chain of bispecific antibodies were transiently transfected into mammalian cells. Bispecific antibody-containing cell culture supernatants were harvested 7 days after transfection by centrifugation at 14000 g for 30 minutes and were filtered through a sterile filter (0.22 μm). Antibodies were purified by protein A affinity chromatography using MabSelect SuRe prepacked columns (GE Healthcare) and were subsequently buffer exchanged in 20 mM histidine (pH 5.5) buffer.

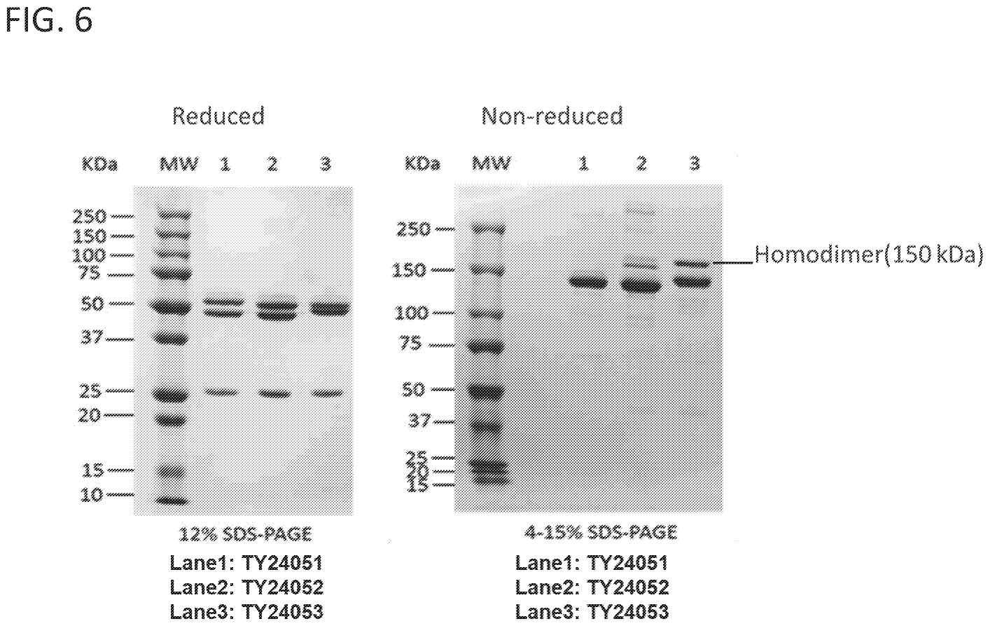

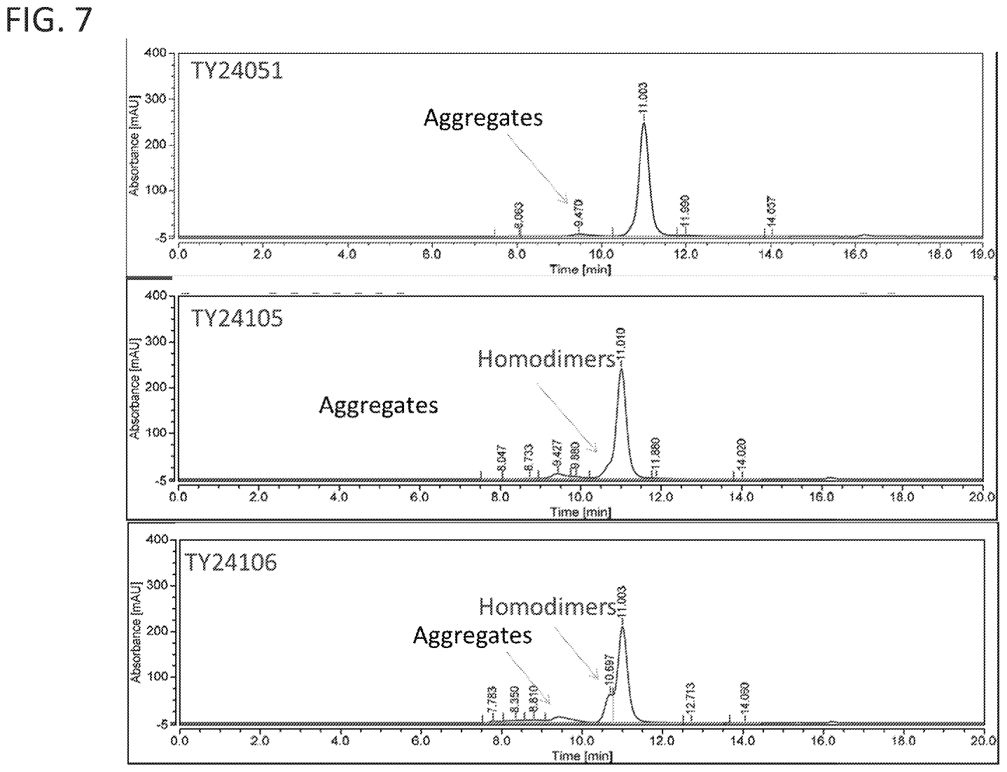

The biophysical purity of heterodimeric bispecific antibodies was assessed through SEC-HPLC and SDS-PAGE. As shown in FIG. 6 and FIG. 7, TY24051 with TYM13 mutations showed very good heterodimeric purity, with no detectable homodimers, while TY24105 and TY24106, with knobs-into-holes and Xencor mutation, both contained some 150 kDa homodimers (indicated in SDS-PAGE and SEC-HPLC graphs in FIG. 6 and FIG. 7, respectively). Furthermore, TY24051 contained fewer aggregates than TY24105 and TY24106.

When TY24051 was converted into an activatable antibody, TY24052, some aggregates were generated (see Table 1). TY24052 could be purified by cation exchange chromatography (CEX).

TABLE 1
Bispecific antibodies and their SEC-HPLC purity

| | | Antibody | SEC-HPLC purity | | |
|---|---|---|---|---|---|
| | | chain SEQ | HMW | Monomer | LMW |
| IgG ID | Fc mutant | ID NOs. | (%) | (%) | (%) |
| TY24051 | TYM13 | 112, | 3.1 | 96.0 | 0.9 |
| | | 113, 114 | | | |
| TY24105 | Knobs-into-holes | — | 9.2 | 89.2 | 1.5 |
| TY24106 | Xencor mutation | — | 31.6 | 68.2 | 0.2 |

### Example 2. Activatable Bispecific Antibody Construction and Functional Characterization

An activatable HER2xCD3 bispecific antibody (also referred herein as “SAFEbody” or “SAFE-bispecific”) was constructed (FIG. 5A). The constructs are described in Tables 2 and 3A.

TABLE 2
Bispecific antibodies and their purity determined by SEC-HPLC

| | | Antibody | SEC-HPLC purity | | |
|---|---|---|---|---|---|
| | | chain SEQ | HMW | Monomer | LMW |
| IgG ID | Format | ID NOs. | (%) | (%) | (%) |
| TY24051 | TYM13 N297A | 112, | 3.1 | 96.0 | 0.9 |
| | | 113, 114 | | | |
| TY24052 | TYM13 N297A, | 115, | 9.3 | 88.2 | 2.5 |
| | SAFE-bispecific | 116, 117 | | | |

TABLE 3A
Design of HER2 × CD3 bispecific SAFEbody

| | | | | | Fc | |
|---|---|---|---|---|---|---|
| | SAFEbody | Left | Right | Cleavable | heterodimeric | Fc |
| IgG ID | Motifs | Arm | Arm | activation | motif | effector |
| TY24051 | Parental bispecific | Fab | scFv | NA | TYM13 | N297A |
| | Fab X scFv in | (HER2) | (CD3) | | | |
| | IgG1 | | | | | |
| TY24052 | SAFE Bispecific | SAFE | SAFE | Cleavable | TYM13 | N297A |
| | Fab X scFv in | Fab | scFv | | | |
| | IgG1 | (HER2) | (CD3) | | | |
| TY24053 | SAFE Bispecific | SAFE | SAFE | Non- | TYM13 | N297A |
| | Fab X scFv in | Fab | scFv | cleavable | | |
| | IgG1 | (HER2) | (CD3) | | | |
| TY24110 | SAFE Bispecific | SAFE | SAFE | Cleavable | TYM13 | N297A |
| | Fab X scFv in | Fab | scFv | | | |
| | IgG1 | (HER2) | (CD3) | | | |
| TY24111 | SAFE Bispecific | Fab | SAFE | Cleavable | TYM13 | N297A |
| | Fab X scFv in | (HER2) | scFv | | | |
| | IgG1 | | (CD3) | | | |

### A. Enzyme-Linked Immunosorbent Assay (ELISA)

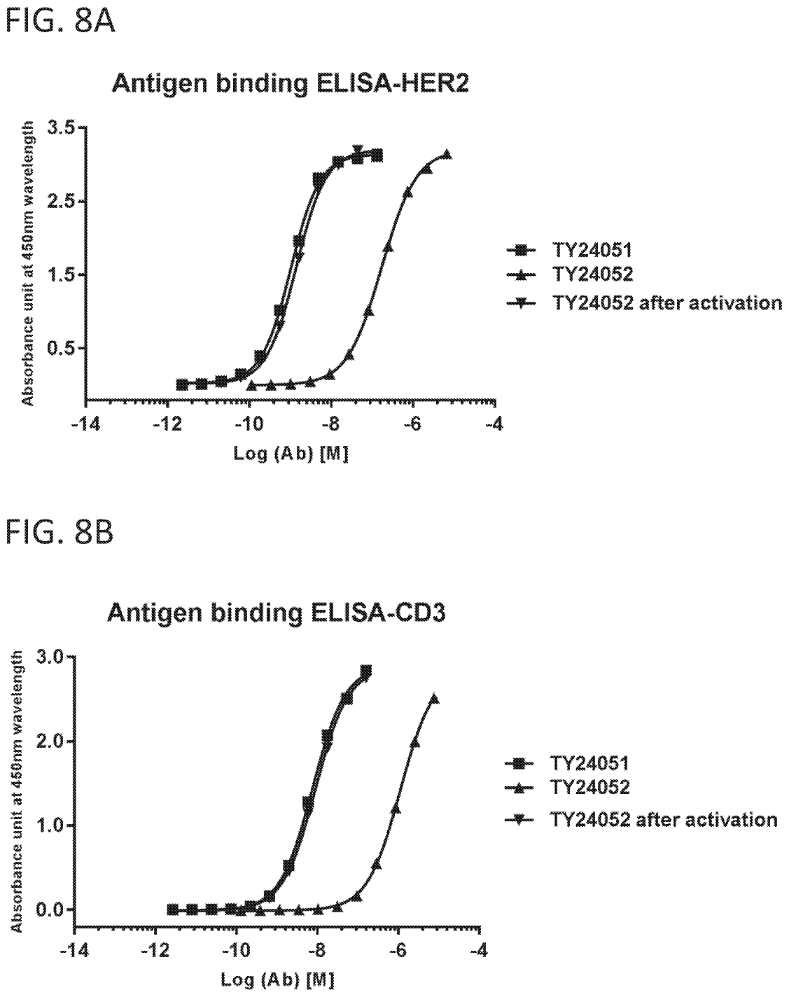

The affinities of the bispecific antibody (TY24051) and its SAFEbody version (TY24052) were analyzed through enzyme-linked immunosorbent assay (ELISA) assay. 2 μg/mL of human HER2 or CD3 (ε and δ chain heterodimer) fused with human Fc fragment, were prepared and used to coat the ELISA plate at 2-8° C. overnight. After washing and blocking, 50 μL serial diluted IgGs were added and incubated at 37° C. for 1 hour. Plates were washed three times and then incubated with 50 μL/well TMB substrate at room temperature for about 20 minutes. Absorbance at 450 nm was measured after the reaction was stopped. The data was analyzed by GraphPad Prism 6 with nonlinear fitting. As shown in FIGS. 8A-8B, TY24051 bound to both HER2 and CD3, while TY24052 showed an apparently lower affinity than TY24051. After activation, the affinity of TY24052 was fully recovered.

### B. Tumor Killing Assay

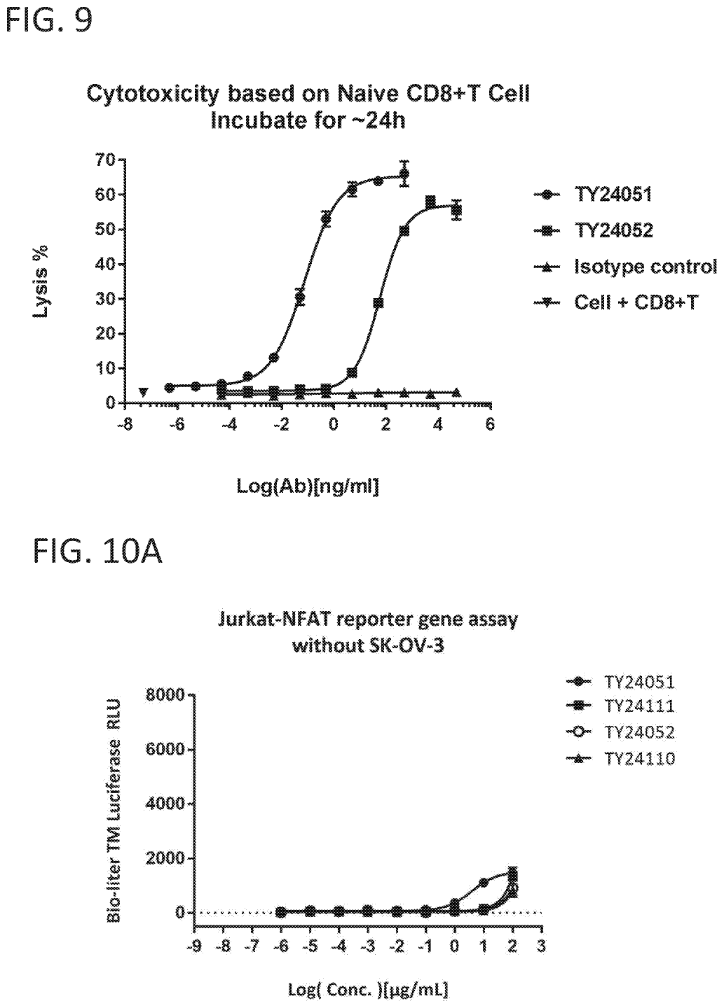

To compare functional activity between TY24051 and TY24052, the antibodies were expressed, purified and evaluated for antigen-dependent bispecific antibody-mediated tumor cell killing activity (FIG. 9). For in vitro cytotoxicity assays, naïve human pan-T-cells were isolated from fresh human blood and mixed with HER2-positive tumor cells (SK-OV-3) along with increasing amounts of bispecific antibody for 24 hours (target cells:1×10⁴ cells/well, E:T=10:1). As show in FIG. 9, dose dependent killing was observed for TY24051 and TY24052, and TY24052 showed about 800-fold increase the EC₅₀, compared with TY24051. No specific killing was observed with isotype control.

### C. Jurkat NFAT Reporter Assay to Measure T-Cell Activation

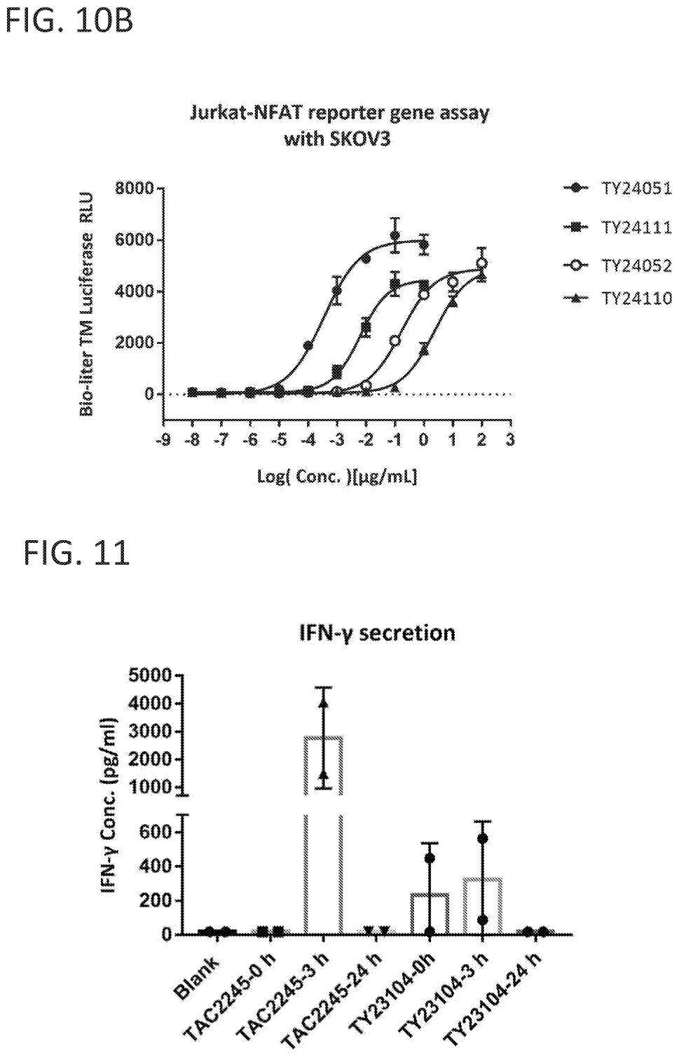

The activities of TY24051, TY24052, TY24110 and TY24111 were tested in a Jurkat NFAT reporter assay in the presence or absence of target SK-OV-3 cells (FIGS. 10A and 10B, respectively). Jurkat-NFAT cells are immortalized T cells engineered to driven by an NFAT response element. Jurkat-NFAT cells express luciferase upon T-cell activation.

Jurkat-NFAT cells and SK-OV-3 cells were thawed, washed and then cultured in the incubator for the reporter potency assay. The effector cells (Jurkat-NFAT cells) were mixed with target cells (SK-OV-3 cells) in a final cell ratio of 5:1 Jurkat-NFAT cells: SK-OV-3 cells. Sample dilutions of bispecific antibodies were prepared in assay medium consisting of RPM 1640 supplemented with 1% FBS. Plates were placed in a 37° C. incubator supplemented with 5% CO2 for approximately 6 hours. After incubation, 70 μL/well Bio-lite™ Luciferase Assay buffer was added to each well and 100 μL supernatant was collected for measuring luminescence using a plate reader.

As shown in FIG. 10A, no response was detected in absence of target cells. In contrast, FIG. 10B shows that parental bispecific antibody TY24051, the single-arm masked activatable bispecific antibody TY24111, and the two-arm masked activatable bispecific antibodies TY24052 and TY24110 induced a dose-dependent increase in luciferase in the presence of the target cells. The parental bispecific antibody TY24051 shows the strongest potency in that it activated the reporter at the lowest concentration of antibody. The single-arm masked bispecific antibody TY24111 was less potent than TY24051 and more potent than the activatable bispecific antibodies TY24052 and TY24110. Therefore, activatable bispecific antibodies with variable potencies were generated.

### Example 3. In Vivo Characterization of Activatable Anti-CD3 Antibodies

The following example describes the generation of activatable anti-CD3 antibodies with various masking moieties, and in vivo effects of administering anti-CD3 antibodies.

### A. Generation of Activatable Anti-CD3 Antibodies

Parental antibody TAC2245 was used as the basis for generating activatable anti-CD3 antibodies. TAC2245, which is also known as huOKT3-C114S-gLC (see U.S. Publication No. US20140170149) has one amino acid substitution relative to the anti-CD3 antibody Teplizumab. Specifically, the cysteine residue at position 114 in the heavy chain variable region of Teplizumab was substituted to a serine residue (C114S) to create TAC2245. The sequences of the TAC2245 heavy chain variable region (VH) and light chain variable region (VL) are provided below, with the CDR sequences bolded, underlined, and italicized, and the CDRs are also shown in Table 3B. In the VH, the serine residue at position 114 is shown in lower case.

>TAC2245_VH
(SEQ ID NO: 366)
QVQLVQSGGGVVQPGRSLRLSCKASGYTFTRYTMHWVRQAPGKGLEWIG
YINPSRGYTNYNQKVKDRFTISRDNSKNTAFLQMDSLRPEDTGVYFCAR
YYDDHYSLDYWGQGTPVTVSS
>TAC2245_VL
(SEQ ID NO: 367)
DIQMTQSPSSLSASVGDRVTITCSASSSVSYMNWYQQTPGKAPKRWIYD
TSKLASGVPSRFSGSGSGTDYTFTISSLQPEDIATYYCQQWSSNPFTFG
QGTKLQIT

TABLE 3B
Amino acid sequences of TAC2245 CDRs

| CDR | TAC2245 | SEQ ID NO |
|---|---|---|
| CDR-H1 | RYTMH | 368 |
| CDR-H2 | YINPSRGYTNYNQKVKD | 369 |
| CDR-H3 | YYDDHYSLDY | 370 |
| CDR-L1 | SASSSVSYMN | 371 |
| CDR-L2 | DTSKLAS | 372 |
| CDR-L3 | QQWSSNPFT | 373 |

Activatable anti-CD3 antibodies TY23100, TY23101, TY23102, and TY23104 were generated from parental antibody TAC2245 by the addition of a masking moiety and cleavage moiety to the N-terminus of the light chain. The constructs are described in Table 3C, with the sequences of the masking moieties underlined and bolded.

TABLE 3C
Amino acid sequences of TY23100, TY23101, TY23102, and TY23104 heavy and
light chains

| | | | SEQ ID | SEQ ID |
|---|---|---|---|---|
| | | | NO of | of |
| IgG | Antibody | | antibody | masking |
| ID | chain | Amino Acid Sequence | chain | moiety |
| TY23100 | LC | EVGSYYDNYNDCDNYDDDCYYSGRSAGGGGTPLGLAGSGGS | 577 | 585 |
| | | DIQMTQSPSSLSASVGDRVTITCSASSSVSYMNWYQQTPGK | | |
| | | APKRWIYDTSKLASGVPSRFSGSGSGTDYTFTISSLQPEDI | | |
| | | ATYYCQQWSSNPFTFGQGTKLQITRTVAAPSVFIFPPSDEQ | | |
| | | LKSGTASVVCLLNNFYPREAKVQWKVDNALQSGNSQESVTE | | |
| | | QDSKDSTYSLSSTLTLSKADYEKHKVYACEVTHQGLSSPVT | | |
| | | KSFNRGEC | | |
| TY23100 | HC | QVQLVQSGGGVVQPGRSLRLSCKASGYTFTRYTMHWVRQAP | 578 | n/a |
| | | GKGLEWIGYINPSRGYTNYNQKVKDRFTISRDNSKNTAFLQ | | |
| | | MDSLRPEDTGVYFCARYYDDHYSLDYWGQGTPVTVSSASTK | | |
| | | GPSVFPLAPCSRSTSESTAALGCLVKDYFPEPVTVSWNSGA | | |
| | | LTSGVHTFPAVLQSSGLYSLSSVVTVPSSSLGTKTYTCNVD | | |
| | | HKPSNTKVDKRVESKYGPPCPPCPAPEFLGGPSVFLFPPKP | | |
| | | KDTLMISRTPEVTCVVVDVSQEDPEVQFNWYVDGVEVHNAK | | |
| | | TKPREEQFNSTYRVVSVLTVLHQDWLNGKEYKCKVSNKGLP | | |
| | | SSIEKTISKAKGQPREPQVYTLPPSQEEMTKNQVSLTCLVK | | |
| | | GFYPSDIAVEWESNGQPENNYKTTPPVLDSDGSFFLYSRLT | | |
| | | VDKSRWQEGNVFSCSVMHEALHNHYTQKSLSLSLGK | | |
| TY23101 | LC | EVGSYNADYHQCSDVPTDCLDSGRSAGGGGTPLGLAGSGGS | 579 | 586 |
| | | DIQMTQSPSSLSASVGDRVTITCSASSSVSYMNWYQQTPGK | | |
| | | APKRWIYDTSKLASGVPSRFSGSGSGTDYTFTISSLQPEDI | | |
| | | ATYYCQQWSSNPFTFGQGTKLQITRTVAAPSVEIFPPSDEQ | | |
| | | LKSGTASVVCLLNNFYPREAKVQWKVDNALQSGNSQESVTE | | |
| | | QDSKDSTYSLSSTLTLSKADYEKHKVYACEVTHQGLSSPVT | | |
| | | KSFNRGEC | | |
| TY23101 | HC | QVQLVQSGGGVVQPGRSLRLSCKASGYTFTRYTMHWVRQAP | 580 | n/a |
| | | GKGLEWIGYINPSRGYTNYNQKVKDRFTISRDNSKNTAFLQ | | |
| | | MDSLRPEDTGVYFCARYYDDHYSLDYWGQGTPVTVSSASTK | | |
| | | GPSVFPLAPCSRSTSESTAALGCLVKDYFPEPVTVSWNSGA | | |
| | | LTSGVHTFPAVLQSSGLYSLSSVVTVPSSSLGTKTYTCNVD | | |
| | | HKPSNTKVDKRVESKYGPPCPPCPAPEFLGGPSVFLFPPKP | | |
| | | KDTLMISRTPEVTCVVVDVSQEDPEVQFNWYVDGVEVHNAK | | |
| | | TKPREEQFNSTYRVVSVLTVLHQDWLNGKEYKCKVSNKGLP | | |
| | | SSIEKTISKAKGQPREPQVYTLPPSQEEMTKNQVSLTCLVK | | |
| | | GFYPSDIAVEWESNGQPENNYKTTPPVLDSDGSFFLYSRLT | | |
| | | VDKSRWQEGNVFSCSVMHEALHNHYTQKSLSLSLGK | | |
| TY23102 | LC | EVGSYDSYDYNCYHDHHTCHDSGRSAGGGGTPLGLAGSGGS | 581 | 587 |
| | | DIQMTQSPSSLSASVGDRVTITCSASSSVSYMNWYQQTPGK | | |
| | | APKRWIYDTSKLASGVPSRFSGSGSGTDYTFTISSLQPEDI | | |
| | | ATYYCQQWSSNPFTFGQGTKLQITRTVAAPSVEIFPPSDEQ | | |
| | | LKSGTASVVCLLNNFYPREAKVQWKVDNALQSGNSQESVTE | | |
| | | QDSKDSTYSLSSTLTLSKADYEKHKVYACEVTHQGLSSPVT | | |
| | | KSFNRGEC | | |
| TY23102 | HC | QVQLVQSGGGVVQPGRSLRLSCKASGYTFTRYTMHWVRQAP | 582 | n/a |
| | | GKGLEWIGYINPSRGYTNYNQKVKDRFTISRDNSKNTAFLQ | | |
| | | MDSLRPEDIGVYFCARYYDDHYSLDYWGQGTPVTVSSASTK | | |
| | | GPSVFPLAPCSRSTSESTAALGCLVKDYFPEPVTVSWNSGA | | |
| | | LTSGVHTFPAVLQSSGLYSLSSVVTVPSSSLGTKTYTCNVD | | |
| | | HKPSNTKVDKRVESKYGPPCPPCPAPEFLGGPSVFLFPPKP | | |
| | | KDTLMISRTPEVTCVVVDVSQEDPEVQFNWYVDGVEVHNAK | | |
| | | TKPREEQFNSTYRVVSVLTVLHQDWLNGKEYKCKVSNKGLP | | |
| | | SSIEKTISKAKGQPREPQVYTLPPSQEEMTKNQVSLTCLVK | | |
| | | GFYPSDIAVEWESNGQPENNYKTTPPVLDSDGSFFLYSRLT | | |
| | | VDKSRWQEGNVFSCSVMHEALHNHYTQKSLSLSLGK | | |
| TY23104 | LC | EVGSYAYHDDDCPDDDYDCASSGRSAGGGGTPLGLAGSGGS | 583 | 588 |
| | | DIQMTQSPSSLSASVGDRVTITCSASSSVSYMNWYQQTPGK | | |
| | | APKRWIYDTSKLASGVPSRFSGSGSGTDYTFTISSLQPEDI | | |
| | | ATYYCQQWSSNPFTFGQGTKLQITRTVAAPSVFIFPPSDEQ | | |
| | | LKSGTASVVCLLNNFYPREAKVQWKVDNALQSGNSQESVTE | | |
| | | QDSKDSTYSLSSTLTLSKADYEKHKVYACEVTHQGLSSPVT | | |
| | | KSFNRGEC | | |
| TY23104 | HC | QVQLVQSGGGVVQPGRSLRLSCKASGYTFTRYTMHWVRQAP | 584 | n/a |
| | | GKGLEWIGYINPSRGYTNYNQKVKDRFTISRDNSKNTAFLQ | | |
| | | MDSLRPEDTGVYFCARYYDDHYSLDYWGQGTPVTVSSASTK | | |
| | | GPSVFPLAPCSRSTSESTAALGCLVKDYFPEPVTVSWNSGA | | |
| | | LTSGVHTFPAVLQSSGLYSLSSVVTVPSSSLGTKTYTCNVD | | |
| | | HKPSNTKVDKRVESKYGPPCPPCPAPEFLGGPSVFLFPPKP | | |
| | | KDTLMISRTPEVTCVVVDVSQEDPEVQFNWYVDGVEVHNAK | | |
| | | TKPREEQFNSTYRVVSVLTVLHQDWLNGKEYKCKVSNKGLP | | |
| | | SSIEKTISKAKGQPREPQVYTLPPSQEEMTKNQVSLTCLVK | | |
| | | GFYPSDIAVEWESNGQPENNYKTTPPVLDSDGSFFLYSRLT | | |
| | | VDKSRWQEGNVFSCSVMHEALHNHYTQKSLSLSLGK | | |

In addition, parental antibody TAC2225 was used as the basis for generating further activatable anti-CD3 antibodies. The VL of TAC2225 is set forth in SEQ ID NO: 68, and the VH is set forth in SEQ ID NO: 67 (see Table C). Activatable anti-CD3 antibodies were generated from parental antibody TAC2225 by the addition of a masking moiety and cleavage moiety to the N-terminus of the light chain. The constructs are described in Table 3D), with the sequences of the masking moieties underlined and bolded.

TABLE 3D
Amino acid sequences of TY23105, TY23110, TY23115, and TY23118 heavy and
light chains

| | | | SEQ ID | SEQ ID |
|---|---|---|---|---|
| | | | NO of | NO of |
| | Antibody | | antibody | masking |
| IgG ID | chain | Amino Acid Sequence | chain | moiety |
| TY23105 | LC | EVGSYDDPDFPCDPDDADCPNSGRSAGGGGTPLGLAGSG | 589 | 597 |
| | | GSQAVVTQEPSLTVSPGGTVTLTCGSSTGAVTTSNYANW | | |
| | | VQQKPGQAPRGLIGGTNKRAPGVPARFSGSLLGGKAALT | | |
| | | LSGAQPEDEAEYYCALWYSNLWVFGGGTKLTVLRTVAAP | | |
| | | SVFIFPPSDEQLKSGTASVVCLLNNFYPREAKVQWKVDN | | |
| | | ALQSGNSQESVTEQDSKDSTYSLSSTLTLSKADYEKHKV | | |
| | | YACEVTHQGLSSPVTKSFNRGEC | | |
| TY23105 | HC | EVQLVESGGGLVQPGGSLRLSCAASGFTFNTYAMNWVRQ | 590 | |
| | | APGKGLEWVGRIRSKYNNYATYYADSVKGRFTISRDDSK | | |
| | | NTLYLQMNSLRAEDTAVYYCVRHGNFGNSYVSWFAYWGQ | | |
| | | GTLVTVSSASTKGPSVFPLAPSSKSTSGGTAALGCLVKD | | |
| | | YFPEPVTVSWNSGALTSGVHTFPAVLQSSGLYSLSSVVT | | |
| | | VPSSSLGTQTYICNVNHKPSNTKVDKKVEPKSCDKTHTC | | |
| | | PPCPAPELLGGPSVFLFPPKPKDTLMISRTPEVTCVVVD | | |
| | | VSHEDPEVKFNWYVDGVEVHNAKTKPREEQYNSTYRVVS | | |
| | | VLTVLHQDWLNGKEYKCKVSNKALPAPIEKTISKAKGQP | | |
| | | REPQVYTLPPSRDELTKNQVSLTCLVKGFYPSDIAVEWE | | |
| | | SNGQPENNYKTTPPVLDSDGSFFLYSKLTVDKSRWQQGN | | |
| | | VFSCSVMHEALHNHYTQKSLSLSPGK | | |
| TY23110 | LC | EVGSYAPHDPDCPADPPSCYPSGRSAGGGGTPLGLAGSG | 591 | 598 |
| | | GSQAVVTQEPSLTVSPGGTVTLTCGSSTGAVTTSNYANW | | |
| | | VQQKPGQAPRGLIGGTNKRAPGVPARFSGSLLGGKAALT | | |
| | | LSGAQPEDEAEYYCALWYSNLWVFGGGTKLTVLRTVAAP | | |
| | | SVFIFPPSDEQLKSGTASVVCLLNNFYPREAKVQWKVDN | | |
| | | ALQSGNSQESVTEQDSKDSTYSLSSTLTLSKADYEKHKV | | |
| | | YACEVTHQGLSSPVTKSFNRGEC | | |
| TY23110 | HC | EVQLVESGGGLVQPGGSLRLSCAASGFTFNTYAMNWVRQ | 592 | |
| | | APGKGLEWVGRIRSKYNNYATYYADSVKGRFTISRDDSK | | |
| | | NTLYLQMNSLRAEDTAVYYCVRHGNFGNSYVSWFAYWGQ | | |
| | | GTLVTVSSASTKGPSVFPLAPSSKSTSGGTAALGCLVKD | | |
| | | YFPEPVTVSWNSGALTSGVHTFPAVLQSSGLYSLSSVVT | | |
| | | VPSSSLGTQTYICNVNHKPSNTKVDKKVEPKSCDKTHTC | | |
| | | PPCPAPELLGGPSVFLFPPKPKDTLMISRTPEVTCVVVD | | |
| | | VSHEDPEVKFNWYVDGVEVHNAKTKPREEQYNSTYRVVS | | |
| | | VLTVLHQDWLNGKEYKCKVSNKALPAPIEKTISKAKGQP | | |
| | | REPQVYTLPPSRDELTKNQVSLTCLVKGFYPSDIAVEWE | | |
| | | SNGQPENNYKTTPPVLDSDGSFFLYSKLTVDKSRWQQGN | | |
| | | VFSCSVMHEALHNHYTQKSLSLSPGK | | |
| TY23115 | LC | EVGSYPYDDPDCPSHDSDCDNSGRSAGGGGTPLGLAGSG | 593 | 35 |
| | | GSQAVVTQEPSLTVSPGGTVTLTCGSSTGAVTTSNYANW | | |
| | | VQQKPGQAPRGLIGGTNKRAPGVPARFSGSLLGGKAALT | | |
| | | LSGAQPEDEAEYYCALWYSNLWVFGGGTKLTVLRTVAAP | | |
| | | SVFIFPPSDEQLKSGTASVVCLLNNFYPREAKVQWKVDN | | |
| | | ALQSGNSQESVTEQDSKDSTYSLSSTLTLSKADYEKHKV | | |
| | | YACEVTHQGLSSPVTKSFNRGEC | | |
| TY23115 | HC | EVQLVESGGGLVQPGGSLRLSCAASGFTENTYAMNWVRQ | 594 | |
| | | APGKGLEWVGRIRSKYNNYATYYADSVKGRFTISRDDSK | | |
| | | NTLYLQMNSLRAEDTAVYYCVRHGNFGNSYVSWFAYWGQ | | |
| | | GTLVTVSSASTKGPSVFPLAPSSKSTSGGTAALGCLVKD | | |
| | | YFPEPVTVSWNSGALTSGVHTFPAVLQSSGLYSLSSVVT | | |
| | | VPSSSLGTQTYICNVNHKPSNTKVDKKVEPKSCDKTHTC | | |
| | | PPCPAPELLGGPSVFLFPPKPKDTLMISRTPEVTCVVVD | | |
| | | VSHEDPEVKFNWYVDGVEVHNAKTKPREEQYNSTYRVVS | | |
| | | VLTVLHQDWLNGKEYKCKVSNKALPAPIEKTISKAKGQP | | |
| | | REPQVYTLPPSRDELTKNQVSLTCLVKGFYPSDIAVEWE | | |
| | | SNGQPENNYKTTPPVLDSDGSFFLYSKLTVDKSRWQQGN | | |
| | | VFSCSVMHEALHNHYTQKSLSLSPGK | | |
| TY23118 | LC | EVGSYDADDPDCPADNNHCHYSGRSAGGGGTPLGLAGSG | 595 | 599 |
| | | GSQAVVTQEPSLTVSPGGTVTLTCGSSTGAVTTSNYANW | | |
| | | VQQKPGQAPRGLIGGTNKRAPGVPARFSGSLLGGKAALT | | |
| | | LSGAQPEDEAEYYCALWYSNLWVFGGGTKLTVLRTVAAP | | |
| | | SVFIFPPSDEQLKSGTASVVCLLNNFYPREAKVQWKVDN | | |
| | | ALQSGNSQESVTEQDSKDSTYSLSSTLTLSKADYEKHKV | | |
| | | YACEVTHQGLSSPVTKSFNRGEC | | |
| TY23118 | HC | EVQLVESGGGLVQPGGSLRLSCAASGFTFNTYAMNWVRQ | 596 | |
| | | APGKGLEWVGRIRSKYNNYATYYADSVKGRFTISRDDSK | | |
| | | NTLYLQMNSLRAEDTAVYYCVRHGNFGNSYVSWFAYWGQ | | |
| | | GTLVTVSSASTKGPSVFPLAPSSKSTSGGTAALGCLVKD | | |
| | | YFPEPVTVSWNSGALTSGVHTFPAVLQSSGLYSLSSVVT | | |
| | | VPSSSLGTQTYICNVNHKPSNTKVDKKVEPKSCDKTHTC | | |
| | | PPCPAPELLGGPSVFLFPPKPKDTLMISRTPEVTCVVVD | | |
| | | VSHEDPEVKFNWYVDGVEVHNAKTKPREEQYNSTYRVVS | | |
| | | VLTVLHQDWLNGKEYKCKVSNKALPAPIEKTISKAKGQP | | |
| | | REPQVYTLPPSRDELTKNQVSLTCLVKGFYPSDIAVEWE | | |
| | | SNGQPENNYKTTPPVLDSDGSFFLYSKLTVDKSRWQQGN | | |
| | | VFSCSVMHEALHNHYTQKSLSLSPGK | | |

### B. Activatable Anti-Human CD3 Antibodies Did not Induce IFNγ Release in Humanized Peripheral Blood Mononuclear Cell Mouse Model

To generate a humanized peripheral blood mononuclear cell (PBMC) mouse model (huPBMC-NSG), fresh or frozen human PBMCs were isolated from a local healthy donor, and 10×10⁶ PBMCs were injected intravenously into NSG (NOD-scid IL2rγ^{null}) mice to reconstitute the immune system. Two weeks following PBMC injection, FACS was used to detect the reconstitution conditions in NSG mice. Mice with a greater than 15% reconstitution level were used for further study.

TAC2245 and an activatable huOKT3 anti-human CD3 antibody (TY23104, also known as an anti-CD3 SAFEbody), were administered in vivo to the huPBMC-NSG mice. 20 μg of each antibody was administered per mouse. The level of IFNγ was quantified over the course of 24 hours at 0, 3 and 24-hour time points, and IFNγ levels were measured in 3 mice per time point.

As shown in FIG. 11, IFNγ levels were significantly elevated within 3 hours and declined thereafter in mice treated with the parental anti-CD3 antibody TAC2245, which produced a strong induction of human cytokine IFNγ in both the fresh and frozen (data not shown) PBMC models. Mice treated with activatable anti-CD3 antibody TY23104 shows no significant increase in IFNγ levels. These results indicated that the activatable anti-CD3 antibody TY23104 had a good masking efficiency to prevent inducing acute cytokine release in huPBMC-NSG mice.

C. Activatable Cross-Reactive Anti-Human/Cynomolgus CD3 Antibodies Did not Induce IFNγ Release in huPBMC-NSG Mouse Model

huPBMC-NSG mice were generated as described above. Activatable antibodies TY23115 and TY23118 were derived from parental antibody TAC2225 as described above. TAC2225, TY23115 and TY23118 were administered in vivo to the huPBMC-NSG mice. 20 μg of each antibody was administered per mouse. The level of IFNγ was quantified over the course of 24 hours at 0, 3 and 24 hour time points, and IFNγ levels were measured in 3 mice per time point.

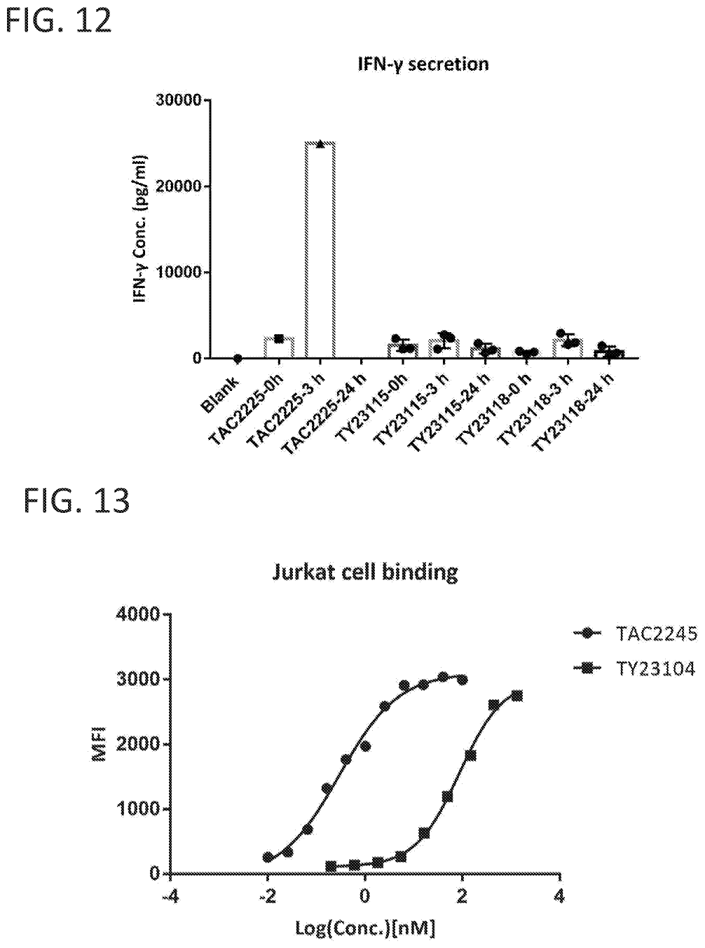

As shown in FIG. 12, mice treated with TAC2225 showed a strong induction of IFNγ. In the mice treated with TAC2225, IFNγ was significantly elevated within 3 hours and declined thereafter. Mice treated with the activatable anti-CD3 antibodies TY23115 or TY23118 showed no significant increase in IFNγ levels. Therefore, the activatable anti-CD3 antibodies TY23115 and TY23118 had good masking efficiencies that prevented the induction of acute cytokine release in huPBMC-NSG mice.

### D. Dose-Dependent Binding Assay of Parental and Activatable Anti-CD3 Antibodies to Jurkat Cells

The ability of the parental huOKT3 anti-CD3 antibody TAC2245 and the activatable huOKT3 anti-CD3 antibody TY23104 to bind Jurkat cells was measured in a dose-dependent binding assay.

The Jurkat cells were thawed, washed and cultured in an incubator. Diluted samples of antibody were prepared in assay medium consisting of RPM 1640 supplemented with 1% FBS as indicated in the figure. 4×10⁵ Jurkat cells/well were aliquoted into 96-well plate and incubated with parental huOKT3 TAC2245 or activatable huOKT3 for 1 hour at 4° C. After centrifugation and washing, Jurkat cells were further incubated with the secondary antibody anti-human IgG AF647 for 30 minutes at 4° C. After centrifugation and washing, the Jurkat cells labelled with antibodies were re-suspended in 2% FBS DPBS and analyzed by FACS.

As shown in FIG. 13, the parental anti-CD3 antibody bound the Jurkat cells at a lower concentration of antibody than the activatable antibody did. Therefore, the activatable anti-CD3 antibody TY23104 exhibited a good binding masking efficiency compared to parental anti-CD3 antibody TAC2245.

### E. Jurkat NFAT Reporter Assay of Parental and Activatable Anti-CD3 Antibodies

### Jurkat NFAT Reporter Assay of Human/Cynomolgus Cross-Reactive Anti-CD3 and Activatable Anti-CD3 Antibodies

The activities of parental (TAC2225) and activatable (TY23115 and TY23118) cross-reactive anti-CD3 antibodies were tested in a Jurkat NFAT reporter assay.

Jurkat-NFAT-luc2 cells were thawed, washed, and cultured in an incubator. Diluted samples of antibody were prepared in assay medium consisting of RPM 1640 supplemented with 1% FBS. 5×10⁴ Jurkat-NFAT-luc2 cells/well were added to the 96-well plate and incubated with 20 μL of cross-reactive anti-CD3 TAC2225 or the activatable antibodies TY23115, or TY23118 overnight. After incubation of about 17 hours, 100 μL/well ONE-Glo Luciferase Assay buffer was added to the 96-well plate and 100 μL of supernatant was collected for measuring luminescence using a plate reader.

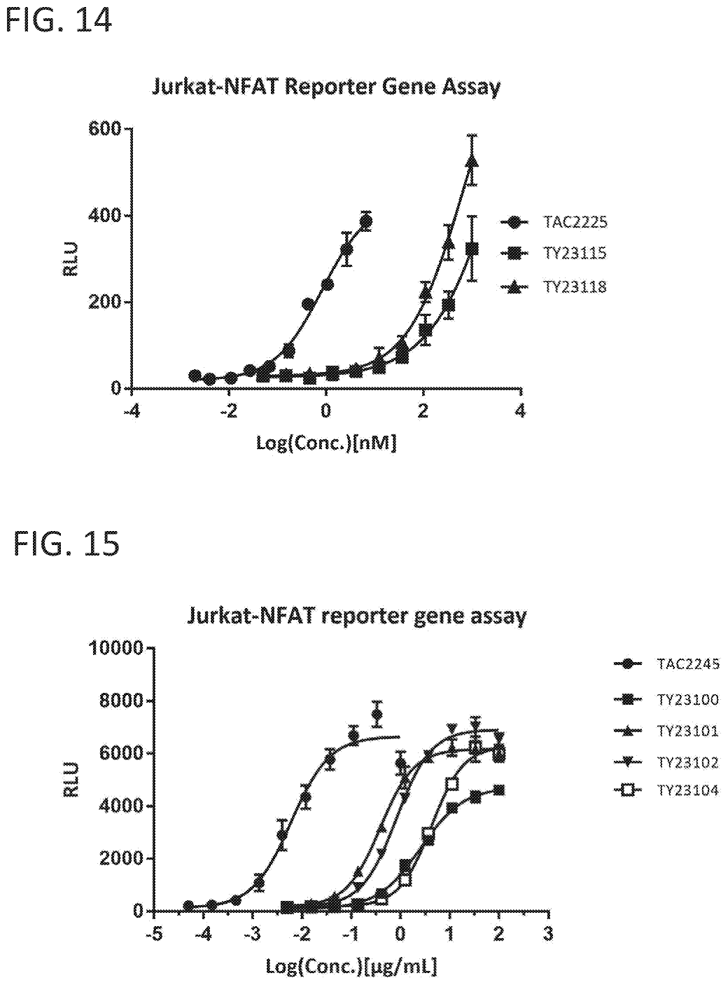

As shown in FIG. 14, the parental CD3 antibody TAC2225 showed the strongest potency (i.e., activated the reporter at the lowest concentration of antibody) compared to the activatable antibodies TY23115 and TY23118. These results indicated that the activatable antibodies TY23115 and TY23118 showed good masking efficiencies.

Jurkat NFAT Reporter Assay of huOKT3 and Activatable huOKT3 Antibodies

The activities of parental (TAC2245) and activatable (TY23100, TY23101, TY23102 and TY23104) huOKT3 anti-CD3 antibodies were tested in a Jurkat NFAT reporter assay.

Jurkat-NFAT-luc2 cells were thawed, washed, and cultured in an incubator. Diluted samples of antibody were prepared in assay medium consisting of RPM 1640 supplemented with 1% FBS. 7.5×10⁴ Jurkat-NFAT-luc2 cells/well were added to a 96-well plate and incubated with FcRIIb-CHO-K1 (3.0×10⁴) and diluted test antibodies for 6 hours. After incubation, 100 μL/well ONE-Glo Luciferase Assay buffer was added to the 96-well plate, and 100 μL supernatant was collected for measuring luminescence using a suitable plate reader.

As shown in FIG. 15, the parental huOKT3 anti-CD3 antibody TAC2245 activated the reporter at a lower concentration relative to the activatable huOKT3 anti-CD3 antibodies TY23100, TY23101, TY23102, and TY23104. This was true for assays performed with FcRIIb crosslinking (data not shown) or without FcRIIb crosslinking (FIG. 15).

### Masking Efficiency of Human/Cynomolgus Cross-Reactive CD3 Activatable Antibodies

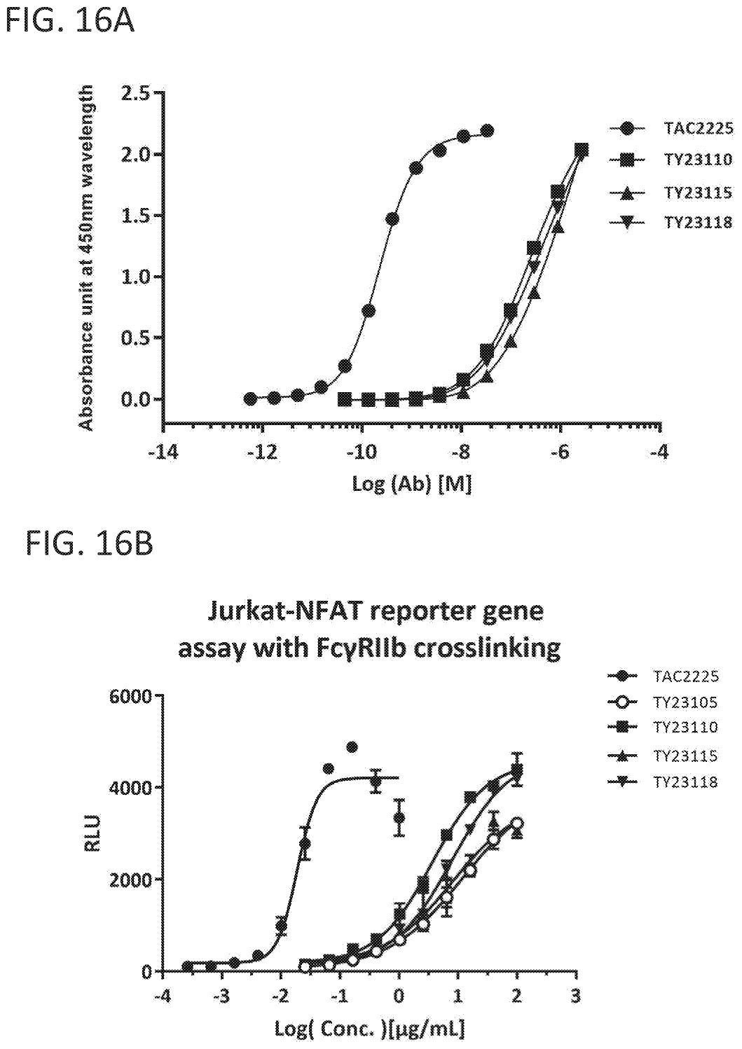

The masking efficiency of TAC2225 and the activatable antibodies derived from TAC2225 (TY23105, TY23110, TY23115 and TY23118) were measured using an ELISA (FIG. 16A) and a Jurkat NFAT reporter assay (FIG. 16B).

For the ELISA, recombinant human CD3δε-hisFc was diluted to 111 g/mL in PBS and coated on a Maxisorp plate at 4° C. overnight. Plates were blocked with PBS supplemented with 3% non-fat milk at 37° C. for 1 hour. After washing, 100 μL of 3-fold serial dilutions of antibodies were added to each well. After incubation at 37° C. for 1 hour, plates were washed four times, and 100 μL HRP conjugated anti-human IgG (Fab specific) (1:6000 dilution) was added to each well. Plates were incubated at 37° C. for 1 hour, washed four times, and then 50 11L TMB substrate solution was added to each well, and the plate was incubated at room temperature. Absorbance at 450 nm was measured after the reactions were stopped with 50 μL H₂SO₄ per well. The EC₅₀ was evaluated by fitting the ELISA data using the asymmetrical sigmoidal (five-parameter logistic equation) model of GraphPad Prism 6 software.

For the Jurkat NFAT reporter assay, Jurkat-NFAT-luc2 cells were thawed, washed and cultured in an incubator. Diluted samples of antibody were prepared in assay medium consisting of RPM 1640 supplemented with 1% FBS as indicated in the figure. 7.5×10⁴ Jurkat-NFAT-luc2 cells/well were added to the 96-well plate and incubated with FcRIIb-CHO-K1 (2.5×10⁴) and diluted test antibodies for 6 hours. After incubation, 100 μL/well ONE-Glo Luciferase Assay buffer was added to the 96-well plate and 100 μL supernatant was collected for measuring luminescence using a plate reader.

The masking efficiency of each activatable antibodies was calculated by dividing the EC₅₀ for binding of the activatable antibody by the EC₅₀ of the parental antibody. As shown in Tables 4-5A, compared with the parental antibody TAC2225, all of the activatable antibodies showed dramatically reduced binding to the antigen. Further, all of the activatable antibodies showed reduced activation of the NFAT reporter.

TABLE 4
Masking efficiency of human/cynomolgus cross-
reactive CD3 activatable antibodies by ELISA

| IgG ID | EC50, nM | Masking efficiency |
|---|---|---|
| TAC2225 | 0.2 | 1 |
| TY23110 | 313.5 | 1339 |
| TY23115 | 19570 | >10000 |
| TY23118 | 864.5 | 3691 |

TABLE 5A
Masking efficiency of human/cynomolgus cross-reactive
CD3 activatable antibodies Jurkat NFAT reporter assay

| IgG ID | EC50, nM | Masking efficiency |
|---|---|---|
| TAC2225 | 0.02 | 1 |
| TY23105 | 11.5 | 611 |
| TY23110 | 3.6 | 191 |
| TY23115 | 7.7 | 409 |
| TY23118 | 7.9 | 419 |

### Example 4. Design and Characterization of Human/Cynomolgus Cross-Reactive Anti-CD3 Antibody Variants

The following example describes the generation of anti-CD3 antibodies from parental antibody SP34. Specifically, anti-CD3 antibodies with low binding affinities were generated.

### Materials and Methods

### Generation of Variants of Anti-CD3 Antibody SP34

The human/cynomolgus cross-reactive anti-CD3 murine antibody SP34 was selected as the basis for designing variant antibodies because SP34's epitope in CD3 is conserved between monkey and human, but not conserved between mice and human.

The sequences of the SP34 heavy chain variable region (VH) and light chain variable region (VL) are provided below, with the CDR sequences bolded, underlined, and italicized.

>SP34 VH
(SEQ ID NO: 374)
EVQLVESGGGLVQPKGSLKLSCAASGFTFNTYAMNWVRQAPGKGLEWVA
RIRSKYNNYATYYADSVKDRFTISRDDSQSILYLQMNNLKTEDTAMYYC
VRHGNFGNSYVSWFAYWGQGTLVTVSS
>SP34 VL
(SEQ ID NO: 375)
QAVVTQESALTTSPGETVTLTCRSSTGAVTTSNYANWVQEKPDHLFTGL
IGGTNKRAPGVPARFSGSLIGDKAALTITGAQTEDEAIYFCALWYSNLW
VFGGGTKLTVL

Specifically, humanized variants of the SP34 VH and VL were used (see International Publication No. WO2014110601A1). Relative to SP34, these humanized sequences contain an amino acid substitution in the CDR-H2 and in the CDR-L1. The amino acid sequences of the humanized VH and VL are set forth in SEQ ID NO: 67 and 68, respectively.

Based on the SP34 CDR sequences, sequences with variant CDRs were designed, as shown in Table 5B, below. Further, variants of the VH and VL sequences of SP34 were designed, as shown in Table 5C, below.

TABLE 5B
Design of anti-CD3 variant CDRs

| | | | SP34 |
|---|---|---|---|
| | | | Variant |
| | SP34 | SP34 | SEQ ID |
| CDR | Amino Acid Sequence | Variant Amino Acid Sequence | NO: |
| CDR- | TYAMN | X₁YAX₂X₃ | 382 |
| H1 | (SEQ ID NO: 376) | X₁: D S T | |
| | | X₂: I L M | |
| | | X₃: N T | |
| CDR- | RIRSKYNNYATYYADSVKD | RIRSKYNNYATYYAX₁X₂VKX₃ | 383 |
| H2 | (SEQ ID NO: 377) | X₁: D E | |
| | | X₂: S T | |
| | | X₃: D G S | |
| CDR- | HGNFGNSYVSWFAY | HGNX₁GX₂SYVSX₃X₄AY | 384 |
| H3 | (SEQ ID NO: 378) | X₁: F Y | |
| | | X₂: N T | |
| | | X₃: W Y | |
| | | X₄: F W | |
| CDR- | RSSTGAVTTSNYAN | X₁SSTGAVTX₂X₃NYX₄N | 385 |
| L1 | (SEQ ID NO: 379) | X₁: A GR | |
| | | X₂: S T | |
| | | X₃: G S | |
| | | X₄: A P V | |
| CDR- | GTNKRAP | GTX₁X₂RAP | 386 |
| L2 | (SEQ ID NO: 380) | X₁: K N | |
| | | X₂: F K | |
| CDR- | ALWYSNLWV | ALWYSX₁X₂WV | 387 |
| L3 | (SEQ ID NO: 381) | X₁: D N T | |
| | | X₂: L R | |

TABLE 5C
Design of anti-CD3 variant variable regions

| | | SP34 Variant |
|---|---|---|
| Variable | | Amino Acid |
| Region | SP34 Variant Amino Acid Sequence | SEQ ID NO: |
| VH | EVQLVESGGGLVX₁PGGSLRLSCAASGFTFX₂X₃YAIX₄W | 388 |
| | VRQAPGKGLEWVX₅RIRSKYNNYATYYAX₆SVKX₇RFTIS | |
| | RDX₈SKNTLYLQX₉NSLRAEDTAVYYCX₁₀RHGNX₁₁GX₁₂ | |
| | SYVSWFAYWGQGTLVTVSS | |
| | X₁: K Q | |
| | X₂: N S | |
| | X₃: S T | |
| | X₄: H N | |
| | X₅: G S | |
| | X₆: D E | |
| | X₇: D G | |
| | X₈: D N | |
| | X₉: I L | |
| | X₁₀: A V | |
| | X₁₁: F Y | |
| | X₁₂: N T | |
| VL | X₁AVVTQEPSLTVSPGGTVTLTCX₂SSTGAVTTSNYX₃NW | 389 |
| | X₄QQKPGQAPRGLIGGTX₅X₆RAPGX₇PARFSGSLLGGKA | |
| | ALTLSGAQPEDEAEYYCALWYSX₈X₉WVFGGGTKLTVL | |
| | X₁: E Q | |
| | X₂: A G P R | |
| | X₃: A P | |
| | X₄: F V | |
| | X₅: K N | |
| | X₆: F K | |
| | X₇: A I T V | |
| | X₈: A D N T | |
| | X₉: H L | |

Exemplary CDR and VH and VL sequences that were generated are provided in Tables 5D-5G below.

TABLE 5D
Amino acid sequences of variant anti-CD3 heavy chain variable region CDRs

| | CDR-H1 | CDR-H2 | CDR-H3 |
|---|---|---|---|
| VH-01 | TYAMN | RIRSKYNNYATYYADSVKD | HGNFGTSYVSWFAY |
| | (SEQ ID NO: 376) | (SEQ ID NO: 377) | (SEQ ID NO: 395) |
| VH-03 | TYAMN | RIRSKYNNYATYYADSVKG | HGNFGTSYVSWFAY |
| | (SEQ ID NO: 376) | (SEQ ID NO: 391) | (SEQ ID NO: 395) |
| VH-04 | TYAMN | RIRSKYNNYATYYADSVKD | HGNFGTSYVSWFAY |
| | (SEQ ID NO: 376) | (SEQ ID NO: 377) | (SEQ ID NO: 395) |
| VH-05 | TYAMN | RIRSKYNNYATYYADSVKD | HGNFGNSYVSYWAY |
| | (SEQ ID NO: 376) | (SEQ ID NO: 377) | (SEQ ID NO: 604) |
| VH-17 | TYAMT | RIRSKYNNYATYYADSVKG | HGNFGNSYVSWFAY |
| | (SEQ ID NO: 600) | (SEQ ID NO: 391) | (SEQ ID NO: 378) |
| VH-19 | TYAMN | RIRSKYNNYATYYADTVKG | HGNFGNSYVSWFAY |
| | (SEQ ID NO: 376) | (SEQ ID NO: 603) | (SEQ ID NO: 378) |
| VH-20 | TYAMN | RIRSKYNNYATYYADSVKG | HGNYGNSYVSWFAY |
| | (SEQ ID NO: 376) | (SEQ ID NO: 391) | (SEQ ID NO: 605) |
| VH-22 | DYAMN | RIRSKYNNYATYYADSVKG | HGNFGNSYVSWFAY |
| | (SEQ ID NO: 601) | (SEQ ID NO: 391) | (SEQ ID NO: 378) |
| VH-23 | TYAIN | RIRSKYNNYATYYADSVKG | HGNFGNSYVSWFAY |
| | (SEQ ID NO: 390) | (SEQ ID NO: 391) | (SEQ ID NO: 378) |
| VH-24 | TYALN | RIRSKYNNYATYYADSVKG | HGNFGNSYVSWFAY |
| | (SEQ ID NO: 602) | (SEQ ID NO: 391) | (SEQ ID NO: 378) |
| VH-27 | TYAIN | RIRSKYNNYATYYADSVKD | HGNFGTSYVSWFAY |
| | (SEQ ID NO: 390) | (SEQ ID NO: 377) | (SEQ ID NO: 395) |
| VH-28 | TYAIN | RIRSKYNNYATYYAESVKD | HGNFGTSYVSWFAY |
| | (SEQ ID NO: 390) | (SEQ ID NO: 392) | (SEQ ID NO: 395) |
| VH-29 | TYAIN | RIRSKYNNYATYYADSVKD | HGNFGNSYVSYWAY |
| | (SEQ ID NO: 390) | (SEQ ID NO: 377) | (SEQ ID NO: 604) |
| VH-30 | TYAIN | RIRSKYNNYATYYAESVKD | HGNFGNSYVSYWAY |
| | (SEQ ID NO: 390) | (SEQ ID NO: 392) | (SEQ ID NO: 604) |
| VH-31 | TYAIN | RIRSKYNNYATYYADSVKG | HGNFGTSYVSWFAY |
| | (SEQ ID NO: 390) | (SEQ ID NO: 391) | (SEQ ID NO: 395) |
| VH-32 | TYAIN | RIRSKYNNYATYYAESVKG | HGNFGTSYVSWFAY |
| | (SEQ ID NO: 390) | (SEQ ID NO: 394) | (SEQ ID NO: 395) |
| VH-33 | TYAIN | RIRSKYNNYATYYADSVKD | HGNFGTSYVSWFAY |
| | (SEQ ID NO: 390) | (SEQ ID NO: 377) | (SEQ ID NO: 395) |
| VH-34 | TYAIN | RIRSKYNNYATYYAESVKD | HGNFGTSYVSWFAY |
| | (SEQ ID NO: 390) | (SEQ ID NO: 392) | (SEQ ID NO: 395) |
| VH-35 | TYAIN | RIRSKYNNYATYYADSVKD | HGNFGTSYVSWFAY |
| | (SEQ ID NO: 390) | (SEQ ID NO: 377) | (SEQ ID NO: 395) |
| VH-36 | TYAIN | RIRSKYNNYATYYAESVKG | HGNFGTSYVSWFAY |
| | (SEQ ID NO: 390) | (SEQ ID NO: 394) | (SEQ ID NO: 395) |
| VH-37 | TYAIN | RIRSKYNNYATYYADSVKG | HGNFGTSYVSWFAY |
| | (SEQ ID NO: 390) | (SEQ ID NO: 391) | (SEQ ID NO: 395) |
| VH-38 | TYAIN | RIRSKYNNYATYYADSVKG | HGNFGNSYVSWFAY |
| | (SEQ ID NO: 390) | (SEQ ID NO: 391) | (SEQ ID NO: 378) |
| VH-39 | TYAIN | RIRSKYNNYATYYADSVKD | HGNFGNSYVSWFAY |
| | (SEQ ID NO: 390) | (SEQ ID NO: 377) | (SEQ ID NO: 378) |

TABLE 5E
Amino acid sequences of variant anti-CD3 light chain variable region CDRs

| | CDR-L1 | CDR-L2 | CDR-L3 |
|---|---|---|---|
| VL-01 | RSSTGAVTTSNYPN | GTKFRAP | ALWYSTLWV |
| | (SEQ ID NO: 606) | (SEQ ID NO: 399) | (SEQ ID NO: 401) |
| VL-02 | GSSTGAVTTSNYPN | GTNKRAP | ALWYSDLWV |
| | (SEQ ID NO: 397) | (SEQ ID NO: 380) | (SEQ ID NO: 400) |
| VL-03 | GSSTGAVTTSNYAN | GTNKRAP | ALWYSTLWV |
| | (SEQ ID NO: 396) | (SEQ ID NO: 380) | (SEQ ID NO: 401) |
| VL-04 | RSSTGAVTTSNYAN | GTKFRAP | ALWYSDLWV |
| | (SEQ ID NO: 398) | (SEQ ID NO: 399) | (SEQ ID NO: 400) |
| VL-05 | GSSTGAVTTSNYAN | GTNKRAP | ALWYSNLWV |
| | (SEQ ID NO: 396) | (SEQ ID NO: 380) | (SEQ ID NO: 381) |
| VL-06 | GSSTGAVTTSNYAN | GTNKRAP | ALWYSNLWV |
| | (SEQ ID NO: 396) | (SEQ ID NO: 380) | (SEQ ID NO: 381) |
| VL-07 | GSSTGAVTTSNYAN | GTNKRAP | ALWYSNRWV |
| | (SEQ ID NO: 396) | (SEQ ID NO: 380) | (SEQ ID NO: 610) |
| VL-08 | GSSTGAVTTSNYAN | GTNKRAP | ALWYSNLWV |
| | (SEQ ID NO: 396) | (SEQ ID NO: 380) | (SEQ ID NO: 381) |
| VL-09 | GSSTGAVTTSNYAN | GTNKRAP | ALWYSNLWV |
| | (SEQ ID NO: 396) | (SEQ ID NO: 380) | (SEQ ID NO: 381) |
| VL-10 | GSSTGAVTTSNYAN | GTNKRAP | ALWYSNLWV |
| | (SEQ ID NO: 396) | (SEQ ID NO: 380) | (SEQ ID NO: 381) |
| VL-11 | GSSTGAVTTSNYAN | GTNKRAP | ALWYSNLWV |
| | (SEQ ID NO: 396) | (SEQ ID NO: 380) | (SEQ ID NO: 381) |
| VL-12 | GSSTGAVTTSNYAN | GTNKRAP | ALWYSNLWV |
| | (SEQ ID NO: 396) | (SEQ ID NO: 380) | (SEQ ID NO: 381) |
| VL-13 | RSSTGAVTTSNYAN | GTNKRAP | ALWYSNLWV |
| | (SEQ ID NO: 398) | (SEQ ID NO: 380) | (SEQ ID NO: 381) |
| VL-14 | ASSTGAVTTSNYAN | GTNKRAP | ALWYSNLWV |
| | (SEQ ID NO: 607) | (SEQ ID NO: 380) | (SEQ ID NO: 381) |
| VL-15 | GSSTGAVTSGNYAN | GTNKRAP | ALWYSNLWV |
| | (SEQ ID NO: 608) | (SEQ ID NO: 380) | (SEQ ID NO: 381) |
| VL-16 | GSSTGAVTTSNYPN | GTNKRAP | ALWYSNLWV |
| | (SEQ ID NO: 397) | (SEQ ID NO: 380) | (SEQ ID NO: 381) |
| VL-17 | GSSTGAVTTSNYVN | GTNKRAP | ALWYSNLWV |
| | (SEQ ID NO: 609) | (SEQ ID NO: 380) | (SEQ ID NO: 381) |
| VL-18 | GSSTGAVTTSNYAN | GTNKRAP | ALWYSNLWV |
| | (SEQ ID NO: 396) | (SEQ ID NO: 380) | (SEQ ID NO: 381) |
| VL-19 | GSSTGAVTTSNYAN | GTNKRAP | ALWYSNLWV |
| | (SEQ ID NO: 396) | (SEQ ID NO: 380) | (SEQ ID NO: 381) |
| VL-20 | GSSTGAVTTSNYAN | GTNKRAP | ALWYSNLWV |
| | (SEQ ID NO: 396) | (SEQ ID NO: 380) | (SEQ ID NO: 381) |
| VL-21 | GSSTGAVTTSNYAN | GTNKRAP | ALWYSNLWV |
| | (SEQ ID NO: 396) | (SEQ ID NO: 380) | (SEQ ID NO: 381) |
| VL-22 | GSSTGAVTTSNYAN | GTNKRAP | ALWYSNLWV |
| | (SEQ ID NO: 396) | (SEQ ID NO: 380) | (SEQ ID NO: 381) |
| VL-23 | GSSTGAVTTSNYAN | GTNKRAP | ALWYSNLWV |
| | (SEQ ID NO: 396) | (SEQ ID NO: 380) | (SEQ ID NO: 381) |
| VL-24 | GSSTGAVTTSNYAN | GTNKRAP | ALWYSNLWV |
| | (SEQ ID NO: 396) | (SEQ ID NO: 380) | (SEQ ID NO: 381) |
| VL-25 | GSSTGAVTTSNYAN | GTNKRAP | ALWYSNLWV |
| | (SEQ ID NO: 396) | (SEQ ID NO: 380) | (SEQ ID NO: 381) |
| VL-26 | GSSTGAVTTSNYAN | GTNKRAP | ALWYSNLWV |
| | (SEQ ID NO: 396) | (SEQ ID NO: 380) | (SEQ ID NO: 381) |
| VL-27 | GSSTGAVTTSNYAN | GTNKRAP | ALWYSNLWV |
| | (SEQ ID NO: 396) | (SEQ ID NO: 380) | (SEQ ID NO: 381) |
| VL-28 | GSSTGAVTTSNYAN | GTNKRAP | ALWYSNLWV |
| | (SEQ ID NO: 396) | (SEQ ID NO: 380) | (SEQ ID NO: 381) |
| VL-29 | GSSTGAVTTSNYAN | GTNKRAP | ALWYSNLWV |
| | (SEQ ID NO: 396) | (SEQ ID NO: 380) | (SEQ ID NO: 381) |
| VL-30 | GSSTGAVTTSNYAN | GTNKRAP | ALWYSNLWV |
| | (SEQ ID NO: 396) | (SEQ ID NO: 380) | (SEQ ID NO: 381) |
| VL-31 | GSSTGAVTTSNYPN | GTNKRAP | ALWYSNLWV |
| | (SEQ ID NO: 397) | (SEQ ID NO: 380) | (SEQ ID NO: 381) |
| VL-32 | GSSTGAVTTSNYAN | GTNKRAP | ALWYSNLWV |
| | (SEQ ID NO: 396) | (SEQ ID NO: 380) | (SEQ ID NO: 381) |

TABLE 5F
Amino acid sequences of variant anti-CD3 VHs

| | | SEQ ID |
|---|---|---|
| | VH Amino Acid Sequence | NO |
| VH-01 | EVQLVESGGGLVQPGGSLRLSCAASGFTFSTYAMNWVRQAPGKGLEWV | 409 |
| | SRIRSKYNNYATYYADSVKDRFTISRDDSKNTLYLQINSLRAEDTAVY | |
| | YCVRHGNFGTSYVSWFAYWGQGTLVTVSS | |
| VH-02 | EVQLVESGGGLVQPGGSLRLSCAASGFTFNTYAMNWVRQAPGKGLEWV | 414 |
| | SRIRSKYNNYATYYADSVKGRFTISRDDSKNTLYLQLNSLRAEDTAVY | |
| | YCVRHGNFGNSYVSWFAYWGQGTLVTVSS | |
| VH-03 | EVQLVESGGGLVKPGGSLRLSCAASGFTFSTYAMNWVRQAPGKGLEWV | 611 |
| | SRIRSKYNNYATYYADSVKGRFTISRDDSKNTLYLQINSLRAEDTAVY | |
| | YCVRHGNFGTSYVSWFAYWGQGTLVTVSS | |
| VH-04 | EVQLVESGGGLVKPGGSLRLSCAASGFTFSTYAMNWVRQAPGKGLEWV | 407 |
| | SRIRSKYNNYATYYADSVKDRFTISRDDSKNTLYLQINSLRAEDTAVY | |
| | YCVRHGNFGTSYVSWFAYWGQGTLVTVSS | |
| VH-05 | EVQLVESGGGLVKPGGSLRLSCAASGFTFSTYAMNWVRQAPGKGLEWV | 612 |
| | SRIRSKYNNYATYYADSVKDRFTISRDDSKNTLYLQINSLRAEDTAVY | |
| | YCVRHGNFGNSYVSYWAYWGQGTLVTVSS | |
| VH-06 | EVQLVESGGGLVKPGGSLRLSCAASGFTFNTYAMNWVRQAPGKGLEWV | 613 |
| | GRIRSKYNNYATYYADSVKGRFTISRDDSKNTLYLQINSLRAEDTAVY | |
| | YCVRHGNFGNSYVSWFAYWGQGTLVTVSS | |
| VH-07 | EVQLVESGGGLVQPGGSLRLSCAASGFTFNSYAMNWVRQAPGKGLEWV | 614 |
| | GRIRSKYNNYATYYADSVKGRFTISRDDSKNTLYLQMNSLRAEDTAVY | |
| | YCVRHGNFGNSYVSWFAYWGQGTLVTVSS | |
| VH-08 | EVQLVESGGGLVQPGGSLRLSCAASGFTFNTYAMNWVRQAPGKGLEWV | 615 |
| | GRIRSKYNNYATYYADSVKSRFTISRDDSKNTLYLQMNSLRAEDTAVY | |
| | YCVRHGNFGNSYVSWFAYWGQGTLVTVSS | |
| VH-09 | EVQLVESGGGLVQPGGSLRLSCAASGFTFNTYAMNWVRQAPGKGLEWV | 616 |
| | GRIRSKYNNYATYYADSVKGRFTISRDDSRSTLYLQMNSLRAEDTAVY | |
| | YCVRHGNFGNSYVSWFAYWGQGTLVTVSS | |
| VH-10 | EVQLVESGGGLVQPGGSLRLSCAASGFTFNTYAMNWVRQAPGKGLEWV | 617 |
| | GRIRSKYNNYATYYADSVKGRFTISRDDSQSTLYLQMNSLRAEDTAVY | |
| | YCVRHGNFGNSYVSWFAYWGQGTLVTVSS | |
| VH-11 | EVQLVESGGGLVQPGGSLRLSCAASGFTFNTYAMNWVRQAPGKGLEWV | 618 |
| | GRIRSKYNNYATYYADSVKGRFTISRDDSKNSIYLQMNSLRAEDTAVY | |
| | YCVRHGNFGNSYVSWFAYWGQGTLVTVSS | |
| VH-12 | EVQLVESGGGLVQPGGSLRLSCAASGFTFNTYAMNWVRQAPGKGLEWV | 619 |
| | GRIRSKYNNYATYYADSVKGRFTISRDDSKNTLYLQMNTLRAEDTAVY | |
| | YCVRHGNFGNSYVSWFAYWGQGTLVTVSS | |
| VH-13 | EVQLVETGGGLVQPGGSLRLSCAASGFTFNTYAMNWVRQAPGKGLEWV | 620 |
| | GRIRSKYNNYATYYADSVKGRFTISRDDSKNTLYLQMNSLRAEDTAVY | |
| | YCVRHGNFGNSYVSWFAYWGQGTLVTVSS | |
| VH-14 | EVQLVESGGGLIQPGGSLRLSCAASGFTFNTYAMNWVRQAPGKGLEWV | 621 |
| | GRIRSKYNNYATYYADSVKGRFTISRDDSKNTLYLQMNSLRAEDTAVY | |
| | YCVRHGNFGNSYVSWFAYWGQGTLVTVSS | |
| VH-15 | EVQLVESGGGLLQPGGSLRLSCAASGFTFNTYAMNWVRQAPGKGLEWV | 622 |
| | GRIRSKYNNYATYYADSVKGRFTISRDDSKNTLYLQMNSLRAEDTAVY | |
| | YCVRHGNFGNSYVSWFAYWGQGTLVTVSS | |
| VH-16 | EVQLVESGGGLVQPGGSLRLSCAASGFTFNTYAMNWVRQAPGKGLEWV | 623 |
| | GRIRSKYNNYATYYADSVKGRFTISRDDSKNTLYLQMNSLKTEDTAVY | |
| | YCVRHGNFGNSYVSWFAYWGQGTLVTVSS | |
| VH-17 | EVQLVESGGGLVQPGGSLRLSCAASGFTFNTYAMTWVRQAPGKGLEWV | 624 |
| | GRIRSKYNNYATYYADSVKGRFTISRDDSKNTLYLQMNSLRAEDTAVY | |
| | YCVRHGNFGNSYVSWFAYWGQGTLVTVSS | |
| VH-18 | EVQLVESGGGLVQPGGSLRLSCAASGFTFNTYAMNWVRQAPGQGLEWV | 625 |
| | GRIRSKYNNYATYYADSVKGRFTISRDDSKNTLYLQMNSLRAEDTAVY | |
| | YCVRHGNFGNSYVSWFAYWGQGTLVTVSS | |
| VH-19 | EVQLVESGGGLVQPGGSLRLSCAASGFTFNTYAMNWVRQAPGKGLEWV | 626 |
| | GRIRSKYNNYATYYADTVKGRFTISRDDSKNTLYLQMNSLRAEDTAVY | |
| | YCVRHGNFGNSYVSWFAYWGQGTLVTVSS | |
| VH-20 | EVQLVESGGGLVQPGGSLRLSCAASGFTFNTYAMNWVRQAPGKGLEWV | 627 |
| | GRIRSKYNNYATYYADSVKGRFTISRDDSKNTLYLQMNSLRAEDTAVY | |
| | YCVRHGNYGNSYVSWFAYWGQGTLVTVSS | |
| VH-21 | EVQLVESGGGLVQPGGSLRLSCAASGFTFSSYAMNWVRQAPGKGLEWV | 628 |
| | GRIRSKYNNYATYYADSVKGRFTISRDDSKNTLYLQMNSLRAEDTAVY | |
| | YCVRHGNFGNSYVSWFAYWGQGTLVTVSS | |
| VH-22 | EVQLVESGGGLVQPGGSLRLSCAASGFTFSDYAMNWVRQAPGKGLEWV | 629 |
| | GRIRSKYNNYATYYADSVKGRFTISRDDSKNTLYLQMNSLRAEDTAVY | |
| | YCVRHGNFGNSYVSWFAYWGQGTLVTVSS | |
| VH-23 | EVQLVESGGGLVQPGGSLRLSCAASGFTFNTYAINWVRQAPGKGLEWV | 630 |
| | GRIRSKYNNYATYYADSVKGRFTISRDDSKNTLYLQMNSLRAEDTAVY | |
| | YCVRHGNFGNSYVSWFAYWGQGTLVTVSS | |
| VH-24 | EVQLVESGGGLVQPGGSLRLSCAASGFTFNTYALNWVRQAPGKGLEWV | 631 |
| | GRIRSKYNNYATYYADSVKGRFTISRDDSKNTLYLQMNSLRAEDTAVY | |
| | YCVRHGNFGNSYVSWFAYWGQGTLVTVSS | |
| VH-25 | EVQLVESGGGLVQPGGSLRLSCAASGFTFNTYAMNWVRQAPGKGLEWV | 632 |
| | GRIRSKYNNYATYYADSVKGRFTISRDDSKNTLYLQINSLRAEDTAVY | |
| | YCVRHGNFGNSYVSWFAYWGQGTLVTVSS | |
| VH-26 | EVQLVESGGGLVQPGGSLRLSCAASGFTFNTYAMNWVRQAPGKGLEWV | 633 |
| | GRIRSKYNNYATYYADSVKGRFTISRDDSKNTLYLQLNSLRAEDTAVY | |
| | YCVRHGNFGNSYVSWFAYWGQGTLVTVSS | |
| VH-27 | EVQLVESGGGLVQPGGSLRLSCAASGFTFSTYAINWVRQAPGKGLEWV | 634 |
| | SRIRSKYNNYATYYADSVKDRFTISRDDSKNTLYLQINSLRAEDTAVY | |
| | YCVRHGNFGTSYVSWFAYWGQGTLVTVSS | |
| VH-28 | EVQLVESGGGLVQPGGSLRLSCAASGFTFSTYAINWVRQAPGKGLEWV | 402 |
| | SRIRSKYNNYATYYAESVKDRFTISRDDSKNTLYLQINSLRAEDTAVY | |
| | YCVRHGNFGTSYVSWFAYWGQGTLVTVSS | |
| VH-29 | EVQLVESGGGLVKPGGSLRLSCAASGFTFSTYAINWVRQAPGKGLEWV | 635 |
| | SRIRSKYNNYATYYADSVKDRFTISRDDSKNTLYLQINSLRAEDTAVY | |
| | YCVRHGNFGNSYVSYWAYWGQGTLVTVSS | |
| VH-30 | EVQLVESGGGLVKPGGSLRLSCAASGFTFSTYAINWVRQAPGKGLEWV | 636 |
| | SRIRSKYNNYATYYAESVKDRFTISRDDSKNTLYLQINSLRAEDTAVY | |
| | YCVRHGNFGNSYVSYWAYWGQGTLVTVSS | |
| VH-31 | EVQLVESGGGLVKPGGSLRLSCAASGFTFSTYAINWVRQAPGKGLEWV | 637 |
| | SRIRSKYNNYATYYADSVKGRFTISRDDSKNTLYLQINSLRAEDTAVY | |
| | YCVRHGNFGTSYVSWFAYWGQGTLVTVSS | |
| VH-32 | EVQLVESGGGLVKPGGSLRLSCAASGFTFSTYAINWVRQAPGKGLEWV | 638 |
| | SRIRSKYNNYATYYAESVKGRFTISRDDSKNTLYLQINSLRAEDTAVY | |
| | YCVRHGNFGTSYVSWFAYWGQGTLVTVSS | |
| VH-33 | EVQLVESGGGLVKPGGSLRLSCAASGFTFSTYAINWVRQAPGKGLEWV | 639 |
| | SRIRSKYNNYATYYADSVKDRFTISRDDSKNTLYLQINSLRAEDTAVY | |
| | YCVRHGNFGTSYVSWFAYWGQGTLVTVSS | |
| VH-34 | EVQLVESGGGLVKPGGSLRLSCAASGFTFSTYAINWVRQAPGKGLEWV | 640 |
| | SRIRSKYNNYATYYAESVKDRFTISRDDSKNTLYLQINSLRAEDTAVY | |
| | YCVRHGNFGTSYVSWFAYWGQGTLVTVSS | |
| VH-35 | EVQLVESGGGLVQPGGSLRLSCAASGFTFNTYAINWVRQAPGKGLEWV | 405 |
| | SRIRSKYNNYATYYADSVKDRFTISRDDSKNTLYLQINSLRAEDTAVY | |
| | YCVRHGNFGTSYVSWFAYWGQGTLVTVSS | |
| VH-36 | EVQLVESGGGLVQPGGSLRLSCAASGFTFNTYAINWVRQAPGKGLEWV | 410 |
| | GRIRSKYNNYATYYAESVKGRFTISRDDSKNTLYLQINSLRAEDTAVY | |
| | YCVRHGNFGTSYVSWFAYWGQGTLVTVSS | |
| VH-37 | EVQLVESGGGLVQPGGSLRLSCAASGFTFNTYAINWVRQAPGKGLEWV | 412 |
| | GRIRSKYNNYATYYADSVKGRFTISRDDSKNTLYLQINSLRAEDTAVY | |
| | YCVRHGNFGTSYVSWFAYWGQGTLVTVSS | |
| VH-38 | EVQLVESGGGLVQPGGSLRLSCAASGFTFNTYAINWVRQAPGKGLEWV | 416 |
| | GRIRSKYNNYATYYADSVKGRFTISRDDSKNTLYLQINSLRAEDTAVY | |
| | YCVRHGNFGNSYVSWFAYWGQGTLVTVSS | |
| VH-39 | EVQLVESGGGLVQPGGSLRLSCAASGFTFNTYAINWVRQAPGKGLEWV | 415 |
| | GRIRSKYNNYATYYADSVKDRFTISRDDSKNTLYLQINSLRAEDTAVY | |
| | YCVRHGNFGNSYVSWFAYWGQGTLVTVSS | |

TABLE 5G
Amino acid sequences of variant anti-CD3 VLs

| | | SEQ ID |
|---|---|---|
| | VL Amino Acid Sequence | NO |
| VL-01 | EAVVTQEPSLTVSPGGTVTLTCRSSTGAVTTSNYPNWVQQKPGQAPRG | 641 |
| | LIGGTKFRAPGTPARFSGSLLGGKAALTLSGAQPEDEAEYYCALWYST | |
| | LWVFGGGTKLTVL | |
| VL-02 | EAVVTQEPSLTVSPGGTVTLTCGSSTGAVTTSNYPNWVQQKPGQAPRG | 403 |
| | LIGGTNKRAPGTPARFSGSLLGGKAALTLSGAQPEDEAEYYCALWYSD | |
| | LWVFGGGTKLTVL | |
| VL-03 | QAVVTQEPSLTVSPGGTVTLTCGSSTGAVTTSNYANWVQQKPGQAPRG | 404 |
| | LIGGTNKRAPGTPARFSGSLLGGKAALTLSGAQPEDEAEYYCALWYST | |
| | LWVFGGGTKLTVL | |
| VL-04 | EAVVTQEPSLTVSPGGTVTLTCRSSTGAVTTSNYANWVQQKPGQAPRG | 408 |
| | LIGGTKFRAPGVPARFSGSLLGGKAALTLSGAQPEDEAEYYCALWYSD | |
| | LWVFGGGTKLTVL | |
| VL-05 | EAVVTQEPSLTVSPGGTVTLTCGSSTGAVTTSNYANWVQQKPGQAPRG | 642 |
| | LIGGTNKRAPGTPARFSGSLLGGKAALTLSGAQPEDEAEYYCALWYSN | |
| | LWVFGGGTKLTVL | |
| VL-06 | EAVVTQEPSLTVSPGGTVTLTCGSSTGAVTTSNYANWVQQKPGQAPRG | 643 |
| | LIGGTNKRAPGVPARFSGSLLGGKAALTLSGAQPEDEAEYYCALWYSN | |
| | LWVFGGGTKLTVL | |
| VL-07 | QTVVTQEPSLTVSPGGTVTLTCGSSTGAVTTSNYANWVQQKPGQAPRG | 644 |
| | LIGGTNKRAPGVPARFSGSLLGGKAALTLSGAQPEDEAEYYCALWYSN | |
| | RWVFGGGTKLTVL | |
| VL-08 | QAVVTQESALTVSPGGTVTLTCGSSTGAVTTSNYANWVQQKPGQAPRG | 645 |
| | LIGGTNKRAPGVPARFSGSLLGGKAALTLSGAQPEDEAEYYCALWYSN | |
| | LWVFGGGTKLTVL | |
| VL-09 | QAVVTQESTLTVSPGGTVTLTCGSSTGAVTTSNYANWVQQKPGQAPRG | 646 |
| | LIGGTNKRAPGVPARFSGSLLGGKAALTLSGAQPEDEAEYYCALWYSN | |
| | LWVFGGGTKLTVL | |
| VL-10 | QAVVTQEPSLSTSPGGTVTLTCGSSTGAVTTSNYANWVQQKPGQAPRG | 647 |
| | LIGGTNKRAPGVPARFSGSLLGGKAALTLSGAQPEDEAEYYCALWYSN | |
| | LWVFGGGTKLTVL | |
| VL-11 | QAVVTQEPSLSASPGGTVTLTCGSSTGAVTTSNYANWVQQKPGQAPRG | 648 |
| | LIGGTNKRAPGVPARFSGSLLGGKAALTLSGAQPEDEAEYYCALWYSN | |
| | LWVFGGGTKLTVL | |
| VL-12 | QAVVTQEPSLTVSPGVTVTLTCGSSTGAVTTSNYANWVQQKPGQAPRG | 649 |
| | LIGGTNKRAPGVPARFSGSLLGGKAALTLSGAQPEDEAEYYCALWYSN | |
| | LWVFGGGTKLTVL | |
| VL-13 | QAVVTQEPSLTVSPGGTVTLTCRSSTGAVTTSNYANWVQQKPGQAPRG | 650 |
| | LIGGTNKRAPGVPARFSGSLLGGKAALTLSGAQPEDEAEYYCALWYSN | |
| | LWVFGGGTKLTVL | |
| VL-14 | QAVVTQEPSLTVSPGGTVTLTCASSTGAVTTSNYANWVQQKPGQAPRG | 651 |
| | LIGGTNKRAPGVPARFSGSLLGGKAALTLSGAQPEDEAEYYCALWYSN | |
| | LWVFGGGTKLTVL | |
| VL-15 | QAVVTQEPSLTVSPGGTVTLTCGSSTGAVTSGNYANWVQQKPGQAPRG | 652 |
| | LIGGTNKRAPGVPARFSGSLLGGKAALTLSGAQPEDEAEYYCALWYSN | |
| | LWVFGGGTKLTVL | |
| VL-16 | QAVVTQEPSLTVSPGGTVTLTCGSSTGAVTTSNYPNWVQQKPGQAPRG | 411 |
| | LIGGTNKRAPGVPARFSGSLLGGKAALTLSGAQPEDEAEYYCALWYSN | |
| | LWVFGGGTKLTVL | |
| VL-17 | QAVVTQEPSLTVSPGGTVTLTCGSSTGAVTTSNYVNWVQQKPGQAPRG | 653 |
| | LIGGTNKRAPGVPARFSGSLLGGKAALTLSGAQPEDEAEYYCALWYSN | |
| | LWVFGGGTKLTVL | |
| VL-18 | QAVVTQEPSLTVSPGGTVTLTCGSSTGAVTTSNYANWVQQKPDHLFRG | 654 |
| | LIGGTNKRAPGVPARFSGSLLGGKAALTLSGAQPEDEAEYYCALWYSN | |
| | LWVFGGGTKLTVL | |
| VL-19 | QAVVTQEPSLTVSPGGTVTLTCGSSTGAVTTSNYANWVQQKPGQAPRG | 655 |
| | LIGGTNKRAPWTPARFSGSLLGGKAALTLSGAQPEDEAEYYCALWYSN | |
| | LWVFGGGTKLTVL | |
| VL-20 | QAVVTQEPSLTVSPGGTVTLTCGSSTGAVTTSNYANWVQQKPGQAPRG | 656 |
| | LIGGTNKRAPWSPARFSGSLLGGKAALTLSGAQPEDEAEYYCALWYSN | |
| | LWVFGGGTKLTVL | |
| VL-21 | QAVVTQEPSLTVSPGGTVTLTCGSSTGAVTTSNYANWVQQKPGQAPRG | 657 |
| | LIGGTNKRAPGVPARFSGSLIGGKAALTLSGAQPEDEAEYYCALWYSN | |
| | LWVFGGGTKLTVL | |
| VL-22 | QAVVTQEPSLTVSPGGTVTLTCGSSTGAVTTSNYANWVQQKPGQAPRG | 658 |
| | LIGGTNKRAPGVPARFSGSLLGDKAALTLSGAQPEDEAEYYCALWYSN | |
| | LWVFGGGTKLTVL | |
| VL-23 | QAVVTQEPSLTVSPGGTVTLTCGSSTGAVTTSNYANWVQQKPGQAPRG | 659 |
| | LIGGTNKRAPGVPARFSGSLLGNKAALTLSGAQPEDEAEYYCALWYSN | |
| | LWVFGGGTKLTVL | |
| VL-24 | QAVVTQEPSLTVSPGGTVTLTCGSSTGAVTTSNYANWVQQKPGQAPRG | 660 |
| | LIGGTNKRAPGVPARFSGSLLGGKAALTITGAQPEDEAEYYCALWYSN | |
| | LWVFGGGTKLTVL | |
| VL-25 | QAVVTQEPSLTVSPGGTVTLTCGSSTGAVTTSNYANWVQQKPGQAPRG | 661 |
| | LIGGTNKRAPGVPARFSGSLLGGKAALTLSGVQPEDEAEYYCALWYSN | |
| | LWVFGGGTKLTVL | |
| VL-26 | QAVVTQEPSLTVSPGGTVTLTCGSSTGAVTTSNYANWVQQKPGQAPRG | 662 |
| | LIGGTNKRAPGVPARFSGSLLGGKAALTLSGAQPEDESDYYCALWYSN | |
| | LWVFGGGTKLTVL | |
| VL-27 | QAVVTQEPSLTVSPGGTVTLTCGSSTGAVTTSNYANWVQQKPGQAPRG | 663 |
| | LIGGTNKRAPGVPARFSGSLLGGKAALTLSGAQPEDESQYYCALWYSN | |
| | LWVFGGGTKLTVL | |
| VL-28 | QAVVTQEPSLTVSPGGTVTLTCGSSTGAVTTSNYANWVQQKPGQAPRG | 664 |
| | LIGGTNKRAPGVPARFSGSIIGGKAALTLSGAQPEDEAEYYCALWYSN | |
| | LWVFGGGTKLTVL | |
| VL-29 | QAVVTQEPSLTVSPGGTVTLTCGSSTGAVTTSNYANWVQQKPGQAPRG | 665 |
| | LIGGTNKRAPGVPARFSGSLLGGKAALTLSGAQADDEAEYYCALWYSN | |
| | LWVFGGGTKLTVL | |
| VL-30 | QAVVTQEPSLTVSPGGTVTLTCGSSTGAVTTSNYANWVQQKPGEAPRG | 666 |
| | LIGGTNKRAPGVPARFSGSLLGGKAALTLSGAQPEDEAEYYCALWYSN | |
| | LWVFGGGTKLTVL | |
| VL-31 | QAVVTQEPSLTVSPGGTVTLTCGSSTGAVTTSNYPNWVQQKPGQAPRG | 406 |
| | LIGGTNKRAPGTPARFSGSLLGGKAALTLSGAQPEDEAEYYCALWYSN | |
| | LWVFGGGTKLTVL | |
| VL-32 | QAVVTQEPSLTVSPGGTVTLTCGSSTGAVTTSNYANWVQQKPGQAPRG | 413 |
| | LIGGTNKRAPGTPARFSGSLLGGKAALTLSGAQPEDEAEYYCALWYSN | |
| | LWVFGGGTKLTVL | |

14 unique variants with various combinations of one or more mutations in heavy chain variable regions were generated, and 5 unique variants with various combinations of one or more mutations in light chain variable regions were generated from SP34. IgG1 heavy and light chains with combinations of the mutated variable domains were cloned into the mammalian expression vector pcDNA3.3 (Thermo Fisher Scientific). 50 of IgG sequences were identified and confirmed in further Jurkat full dose binding and Jurkat-NFAT reporter gene assay, as described below.

Certain variants of SP34 were generated and placed in a bispecific format with the anti-HER2 light chain (“LC1”) according to SEQ ID NO: 112, and the anti-HER2 heavy chain (“HC1”) according to SEQ ID NO: 113. The anti-CD3 heavy chains (“HC2”) of these antibodies are shown in Table 5H, below. Each heavy chain in Table 5H comprises, from N- to C-terminus, a VL (bolded), a linker (the amino acid sequence GGGGSGGGGSGGGGSGGGGS), a VH (italicized), and two CH domains. The CDRs are bolded, underlined, and italicized.

TABLE 5H
Anti-CD3 antibody heavy chains

| | | SEQ |
|---|---|---|
| | | ID |
| IgG ID | Anti-CD3 Heavy Chain Amino Acid Sequence | NO |
| TY25520 | EAVVTQEPSLTVSPGGTVTLTCRSSTGAVTTSNYANWVQQKPGQAPRGLIGGT | 673 |
| | KFRAPGVPSRFSGSLLGGKAALTLSGAQPEDEAEYYCALWYSDHWVFGGGTKL | |
| | TVLGGGGSGGGGSGGGGSGGGGSEVQLVESGGGLVQPGGSLRLSCAASGFTFS | |
| | SYAINWVRQAPGKGLEWVGRIRSKYNNYATYYADSVKGRFTISRDDSKNTLYL | |
| | QINSLRAEDTAVYYCARHGNFGTSYVSYWAYWGQGTLVTVSSEPKSSDKTHTC | |
| | PPCPAPELLGGPSVFLFPPKPKDTLMISRTPEVTCVVVDVSHEDPEVKFNWYV | |
| | DGVEVHNAKTKPREEQYASTYRVVSVLTVLHQDWLNGKEYKCKVSNKALPAPI | |
| | EKTISKAKGQPREPQVYTDPPSRDELTKNQVSLTCLVDGFYPSDIAVEWESNG | |
| | QPENCYKTTPPVLDSDGSFFLYSKLTVDKSRWQQGNVFSCSVMHEALHNHYTQ | |
| | DSLSLSPGK | |
| TY25521 | QAVVTQEPSLTVSPGGTVTLTCPSSTGAVTTSNYANWVQQKPGQAPRGLIGGT | 674 |
| | KFRAPGTPARFSGSLLGGKAALTLSGAQPEDEAEYYCALWYSDHWVFGGGTKL | |
| | TVLGGGGSGGGGSGGGGSGGGGSEVQLVESGGGLVKPGGSLRLSCAASGFTFS | |
| | SYAINWVRQAPGKGLEWVGRIRSKYNNYATYYADSVKDRFTISRDDSKNTLYL | |
| | QLNSLRAEDTAVYYCVRHGNFGTSYVSYWAYWGQGTLVTVSSEPKSSDKTHTC | |
| | PPCPAPELLGGPSVFLFPPKPKDTLMISRTPEVTCVVVDVSHEDPEVKFNWYV | |
| | DGVEVHNAKTKPREEQYASTYRVVSVLTVLHQDWLNGKEYKCKVSNKALPAPI | |
| | EKTISKAKGQPREPQVYTDPPSRDELTKNQVSLTCLVDGFYPSDIAVEWESNG | |
| | QPENCYKTTPPVLDSDGSFFLYSKLTVDKSRWQQGNVFSCSVMHEALHNHYTQ | |
| | DSLSLSPGK | |
| TY25523 | QAVVTQEPSLTVSPGGTVTLTCGSSTGAVTTSNYANWVQQKPGQAPRGLIGGT | 675 |
| | NKRAPGVPARFSGSLLGGKAALTLSGAQPEDEAEYYCALWYSNHWVFGGGTKL | |
| | TVLGGGGSGGGGSGGGGSGGGGSEVQLVESGGGLVQPGGSLRLSCAASGFTFS | |
| | SYAINWVRQAPGKGLEWVGRIRSKYNNYATYYAESVKGRFTISRDDSKNTLYL | |
| | QINSLRAEDTAVYYCVRHGNYGTSYVSYWAYWGQGTLVTVSSEPKSSDKTHTC | |
| | PPCPAPELLGGPSVFLFPPKPKDTLMISRTPEVTCVVVDVSHEDPEVKFNWYV | |
| | DGVEVHNAKTKPREEQYASTYRVVSVLTVLHQDWLNGKEYKCKVSNKALPAPI | |
| | EKTISKAKGQPREPQVYTDPPSRDELTKNQVSLTCLVDGFYPSDIAVEWESNG | |
| | QPENCYKTTPPVLDSDGSFFLYSKLTVDKSRWQQGNVFSCSVMHEALHNHYTQ | |
| | DSLSLSPGK | |
| TY25524 | EAVVTQEPSLTVSPGGTVTLTCGSSTGAVTTSNYANWVQQKPGQAPRGLIGGT | 676 |
| | KFRAPGIPARFSGSLLGGKAALTLSGAQPEDEAEYYCALWYSDHWVFGGGTKL | |
| | TVLGGGGSGGGGSGGGGSGGGGSEVQLVESGGGLVQPGGSLRLSCAASGFTFS | |
| | TYAINWVRQAPGKGLEWVGRIRSKYNNYATYYAESVKGRFTISRDDSKNTLYL | |
| | QINSLRAEDTAVYYCARHGNFGTSYVSWFAYWGQGTLVTVSSEPKSSDKTHTC | |
| | PPCPAPELLGGPSVFLFPPKPKDTLMISRTPEVTCVVVDVSHEDPEVKFNWYV | |
| | DGVEVHNAKTKPREEQYASTYRVVSVLTVLHQDWLNGKEYKCKVSNKALPAPI | |
| | EKTISKAKGQPREPQVYTDPPSRDELTKNQVSLTCLVDGFYPSDIAVEWESNG | |
| | QPENCYKTTPPVLDSDGSFFLYSKLTVDKSRWQQGNVFSCSVMHEALHNHYTQ | |
| | DSLSLSPGK | |
| TY25525 | QAVVTQEPSLTVSPGGTVTLTCGSSTGAVTTSNYPNWVQQKPGQAPRGLIGGT | 677 |
| | NKRAPGVPARFSGSLLGGKAALTLSGAQPEDEAEYYCALWYSNLWVFGGGTKL | |
| | TVLGGGGSGGGGSGGGGSGGGGSEVQLVESGGGLVKPGGSLRLSCAASGFTFS | |
| | SYAINWVRQAPGKGLEWVGRIRSKYNNYATYYAESVKGRFTISRDDSKNTLYL | |
| | QINSLRAEDTAVYYCVRHGNFGNSYVSYWAYWGQGTLVTVSSEPKSSDKTHTC | |
| | PPCPAPELLGGPSVFLFPPKPKDTLMISRTPEVTCVVVDVSHEDPEVKFNWYV | |
| | DGVEVHNAKTKPREEQYASTYRVVSVLTVLHQDWLNGKEYKCKVSNKALPAPI | |
| | EKTISKAKGQPREPQVYTDPPSRDELTKNQVSLTCLVDGFYPSDIAVEWESNG | |
| | QPENCYKTTPPVLDSDGSFFLYSKLTVDKSRWQQGNVFSCSVMHEALHNHYTQ | |
| | DSLSLSPGK | |
| TY25526 | EAVVTQEPSLTVSPGGTVTLTCGSSTGAVTTSNYPNWVQQKPGQAPRGLIGGT | 678 |
| | KFRAPGIPARFSGSLLGGKAALTLSGAQPEDEAEYYCALWYSNHWVFGGGTKL | |
| | TVLGGGGSGGGGSGGGGSGGGGSEVQLVESGGGLVQPGGSLRLSCAASGFTFS | |
| | TYAINWVRQAPGKGLEWVSRIRSKYNNYATYYAESVKGRFTISRDDSKNTLYL | |
| | QINSLRAEDTAVYYCVRHGNFGTSYVSYWAYWGQGTLVTVSSEPKSSDKTHTC | |
| | PPCPAPELLGGPSVFLFPPKPKDTLMISRTPEVTCVVVDVSHEDPEVKFNWYV | |
| | DGVEVHNAKTKPREEQYASTYRVVSVLTVLHQDWLNGKEYKCKVSNKALPAPI | |
| | EKTISKAKGQPREPQVYTDPPSRDELTKNQVSLTCLVDGFYPSDIAVEWESNG | |
| | QPENCYKTTPPVLDSDGSFFLYSKLTVDKSRWQQGNVFSCSVMHEALHNHYTQ | |
| | DSLSLSPGK | |
| TY25527 | EAVVTQEPSLTVSPGGTVTLTCGSSTGAVTTSNYANWVQQKPGQAPRGLIGGT | 679 |
| | KFRAPGAPARFSGSLLGGKAALTLSGAQPEDEAEYYCALWYSNHWVFGGGTKL | |
| | TVLGGGGSGGGGSGGGGSGGGGSEVQLVESGGGLVKPGGSLRLSCAASGFTFS | |
| | TYAINWVRQAPGKGLEWVGRIRSKYNNYATYYADSVKGRFTISRDNSKNTLYL | |
| | QLNSLRAEDTAVYYCARHGNFGNSYVSYWAYWGQGTLVTVSSEPKSSDKTHTC | |
| | PPCPAPELLGGPSVFLFPPKPKDTLMISRTPEVTCVVVDVSHEDPEVKFNWYV | |
| | DGVEVHNAKTKPREEQYASTYRVVSVLTVLHQDWLNGKEYKCKVSNKALPAPI | |
| | EKTISKAKGQPREPQVYTDPPSRDELTKNQVSLTCLVDGFYPSDIAVEWESNG | |
| | QPENCYKTTPPVLDSDGSFFLYSKLTVDKSRWQQGNVFSCSVMHEALHNHYTQ | |
| | DSLSLSPGK | |
| TY25528 | QAVVTQEPSLTVSPGGTVTLTCGSSTGAVTTSNYPNWVQQKPGQAPRGLIGGT | 680 |
| | KFRAPGAPARFSGSLLGGKAALTLSGAQPEDEAEYYCALWYSDHWVFGGGTKL | |
| | TVLGGGGSGGGGSGGGGSGGGGSEVQLVESGGGLVQPGGSLRLSCAASGFTFS | |
| | SYAINWVRQAPGKGLEWVGRIRSKYNNYATYYADSVKDRFTISRDNSKNTLYL | |
| | QLNSLRAEDTAVYYCVRHGNFGNSYVSYWAYWGQGTLVTVSSEPKSSDKTHTC | |
| | PPCPAPELLGGPSVFLFPPKPKDTLMISRTPEVTCVVVDVSHEDPEVKFNWYV | |
| | DGVEVHNAKTKPREEQYASTYRVVSVLTVLHQDWLNGKEYKCKVSNKALPAPI | |
| | EKTISKAKGQPREPQVYTDPPSRDELTKNQVSLTCLVDGFYPSDIAVEWESNG | |
| | QPENCYKTTPPVLDSDGSFFLYSKLTVDKSRWQQGNVFSCSVMHEALHNHYTQ | |
| | DSLSLSPGK | |
| TY25529 | EAVVTQEPSLTVSPGGTVTLTCGSSTGAVTTSNYANWVQQKPGQAPRGLIGGT | 681 |
| | NKRAPGVPARFSGSLLGGKAALTLSGAQPEDEAEYYCALWYSDLWVFGGGTKL | |
| | TVLGGGGSGGGGSGGGGSGGGGSEVQLVESGGGLVKPGGSLRLSCAASGFTFS | |
| | SYAINWVRQAPGKGLEWVGRIRSKYNNYATYYADSVKGRFTISRDDSKNTLYL | |
| | QINSLRAEDTAVYYCVRHGNYGTSYVSYWAYWGQGTLVTVSSEPKSSDKTHTC | |
| | PPCPAPELLGGPSVFLFPPKPKDTLMISRTPEVTCVVVDVSHEDPEVKFNWYV | |
| | DGVEVHNAKTKPREEQYASTYRVVSVLTVLHQDWLNGKEYKCKVSNKALPAPI | |
| | EKTISKAKGQPREPQVYTDPPSRDELTKNQVSLTCLVDGFYPSDIAVEWESNG | |
| | QPENCYKTTPPVLDSDGSFFLYSKLTVDKSRWQQGNVFSCSVMHEALHNHYTQ | |
| | DSLSLSPGK | |
| TY25531 | EAVVTQEPSLTVSPGGTVTLTCASSTGAVTTSNYPNWVQQKPGQAPRGLIGGT | 682 |
| | KFRAPGVPARFSGSLLGGKAALTLSGAQPEDEAEYYCALWYSNHWVFGGGTKL | |
| | TVLGGGGSGGGGSGGGGSGGGGSEVQLVESGGGLVQPGGSLRLSCAASGFTFS | |
| | SYAIHWVRQAPGKGLEWVGRIRSKYNNYATYYAESVKDRFTISRDDSKNTLYL | |
| | QLNSLRAEDTAVYYCARHGNFGNSYVSYWAYWGQGTLVTVSSEPKSSDKTHTC | |
| | PPCPAPELLGGPSVFLFPPKPKDTLMISRTPEVTCVVVDVSHEDPEVKFNWYV | |
| | DGVEVHNAKTKPREEQYASTYRVVSVLTVLHQDWLNGKEYKCKVSNKALPAPI | |
| | EKTISKAKGQPREPQVYTDPPSRDELTKNQVSLTCLVDGFYPSDIAVEWESNG | |
| | QPENCYKTTPPVLDSDGSFFLYSKLTVDKSRWQQGNVFSCSVMHEALHNHYTQ | |
| | DSLSLSPGK | |

### Assessment of SP34 Variants

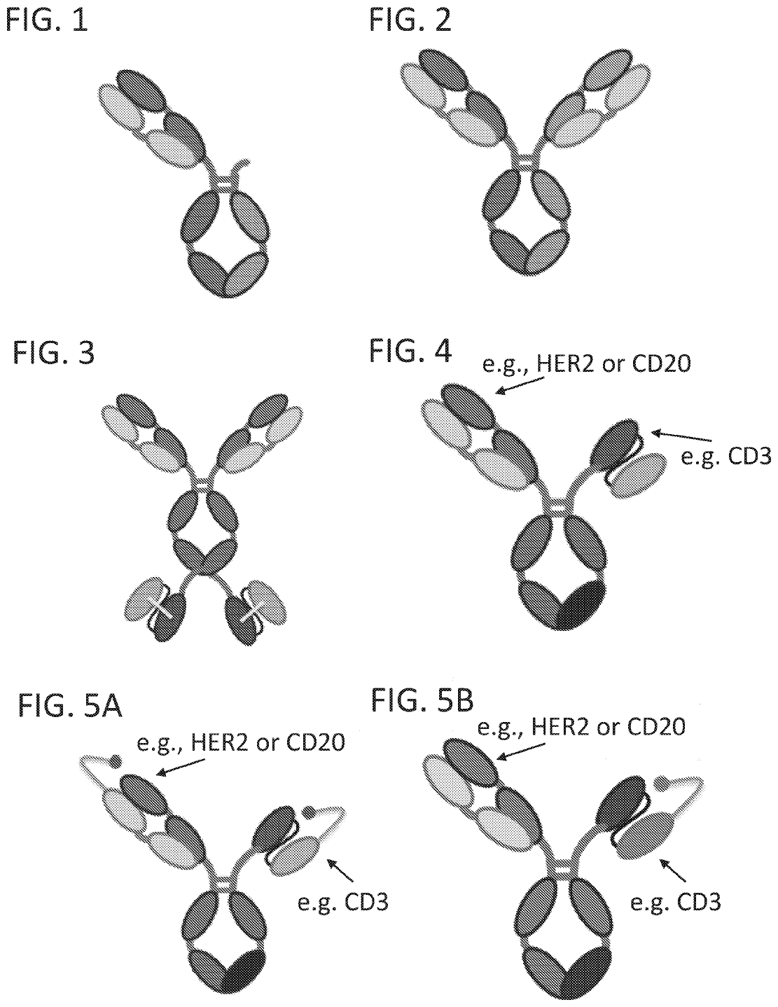

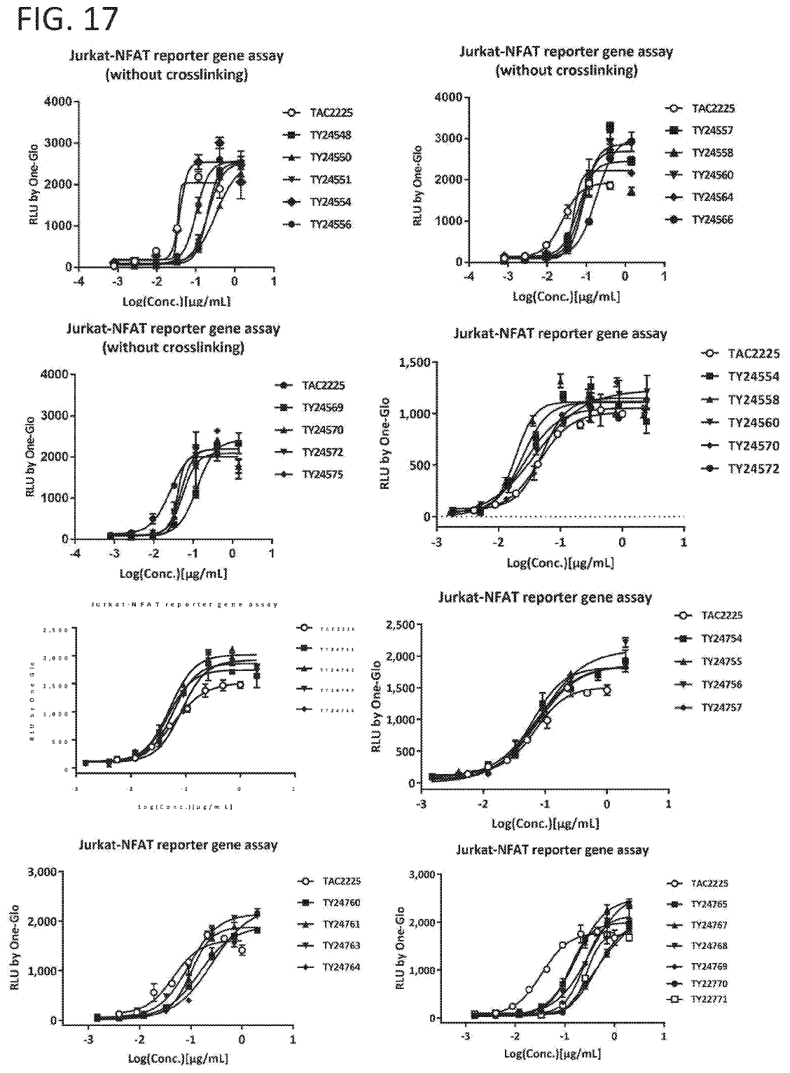

The activity of the antibodies was measured in a Jurkat-NFAT reporter assay (FIG. 17). Jurkat-NFAT-luc2 cells were thawed, washed and cultured in the incubator. Sample dilutions of test articles were prepared in assay medium consisting of RPM 1640 supplemented with 1% FBS as indicated in the figure. 1×10⁵ Jurkat-NFAT-luc2 cells/well were added to the 96-well plate and incubated with 50 μL of test antibodies for 6 hours. After incubation of about 6 hours, 80 μL per well ONE-Glo Luciferase Assay buffer was added to the 96-well plate, and 100 μL supernatant was collected for measuring luminescence using a plate reader.

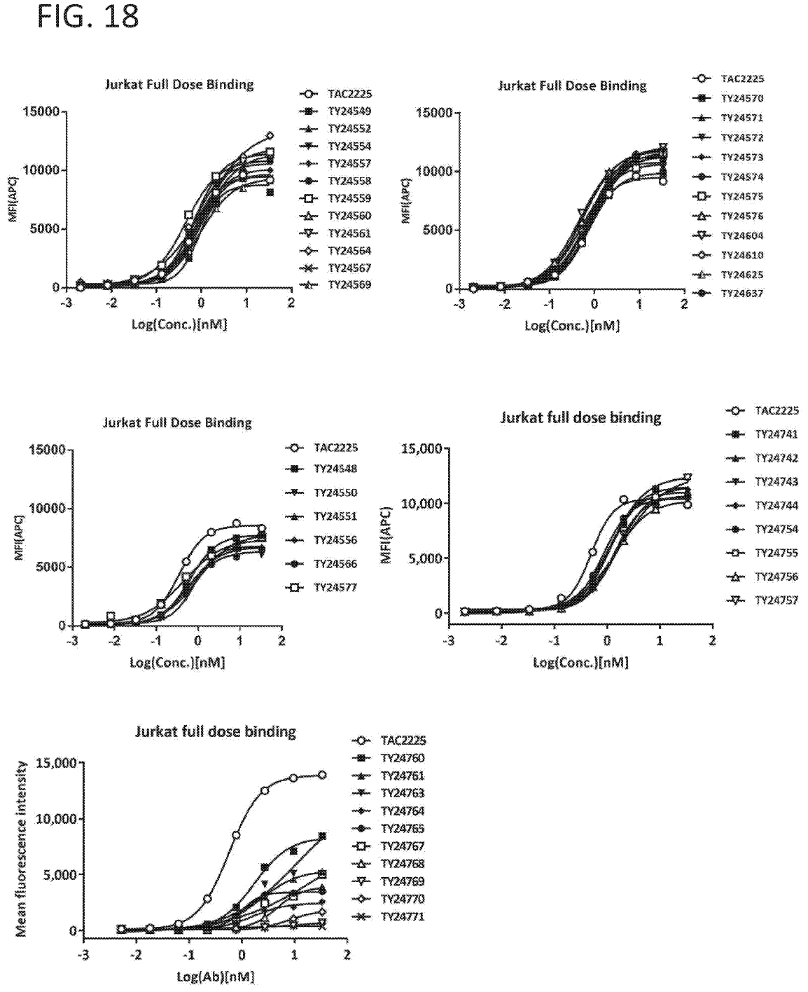

Dose-dependent binding of the antibodies to Jurkat cells was also measured (FIG. 18). The Jurkat cells were thawed, washed, and cultured in an incubator. Diluted samples of antibody were prepared in assay buffer consisting of DPBS supplemented with 2% FBS as indicated in the figure. Jurkat cells were washed and 1×10⁵ Jurkat cells/well were aliquoted into 96-well plate and incubated with test articles for 1 hour at 4° C. After centrifugation and washing, Jurkat cells were further incubated with the secondary antibody APC anti-Human IgG for 30 minutes at 4° C. After centrifugation and washing, the Jurkat cells labelled with antibodies were re-suspended in 2% FBS DPBS and then were analyzed by FACS.

### Results

As shown in FIG. 17, all tested antibodies activated the NFAT reporter with a potency comparable to control antibody TAC2225. As shown in FIG. 18, all tested antibodies showed levels of Jurkat cell binding activity that were similar to control antibody TAC2225. Antibodies TY24742, TY24557, TY24563, TY24566, TY24569, and TY24555 were chosen for further analysis.

The dissociation constants of further anti-CD3 antibodies derived from SP34 were determined using a Fortebio system. Further, the ability of the antibodies to measure CD3 in ELISAs and Jurkat cells was measured. As shown in Table 5I, anti-CD3 antibodies had a variety of binding affinities. In Table 5I, a dash indicates that data is not available for a given sample.

TABLE 5I
Anti-CD3 antibody CD3 binding data

| | Fortebio K_{D} | ELISA | Jurkat cell binding |
|---|---|---|---|
| IgG ID | (nM) | EC₅₀ (nM) | EC₅₀ (nM) |
| TY25520 | 8.03 | 45.1 | — |
| TY25521 | 13.1 | 56.6 | — |
| TY25523 | 17.2 | 65.8 | — |
| TY25524 | 8.04 | 555.6 | 260 |
| TY25525 | 3.65 | 2.66 | 6.88 |
| TY25526 | — | 53.6 | — |
| TY25527 | 28.3 | 362.7 | — |
| TY25528 | ND | 483600 | >1000 |
| TY25529 | 51.8 | 597.7 | — |
| TY25531 | 8.53 | 432.3 | — |

### Example 5. Generation and Characterization of Heterodimeric HER2xCD3 T-Cell-Engaging Bispecific Antibody

### Materials and Methods

### Generation of Anti-HER2 and Anti-CD3 Bispecific Antibodies Using Variants of Anti-CD3 Antibody SP34

A heterodimeric bispecific (anti-HER2 and anti-CD3) scaffold was designed using the TYM13 Fc mutant. Specifically, the anti-HER2 light chain-heavy chain half antibody trastuzumab and an anti-CD3 scFv-Fc chain were combined to form a bispecific antibody, with TYM13 mutations in heterodimeric-Fc domain. The SP34 variants TY24742, TY24557, TY24563, TY24566, TY24569, and TY24555 described in Example 4 were selected for constructing CD3 bispecific T-cell-engaging bispecific antibodies using this scaffold. In addition, variants of TY24742 and TY24557 were generated and constructed using the same format of CD3 bispecific antibody. For comparison, corresponding antibodies having “Xencor mutations” E357Q, S364K-L368′D, K370'S were also constructed (see summary in Table 6, below).

To generate the bispecific antibodies, plasmids encoding the heavy chain, light chain, and scFv-Fc chain were transiently transfected into mammalian cells. Bispecific antibody-containing cell culture supernatants were harvested 7 days after transfection by centrifugation at 14000 g for 30 minutes, and were filtered through a sterile filter (0.22 μm). Antibodies were purified by protein A affinity chromatography using MabSelect SuRe prepacked columns (GE Healthcare) and were subsequently buffer exchanged in 20 mM histidine (pH 5.5) buffer.

### Assessment of Anti-HER2 and Anti-CD3 Bispecific Antibodies

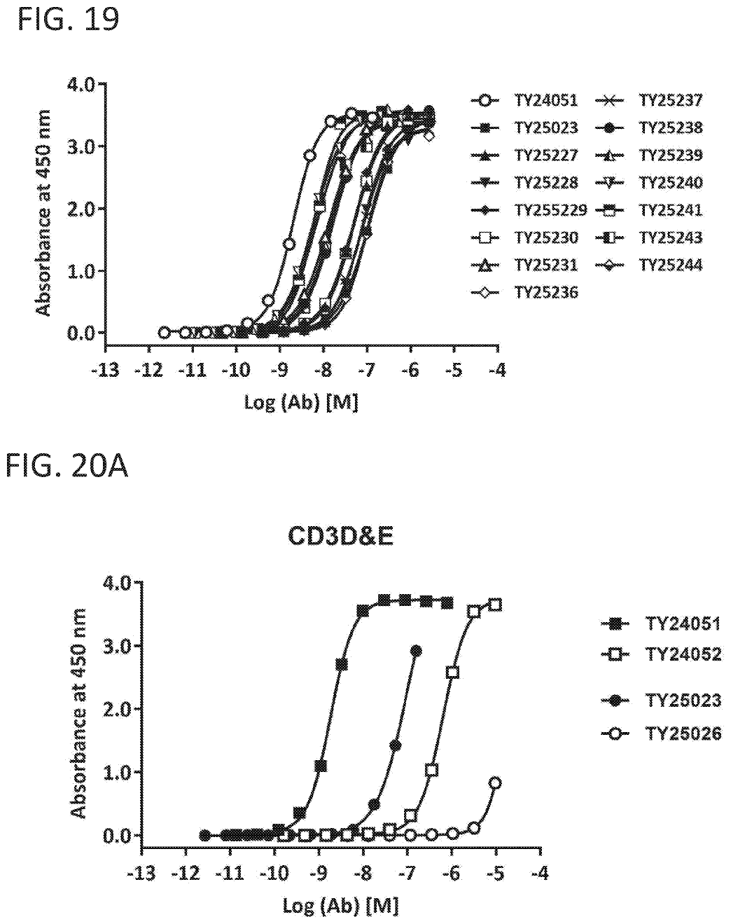

The binding affinities of the anti-CD3 variant bispecific antibodies were analyzed through enzyme-linked immunosorbent assays (ELISAs) (see FIG. 19). 2 μg/mL of human CD3 (ε and δ chain heterodimer) fused with human Fc fragment, were prepared and used to coat the ELISA plate at 2-8° C. overnight. After washing and blocking, 50 μL serial diluted IgGs were added and incubated at 37° C. for 1 hour. Plates were washed three times and then incubated with 50 μL/well TMB substrate at room temperature for about 20 minutes. Absorbance at 450 nm was measured after the reaction was stopped. The concentration of each antibody that produced half-maximal binding to CD36 is reported as the EC₅₀ in nM, in Table 6, below.

### Results

As shown in Table 6A and FIG. 19, the anti-CD3 and anti-HER2 antibodies variants showed a wide range of binding activities, with EC₅₀s ranging from 5.7 nM to 110.6 nM. Antibody TY25023 had the highest EC₅₀ value of 110.6 nM, indicating that, of the antibodies tested, TY25023 had the lowest affinity for binding CD3εδ. TY25236 and TY25023, which were both constructed using the an57-CD3 VH and VL sequences of TY24742 but were constructed in different bispecific formats, exhibited similar activity levels, binding CD3εδ at an EC₅₀ of 100.8 nM and 110.6 nM, respectively.

TABLE 6A
List of HER2 × CD3 bispecific antibodies
consisting of different anti-CD3 scFv mutants

| | | | | ELISA | SEC |
|---|---|---|---|---|---|
| | | anti-CD3 | Titer | EC₅₀ | HMW |
| IgG ID | Fc mutant | Variant | (mg/L) | (nM) | (%) |
| TY24051 | TYM13, N297A | TAC2225 | 64.7 | 2.0 | 3.1 |
| TY25236 | E357Q, S364K- | TY24742 | 22.8 | 100.8 | ND |
| | L368′D, K370′S | | | | |
| TY25023 | TYM13, N297A | TY24742 | 17.7 | 110.6 | 3.1 |
| TY25024 | TYM13, N297A | TY24757 | 12.1 | NA | 2.4 |
| TY25237 | TYM13, N297A | TY24742- | 13.7 | 79.3 | 3.1 |
| | | Mut1 | | | |
| TY25228 | TYM13, N297A | TY24563 | 32.9 | 76.6 | 2 |
| TY25227 | TYM13, N297A | TY24557 | 34.4 | 51.8 | 2.4 |
| TY25230 | TYM13, N297A | TY24569 | 43.2 | 50.9 | 2.8 |
| TY25229 | TYM13, N297A | TY24566 | 43.1 | 50.1 | 2.7 |
| TY25238 | TYM13, N297A | TY24742- | 32.9 | 16.9 | 2.3 |
| | | Mut2 | | | |
| TY25239 | TYM13, N297A | TY24742- | 32 | 15.6 | 2.9 |
| | | Mut3 | | | |
| TY25243 | TYM13, N297A | TY24757- | 31.5 | 14.1 | 2.2 |
| | | Mut2 | | | |
| TY25231 | TYM13, N297A | TY24555 | 36.5 | 12.9 | 2.6 |
| TY25244 | TYM13, N297A | TY24757- | 30.5 | 7.1 | 3.7 |
| | | Mut3 | | | |
| TY25241 | TYM13, N297A | TY24742- | 30.6 | 6.2 | 1.9 |
| | | Mut5 | | | |
| TY25240 | TYM13, N297A | TY24742- | 34.7 | 5.7 | 2.6 |
| | | Mut4 | | | |

The ability of HER2xCD3 bispecific antibodies to bind CD3 was measured using Biacore SPR and Fortebio assays, as shown in Table 6B. In Table 6B, a dash indicates that data is not available for a given sample.

TABLE 6B
HER2 × CD3 bispecific antibody CD3 binding data

| | anti-CD3 | Biacore | Cy Biacore | Fortebio |
|---|---|---|---|---|
| IgG ID | Variant | K_{D} (nM) | K_{D} (nM) | K_{D} (nM) |
| TY24051 | TAC2225 | 2.55 | 3.81 | 6.64 |
| TY25023 | TY24742 | 51.47 | 104.4 | 45.38 |
| TY25227 | TY24557 | 1.87 | — | 38.2 |
| TY25238 | TY24742-Mut2 | 3.33 | — | 16.63 |
| TY25243 | TY24757-Mut2 | — | — | 18.8 |
| TY25231 | TY24555 | — | — | 19 |
| TY25244 | TY24757-Mut3 | — | — | 11.5 |
| TY25241 | TY24742-Mut5 | — | — | 9.87 |
| TY25240 | TY24742-Mut4 | — | — | 10.7 |

Further experiments were performed to compare the CD3 binding, Jurkat cell binding, cell killing activity, and reporter activation of the bispecific antibodies TY24051 and TY25023, as shown in Table 6C. In Table 6C, a dash indicates that data is not available for a given sample.

TABLE 6C
TY24051 and TY25023 CD3 binding and activity data

| | | cy | | | Jurkat cell | | | | | Reporter assay | | |
|---|---|---|---|---|---|---|---|---|---|---|---|---|
| | | ELISA | ELISA | | binding | | Cell killing | | | Jurkat-NFAT | | |
| | anti-CD3 | EC₅₀ | EC₅₀ | | EC₅₀ | | assay EC₅₀ | | | reporter assay EC₅₀ | | |
| IgG ID | Variant | (nM) | (nM) | | (nM) | | (ng/mL) | | | (ng/mL) | | |
| TY24051 | TAC2225 | 0.9 | 1 | 8.05 | 15.3 | 10.2 | 0.06 | 0.103 | 0.17 | 1.22 | 1.11 | 0.74 |
| TY25023 | TY24742 | 22.5 | 43.4 | — | >1000 | 480 | 0.05 | 0.243 | 0.472 | 4.14 | 0.91 | 1.75 |

The amino acid sequences of the CDRs of the anti-CD3 scFv of HER2xCD3 bispecific antibodies are shown in Table 7, below. In Table 7, the CDRs are defined according to the Kabat numbering scheme.

TABLE 7
Amino acid sequences of anti-CD3 CDRs

| IgG ID | | | | |
|---|---|---|---|---|
| (bispecific | IgG ID | | | SEQ ID |
| antibody) | (CD3 scFv) | CDR | Amino Acid Sequence | NO |
| TY24051 | TAC2225 | CDR-H1 | TYAMN | 376 |
| | | CDR-H2 | RIRSKYNNYATYYADSVKG | 391 |
| | | CDR-H3 | HGNFGNSYVSWFAY | 378 |
| | | CDR-L1 | GSSTGAVTTSNYAN | 396 |
| | | CDR-L2 | GTNKRAP | 380 |
| | | CDR-L3 | ALWYSNLWV | 381 |
| TY25236 | TY24742 | CDR-H1 | TYAIN | 390 |
| | (VL-02/VH- | CDR-H2 | RIRSKYNNYATYYAESVKD | 392 |
| | 28) | CDR-H3 | HGNFGTSYVSWFAY | 395 |
| | | CDR-L1 | GSSTGAVTTSNYPN | 397 |
| | | CDR-L2 | GTNKRAP | 380 |
| | | CDR-L3 | ALWYSDLWV | 400 |
| TY25023 | TY24742 | CDR-H1 | TYAIN | 390 |
| | (VL-02/VH- | CDR-H2 | RIRSKYNNYATYYAESVKD | 392 |
| | 28) | CDR-H3 | HGNFGTSYVSWFAY | 395 |
| | | CDR-L1 | GSSTGAVTTSNYPN | 397 |
| | | CDR-L2 | GTNKRAP | 380 |
| | | CDR-L3 | ALWYSDLWV | 400 |
| TY25024 | TY24757 | CDR-H1 | TYAIN | 390 |
| | (VL-03/VH- | CDR-H2 | RIRSKYNNYATYYAESVKD | 392 |
| | 28) | CDR-H3 | HGNFGTSYVSWFAY | 395 |
| | | CDR-L1 | GSSTGAVTTSNYAN | 396 |
| | | CDR-L2 | GTNKRAP | 380 |
| | | CDR-L3 | ALWYSTLWV | 401 |
| TY25237 | TY24742- | CDR-H1 | TYAIN | 390 |
| | Mut1 | CDR-H2 | RIRSKYNNYATYYADSVKD | 393 |
| | (VL-31/VH- | CDR-H3 | HGNFGTSYVSWFAY | 395 |
| | 35) | CDR-L1 | GSSTGAVTTSNYPN | 397 |
| | | CDR-L2 | GTNKRAP | 380 |
| | | CDR-L3 | ALWYSNLWV | 381 |
| TY25228 | TY24563 | CDR-H1 | TYAMN | 376 |
| | (VL-03/VH- | CDR-H2 | RIRSKYNNYATYYADSVKD | 393 |
| | 04) | CDR-H3 | HGNFGTSYVSWFAY | 395 |
| | | CDR-L1 | GSSTGAVTTSNYAN | 396 |
| | | CDR-L2 | GTNKRAP | 380 |
| | | CDR-L3 | ALWYSTLWV | 401 |
| TY25227 | TY24557 | CDR-H1 | TYAMN | 376 |
| | (VL-02/VH- | CDR-H2 | RIRSKYNNYATYYADSVKD | 393 |
| | 04) | CDR-H3 | HGNFGTSYVSWFAY | 395 |
| | | CDR-L1 | GSSTGAVTTSNYPN | 397 |
| | | CDR-L2 | GTNKRAP | 380 |
| | | CDR-L3 | ALWYSDLWV | 400 |
| TY25230 | TY24569 | CDR-H1 | TYAMN | 376 |
| | (VL-04/VH- | CDR-H2 | RIRSKYNNYATYYADSVKD | 393 |
| | 04) | CDR-H3 | HGNFGTSYVSWFAY | 395 |
| | | CDR-L1 | RSSTGAVTTSNYAN | 398 |
| | | CDR-L2 | GTKFRAP | 399 |
| | | CDR-L3 | ALWYSDLWV | 400 |
| TY25229 | TY24566 | CDR-H1 | TYAMN | 376 |
| | (VL-04/VH- | CDR-H2 | RIRSKYNNYATYYADSVKD | 393 |
| | 01) | CDR-H3 | HGNFGTSYVSWFAY | 395 |
| | | CDR-L1 | RSSTGAVTTSNYAN | 398 |
| | | CDR-L2 | GTKFRAP | 399 |
| | | CDR-L3 | ALWYSDLWV | 400 |
| TY25238 | TY24742- | CDR-H1 | TYAIN | 390 |
| | Mut2 | CDR-H2 | RIRSKYNNYATYYAESVKG | 394 |
| | (VL-16/VH- | CDR-H3 | HGNFGTSYVSWFAY | 395 |
| | 36) | CDR-L1 | GSSTGAVTTSNYPN | 397 |
| | | CDR-L2 | GTNKRAP | 380 |
| | | CDR-L3 | ALWYSNLWV | 381 |
| TY25239 | TY24742- | CDR-H1 | TYAIN | 390 |
| | Mut3 | CDR-H2 | RIRSKYNNYATYYADSVKG | 391 |
| | (VL-32/VH- | CDR-H3 | HGNFGTSYVSWFAY | 395 |
| | 37) | CDR-L1 | GSSTGAVTTSNYAN | 396 |
| | | CDR-L2 | GTNKRAP | 380 |
| | | CDR-L3 | ALWYSNLWV | 381 |
| TY25243 | TY24757- | CDR-H1 | TYAIN | 390 |
| | Mut2 | CDR-H2 | RIRSKYNNYATYYAESVKG | 394 |
| | (VL-32/VH- | CDR-H3 | HGNFGTSYVSWFAY | 395 |
| | 36) | CDR-L1 | GSSTGAVTTSNYAN | 396 |
| | | CDR-L2 | GTNKRAP | 380 |
| | | CDR-L3 | ALWYSNLWV | 381 |
| TY25231 | TY24555 | CDR-H1 | TYAMN | 376 |
| | (VL-02/VH- | CDR-H2 | RIRSKYNNYATYYADSVKG | 391 |
| | 02) | CDR-H3 | HGNFGNSYVSWFAY | 378 |
| | | CDR-L1 | GSSTGAVTTSNYPN | 397 |
| | | CDR-L2 | GTNKRAP | 380 |
| | | CDR-L3 | ALWYSDLWV | 400 |
| TY25244 | TY24757- | CDR-H1 | TYAIN | 390 |
| | Mut3 | CDR-H2 | RIRSKYNNYATYYADSVKD | 393 |
| | (VL-32/VH- | CDR-H3 | HGNFGNSYVSWFAY | 378 |
| | 39) | CDR-L1 | GSSTGAVTTSNYAN | 396 |
| | | CDR-L2 | GTNKRAP | 380 |
| | | CDR-L3 | ALWYSNLWV | 381 |
| TY25241 | TY24742- | CDR-H1 | TYAIN | 390 |
| | Mut5 | CDR-H2 | RIRSKYNNYATYYADSVKG | 391 |
| | (VL-32/VH- | CDR-H3 | HGNFGNSYVSWFAY | 378 |
| | 38) | CDR-L1 | GSSTGAVTTSNYAN | 396 |
| | | CDR-L2 | GTNKRAP | 380 |
| | | CDR-L3 | ALWYSNLWV | 381 |
| TY25240 | TY24742- | CDR-H1 | TYAIN | 390 |
| | Mut4 | CDR-H2 | RIRSKYNNYATYYADSVKG | 391 |
| | (VL-16/VH- | CDR-H3 | HGNFGNSYVSWFAY | 378 |
| | 38) | CDR-L1 | GSSTGAVTTSNYPN | 397 |
| | | CDR-L2 | GTNKRAP | 380 |
| | | CDR-L3 | ALWYSNLWV | 381 |

The amino acid sequences of the VH and VL regions of the anti-CD3 scFv of HER2xCD3 bispecific antibodies are provided in Table 8, below.

TABLE 8
Amino acid sequences of anti-CD3 VHs and VLs

| | Region of | | SEQ ID |
|---|---|---|---|
| IgG ID | antibody | Amino Acid Sequence | NO |
| TY24051 | anti-CD3 | EVQLVESGGGLVQPGGSLRLSCAASGFTFNTYAMNWVRQAPGKGLEW | 67 |
| | VH | VGRIRSKYNNYATYYADSVKGRFTISRDDSKNTLYLQMNSLRAEDTA | |
| | | VYYCVRHGNFGNSYVSWFAYWGQGTLVTVSS | |
| TY24051 | anti-CD3 | QAVVTQEPSLTVSPGGTVTLTCGSSTGAVTTSNYANWVQQKPGQAPR | 68 |
| | VL | GLIGGTNKRAPGVPARFSGSLLGGKAALTLSGAQPEDEAEYYCALWY | |
| | | SNLWVFGGGTKLTVL | |
| TY25236 | anti-CD3 | EVQLVESGGGLVQPGGSLRLSCAASGFTFSTYAINWVRQAPGKGLEW | 402 |
| | VH | VSRIRSKYNNYATYYAESVKDRFTISRDDSKNTLYLQINSLRAEDTA | |
| | | VYYCVRHGNFGTSYVSWFAYWGQGTLVTVSS | |
| TY25236 | anti-CD3 | EAVVTQEPSLTVSPGGTVTLTCGSSTGAVTTSNYPNWVQQKPGQAPR | 403 |
| | VL | GLIGGTNKRAPGTPARFSGSLLGGKAALTLSGAQPEDEAEYYCALWY | |
| | | SDLWVFGGGTKLTVL | |
| TY25023 | anti-CD3 | EVQLVESGGGLVQPGGSLRLSCAASGFTFSTYAINWVRQAPGKGLEW | 402 |
| | VH | VSRIRSKYNNYATYYAESVKDRFTISRDDSKNTLYLQINSLRAEDTA | |
| | | VYYCVRHGNFGTSYVSWFAYWGQGTLVTVSS | |
| TY25023 | anti-CD3 | EAVVTQEPSLTVSPGGTVTLTCGSSTGAVTTSNYPNWVQQKPGQAPR | 403 |
| | VL | GLIGGTNKRAPGTPARFSGSLLGGKAALTLSGAQPEDEAEYYCALWY | |
| | | SDLWVFGGGTKLTVL | |
| TY25024 | anti-CD3 | EVQLVESGGGLVQPGGSLRLSCAASGFTFSTYAINWVRQAPGKGLEW | 402 |
| | VH | VSRIRSKYNNYATYYAESVKDRFTISRDDSKNTLYLQINSLRAEDTA | |
| | | VYYCVRHGNFGTSYVSWFAYWGQGTLVTVSS | |
| TY25024 | anti-CD3 | QAVVTQEPSLTVSPGGTVTLTCGSSTGAVTTSNYANWVQQKPGQAPR | 404 |
| | VL | GLIGGTNKRAPGTPARFSGSLLGGKAALTLSGAQPEDEAEYYCALWY | |
| | | STLWVFGGGTKLTVL | |
| TY25237 | anti-CD3 | EVQLVESGGGLVQPGGSLRLSCAASGFTFNTYAINWVRQAPGKGLEW | 405 |
| | VH | VSRIRSKYNNYATYYADSVKDRFTISRDDSKNTLYLQINSLRAEDTA | |
| | | VYYCVRHGNFGTSYVSWFAYWGQGTLVTVSS | |
| TY25237 | anti-CD3 | QAVVTQEPSLTVSPGGTVTLTCGSSTGAVTTSNYPNWVQQKPGQAPR | 406 |
| | VL | GLIGGTNKRAPGTPARFSGSLLGGKAALTLSGAQPEDEAEYYCALWY | |
| | | SNLWVFGGGTKLTVL | |
| TY25228 | anti-CD3 | EVQLVESGGGLVKPGGSLRLSCAASGFTFSTYAMNWVRQAPGKGLEW | 407 |
| | VH | VSRIRSKYNNYATYYADSVKDRFTISRDDSKNTLYLQINSLRAEDTA | |
| | | VYYCVRHGNFGTSYVSWFAYWGQGTLVTVSS | |
| TY25228 | anti-CD3 | QAVVTQEPSLTVSPGGTVTLTCGSSTGAVTTSNYANWVQQKPGQAPR | 404 |
| | VL | GLIGGTNKRAPGTPARFSGSLLGGKAALTLSGAQPEDEAEYYCALWY | |
| | | STLWVFGGGTKLTVL | |
| TY25227 | anti-CD3 | EVQLVESGGGLVKPGGSLRLSCAASGFTFSTYAMNWVRQAPGKGLEW | 407 |
| | VH | VSRIRSKYNNYATYYADSVKDRFTISRDDSKNTLYLQINSLRAEDTA | |
| | | VYYCVRHGNFGTSYVSWFAYWGQGTLVTVSS | |
| TY25227 | anti-CD3 | EAVVTQEPSLTVSPGGTVTLTCGSSTGAVTTSNYPNWVQQKPGQAPR | 403 |
| | VL | GLIGGTNKRAPGTPARFSGSLLGGKAALTLSGAQPEDEAEYYCALWY | |
| | | SDLWVFGGGTKLTVL | |
| TY25230 | anti-CD3 | EVQLVESGGGLVKPGGSLRLSCAASGFTFSTYAMNWVRQAPGKGLEW | 407 |
| | VH | VSRIRSKYNNYATYYADSVKDRFTISRDDSKNTLYLQINSLRAEDTA | |
| | | VYYCVRHGNFGTSYVSWFAYWGQGTLVTVSS | |
| TY25230 | anti-CD3 | EAVVTQEPSLTVSPGGTVTLTCRSSTGAVTTSNYANWVQQKPGQAPR | 408 |
| | VL | GLIGGTKFRAPGVPARFSGSLLGGKAALTLSGAQPEDEAEYYCALWY | |
| | | SDLWVFGGGTKLTVL | |
| TY25229 | anti-CD3 | EVQLVESGGGLVQPGGSLRLSCAASGFTFSTYAMNWVRQAPGKGLEW | 409 |
| | VH | VSRIRSKYNNYATYYADSVKDRFTISRDDSKNTLYLQINSLRAEDTA | |
| | | VYYCVRHGNFGTSYVSWFAYWGQGTLVTVSS | |
| TY25229 | anti-CD3 | EAVVTQEPSLTVSPGGTVTLTCRSSTGAVTTSNYANWVQQKPGQAPR | 408 |
| | VL | GLIGGTKFRAPGVPARFSGSLLGGKAALTLSGAQPEDEAEYYCALWY | |
| | | SDLWVFGGGTKLTVL | |
| TY25238 | anti-CD3 | EVQLVESGGGLVQPGGSLRLSCAASGFTFNTYAINWVRQAPGKGLEW | 410 |
| | VH | VGRIRSKYNNYATYYAESVKGRFTISRDDSKNTLYLQINSLRAEDTA | |
| | | VYYCVRHGNFGTSYVSWFAYWGQGTLVTVSS | |
| TY25238 | anti-CD3 | QAVVTQEPSLTVSPGGTVTLTCGSSTGAVTTSNYPNWVQQKPGQAPR | 411 |
| | VL | GLIGGTNKRAPGVPARFSGSLLGGKAALTLSGAQPEDEAEYYCALWY | |
| | | SNLWVFGGGTKLTVL | |
| TY25239 | anti-CD3 | EVQLVESGGGLVQPGGSLRLSCAASGFTFNTYAINWVRQAPGKGLEW | 412 |
| | VH | VGRIRSKYNNYATYYADSVKGRFTISRDDSKNTLYLQINSLRAEDTA | |
| | | VYYCVRHGNFGTSYVSWFAYWGQGTLVTVSS | |
| TY25239 | anti-CD3 | QAVVTQEPSLTVSPGGTVTLTCGSSTGAVTTSNYANWVQQKPGQAPR | 413 |
| | VL | GLIGGTNKRAPGTPARFSGSLLGGKAALTLSGAQPEDEAEYYCALWY | |
| | | SNLWVFGGGTKLTVL | |
| TY25243 | anti-CD3 | EVQLVESGGGLVQPGGSLRLSCAASGFTFNTYAINWVRQAPGKGLEW | 410 |
| | VH | VGRIRSKYNNYATYYAESVKGRFTISRDDSKNTLYLQINSLRAEDTA | |
| | | VYYCVRHGNFGTSYVSWFAYWGQGTLVTVSS | |
| TY25243 | anti-CD3 | QAVVTQEPSLTVSPGGTVTLTCGSSTGAVTTSNYANWVQQKPGQAPR | 413 |
| | VL | GLIGGTNKRAPGTPARFSGSLLGGKAALTLSGAQPEDEAEYYCALWY | |
| | | SNLWVFGGGTKLTVL | |
| TY25231 | anti-CD3 | EVQLVESGGGLVQPGGSLRLSCAASGFTFNTYAMNWVRQAPGKGLEW | 414 |
| | VH | VSRIRSKYNNYATYYADSVKGRFTISRDDSKNTLYLQLNSLRAEDTA | |
| | | VYYCVRHGNFGNSYVSWFAYWGQGTLVTVSS | |
| TY25231 | anti-CD3 | EAVVTQEPSLTVSPGGTVTLTCGSSTGAVTTSNYPNWVQQKPGQAPR | 403 |
| | VL | GLIGGTNKRAPGTPARFSGSLLGGKAALTLSGAQPEDEAEYYCALWY | |
| | | SDLWVFGGGTKLTVL | |
| TY25244 | anti-CD3 | EVQLVESGGGLVQPGGSLRLSCAASGFTFNTYAINWVRQAPGKGLEW | 415 |
| | VH | VGRIRSKYNNYATYYADSVKDRFTISRDDSKNTLYLQINSLRAEDTA | |
| | | VYYCVRHGNFGNSYVSWFAYWGQGTLVTVSS | |
| TY25244 | anti-CD3 | QAVVTQEPSLTVSPGGTVTLTCGSSTGAVTTSNYANWVQQKPGQAPR | 413 |
| | VL | GLIGGTNKRAPGTPARFSGSLLGGKAALTLSGAQPEDEAEYYCALWY | |
| | | SNLWVFGGGTKLTVL | |
| TY25241 | anti-CD3 | EVQLVESGGGLVQPGGSLRLSCAASGFTFNTYAINWVRQAPGKGLEW | 416 |
| | VH | VGRIRSKYNNYATYYADSVKGRFTISRDDSKNTLYLQINSLRAEDTA | |
| | | VYYCVRHGNFGNSYVSWFAYWGQGTLVTVSS | |
| TY25241 | anti-CD3 | QAVVTQEPSLTVSPGGTVTLTCGSSTGAVTTSNYANWVQQKPGQAPR | 413 |
| | VL | GLIGGTNKRAPGTPARFSGSLLGGKAALTLSGAQPEDEAEYYCALWY | |
| | | SNLWVFGGGTKLTVL | |
| TY25240 | anti-CD3 | EVQLVESGGGLVQPGGSLRLSCAASGFTFNTYAINWVRQAPGKGLEW | 416 |
| | VH | VGRIRSKYNNYATYYADSVKGRFTISRDDSKNTLYLQINSLRAEDTA | |
| | | VYYCVRHGNFGNSYVSWFAYWGQGTLVTVSS | |
| TY25240 | anti-CD3 | QAVVTQEPSLTVSPGGTVTLTCGSSTGAVTTSNYPNWVQQKPGQAPR | 411 |
| | VL | GLIGGTNKRAPGVPARFSGSLLGGKAALTLSGAQPEDEAEYYCALWY | |
| | | SNLWVFGGGTKLTVL | |

The amino acid sequences of the anti-CD3 scFv of TY25023 and TY25238 are provided in Table 9, below.

TABLE 9
Amino acid sequences of TY25023 and TY25238 anti-CD3 VH scFvs

| | Region of | | SEQ ID |
|---|---|---|---|
| IgG ID | antibody | Amino Acid Sequence | NO |
| TY25023 | anti-CD3 | EAVVTQEPSLTVSPGGTVTLTCGSSTGAVTTSNYPNWVQQKPGQ | 421 |
| | scFv | APRGLIGGTNKRAPGTPARFSGSLLGGKAALTLSGAQPEDEAEY | |
| | | YCALWYSDLWVFGGGTKLTVLGGGGSGGGGSGGGGSGGGGSEVQ | |
| | | LVESGGGLVQPGGSLRLSCAASGFTFSTYAINWVRQAPGKGLEW | |
| | | VSRIRSKYNNYATYYAESVKDRFTISRDDSKNTLYLQINSLRAE | |
| | | DTAVYYCVRHGNFGTSYVSWFAYWGQGTLVTVSS | |
| TY25238 | anti-CD3 | QAVVTQEPSLTVSPGGTVTLTCGSSTGAVTTSNYPNWVQQKPGQ | 422 |
| | scFv | APRGLIGGTNKRAPGVPARFSGSLLGGKAALTLSGAQPEDEAEY | |
| | | YCALWYSNLWVFGGGTKLTVLGGGGSGGGGSGGGGSGGGGSEVQ | |
| | | LVESGGGLVQPGGSLRLSCAASGFTFNTYAINWVRQAPGKGLEW | |
| | | VGRIRSKYNNYATYYAESVKGRFTISRDDSKNTLYLQINSLRAE | |
| | | DTAVYYCVRHGNFGTSYVSWFAYWGQGTLVTVSS | |

The anti-HER2 arms of TY25023 and TY25238 are identical to the parent anti-HER2 antibody trastuzumab. The anti-HER2 CDRs of TY25023 and TY25238 are provided in Table 10, below. In Table 10, the CDRs are defined according to the Kabat numbering scheme.

TABLE 10
Amino acid sequences of TY25023 and TY25238
anti-HER2 CDRs

| CDR | TY25023 & TY25238 | SEQ ID NO |
|---|---|---|
| CDR-H1 | DTYIH | 423 |
| CDR-H2 | RIYPTNGYTRYADSVKG | 424 |
| CDR-H3 | WGGDGFYAMDY | 71 |
| CDR-L1 | RASQDVNTAVA | 72 |
| CDR-L2 | SASFLYS | 73 |
| CDR-L3 | QQHYTTPPT | 74 |

The amino acid sequences of the VH and VL regions of the anti-HER2 arms of TY25023 and TY25238 are provided in Table 11, below.

TABLE 11
Amino acid sequences of TY25023 and TY25238 anti-CD3 VH and VL

| | Region of | | SEQ ID |
|---|---|---|---|
| IgG ID | antibody | Amino Acid Sequence | NO |
| TY25023 | anti-HER2 | EVQLVESGGGLVQPGGSLRLSCAASGFNIKDTYIHWVRQAPGKG | 75 |
| and | VH | LEWVARIYPTNGYTRYADSVKGRFTISADTSKNTAYLQMNSLRA | |
| TY25238 | | EDTAVYYCSRWGGDGFYAMDYWGQGTLVTVSS | |
| TY25023 | anti-HER2 | DIQMTQSPSSLSASVGDRVTITCRASQDVNTAVAWYQQKPGKAP | 76 |
| and | VL | KLLIYSASFLYSGVPSRFSGSRSGTDFTLTISSLQPEDFATYYC | |
| TY25238 | | QQHYTTPPTFGQGTKVEIKR | |

Finally, the amino acid sequences of the full-length heavy and light chains of TY25023 and TY25238 are provided in Table 12, below. Heavy chain 1 (HC1) and light chain (LC1) are the anti-HER2 portions of the antibodies, and heavy chain 2 (HC2) is the anti-CD3 portion of the antibody. The heavy chains sequences below are provided with the C-terminal lysine.

TABLE 12
Amino acid sequences of TY25023 and TY25238 heavy and light chains

| | Antibody | | SEQ ID |
|---|---|---|---|
| IgG ID | chain | Amino Acid Sequence | NO |
| TY25023 | LC1 | DIQMTQSPSSLSASVGDRVTITCRASQDVNTAVAWYQQKPGKAP | 112 |
| | | KLLIYSASFLYSGVPSRFSGSRSGTDFTLTISSLQPEDFATYYC | |
| | | QQHYTTPPTFGQGTKVEIKRTVAAPSVFIFPPSDEQLKSGTASV | |
| | | VCLLNNFYPREAKVQWKVDNALQSGNSQESVTEQDSKDSTYSLS | |
| | | STLTLSKADYEKHKVYACEVTHQGLSSPVTKSFNRGEC | |
| TY25023 | HC1 | EVQLVESGGGLVQPGGSLRLSCAASGFNIKDTYIHWVRQAPGKG | 425 |
| | | LEWVARIYPTNGYTRYADSVKGRFTISADTSKNTAYLQMNSLRA | |
| | | EDTAVYYCSRWGGDGFYAMDYWGQGTLVTVSSASTKGPSVFPLA | |
| | | PSSKSTSGGTAALGCLVKDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPELLGGPSVFLFPPKPKDTLMISRTPEVTC | |
| | | VVVDVSHEDPEVKFNWYVDGVEVHNAKTKPREEQYASTYRVVSV | |
| | | LTVLHQDWLNGKEYKCKVSNKALPAPIEKTISKAKGQPREPQVY | |
| | | TLPPSRKKLTKNQVKLTCLVKGFYPSDIAVEWESNGQPENNYKT | |
| | | TPPVLDCDGSFFLYSKLTVDKSRWQQGNVFSCSVMHEALHNHYT | |
| | | QKSLSLSPGK | |
| TY25023 | HC2 | EAVVTQEPSLTVSPGGTVTLTCGSSTGAVTTSNYPNWVQQKPGQ | 426 |
| | | APRGLIGGTNKRAPGTPARFSGSLLGGKAALTLSGAQPEDEAEY | |
| | | YCALWYSDLWVFGGGTKLTVLGGGGSGGGGSGGGGSGGGGSEVQ | |
| | | LVESGGGLVQPGGSLRLSCAASGFTFSTYAINWVRQAPGKGLEW | |
| | | VSRIRSKYNNYATYYAESVKDRFTISRDDSKNTLYLQINSLRAE | |
| | | DTAVYYCVRHGNFGTSYVSWFAYWGQGTLVTVSSEPKSSDKTHT | |
| | | CPPCPAPELLGGPSVFLFPPKPKDTLMISRTPEVTCVVVDVSHE | |
| | | DPEVKFNWYVDGVEVHNAKTKPREEQYASTYRVVSVLTVLHQDW | |
| | | LNGKEYKCKVSNKALPAPIEKTISKAKGQPREPQVYTDPPSRDE | |
| | | LTKNQVSLTCLVDGFYPSDIAVEWESNGQPENCYKTTPPVLDSD | |
| | | GSFFLYSKLTVDKSRWQQGNVFSCSVMHEALHNHYTQDSLSLSP | |
| | | GK | |
| TY25238 | LC1 | DIQMTQSPSSLSASVGDRVTITCRASQDVNTAVAWYQQKPGKAP | 112 |
| | | KLLIYSASFLYSGVPSRFSGSRSGTDFTLTISSLQPEDFATYYC | |
| | | QQHYTTPPTFGQGTKVEIKRTVAAPSVFIFPPSDEQLKSGTASV | |
| | | VCLLNNFYPREAKVQWKVDNALQSGNSQESVTEQDSKDSTYSLS | |
| | | STLTLSKADYEKHKVYACEVTHQGLSSPVTKSFNRGEC | |
| TY25238 | HC1 | EVQLVESGGGLVQPGGSLRLSCAASGFNIKDTYIHWVRQAPGKG | 427 |
| | | LEWVARIYPTNGYTRYADSVKGRFTISADTSKNTAYLQMNSLRA | |
| | | EDTAVYYCSRWGGDGFYAMDYWGQGTLVTVSSASTKGPSVFPLA | |
| | | PSSKSTSGGTAALGCLVKDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPELLGGPSVFLFPPKPKDTLMISRTPEVTC | |
| | | VVVDVSHEDPEVKFNWYVDGVEVHNAKTKPREEQYASTYRVVSV | |
| | | LTVLHQDWLNGKEYKCKVSNKALPAPIEKTISKAKGQPREPQVY | |
| | | TLPPSRKKLTKNQVKLTCLVKGFYPSDIAVEWESNGQPENNYKT | |
| | | TPPVLDCDGSFFLYSKLTVDKSRWQQGNVFSCSVMHEALHNHYT | |
| | | QKSLSLSPGK | |
| TY25238 | HC2 | QAVVTQEPSLTVSPGGTVTLTCGSSTGAVTTSNYPNWVQQKPGQ | 428 |
| | | APRGLIGGTNKRAPGVPARFSGSLLGGKAALTLSGAQPEDEAEY | |
| | | YCALWYSNLWVFGGGTKLTVLGGGGSGGGGSGGGGSGGGGSEVQ | |
| | | LVESGGGLVQPGGSLRLSCAASGFTFNTYAINWVRQAPGKGLEW | |
| | | VGRIRSKYNNYATYYAESVKGRFTISRDDSKNTLYLQINSLRAE | |
| | | DTAVYYCVRHGNFGTSYVSWFAYWGQGTLVTVSSEPKSSDKTHT | |
| | | CPPCPAPELLGGPSVFLFPPKPKDTLMISRTPEVTCVVVDVSHE | |
| | | DPEVKFNWYVDGVEVHNAKTKPREEQYASTYRVVSVLTVLHQDW | |
| | | LNGKEYKCKVSNKALPAPIEKTISKAKGOPREPQVYTDPPSRDE | |
| | | LTKNQVSLTCLVDGFYPSDIAVEWESNGOPENCYKTTPPVLDSD | |
| | | GSFFLYSKLTVDKSRWQQGNVFSCSVMHEALHNHYTQDSLSLSP | |
| | | GK | |

### Example 6. Functional Characterization of Activatable Anti-CD3 Variant Bispecific Antibody

In order to explore the in vitro activity of the anti-CD3 bispecific antibody variants, the bispecific antibody with the weakest CD3-binding affinity (TY25023) was selected to generate a corresponding activatable bispecific antibody. TY24051 and its activatable bispecific antibody TY24052 (as described in Example 1) were also tested, as a comparison.

A. Generation of Activatable Bispecific Anti-CD3 and Anti-HER2 Antibody Derived from SP34 Variant

TY25026 was generated from TY25023 by the addition of a masking moiety and cleavable moiety (MM-CM) region on both the anti-CD3 and anti-HER2 arms. TY25362 was generated from TY25023 by the addition of a masking moiety and cleavage moiety (MM-CM) region on the anti-CD3 arm only (see schematic in FIG. 5B). The amino acid sequences of the MM-CM region for the anti-CD3 and anti-HER2 arrms of TY25026 are provided in Table 13A, below.

TABLE 13A
Amino acid sequences of TY25026 and TY25362 masking moieties and cleavage
moieties

| | | MM | | CM | |
|---|---|---|---|---|---|
| | | | SEQ | | SEQ |
| | | | ID | | ID |
| IgG ID | Arm | Amino Acid Sequence | NO | Amino Acid Sequence | NO |
| TY25026 | anti-CD3 | EVGSYPYDDPDCPSHESDCDQ | 417 | GGGPLGLAGGS | 418 |
| | arm | | | | |
| | anti- | ESDACDADPFDCQA | 419 | PLGLAGSGGS | 420 |
| | HER2 | | | | |
| | arm | | | | |
| TY25362 | anti-CD3 | EVGSYPYDDPDCPSHESDCDQ | 417 | GGGPLGLAGGS | 418 |
| | arm | | | | |

The amino acid sequences of the full-length heavy and light chains of TY25026 and TY25362 are provided in Table 13B, below. Heavy chain 1 (HC1) and light chain (LC1) are the anti-HER2 portions of the TY25026, and heavy chain 2 (HC2) is the anti-CD3 portion of TY25026. The heavy chains sequences below are provided with the C-terminal lysine residue included. Masking moiety sequences are bolded and underlined.

TABLE 13B
Amino acid sequences of TY25026 heavy and light chains

| | Region of | | SEQ ID |
|---|---|---|---|
| IgG ID | antibody | Amino Acid Sequence | NO |
| TY25026 | LC1 | ESDACDADPFDCQAPLGLAGSGGSDIQMTQSPSSLSASVGDRVT | 115 |
| | | ITCRASQDVNTAVAWYQQKPGKAPKLLIYSASFLYSGVPSRFSG | |
| | | SRSGTDFTLTISSLQPEDFATYYCQQHYTTPPTFGQGTKVEIKR | |
| | | TVAAPSVFIFPPSDEQLKSGTASVVCLLNNFYPREAKVQWKVDN | |
| | | ALQSGNSQESVTEQDSKDSTYSLSSTLTLSKADYEKHKVYACEV | |
| | | THQGLSSPVTKSFNRGEC | |
| TY25026 | HC1 | EVQLVESGGGLVQPGGSLRLSCAASGFNIKDTYIHWVRQAPGKG | 429 |
| | | LEWVARIYPTNGYTRYADSVKGRFTISADTSKNTAYLQMNSLRA | |
| | | EDTAVYYCSRWGGDGFYAMDYWGQGTLVTVSSASTKGPSVFPLA | |
| | | PSSKSTSGGTAALGCLVKDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPELLGGPSVFLFPPKPKDTLMISRTPEVTC | |
| | | VVVDVSHEDPEVKFNWYVDGVEVHNAKTKPREEQYASTYRVVSV | |
| | | LTVLHQDWLNGKEYKCKVSNKALPAPIEKTISKAKGQPREPQVY | |
| | | TLPPSRKKLTKNQVKLTCLVKGFYPSDIAVEWESNGQPENNYKT | |
| | | TPPVLDCDGSFFLYSKLTVDKSRWQQGNVFSCSVMHEALHNHYT | |
| | | QKSLSLSPGK | |
| TY25026 | HC2 | EVGSYPYDDPDCPSHESDCDQGGGPLGLAGGSEAVVTQEPSLTV | 430 |
| | | SPGGTVTLTCGSSTGAVTTSNYPNWVQQKPGQAPRGLIGGTNKR | |
| | | APGTPARFSGSLLGGKAALTLSGAQPEDEAEYYCALWYSDLWVF | |
| | | GGGTKLTVLGGGGSGGGGSGGGGSGGGGSEVQLVESGGGLVQPG | |
| | | GSLRLSCAASGFTFSTYAINWVRQAPGKGLEWVSRIRSKYNNYA | |
| | | TYYAESVKDRFTISRDDSKNTLYLQINSLRAEDTAVYYCVRHGN | |
| | | FGTSYVSWFAYWGQGTLVTVSSEPKSSDKTHTCPPCPAPELLGG | |
| | | PSVFLFPPKPKDTLMISRTPEVTCVVVDVSHEDPEVKFNWYVDG | |
| | | VEVHNAKTKPREEQYASTYRVVSVLTVLHQDWLNGKEYKCKVSN | |
| | | KALPAPIEKTISKAKGQPREPQVYTDPPSRDELTKNQVSLTCLV | |
| | | DGFYPSDIAVEWESNGQPENCYKTTPPVLDSDGSFFLYSKLTVD | |
| | | KSRWQQGNVFSCSVMHEALHNHYTQDSLSLSPGK | |

### B. Binding Assays

The binding affinities of the TY25023, TY25026, TY25041 and TY25042 were analyzed in an ELISA. 2 μg/mL of human CD3 (ε and δ chain heterodimer) fused with human Fc fragment was prepared and used to coat the ELISA plate at 2-8° C. overnight. After washing and blocking, 50 μL serial diluted IgGs were added and incubated at 37° C. for 1 hour. Plates were washed three times and then incubated with 50 μL/well TMB substrate at room temperature for about 20 minutes. Absorbance at 450 nm was measured after the reaction was stopped.

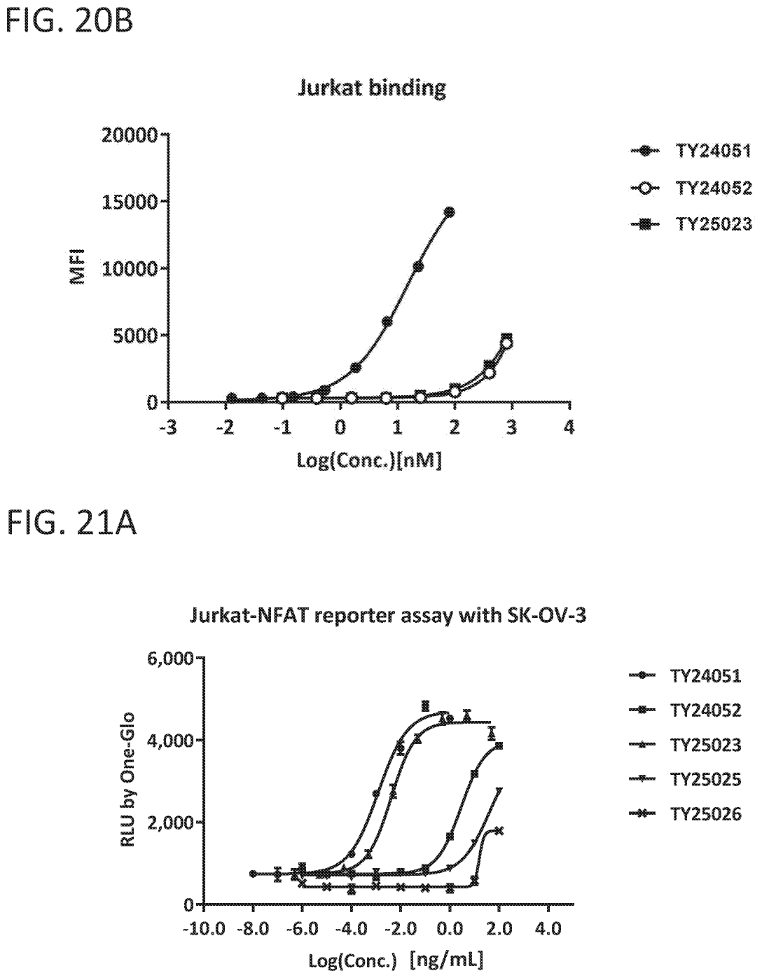

As shown in FIG. 20A, TY25023 exhibited weaker binding to CD3 compared to TY24051, and TY25026 exhibited weaker binding to CD3 TY24052. The activatable bispecific antibodies TY24052 and TY25026 showed reduced binding affinities compared to their parental bispecific antibodies. This result showed that the masking peptide blocked binding of the activatable antibodies to CD3.

To measure the binding of the antibodies to Jurkat cells, Jurkat cells were thawed, washed, and cultured in an incubator. Diluted samples of test antibodies were prepared in assay buffer consisting of DPBS supplemented with 2% FBS. Jurkat cells were washed and 1×10⁵ Jurkat cells/well were aliquoted into 96-well plate and incubated with test antibodies for 1 hour at 4° C. After centrifugation and washing, Jurkat cells were further incubated with a secondary, anti-Human IgG antibody (APC) for 30 minutes at 4° C. After centrifugation and washing, the Jurkat cells labelled with TY25023, TY25026, TY25041 and TY25042 were re-suspended in 2% FBS DPBS and then analyzed by FACS. As shown in FIG. 20B, TY25023 bound to Jurkat cells more weakly compared to TY24051, and bound at a similar level to TY24052.

### C. Jurkat-NFAT Reporter Assay

To measure antibody activity with a Jurkat-NFAT reporter assay, Jurkat-NFAT-luc2 cells were thawed, washed and cultured in the incubator. Sample dilutions of test articles were prepared in assay medium consisting of RPM 1640 supplemented with 1% FBS. 1×10⁵ Jurkat-NFAT-luc2 cells/well were added to the 96-well plate and incubated with 50 μL of test antibody for 6 hours. After an incubation of about 6 hours, 80 μL/well ONE-Glo Luciferase Assay buffer was added to the 96-well plate and 100 μL supernatant was collected for measuring luminescence using a plate reader.

As shown in FIGS. 21A-21B, TY25023 demonstrated similar activity to TY24051 in both Jurkat-NFAT reporter assay and cell killing assay. This suggested that the activity of the anti-CD3 arm of the bispecific antibody was not significantly affected by its binding activity.

### D. CD8+ T Cell Killing Assay

The in vitro tumor killing activity of parental, activatable, or cleaved anti-CD3 and anti-HER2 bispecific antibodies against SK-OV-3 (high levels of HER2) or MCF-7 (low levels of HER2) tumor cell lines was measured. Briefly, fresh human PBMCs were isolated from a healthy donor (Donor #116) and CD8+ T cells were prepared from PBMCs using EasySep Human CD8+ T isolation kit. The effector cells (CD8+ T cells) were mixed with target cells (SK-OV-3 cells or MCF-7 cells) in final cell ratio 10:1 CD8+ T cells: Target. Sample dilutions of bispecific antibody were prepared in assay medium consisting of RPM 1640 supplemented with 1% FBS. Plates were placed in a 37° C. incubator supplemented with 5% CO2 for approximately 24 hours. At the end of incubation, 50 μL supernatant was collected for measuring LDH release using a plate reader.

As shown in Table 49, when SKOV-3 was used as target cells, compared with parental TY25023, the tumor killing activity of the activatable antibodies TY25362 and TY25026 was significantly lower (by 200 or 40000-fold reduction). When MCF-7 cells were used as target cells, notably, the two activatable antibodies showed no detectable tumor killing activity. After cleaved by MMP-9, the activities of the activatable antibodies are mostly recovered.

TABLE 49
EC50 and masking efficiency (ME) of different bispecific
antibodies in human CD8+ T cell killing assays

| | SKOV3 | | MCF7 | |
|---|---|---|---|---|
| Antibody | EC50 (nM) | ME | EC50 (nM) | ME |
| TY24051 | 0.00035 | 1 | 0.00271 | 1 |
| TY25023 | 0.00072 | 2.08 | 0.01257 | 4.63 |
| TY25026-Cleaved | 0.00208 | 6 | 0.03989 | 14.7 |
| TY25362-Cleaved | 0.00171 | 4.94 | 0.02827 | 10.42 |
| TY25026 | 13.98 | 40393 | NA | NA |
| TY25362 | 0.07481 | 216.15 | NA | NA |

The level of IFNγ secretion in an activated CD8+ T cell assay in response to treatment with the bispecific antibodies TY24051, TY24052, TY25023, and TY25026 was measured. As shown in FIG. 21C, TY25023 resulted in a significantly increased level of IFNγ secretion by CD8+ T cells compared to TY25026.

Furthermore, as shown in FIG. 21B, both the parental bispecific antibodies TY25023 and TY24051 and their activatable bispecific antibodies TY25026 and TY24052 could induce dose-dependent increases in tumor killing activities. The parental bispecific antibodies TY25023 and TY24051 showed a stronger potency of tumor killing activity than their activatable bispecific antibodies TY25026 and TY24052 did. The activatable antibodies promoted SK-OV3 tumor cell lysis and IFNγ secretion at a level similar to a reference CD3× isotype control antibody.

### E. Cytokine Release Assay

The in vitro cytokine release triggered by parental, activatable, or cleaved anti-CD3 and anti-HER2 bispecific antibodies against MCF-7 (low levels of HER2) tumor cell lines was measured. Briefly, fresh human blood was drawn from healthy donor. RBCs were lysed from heparin-treated healthy donor blood using ammonium chloride potassium (ACK) red cell lysis buffer. Four-hundred thousand whole blood cells were incubated with MCF7 cells and the serially diluted HER2-TDBs for 20 hours in 10:1 E:T ratio. Groups without MCF-7 cells were set as no target cell control. Cytokine release (IL-2 and INF-γ) from supernatants was analyzed by ELISA.

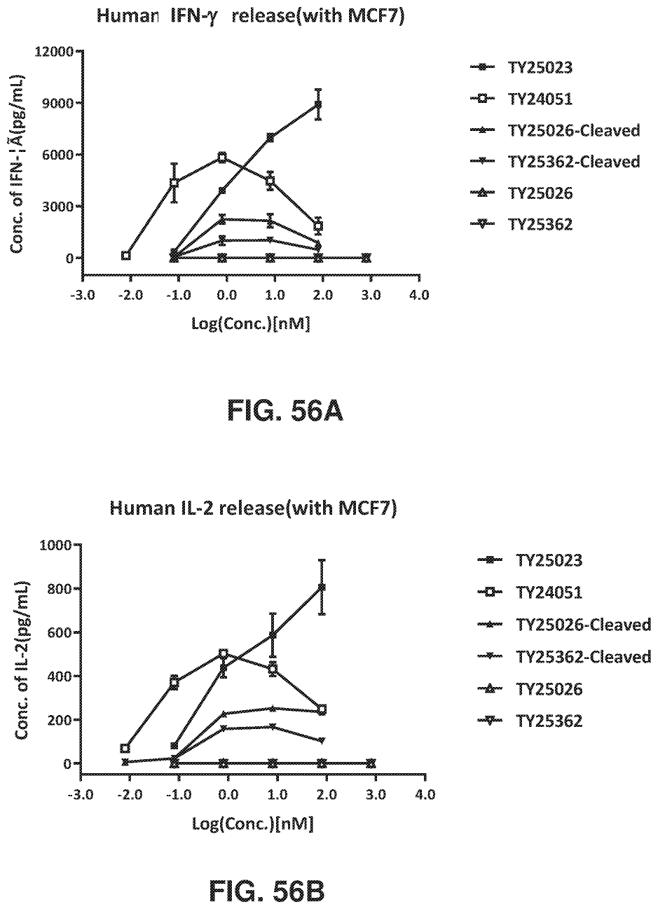

As shown in FIGS. 56A-56B, cytokine release levels (IFN-γ or IL-2) of TY24051 were higher than TY25023 at the concentrations below 0.8 nM. The activatable antibodies (TY25026 and TY25362) showed no detectable cytokine release. In the absence of target MCF-7 cells, all the groups released very low cytokines (data not shown).

F. Efficacy Study of Bispecific Antibodies in EMT6-HER2 Model in M-NSG Mice Transplanted With hPBMC

Human PBMCs (1×10⁷ per mouse) were transplanted into female M-NSG immunodeficient mice by intraperitoneal injection. 9 days later, EMT6 mouse breast cancer cells stably transfected with HER2 were inoculated subcutaneously to these mice. Antibody dosing started when average tumor volume reached about 120 mm³ on day 6 post inoculation. 5 mg/kg of the antibodies TY24051, TY25023, TY25026, TY25362, and an isotype control were administered by intraperitoneal injection, twice a week for 2 weeks, and tumor volume was measured over time.

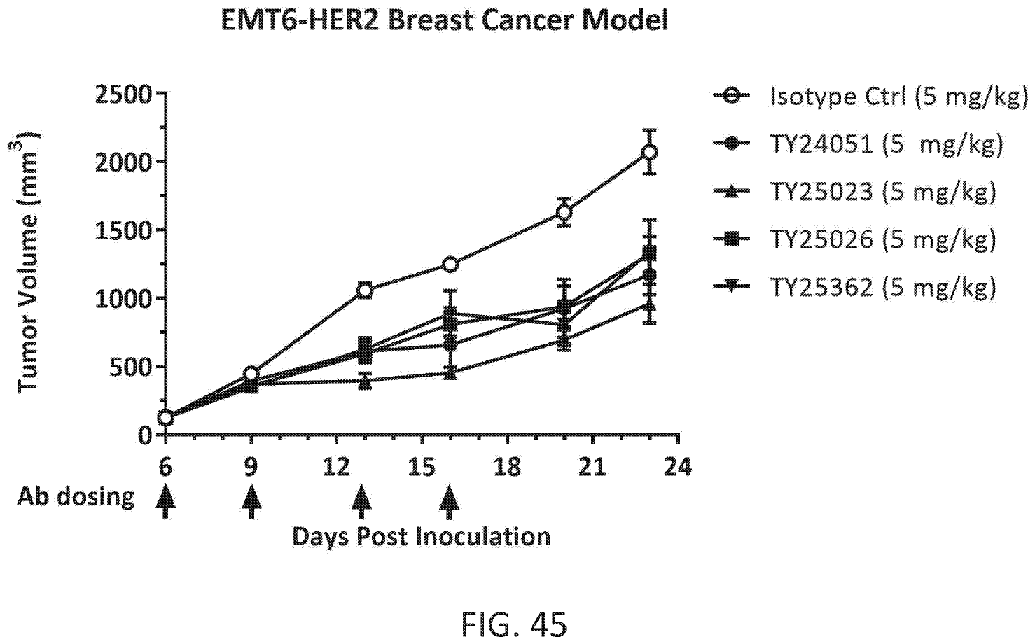

As shown in FIG. 45, parental and SAFE bispecific antibodies were active to inhibit the growth of the HER2 overexpressing EMT6 tumor in human PBMC transplanted M-NSG mice.

### G. HER2 Expression (SK-OV3) Tumor Model

Immunodeficient M-NSG mice (n=6 per group, female, 8-9 weeks old) inoculated subcutaneously with 2×10⁶ SK-OV3 cells. One day later the mice were transplanted with 1×10⁷ PBMC through i.p. injection. Treatment began at Day 15 post tumor inoculation when the average tumor volume reached about 190 mm³. The mice were administered with Vehicle, anti-HER2xanti-CD3 bispecific parental antibody TY25023 or anti-HER2xanti-CD3 bispecific safebodies TY25026 and TY25362 at 5 mg/kg or 1 mg/kg by i.p. injection. The mice were administered these Abs twice per week for a total of four doses. Tumor growth was monitored twice a week and reported as the mean tumor volume±s.e.m. over time. Immunodeficient M-NSG mice (n=6 per group, female, 8-9 weeks old) were transplanted with 5×10⁶ PBMC through i.p. injection. Seven days later, the mice were inoculated subcutaneously with 2×10⁶ SK-OV3 cells. Treatment began at Day 7 post tumor inoculation when the average tumor volume reached about 60 mm³. The mice were administered with Vehicle, anti-HER2xanti-CD3 bispecific parental antibody TY25023 at 1 mg/kg or 0.2 mg/kg, or anti-HER2xanti-CD3 activatable antibodies TY25026 and TY25362 at 5 mg/kg or 1 mg/kg by i.p. injection. The mice were administered these Abs twice per week for a total of three doses. Tumor growth was monitored twice a week and reported as the mean tumor volume±s.e.m. over time.

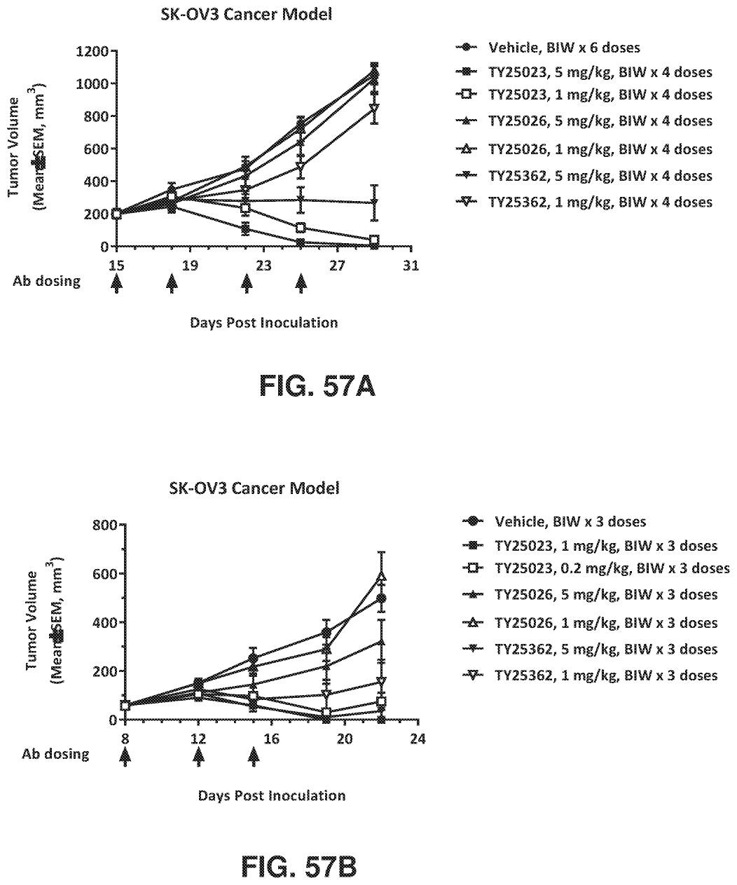

As shown in FIG. 57A, the parental antibody TY25023 showed much stronger anti-tumor efficacy than both TY25026 and TY25362. While TY25362 also showed much stronger anti-tumor efficacy than TY25026. TY25026 showed basically no anti-tumor efficacy in this scenario.

As shown in FIG. 57B, the parental antibody TY25023 showed much stronger anti-tumor efficacy than both TY25026 and TY25362 at 1 mg/kg. TY25362 showed stronger anti-tumor efficacy than TY25026 at both 1 mg/kg and 5 mg/kg. All the three antibodies suppress the tumor growth of SK-OV3 model in a dose-dependent manner.

### Example 7. Toxicity Study of Activatable HER2xCD3 Bispecific Antibody in Cynomolgus Monkeys

The following example describes experiments to evaluate the safety profiles of parental and activatable HER2xCD3 antibodies in cynomolgus monkeys. Specifically, the presence of cytokine and immune-associated events was measured in response to treatment with the low affinity anti-CD3 bispecific antibody TY25023, the corresponding activatable bispecific antibody TY25026, comparison antibody TY24051, and its corresponding activatable bispecific antibody TY24052.

### A. Low Dose Administration

Cynomolgus monkeys were dosed intravenously with TY25023, TY25026, TY24051, or TY24052. Monkeys were dosed with sequential doses of antibody at 0.2 mg/kg on day 1, 0.5 mg/kg on day 8, and 0.9 mg/kg on day 15. A summary of the study design is provided in Table 14 below.

TABLE 14
HER2 × CD3 Parental and SAFE Bispecific
Antibody Study Design in Cyno Monkeys

| Antibody | Cynomolgus | Dosing | Dosing | Dosage |
|---|---|---|---|---|
| treatment | monkey (ID) | schedule | route | (mg/kg) |
| TY24051 | 1 male (1001) | 1 dose | i.v. | 0.2-0.5-0.9 |
| TY24052 | 1 male (1102) | 1 dose | i.v. | 0.2-0.5-0.9 |
| TY25023 | 1 male (1203) | 1 dose | i.v. | 0.2-0.5-0.9 |
| TY25026 | 1 male (1304) | 1 dose | i.v. | 0.2-0.5-0.9 |

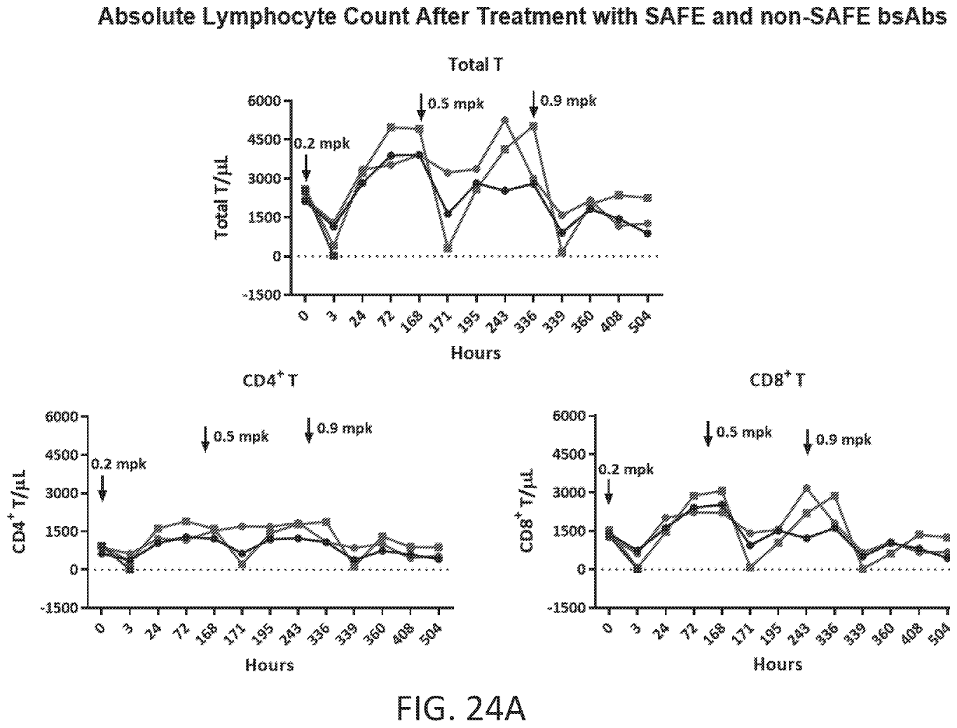

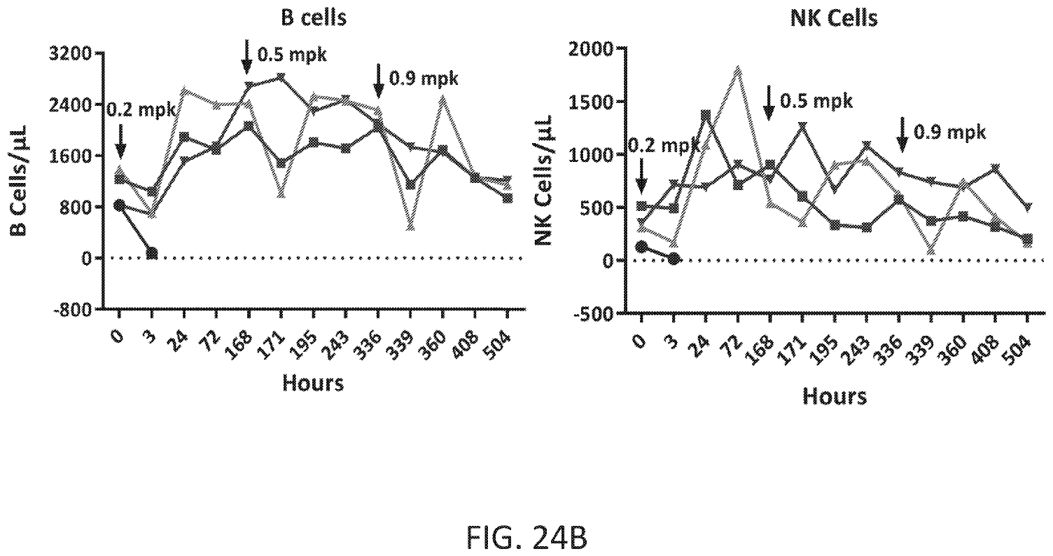

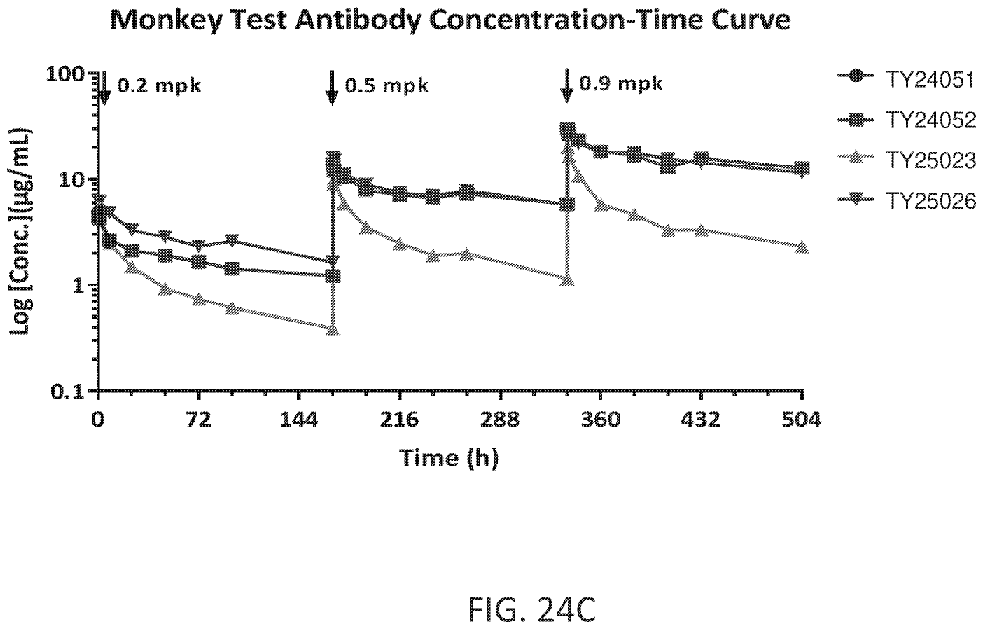

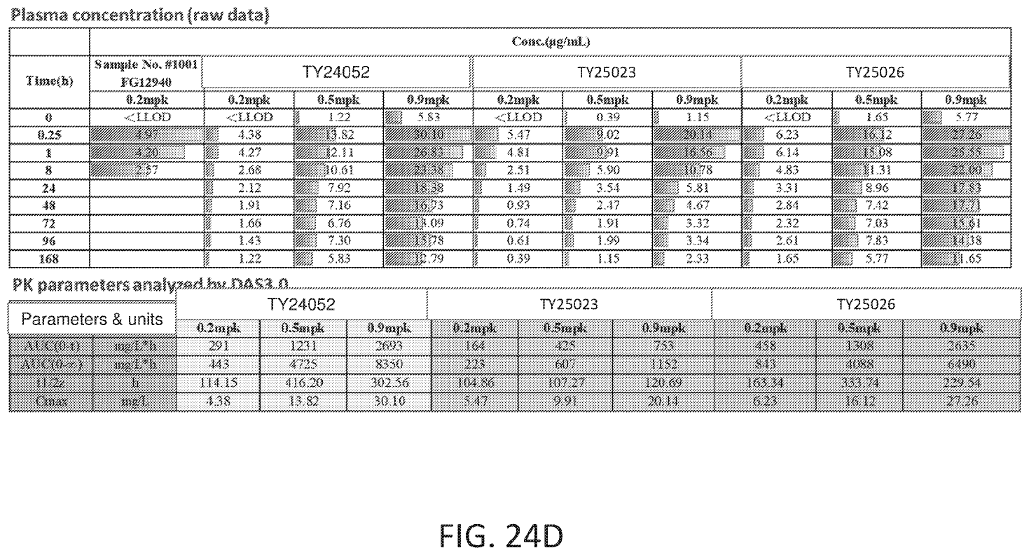

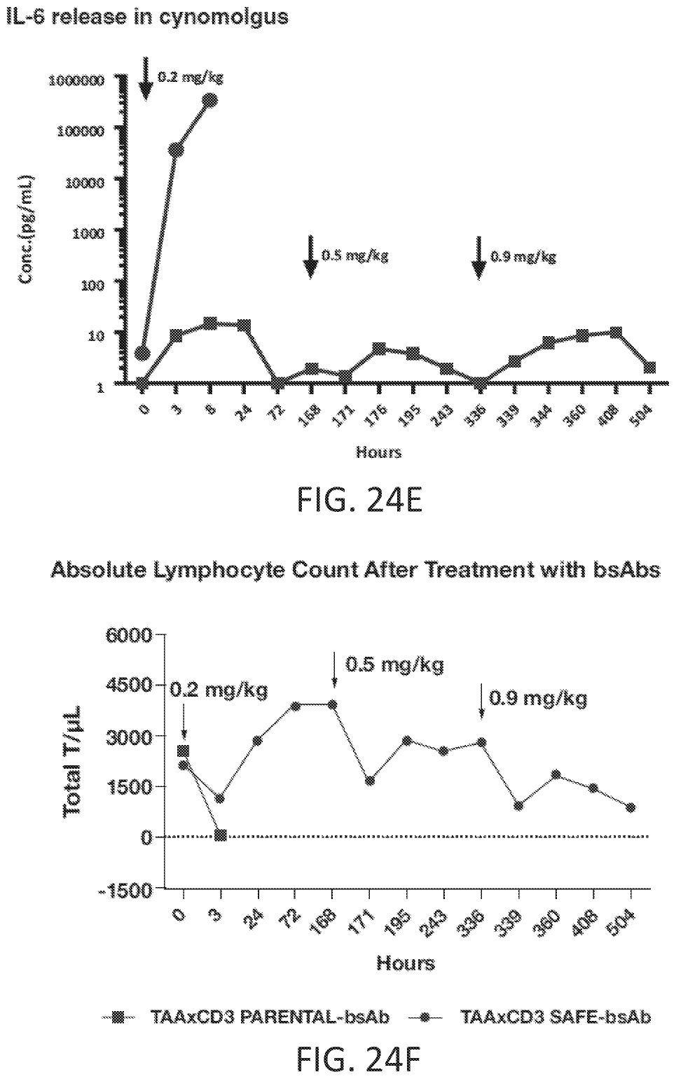

Serum samples were collected to measure cytokine levels, and blood was collected for lymphocyte profiling. Samples were collected at 0, 3, 8, 24, 72, 168, 171, 176, 195, 243, 336, 339, 344, 360, 408 and 504 hours. The level of cytokines IFNγ, IL-2, IL-6, TNFα, IL-5, and IL-4 released was measured over the course of 504 hours following administration (FIGS. 24E-24F). Further, the level of CD4+ and CD8+ T cell activation was measured, as indicated by the percentage of CD69+ T cells (FIG. 23). Finally, the absolute lymphocyte count was measured (FIGS. 24A-24B).

For the CD3 masked only bispecific antibody TY25362, cynomolgus monkeys were intravenously dosed with sequential doses of antibody at 1 mg/kg on day 1, 10 mg/kg on day 8, and 30 mg/kg on day 15. The same toxicity parameters were examined including the presence of cytokine and immune-associated events in response to treatment as described above.

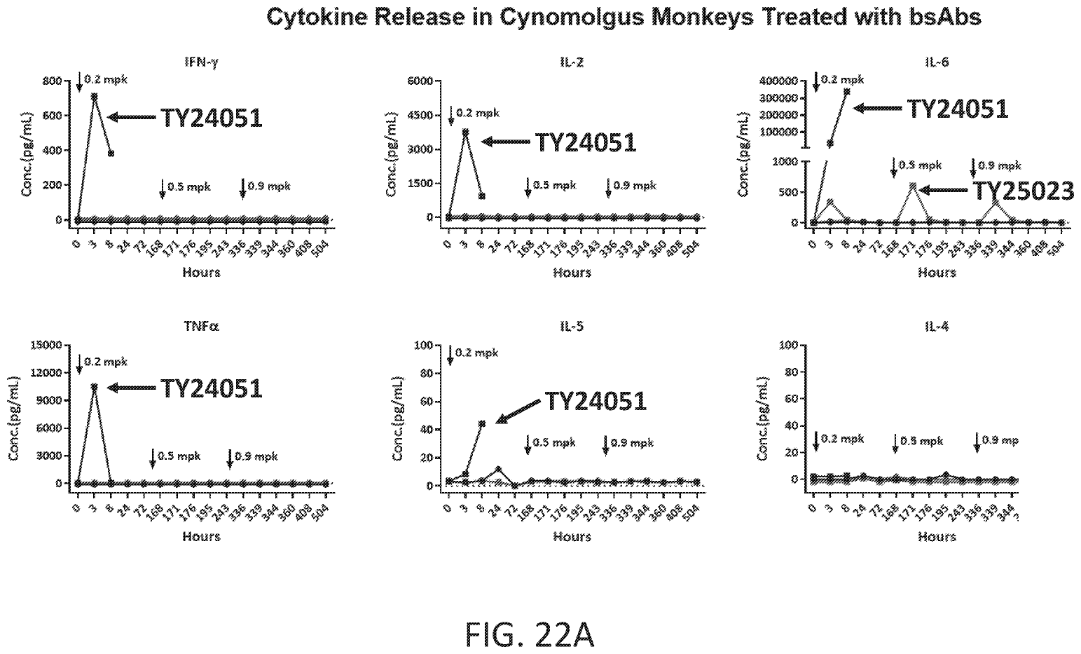

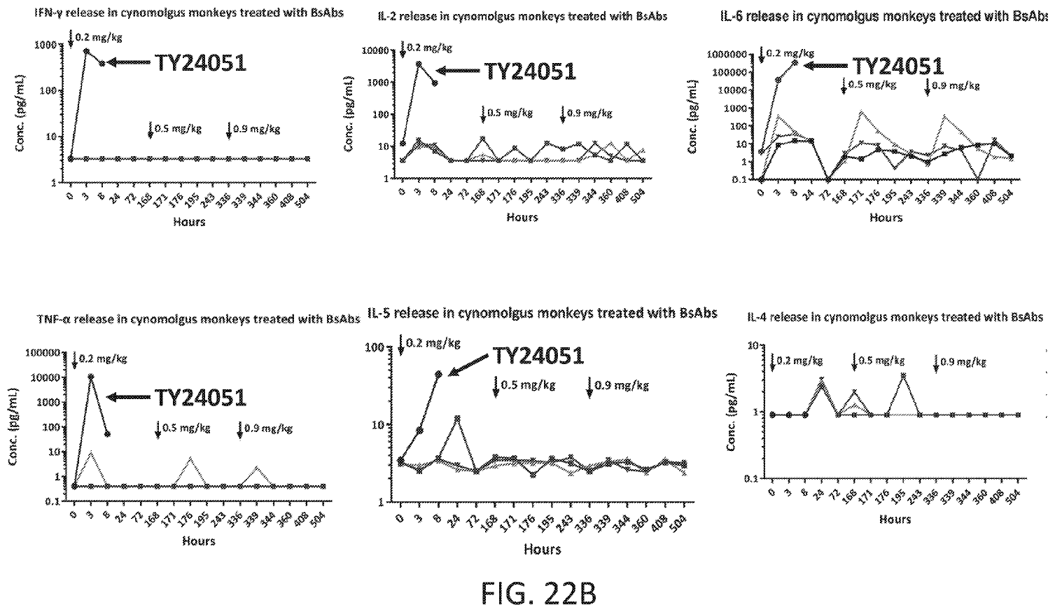

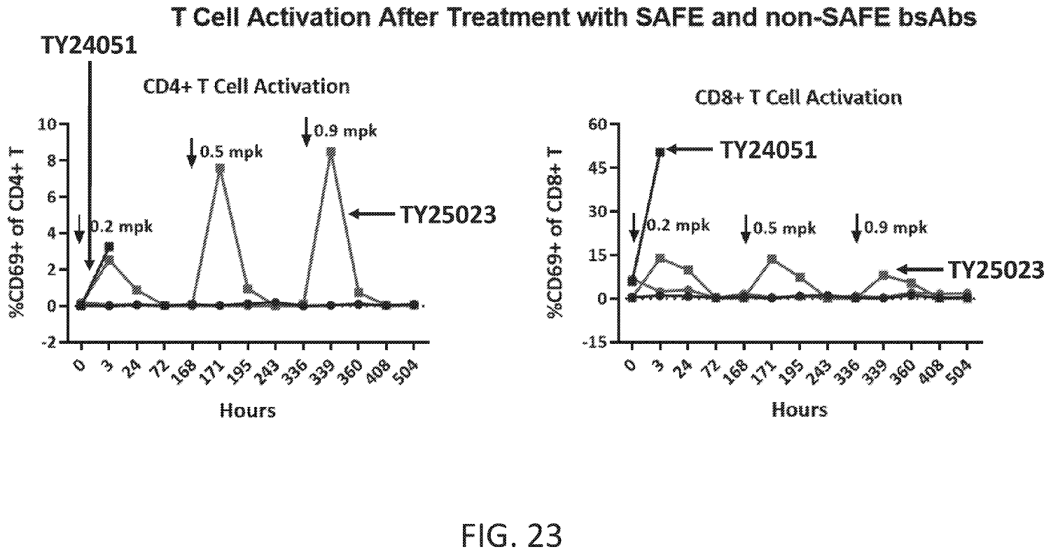

As shown in FIGS. 22A-22B, treatment with TY25026 or TY24052 did not induce significant cytokine release at any of the three dose levels (0.2, 0.5, or 0.9 mg/kg, or “mpk”) during the testing period. These results clearly showed that no induction of cytokine release was observed in cynomolgus monkeys treated with the two SAFEbodies TY25026 and TY24052 at any dose tested, demonstrating a significant improvement in safety profiles by SAFEbody masking. Significant cytokine release was observed with the non-masked bispecific antibody control TY24051. Furthermore, transient release of IL-6 was observed after each dosing of TY25023, indicating weak cytokine induction. Additionally, as shown in FIG. 23, T cell activation was observed after dosing of either TY25023 or TY24051, as manifested by an increase in CD69+ cells in both CD4+ and CD8+ T cell populations at 3 hours after each dosing. No changes in the level of CD69+ cells were observed after dosing with either TY25026 or TY24052.

Consistent with the severe dose limiting toxicities seen with other bispecific T-cell engager antibodies, the cynomolgus monkey treated with the non-masked bispecific antibody reference TY24051, died ˜12 hours after first dosing at 0.2 mg/kg, likely due to acute cytokine release syndrome. Moreover, as shown in FIG. 24A, total T cells, CD4+, and CD8+ T cells were nearly undetectable at 3 hours following the initial TY24051 treatment at 0.2 mg/kg. B cells and NK cells were also depleted (FIG. 24B). A similar phenomenon was observed following TY25023 treatments at all three dose levels, except that the level of lymphocytes rebounded after 24 hours. The two monkeys treated with masked bispecific antibodies exhibited lesser changes in absolute lymphocyte counts.

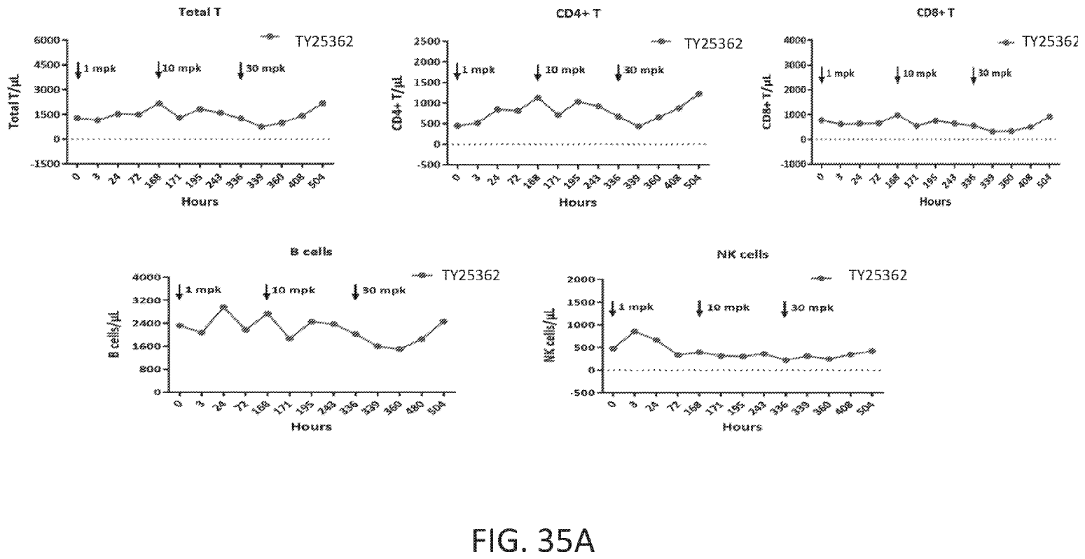

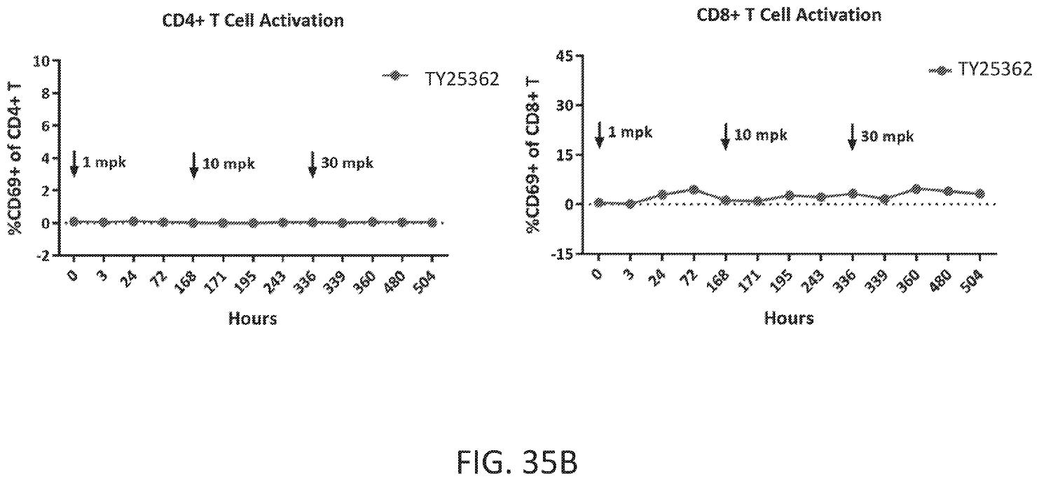

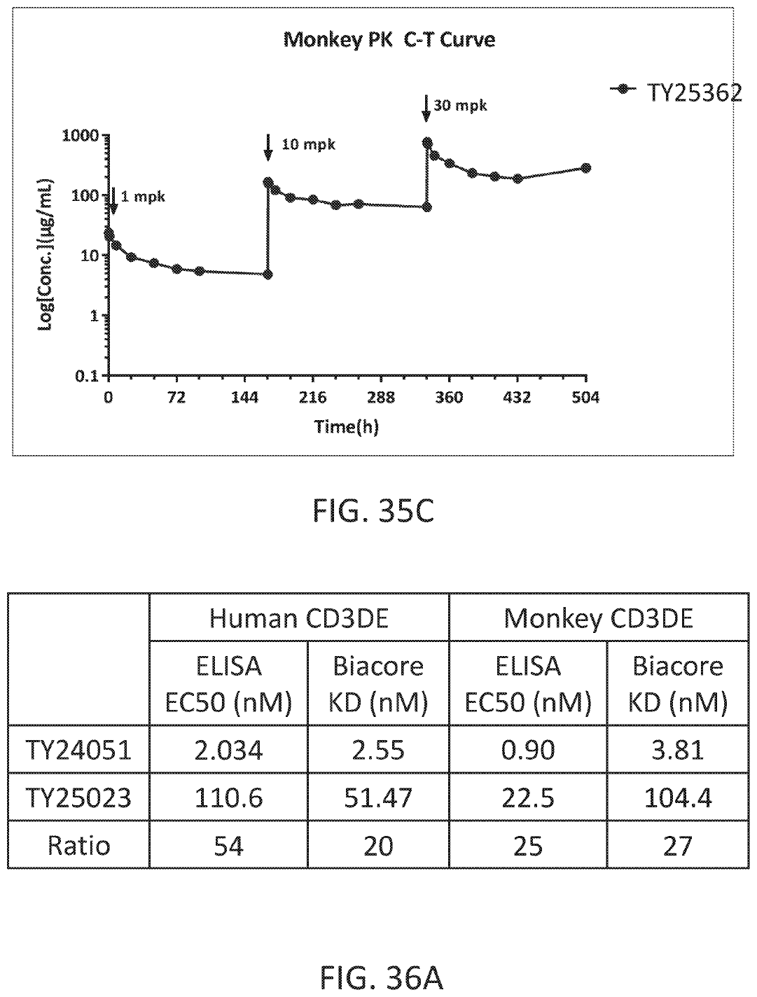

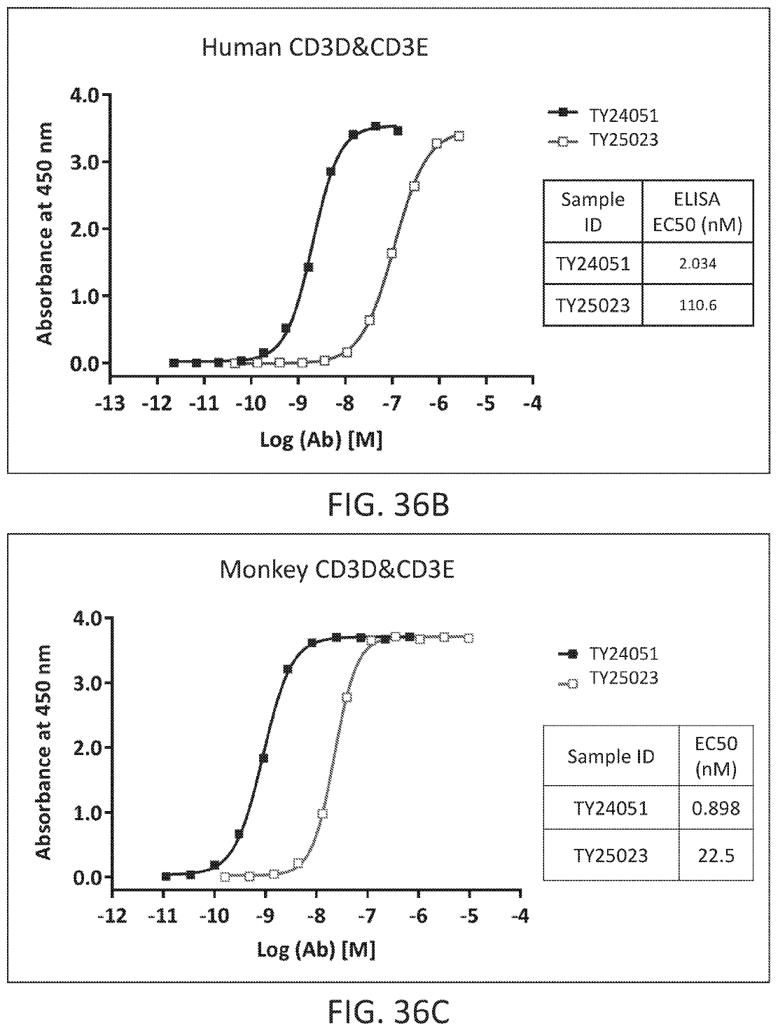

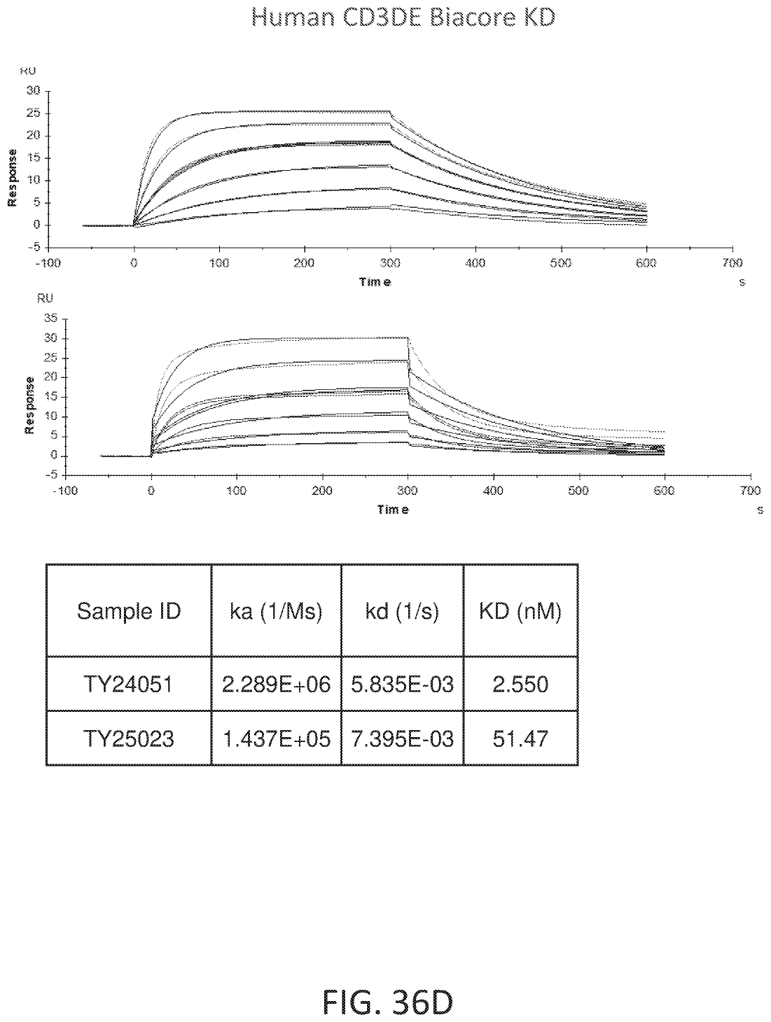

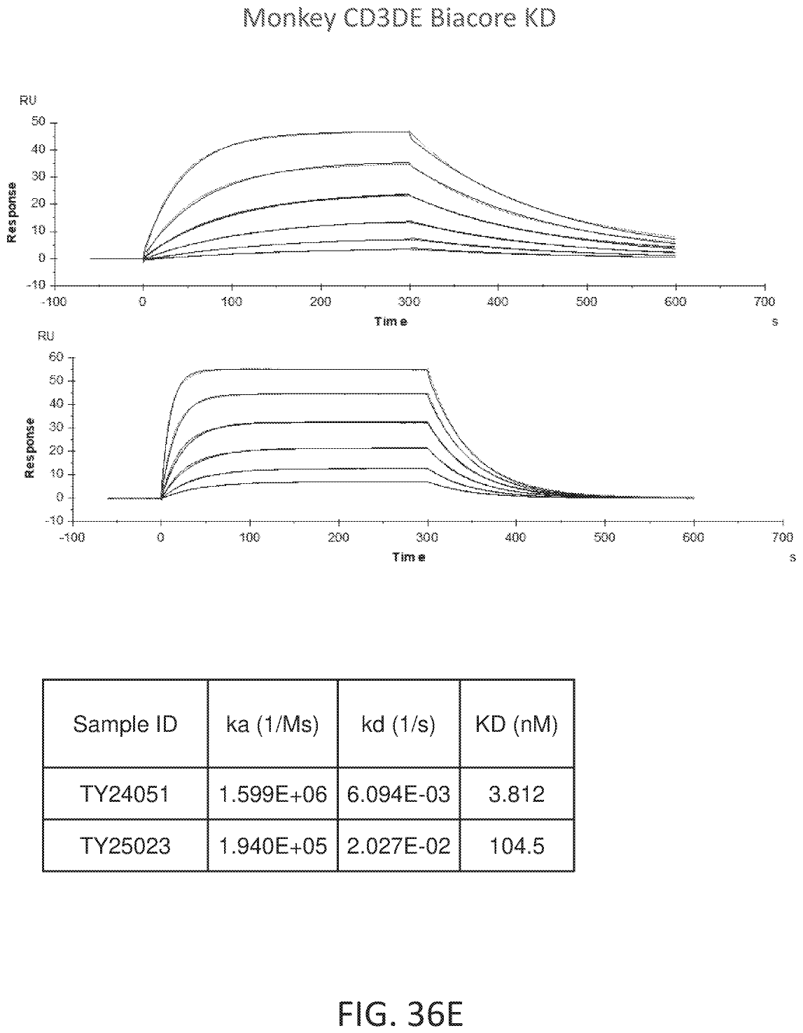

As shown in FIGS. 35A-35B, treatment with TY25362 did not induce significant changes in lymphocyte subpopulations including T, B, and NK cells (FIG. 35A), as well as T cell activation as indicated by CD69+ staining (FIG. 35B). Similar to the other two masked bispecific antibodies TY24052 and TY25062, no cytokine release was detected for TY25362-treated monkeys at any of the three dose levels (1, 10, or 30 mg/kg, or “mpk”) during the testing period (data not shown). These results clearly showed that masking CD3 only can be sufficient for a significant improvement in safety profiles.

### B. High Dose Administration

A pilot non-GLP toxicology and pharmacology study was performed in healthy male cynomolgus monkeys (Macaca fascicularis) to determine the pharmacodynamics and toxicological profile of the CD3xHER2 bispecific antibodies in these animals. Each animal received either a single parental bispecific antibody or activatable antibody, as summarized in Table 51. Animals were observed for clinical signs and clinical pathology parameters prior to and following dosing. Blood was withdrawn immediately prior to dosing in order to establish baseline levels. TY25023, TY25026, and TY25362 were administered by i.v. bolus injection and blood was withdrawn at 0.25 h, 1 h, 8 h, 24 h, 48 h, 72 h, 96 h, 168 h, 240 h, and 336 h post dosing.

Peripheral blood samples were collected up to 168 hours and analyzed by FACS for pharmacodynamics markers (including absolute CD4+ T and CD8+ T cell counts, and measurements of CD69 expression on CD4+ and CD8+ T cells). Serum samples were also collected up to 336 h and analyzed for cytokine levels (IFN-γ, IL-2 and other cytokines) using standard analytical methods.

TABLE 51
HER2 × CD3 Parental and SAFE Bispecific
Antibody Study Design in Cyno Monkeys

| Antibody | Number/Sex | Dosing | Dosing | Dosage |
|---|---|---|---|---|
| treatment | of Animals | schedule | route | (mg/kg) |
| TY25023 | 1 male | Single dose | i.v. | 1 |
| | 1 male | Single dose | i.v. | 3 |
| TY25026 | 1 male | Single dose | i.v. | 30 |
| | 1 male | Single dose | i.v. | 90 |
| TY25362 | 1 male | Single dose | i.v. | 30 |

All treatments were well tolerated in monkeys except TY25023 at 3 mg/kg due to clinical signs observed and remarkable cytokine release.

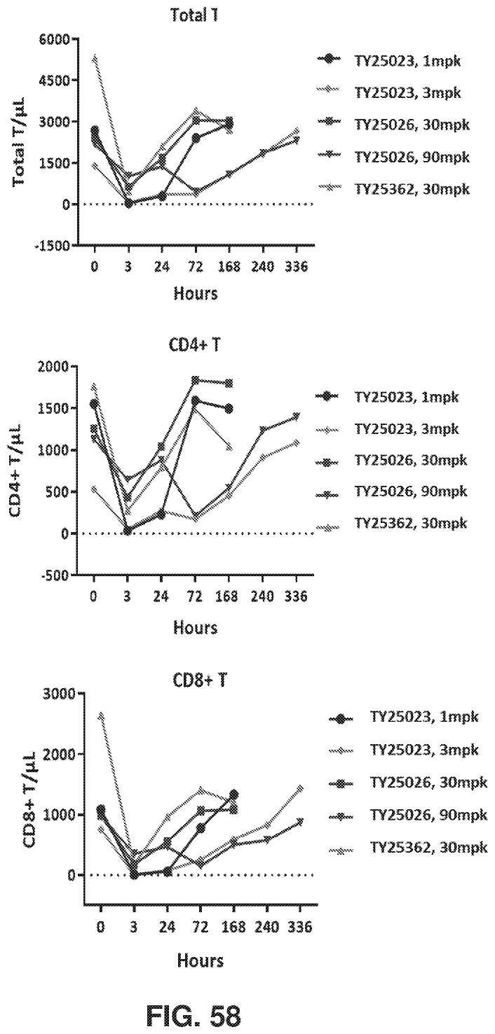

As shown in FIG. 58, CD4+ and CD8+ T cell transmigration was observed 3 hours after antibody injection (both with parental and activatable antibodies). For the parental TY25023, the total T cells including CD4+ and CD8+ T cells declined sharply at 3 hours post dose, consistent with T cell margination. For the activatable antibodies, the T cells also decreased sharply, but the extent was less. The T cell activation profile largely correlated with T cell margination status. At 3 h post dose, the parental antibody can significantly stimulate the T cells to express the CD69 molecules.

As shown in FIG. 59, a significant cytokine storm was observed after TY25023 dosing at 1, and 3 mg/kg. No obvious cytokine release was observed after TY25026 and TY25362 dosing at 30, or 90 mg/kg. Clinical signs were only observed in TY25023-treated animals. Loose feces was noted in the animal receiving 1 mg/kg TY25023 and decreased activity was noted in the animal receiving 3 mg/kg TY25023.

Hematological changes were similar across treatment: depletion of lymphocytes and increase in neutrophils which resolved in approximately 2 days; decreased in red cell mass with compensatory increase in reticulocytes which were still present at the last measured timepoint. Changes in serum chemistry were also similar across treatments: transient increases in ALT, AST, bilirubin, and CK levels.

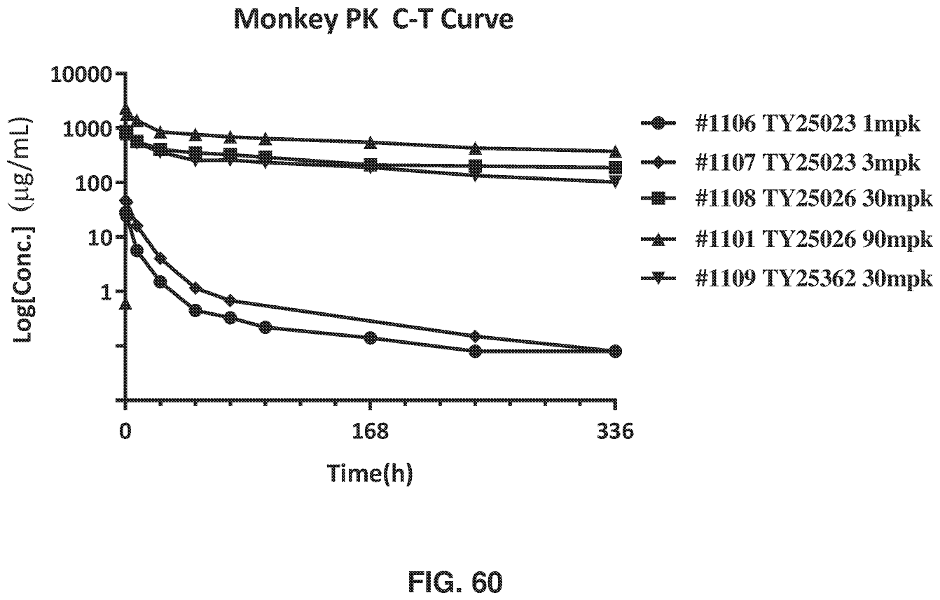

FIG. 60 shows normal PK curves of all the samples. The two activatable antibodies show significantly higher half-life than the parental bispecific antibody

### Example 8. Generation and Biophysical Characterization of Activatable Anti-HER2 Antibodies

The following example describes the generation and characterization of activatable anti-HER2 antibodies derived from the parent antibody trastuzumab.

### A. Display of the Functional Anti-HER2 Antibody on Yeast Surface

A low copy number, CEN/ARS-based vector was used to express a target antibody (antibody trastuzumab, targeting human HER2) under the control of the inducible GAL1-10 promoter in the yeast S. cerevisiae. The surface display of scFvs was achieved through the Aga2 protein fused at its C-terminus under the control of the GAL1 promoter, similar to previously published arrangements (see Boder and Wittrup Nat. Biotechnol. 1997 15(6):553-7). For Fabs, surface display was achieved through the Aga2 protein fused to the N-terminus of the heavy chain (fusion of VH and CH1), under the control of the GAL1 promoter, while the light chain (fusion of VL and CL) was under the control of the GAL10 promoter. The Fabs were displayed on the yeast surface through its association with the membrane-anchored heavy chain.

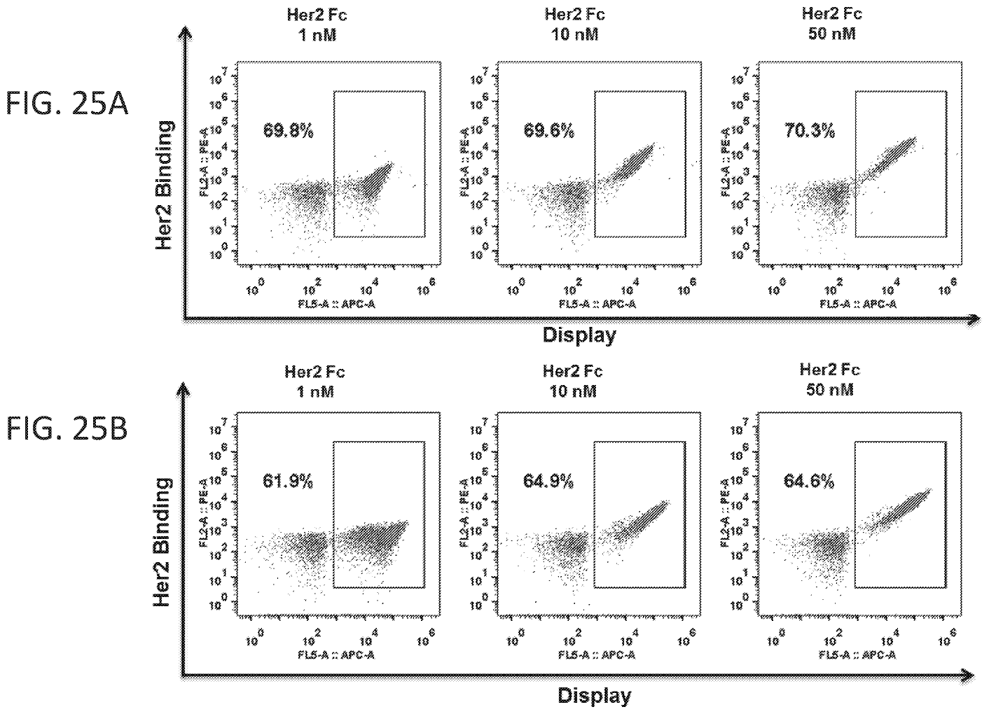

The surface display of the Fab or scFv was verified by staining with antibodies recognizing the fused affinity tag, and the functionality of the Fabs or scFvs displayed on yeast was examined using biotinylated human HER2-Fc. Briefly, 48 hours after induction in galactose medium, yeast cells (1×10⁶) were harvested, washed once with PBSA buffer, and then incubated with 10 nM of biotinylated antigen for 1 hour at room temperature. The yeast cells were then washed twice with PBSA buffer, and incubated with PE conjugated streptavidin (1:500 dilution) (eBioscience #2-4317-87) for 30 minutes at 4° C. The yeast cells were then analyzed by flow cytometry. As shown in FIGS. 25A-25B, both Fabs (FIG. 25A) and scFvs (FIG. 25B) were capable of binding strongly to HER2.

### B. FACS-Based Screening of Masking Peptides Against a Trastuzumab Antibody

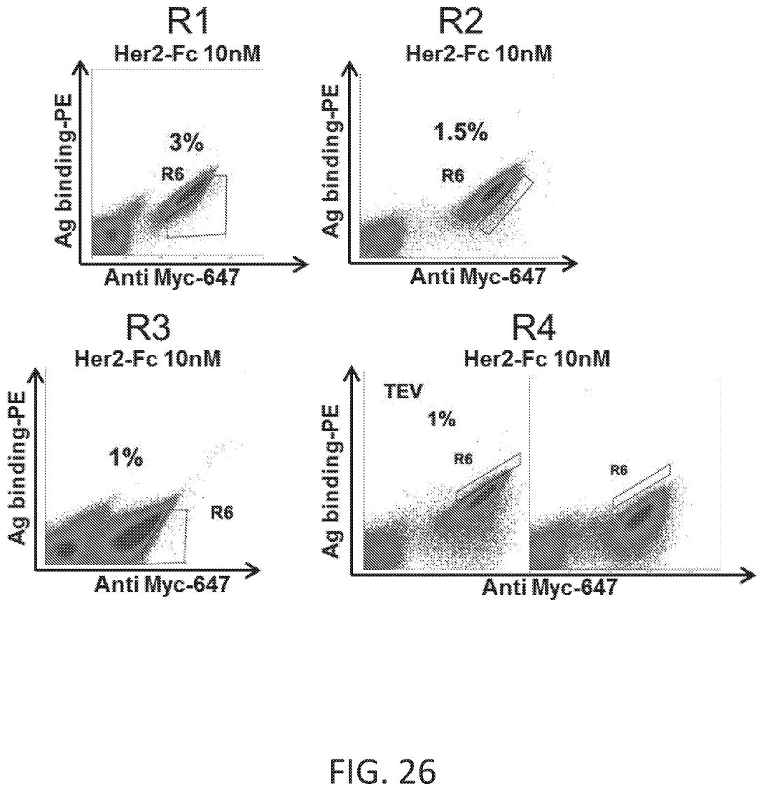

1×10⁸ yeast cells from a CPL yeast library were used to screen for masking peptides against the target antibody. For each round of sorting through MoFlo XDP, yeast cells induced in galactose medium were harvested, washed once with PBSA buffer, and then incubated with 10 nM (decreased to 1 nM in the later rounds) of biotinylated antigen for 1 hour at room temperature. The yeast cells were then washed twice with PBSA buffer, and incubated with PE conjugated streptavidin (1:500 dilution) (eBioscience #2-4317-87) for 30 minutes at 4° C. After two more washes with PBSA buffer, the yeast cells were adjusted to 2-3 OD/mL, and subject to sorting. As shown in FIG. 26, in round 1, 2, and 3, 10 nM of biotinylated HER2-Fc was used, and the weak binders were enriched. The yeast cells from round 3, after growth in glucose medium, were induced in galactose medium and treated with AcTEV protease (6 U/OD cell) (Thermo Fisher Scientific #12575015) for 2 hours at 30° C., and the strong binders were purified, to verify the protease cleavage mediated activation of the target antibody. As shown in FIG. 26, it was apparent that AcTEV cleavage resulted in a dramatic increase of the population of cells that bound strongly to antigen, suggesting that the screening strategy was effective. The single clones from the 4^{th} round of sorting were plated on selective media, and grown individually for further confirmation of cleavage mediated activated antigen binding.

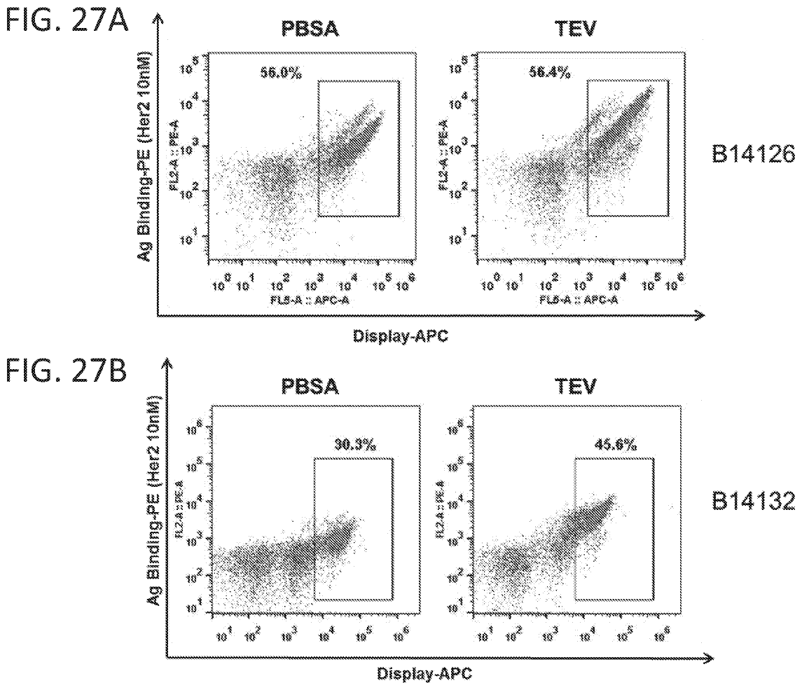

As shown in FIGS. 27A-27B, the selected trastuzumab activatable antibody clones, either in scFv (FIG. 27A) or Fab (FIG. 27B) format, exhibited little binding to antigen in the presence of masking peptide. However, binding to antigen was dramatically increased when the yeast cells were treated with TEV protease to remove the masking peptide. The incorporation of the TEV recognition site in the cleavage peptide, combined with the application of TEV protease to verify the selected clones, significantly increased the success rate of masking peptide selection.

To identify the masking peptide sequences, the shuttle plasmids were extracted from the selected yeast clones (Generay #GK2002-200), and transformed into competent E. coli cells. The plasmids were prepared, and the regions encoding the masking peptides were sequenced and aligned. As anticipated, these sequences could be separated into several groups based on the number of residues between the two cysteine residues in the masking sequence, indicating clear enrichment through rounds of sorting. Four groups of masking peptide sequences (CXₙC, where n=5, 6, 7, or 8), together with the invariant cleavage peptide sequences, are listed in Table 15. In Table 15, the cleavage peptide sequences (SGRSAGGGGTPLGLAGSGGS, SEQ ID NO: 431) are underlined.

TABLE 15
Masking peptide sequences

| | | | SEQ ID |
|---|---|---|---|
| | | | NO of |
| | Peptide | | masking |
| IgG ID | ID | Masking + cleavage peptide sequences: | peptide |
| TY22837 | B13567 | EVGSYHYASDACDADPFDCNASGRSAGGGGTPLGLAGSGGS | 432 |
| TY22838 | B13569 | EVGSYAYIPPDCHADPYDCSVSGRSAGGGGTPLGLAGSGGS | 433 |
| TY22839 | B13572 | EVGSYANTNDPCTLDPYDCSHSGRSAGGGGTPLGLAGSGGS | 434 |
| TY22840 | B13573 | EVGSYTASNADCPYDPYTCYASGRSAGGGGTPLGLAGSGGS | 435 |
| TY22841 | B13580 | EVGSYADALYDCYDADPDCYYSGRSAGGGGTPLGLAGSGGS | 436 |
| TY22842 | B13582 | EVGSYSFSDFACDTTPFDCFASGRSAGGGGTPLGLAGSGGS | 437 |
| TY23446 | B14104 | EVGSYITPDCLFFDPFDCVDNSGRSAGGGGTPLGLAGSGGS | 438 |
| TY23447 | B14109 | EVGSYAADYCDPFDFSFCLSTSGRSAGGGGTPLGLAGSGGS | 439 |
| TY23448 | B14110 | EVGSYPIAICAPHSSDDCAFTSGRSAGGGGTPLGLAGSGGS | 440 |
| TY23449 | B14122 | EVGSYHLALCLDPDDSSCNFASGRSAGGGGTPLGLAGSGGS | 441 |
| TY23450 | B14124 | EVGSYSSTACIFIDPFDCSVASGRSAGGGGTPLGLAGSGGS | 442 |
| TY23451 | B14126 | EVGSYDHDNYDCYDYYDNCYYSGRSAGGGGTPLGLAGSGGS | 443 |
| TY23452 | B14128 | EVGSYPFLVCDDASPFDCTLVSGRSAGGGGTPLGLAGSGGS | 444 |
| TY23453 | B14131 | EVGSYIAFYCPDAHPYDCTSLSGRSAGGGGTPLGLAGSGGS | 445 |
| TY23454 | B14132 | EVGSYPALDCATFPSAVCTADSGRSAGGGGTPLGLAGSGGS | 446 |
| TY23523 | B14165 | EVGSYAPADFDCSVFADPFDCSSSGRSAGGGGTPLGLAGSGGS | 447 |
| TY23524 | B14249 | EVGSYNDYDHYCSAFPSDLACANSGRSAGGGGTPLGLAGSGGS | 448 |
| TY23525 | B14253 | EVGSYDPTTDVCSYYSDPFDCYYSGRSAGGGGTPLGLAGSGGS | 449 |
| TY23526 | B14245 | EVGSYDSNDDYCAHDSDPYDCYYSGRSAGGGGTPLGLAGSGGS | 450 |
| TY23527 | B14246 | EVGSYPAPAYNCHATSDPYDCANSGRSAGGGGTPLGLAGSGGS | 451 |
| TY23528 | B14247 | EVGSYDNDNYDCYDHHNDHDCYYSGRSAGGGGTPLGLAGSGGS | 452 |
| TY23529 | B14248 | EVGSYYDDDDDCYDNYYDHDCYYSGRSAGGGGTPLGLAGSGGS | 453 |
| TY23530 | B14254 | EVGSYFDLHDYCNTYHDFPDCAPSGRSAGGGGTPLGLAGSGGS | 454 |
| TY23531 | B14256 | EVGSYYADYTACAFANSPYDCDYSGRSAGGGGTPLGLAGSGGS | 455 |
| TY23532 | B14257 | EVGSYHADHANCVSSFDPYDCDNSGRSAGGGGTPLGLAGSGGS | 456 |
| TY23533 | B14261 | EVGSYNHPIDDCYNINDPYDCDHSGRSAGGGGTPLGLAGSGGS | 457 |
| TY23534 | B14262 | EVGSYPHDYHNCYDYYHYDHCHHSGRSAGGGGTPLGLAGSGGS | 458 |
| TY23535 | B14263 | EVGSYAHDYADCLHNSDPYDCPDSGRSAGGGGTPLGLAGSGGS | 459 |
| TY23536 | B14264 | EVGSYYNSDDDCASHYDPYTCYYSGRSAGGGGTPLGLAGSGGS | 460 |
| TY23537 | B14265 | EVGSYYDYSHDCHDLDHNHACDPSGRSAGGGGTPLGLAGSGGS | 461 |
| TY23538 | B14266 | EVGSYTHASDFCHTSIDPYDCAYSGRSAGGGGTPLGLAGSGGS | 462 |
| TY23539 | B14360 | EVGSYAALPLCFPLSFCFVPTSGRSAGGGGTPLGLAGSGGS | 463 |
| TY23540 | B14366 | EVGSYAPPNLCAVVLSCVVAPSGRSAGGGGTPLGLAGSGGS | 464 |
| TY23541 | B14369 | EVGSYTAIPFCPPAIFCDPFSSGRSAGGGGTPLGLAGSGGS | 465 |
| TY23542 | B14220 | EVGSYYDDYHDCDYYDDYDNCYDSGRSAGGGGTPLGLAGSGGS | 466 |
| TY23543 | B14224 | EVGSYSAPHSCSPFSACTPADSGRSAGGGGTPLGLAGSGGS | 467 |
| TY23544 | B14233 | EVGSYYPDHDDCDDYHDHCDYSGRSAGGGGTPLGLAGSGGS | 468 |
| TY22837 | B13567 | EVGSYHYASDACDADPFDCQASGRSAGGGGTPLGLAGSGGS | 469 |
| TY22843 | B13583 | EVGSYQTTSDTCSDADDTCSVSGRSAGGGGTPLGLAGSGGS | 470 |
| TY23521 | B14155 | EVGSYHYDDYDCDDDHDDYYCQYSGRSAGGGGTPLGLAGSGGS | 471 |
| TY23522 | B14158 | EVGSYHYDNYQCTNYDYDYDCADSGRSAGGGGTPLGLAGSGGS | 472 |

### C. IgG Conversion and Expression

The heavy and light chains were cloned into the mammalian expression vector pCDNA3.3 (Thermo Fisher Scientific, cat #K830001) separately, and the masking peptides and the invariant cleavage peptide were fused to the N-terminus of the light chain in the same manner as displayed on yeast surface. All masking peptides listed in Table 15 were converted into IgG1s. The parental anti-HER2 antibody trastuzumab was used; the CDR, VH, VL, and heavy and light chain sequences of trastuzumab are described in Tables 10-12, above.

Pairs of plasmids were transiently transfected into HEK293F cells. After six days, the supernatants were harvested, cleared by centrifugation and filtration, and IgGs were purified with standard protein A affinity chromatography (MabSelect SuRe, GE Healthcare). The IgGs were eluted and neutralized, and buffer exchanged into storage buffer (20 mM histidine, pH 5.5). Protein concentrations were determined by UV-spectrophotometry, and IgG purity was analyzed under denaturing, reducing and non-reducing conditions by SDS-PAGE or SEC-HPLC. Importantly, the expression levels of most of the activatable antibodies in HEK293 cells were similar to their parental antibody, as were their purification yields after protein A resin purification, suggesting that the presence of the masking and cleavage peptides do not have a negative impact on antibody expression in mammalian cells.

### D. Measurement of Masking Efficiency

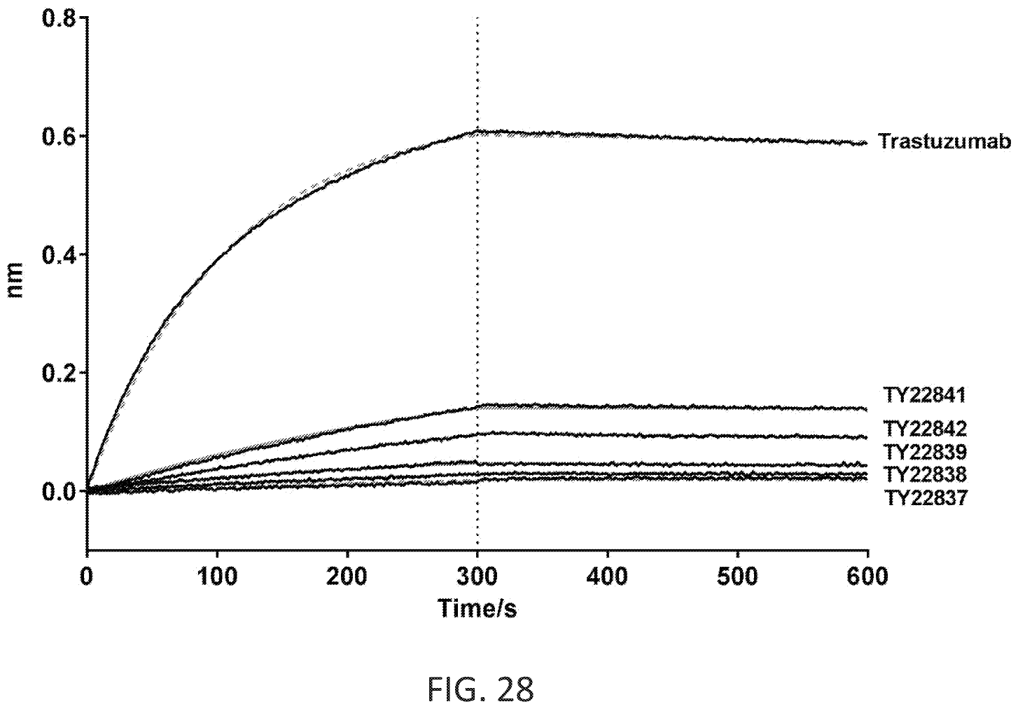

The ForteBio Octet RED96 system (Pall, USA) was used to assess the efficiency of the masking peptides by Biolayer Inferometry. Briefly, activatable antibodies (and their parent antibody, trastuzumab) were diluted to 30 μg/mL in KB buffer (PBS buffer supplemented with 0.02% Tween 20 and 0.1% BSA), and captured by anti-Human IgG Capture (AHC) Biosensors (Pall, USA) in parallel. The sensors were then allowed to associate with His-tagged HER2 protein (25 nM) for 300 seconds, and then dissociate in KB buffer for another 300 seconds. The association and dissociation curves were fitted to a 1:1 Langmuir binding model using ForteBio Data Analysis 7.1 (Pall, USA) according to the manufacturer's guidelines. As shown in FIG. 28, the responses achieved with the activatable antibodies were significantly lower than that for the parent antibody, suggesting that masking peptides effectively blocked the binding of the antibody to its antigen. Among the five activatable antibodies, TY22837 and TY22838 were more effective, consistent with the results from the ELISA assay discussed below.

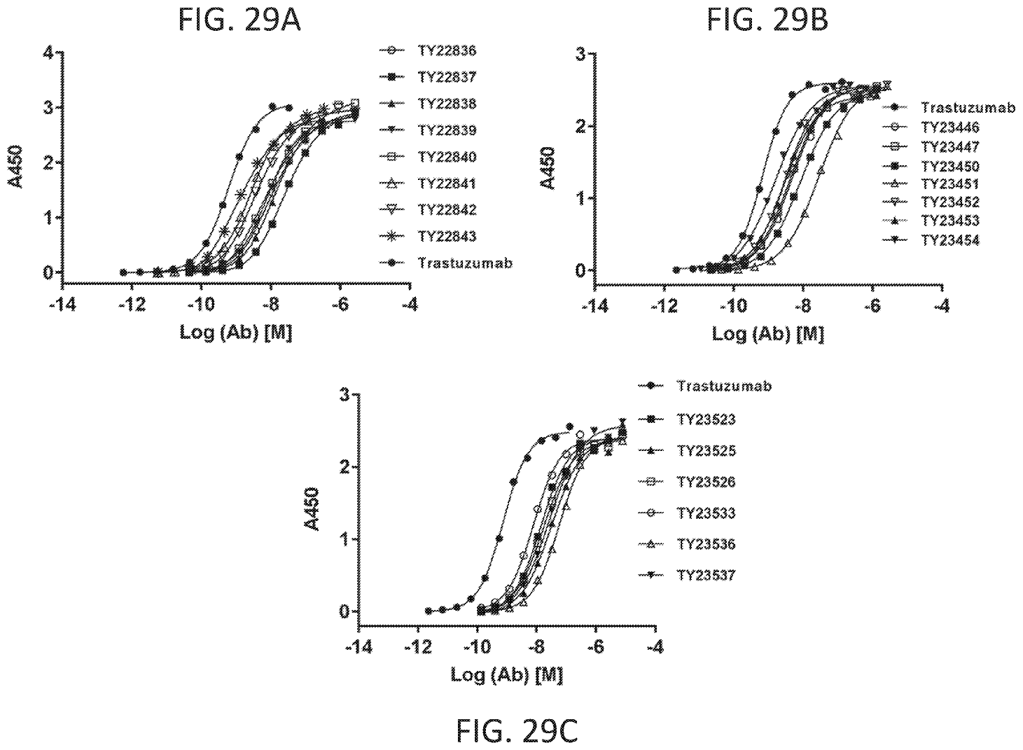

Recombinant human HER2-Fc was diluted to 1 μg/mL in PBS and coated on a Maxisorp plate at 4° C. overnight. Plates were blocked with PBS supplemented with 3% non-fat milk at 37° C. for 1 hour. After washing, 100 μL of 3-fold serial dilutions of antibodies were added to each well. After incubation at 37° C. for 1 hour, plates were washed four times, and 100 μL HRP conjugated anti-human IgG (Fab specific) (1:6000 dilution) was added to each well. Plates were incubated at 37° C. for 1 hour, washed four times, and then 50 μL TMB substrate solution was added to each well, and the plate was incubated at room temperature. Absorbance at 450 nm was measured after the reactions were stopped with 50 μL H₂SO₄ per well. The EC₅₀ was evaluated by fitting the ELISA data using the asymmetrical sigmoidal (four-parameter logistic equation) model of GraphPad Prism 6 software. Masking efficiencies for each activatable antibody were calculated by dividing the EC₅₀ for binding of the activatable antibody by the EC₅₀ of the parental antibody (trastuzumab). As shown in FIGS. 29A-29C and Table 16, compared with the parental antibody, all of the activatable antibodies showed dramatically reduced binding to its antigen, and the calculated masking efficiency ranged from 2 to 79. These results indicated that multiple masking peptides identified from the CPLs maintained their masking efficiency when expressed in mammalian cells, and as part of a full IgG molecule. In Table 16, the masking efficiency was calculated relative to trastuzumab.

TABLE 16
Activatable antibody ELISAs prior to protease cleavage

| | | EC₅₀ | | | Masking |
|---|---|---|---|---|---|
| IgG ID | LogEC₅₀: | M: | nM: | R²: | efficiency: |

Data Batch 1

| | | | | | |
|---|---|---|---|---|---|
| trastuzumab | −9.215 | 6.10E−10 | 0.61 | 0.9992 | 1 |
| TY22836 | −7.913 | 1.22E−08 | 12.2 | 0.9989 | 20 |
| TY22837 | −7.557 | 2.77E−08 | 27.7 | 0.9993 | 45 |
| TY22838 | −7.876 | 1.33E−08 | 13.3 | 0.9987 | 22 |
| TY22839 | −8.097 | 8.00E−09 | 8.00 | 0.9993 | 13 |
| TY22840 | −7.934 | 1.17E−08 | 11.6 | 0.9985 | 19 |
| TY22841 | −8.614 | 2.43E−09 | 2.43 | 0.9995 | 4 |
| TY22842 | −8.373 | 4.24E−09 | 4.24 | 0.9989 | 7 |
| TY22843 | −8.715 | 1.93E−09 | 1.93 | 0.9992 | 3 |

Data Batch 2

| | | | | | |
|---|---|---|---|---|---|
| trastuzumab | −9.167 | 6.80E−10 | 0.68 | 0.9992 | 1 |
| TY23446 | −8.311 | 4.89E−09 | 4.89 | 0.9990 | 7 |
| TY23447 | −8.365 | 4.31E−09 | 4.31 | 0.9996 | 6 |
| TY23450 | −8.068 | 8.56E−09 | 8.56 | 0.9994 | 13 |
| TY23451 | −7.546 | 2.84E−08 | 28.4 | 0.9986 | 42 |
| TY23452 | −8.522 | 3.00E−09 | 3.00 | 0.9967 | 4 |
| TY23453 | −8.535 | 2.92E−09 | 2.92 | 0.9975 | 4 |
| TY23454 | −8.786 | 1.64E−09 | 1.64 | 0.9976 | 2 |

Data Batch 3

| | | | | | |
|---|---|---|---|---|---|
| trastuzumab | −9.123 | 7.53E−10 | 0.75 | 0.9984 | 1 |
| TY23523 | −7.848 | 1.42E−08 | 14.20 | 0.9961 | 19 |
| TY23525 | −7.487 | 3.26E−08 | 32.6 | 0.9935 | 43 |
| TY23526 | −7.752 | 1.77E−08 | 17.7 | 0.9977 | 24 |
| TY23533 | −8.106 | 7.84E−09 | 7.84 | 0.9975 | 10 |
| TY23536 | −7.225 | 5.95E−08 | 59.5 | 0.9994 | 79 |
| TY23537 | −7.556 | 2.78E−08 | 27.8 | 0.9978 | 37 |

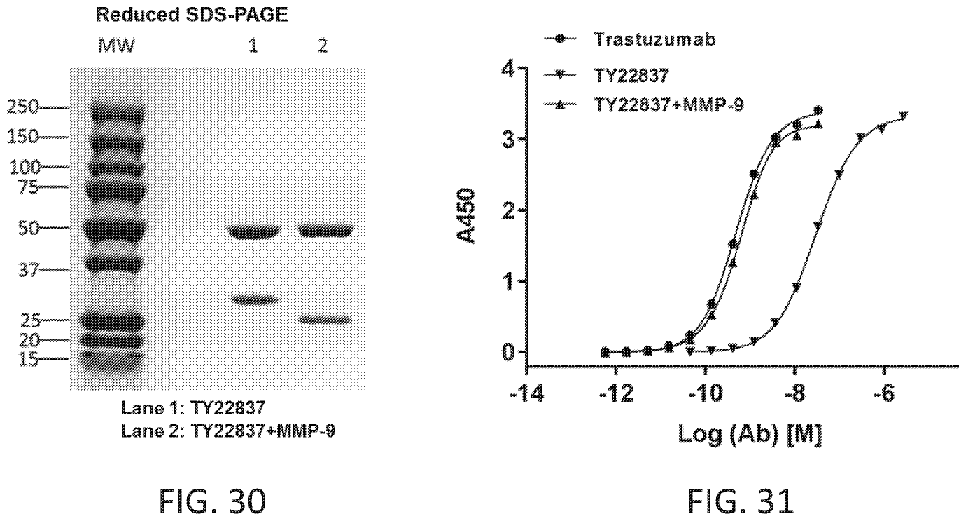

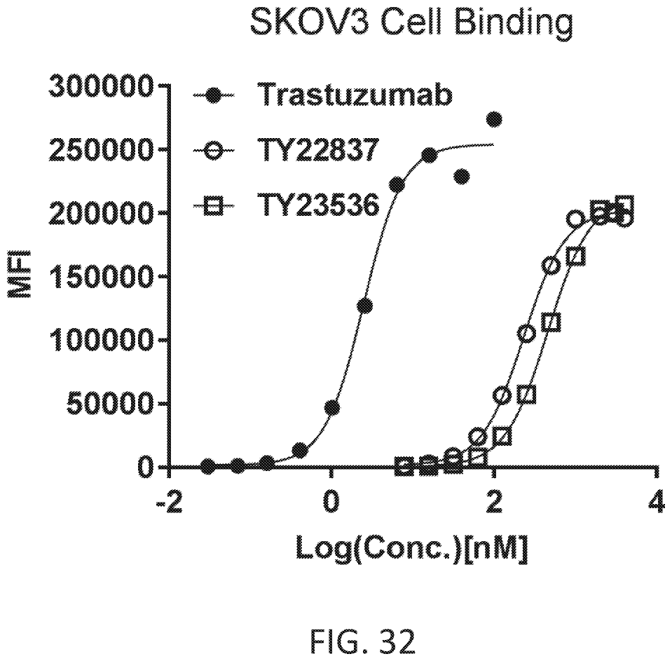

The masking efficiency was also measured through FACS-based assay using SK-OV-3 cell lines. SK-OV-3 cells were detached with Trypsin-EDTA, and resuspended in growth medium. The cells were counted and appropriate amount of the cell suspension according to the counting results was taken out for binding assay. The cells were washed with 2 mL 2% FBS DPBS, centrifuged and resuspended with 2% FBS DPBS to adjust the cell density to 2×10⁶ cells/mL. Antibody serial titrations were prepared by diluting the test antibodies in 96 well plate with 2% FBS DPBS to make 12-points serial dilutions (2× work concentration). 50 μL/well of the cell suspension was aliquoted, so the final cell amount is 1.0×10⁵ cells/well. Then 50 μL/well of antibody serial dilutions was suspended to the corresponding wells containing 50 μL of the cell suspension and incubated at 4° C. for 1 h, protected from light. Afterwards, the plate was centrifuged for 5 minutes at room temperature, washed with 2% FBS DPBS once. The cells were resuspended with 100 μL PE-mouse Anti Human IgG Fc secondary antibody which is diluted to 1 μg/mL with 2% FBS DPBS, and incubated at 4° C. for 30 min, protected from light. Cells were washed, resuspended and transferred to a 96-well flat bottom plate for flow cytometry analysis. FIG. 30 shows an example of FACS curves for the measurement of masking efficiency.

### E. Removal of the Masking Peptide Restores Antibody Activity

The purified activatable antibodies were treated with the proteases that recognize the cleavage sequences, and were then tested to determine whether removal of the masking peptide restored their activity. As an example, 20 μg of TY22837 (0.5 mg/mL) was treated with 10 units of recombinant human MMP-9 (BioVision, #7867-500) in reaction buffer (50 mM Tris, 150 mM NaCl, 5 mM CaCl₂), 20 μM ZnCl₂, pH 7.5). The reactions were carried out at 37° C. for 21 hours. The masking peptides were confirmed to be removed from the light chain by SDS-PAGE analysis FIG. 30. The masking efficiency was then measured by ELISA as described above. As shown in FIG. 31 and Table 17, after removal of masking peptide, the activatable antibody became indistinguishable from the parent antibody in its binding to the antigen. In Table 17, the masking efficiency was calculated relative to trastuzumab.

TABLE 17
Activatable antibody ELISAs after protease cleavage

| | | EC₅₀ | |
|---|---|---|---|
| Sample ID | LogEC₅₀: | nM: | Masking efficiency: |
| trastuzumab | −9.310 | 0.4894 | 1.0 |
| TY22837 | −7.524 | 29.91 | 61 |
| TY22837 + MMP-9 | −9.222 | 0.5992 | 1.2 |

The activatable antibody was also activated by isolated, activated neutrophils. Neutrophils were isolated from heparinized human peripheral blood by gradient centrifugation with Histopaque 1077 to get a mixture of red cells and neutrophils at the bottom of centrifuge tubes, followed by red cell lysis with human red cell lysis reagent (BD, Cat #55899). After washing, neutrophils were induced to release their granule contents by incubating for 2 hours at 37° C. with 160 nM PMA in serum-free RPMI1640, with a cell density of 1×10⁷ cells/mL. After incubation, the cell-free supernatant (which mainly contains the protease MMP-9) was collected by centrifuging at 1200 g for 10 minutes at 4° C. and stored at −80° C. for future use. Then activatable antibodies were incubated with this neutrophil supernatant for certain periods of time, and the cleavage was confirmed by SDS-PAGE. More activatable light chain was cleaved in lane 1 than in lane 2. Without wishing to be bound by theory, this likely indicates that neutrophil secretion of MMP-9 shows more activity at 37° C. than room temperature.

### F. Activatable Antibody Developability Profiles

For manufacturing purpose, it is critical that the discovered activatable antibodies have a good developability profile. Several different tests were performed with purified activatable antibodies that were expressed in mammalian cells. The activatable antibodies were adjusted to 1 mg/mL in 20 mM Histidine, pH 5.5, and antibody quality analysis was performed using analytical size-exclusion chromatography using a Waters 2695 with a Waters 2996 UV detector and aTSKgel g3000 SWXL column (300 mm×7.8 mm) (Tosoh Bioscience). For each assay, 10 g of antibody was injected, and fractionation was performed at a flow rate of 0.5 mL/min in buffer (200 mM sodium phosphate at pH 7.0).

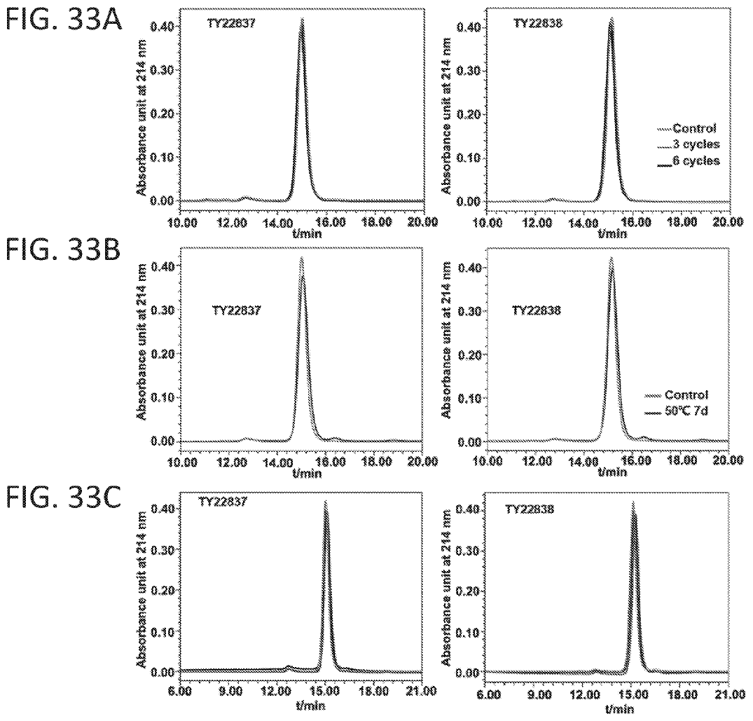

Three accelerated stress tests of TY22837 and TY22838 were conducted: incubation of the activatable antibodies at 50° C. for 7 days, incubation of the activatable antibodies at 40° C. for 28 days, and six cycles of freeze-thaw. The freeze-thaw tests were conducted by freezing 100 μL sample (1 mg/mL in 20 mM histidine, pH 5.5) at −80° C. for 30 minutes, followed by thawing at room temperature for 60 min. As shown in FIGS. 33A-33C, all activatable antibodies remained stable, and exhibited little aggregation after storage at 50° C. for 7 days, 40° C. for 28 days or freeze-thaw, indicating that these activatable antibodies were very stable under these accelerated stress tests. It is noted that the activatable antibodies had not yet gone through an extensive buffer optimization process, and therefore the stability of the activatable antibodies may be further improved with optimized buffer and excipient.

### G. Effect of Masking Peptide Length on the Masking Efficiency of Activatable Antibodies Targeting HER2

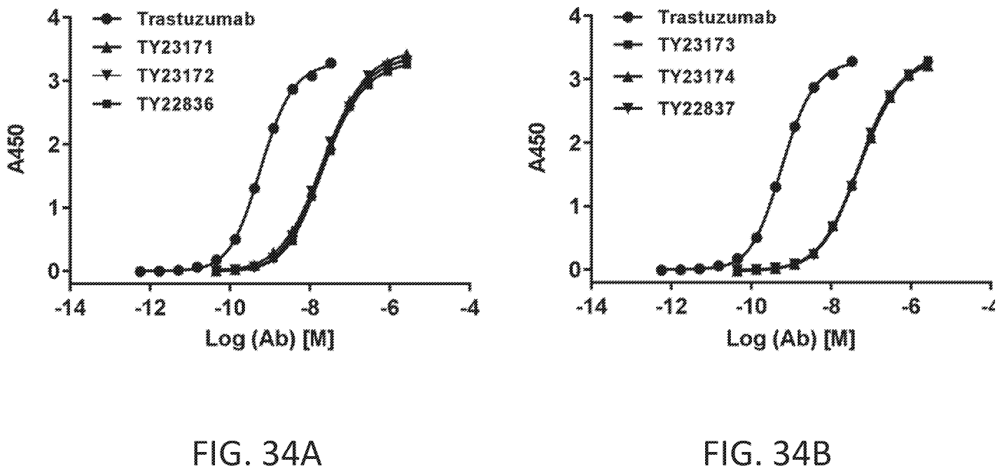

Two of the activatable antibodies (TY22836 and TY22837) were chosen to test the dependence of masking efficiency on the length of masking peptides to suit the specific applications. The masking peptides were shortened from 21 residues to 16 or 14 residues by removing the residues from the N-terminus, leaving only 6 or 4 residues before the first cysteine residue in the masking peptide (Table 18). These activatable antibodies were expressed and purified from mammalian cells and their masking efficiencies were measured as by ELISA as described above and compared to parent antibody trastuzumab. Results from two experiments indicated that these activatable antibodies were made using different masking peptides with lengths ranging from 4 to 11 residues before the first cysteine residue to modulate antibody masking efficiency (FIGS. 34A-34B; Table 18). This seems to suggest that the core masking motif contains the cysteine loop and its immediately adjacent residues, and is sufficient to maintain masking efficiency. In Table 18, the cleavage peptide sequences (SEQ ID NO: 431) are underlined, and the masking efficiency was calculated relative to the parental antibody.

TABLE 18
Masking efficiencies of antibodies with varying masking peptide lengths

| | | | | SEQ ID |
|---|---|---|---|---|
| | | | Masking | NO of |
| | | EC₅₀ | effi- | masking |
| IgG ID | Masking + cleavage peptide sequences (underlined): | (nM) | ciency | peptide |
| TY22836 | EVGSYSANNLACHADPFDCTASGRSAGGGGTPLGLAGSGGS | 23.05 | 38 | 473 |
| TY23171 | EVGNLACHADPFDCTASGRSAGGGGTPLGLAGSGGS | 22.37 | 37 | 474 |
| TY23172 | ENLACHADPFDCTASGRSAGGGGTPLGLAGSGGS | 21.08 | 35 | 475 |
| TY22837 | EVGSYHYASDACDADPFDCQASGRSAGGGGTPLGLAGSGGS | 50.02 | 82 | 469 |
| TY23173 | EVGSDACDADPFDCQASGRSAGGGGTPLGLAGSGGS | 59.62 | 98 | 476 |
| TY23174 | ESDACDADPFDCQASGRSAGGGGTPLGLAGSGGS | 52.57 | 87 | 419 |

### H. Effect of Cleavage Peptide Length on the Masking Efficiency of Activatable Antibodies Targeting HER2

TY23174 was chosen to test the dependence of masking efficiency on the length of the cleavage peptide to suit specific applications. The cleavage peptide of TY23174 was shortened to various lengths (Table 19). Activatable antibodies were expressed and purified from mammalian cells and their masking efficiencies were measured by ELISA as described above and compared to parent antibody trastuzumab.

As shown in Table 19, these activatable antibodies were made using different cleavage peptides with lengths ranging from 4 to 35 residues to modulate antibody masking efficiency. The strong correlation between masking and cleavage motifs was striking: the masking efficiency of TY23941 was enhanced least 30-fold compared to TY23639 when the length of cleavage peptide is truncated from 20 to 4 amino acids. These results indicated that several novel masking peptides can be designed and engineered. In addition, the coupling between masking and cleavage motifs could be further explored. In Table 19, the cleavage peptide sequences are underlined, and masking efficiency was calculated relative to TY23477.

TABLE 19
Relative masking efficiencies of antibodies with varying cleavage peptide lengths

| | | | SEQ ID | SEQ ID |
|---|---|---|---|---|
| | | Relative | NO of | NO of |
| | | masking | masking | cleavage |
| IgG ID | Masking + cleavage peptide sequences (underlined): | efficiency | peptide | peptide |
| TY23946 | ESDACDADPFDCQA | 0.11 | 419 | n/a |
| TY23945 | ESDACDADPFDCQAG | 0.07 | 419 | 477 |
| TY23944 | ESDACDADPEDCQAP | 0.05 | 419 | 478 |
| TY23943 | ESDACDADPFDCQAPL | 0.07 | 419 | 479 |
| TY23942 | ESDACDADPFDCQAPLG | 1.14 | 419 | 480 |
| TY23941 | ESDACDADPFDCQAPLGL | 9.05 | 419 | 481 |
| TY23940 | ESDACDADPFDCQAPLGLA | 7.07 | 419 | 482 |
| TY23694 | ESDACDADPFDCQAPLGLAG | 3.53 | 419 | 128 |
| TY23632 | ESDACDADPFDCQAPLGLAGGS | 1.74 | 419 | 484 |
| TY23477 | ESDACDADPFDCQAPLGLAGSGGS | 1 | 419 | 420 |
| TY23698 | ESDACDADPFDCQAGPLGLAGSGGS | 0.94 | 419 | 484 |
| TY23637 | ESDACDADPFDCQAGGPLGLAGSGGS | 0.66 | 419 | 485 |
| TY23638 | ESDACDADPFDCQAGGGGSPLGLAGSGGS | 0.45 | 419 | 486 |
| TY23639 | ESDACDADPFDCQAGGGGSGGGGSPLGLAGSGGS | 0.3 | 419 | 487 |
| TY23640 | ESDACDADPFDCQAGGGGSGGGGSGGGGSPLGLAGSGGS | 0.22 | 419 | 488 |
| TY23641 | ESDACDADPFDCQAGGGGSGGGGSGGGGSPLGLAGGGGSGSGGS | 0.16 | 419 | 489 |
| TY23642 | ESDACDADPFDCQAGGGGSGGGGSGGGGSPLGLAGGGGSGGGGSGSGGS | 0.14 | 419 | 490 |

### I. Maturation of Masking of Peptide of Activatable Antibodies Targeting HER2

A series of optimized masking peptide sequences of TY23477, and TY23536 were generated, with improved or decreased masking efficiency for different purpose (Tables 20-21). In Tables 20-21, the cleavage peptide sequences are underlined (PLGLAGSGGS, SEQ ID NO: 420 in Table 20, and SGRSAGGGGTPLGLAGSGGS, SEQ ID NO: 431 in Table 21), and masking efficiency was calculated relative to TY23477.

TABLE 20
Optimized masking peptide sequences and masking efficiencies derived from

| | | | | SEQ ID |
|---|---|---|---|---|
| | | FACS | Masking | NO of |
| | Masking + cleavage peptide sequences | EC₅₀ | efficiency | masking |
| IgG ID | (underlined): | (nM) | by FACS | peptide |
| TY23477 | ESDACDADPFDCQAPLGLAGSGGS | | | 419 |
| TY23656 | ESEVCDADPFECQAPLGLAGSGGS | 653.9 | 262 | 491 |
| TY23657 | ESEFCDADPFECQAPLGLAGSGGS | 833.3 | 334 | 492 |
| TY23658 | ESEYCDADPFECQAPLGLAGSGGS | 736.7 | 295 | 493 |
| TY23659 | ESEACDLDPFECQAPLGLAGSGGS | 484.1 | 194 | 494 |
| TY23660 | ESEACDADPFECQFPLGLAGSGGS | 607.5 | 244 | 495 |
| TY23661 | ESEACDADPFECQYPLGLAGSGGS | 353.2 | 142 | 496 |
| TY23662 | ESEVCDLDPFECQFPLGLAGSGGS | 556.3 | 223 | 497 |
| TY23663 | ESDACEADPFDCQAPLGLAGSGGS | 573.1 | 230 | 498 |
| TY23755 | ESDVCDADPFDCQAPLGLAGSGGS | 581.3 | 274 | 499 |
| TY23756 | ESDVCDLDPFDCQAPLGLAGSGGS | 1043 | 492 | 500 |
| TY23757 | ESDVCDLDPFDCQFPLGLAGSGGS | 876.6 | 413 | 501 |
| TY23758 | ESEVCEADPFECQAPLGLAGSGGS | 619.7 | 292 | 502 |
| TY24004 | EVGSYASAPDACDADPYECQAPLGLAGSGGS | ND | ND | 503 |
| TY24005 | EVGSYASVTEACDADPYECSHPLGLAGSGGS | ND | ND | 504 |
| TY24006 | EVGSYSDVPEVCEADPYECQHPLGLAGSGGS | ND | ND | 505 |
| TY24007 | EVGSYHAASEVCDADPYECSHPLGLAGSGGS | ND | ND | 506 |
| TY24008 | EVGSYSDASEVCEADPYECQHPLGLAGSGGS | ND | ND | 507 |
| TY24009 | EVGSYASASEACDLDPYECQVPLGLAGSGGS | ND | ND | 508 |
| TY24010 | EVGSYHDASEVCEADPYECSAPLGLAGSGGS | ND | ND | 509 |
| TY24011 | EVGSYASTPEACEADPYECSVPLGLAGSGGS | ND | ND | 510 |
| TY24012 | EVGSYADASDVCDADPYECTVPLGLAGSGGS | ND | ND | 511 |

TABLE 21
Optimized masking peptide sequences and masking efficiencies derived from

| | | | | SEQ ID |
|---|---|---|---|---|
| | | FACS | Masking | NO of |
| | Masking + cleavage peptide sequences | EC₅₀ | efficiency | masking |
| IgG ID | (underlined): | (nM) | by FACS | peptide |
| TY23536 | EVGSYYNSDDDCASHYDPYTCYYSGRSAGGGGTPLGLAGSGGS | 48.32 | 84 | 460 |
| TY23775 | EVGSYYNSDDDCVSHYDPYTCYYSGRSAGGGGTPLGLAGSGGS | ND | ND | 512 |
| TY23777 | EVGSYYNSDDDCISHYDPYTCYYSGRSAGGGGTPLGLAGSGGS | 104.4 | 181 | 513 |
| TY23780 | EVGSYYNSDDDCASRYDPYTCYYSGRSAGGGGTPLGLAGSGGS | 108.4 | 188 | 514 |
| TY23917 | EVGSYYNSDDDCVSRYDPYTCYYSGRSAGGGGTPLGLAGSGGS | 156 | 271 | 515 |

### J. Maturation of Cleavage Peptide of Activatable Antibodies Targeting HER2

A series of optimized cleavage peptide sequences of TY23477 were generated, with improved cleavage efficiency (Table 22). In Table 22, the cleavage peptide sequences are underlined.

TABLE 22
Masking peptide with modified sequences derived from TY23477

| | | SEQ ID NO | SEQ ID NO |
|---|---|---|---|
| Peptide | Masking + cleavage peptide sequences | of masking | of cleavage |
| ID | (underlined): | peptide | peptide |
| B14555 | ESDACDADPFDCQASAPLGLAAP | 419 | 516 |
| B14537 | ESDACDADPFDCQARPLGLAARM | 419 | 517 |
| B14538 | ESDACDADPFDCQARPLGLASRL | 419 | 518 |
| B14539 | ESDACDADPFDCQAPPLGLASRL | 419 | 519 |
| B14540 | ESDACDADPFDCQAVPLGLAAWK | 419 | 520 |
| B14543 | ESDACDADPFDCQAKPLGLAASL | 419 | 521 |
| B14545 | ESDACDADPFDCQAVPLGLASRL | 419 | 522 |
| B14546 | ESDACDADPFDCQAAPLGLAAKL | 419 | 523 |
| B14547 | ESDACDADPFDCQAAPLGLASWW | 419 | 524 |
| B14549 | ESDACDADPFDCQAIPLGLAARL | 419 | 525 |
| B14550 | ESDACDADPFDCQAAPLGLAVML | 419 | 526 |
| B14552 | ESDACDADPFDCQARPLGLAARL | 419 | 527 |
| B14556 | ESDACDADPFDCQARPLGLAAAL | 419 | 528 |
| B14557 | ESDACDADPFDCQARPLGLAMRL | 419 | 529 |
| B14558 | ESDACDADPFDCQAHPLGLASWR | 419 | 530 |
| B14559 | ESDACDADPFDCQAAPLGLASRM | 419 | 531 |
| B14560 | ESDACDADPFDCQARPLGLAAKL | 419 | 532 |
| B14561 | ESDACDADPFDCQAGWSGRSARP | 419 | 533 |
| B14562 | ESDACDADPFDCQARSSGRSAVW | 419 | 534 |
| B14563 | ESDACDADPFDCQAGGSGRSAKH | 419 | 535 |
| B14564 | ESDACDADPFDCQAPNSGRSAKW | 419 | 536 |
| B14565 | ESDACDADPFDCQAPRSGRSALW | 419 | 537 |
| B14566 | ESDACDADPFDCQATGSGRSAKY | 419 | 538 |
| B14567 | ESDACDADPFDCQAGRSGRSAVW | 419 | 539 |
| B14568 | ESDACDADPFDCQAARSGRSARP | 419 | 540 |
| B14569 | ESDACDADPFDCQAGSGRSARPS | 419 | 541 |
| B14570 | ESDACDADPFDCQARRSGRSAVW | 419 | 542 |
| B14571 | ESDACDADPFDCQAGPSGRSAIY | 419 | 543 |
| B14572 | ESDACDADPFDCQAFRSGRSAVW | 419 | 544 |
| B14573 | ESDACDADPFDCQAHSSGRSARY | 419 | 545 |
| B14574 | ESDACDADPFDCQASNSGRSARY | 419 | 546 |
| B14575 | ESDACDADPFDCQATRSGRSAVW | 419 | 547 |
| B14576 | ESDACDADPFDCQASRSGRSAVW | 419 | 548 |
| B14577 | ESDACDADPFDCQASMSGRSARW | 419 | 549 |
| B14578 | ESDACDADPFDCQARGSGRSAVW | 419 | 550 |
| B14579 | ESDACDADPFDCQASRSGRSARW | 419 | 551 |
| B14580 | ESDACDADPFDCQARGSGRSARH | 419 | 552 |
| B14581 | ESDACDADPFDCQAARSGRSARW | 419 | 553 |
| B14582 | ESDACDADPFDCQASRSGRSAVY | 419 | 554 |
| B14583 | ESDACDADPFDCQARHSGRSAVY | 419 | 555 |

### Example 9. Generation of CD20xCD3 T-Cell-Engaging Bispecific Antibodies

CD20xCD3 bispecific antibodies and activatable versions of CD20xCD3 bispecific antibodies with masked anti-CD3 arms were constructed. The constructs are described in Table 23. Specifically, TY25455 is the parental bispecific antibody of TY25606, and TY25606 has the SAFEbody version on the anti-CD3 arm on HC2. TY25715 is the parental bispecific antibody of TY25716, and TY25716 has the SAFEbody version on the anti-CD3 arm on HC2.

TABLE 23
Amino acid sequences of TY25455, TY25606, TY25715, and TY25716 heavy and light
chains

| | Antibody | | SEQ ID |
|---|---|---|---|
| IgG ID | chain | Amino Acid Sequence | NO |
| TY25455 | LC1 | QIVLSQSPAILSASPGEKVTMTCRASSSVSYIHWFQQKPGKSPK | 564 |
| | | PWIYATSNLASGVPVRFSGSGSGTSYSLTISRVEAEDAATYYCQ | |
| | | QWTSNPPTFGGGTKVEIKRTVAAPSVFIFPPSDEQLKSGTASVV | |
| | | CLLNNFYPREAKVQWKVDNALQSGNSQESVTEQDSKDSTYSLSS | |
| | | TLTLSKADYEKHKVYACEVTHQGLSSPVTKSFNRGEC | |
| TY25455 | HC1 | EVQLVQSGAEVKKPGSSVKVSCKASGYTFTSYNMHWVRQAPGKG | 565 |
| | | LEWIGAIYPGSGDTSYNQKFKGRVTITADKSTSTAYMELSSLRS | |
| | | EDTAVYYCARSTYYGGDWYFNVWGQGTLVTVSSASTKGPSVFPL | |
| | | APSSKSTSGGTAALGCLVKDYFPEPVTVSWNSGALTSGVHTFPA | |
| | | VLQSSGLYSLSSVVTVPSSSLGTQTYICNVNHKPSNTKVDKKVE | |
| | | PKSCDKTHTCPPCPAPELLGGPSVFLFPPKPKDTLMISRTPEVT | |
| | | CVVVDVSHEDPEVKFNWYVDGVEVHNAKTKPREEQYASTYRVVS | |
| | | VLTVLHQDWLNGKEYKCKVSNKALPAPIEKTISKAKGQPREPQV | |
| | | YTLPPSRKKLTKNQVKLTCLVKGFYPSDIAVEWESNGQPENNYK | |
| | | TTPPVLDCDGSFFLYSKLTVDKSRWQQGNVFSCSVMHEALHNHY | |
| | | TQKSLSLSPGK | |
| TY25455 | HC2 | QAVVTQEPSLTVSPGGTVTLTCGSSTGAVTTSNYPNWVQQKPGQ | 566 |
| | | APRGLIGGTNKRAPGVPARFSGSLLGGKAALTLSGAQPEDEAEY | |
| | | YCALWYSNLWVFGGGTKLTVLGGGGSGGGGSGGGGSGGGGSEVQ | |
| | | LVESGGGLVQPGGSLRLSCAASGFTENTYAINWVRQAPGKGLEW | |
| | | VGRIRSKYNNYATYYAESVKGRFTISRDDSKNTLYLQINSLRAE | |
| | | DTAVYYCVRHGNFGTSYVSWFAYWGQGTLVTVSSEPKSSDKTHT | |
| | | CPPCPAPELLGGPSVFLFPPKPKDTLMISRTPEVTCVVVDVSHE | |
| | | DPEVKFNWYVDGVEVHNAKTKPREEQYASTYRVVSVLTVLHQDW | |
| | | LNGKEYKCKVSNKALPAPIEKTISKAKGQPREPQVYTDPPSRDE | |
| | | LTKNQVSLTCLVDGFYPSDIAVEWESNGQPENCYKTTPPVLDSD | |
| | | GSFFLYSKLTVDKSRWQQGNVFSCSVMHEALHNHYTQDSLSLSP | |
| | | GK | |
| TY25606 | LC1 | QIVLSQSPAILSASPGEKVTMTCRASSSVSYIHWFQQKPGKSPK | 564 |
| | | PWIYATSNLASGVPVRFSGSGSGTSYSLTISRVEAEDAATYYCQ | |
| | | QWTSNPPTFGGGTKVEIKRTVAAPSVFIFPPSDEQLKSGTASVV | |
| | | CLLNNFYPREAKVQWKVDNALQSGNSQESVTEQDSKDSTYSLSS | |
| | | TLTLSKADYEKHKVYACEVTHQGLSSPVTKSFNRGEC | |
| TY25606 | HC1 | EVQLVQSGAEVKKPGSSVKVSCKASGYTFTSYNMHWVRQAPGKG | 565 |
| | | LEWIGAIYPGSGDTSYNQKFKGRVTITADKSTSTAYMELSSLRS | |
| | | EDTAVYYCARSTYYGGDWYFNVWGQGTLVTVSSASTKGPSVFPL | |
| | | APSSKSTSGGTAALGCLVKDYFPEPVTVSWNSGALTSGVHTFPA | |
| | | VLQSSGLYSLSSVVTVPSSSLGTQTYICNVNHKPSNTKVDKKVE | |
| | | PKSCDKTHTCPPCPAPELLGGPSVFLFPPKPKDTLMISRTPEVT | |
| | | CVVVDVSHEDPEVKFNWYVDGVEVHNAKTKPREEQYASTYRVVS | |
| | | VLTVLHQDWLNGKEYKCKVSNKALPAPIEKTISKAKGQPREPQV | |
| | | YTLPPSRKKLTKNQVKLTCLVKGFYPSDIAVEWESNGQPENNYK | |
| | | TTPPVLDCDGSFFLYSKLTVDKSRWQQGNVFSCSVMHEALHNHY | |
| | | TQKSLSLSPGK | |
| TY25606 | HC2 | EVGSYPYDDPDCPSHESDCDQGGGPLGLAGGSQAVVTQEPSLTV | 567 |
| | | SPGGTVTLTCGSSTGAVTTSNYPNWVQQKPGQAPRGLIGGTNKR | |
| | | APGVPARFSGSLLGGKAALTLSGAQPEDEAEYYCALWYSNLWVF | |
| | | GGGTKLTVLGGGGSGGGGSGGGGSGGGGSEVQLVESGGGLVQPG | |
| | | GSLRLSCAASGFTENTYAINWVRQAPGKGLEWVGRIRSKYNNYA | |
| | | TYYAESVKGRFTISRDDSKNTLYLQINSLRAEDTAVYYCVRHGN | |
| | | FGTSYVSWFAYWGQGTLVTVSSEPKSSDKTHTCPPCPAPELLGG | |
| | | PSVFLFPPKPKDTLMISRTPEVTCVVVDVSHEDPEVKFNWYVDG | |
| | | VEVHNAKTKPREEQYASTYRVVSVLTVLHQDWLNGKEYKCKVSN | |
| | | KALPAPIEKTISKAKGQPREPQVYTDPPSRDELTKNQVSLTCLV | |
| | | DGFYPSDIAVEWESNGQPENCYKTTPPVLDSDGSFFLYSKLTVD | |
| | | KSRWQQGNVFSCSVMHEALHNHYTQDSLSLSPGK | |
| TY25715 | LC1 | QIVLSQSPAILSASPGEKVTMTCRASSSVSYIHWFQQKPGKSPK | 564 |
| | | PWIYATSNLASGVPVRFSGSGSGTSYSLTISRVEAEDAATYYCQ | |
| | | QWTSNPPTFGGGTKVEIKRTVAAPSVFIFPPSDEQLKSGTASVV | |
| | | CLLNNFYPREAKVQWKVDNALQSGNSQESVTEQDSKDSTYSLSS | |
| | | TLTLSKADYEKHKVYACEVTHQGLSSPVTKSFNRGEC | |
| TY25715 | HC1 | EVQLVQSGAEVKKPGSSVKVSCKASGYTFTSYNMHWVRQAPGKG | 565 |
| | | LEWIGAIYPGSGDTSYNQKFKGRVTITADKSTSTAYMELSSLRS | |
| | | EDTAVYYCARSTYYGGDWYFNVWGQGTLVTVSSASTKGPSVFPL | |
| | | APSSKSTSGGTAALGCLVKDYFPEPVTVSWNSGALTSGVHTFPA | |
| | | VLQSSGLYSLSSVVTVPSSSLGTQTYICNVNHKPSNTKVDKKVE | |
| | | PKSCDKTHTCPPCPAPELLGGPSVFLFPPKPKDTLMISRTPEVT | |
| | | CVVVDVSHEDPEVKFNWYVDGVEVHNAKTKPREEQYASTYRVVS | |
| | | VLTVLHQDWLNGKEYKCKVSNKALPAPIEKTISKAKGQPREPQV | |
| | | YTLPPSRKKLTKNQVKLTCLVKGFYPSDIAVEWESNGQPENNYK | |
| | | TTPPVLDCDGSFFLYSKLTVDKSRWQQGNVFSCSVMHEALHNHY | |
| | | TQKSLSLSPGK | |
| TY25715 | HC2 | EAVVTQEPSLTVSPGGTVTLTCGSSTGAVTTSNYPNWVQQKPGQ | 568 |
| | | APRGLIGGTNKRAPGTPARFSGSLLGGKAALTLSGAQPEDEAEY | |
| | | YCALWYSDLWVFGGGTKLTVLGGGGSGGGGSGGGGSGGGGSEVQ | |
| | | LVESGGGLVQPGGSLRLSCAASGFTFSTYAINWVRQAPGKGLEW | |
| | | VSRIRSKYNNYATYYAESVKDRFTISRDDSKNTLYLQINSLRAE | |
| | | DTAVYYCVRHGNFGTSYVSWFAYWGQGTLVTVSSEPKSSDKTHT | |
| | | CPPCPAPELLGGPSVFLFPPKPKDTLMISRTPEVTCVVVDVSHE | |
| | | DPEVKFNWYVDGVEVHNAKTKPREEQYASTYRVVSVLTVLHQDW | |
| | | LNGKEYKCKVSNKALPAPIEKTISKAKGQPREPQVYTDPPSRDE | |
| | | LTKNQVSLTCLVDGFYPSDIAVEWESNGQPENCYKTTPPVLDSD | |
| | | GSFFLYSKLTVDKSRWQQGNVFSCSVMHEALHNHYTQDSLSLSP | |
| | | GK | |
| TY25716 | LC1 | QIVLSQSPAILSASPGEKVTMTCRASSSVSYIHWFQQKPGKSPK | 564 |
| | | PWIYATSNLASGVPVRFSGSGSGTSYSLTISRVEAEDAATYYCQ | |
| | | QWTSNPPTFGGGTKVEIKRTVAAPSVFIFPPSDEQLKSGTASVV | |
| | | CLLNNFYPREAKVQWKVDNALQSGNSQESVTEQDSKDSTYSLSS | |
| | | TLTLSKADYEKHKVYACEVTHQGLSSPVTKSFNRGEC | |
| TY25716 | HC1 | EVQLVQSGAEVKKPGSSVKVSCKASGYTFTSYNMHWVRQAPGKG | 565 |
| | | LEWIGAIYPGSGDTSYNQKFKGRVTITADKSTSTAYMELSSLRS | |
| | | EDTAVYYCARSTYYGGDWYFNVWGQGTLVTVSSASTKGPSVFPL | |
| | | APSSKSTSGGTAALGCLVKDYFPEPVTVSWNSGALTSGVHTFPA | |
| | | VLQSSGLYSLSSVVTVPSSSLGTQTYICNVNHKPSNTKVDKKVE | |
| | | PKSCDKTHTCPPCPAPELLGGPSVFLFPPKPKDTLMISRTPEVT | |
| | | CVVVDVSHEDPEVKFNWYVDGVEVHNAKTKPREEQYASTYRVVS | |
| | | VLTVLHQDWLNGKEYKCKVSNKALPAPIEKTISKAKGQPREPQV | |
| | | YTLPPSRKKLTKNQVKLTCLVKGFYPSDIAVEWESNGQPENNYK | |
| | | TTPPVLDCDGSFFLYSKLTVDKSRWQQGNVFSCSVMHEALHNHY | |
| | | TQKSLSLSPGK | |
| TY25716 | HC2 | EVGSYPYDDPDCPSHESDCDQGGGPLGLAGGSEAVVTQEPSLTV | 569 |
| | | SPGGTVTLTCGSSTGAVTTSNYPNWVQQKPGQAPRGLIGGTNKR | |
| | | APGTPARFSGSLLGGKAALTLSGAQPEDEAEYYCALWYSDLWVE | |
| | | GGGTKLTVLGGGGSGGGGSGGGGSGGGGSEVQLVESGGGLVQPG | |
| | | GSLRLSCAASGFTFSTYAINWVRQAPGKGLEWVSRIRSKYNNYA | |
| | | TYYAESVKDRFTISRDDSKNTLYLQINSLRAEDTAVYYCVRHGN | |
| | | FGTSYVSWFAYWGQGTLVTVSSEPKSSDKTHTCPPCPAPELLGG | |
| | | PSVFLFPPKPKDTLMISRTPEVTCVVVDVSHEDPEVKFNWYVDG | |
| | | VEVHNAKTKPREEQYASTYRVVSVLTVLHQDWLNGKEYKCKVSN | |
| | | KALPAPIEKTISKAKGQPREPQVYTDPPSRDELTKNQVSLTCLV | |
| | | DGFYPSDIAVEWESNGQPENCYKTTPPVLDSDGSFFLYSKLTVD | |
| | | KSRWQQGNVFSCSVMHEALHNHYTQDSLSLSPGK | |

In addition, the CD20xCD3 bispecific antibodies TAC2415 and TAC2392 were generated. The constructs are described in Table 24. Specifically, TAC2415 is made up of an anti-CD3 LC2 and HC2, and an anti-CD20 LC1 and HC2. TAC2392 is made up of an anti-CD3 HC2, and an anti-CD20 LC1 and HC2.

TABLE 24
Amino acid sequences of TAC2415 and TAC2392 heavy and light chains

| | Antibody | | SEQ ID |
|---|---|---|---|
| IgG ID | chain | Amino Acid Sequence | NO |
| TAC2415 | LC1 | DIQMTQSPSSLSASVGDRVTITCRASSSVSYMHWYQQKPGKAPK | 570 |
| | | PLIYAPSNLASGVPSRFSGSGSGTDFTLTISSLQPEDFATYYCQ | |
| | | QWSFNPPTFGQGTKVEIKRTVAAPSVFIFPPSDEQLKSGTASVV | |
| | | CLLNNFYPREAKVQWKVDNALQSGNSQESVTEQDSKDSTYSLSS | |
| | | TLTLSKADYEKHKVYACEVTHQGLSSPVTKSFNRGEC | |
| TAC2415 | HC1 | EVQLVESGGGLVQPGGSLRLSCAASGYTFTSYNMHWVRQAPGKG | 571 |
| | | LEWVGAIYPGNGDTSYNQKFKGRFTISVDKSKNTLYLQMNSLRA | |
| | | EDTAVYYCARVVYYSNSYWYFDVWGQGTLVTVSSASTKGPSVEP | |
| | | LAPSSKSTSGGTAALGCLVKDYFPEPVTVSWNSGALTSGVHTFP | |
| | | AVLQSSGLYSLSSVVTVPSSSLGTQTYICNVNHKPSNTKVDKKV | |
| | | EPKSCDKTHTCPPCPAPELLGGPSVFLFPPKPKDTLMISRTPEV | |
| | | TCVVVDVSHEDPEVKFNWYVDGVEVHNAKTKPREEQYGSTYRVV | |
| | | SVLTVLHQDWLNGKEYKCKVSNKALPAPIEKTISKAKGQPREPQ | |
| | | VYTLPPSREEMTKNQVSLWCLVKGFYPSDIAVEWESNGQPENNY | |
| | | KTTPPVLDSDGSFFLYSKLTVDKSRWQQGNVFSCSVMHEALHNH | |
| | | YTQKSLSLSPGK | |
| TAC2415 | LC2 | DIVMTQSPDSLAVSLGERATINCKSSQSLLNSRTRKNYLAWYQQ | 572 |
| | | KPGQPPKLLIYWASTRESGVPDRFSGSGSGTDFTLTISSLQAED | |
| | | VAVYYCTQSFILRTFGQGTKVEIKRTVAAPSVFIFPPSDEQLKS | |
| | | GTASVVCLLNNFYPREAKVQWKVDNALQSGNSQESVTEQDSKDS | |
| | | TYSLSSTLTLSKADYEKHKVYACEVTHQGLSSPVTKSFNRGEC | |
| TAC2415 | HC2 | EVQLVQSGAEVKKPGASVKVSCKASGYTFTNYYIHWVRQAPGQG | 573 |
| | | LEWIGWIYPGDGNTKYNEKFKGRATLTADTSTSTAYLELSSLRS | |
| | | EDTAVYYCARDSYSNYYFDYWGQGTLVTVSSASTKGPSVFPLAP | |
| | | SSKSTSGGTAALGCLVKDYFPEPVTVSWNSGALTSGVHTFPAVL | |
| | | QSSGLYSLSSVVTVPSSSLGTQTYICNVNHKPSNTKVDKKVEPK | |
| | | SCDKTHTCPPCPAPELLGGPSVFLFPPKPKDTLMISRTPEVTCV | |
| | | VVDVSHEDPEVKFNWYVDGVEVHNAKTKPREEQYGSTYRVVSVL | |
| | | TVLHQDWLNGKEYKCKVSNKALPAPIEKTISKAKGQPREPQVYT | |
| | | LPPSREEMTKNQVSLSCAVKGFYPSDIAVEWESNGQPENNYKTT | |
| | | PPVLDSDGSFFLVSKLTVDKSRWQQGNVFSCSVMHEALHNHYTQ | |
| | | KSLSLSPGK | |
| TAC2392 | LC1 | QIVLTQSPSSLSASVGDRVTITCRASSSVSYIHWFQQKPGKSPK | 574 |
| | | PLIYATSNLASGVPVRFSGSGSGTDYTLTISSLQPEDFATYYCQ | |
| | | QWTSNPPTFGGGTKVEIKRTVAAPSVFIFPPSDEQLKSGTASVV | |
| | | CLLNNFYPREAKVQWKVDNALQSGNSQESVTEQDSKDSTYSLSS | |
| | | TLTLSKADYEKHKVYACEVTHQGLSSPVTKSFNRGEC | |
| TAC2392 | HC1 | QVQLVQSGAEVKKPGASVKVSCKASGYTFTSYNMHWVRQAPGQG | 575 |
| | | LEWMGAIYPGNGDTSYNQKFQGRVTITADKSISTAYMELSSLRS | |
| | | EDTAVYYCARSTYYGGDWYFNVWGAGTLVTVSSASTKGPSVFPL | |
| | | APSSKSTSGGTAALGCLVKDYFPEPVTVSWNSGALTSGVHTFPA | |
| | | VLQSSGLYSLSSVVTVPSSSLGTQTYICNVNHKPSDTKVDKKVE | |
| | | PKSCDKTHTCPPCPAPPVAGPSVFLFPPKPKDTLMISRTPEVTC | |
| | | VVVDVKHEDPEVKFNWYVDGVEVHNAKTKPREEEYNSTYRVVSV | |
| | | LTVLHQDWLNGKEYKCKVSNKALPAPIEKTISKAKGQPREPQVY | |
| | | TLPPSREEMTKNQVSLTCDVSGFYPSDIAVEWESDGQPENNYKT | |
| | | TPPVLDSDGSFFLYSKLTVDKSRWEQGDVFSCSVMHEALHNHYT | |
| | | QKSLSLSPGK | |
| TAC2392 | HC2 | EVQLVESGGGLVQPGGSLRLSCAASGFTFSTYAMNWVRQAPGKG | 576 |
| | | LEWVGRIRSKYNNYATYYADSVKGRFTISRDDSKNTLYLQMNSL | |
| | | RAEDTAVYYCVRHGNFGDSYVSWFAYWGQGTLVTVSSGKPGSGK | |
| | | PGSGKPGSGKPGSQAVVTQEPSLTVSPGGTVTLTCGSSTGAVTT | |
| | | SNYANWVQQKPGKSPRGLIGGTNKRAPGVPARFSGSLLGGKAAL | |
| | | TISGAQPEDEADYYCALWYSNHWVFGGGTKLTVLEPKSSDKTHT | |
| | | CPPCPAPPVAGPSVFLFPPKPKDTLMISRTPEVTCVVVDVKHED | |
| | | PEVKFNWYVDGVEVHNAKTKPREEQYNSTYRVVSVLTVLHQDWL | |
| | | NGKEYKCKVSNKALPAPIEKTISKAKGQPREPQVYTLPPSREQM | |
| | | TKNQVKLTCLVKGFYPSDIAVEWESNGQPENNYKTTPPVLDSDG | |
| | | SFFLYSKLTVDKSRWQQGNVFSCSVMHEALHNHYTQKSLSLSPG | |
| | | K | |

### Example 10. Administration of Anti-CD3 and Anti-CD20 Bispecific Antibodies to Cynomolgus Monkeys, and In Vitro Preclinical Studies of Anti-CD3 and Anti-CD20 Bispecific Antibodies

The following example describes the results of experiments to evaluate the safety profiles of parental and activatable anti-CD3 and anti-CD20 antibodies in cynomolgus monkeys, as well as in vitro preclinical studies using the parental and activatable anti-CD3 and anti-CD20 antibodies

### Materials and Methods

### Anti-CD3 and Anti-CD20 Bispecific Antibodies

The anti-CD3 and anti-CD20 bispecific antibodies described in Example 9, above, were used.

### Toxicity Study in Cynomolgus Monkeys

A pilot non-GLP toxicology and pharmacology study was performed in male cynomolgus monkeys (Macaca fascicularis) to determine the ability of the CD3xCD20 bispecific antibodies to deplete B-cell populations in these animals. Each animal received either a parental bispecific antibody or SAFEbody/bispecific antibody with four dosages (0.3, 1, 3, 10 mg/kg), as summarized in Table 25, below. Blood was drawn immediately prior to dosing in order to establish baseline levels for B and T cells in these animals. TY25455, TY25715and TY25716 were administered by i.v. infusion and blood was drawn at 8 hours, and 1, 7, 14, and 21 days post dosing. Following day 21 post-dose, blood was drawn every one week until the conclusion of the study. TY25606 was administered by i.v. infusion and blood was drawn at 8 hours, and 1, 3, 7, 10, 14, and 27 days post dosing. Following day 27 post-dose, blood was drawn every one week until the conclusion of the study. Blood samples were analyzed by FACS for B and T cell markers and the absolute number of these cell types was determined. Serum samples were also analyzed for cytokine levels (IFNγ, IL-2 and other cytokines) using standard analytic methods.

TABLE 25
CD20 × CD3 Parental and SAFE Bispecific
Antibody Study Design in Cyno Monkeys

| | | | | 1^{st} | 2^{nd} | 3^{rd} | 4^{th} |
|---|---|---|---|---|---|---|---|
| Antibody | Cynomolgus | Dosing | Dosing | Dosage | Dosage | Dosage | Dosage |
| treatment | monkey | schedule | route | (mg/kg) | (mg/kg) | (mg/kg) | (mg/kg) |
| TY25455 | 1 male | 4 doses | i.v. | 0.3 | 1 | 3 | 10 |
| (parental) | | | | | | | |
| TY25606 | 1 male | 4 doses | i.v. | 0.3 | 1 | 3 | 10 |
| (SAFEbody) | | | | | | | |
| TY25715 | 1 male | 4 doses | i.v. | 0.3 | 1 | 3 | 10 |
| (parental) | | | | | | | |
| TY25716 | 1 male | 4 doses | i.v. | 0.3 | 1 | 3 | 10 |
| (SAFEbody) | | | | | | | |

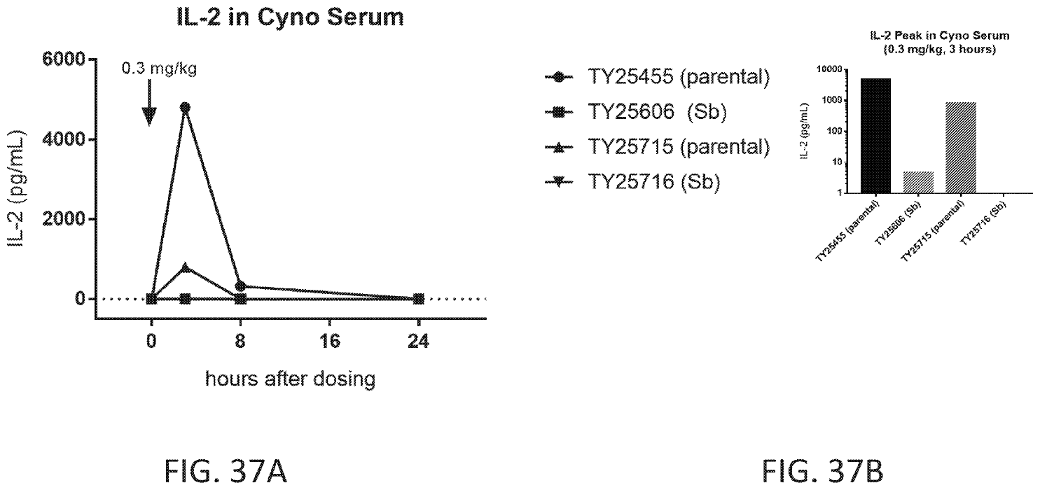

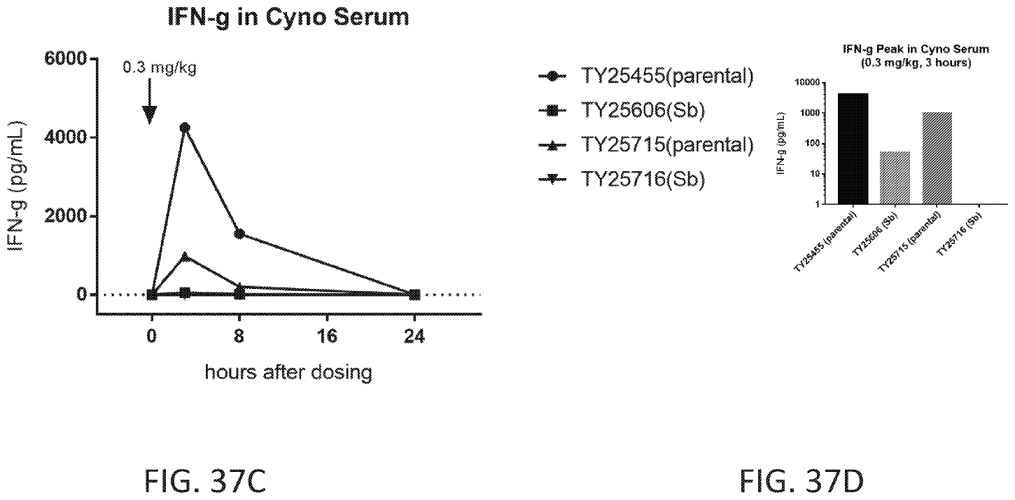

Monkey IL-2 in serum was detected by using the ELISA kit (U-CyTech, CT142A), following the manufacture's instrument (see FIGS. 37A-37B). 96-well ELISA plates were coated with capture antibody overnight at 4° C. After washing with 0.05% Tween 20 in PBS (PBST) 3 times, the plates were blocked with 3% milk in PBS. Diluted plasma samples were added to the ELISA plate and incubated for 1 hour at 37° C. The plates were then washed with PBST and incubated with biotynilated detection antibody for 1 hour at 37° C. After washing with PBST, the plates were further incubated with streptavidin-HRP, developed by adding TMB, and the reaction was stopped by adding the stop solution. The absorbance at 450 nm was measured by micro-plate reader. The data was fit in 4 parameter regression mode on Graphpad Prism.

Monkey IFN-γ in serum was detected by using the ELISA kit (U-CyTech, CT141A), following the manufacture's instrument (see FIGS. 37C-37D). 96-well ELISA plates were coated with capture antibody overnight at 4° C. After washing with 0.05% Tween 20 in PBS (PBST) for 3 times, the plates were blocked with 3% milk in PBS. Diluted plasma samples were added to the ELISA plate and incubated for 1 hour at 37° C. The plates were then washed with PBST and incubated with biotynilated detection antibody for 1 hour at 37° C. After washing with PBST, the plates were further incubated with streptavidin-HRP, developed by adding TMB, and the reaction was stopped by adding the stop solution. The absorbance at 450 nm was measured by micro-plate reader. The data was fit in 4 parameter regression mode on Graphpad Prism.

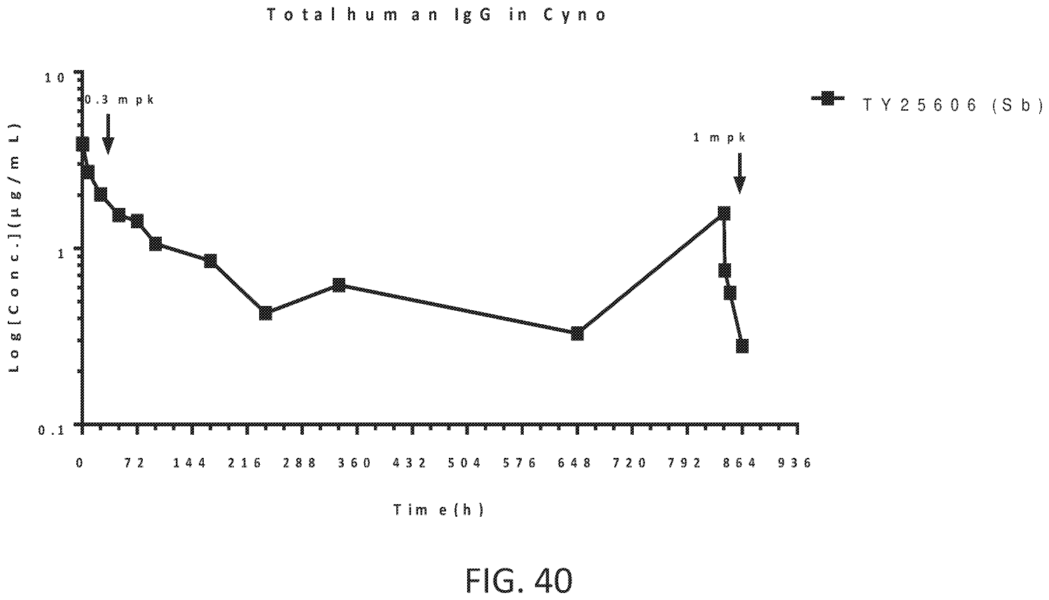

The level of total human IgG in the treated cynomolgus monkeys was measured using FACS (see FIG. 40). 96-well ELISA plates were coated with goat Anti-hIgG at a concentration of 2 μg/mL overnight at 4° C. After washing with 0.05% Tween 20 in PBS (PBST) 3 times, the plates were blocked with 3% milk in PBS. Diluted plasma samples were added to the ELISA plate and incubated for 1 hour at 37° C. The plates were then washed with PBST and incubated with anti-human IgG (Fab Specific)-Peroxidase antibody for 1 hour at 37° C. After washing with PBST, the plates were developed by adding TMB, and the reaction was stopped by adding phosphoric acid. The absorbance at 450 nm was measured by micro-plate reader. The data was fit in 4 parameter regression mode on Graphpad Prism.

Further, pharmacodynamics parameters (including absolute T and B cell counts, and measurements of CD69 expression on CD4+ and CD8+ T cells) were measured by FACS, and pharmacokinetic parameters were measured by ELISA. Blood chemistry tests including measurements of AST/ALT/ALP and bilirubin were performed. Routine blood tests, routine urine tests, electrocardiograms and measurements of blood pressure were performed as well. Finally, pathology analyses were performed, including gross anatomy and critical organ weighing, and HE staining for heart, liver, kidney, spleen, and lung.

### In Vitro Preclinical Studies

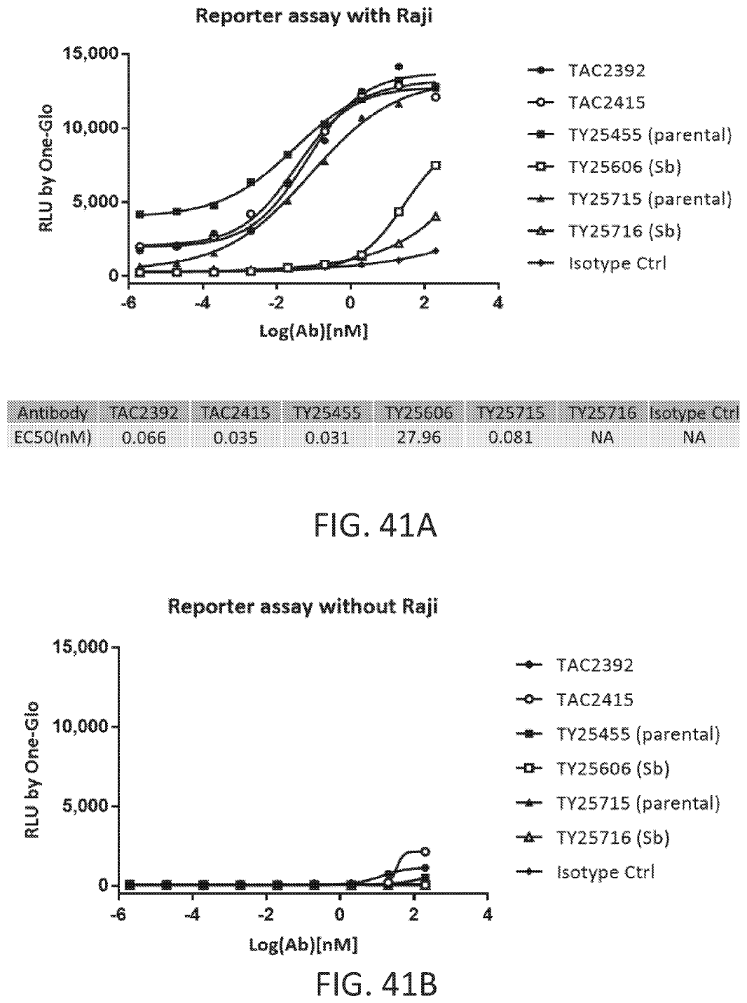

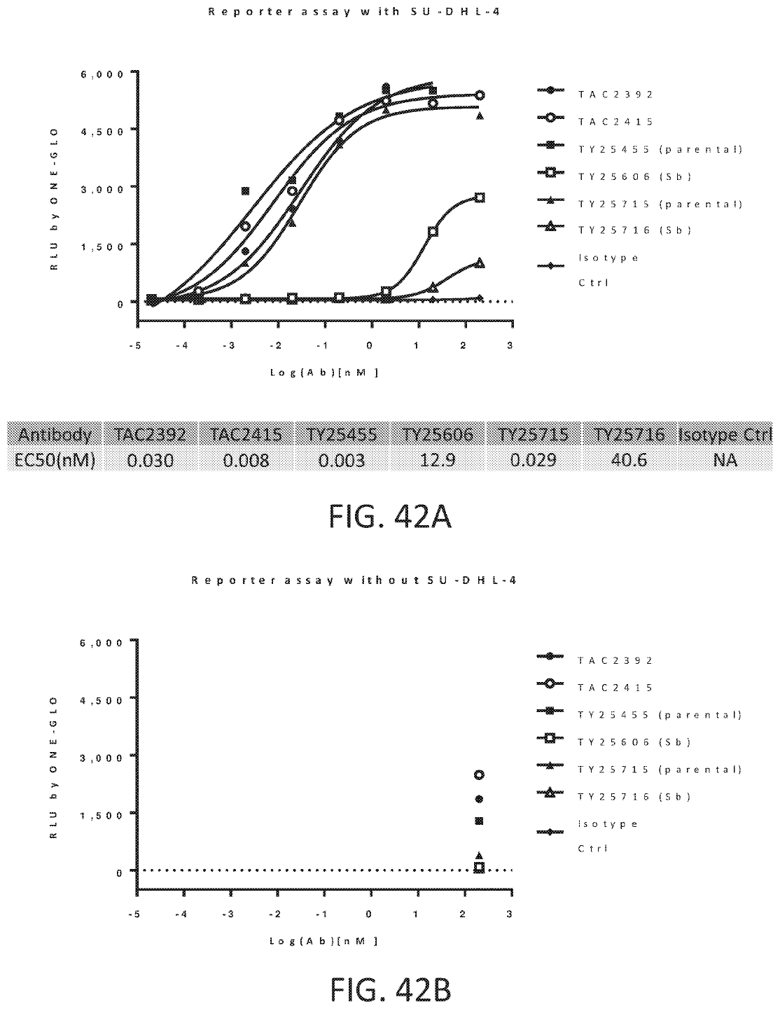

The effect of parental or activatable anti-CD3 and anti-CD20 bispecific antibodies on reporter assays with or without Raji or SU-DHL-4 tumor cells was measured (FIGS. 41A-41B, and 42A-42B). Specifically, the T cell activation activity of the bispecific antibodies was evaluated with Jurkat-NFAT-Luc luciferase reporter assay in the presence or absence of Raji cells (FIGS. 41A-41B). Briefly, Raji cells were seeded in 96-well plates at 2.0×10⁴ per well and incubated with serial diluted test antibodies for 30 minutes at 37° C. in 1% FBS/1640 buffer. After that, Jurkat-NFAT-Luc effector cells were added at 1.0×10⁵ cells/well (E:T ratio, 5:1) and incubated for another 6 hours. Groups without Raji cells were set as no target cells control. The plates were read with One-Glo luciferase reagent using a Multi-mode Plate Reader (Molecular Devices), and data was analyzed and fitted with four-parameter non-linear regression to get EC50 values by GraphPad Prism software.

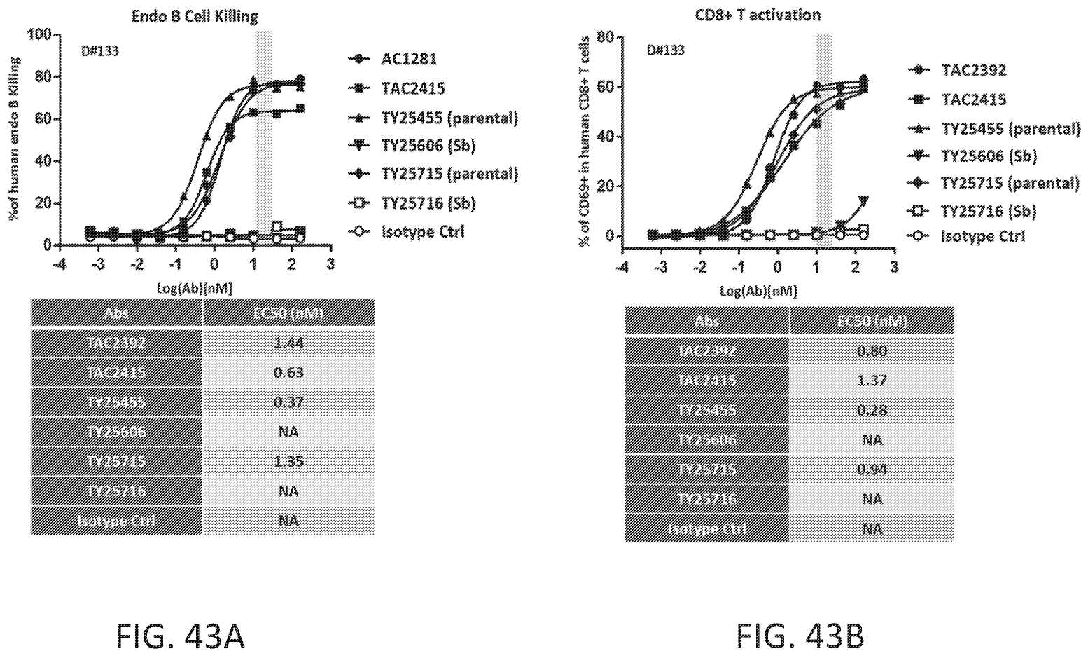

Further, the effect of parental or activatable anti-CD3 and anti-CD20 bispecific antibodies on B cell killing and CD8+ T cell activation was measured in comparison to TAC2392, TAC2415, and an isotype control (FIGS. 43A-43B). Specifically, for endogenous B cell killing assays, human PBMCs were freshly purified with Ficoll gradient solution—Histopaque 1077 (Sigma) from healthy donor. After washing, 2×10⁵ PBMC cells were incubated with serial diluted test antibodies for 24 hours. Following that, cells were washed and stained Live/Dead reagent FVS-700 for 10 minutes at 4° C. Cells were washed and stained with T or B cell surface markers (PerCP-Cy5.5 Anti-Human CD4, BV510 anti-human CD8, APC-Cy7 Mouse Anti-Human CD3, FITC anti-human CD69 and PE-Cy7 Mouse Anti-Human CD19) for 30 minutes at 4° C. After washing, cells were analyzed by FACS (CytoFlx, BeckMan) and dead B cells were gated out as CD19+ FVS+ cells. Activated T cells were gated as CD8+CD69+ T cells. Percentage of CD19+ FVS+ cells or CD8+CD69+ T cells were fitted with antibody concentrations using 4-parameter four-parameter non-linear regression to get EC₅₀ values by GraphPad Prism software.

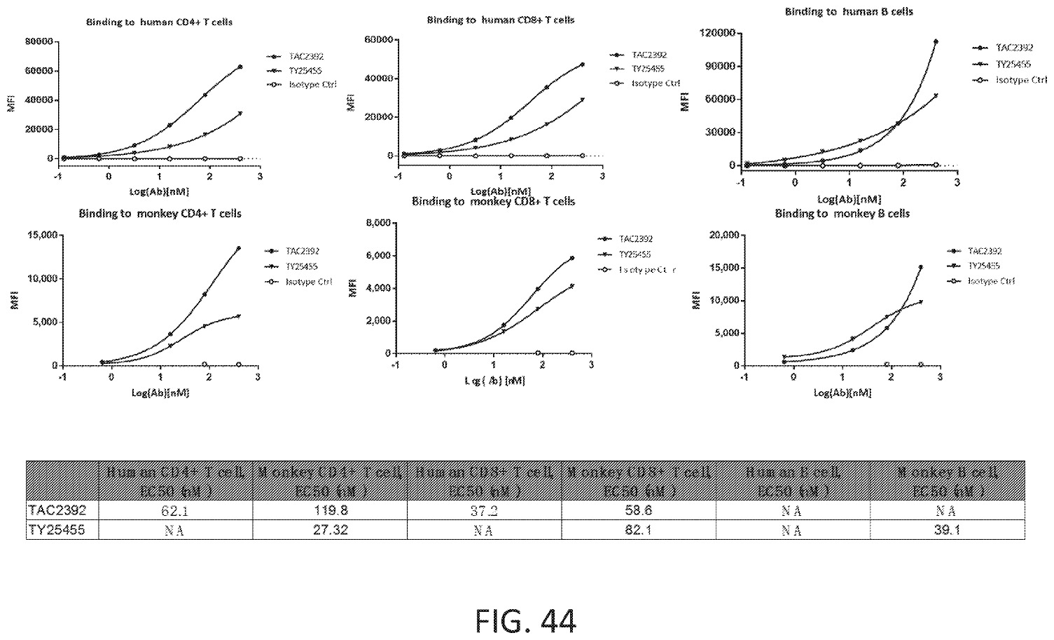

In addition, T and B cell binding by TY25455, TAC2392, and an isotype control was measured (FIG. 44). Binding of CD20xCD3 bispecific or SAFE-bispecific antibodies to purified human CD3+ T cells was determined by flow cytometry. Briefly, human or monkey PBMC were freshly purified with Ficoll gradient solution—Histopaque 1077 (Sigma) from a healthy donor and CD3+ T cells were isolated using EasySep™ Human T cell Enrichment Kit (StemCell). Before staining, 1.4×10⁷ human Fc receptors on CD3+ T cells were blocked with Human Fc Block (BD). Then cells were incubated with fluorescence labeled antibody mixture (APC-Cy7 Mouse Anti-human CD3, PerCP-CyTM5.5 Anti-human CD4 and FITC anti-human CD8) for 15 minutes at 4° C. After washing the cells once with PBS supplemented with 2% FBS, cells were aliquoted to 96-well plates at 2×10⁵ cells per well, and cells were incubated with serial diluted bispecific, SAFE-bispecific or IgG1 isotype control antibodies for 30 minutes at 4° C. After washing the cells twice with PBS supplemented with 2% FBS, cell surface bound antibody was detected by incubating the cells with APC-labeled mouse anti-human IgG Fc secondary antibody for 30 minutes at 4° C. Cells were washed and detected by FACS using Cytoflex (BeckMan) and data was analyzed by FlowJo. The mean fluorescence (MFI) of each antibody binding to human or monkey CD4+ T cells and CD8+ T cell populations were fitted with four-parameter non-linear regression to get EC₅₀ values by GraphPad Prism software.

Binding of CD20xCD3 bispecific or SAFE-bispecific antibodies to purified human B cells was determined by flow cytometry. Briefly, human or monkey PBMC were freshly purified with Ficoll gradient solution—Histopaque 1077 (Sigma) from a healthy donor and B cells were isolated using EasySep™ Human B Cell Isolation Kit (StemCell). The purified B cells were incubated with PE/Cy7 Anti-human CD19 for 30 minutes at 4° C. After washing the cells with PBS supplemented with 2% FBS, cells were aliquoted to 96-well plates at 1×10⁵ cells per well, and cells were incubated with serial diluted bispecific, SAFE-bispecific or IgG1 isotype control antibodies for 30 minutes at 4° C. After washing the cells twice with PBS supplemented with 2% FBS, cell surface bound antibody was detected by incubating the cells with APC-labeled mouse anti-human IgG Fc secondary antibody for 30 minutes at 4° C. Cells were washed and detected by FACS using Cytoflex (BeckMan) and data was analyzed by FlowJo. The mean fluorescence (MFI) of each antibody binding to human B cells (CD19+) were fitted with four-parameter non-linear regression to get EC₅₀ values by GraphPad Prism software.

### Results

Results in Cynomolgus Monkeys Following Administration Of First Dose (0.3 mg/Kg)

TY25455, TY25606, TY25715, or TY25716 were administered to cynomolgus monkeys, and cytokine release assays were performed (see FIGS. 37A-37D). A significant cytokine storm was observed after TY25455 and TY25715 dosing at 0.3 mg/kg. No obvious cytokine release was observed after TY25606 and TY25716 dosing at 0.3 mg/kg.

The behavior of the monkeys was monitored following antibody administration, as shown in Table 26, below.

TABLE 26
Abnormal Behavior Report in Cyno Monkeys Administered
CD20 × CD3 Parental or SAFE Bispecific Antibodies

| Antibody treatment | Abnormal behavior report |
|---|---|
| TY25455 (parental) | Vomit ~1 hour after 0.3 mg/kg dosage |
| | on day 1. Subsequently recovered. |
| TY25606 (SAFEbody) | Normal. |
| TY25715 (parental) | Vomit ~1 hour after 0.3 mg/kg dosage |
| | on day 1. Subsequently recovered. |
| TY25716 (SAFEbody) | Normal. |

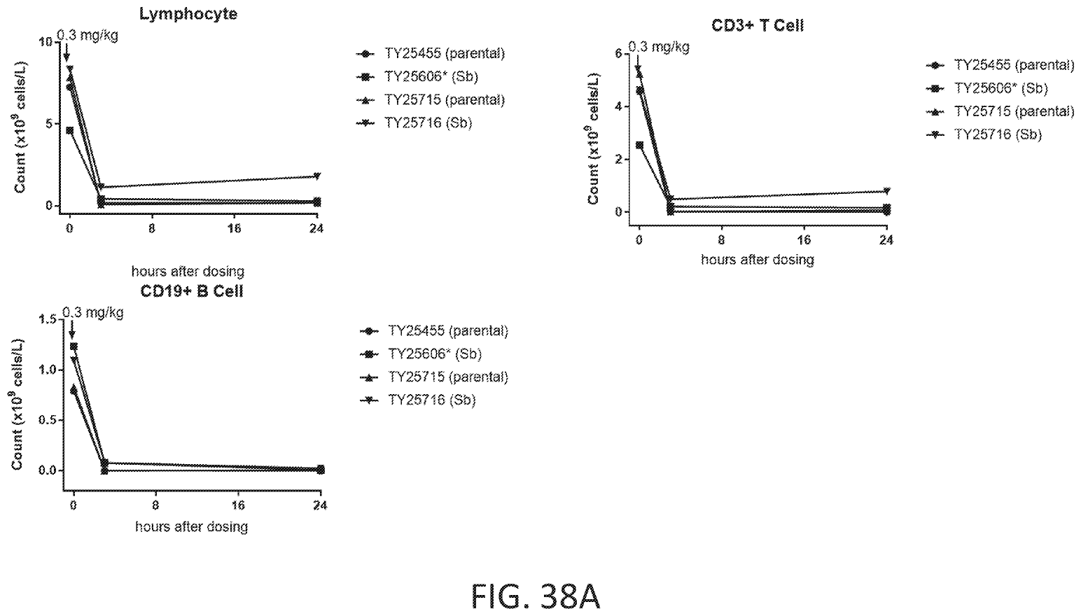

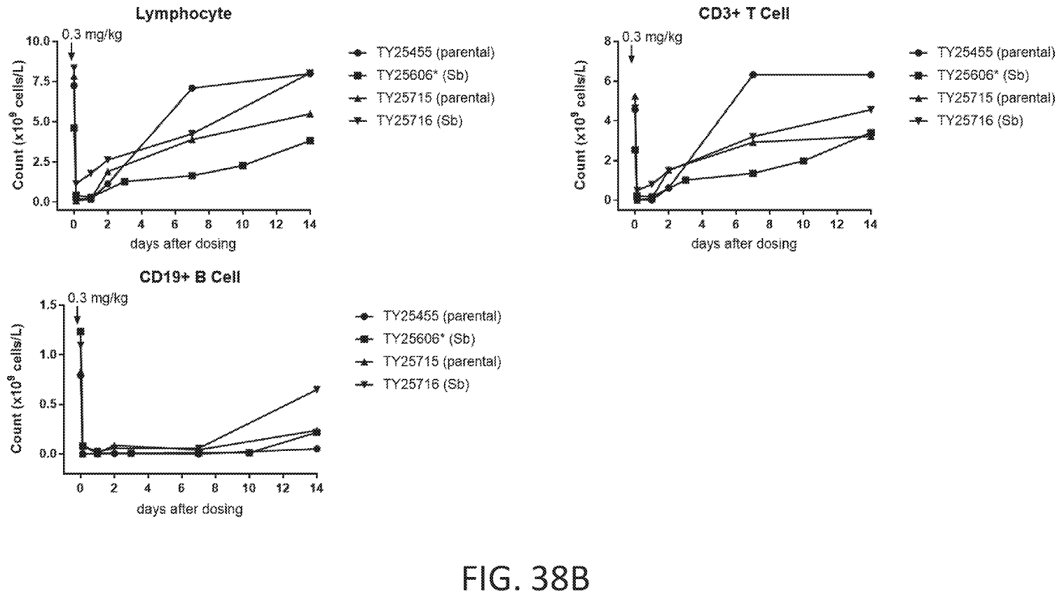

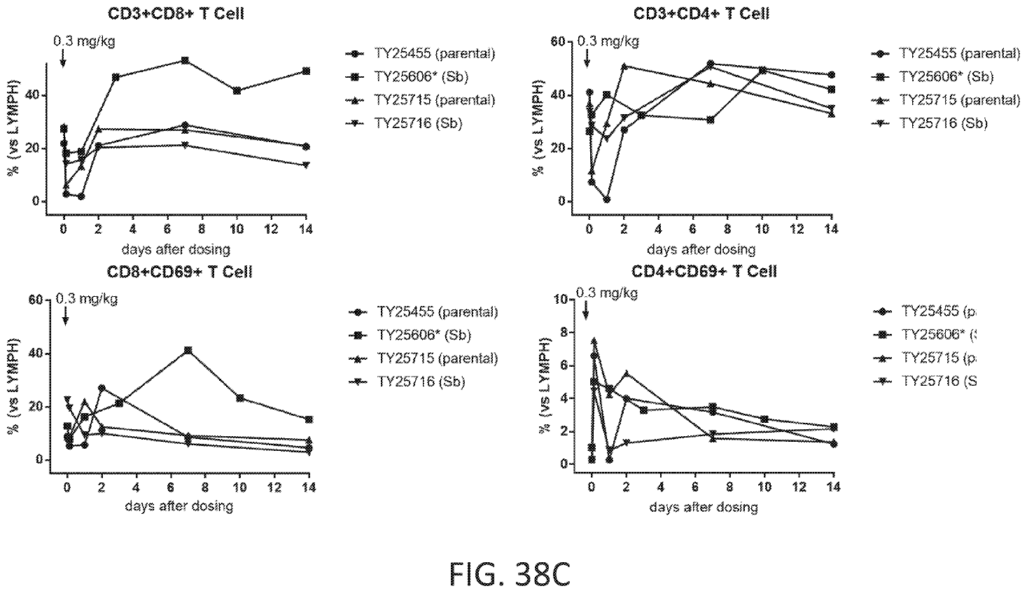

PD markers were measured by FACS (see FIGS. 38A-38C). T cell transmigration and B cell depletion were observed 3 hours after antibody injection (both with parental and SAFEbody antibodies, 0.3 mg/kg).

Results in Cynomolgus Monkeys Following Administration of Second Dose (1 mg/Kg)

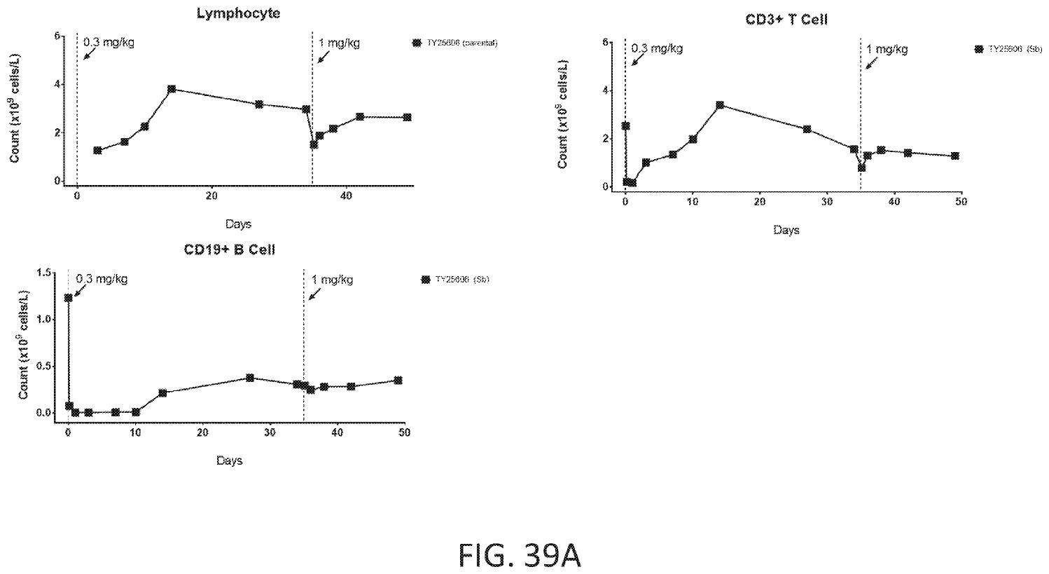

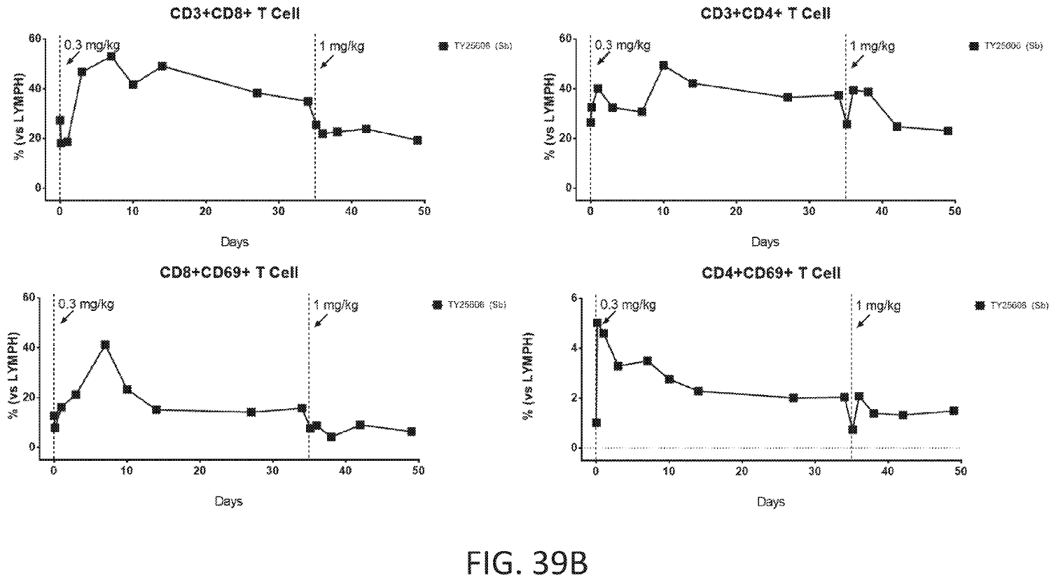

T cell transmigration and B cell depletion were observed 3 hours after antibody injection (both parental and SAFEbody, 0.3 mg/kg). However, the second dosing (1 mg/kg) did not induce significant T cell transmigration and B cell depletion (FIGS. 39A-39B).

TY25606 showed a normal PK curve in the first dosage (0.3 mg/kg). However, the peak concentration was very low in the second dosage (1 mg/kg) and the Ab was almost non-detectable 24 hours after dosing, indicating the existence of ADA (FIG. 40).

### In Vitro Preclinical Studies

The effect of parental or activatable anti-CD3 and anti-CD20 bispecific antibodies on a reporter assay with or without Raji tumor cells was measured (FIGS. 41A-41B). TY25455 showed higher activity than TY25715 in the reporter assay using Raji tumor cells.

The effect of parental or activatable anti-CD3 and anti-CD20 bispecific antibodies on a reporter assay with or without SU-DHL-4 tumor cells was measured (FIGS. 42A-42B). TY25455 showed higher activity than TY25715 in the reporter assay using SU-DHL-4 tumor cells.

The effect of parental or activatable anti-CD3 and anti-CD20 bispecific antibodies on an in vitro B cell killing assay was measured. TY25455 promoted B cell killing and CD8+ T cell activation (FIGS. 43A-43B).

T and B cell binding was measured using FACS (FIG. 44). TY25455 bound to human and monkey T and B cells with similar affinities.

### Example 11: Generation and Characterization of Additional CD20xCD3 Bispecific Antibodies

### A. Generation of the CD20xCD3 Bispecific Antibodies

Bispecific antibodies comprising an anti-CD3-specific binding domain and an anti-CD20-specific binding domain were constructed using standard methodologies. The anti-CD3-specific domain comprises a single-chain variable fragment (“CD3-scFv”) wherein the variable regions of the heavy and light chains are connected by a short linker peptide. The anti-CD20-specific domain comprises a heavy chain variable region (“CD20-VH”) paired with a light chain variable region (“CD20-VL”). Several different CD3-scFv, CD20-VH and CD20-VL components were used in the different bispecific antibodies of the following example.

A summary of the component parts of the antigen-binding domains of the various bispecific antibodies made in accordance with this example is shown in Table 27.

TABLE 27
Construction of bispecific antibodies

| BsAb ID | CD3-scFv arm | CD20 VH arm | CD20 VL arm |
|---|---|---|---|
| TY25715 | TY24742 | VH-13 | VL-21 |
| TY26619 | TY24742 | VH-1 | VL-2 |
| TY26620 | TY24742 | VH-1 | VL-13 |
| TY26621 | TY24742 | VH-1 | VL-15 |
| TY26622 | TY24742 | VH-13 | VL-2 |
| TY26623 | TY24742 | VH-13 | VL-13 |
| TY26624 | TY24742 | VH-13 | VL-15 |
| TY26625 | TY24742 | VH-21 | VL-2 |
| TY26626 | TY24742 | VH-21 | VL-13 |
| TY26627 | TY24742 | VH-21 | VL-15 |
| TY26628 | TY24742_Mut-2 | VH-1 | VL-2 |
| TY26629 | TY24742_Mut-2 | VH-1 | VL-13 |
| TY26630 | TY24742_Mut-2 | VH-1 | VL-15 |
| TY26631 | TY24742_Mut-2 | VH-13 | VL-2 |
| TY26632 | TY24742_Mut-2 | VH-13 | VL-13 |
| TY26633 | TY24742_Mut-2 | VH-13 | VL-15 |
| TY25455 | TY24742_Mut-2 | VH-13 | VL-21 |
| TY26634 | TY24742_Mut-2 | VH-21 | VL-2 |
| TY26635 | TY24742_Mut-2 | VH-21 | VL-13 |
| TY26636 | TY24742_Mut-2 | VH-21 | VL-15 |

The CDRs of the anti-CD3 domains (TY24742 and TY24742-mut2) of the bispecific antibodies are shown in Table 7.

The amino acid sequence identifiers for the various heavy chain variable regions and light chain variable regions of the anti-CD20 domains, and their corresponding CDRs of the anti-CD20 domains of the bispecific antibodies were shown in Tables 28-31.

TABLE 28
Amino acid sequence of heavy chain CDRs of the anti-CD20 arm

| CD20- | | | |
|---|---|---|---|
| VH ID | HCDR1 | HCDR2 | HCDR3 |
| VH-1 | SYNIH | AIYPGSGDTSYNQKFKG | STYYGGDWYFNV |
| | (SEQ ID NO: 86) | (SEQ ID NO: 557) | (SEQ ID NO: 558) |
| VH-13 | SYNMH | AIYPGSGDTSYNQKFKG | STYYGGDWYFNV |
| VH-21 | (SEQ ID NO: 556) | (SEQ ID NO: 557) | (SEQ ID NO: 558) |

TABLE 29
Amino acid sequence of heavy chain CDRs of the anti-CD20 arm

| CD20- | | SEQ ID |
|---|---|---|
| VH ID | Amino Acid Sequence | NO |
| VH-1 | EVQLVQSGAEVKKPGSSVKVSCKASGYTFTSYNIHWVRQAPGKGLEWIGAIY | 87 |
| | PGSGDTSYNQKFKGRVTITADKSTSTAYMELSSLRSEDTAVYYCARSTYYGG | |
| | DWYFNVWGQGTLVTVSS | |
| VH-13 | EVQLVQSGAEVKKPGSSVKVSCKASGYTFTSYNMHWVRQAPGKGLEWIGAIY | 562 |
| | PGSGDTSYNQKFKGRVTITADKSTSTAYMELSSLRSEDTAVYYCARSTYYGG | |
| | DWYFNVWGQGTLVTVSS | |
| VH-21 | EVQLVQSGAEVKKPGSSVKVSCKASGYTFTSYNMHWVRQAPGQGLEWIGAIY | 88 |
| | PGSGDTSYNQKFKGRVTITADKSISTAYMELSSLRSEDTAVYYCARSTYYGG | |
| | DWYFNVWGQGTLVTVSS | |

TABLE 30
Amino acid sequence of light chain CDRs of the anti-CD20 arm

| CD20- | | | |
|---|---|---|---|
| VL ID | LCDR1 | LCDR2 | LCDR3 |
| VL-2 | RASSSVSYIH | ATSNLAS | QQWTSNPPT |
| | (SEQ ID NO: 559) | (SEQ ID NO: 560) | (SEQ ID NO: 561) |
| VL-13 | RASSSVSYIH | ATSNLAS | QQWTSNPPT |
| | (SEQ ID NO: 559) | (SEQ ID NO: 560) | (SEQ ID NO: 561) |
| VL-15 | RASSSVSYIH | ATSNLAS | QQWTSNPPT |
| | (SEQ ID NO: 559) | (SEQ ID NO: 560) | (SEQ ID NO: 561) |
| VL-21 | RASSSVSYIH | ATSNLAS | QQWTSNPPT |
| | (SEQ ID NO: 559) | (SEQ ID NO: 560) | (SEQ ID NO: 561) |

TABLE 31
Amino acid sequence of anti-CD20 arm light chain variable

| CD20- | | SEQ ID |
|---|---|---|
| VL ID | Amino Acid Sequence | NO |
| VL-2 | DIVLTQSPSSLSASVGDRVTITCRASSSVSYIHWYQQKPGKSPKPLIYATSN | 89 |
| | LASGVPVRFSGSGSGTDYTLTISSLQPEDFATYYCQQWTSNPPTFGQGTKVE | |
| | IKR | |
| VL-13 | DIQLTQSPSSLSASVGDRVTITCRASSSVSYIHWFQQKPGKAPKLLIYATSN | 90 |
| | LASGVPSRFSGSGSGTDFTLTISSLQPEDFATYYCQQWTSNPPTFGQGTKVE | |
| | IKR | |
| VL-15 | DIQLTQSPSSLSASVGDRVTITCRASSSVSYIHWYQQKPGKAPKPLIYATSN | 91 |
| | LASGVPSRFSGSGSGTDFTLTISSLQPEDFATYYCQQWTSNPPTFGQGTKVE | |
| | IKR | |
| VL-21 | QIVLSQSPAILSASPGEKVTMTCRASSSVSYIHWFQQKPGKSPKPWIYATSN | 92 |
| | LASGVPVRFSGSGSGTSYSLTISRVEAEDAATYYCQQWTSNPPTFGGGTKVE | |
| | IKR | |

### B. Screening of the CD20xCD3 Bispecific Antibodies

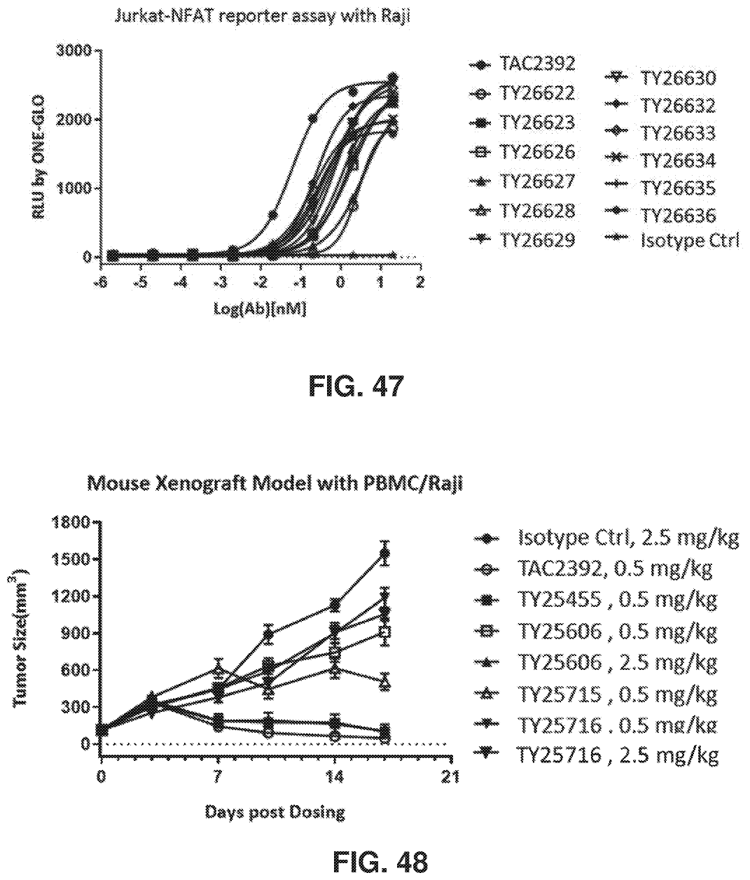

The CD20xCD3 bispecific antibodies were screened using the luciferase-based reporter assay. The Jurkat/NFAT-Luc cells (1×10⁵ cells/well), which contains a luciferase gene under the control of the NFAT response element, were mixed with CD20+ Raji cells (2×10⁴ cells/well) at the E/T ratio of 5:1 in the presence of the CD20xCD3 bispecific antibodies with serial dilution. After incubation for 5 hours at 37° C. and 5% C02, ONE-GLO was added and the luminescence was measured by a multiplate reader. As shown in FIG. 47, the bispecific antibodies activate luciferase in a dose-dependent manner.

### C. Stability

Antibody stabilities were examined under different stress conditions. As shown in Table 32, TY25606 of 1 mg/mL remained stable after 6 cycles of freezing (−80° C.) and thawing (room temperature), as analyzed by SEC-HPLC. As shown in Table 33, TY25716 of 1 mg/mL showed 0.7% decrease of SEC main peak after 6 cycles of freeze and thaw. After fourteen days at 4 or 40° C., there was little change of HMW aggregate or low molecular weight (LMW) fragments for both molecules. TY25606 and TY25716 were also stable under pH 3.8 for 3 hours and pH 8.0 for 24 hours at room temperature. Taken together, these results indicate that even without formulation optimization, TY25606 and TY25716 had excellent developability profile.

TABLE 32
changes of HMW % in SEC-HPLC analysis under different stress conditions for TY25606

| | No-treatment | | | After-treatment | | |
|---|---|---|---|---|---|---|
| | | Main | | | Main | |
| Conditions | HMW % | peak % | LMW % | HMW % | peak % | LMW % |
| Freeze thaw 6 cycles | 5.9% | 94.1% | 0% | 5.0% | 95% | 0% |
| Storage stability at 4° C. for 14 | 5.4% | 94.6% | 0% | 5.5% | 94.5% | 0% |
| days | | | | | | |
| Storage stability at 40° C. for 14 | 5.4% | 94.6% | 0% | 5.1% | 94.4% | 0.5% |
| days | | | | | | |
| pH stability at pH 3.8 for 3 | 6.0% | 94.0% | 0% | 5.9% | 94.1% | 0% |
| hours at R.T. | | | | | | |
| pH stability at pH 8.0 for 24 | 6.0% | 94.0% | 0% | 5.7% | 94.3% | 0% |
| hours at R.T. | | | | | | |

TABLE 33
changes of HMW % in SEC-HPLC analysis under different stress conditions for TY25716

| | No-treatment | | | After-treatment | | |
|---|---|---|---|---|---|---|
| | | Main | | | Main | |
| Conditions | HMW % | peak % | LMW % | HMW % | peak % | LMW % |
| Freeze thaw 6 cycles | 1.7% | 98.2% | 0.1% | 2.5% | 97.5% | 0% |
| Storage stability at 4° C. for 14 | 1.7% | 98.2% | 0.1% | 1.7% | 98.3% | 0% |
| days | | | | | | |
| Storage stability at 40° C. for 14 | 1.7% | 98.2% | 0.1% | 2.5% | 96.4% | 1.1% |
| days | | | | | | |
| pH stability at pH 3.8 for 3 | 1.3% | 98.7% | 0% | 1.1% | 98.8% | 0.1% |
| hours at R.T. | | | | | | |
| pH stability at pH 8.0 for 24 | 1.3% | 98.6% | 0% | 1.7% | 98.2% | 0.1% |
| hours at R.T. | | | | | | |

### D. Binding to Human and Monkey CD3+ T Cells

The abilities of the SAFEbody/bispecific antibodies to bind to human and monkey CD3+ T cells were tested by flow cytometry. Human PBMCs were freshly isolated from the blood of healthy donors by density gradient centrifugation using Histopaque-1077 (Sigma). Human CD3+ T cells were further isolated from PBMCs using the Human T Cell Enrichment Kit (Stem Cell Technologies). Monkey PBMCs were isolated from a naïve cynomolgus monkey by density gradient centrifugation using Histopaque-1077 (Sigma). Monkey CD3+ T cells were isolated from PBMCs using the Pan T Cell Isolation Kit Non-Human Primate (Miltenyi Biotec). Purified human or monkey CD3+ T cells were stained with the testing antibodies as the primary antibody and the APC-labelled anti-human-Fc antibody (BioLegend) as the secondary antibody. After washing, the cells were analyzed on the CytoFLEX flow cytometer (Beckman Coulter) and the data was analyzed by FlowJo software.

As shown in Table 34, TY25455 (parental antibody) and MMP-9 cleaved TY25606 (Sb/Bs antibody) showed comparable binding affinity to human and monkey CD3+ T cells, while TY25606 and control IgG showed no binding.

TABLE 34
summary of antibody binding to human and monkey T and B cells

| | Human CD3+ | | Monkey CD3+ | | Human B | | Monkey B | |
|---|---|---|---|---|---|---|---|---|
| | T Cells | | T cells | | Cells | | Cells | |
| Sample | EC50 | max | EC50 | max | EC50 | max | EC50 | max |
| ID | (nM) | MFI | (nM) | MFI | (nM) | MFI | (nM) | MFI |
| TAC2392 | 6.923 | 6492 | 26.64 | 3201 | 12.62 | 32252 | 83.83 | 6023 |
| TY25455 | 9.517 | 4853 | 10.51 | 2100 | — | — | — | — |
| TY25606 | ND | 1094 | ND | 174 | 11.66 | 51611 | 14.44 | 35964 |
| TY25715 | 28.84 | 2264 | 20.66 | 1124 | — | — | — | — |
| TY25716 | ND | 264 | ND | 159 | 14.46 | 49993 | 13.51 | 28087 |
| IgG Ctrl | ND | 129 | ND | 275 | ND | 1652 | ND | 60.4 |

### E. Binding to Human and Monkey B Cells

The abilities of the SAFEbody/bispecific antibodies to bind to human and monkey B cells, which express CD20 on surface, were tested by flow cytometry. Human and monkey PBMCs were isolated as mentioned above. Human B cells were further isolated from PBMCs using the EasySep™ Human B Cell Isolation Kit (Stem Cell Technologies). Monkey B cells were isolated from PBMCs using the Non-Human B Primate Cell Isolation Kit (Stem Cell Technologies). Purified human or monkey B cells were stained with the testing antibodies as the primary antibody and the APC-labelled anti-human-Fc antibody (BioLegend) as the secondary antibody. After washing, the cells were analyzed on the CytoFLEX flow cytometer (Beckman Coulter) and the data was analyzed by FlowJo software.

As shown in Table 34, TY25606 and TY25716 showed comparable binding affinity to human and monkey B cells, while control IgG showed no binding.

### F. Binding to CD20+ Tumor Cell Lines

The abilities of the SAFEbody/bispecific antibodies to bind to CD20+ tumor cell lines were tested by flow cytometry. Raji, SU-DHL-8, SU-DHL-4, A3KAW and NAMALWA were B-cell lymphoma cell lines with various CD20 expression levels on cell surface. Briefly, the test antibodies were serial diluted and were incubated with the tumor cells on ice. After washing, the cells were subsequently incubated with the APC-labelled anti-human-Fc secondary antibody (BioLegend) on ice. The cells were then washed with PBS prior to analysis by the CytoFLEX flow cytometer (Beckman Coulter). The data was analyzed by FlowJo software.

As shown in Table 35, all test antibodies were capable of binding to human CD20 on tumor cell surface.

TABLE 35
Binding to B-cell lymphoma cell lines

| Sample ID | | TAC2392 | TY25606 | TY25716 |
|---|---|---|---|---|
| CD3 arm | | not masked | masked | masked |
| CD20 arm | | not masked | not masked | not masked |
| Raji | Max MFI | 22046 | 24185 | 23541 |
| | EC50 (nM) | 134.8 | 35 | 28 |
| SU-DHL-8 | Max MFI | 3639 | 5794 | 5276 |
| | EC50 (nM) | 48.6 | 43 | 37.2 |
| SU-DHL-4 | Max MFI | 2290000 | 2670000 | 2480000 |
| | EC50 (nM) | 26.9 | 16 | 14.6 |
| A3KAW | Max MFI | 675000 | 942000 | 967000 |
| | EC50 (nM) | 487.3 | 120.9 | 219.6 |
| NAMALWA | Max MFI | 447000 | 656000 | 647000 |
| | EC50 (nM) | 592 | 90.4 | 161.5 |

### G. Luciferase-Based Reporter Assay

The CD20xCD3 SAFEbody/bispecific antibodies with or without MMP9 cleavage and the corresponding parental antibodies were characterized in the luciferase-based reporter assay. The Jurkat/NFAT-Luc cells (1×10⁵ cells/well), which contains a luciferase gene under the control of the NFAT response element, were mixed with CD20+ Raji cells (2×10⁴ cells/well) at the E/T ratio of 5:1 in the presence of the test antibodies with serial dilution. After incubation for 5 hours at 37° C. and 5% CO2, ONE-GLO was added and the luminescence was measured by a multiplate reader. As shown in Table 36, TY25606 and TY25716 showed significantly reduced activities when compared to their parental antibodies TY25455 and TY25715, respectively. However, the reduced activities of TY25606 and TY25715 were recovered after the masking peptides were removed by MMP9 cleavage in vitro.

TABLE 36
Summary of Jurkat/NFAT reporter assay

| Sample ID | EC50 (nM) | max RLU |
|---|---|---|
| TAC2392 | 0.144 | 7031 |
| TY25455 | 0.039 | 6750 |
| TY25606 | 13.57 | 4400 |
| TY25455 after cleavage | 0.136 | 6439 |
| TY25715 | 7.729 | 6316 |
| TY25716 | 0.086 | 4785 |
| TY25716 after cleavage | 0.214 | 6585 |
| IgG ctrl | ND | 235 |

### H. Human and Monkey T Cell-Mediated Tumor Cell Killing

The capability of CD20xCD3 bispecific antibodies to activate T cell-mediated cytolysis of tumor cells was evaluated in vitro. Briefly, human CD3+ or CD8+ T cells were purified from PBMCs using Human CD3+ T Cell Isolation Kit or Human CD8+ T Cell Isolation Kit (Stem Cell Technologies). The test antibodies were serially diluted and were incubated with Calcein-AM-labelled Raji cells (1×10⁵ cells/well) in 96-well plates. Human CD8+ or CD3+ T cells were added with the E/T ratio 10:1 or 3:1, respectively. After incubation at 37° C. for 3-6 hours, Calcein-AM release in the supernatant was measured by SpectraMax (Molecular Devices), and cytotoxicity was calculated. IFN-γ release in the supernatant was measured by ELISA (R&D Systems). CD3+/CD4+ and CD3+/CD8+ T cell activation (CD25+) was measured by flow cytometry. The data were analyzed by Graphpad Prism software.

As shown in Table 37, TY25455 and TY25715 showed stronger cytolytic activity T cell activation in CD8+ T cell-mediated killing of Raji cells at the E/T ratio of 10:1.

TABLE 37
Summary of human CD8+ and CD3+ T cell-mediated killing of Raji cells

| | CD8+T cell killing of Raji (E/T = 10:1) | | | | CD3+T cell killing of Raji (E/T = 3:1) | | | | | |
|---|---|---|---|---|---|---|---|---|---|---|
| | Cytotoxicity | | CD8+ T | | Cytotoxicity | | CD3+/CD4+ T | | CD3+/CD8+ T | |
| | to Raji Cells | | Cell Activation | | to Raji Cells | | Cell Activation | | Cell Activation | |
| | | max | | max | | max | | max | | max |
| Sample | EC50 | killing | EC50 | activation | EC50 | killing | EC50 | activation | EC50 | activation |
| ID | (nM) | (%) | (nM) | (%) | (nM) | (%) | (nM) | (%) | (nM) | (%) |
| TAC2392 | 0.047 | 33.1% | 0.057 | 74.2% | 0.019 | 17.2% | 0.317 | 26.6% | 0.104 | 17.70% |
| TY25455 | 0.010 | 31.5% | 0.017 | 80.2% | 0.003 | 15.6% | 0.077 | 33.5% | 0.048 | 22% |
| TY25606 | 15.6 | 18.0% | 4.229 | 58.3% | 15.050 | 19.1% | 6.616 | 13.4% | 4.599 | 11.80% |
| TY25715 | 0.003 | 30.3% | 0.012 | 77.6% | — | — | — | — | — | — |
| TY25716 | 57.6 | 10.3% | 14.4 | 48.9% | — | — | — | — | — | — |
| IgG Ctrl | ND | −2.7% | ND | 10.6% | ND | −2.8% | ND | 3.4% | ND | 4.25% |

### I. Human and Monkey In Vitro B Cell Killing

The capability of CD20xCD3 bispecific antibodies to activate T cells to kill endogenous B cells, which express CD20 on cell surface, was evaluated in vitro. Briefly, human and monkey PBMCs were isolated using Histopaque-1077 (Sigma) as mentioned above. Purified PBMCs (2×10⁵ cells/well) were incubated with serial-diluted test antibodies at 37° C. overnight. B cell depletion and CD8+ T cell activation was measured by flow cytometry, and the data was analyzed by FlowJo software.

As shown in Table 38, TY25455 showed stronger activities for B cell killing and CD8+ T cell activation than TY25715 and TAC2392 in the in vitro human B cell killing assay. The EC50 values of cytotoxicity were 14, 98 and 140 pM, and the EC50 values of CD8+ T cell activation were 37, 160 and 110 pM for TY25455, TY25715 and TAC2392, respectively. Meanwhile, TY25606 and TY25716 (SAFEbody/bispecific antibodies) with masking at the anti-CD3 arm showed >100-fold weaker activities and did not reach saturation signal at the highest concentration being tested.

Similar activities for the tested antibodies were also observed in the in vitro monkey B cell killing assay. The EC50 values of cytotoxicity were 41, 218 and 75 pM, and the EC50 values of CD8+ T cell activation were 87, 297 and 268 pM for TY25455, TY25715 and TAC2392, respectively. Meanwhile, TY25606 and TY25716 (SAFEbody/bispecific antibodies) with masking at the anti-CD3 arm showed >100-fold weaker activities and did not reach saturation signal at the highest concentration being tested.

TABLE 38
Summary of human and monkey endo-B killing studies

| | Human Endo-B Killing | | | | Human Endo-B Killing | | | |
|---|---|---|---|---|---|---|---|---|
| | Cytotoxicity to B cells | | CD8+ T Cell Activation | | Cytotoxicity to B cells | | CD8+ T Cell Activation | |
| | | max | | max | | max | | max |
| Sample | EC50 | killing | EC50 | activation | EC50 | killing | EC50 | activation |
| ID | (nM) | (%) | (nM) | (%) | (nM) | (%) | (nM) | (%) |
| TAC2392 | 0.14 | 83.7% | 0.11 | 52.5% | 0.075 | 76.9% | 0.268 | 64.9% |
| TY25455 | 0.014 | 92.1% | 0.037 | 54.8% | 0.041 | 81.8% | 0.087 | 73.2% |
| TY25606 | 21.8 | 92.9% | 25.8 | 20.7% | 5.323 | 69.4% | 48.91 | 35.5% |
| TY25715 | 0.098 | 92.9% | 0.16 | 52.8% | 0.218 | 88.9% | 0.297 | 67.3% |
| TY25716 | 101.4 | 86.6% | 113.2 | 10.4% | NC | 76.2% | NC | 9.8% |
| IgG ctrl | ND | 13.0% | ND | 0.8% | ND | 0.0% | ND | 0.5% |

### J. PK Studies in Tumor-Bearing Mice

Pharmacokinetics studies of the antibodies was conducted in a mouse xenograft model inoculating with Raji cells. Raji cells of 5×10⁶ in DPBS were subcutaneously implanted into M-NSG mice (Shanghai Model Organisms). Three mice per dosing group were intravenously injected with the test antibodies when tumor volumes reached 60-150 mm³. Serum concentrations of TY25455 and TY25606 were determined by ELISA, in which the anti-human IgG (Fc specific) antibody was used for capture and the HRP-labeled anti-human IgG (Fab specific) antibody for detection.

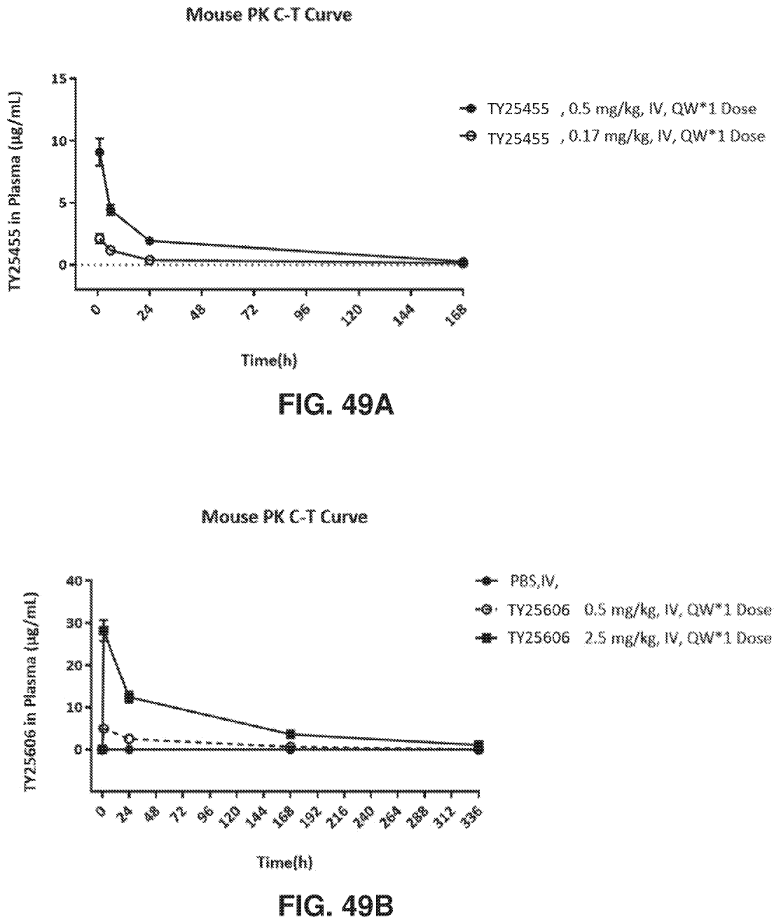

As shown in FIGS. 49A-49B, TY25455 and TY25606 showed comparable pharmacokinetics in mice.

K. Monkey Toxicity/PD Studies with Single Dosing

A pilot non-GLP toxicity and pharmacology study was performed in cynomolgus monkeys (Macaca fascicularis) to determine the ability of the CD20xCD3 SAFEbody/bispecific antibodies to deplete B cell population in animals. Male animals received a single intravenous infusion of TAC2392 (0.3 mg/kg), TY25455 (0.3 mg/kg), or TY25606 (0.3, 3 and 30 mg/kg). Blood was drawn immediately prior to dosing to establish baseline levels for B and T cells in these animals. Drugs of different doses were administered by intravenous infusion and blood was drawn at different time points post dosing for the analysis of B and T cell levels by flow cytometry. Serum samples were also analyzed for cytokine levels (IFN-γ and IL-2) using ELISA method. Serum concentrations ofTY25455 and TY25606 were measured by ELISA.

As shown in FIGS. 50A-50D, administration of TAC2392, TY25455, TY25606, TY25715 and TY25716 resulted in depletion of circulating B cells to baseline levels by the first time point measured (3 hours), followed by gradual recovery around 10 days after dosing. T cell levels were also monitored in the experiment. A transient loss of circulating T cells was observed after dosing. T cell levels returned to baseline levels by the day 7 time point and maintained or were above the levels until the end of the experiment. Serum cytokine levels for the antibodies exhibited a dose- and time-dependent response that is consistent with T cell activation. The cytokine levels elicited by TY25606 at 30 mg/kg were comparable to those elicited by TAC2392 or TY25455 at 0.3 mg/kg. The cytokine levels elicited by TY25715 at 30 mg/kg were lower than those elicited by TAC2392 or TY25715 at 0.3 mg/kg. These results indicated that the SAFEbody/bispecific antibody format effectively reduced cytokine release in vivo. As shown in Table 39, TY25606 and TY25716 showed improved half-life, comparing to TAC2392 and the parental antibodies TY25455 and TY25715.

TABLE 39
Pharmacokinetics analysis of the cynomolgus monkey studies

| | TAC2392 | TY25455 | TY25606 | TY25606 | TY25606 | TY25715 | TY25716 | TY25716 | TY25716 |
|---|---|---|---|---|---|---|---|---|---|
| Dosage | (0.3 mg/kg) | (0.3 mg/kg) | (0.3 mg/kg) | (3 mg/kg) | (30 mg/kg) | (0.3 mg/kg) | (0.3 mg/kg) | (3 mg/kg) | (30 mg/kg) |
| Animal # | 1 | 1 | 1 | 1 | 1 | 1 | 1 | 1 | 1 |
| Tmax | 0.33 | 0.33 | 0.33 | 0.33 | 0.33 | 0.33 | 0.33 | 1.00 | 0.33 |
| Cmax | 1.74 | 0.41 | 3.99 | 44.41 | 125.24 | 1.15 | 8.55 | 17.04 | 72.64 |
| λz | 0.21 | 0.33 | 0.10 | 0.07 | 0.06 | 0.11 | 0.15 | 0.16 | 0.07 |
| C_last | 0.22 | 0.06 | 0.57 | 10.98 | 48.82 | 0.20 | 0.77 | 5.18 | 56.92 |
| t½z | 3.32 | 2.07 | 7.02 | 9.56 | 12.32 | 6.10 | 4.74 | 4.37 | 10.53 |
| (days) | | | | | | | | | |
| AUClast | 1.94 | 0.59 | 18.84 | 121.99 | 1097.25 | 2.42 | 36.37 | 63.07 | 180.41 |
| AUCinf | 3.02 | 0.78 | 24.62 | 273.38 | 1964.91 | 4.14 | 41.64 | 95.70 | 1045.56 |
| R_AUC | 35.74 | 24.18 | 23.47 | 55.38 | 44.16 | 41.55 | 12.65 | 34.10 | 82.75 |
| AUMClast | 2.53 | 0.85 | 93.16 | 335.27 | 6569.38 | 6.25 | 161.22 | 191.41 | 275.06 |
| AUMCinf | 12.02 | 2.16 | 232.61 | 3482.41 | 34135.81 | 33.40 | 270.99 | 625.48 | 16019.61 |
| CL | 99348.88 | 386076.22 | 12183.07 | 10973.59 | 15267.87 | 72541.89 | 7204.43 | 31347.29 | 28692.63 |
| MRTlast | 0.80 | 0.94 | 4.44 | 2.25 | 5.49 | 2.09 | 3.93 | 2.53 | 1.02 |
| MRTinf | 3.48 | 2.28 | 8.95 | 12.24 | 16.87 | 7.58 | 6.01 | 6.04 | 14.82 |
| Vss | 345921.94 | 882048.15 | 108992.96 | 134296.67 | 257610.20 | 549664.86 | 43282.49 | 189203.43 | 425267.55 |
| Vz | 476536.15 | ###### | 123372.03 | 151304.91 | 271326.12 | 638590.21 | 49257.52 | 197573.93 | 436082.94 |

### L. In Vivo Efficacy Studies Using a Mouse Xenograft Model

The in vivo anti-tumor efficacy of the antibodies was tested in a mouse xenograft model inoculating with human PBMC and Raji cells. Raji cells of 5×10⁶ in DPBS were subcutaneously implanted into M-NSG mice (Shanghai Model Organisms). Human PBMCs of 2.5×10⁷ in DPBS were intravenously injected three days later. The mice were started to be intravenously treated with test antibodies or control IgG (N=6 each group) once per week when tumor volumes reached ˜100 mm³. Tumor volumes and body weights were measured twice per week until the study was finished. The mice would be euthanized if tumor volume exceeded 3000 mm³ or weight loss exceeded 20%.

As shown in FIG. 48, the parental bispecific antibodies TY25455 and TY25715, and the SAFEbody/bispecific antibody TY25606 were effective in preventing tumor growth in vivo in the presence of PBMC effector cells. There was no difference in the body weight of the mice among different experimental groups (data not shown).

### Example 12. Comparison of Activatable CD20xCD3 Antibody with Plamotamab

Targeting of CD20-expressing tumor cells with anti-CD20 monoclonal antibodies (mAb) has proven to be highly successful for the treatment of B-cell malignancies. The addition of the anti-CD20 mAb Rituximab to chemotherapy has dramatically improved the outcome for these patients; nevertheless, disease relapse or recurrence still occurs. Recent clinical data illustrate the effectiveness of bispecific molecules, often referred to as T cell engagers (TCEs), that redirect the patient's endogenous T cells to eliminate tumor cells. Such therapeutics concomitantly bind to CD3 on T cells and a target antigen on target cancer cells. CD20xCD3 TCEs, such as Odronextamab and Plamotamab, have demonstrated promising clinical activities in B-cell malignancies. However, these therapies also suffer from incidences (6%) of Grade ≥3 cytokine release syndrome (CRS), even after step-up dosing strategies are implemented.

Activatable CD20xCD3 bispecific antibody TY25606 has a masked medium affinity anti-CD3 scFv arm, an unmasked anti-CD20 Fab arm, and an Fc of human IgG1 kappa having an N297A mutation in the CH2 domain and TYM13 mutations in the CH3 domain. The masking moiety is linked to the anti-CD3 scFv domain via a linker having an MMP-9 cleavage site. The parental CD20xCD3 bispecific antibody TY25455 has an unmasked anti-CD3 scFv arm.

In vitro studies showed that the TY25606 has higher potencies than the benchmark Plamotamab analog (Plamotamab) for the binding to human B cells and CD20-positive Raji tumor cells. On the other hand, TY25606 has significantly reduced binding to the human CD3δ/ε protein dimer (>80-fold masking efficiency compared with Plamotamab and the unmasked parental molecule) and no binding to human CD3+, CD4+, and CD8+ T cells. See, Tables 50A-50B.

TABLE 50A
Binding to CD3δ/ε protein dimer.

| Antibody | Plamotamab | TY25455 | TY25606 |
|---|---|---|---|
| EC50 (nM) | 2.1 | 24.7 | 2098 |

TABLE 50B
Binding to human T cells, B cells and Raji cells.

| | CD3+ | CD4+ | CD8+ | B | Raji |
|---|---|---|---|---|---|
| | T, | T, | T, | cells, | cells, |
| | EC50 | EC50 | EC50 | EC50 | EC50 |
| Antibody | (nM) | (nM) | (nM) | (nM) | (nM) |
| Plamotamab | 11.78 | 13.11 | 10.12 | 200.3 | 134.8 |
| TY25455 | 21.69 | 18.08 | 25.37 | — | — |
| TY25606 | >2000 | >2000 | >2000 | 78.21 | 35 |
| Isotype Ctrl | >2000 | >2000 | >2000 | >2000 | >8000 |

Consistent with these results, TY25606 shows decreased ability (>140-fold) compared with Plamotamab and the unmasked parental molecule to activate CD8+ T cells (Table 50C) and to induce T cell-mediated killing (Table 50D) in the presence of Raji tumor cells in vitro.

TABLE 50C
Jurkat-NFAT Reporter Assay in Presence of Raji cells.

| | | | | TY25606 | Isotype |
|---|---|---|---|---|---|
| Antibody | Plamotamab | TY25455 | TY25606 | cleaved | Control |
| EC50 (nM) | 0.144 | 0.039 | 13.57 | 0.086 | >200 |

TABLE 50D
CD8+ T cell-mediated killing of Raji cells.

| Antibody | Plamotamab | TY25455 | TY25606 |
|---|---|---|---|
| EC50 (nM) | 0.113 | 0.008 | 16.61 |

Similar to Plamotamab, TY25455 had similar potency for the induction of killing of endogenous B cells and activation of endogenous CD8+ T cells within human PBMCs in vitro. TY25606 showed minimal activity in these assays. See, Tables 50E-50F.

TABLE 50E
Killing of B cells within human PBMCs.

| Antibody | Plamotamab | TY25455 | TY25606 | Isotype Ctrl |
|---|---|---|---|---|
| EC50 (nM) | 1.445 | 0.3698 | >160 | >160 |

TABLE 50F
Activation of CD8+ T cells within human PBMCs.

| Antibody | Plamotamab | TY25455 | TY25606 | Isotype Ctrl |
|---|---|---|---|---|
| EC50 (nM) | 0.798 | 0.2841 | >160 | >160 |

In contrast to its compromised activities in vitro, TY25606 demonstrated strong anti-tumor effect in vivo. In the human PBMC-engrafted mouse model containing Raji xenograft tumors, dosing with 1.5 mg/kg TY25606 and 0.17 mg/kg Plamotamab resulted in about 85% tumor growth inhibition. In exploratory toxicology studies in cynomolgus monkeys, TY25606 was as effective as Plamotamab (at 0.3 mg/kg) at inducing B cell depletion from peripheral blood. The cytokine release of TY25606 was minimal throughout the treatment course for doses up to 3 mg/kg. Importantly, even 30 mg/kg of TY25606 resulted in >1 to 2-fold less maximum cytokine release (e.g., IFN-g and IL-2) in monkey blood compared with 0.3 mg/kg Plamotamab, implicating ˜100-fold safety margin for TY25606 in terms of cytokine induction. Taken together, these results indicate that TY25606 is a differentiated CD20xCD3 TCE with strong anti-tumor activities with significantly reduced liabilities of cytokine induction at the projected therapeutic dose levels.

### Example 13. Generation and Characterization of HER2xCD3 Bispecific Antibodies

### A. Generation of HER2xCD3 Bispecific Antibodies

Ty27151 was Generated from Ty25238 by the Addition of a Masking Moiety and cleavable moiety (MM-CM) region on both the anti-CD3 and anti-HER2 arms. TY27008 was generated from TY25238 by the addition of a masking moiety and cleavage moiety (MM-CM) region on the anti-CD3 arm only (see schematic in FIG. 5B). The amino acid sequences of the MM-CM region for the anti-CD3 and anti-HER2 arms of TY27151 and TY27008 are provided in Table 40, below.

TABLE 40
Amino acid sequences of TY27151 and TY27008 masking moieties and cleavage
moieties

| | | MM | | CM | |
|---|---|---|---|---|---|
| | | | SEQ | Amino Acid | SEQ ID |
| IgG ID | Arm | Amino Acid Sequence | ID NO | Sequence | NO |
| TY27151 | anti-CD3 | EVGSYPYDDPDCPSHESD | 417 | GGGPLGLAGSGGS | 77 |
| | arm | CDQ | | | |
| | anti- | ESDACDADPFDCQA | 419 | GGGPLGLAGSGGS | 77 |
| | HER2 | | | | |
| | arm | | | | |
| TY27008 | anti-CD3 | EVGSYPYDDPDCPSHESD | 417 | GGGPLGLAGSGGS | 77 |
| | arm | CDQ | | | |

The amino acid sequences of the full-length heavy and light chains ofTY27151 and TY27008 are provided in Table 41, below. Heavy chain 1 (HC1) and light chain (LC1) are the anti-HER2 portions of the TY27151, and heavy chain 2 (HC2) is the anti-CD3 portion of TY27151. Heavy chain 1 (HC1) and light chain (LC1) are the anti-HER2 portions of the TY27008, and heavy chain 2 (HC2) is the anti-CD3 portion of TY27008. The heavy chains sequences below are provided with the C-terminal lysine residue included. Masking moiety sequences are bolded and underlined.

TABLE 41
Amino acid sequences of TY27151 and TY27008 heavy and light chains

| | Region of | | SEQ ID |
|---|---|---|---|
| IgG ID | antibody | Amino Acid Sequence | NO |
| TY27151 | LC1 | ESDACDADPFDCQAGGGPLGLAGSGGSDIQMTQSPSSLSASVGD | 83 |
| | | RVTITCRASQDVNTAVAWYQQKPGKAPKLLIYSASFLYSGVPSR | |
| | | FSGSRSGTDFTLTISSLQPEDFATYYCQQHYTTPPTFGQGTKVE | |
| | | IKRTVAAPSVFIFPPSDEQLKSGTASVVCLLNNFYPREAKVQWK | |
| | | VDNALQSGNSQESVTEQDSKDSTYSLSSTLTLSKADYEKHKVYA | |
| | | CEVTHQGLSSPVTKSFNRGEC | |
| TY27151 | HC1 | EVQLVESGGGLVQPGGSLRLSCAASGFNIKDTYIHWVRQAPGKG | 84 |
| | | LEWVARIYPTNGYTRYADSVKGRFTISADTSKNTAYLQMNSLRA | |
| | | EDTAVYYCSRWGGDGFYAMDYWGQGTLVTVSSASTKGPSVFPLA | |
| | | PSSKSTSGGTAALGCLVKDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPELLGGPSVFLFPPKPKDTLMISRTPEVTC | |
| | | VVVDVSHEDPEVKFNWYVDGVEVHNAKTKPREEQYASTYRVVSV | |
| | | LTVLHQDWLNGKEYKCKVSNKALPAPIEKTISKAKGQPREPQVY | |
| | | TLPPSRKKLTKNQVKLTCLVKGFYPSDIAVEWESNGQPENNYKT | |
| | | TPPVLDCDGSFFLYSKLTVDKSRWQQGNVFSCSVMHEALHNHYT | |
| | | QKSLSLSPGK | |
| TY27151 | HC2 | EVGSYPYDDPDCPSHESDCDQGGGPLGLAGSGGSQAVVTQEPSL | 85 |
| | | TVSPGGTVTLTCGSSTGAVTTSNYPNWVQQKPGQAPRGLIGGTN | |
| | | KRAPGVPARFSGSLLGGKAALTLSGAQPEDEAEYYCALWYSNLW | |
| | | VFGGGTKLTVLGGGGSGGGGSGGGGSGGGGSEVQLVESGGGLVQ | |
| | | PGGSLRLSCAASGFTFNTYAINWVRQAPGKGLEWVGRIRSKYNN | |
| | | YATYYAESVKGRFTISRDDSKNTLYLQINSLRAEDTAVYYCVRH | |
| | | GNFGTSYVSWFAYWGQGTLVTVSSEPKSSDKTHTCPPCPAPELL | |
| | | GGPSVFLFPPKPKDTLMISRTPEVTCVVVDVSHEDPEVKFNWYV | |
| | | DGVEVHNAKTKPREEQYASTYRVVSVLTVLHQDWLNGKEYKCKV | |
| | | SNKALPAPIEKTISKAKGQPREPQVYTDPPSRDELTKNQVSLTC | |
| | | LVDGFYPSDIAVEWESNGQPENCYKTTPPVLDSDGSFFLYSKLT | |
| | | VDKSRWQQGNVFSCSVMHEALHNHYTQDSLSLSPGK | |
| TY27008 | LC1 | DIQMTQSPSSLSASVGDRVTITCRASQDVNTAVAWYQQKPGKAP | 683 |
| | | KLLIYSASFLYSGVPSRFSGSRSGTDFTLTISSLQPEDFATYYC | |
| | | QQHYTTPPTFGQGTKVEIKRTVAAPSVFIFPPSDEQLKSGTASV | |
| | | VCLLNNFYPREAKVQWKVDNALQSGNSQESVTEQDSKDSTYSLS | |
| | | STLTLSKADYEKHKVYACEVTHQGLSSPVTKSENRGEC | |
| TY27008 | HC1 | EVQLVESGGGLVQPGGSLRLSCAASGFNIKDTYIHWVRQAPGKG | 684 |
| | | LEWVARIYPTNGYTRYADSVKGRFTISADTSKNTAYLQMNSLRA | |
| | | EDTAVYYCSRWGGDGFYAMDYWGQGTLVTVSSASTKGPSVFPLA | |
| | | PSSKSTSGGTAALGCLVKDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPELLGGPSVFLFPPKPKDTLMISRTPEVTC | |
| | | VVVDVSHEDPEVKFNWYVDGVEVHNAKTKPREEQYASTYRVVSV | |
| | | LTVLHQDWLNGKEYKCKVSNKALPAPIEKTISKAKGQPREPQVY | |
| | | TLPPSRKKLTKNQVKLTCLVKGFYPSDIAVEWESNGQPENNYKT | |
| | | TPPVLDCDGSFFLYSKLTVDKSRWQQGNVFSCSVMHEALHNHYT | |
| | | QKSLSLSPGK | |
| TY27008 | HC2 | EVGSYPYDDPDCPSHESDCDQGGGPLGLAGSGGSQAVVTQEPSL | 685 |
| | | TVSPGGTVTLTCGSSTGAVTTSNYPNWVQQKPGQAPRGLIGGTN | |
| | | KRAPGVPARFSGSLLGGKAALTLSGAQPEDEAEYYCALWYSNLW | |
| | | VFGGGTKLTVLGGGGSGGGGSGGGGSGGGGSEVQLVESGGGLVQ | |
| | | PGGSLRLSCAASGFTFNTYAINWVRQAPGKGLEWVGRIRSKYNN | |
| | | YATYYAESVKGRFTISRDDSKNTLYLQINSLRAEDTAVYYCVRH | |
| | | GNFGTSYVSWFAYWGQGTLVTVSSEPKSSDKTHTCPPCPAPELL | |
| | | GGPSVFLFPPKPKDTLMISRTPEVTCVVVDVSHEDPEVKFNWYV | |
| | | DGVEVHNAKTKPREEQYASTYRVVSVLTVLHQDWLNGKEYKCKV | |
| | | SNKALPAPIEKTISKAKGQPREPQVYTDPPSRDELTKNQVSLTC | |
| | | LVDGFYPSDIAVEWESNGQPENCYKTTPPVLDSDGSFFLYSKLT | |
| | | VDKSRWQQGNVFSCSVMHEALHNHYTQDSLSLSPGK | |

To generate the bispecific antibodies, plasmids encoding the heavy chain, light chain, and scFv-Fc chain were transiently transfected into mammalian cells. Bispecific antibody-containing cell culture supernatants were harvested 7 days after transfection by centrifugation at 14000 g for 30 minutes, and were filtered through a sterile filter (0.22 μm). Antibodies were purified by protein A affinity chromatography using MabSelect SuRe prepacked columns (GE Healthcare) and were subsequently buffer exchanged in 20 mM histidine (pH 5.5) buffer. The purity of the proteins are shown in Table 42 below.

TABLE 42
The new bispecific antibodies and SEC-HPLC purity

| | | | SEC-HPLC purity | | |
|---|---|---|---|---|---|
| | | Fc | HMW | Monomer | LMW |
| IgG ID | Format | mutant | (%) | (%) | (%) |
| TY25238 | Bispecific | TYM13 | 2.3 | 93.9 | 3.8 |
| | antibody | | | | |
| TY27008 | HER2 × | TYM13 | 11.9 | 88.1 | 0 |
| | SAFE-CD3 | | | | |
| TY27151 | SAFE-HER2 × | TYM13 | 9.6 | 90.4 | 0 |
| | SAFE-CD3 | | | | |

### B. Binding to CD3 and HER2

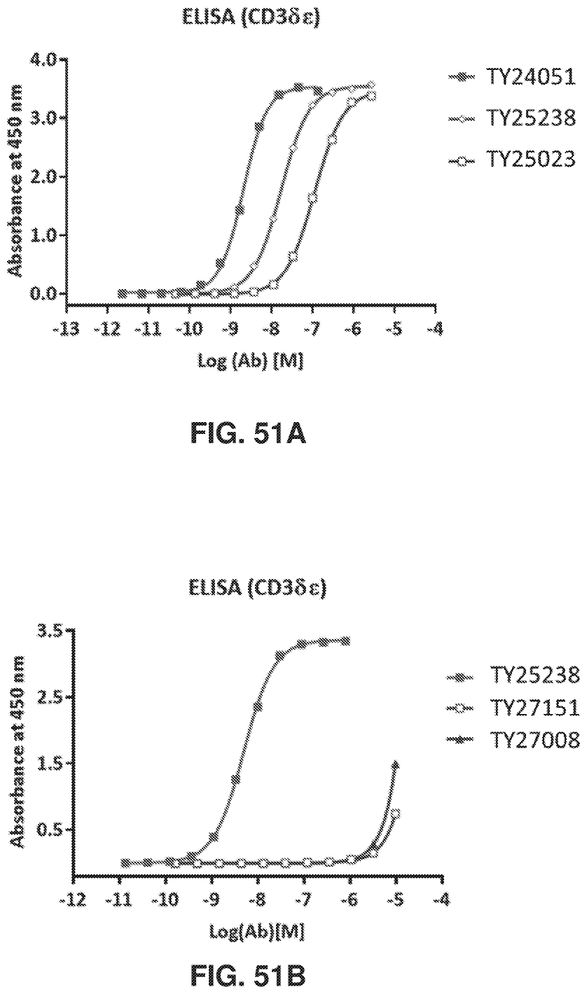

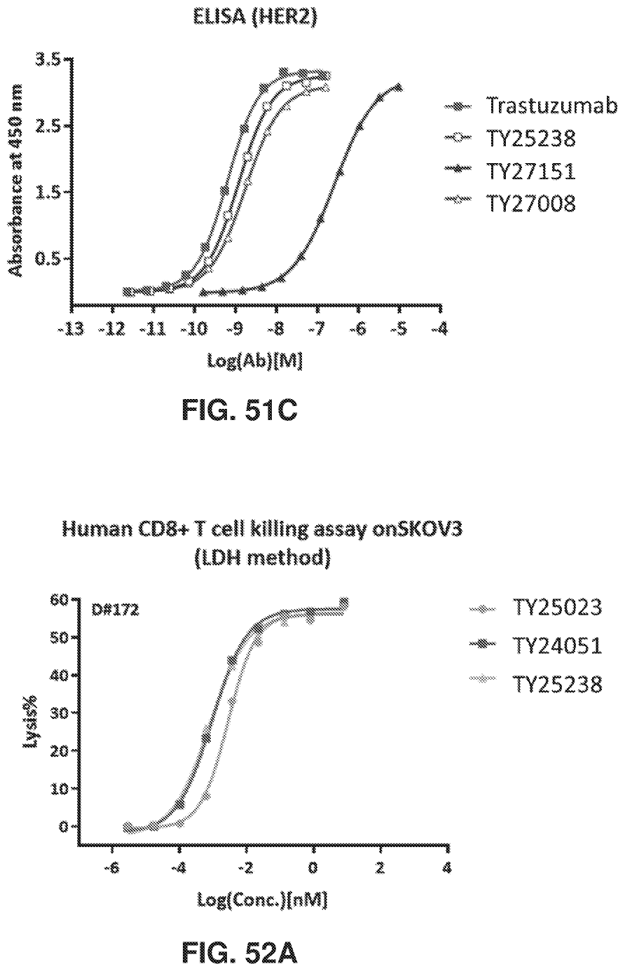

The binding affinities of the HER2xCD3 bispecific antibodies were analyzed through enzyme-linked immunosorbent assays (ELISAs) (see FIGS. 51A-51C). 2 μg/mL of human CD3 (ε and δ chain heterodimer) or human HER2 fused with human Fc fragment, were prepared and used to coat the ELISA plate at 2-8° C. overnight. After washing and blocking, 50 μL serial diluted IgGs were added and incubated at 37° C. for 1 hour. Plates were washed three times and then incubated with 50 μL/well TMB substrate at room temperature for about 20 minutes. Absorbance at 450 nm was measured after the reaction was stopped. The concentration of each antibody that produced half-maximal binding to the antigen is reported as the EC₅₀ in nM.

As shown in Table 43 and FIGS. 51A-51C, The activatable bispecific antibodies TY27008 and TY27151, show much lower affinity to CD3 than their parental antibody TY25238. The activatable bispecific antibody TY27151 also show low affinity to HER2 than TY25238.

TABLE 43
ELISA EC50s of bispecific antibodies

| | ELISA (CD3δε) | ELISA (CD3δε) | ELISA (HER2) |
|---|---|---|---|
| Sample ID | EC50 (nM) | EC50 (nM) | EC50 (nM) |
| TY24051 | 2.03 | — | — |
| TY25023 | 110.6 | — | — |
| TY25238 | 16.9 | 4.96 | 1.18 |
| TY27008 | — | ~8.07E+06 | 1.74 |
| TY27151 | — | ~2.47E+06 | 259.1 |
| Trastuzumab | — | | 0.64 |

To compare functional activity between TY24051 and TY25023, the antibodies were expressed, purified and evaluated for antigen-dependent bispecific antibody-mediated tumor cell killing activity. For in vitro cytotoxicity assays, human CD8+ T-cells were isolated from fresh human blood and mixed with HER2-positive tumor cells with different antigen expression levels along with increasing amounts of bispecific antibody overnight (target cells:1×10⁴ cells/well, E:T=10:1).

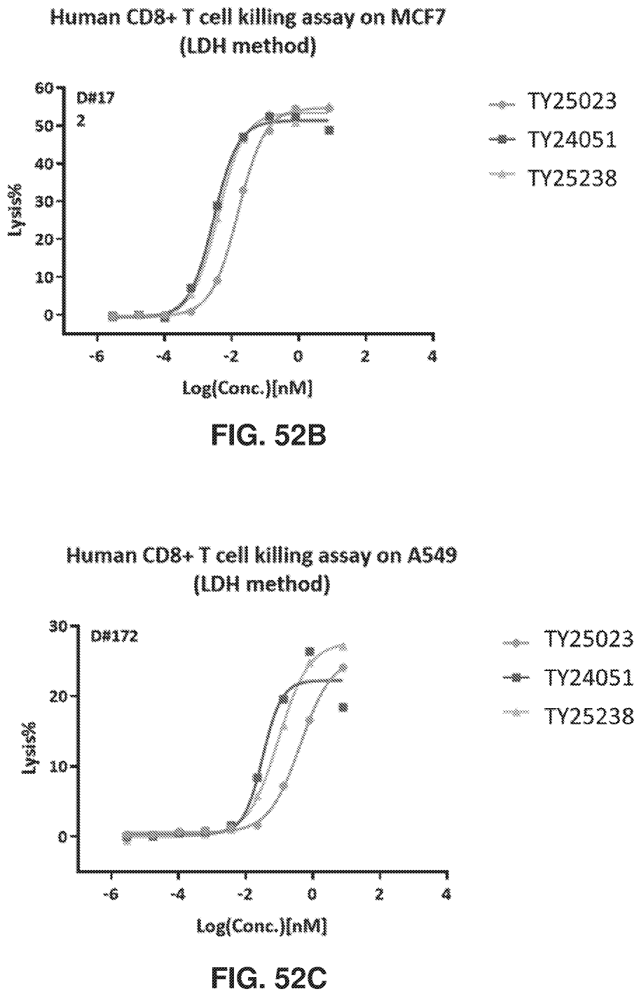

As shown in FIGS. 52A-52C, for different cell lines, compared with TY24051 and TY25023, the tumor killing activity of the new bispecific antibody TY25238 show comparable activity as TY24051, and is apparently more potent than TY25023. The EC50 values of the cell killing activities are listed in Table 44.

TABLE 44
Cytotoxicity EC50 values

EC50(pM) LDH Kit

| Sample ID | SKOV3 (High) | MCF-7 (Medium) | A549 (Low) |
|---|---|---|---|
| TY24051 | 0.931 | 2.90 | 31.9 |
| TY25023 | 2.82 | 15.6 | 422.5 |
| TY25238 | 0.790 | 3.90 | 94.4 |

### C. Cleavage of Masking Moieties

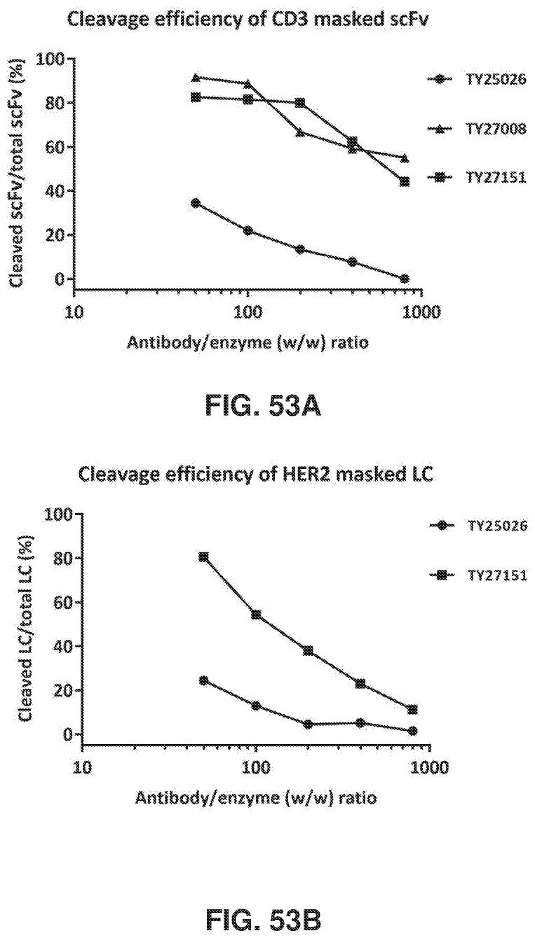

The CM sequences in TY27151 and TY27008 were designed to improve their cleavage efficiency by MMPs. As shown in FIGS. 53A-53B, both CMs of the anti-HER2 and anti-CD3 arms of TY27151 and TY27008 were easier to be cleaved by MMP-9 than TY25026.

### D. Repeated SK-OV-3 Binding

Dose-dependent binding activity of the parental, activatable, or cleaved HER2xCD3 bispecific antibodies on SK-OV-3 tumor cells, was measured using flow cytometry. Briefly, SKOV-3 cells were seeded in 96-well plates at 1.0×10⁵ per well and incubated with serially diluted test antibodies for 30 minutes at 4° C. in 1% FBS/1640 buffer. After that, the cells were washed twice with DPBS and further incubated with secondary APC-anti-human IgG Fc antibody for 30 minutes at 4° C. Finally, the cells were washed twice with DPBS and suspended in FACS buffer for flow cytometry analysis. Then MFI values vs. concentrations were analyzed with Flowjo and Data were further fitted with four-parameter non-linear regression to get EC50 values by GraphPad Prism software.

As shown in Table 45, TY27008, parental and cleaved forms showed very similar binding (similar EC50) to HER2 molecules on SKOV-3 cells. TY27151 showed about 265-fold masking efficiency, compared with its parental antibody TY25238.

TABLE 45
EC₅₀ of SK-OV-3 full dose binding

| Abs | EC50(nM) | Span | AUC_{(-2.4951~2.699)} |
|---|---|---|---|
| TAC2416 | 9.67 | 55593 | 103421 |
| TY25238 | 4.08 | 56424 | 123633 |
| TY27008-Cleaved | 6.10 | 56465 | 112416 |
| TY27151-Cleaved | 7.42 | 54985 | 106335 |
| TY27008 | 5.86 | 58984 | 118695 |
| TY27151 | 1082 | 31735 | 36746 |

### E. In Vitro Pharmacology Studies

The in vitro tumor killing activity of parental, activatable, or cleaved HER2xCD3 bispecific antibodies on SK-OV-3, MCF-7, JIMT-1, or HT55 tumor cell lines was measured using an LDH assay. Briefly, the human PBMCs were purified with Ficoll gradient solution—Histopaque 1077 (Sigma) from one healthy donor blood. SK-OV-3, MCF-7, JIMT-1, or HT55 (1×10⁴ cells/well) cells were prepared to incubate with serially diluted test articles for 30 min at 37° C., and the human PBMC cells (10×10⁴cells/well) were added and incubated for 20 h (E:T=10:1). The in vitro tumor killing activity was analyzed with CytoTox96 non-Radioactive cytotoxicity assay kit (Promega G1780).

As shown in Table 46, when SK-OV-3, MCF-7, JIMT-1, or HT55 was used as target cells, TAC2416 (reference antibody described in US20150133640A1, with polypeptide chains of SEQ ID NOs: 43-45), parental TY25238 and cleaved HER2xCD3 bispecific antibodies showed very similar in vitro killing activities (i.e., similar EC50). The CD3-masked TY27008 exhibited relatively modest in vitro killing activity. TY27151 with both HER2 and CD3 arms masked showed the weakest in vitro killing activity among all the tested antibodies. In addition, our study indicated that killing activity (both EC50 and Emax) is generally correlated with target expression level on the cell lines.

TABLE 46
Killing activity on SK-OV-3, MCF-7, JIMT-1, or HT55 tumor cell lines

| | SK-OV-3 EC50(nM) | | | MCF-7 EC50(nM) | | |
|---|---|---|---|---|---|---|
| Sample | | Activation- | Activation- | | Activation- | Activation- |
| ID | Cytotoxicity | CD4 | CD8 | Cytotoxicity | CD4 | CD8 |
| TAC2416 | 0.0036 | 0.0025 | 0.0015 | 0.0105 | 0.0407 | 0.0557 |
| TY25238 | 0.0012 | 0.0018 | 0.0013 | 0.0104 | 0.0504 | 0.0561 |
| TY27008- | 0.0016 | 0.0028 | 0.0021 | 0.0094 | 0.0699 | 0.1195 |
| Cleaved | | | | | | |
| TY27151- | 0.0013 | 0.0029 | 0.0017 | 0.0083 | 0.1422 | 0.1690 |
| Cleaved | | | | | | |
| TY27008 | 0.0451 | 0.0470 | 0.0588 | 0.3051 | 0.4987 | 0.7666 |
| TY27151 | 1.4730 | 2.6970 | 1.1810 | NA | NA | NA |
| HER2 | 58678.0000 | | | 1161.0000 | | |
| Expression | | | | | | |
| (MFI) | | | | | | |
| | JIMT-1 EC50(nM) | | | HT55 EC50(nM) | | |
| Sample | | Activation- | Activation- | | Activation- | Activation- |
| ID | Cytotoxicity | CD4 | CD8 | Cytotoxicity | CD4 | CD8 |
| TAC2416 | 0.0181 | 0.3480 | 0.4799 | 0.0440 | 0.2147 | 0.2763 |
| TY25238 | 0.0154 | 0.1406 | 0.1581 | 0.0465 | 0.3787 | 0.7057 |
| TY27008- | 0.0191 | 0.2163 | 0.3680 | 0.0705 | 0.7066 | NA |
| Cleaved | | | | | | |
| TY27151- | 0.0166 | 0.2857 | 0.3566 | 0.0455 | 1.3830 | NA |
| Cleaved | | | | | | |
| TY27008 | 0.4495 | 0.8766 | 1.3070 | 0.6744 | 2.5040 | NA |
| TY27151 | NA | NA | NA | NA | NA | NA |
| HER2 | 8009.0000 | | | 2447.0000 | | |
| Expression | | | | | | |
| (MFI) | | | | | | |

### F. HER2 Expressing Tumors (SK-OV3) Model

Immunodeficient M-NSG mice (n=6 per group, female, 8-9 weeks old) were transplanted with 5×10⁶ PBMC through i.v. injection. After 3 days 2×10⁶ SK-OV3 cells were inoculated subcutaneously. Treatment began at Day 11 post tumor inoculation when the average tumor volume reached about 130 mm³. The mice were administered with Vehicle, anti-HER2xanti-CD3 bispecific antibody TY25023 or anti-HER2x anti-CD3 bispecific antibody TY25238 at 1 mg/kg, 0.2 mg/kg or 0.04 mg/kg by i.p. injection. The mice were administered these Abs twice per week for a total of four doses. Tumor growth was monitored twice a week and reported as the mean tumor volume±s.e.m. over time.

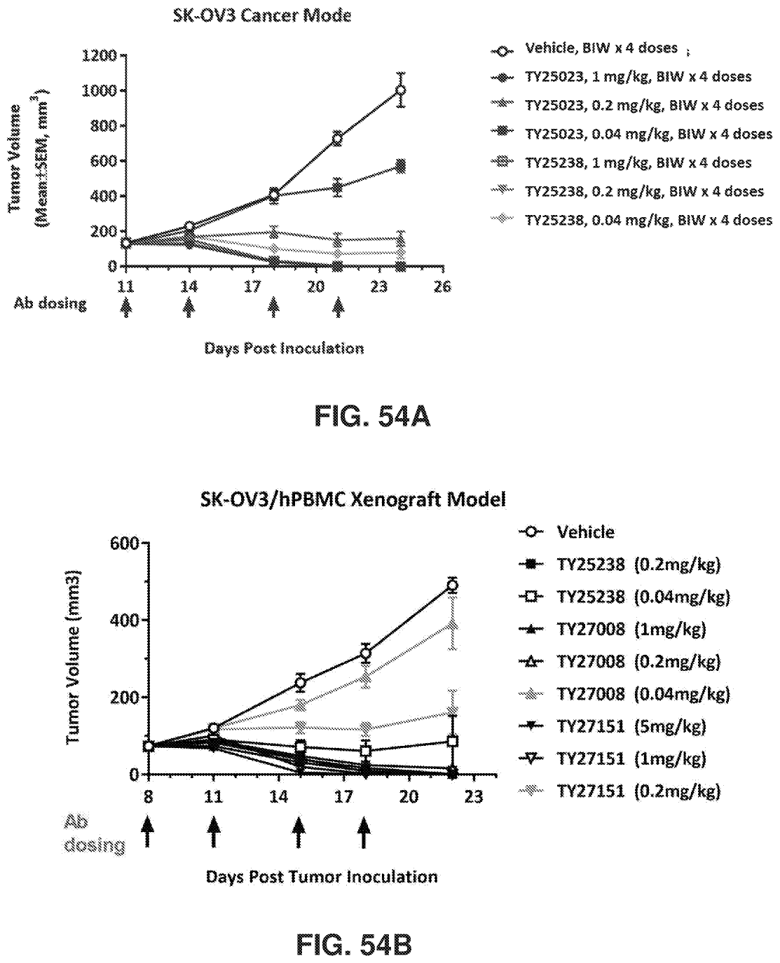

As shown in FIG. 54A, the bispecific antibody TY25238 showed significantly stronger anti-tumor efficacy than TY25023 at both 0.2 mg/kg and 0.04 mg/kg.

In a second experiment, immunodeficient M-NSG mice (n=6 per group, female, 8-9 weeks old) were transplanted with 5×10⁶ PBMC through i.p. injection. Seven days later, the mice were inoculated subcutaneously with 2×10⁶ SK-OV3 cells. Treatment began at Day 8 post tumor inoculation when the average tumor volume reached about 75 mm³. The mice were administered with Vehicle, anti-HER2xanti-CD3 bispecific parental antibody TY25238 at 0.2 and 0.04 mg/kg, or anti-HER2xanti-CD3 bispecific single masked bispecific antibody TY27008 at 1 mg/kg, 0.2 mg/kg and 0.04 mg/kg, or two arms-masked anti-HER2xanti-CD3 bispecific antibody TY27151 at 5 mg/kg, 1 mg/kg and 0.2 mg/kg, or anti-HER2xanti-CD3 bispecific benchmark antibody TAC2416 at 0.04 mg/kg by i.p. injection. The mice were administered these Abs twice per week for a total of six doses. Tumor growth was monitored twice a week and reported as the mean tumor volume±s.e.m. over time.

As shown in FIG. 54B, the parental antibody TY25238 showed much stronger anti-tumor efficacy than both TY27008 and TY27151. While the single masked bispecific antibody TY27008 also showed much stronger anti-tumor efficacy than the two arms-masked bispecific antibody TY27151.

### G. PK Profile in Cynomolgus Monkey

A pilot non-GLP toxicology and pharmacology study was performed in healthy male cynomolgus monkeys (Macaca fascicularis) to determine the pharmacodynamics and toxicological profile of the CD3xHER2 bispecific antibodies in these animals. Each animal received either a repeat parental bispecific antibody or masked bispecific antibody, as summarized in Table 47.

TABLE 47
Study design.

| | Number/Sex | Dosing | Dosing | Dosage |
|---|---|---|---|---|
| Sample ID | of Animals | schedule | route | (mg/kg) |
| TY25238 | 1 male | QW × 2 | i.v. | 0.02 |
| | 1 male | QW × 1 | i.v. | 0.2 |
| TY27008 | 1 male | QW × 2 | i.v. | 2 |
| | 1 male | QW × 2 | i.v. | 10 |
| TY27151 | 1 male | QW × 2 | i.v. | 10 |
| | 1 male | QW × 2 | i.v. | 30 |
| | 1 male | QW × 1 | i.v. | 60 |

TY25238, TY27008 and TY27151 were administrated by i.v. bolus injection and PK samples were withdrawn at pre-dose (0 h), 0.25 h, 1 h, 8 h, 24 h, 48 h, 72 h, 96 h and 168 h post dosing. Cytokine samples were withdrawn at pre-dose (0 h), 3 h, 8 h, 24 h, 48 h, 72 h, 168 h, 171 h, 192 h, 240 h, and 336 h post dosing. Serum was extracted at each time point and cytokine concentrations were analyzed by ELISA. Plasma was extracted at each time point and blood concentrations were analyzed by ELISA to determine the PK parameters. For total drug concentration testing, Goat anti-human IgG antibody Monkey ads (SouthernBiotech, Cat #2049-01) was used to capture the drug from the plasma, followed by adding HRP-Goat anti-human IgG (Fab specific) (Sigma, Cat #A0293) as secondary antibody. For active drug forms, antigen (CD3 or HER2) proteins were used to capture the active drugs from the plasma, followed by adding HRP-Goat anti-human IgG (Fab specific) (Sigma, Cat #A0293) as secondary antibody.

All treatments were well tolerated in monkeys except TY25238 at 0.2 mg/kg (animal death following the first dose and remarkable cytokine release) and TY27008 at 10 mg/kg (remarkable body weight loss and clinical signs).

Clinical signs were mainly observed in most of the treatments, including decreased activity and loose feces in TY25238 at 0.2 mg/kg, occasional loose feces in TY27151 at 10 mg/kg and TY27008 at 2 mg/kg, and loose feces, vomit, lethargy, loss of appetite in TY27008 at 10 mg/kg.

Hematological changes were similar across treatment: depletion of lymphocytes and increase in neutrophils; decreased in red cell mass with compensatory increase in reticulocytes which were still present at the last measured timepoint. Changes in serum chemistry were also similar across treatments: transient increases in ALT, AST, bilirubin, and CK levels.

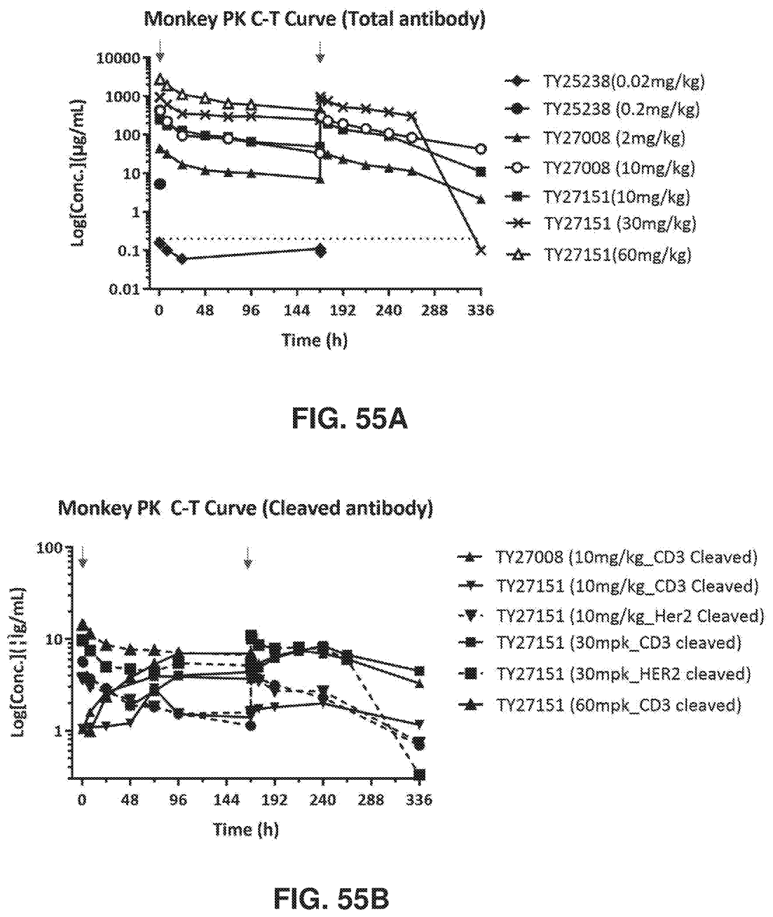

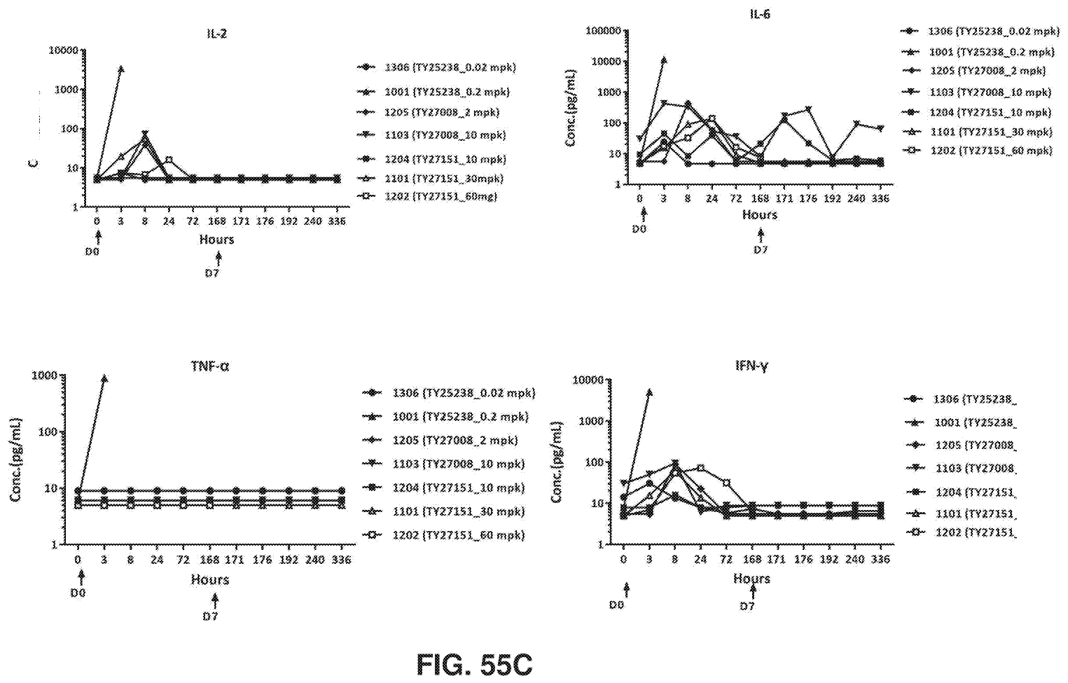

As shown in FIG. 55A, only Cmax was acquired as the animal was found dead after the 1st dosing of parental TY25238. TY27008 exhibit higher drug exposure and faster clearance than TY27151 which was manifested by higher Cmax and short T1/2 time (or higher CL), respectively after the 1st dosing. After receiving the second repeated dose, similar exposure was observed for TY27008 and TY27151, but the clearance of TY27151 was faster than TY27008 which may be caused by production of treatment induced anti-drug antibody (ADA) in TY27151 treated animal.

As shown in FIG. 55B, only active form can be detected in plasma of TY27008 treated animal, and the active drug concentrations accumulated following time after both the 1st and 2nd dosing. No active forms can be detected in plasma of TY27151 treated animal.

As shown in FIG. 55C, for the monkey administered with 0.2 mg/kg TY25238, all the cytokines (IL-6, IFN-r, IL-2 and TNF-α) were remarkably elevated at 3 h after TCE treatment and with the monkey died around 6 h post-dosing, most likely due to the acute cytokine release syndrome. Weak cytokine releases (<100 ng/mL) were detected in the monkey treated with 0.02 mg/mL TY25238 and the monkey was well tolerated at this dose level. For the double-masked activatable antibody TY27151, no cytokine release was significantly detectable at low dose or just minimal cytokine release was detected at higher doses (30 or 60 mg/mL). For the single-masking activatable antibody, lower cytokine release could be detectable within 3-8 hours after first and second doses at 2 and 10 mg/kg. The monkey dosed with 10 mg/kg TY27008 showed serious signs of toxicity after 2^{nd} dose. TY27151 showed remarkably reduced cytokine release than the parental antibody. ˜100-fold lower IL-6 peak levels were observed in TY27151 administered animals at doses up to 60 mg/kg than parental antibody at 0.2 mg/kg. Other cytokines including IL-2, TNF-α, also demonstrated much reduced levels. Monkey received a single dose at 0.2 mg/kg of TY27151 parental TCE died 6 h post-dose, whereas monkeys can tolerate well to the masked TY27151 up to 60 mg/kg in this study.

A summary of PK data is provided in Table 48 below.

TABLE 48
PK data in Cynomolgus Monkeys.

| Dosage | Subject | Cmax | C_last | t½z | AUClast | AUCinf | CL | Vss |
|---|---|---|---|---|---|---|---|---|
| 10 mg/kg | TY27008 | 439.45 | 34.63 | 92.43 | 14771.14 | 19389.06 | 515.75 | 57391.53 |
| (Day 1) | TY27151 | 263.07 | 47.63 | 121.41 | 14977.30 | 23319.94 | 428.82 | 69677.26 |
| 10 mg/kg | TY27008 | 310.08 | 42.75 | 71.81 | 19473.86 | 23902.59 | 418.36 | 40415.42 |
| (Day 7) | TY27151 | 309.83 | 12.27 | 38.69 | 15013.14 | 15697.84 | 637.03 | 34164.91 |

### Example 14. CD3 Signaling Activation by Anti-HER2xCD3 Activatable/Bispecific Antibodies

HER2xCD3 activatable antibody/bispecific antibodies with or without MMP9 cleavage and the corresponding parental antibodies were characterized in a luciferase-based CD3 reporter assay examining activation of CD3 signaling.

Jurkat/NFAT-Luc cells (1×10⁵ cells/well), which contained a luciferase gene under the control of the NFAT response element, were mixed with SK-OV-3 cells (2×10⁴ cells/well) at the E/T ratio of 5:1 in the presence of the test antibodies with serial dilutions. After incubation for about 5 hours at 37° C. and 5% CO2, ONE-GLO was added and the luminescence was measured by a multiplate reader.

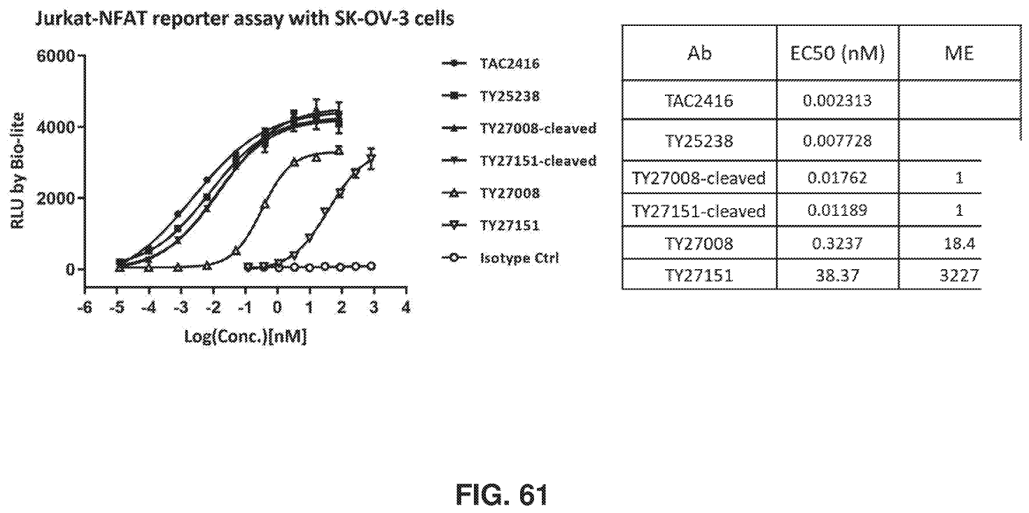

As shown in FIG. 61, reference TAC2416, and activatable antibody's cleaved forms (TY27008-cleaved and TY27151-cleaved) showed very similar CD3 reporter gene activities, compared to their parental antibody TY25238. However, the activatable antibody TY27008 and TY27151 showed significant reduced CD3 activities.

### Example 15. Anti-Tumor Efficacy of Anti-HER2xCD3 Bispecific Antibodies

Anti-tumor efficacy of HER2xCD3 activatable antibody/bispecific antibodies was tested in an in vivo preclinical tumor model.

Immunodeficient M-NSG mice (n=7 per group, female, 8-9 weeks old) were transplanted with 5×10⁶ PBMC through i.p. injection. Seven days later, the mice were inoculated subcutaneously with 5×10⁶ HT55 cells, which are low HER2-expressing tumor cells. Treatment began at Day 8 post tumor inoculation when the average tumor volume reached about 130 mm³. The mice were administered with Vehicle, anti-HER2xanti-CD3 bispecific parental antibody TY25238 at 0.2 and 1 mg/kg, or anti-HER2xanti-CD3 bispecific single masked activatable antibody TY27008 at 0.2, 1 and 5 mg/kg, or anti-HER2xanti-CD3 bispecific double masked activatable antibody TY27151 at 0.4, 2 and 10 mg/kg, by i.p. injection. The mice were administered these Abs twice per week for a total of six doses. Tumor growth was monitored twice a week and reported as the mean tumor volume±s.e.m. over time.

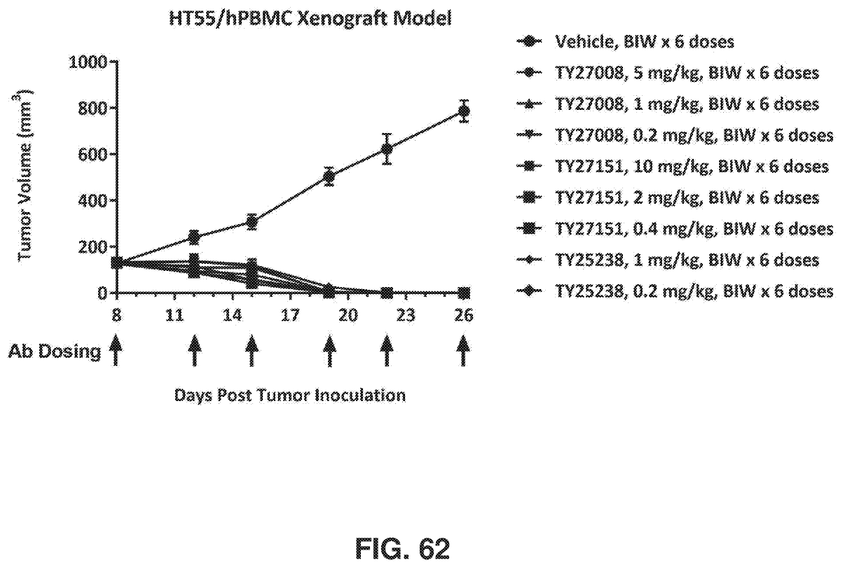

As shown in FIG. 62, all the tested antibodies showed strong anti-tumor effect at all dose levels.

Anti-tumor efficacy of HER2xCD3 activatable antibody/bispecific antibodies was compared with other anti-HER2 antibodies in an in vivo preclinical tumor model.

Immunodeficient M-NSG mice (n=7 per group, female, 8-9 weeks old) were transplanted with 5×10⁶ PBMC through i.p. injection. Seven days later, the mice were inoculated subcutaneously with 5×10⁶ HT55 cells. Treatment began at Day 8 post tumor inoculation when the average tumor volume reached about 83 mm³. The mice were administered with Vehicle, anti-HER2xanti-CD3 bispecific double masked activatable antibody TY27151 at 0.025, 0.1 and 0.4 mg/kg, Trastuzumab or DS-8201 ADC at 0.4 mg/kg, by i.p. injection. The mice were administered these Abs twice per week for a total of six doses. Tumor growth was monitored twice a week and reported as the mean tumor volume±s.e.m. over time.

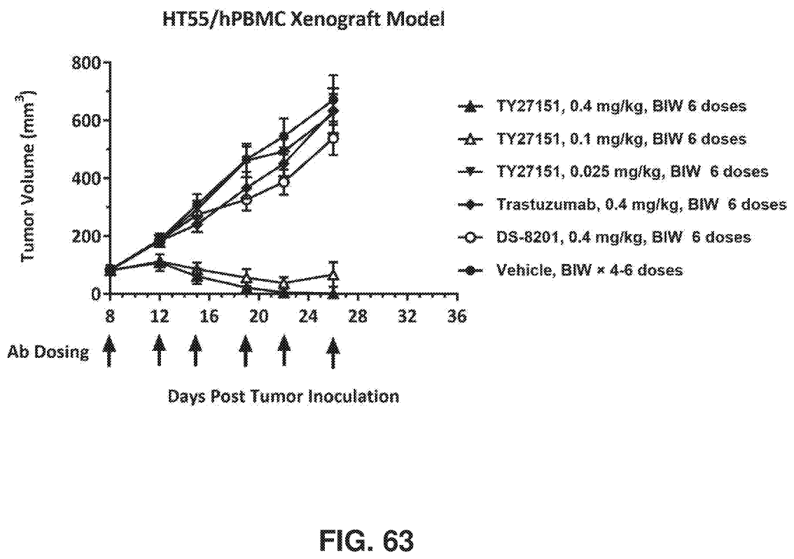

As shown in FIG. 63, only TY27151 at 0.1 and 0.4 mg/kg doses showed strong anti-tumor effect. TY27151 at lower dose level of 0.025 mg/kg, Trastuzumab or DS-8201 at 0.4 mg/kg dose level, did not have significant antitumor efficacy.

Anti-tumor efficacy of HER2xCD3 activatable antibody/bispecific antibodies was tested in combination with anti-CD137 mAb in an in vivo preclinical tumor model.

C57BL/6-hCD3E mice (n=8 per group, female, 8-9 weeks old) were inoculated subcutaneously with 1×10⁶ MC38-hHER2 cells. Treatment began at Day 5 post tumor inoculation when the average tumor volume reached about 105 mm³. The mice were administered with Vehicle, anti-HER2xanti-CD3 bispecific double masked activatable antibody TY27151 at 5 mg/kg, anti-CD137 antibody AG10131-mouse IgG2a mAb at 5 mg/kg, or combination of TY27151 and anti-CD137 AG10131-mouse IgG2a by i.p. injection. The mice were administered these Abs twice per week for a total of four doses. Tumor growth was monitored twice a week and reported as the mean tumor volume±s.e.m. over time. 61 days post initial tumor inoculation, mice with complete tumor regression in the combination treatment group (6/8) were rechallenged with MC38-HER2 tumor cells. Naïve mice were also inoculated with MC38-HER2 tumor cells at the same time as control.

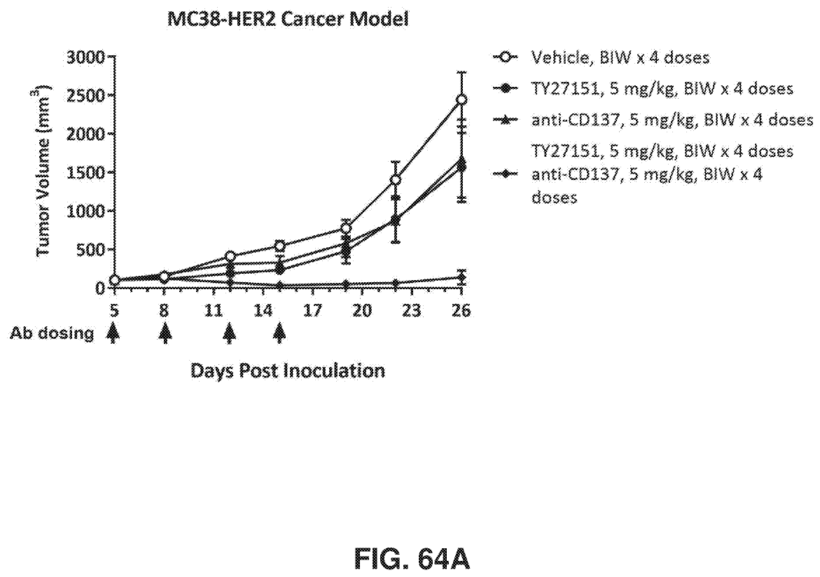

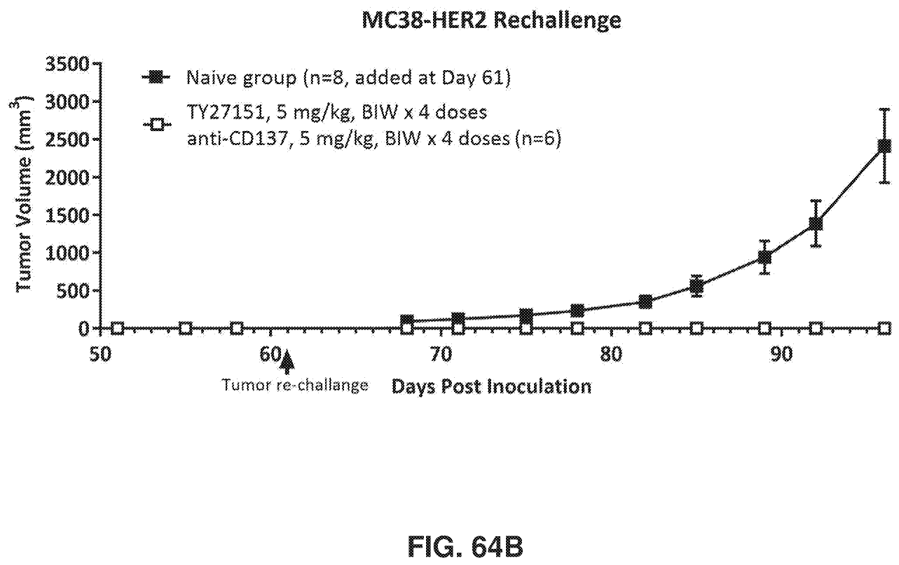

As shown in FIG. 64A, the anti-HER2xanti-CD3 bispecific double masked activatable antibody TY27151 showed strong synergistic anti-tumor effect with the anti-CD137 mAb in this model. FIG. 64B indicated that the combination treatment between the anti-HER2xanti-CD3 bispecific double masked activatable antibody TY27151 and the anti-CD137 antibody synergized to induce long-lasting anti-tumor memory responses even when the treatment was stopped.

Anti-tumor efficacy of HER2xCD3 activatable antibody/bispecific antibodies was also tested in combination with anti-PD-1 mAb in an in vivo preclinical tumor model. The anti-PD-1 antibody is a control anti-human PD-1 IgG4 antibody with cross-reactivity to mouse, monkey, and human PD-1 comprising a heavy chain variable domain comprising the amino acid sequence of

(SEQ ID NO: 708)
QVQLVQSGAEVKKPGSSVKVSCKASGFTFTTYYISWVRQAPGQGLEYLG
YINMGSGGTNYNEKFKGRVTITADKSTSTAYMELSSLRSEDTAVYYCAI
IGYFDYWGQGTMVTVSS
and
a light chainvariable domain comprising the amino
acid sequence of
(SEQ ID NO: 709)
DVVMTQSPLSLPVTLGQPASISCRSSQSLLDSDGGTYLYWFQQRPGQSP
RRLIYLVSTLGSGVPDRFSGSGSGTDFTLKISRVEAEDVGVYYCMQLTH
WPYTFGQGTKLEIKR.

Immunodeficient M-NSG mice (n=7 per group, female, 8-9 weeks old) were transplanted with 5×10⁶ PBMC through i.p. injection. Seven days later, the mice were inoculated subcutaneously with 2×10⁶ SK-OV3 cells. Treatment began at Day 8 post tumor inoculation when the average tumor volume reached about 70 mm³. The mice were administered with hIgG4 isotype control at 5 mg/kg, anti-HER2xanti-CD3 double masked activated bispecific antibody TY27151 at 0.2 mg/kg in combination with isotype ctrl at 5 mg/kg, anti-PD-1 mAb 2E5 at 5 mg/kg, or TY27151 at 0.2 mg/kg in combination with 2E5 at 5 mg/kg by i.p. injection. The mice were administered these Abs twice per week for a total of five doses. Tumor growth was monitored twice a week and reported as the mean tumor volume±s.e.m. over time.

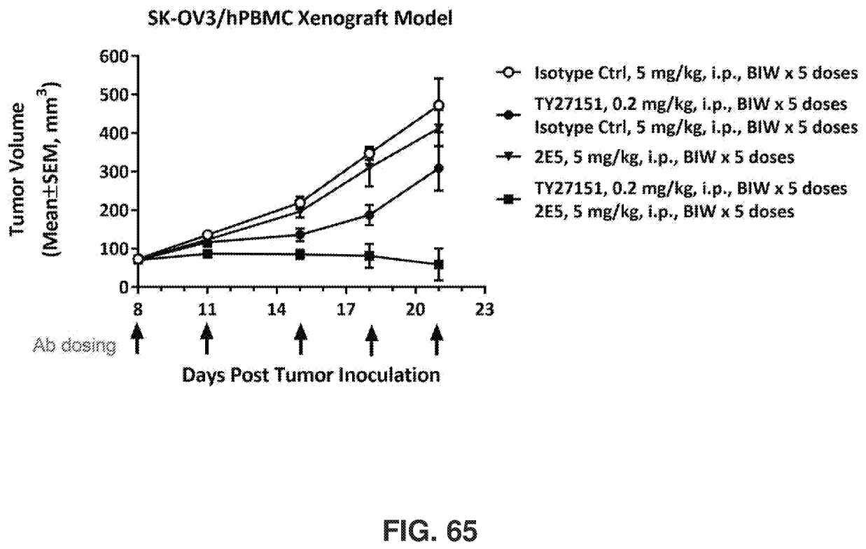

As shown in FIG. 65, the anti-HER2xanti-CD3 double masked activated bispecific antibody TY27151 showed strong synergistic anti-tumor effects with the anti-PD-1 mAb 2E5 in this model.

What is claimed is:

1. A multispecific antibody comprising:
a) a first antigen-binding fragment that specifically binds CD3, wherein the first antigen-binding fragment is fused to a first masking moiety (MM1) via a first cleavable moiety (CM1) comprising a first cleavage site, wherein the first antigen-binding fragment comprises a first immunoglobulin light chain variable domain (VL1) and a first immunoglobulin heavy chain variable domain (VH1) of an anti-CD3 antibody, wherein the MM1 is fused to the N-terminus of the VL1 via the CM1, wherein the MM1 inhibits binding of the multispecific antibody to CD3 when the CM1 is not cleaved, and wherein the MM1 comprises an amino acid sequence selected from the group consisting of SEQ ID NOs: 35, 417, and 597-599 and the CM1 comprises an amino acid sequence selected from the group consisting of SEQ ID NOs: 77, 127-129, 418, 420, 431 and 477-490, and 516-555; and
b) a second antigen-binding fragment that specifically binds a target antigen;
wherein the MM1 competes with CD3 to specifically bind the CD3-binding moiety; and wherein
i. the VH1 comprises
a CDR-H1 comprising the amino acid sequence of SEQ ID NO: 376,
a CDR-H2 comprising the amino acid sequence of SEQ ID NO: 391, and
a CDR-H3 comprising the amino acid sequence of SEQ ID NO: 378; and
the VL1 comprises
a CDR-L1 comprising the amino acid sequence of SEQ ID NO: 396,
a CDR-L2 comprising the amino acid sequence of SEQ ID NO: 380, and
a CDR-L3 comprising the amino acid sequence of SEQ ID NO: 381;
ii. the VH1 comprises
a CDR-H1 comprising the amino acid sequence of SEQ ID NO: 390,
a CDR-H2 comprising the amino acid sequence of SEQ ID NO: 392, and
a CDR-H3 comprising the amino acid sequence of SEQ ID NO: 395; and
the VL1 comprises
a CDR-L1 comprising the amino acid sequence of SEQ ID NO: 397,
a CDR-L2 comprising the amino acid sequence of SEQ ID NO: 380, and
a CDR-L3 comprising the amino acid sequence of SEQ ID NO: 400;
iii. the VH1 comprises
a CDR-H1 comprising the amino acid sequence of SEQ ID NO: 390,
a CDR-H2 comprising the amino acid sequence of SEQ ID NO: 392, and
a CDR-H3 comprising the amino acid sequence of SEQ ID NO: 395; and
the VL1 comprises
a CDR-L1 comprising the amino acid sequence of SEQ ID NO: 396,
a CDR-L2 comprising the amino acid sequence of SEQ ID NO: 380, and
a CDR-L3 comprising the amino acid sequence of SEQ ID NO: 401;
iv. the VH1 comprises
a CDR-H1 comprising the amino acid sequence of SEQ ID NO: 390,
a CDR-H2 comprising the amino acid sequence of SEQ ID NO: 393, and
a CDR-H3 comprising the amino acid sequence of SEQ ID NO: 395; and
the VL1 comprises
a CDR-L1 comprising the amino acid sequence of SEQ ID NO: 397,
a CDR-L2 comprising the amino acid sequence of SEQ ID NO: 380, and
a CDR-L3 comprising the amino acid sequence of SEQ ID NO: 381;
v. the VH1 comprises
a CDR-H1 comprising the amino acid sequence of SEQ ID NO: 376,
a CDR-H2 comprising the amino acid sequence of SEQ ID NO: 393, and
a CDR-H3 comprising the amino acid sequence of SEQ ID NO: 395; and
the VL1 comprises
a CDR-L1 comprising the amino acid sequence of SEQ ID NO: 396,
a CDR-L2 comprising the amino acid sequence of SEQ ID NO: 380, and
a CDR-L3 comprising the amino acid sequence of SEQ ID NO: 401;
vi. the VH1 comprises
a CDR-H1 comprising the amino acid sequence of SEQ ID NO: 376,
a CDR-H2 comprising the amino acid sequence of SEQ ID NO: 393, and
a CDR-H3 comprising the amino acid sequence of SEQ ID NO: 395; and
the VL1 comprises
a CDR-L1 comprising the amino acid sequence of SEQ ID NO: 397,
a CDR-L2 comprising the amino acid sequence of SEQ ID NO: 380, and
a CDR-L3 comprising the amino acid sequence of SEQ ID NO: 400;
vii. the VH1 comprises
a CDR-H1 comprising the amino acid sequence of SEQ ID NO: 376,
a CDR-H2 comprising the amino acid sequence of SEQ ID NO: 393, and
a CDR-H3 comprising the amino acid sequence of SEQ ID NO: 395; and
the VL1 comprises
a CDR-L1 comprising the amino acid sequence of SEQ ID NO: 398,
a CDR-L2 comprising the amino acid sequence of SEQ ID NO: 399, and
a CDR-L3 comprising the amino acid sequence of SEQ ID NO: 400;
viii. the VH1 comprises
a CDR-H1 comprising the amino acid sequence of SEQ ID NO: 390,
a CDR-H2 comprising the amino acid sequence of SEQ ID NO: 394, and
a CDR-H3 comprising the amino acid sequence of SEQ ID NO: 395; and
the VL1 comprises
a CDR-L1 comprising the amino acid sequence of SEQ ID NO: 397,
a CDR-L2 comprising the amino acid sequence of SEQ ID NO: 380, and
a CDR-L3 comprising the amino acid sequence of SEQ ID NO: 381;
ix. the VH1 comprises
a CDR-H1 comprising the amino acid sequence of SEQ ID NO: 390,
a CDR-H2 comprising the amino acid sequence of SEQ ID NO: 391, and
a CDR-H3 comprising the amino acid sequence of SEQ ID NO: 395; and
the VL1 comprises
a CDR-L1 comprising the amino acid sequence of SEQ ID NO: 396,
a CDR-L2 comprising the amino acid sequence of SEQ ID NO: 380, and
a CDR-L3 comprising the amino acid sequence of SEQ ID NO: 381;
x. the VH1 comprises
a CDR-H1 comprising the amino acid sequence of SEQ ID NO: 390,
a CDR-H2 comprising the amino acid sequence of SEQ ID NO: 394, and
a CDR-H3 comprising the amino acid sequence of SEQ ID NO: 395; and
the VL1 comprises
a CDR-L1 comprising the amino acid sequence of SEQ ID NO: 396,
a CDR-L2 comprising the amino acid sequence of SEQ ID NO: 380, and
a CDR-L3 comprising the amino acid sequence of SEQ ID NO: 381;
xi. the VH1 comprises
a CDR-H1 comprising the amino acid sequence of SEQ ID NO: 376,
a CDR-H2 comprising the amino acid sequence of SEQ ID NO: 391, and
a CDR-H3 comprising the amino acid sequence of SEQ ID NO: 378; and
the VL1 comprises
a CDR-L1 comprising the amino acid sequence of SEQ ID NO: 397,
a CDR-L2 comprising the amino acid sequence of SEQ ID NO: 380, and
a CDR-L3 comprising the amino acid sequence of SEQ ID NO: 400;
xii. the VH1 comprises
a CDR-H1 comprising the amino acid sequence of SEQ ID NO: 390,
a CDR-H2 comprising the amino acid sequence of SEQ ID NO: 393, and
a CDR-H3 comprising the amino acid sequence of SEQ ID NO: 378; and
the VL1 comprises
a CDR-L1 comprising the amino acid sequence of SEQ ID NO: 396,
a CDR-L2 comprising the amino acid sequence of SEQ ID NO: 380, and
a CDR-L3 comprising the amino acid sequence of SEQ ID NO: 381;
xiii. the VH1 comprises
a CDR-H1 comprising the amino acid sequence of SEQ ID NO: 390,
a CDR-H2 comprising the amino acid sequence of SEQ ID NO: 391, and
a CDR-H3 comprising the amino acid sequence of SEQ ID NO: 378; and
the VL1 comprises
a CDR-L1 comprising the amino acid sequence of SEQ ID NO: 396,
a CDR-L2 comprising the amino acid sequence of SEQ ID NO: 380, and
a CDR-L3 comprising the amino acid sequence of SEQ ID NO: 381; or
xiv. the VH1 comprises
a CDR-H1 comprising the amino acid sequence of SEQ ID NO: 390,
a CDR-H2 comprising the amino acid sequence of SEQ ID NO: 391, and
a CDR-H3 comprising the amino acid sequence of SEQ ID NO: 378; and
the VL1 comprises
a CDR-L1 comprising the amino acid sequence of SEQ ID NO: 397,
a CDR-L2 comprising the amino acid sequence of SEQ ID NO: 380, and
a CDR-L3 comprising the amino acid sequence of SEQ ID NO: 381.

2-7. (canceled)

8. The multispecific antibody of claim 1, wherein the first antigen-binding fragment is a scFv comprising, from N-terminus to C-terminus, VL1, a linker, and VH1.

9. The multispecific antibody of claim 8, wherein the multispecific antibody comprises a first polypeptide, a second polypeptide, and a third polypeptide, wherein:
(i) the first polypeptide comprises a structure represented by the formula:
$$VH 2 - CH 1 - hinge - CH 2 - first CH 3 ; \qquad ( 1 a )$$
(ii) the second polypeptide comprises a structure represented by the formula:
$$MM 1 - CM 1 - VL 1 - VH 1 - hinge - CH 2 - second CH 3 ; \qquad ( 1 b )$$
and
(iii) the third polypeptide comprises a structure represented by the formula:
$$VL 2 - CL ; \qquad ( 1 c )$$
wherein:
CL is an immunoglobulin light chain constant domain;
CH1 is an immunoglobulin heavy chain constant domain 1;
CH2 is an immunoglobulin heavy chain constant domain 2;
first CH3 is a first immunoglobulin heavy chain constant domain 3;
second CH3 is a second immunoglobulin heavy chain constant domain 3;
hinge is an immunoglobulin hinge region connecting the CH1 and CH2 domains;
wherein the VL1 and the VH1 associate to form a scFv that specifically binds CD3; and
wherein the VL2 and the VH2 associate to form a Fv that specifically binds the target antigen.

10-13. (canceled)

14. The multispecific antibody of claim 1, wherein the second antigen-binding fragment comprises a second immunoglobulin light chain variable domain (VL2) and a second immunoglobulin heavy chain variable domain (VH2) of an antibody that specifically binds the target antigen.

15. The multispecific antibody of claim 14, wherein the second antigen-binding fragment is selected from the group consisting of a Fab, a Fv, a scFab and a scFv.

16. The multispecific antibody of claim 15, wherein the second antigen-binding fragment is fused to a second masking moiety (MM2) via a second cleavable moiety (CM2), wherein the CM2 comprises a second cleavage site, wherein the MM2 inhibits binding of the multispecific antibody to the target antigen when the CM2 is not cleaved, and wherein the multispecific antibody binds the target antigen via the second antigen-binding fragment when the CM2 is cleaved.

17. The multispecific antibody of claim 16, wherein the MM2 is fused to the N-terminus of the VL2 via the CM2.

18. The multispecific antibody of claim 17, wherein the multispecific antibody comprises a first polypeptide, a second polypeptide, and a third polypeptide, wherein:
(i) the first polypeptide comprises a structure represented by the formula:
$$VH 2 - CH 1 - hinge - CH 2 - first CH 3 ; \qquad ( 2 a )$$
(ii) the second polypeptide comprises a structure represented by the formula:
$$MM 1 - VL 1 - VH 1 - hinge - CH 2 - second CH 3 ; \qquad ( 2 b )$$
and
(iii) the third polypeptide comprises a structure represented by the formula:
$$MM 2 - CM 2 - VL 2 - CL ; \qquad ( 2 c )$$
wherein:
CL is an immunoglobulin light chain constant domain;
CH1 is an immunoglobulin heavy chain constant domain 1;
CH2 is an immunoglobulin heavy chain constant domain 2;
first CH3 is a first immunoglobulin heavy chain constant domain 3;
second CH3 is a second immunoglobulin heavy chain constant domain 3;
hinge is an immunoglobulin hinge region connecting the CH1 and CH2 domains;
wherein the VL1 and the VH1 associate to form a scFv that specifically binds CD3; and
wherein the VL2 and the VH2 associate to form a Fv that specifically binds the target antigen.

19. The multispecific antibody of claim 1, wherein the CD3 is human CD3.

20-24. (canceled)

25. The multispecific antibody of claim 1, wherein the VH1 comprises
a CDR-H1 comprising the amino acid sequence of SEQ ID NO: 390,
a CDR-H2 comprising the amino acid sequence of SEQ ID NO: 392, and
a CDR-H3 comprising the amino acid sequence of SEQ ID NO: 395; and
the VL1 comprises
a CDR-L1 comprising the amino acid sequence of SEQ ID NO: 397,
a CDR-L2 comprising the amino acid sequence of SEQ ID NO: 380, and
a CDR-L3 comprising the amino acid sequence of SEQ ID NO: 400.

26. The multispecific antibody of claim 1, wherein the VH1 comprises
a CDR-H1 comprising the amino acid sequence of SEQ ID NO: 390,
a CDR-H2 comprising the amino acid sequence of SEQ ID NO: 394, and
a CDR-H3 comprising the amino acid sequence of SEQ ID NO: 395; and
the VL1 comprises
a CDR-L1 comprising the amino acid sequence of SEQ ID NO: 397,
a CDR-L2 comprising the amino acid sequence of SEQ ID NO: 380, and
a CDR-L3 comprising the amino acid sequence of SEQ ID NO: 381.

27. The multispecific antibody of claim 1, wherein the first antigen-binding fragment comprises a VH1 and a VL1, and wherein:
a) the VH1 comprises the amino acid sequence according to Formula (VII): EVQLVESGGGLVX₁PGGSLRLSCAASGFTFX₂X₃YAIX₄WVRQAPGKGLEWVX₅RIRS KYNNYATYYAX₆SVKX₇RFTISRDX₈SKNTLYLQX₉NSLRAEDTAVYYCX₁₀RHGNX₁₁ GX₁₂SYVSWFAYWGQGTLVTVSS (SEQ ID NO: 388), wherein X₁ is K or Q, X₂ is N or S, X₃ is S or T, X₄ is H or N, X₅ is G or S, X₆ is D or E, X₇ is D or G, X₈ is D or N, X₉ is I or L, X₁₀ is A or V, X₁₁ is F or Y, X₁₂ is N or T; and
b) the VL1 comprises the amino acid sequence according to Formula (VIII): X₁AVVTQEPSLTVSPGGTVTLTCX₂SSTGAVTTSNYX₃NWX₄QQKPGQAPRGLIGGTX₅X₆RAPGX₇PARFSGSLLGGKAALTLSGAQPEDEAEYYCALWYSX₈X₉WVFGGGTKL TVL (SEQ ID NO: 389), wherein X₁ is E or Q, X₂ is A, G, P, or R, X₃ is A or P, X₄ is F or V, X₅ is K or N, X₆ is F or K, X₇ is A, I, T, or V, X₈ is A, D, N, or T, and X₉ is H or L.

28. (canceled)

29. (canceled)

30. The multispecific antibody of claim 27, wherein:
a) the VH1 comprises the amino acid sequence of SEQ ID NO: 402, and the VL1 comprises the amino acid sequence of SEQ ID NO: 403;
b) the VH1 comprises the amino acid sequence of SEQ ID NO: 402, and the VL1 comprises the amino acid sequence of SEQ ID NO: 404;
c) the VH1 comprises the amino acid sequence of SEQ ID NO: 405, and the VL1 comprises the amino acid sequence of SEQ ID NO: 406;
d) the VH1 comprises the amino acid sequence of SEQ ID NO: 407, and the VL1 comprises the amino acid sequence of SEQ ID NO: 404;
e) the VH1 comprises the amino acid sequence of SEQ ID NO: 407, and the VL1 comprises the amino acid sequence of SEQ ID NO: 403;
f) the VH1 comprises the amino acid sequence of SEQ ID NO: 407, and the VL1 comprises the amino acid sequence of SEQ ID NO: 408;
g) the VH1 comprises the amino acid sequence of SEQ ID NO: 409, and the VL1 comprises the amino acid sequence of SEQ ID NO: 408;
h) the VH1 comprises the amino acid sequence of SEQ ID NO: 410, and the VL1 comprises the amino acid sequence of SEQ ID NO: 411;
i) the VH1 comprises the amino acid sequence of SEQ ID NO: 412, and the VL1 comprises the amino acid sequence of SEQ ID NO: 413;
j) the VH1 comprises the amino acid sequence of SEQ ID NO: 410, and the VL1 comprises the amino acid sequence of SEQ ID NO: 413;
k) the VH1 comprises the amino acid sequence of SEQ ID NO: 414, and the VL1 comprises the amino acid sequence of SEQ ID NO: 403;
1) the VH1 comprises the amino acid sequence of SEQ ID NO: 415, and the VL1 comprises the amino acid sequence of SEQ ID NO: 413;
m) the VH1 comprises the amino acid sequence of SEQ ID NO: 416, and the VL1 comprises the amino acid sequence of SEQ ID NO: 413; or
n) the VH1 comprises the amino acid sequence of SEQ ID NO: 416, and the VL1 comprises the amino acid sequence of SEQ ID NO: 411.

31. The multispecific antibody of claim 30, wherein the VH1 comprises the amino acid sequence of SEQ ID NO: 402, and the VL1 comprises the amino acid sequence of SEQ ID NO: 403.

32. The multispecific antibody of claim 30, wherein the VH1 comprises the amino acid sequence of SEQ ID NO: 410, and the VL1 comprises the amino acid sequence of SEQ ID NO: 411.

33-35. (canceled)

36. The multispecific antibody of claim 1, wherein the MM1 comprises the amino acid sequence of SEQ ID NO: 417.

37. The multispecific antibody of claim 1, wherein the CM1 comprises the amino acid sequence of SEQ ID NO: 77.

38. The multispecific antibody of claim 1, wherein the CM1 comprises the amino acid sequence of SEQ ID NO: 418.

39. (canceled)

40. (canceled)

41. The multispecific antibody of claim 1, wherein the target antigen is HER2.

42. The multispecific antibody of claim 41, wherein the second antigen-binding fragment comprises a VH2 and a VL2 of an anti-HER2 antibody, and wherein:
the VH2 comprises
a CDR-H1 comprising the amino acid sequence of SEQ ID NO: 423,
a CDR-H2 comprising the amino acid sequence of SEQ ID NO: 424, and
a CDR-H3 comprising the amino acid sequence of SEQ ID NO: 71; and
the VL2 comprises
a CDR-L1 comprising the amino acid sequence of SEQ ID NO: 72,
a CDR-L2 comprising the amino acid sequence of SEQ ID NO: 73, and
a CDR-L3 comprising the amino acid sequence of SEQ ID NO: 74.

43. The multispecific antibody of claim 42, wherein the VH2 comprises the amino acid sequence of SEQ ID NO: 75, and the VL2 comprises the amino acid sequence of SEQ ID NO: 76.

44. The multispecific antibody of claim 42, wherein:
a) the MM2 comprises an amino acid sequence according to Formula (XI): ESX1X₂CX₃X₄DPFX₅CQX₆ (SEQ ID NO: 670), wherein X₁ is D or E, X₂ is A, F, V, or Y, X₃ is D or E, X₄ is A or L, X₅ is D or E, and X₆ is A, F, or Y;
b) the MM2 comprises an amino acid sequence according to Formula (XII): X₁X₂X₃X₄X₅X₆CX₇X₈DPYECX₉X₁₀ (SEQ ID NO: 671), wherein X₁ is A, H, or S, X₂ is A, D, or S, X₃ is A, T, or V, X₄ is P, S, or T, X₅ is D or E, X₆ is A or V, X₇ is D or E, X₈ is A or L, X₉ is Q, S, or T, and X₁₀ is A, H, or V; or
c) the MM2 comprises an amino acid sequence according to Formula (XIII): YNSDDDCX₁SX₂YDPYTCYY (SEQ ID NO: 672), wherein X₁ is A, I, or V, and X₂ is H or R.

45. The multispecific antibody of claim 42, wherein the MM2 comprises an amino acid sequence selected from the group consisting of SEQ ID NOs: 36, 419, 432-476 and 491-515.

46. The multispecific antibody of claim 45, wherein the MM2 comprises the amino acid sequence of SEQ ID NO: 419.

47. The multispecific antibody of claim 46, wherein the CM2 comprises an amino acid sequence selected from the group consisting of SEQ ID NOs: 77, 127-129, 418, 420, 431, 477-490, and 516-555.

48. The multispecific antibody of claim 47, wherein the CM2 comprises the amino acid sequence of SEQ ID NO: 77 or 420.

49. The multispecific antibody of claim 48, comprising:
a) a first polypeptide comprising an amino acid sequence having at least 90% sequence identity with SEQ ID NO: 425,
a second polypeptide comprising an amino acid sequence having at least 90% sequence identity with SEQ ID NO: 426, and
a third polypeptide comprising an amino acid sequence having at least 90% sequence identity with SEQ ID NO: 112;
b) a first polypeptide comprising an amino acid sequence having at least 90% sequence identity with SEQ ID NO: 427,
a second polypeptide comprising an amino acid sequence having at least 90% sequence identity with SEQ ID NO: 428, and
a third polypeptide comprising an amino acid sequence having at least 90% sequence identity with SEQ ID NO: 112;
c) a first polypeptide comprising an amino acid sequence having at least 90% sequence identity with SEQ ID NO: 429,
a second polypeptide comprising an amino acid sequence having at least 90% sequence identity with SEQ ID NO: 430, and
a third polypeptide comprising an amino acid sequence having at least 90% sequence identity with SEQ ID NO: 115;
d) a first polypeptide comprising an amino acid sequence having at least 90% sequence identity with SEQ ID NO: 83,
a second polypeptide comprising an amino acid sequence having at least 90% sequence identity with SEQ ID NO: 84, and
a third polypeptide comprising an amino acid sequence having at least 90% sequence identity with SEQ ID NO: 85;
e) a first polypeptide comprising an amino acid sequence having at least 90% sequence identity with SEQ ID NO: 683,
a second polypeptide comprising an amino acid sequence having at least 90% sequence identity with SEQ ID NO: 684, and
a third polypeptide comprising an amino acid sequence having at least 90% sequence identity with SEQ ID NO: 685;
f) a first polypeptide comprising the amino acid sequence of SEQ ID NO: 425,
a second polypeptide comprising the amino acid sequence of SEQ ID NO: 426, and
a third polypeptide comprising the amino acid sequence of SEQ ID NO: 112;
g) a first polypeptide comprising the amino acid sequence of SEQ ID NO: 427,
a second polypeptide comprising the amino acid sequence of SEQ ID NO: 428, and
a third polypeptide comprising the amino acid sequence of SEQ ID NO: 112;
h) a first polypeptide comprising the amino acid sequence of SEQ ID NO: 429,
a second polypeptide comprising the amino acid sequence of SEQ ID NO: 430, and
a third polypeptide comprising the amino acid sequence of SEQ ID NO: 115;
i) a first polypeptide comprising the amino acid sequence of SEQ ID NO: 83,
a second polypeptide comprising the amino acid sequence of SEQ ID NO: 84, and
a third polypeptide comprising the amino acid sequence of SEQ ID NO: 85;
j) a first polypeptide comprising the amino acid sequence of SEQ ID NO: 683,
a second polypeptide comprising the amino acid sequence of SEQ ID NO: 684, and
a third polypeptide comprising the amino acid sequence of SEQ ID NO: 685;
k) a first polypeptide comprising the amino acid sequence of SEQ ID NO: 425 without the C-terminal lysine,
a second polypeptide comprising the amino acid sequence of SEQ ID NO: 426 without the C-terminal lysine, and
a third polypeptide comprising the amino acid sequence of SEQ ID NO: 112;
l) a first polypeptide comprising the amino acid sequence of SEQ ID NO: 427 without the C-terminal lysine,
a second polypeptide comprising the amino acid sequence of SEQ ID NO: 428 without the C-terminal lysine, and
a third polypeptide comprising the amino acid sequence of SEQ ID NO: 112;
m) a first polypeptide comprising the amino acid sequence of SEQ ID NO: 429 without the C-terminal lysine,
a second polypeptide comprising the amino acid sequence of SEQ ID NO: 430 without the C-terminal lysine, and
a third polypeptide comprising the amino acid sequence of SEQ ID NO: 115;
n) a first polypeptide comprising the amino acid sequence of SEQ ID NO: 83,
a second polypeptide comprising the amino acid sequence of SEQ ID NO: 84 without the C-terminal lysine, and
a third polypeptide comprising the amino acid sequence of SEQ ID NO: 85 without the C-terminal lysine; or
o) a first polypeptide comprising the amino acid sequence of SEQ ID NO: 683,
a second polypeptide comprising the amino acid sequence of SEQ ID NO: 684 without the C-terminal lysine, and
a third polypeptide comprising the amino acid sequence of SEQ ID NO: 685 without the C-terminal lysine.

50. The multispecific antibody of claim 1, wherein the target antigen is CD20.

51. The multispecific antibody of claim 50, wherein the second antigen-binding fragment comprises a VH2 and a VL2 of an anti-CD20 antibody, and wherein:
a) the VH2 comprises
a CDR-H1 comprising the amino acid sequence of SEQ ID NO: 556,
a CDR-H2 comprising the amino acid sequence of SEQ ID NO: 557, and
a CDR-H3 comprising the amino acid sequence of SEQ ID NO: 558; and
the VL2 comprises
a CDR-L1 comprising the amino acid sequence of SEQ ID NO: 559,
a CDR-L2 comprising the amino acid sequence of SEQ ID NO: 560, and
a CDR-L3 comprising the amino acid sequence of SEQ ID NO: 561; or
b) the VH2 comprises
a CDR-H1 comprising the amino acid sequence of SEQ ID NO: 86,
a CDR-H2 comprising the amino acid sequence of SEQ ID NO: 557, and
a CDR-H3 comprising the amino acid sequence of SEQ ID NO: 558; and
the VL2 comprises
a CDR-L1 comprising the amino acid sequence of SEQ ID NO: 559,
a CDR-L2 comprising the amino acid sequence of SEQ ID NO: 560, and
a CDR-L3 comprising the amino acid sequence of SEQ ID NO: 561.

52. The multispecific antibody of claim 51, wherein the VH2 comprises the amino acid sequence of SEQ ID NO: 562, and the VL2 comprises the amino acid sequence of SEQ ID NO: 563.

53. The multispecific antibody of claim 51, comprising:
a) a first polypeptide comprising an amino acid sequence having at least 90% sequence identity with SEQ ID NO: 564,
a second polypeptide comprising an amino acid sequence having at least 90% sequence identity with SEQ ID NO: 565, and
a third polypeptide comprising an amino acid sequence having at least 90% sequence identity with SEQ ID NO: 567;
b) a first polypeptide comprising an amino acid sequence having at least 90% sequence identity with SEQ ID NO: 564,
a second polypeptide comprising an amino acid sequence having at least 90% sequence identity with SEQ ID NO: 565, and
a third polypeptide comprising an amino acid sequence having at least 90% sequence identity with SEQ ID NO: 569;
c) a first polypeptide comprising the amino acid sequence of SEQ ID NO: 564,
a second polypeptide comprising the amino acid sequence of SEQ ID NO: 565, and
a third polypeptide comprising the amino acid sequence of SEQ ID NO: 567;
d) a first polypeptide comprising the amino acid sequence of SEQ ID NO: 564,
a second polypeptide comprising the amino acid sequence of SEQ ID NO: 565, and
a third polypeptide comprising the amino acid sequence of SEQ ID NO: 569;
e) a first polypeptide comprising the amino acid sequence of SEQ ID NO: 564,
a second polypeptide comprising the amino acid sequence of SEQ ID NO: 565 without the C-terminal lysine, and
a third polypeptide comprising the amino acid sequence of SEQ ID NO: 567 without the C-terminal lysine;
f) a first polypeptide comprising the amino acid sequence of SEQ ID NO: 564,
a second polypeptide comprising the amino acid sequence of SEQ ID NO: 565 without the C-terminal lysine, and
a third polypeptide comprising the amino acid sequence of SEQ ID NO: 569 without the C-terminal lysine.

54-134. (canceled)

135. The multispecific antibody of claim 8, wherein the VH1 comprises
a CDR-H1 comprising the amino acid sequence of SEQ ID NO: 390,
a CDR-H2 comprising the amino acid sequence of SEQ ID NO: 394, and
a CDR-H3 comprising the amino acid sequence of SEQ ID NO: 395; and
the VL1 comprises
a CDR-L1 comprising the amino acid sequence of SEQ ID NO: 397,
a CDR-L2 comprising the amino acid sequence of SEQ ID NO: 380, and
a CDR-L3 comprising the amino acid sequence of SEQ ID NO: 381.

136. The multispecific antibody of claim 135, wherein the ScFv comprises the amino acid sequence of SEQ ID NO: 422.

137. The multispecific antibody of claim 135, wherein the MM1 comprises the amino acid sequence of SEQ ID NO: 417.

138. The multispecific antibody of claim 136, wherein the MM1 comprises the amino acid sequence of SEQ ID NO: 417.

139. The multispecific antibody of claim 137, wherein the CM1 comprises the amino acid sequence of SEQ ID NO: 77.

140. The multispecific antibody of claim 138, wherein the CM1 comprises the amino acid sequence of SEQ ID NO: 77.

141. The multispecific antibody of claim 137, wherein the CM1 comprises the amino acid sequence of SEQ ID NO: 418.

142. The multispecific antibody of claim 138, wherein the CM1 comprises the amino acid sequence of SEQ ID NO: 418.

143. The multispecific antibody of claim 9, wherein the second polypeptide has the amino acid sequence of SEQ ID NO: 567.

144. The multispecific antibody of claim 143, wherein the first polypeptide has the amino acid sequence of SEQ ID NO: 565.

145. The multispecific antibody of claim 144, wherein the third polypeptide has the amino acid sequence of SEQ ID NO: 564.

146. The multispecific antibody of claim 18, wherein the second polypeptide has the amino acid sequence of SEQ ID NO: 85.

147. The multispecific antibody of claim 146, wherein the first polypeptide has the amino acid sequence of SEQ ID NO: 84.

148. The multispecific antibody of claim 147, wherein the third polypeptide has the amino acid sequence of SEQ ID NO: 83.

149. The multispecific antibody of claim 49, comprising:
a first polypeptide having an amino acid sequence of SEQ ID NO: 427,
a second polypeptide having an amino acid sequence of SEQ ID NO: 428, and
a third polypeptide having an amino acid sequence of SEQ ID NO: 112.

150. The multispecific antibody of claim 53, comprising:
a first polypeptide having an amino acid sequence having an amino acid sequence of SEQ ID NO: 564,
a second polypeptide having an amino acid sequence of SEQ ID NO: 565, and
a third polypeptide having an amino acid sequence of SEQ ID NO: 567.
